(19) United States
(12) Patent Application Publication
(10) Pub. No.: US 2021/0147525 A1
(43) Pub. Date: May 20, 2021

(54) METHODS AND COMPOSITIONS FOR TREATING PATHOGENIC BLOOD VESSEL DISORDERS

(71) Applicants: THE REGENTS OF THE UNIVERSITY OF CALIFORNIA, Oakland, CA (US); ATENGEN, INC., Los Angeles, CA (US)

(72) Inventors: HUI SUN, Culver City, TX (US); Adrian Chichuen Au, Los Angeles, CA (US); Guo Cheng, Los Angeles, CA (US); Pu Sun, San Jose, CA (US)

(21) Appl. No.: 17/072,952

(22) Filed: Oct. 16, 2020

Related U.S. Application Data
(60) Provisional application No. 62/916,953, filed on Oct. 18, 2019.

(51) Int. Cl. C07K 16/18

(52) U.S. Cl. CPC C07K 16/18; C07K 2317/565

(57) ABSTRACT

Pathogenic blood vessels are blood vessels that are not involved in the vascularization of normal organs but are in the pathogenic tissues such as the new blood vessels that drive vision diseases or the blood vessels in tumors that tumors depend on to survive. The current disclosure provides an advancement over typical anti-angiogenic strategies that target the generation of new blood vessels by providing compositions and methods that can selectively target and kill existing pathogenic blood vessels. The conventional antiangiogenic drugs or factors inhibit angiogenesis (the growth of new blood vessels), but cannot effectively kill existing pathogenic blood vessels. Agents, including small molecule compounds and antibodies, have been identified that bind to and activate PLXDC1 and PLXDC2 proteins, leading to effective killing of the endothelial cells in pathogenic blood vessels in vision diseases and in tumors that express these proteins. The disclosure thereby provides a novel modality for eliminating or reducing existing pathogenic blood vessels, thereby treating diseases such as diabetic retinopathy, age-related macular degeneration (AMD), retinopathy of prematurity, and cancer.

### CROSS REFERENCE TO RELATED APPLICATIONS

This application claims the benefit under 35 U.S.C. § 119(e) of U.S. Provisional Application Ser. No. 62/916,953 filed on Oct. 18, 2019, the content of which is incorporated by reference in its entirety into the present disclosure.

### BACKGROUND

### I. Field

This invention relates to the field of medicine and therapeutic compositions and methods for treating cancer and other pathogenic blood vessel disorders.

### II. Background

Angiogenesis plays a key role in the pathogenesis of several major human diseases (Carmeliet, 2005). In addition to tumor growth and metastasis, angiogenesis is a major driving force in several blinding diseases including diabetic retinopathy, age-related macular degeneration (AMD), and retinopathy of prematurity. AMD and diabetic retinopathy are the leading causes of blindness in the elderly and populations at the working age in the United States, respectively. Retinopathy of prematurity is a common reason that causes the loss of vision for newborn babies.

Angiogenesis also plays a role in pathogenesis of cancer, e.g., tumor development, since newly-formed blood vessels supply the tumor with growth nutrients and signals that allow the tumor to grow and spread. Accordingly, cutting off a tumor's supply of nutrients and primary mechanism for traveling to distant sites is an attractive therapeutic strategy. However, current anti-angiogenic strategies only target newly formed blood vessels, and are unable to target existing blood vessels that contribute to disease progression.

Different disease progression patterns can be induced by anti-angiogenic therapies, which may lead to worse outcomes in terms of drug resistance, invasion, and metastasis. Furthermore, targeting angiogenesis does not treat existing blood vessels that may have, for example, already vascularized a tumor. There is a need in the art for complementary therapies that, in contrast to anti-angiogenic therapies, can target existing blood vessels and treat cancer and other disorders arising from angiogenesis (collectively referred to herein as pathogenic blood vessel disorders).

### SUMMARY

The current disclosure provides an advancement over the conventional anti-angiogenic strategies that target the generation of new blood vessels, by providing compositions and methods that also can selectively target and kill existing pathogenic blood vessels. As illustrated in FIG. 8A-C, tumor development and survival relies on vascularization for the supply of growth factors and nutrients and as a mechanism for metastasizing to distant sites. The conventional antiangiogenic drugs or factors inhibit angiogenesis, the growth of the tumor, but cannot kill the tumor because they cannot effectively kill existing tumor blood vessels. As such, described herein are methods for treating a disorder in a patient, wherein the disorder is characterized with pathogenic blood vessels and the method comprises activating a plexin domain-containing (PLXDC) protein (e.g., PLXDC1 and PLXDC2) expressed in the pathogenic blood vessels in the patient. Described herein are novel compounds and antibodies that activate the PLXDC1 or the PLXDC2 proteins and lead to the effective killing of the endothelial cells, thereby providing a novel modality for eliminating or reducing a primary mechanism of pathogenicity.

It is worth noting that, prior to the instant disclosure, no small molecule that targets PLXDC1/PLXDC2 to kill endothelial cells previously exists. Also, no antibody that targets PLXDC1/PLXDC2 to kill endothelial cells previously exists. The small molecules and antibodies disclosed herein target PLXDC1/PLXDC2 to kill endothelial cells. Because PLXDC1/PLXDC2 is highly enriched in pathogenic blood vessels such as tumor blood vessels and have no or non-detectable expression in most healthy blood vessels, these agents can kill pathogenic blood vessels with specificity.

Identification of such agents, in particular the antibodies, was a surprise. To the best knowledge of the instant inventors, all existing antibody drugs are neutralizing antibodies or targeting antibodies. Neutralizing antibodies inhibit the ligand/receptor interaction, such as Humira (inhibiting TNF-α, a ligand), Avastin (inhibiting VEGF, a ligand), Herceptin (inhibiting HER2, a receptor), and Keytruda (inhibiting PD-1, a receptor). Targeting antibodies may exert their functions through mechanisms such as antibody-drug conjugates and antibody-dependent cell-mediated cytotoxicity (ADCC). No activating antibodies have been identified, in particular against single transmembrane cell-surface receptors like PLXDC1/PLXDC2.

Aspects of the disclosure relate to a method for treating a pathogenic blood vessel-related disorder in a patient. Further aspects relate to a method for treating a disorder in a patient, wherein the disorder is characterized with pathogenic blood vessels and the method comprises activating a plexin domain-containing (PLXDC) protein expressed in the pathogenic blood vessels in the patient. Activating the PLXDC protein may comprise administration of an agent that binds to the PLXDC protein.

In some embodiments, the method comprises activating a plexin domain-containing (PLXDC) protein (e.g., PLXDC1 or PLXDC2) in the pathogenic blood vessel. In some embodiments, the method comprises administering to the patient a plexin domain-containing (PLXDC) protein binding agent. The PLXDC protein, in some embodiments, is a PLXDC1 protein or a PLXDC2 protein. The binding agent can be a small molecule or a polypeptide such as an antibody.

Further aspects relate to a method comprising: a) obtaining a sample comprising cells, and b) determining whether the cells express PLXDC1 (Plexin Domain-Containing 1) or PLXDC2 (Plexin Domain-Containing 2).

Further aspects of the disclosure relate to anti-PLXDC1 or anti-PLXDC2 antibodies and antigen binding fragments, which can be used for treating the disorders. In some embodiments, the antibody is selected from Table 6, is an antibody that includes the CDRs of any antibody of Table 6, or is an antibody that binds to the same epitope as any antibody of Table 6. In some embodiments, the antibody is selected from AA02, AA03, or AA94; an antigen binding fragment of AA02, AA03, or AA94; or a humanized or chimeric version of AA02, AA03, or AA94. Also provided are compositions, comprising an antibody or antigen binding fragment of the disclosure, nucleic acids encoding for antibodies or antigen binding fragments of the disclosure, and host cells comprising antibodies, antigen binding fragments, or nucleic acids of the disclosure.

In further aspects, the disclosure provides compounds, and compositions, including pharmaceutical compositions, kits that include the compounds, and methods of using (or administering) and making the compounds. The disclosure further provides compounds or compositions thereof for use in a method of modulating PLXDC1 (TEM7) and/or PLXDC2 or killing pathogenic blood vessels. The disclosure further provides compounds or compositions thereof for use in a method of treating a disease, disorder, or condition that is mediated, at least in part, by PLXDC1/PLXDC2 or by angiogenesis.

The disclosure also relates to an antibody or antigen binding fragment thereof having specificity to the human plexin domain-containing 1 (PLXDC1) protein, wherein the antibody or antigen binding fragment thereof comprises a heavy chain variable region (VH) and a light chain variable region (VL), the VH comprises a VH complementarity-determining region (CDR) CDR1, a VH CDR2, a VH CDR3, the VL comprises a VL CDR1, a VL CDR2, and a VL CDR3, wherein the VH CDR1, VH CDR2, VH CDR3, VL CDR1, VL CDR2, and VL CDR3 comprises the VH CDR1, VH CDR2, VH CDR3, VL CDR1, VL CDR2, and VL CDR3, respectively, of an antibody selected from Table 6. Further aspects relate to an antibody or antigen binding fragment thereof having specificity to the human plexin domain-containing 1 (PLXDC1) protein, wherein the antibody or antigen binding fragment thereof competes with an antibody selected from Table 6 in binding to PLXDC1. Also provided is an antibody or antigen binding fragment thereof having specificity to the human plexin domain-containing 1 (PLXDC1) protein, wherein the antibody or antigen binding fragment thereof competes with an antibody selected from Table 6 in binding to PLXDC1.

Further aspects of the disclosure relate to compositions comprising the antibodies, nucleic acid(s) encoding the antibodies, and host cells comprising the antibodies or nucleic acids.

The disclosure also provides a method for identifying an activator of a PLXDC protein, comprising contacting a candidate molecule with the PLXDC protein in the presence of a reference PLXDC activator, and detecting the binding affinity between the candidate molecule and the PLXDC protein, thereby identifying the candidate molecule as a PLXDC activator when the detected binding affinity is greater than a reference binding affinity between the candidate molecule and the PLXDC protein in the absence of the reference PLXDC activator. Also described is a method for treating a disorder in a patient, wherein the disorder is characterized with pathogenic blood vessels and the method comprises administering to the patient the an antibody of the disclosure.

In some embodiments, the binding agent binds to PLXDC1. In some embodiments, the binding agent binds to PLXDC2. In some embodiments, the binding agent comprises a small molecule. In some embodiments, the binding agent comprises a polypeptide. In some embodiments, the polypeptide or binding agent comprises a PLXDC1/PLXDC2 antibody or an antigen binding fragment thereof. In some embodiments, the binding agent comprises a fusion polypeptide. In some embodiments, the antibody comprises AA02, AA03, or AA94. In some embodiments, the binding agent comprises an antigen binding fragment of AA02, AA03, or AA94. In some embodiments, the binding agent comprises a humanized or chimeric version of AA02, AA03, or AA94. In some embodiments, the antigen binding fragment or antibody comprises one or both of a heavy chain variable region and a light chain variable region from a PLXDC1 antibody. In some embodiments, the heavy chain variable region comprises HCDR1, HCDR2, and HCDR3 from the heavy chain variable region of a PLXDC1 antibody. In some embodiments, the light chain variable region comprises LCDR1, LCDR2, and LCDR3 from the light chain variable region of a PLXDC1 antibody.

In some embodiments, the antibody or antigen binding agent thereof is not capable of mediating antibody-dependent cell-mediated cytotoxicity (ADCC). In some embodiments, the antibody or antigen binding fragment thereof binds to the PLXDC protein with a higher affinity in the presence of a small molecule compound that binds and activates the PLXDC protein, as compared to when the small molecule compound is not present.

In some embodiments, the binding agent binds to domain A of PLXDC1. In some embodiments, the binding agent binds to domain B of PLXDC1. In some embodiments, the agent binds to an amino acid residue of PLXDC that is not exposed in the basal state. In some embodiments, the binding agent binds to domain C of PLXDC1. In some embodiments, the binding agent binds to domain D of PLXDC1. In some embodiments, the binding agent binds to domain E of PLXDC1. In some embodiments, the binding agent does not bind to domain B of PLXDC1. In some embodiments, the binding agent binds to domain A of PLXDC2. In some embodiments, the binding agent binds to domain B of PLXDC2. In some embodiments, the binding agent binds to domain C of PLXDC2. In some embodiments, the binding agent binds to domain D of PLXDC2. In some embodiments, the binding agent binds to domain E of PLXDC2. In some embodiments, the binding agent does not bind to domain B of PLXDC1.

In some embodiments, the antibody or antigen binding fragment thereof is an antibody selected from Table 6 or an antigen binding fragment thereof, is an antibody or antigen binding fragment thereof that includes the complementarity-determining regions (CDR) VH CDR1, VH CDR2, VH CDR3, VL CDR1, VL CDR2 and VL CDR3 of the antibodies selected from Table 6, or is an antibody or antigen binding fragment thereof that competes with an antibody selected from Table 6 in binding to PLXDC1.

In some embodiments, the binding agent activates the PLXDC. In some embodiments, the binding agent induces NFκB activation. In some embodiments, treating comprises inducing NFκB activation in pathogenic blood vessels. In some embodiments, treating comprises increasing necrosis of pathogenic blood vessels. In some embodiments, the pathogenic blood vessel-related disorder comprises diabetic retinopathy, age-related macular degeneration (AMD), retinopathy of prematurity, or cancer. In some embodiments, the pathogenic blood vessel-related disorder comprises cancer. In some embodiments, the cancer comprises colon cancer. In some embodiments, the cancer comprises lung cancer. In some embodiments, the cancer comprises a solid tumor. In some embodiments, the cancer comprises a vascularized tumor.

In some embodiments, the binding agent comprises a small molecule. In some embodiments, the small molecule is a compound of Formula I. In some embodiments, the small molecule is a compound identified by compound No. in Table 4 or 5. In some embodiments, the small molecule is a compound that has an activity of inducing at least 60% of endothelial cells to vesicularize in cell shape. In some embodiments, the small molecule is a compound that has an activity of inducing at least 80% of endothelial cells to vesicularize in cell shape. In some embodiments, the small molecule is a compound that has an activity of inducing at least 95% of endothelial cells to vesicularize in cell shape. In some embodiments, the small molecule is a compound that has an activity of inducing at least 60, 65, 70, 75, 80, 85, 90, 95, or 100% of endothelial cells (or any derivable range therein) to vesicularize in cell shape. In some embodiments, the small molecule is a compound that has an activity of inducing at least 50% of cell death. In some embodiments, the small molecule is a compound that has an activity of inducing at least 80% of cell death. In some embodiments, the small molecule is a compound that has an activity of inducing 95% of cell death. In some embodiments, the small molecule is a compound that has an activity of inducing at least 60, 65, 70, 75, 80, 85, 90, 95, or 100% (or any derivable range therein) of cell death.

In some embodiments, the agent is an antibody or antigen binding fragment thereof that has an activity of inducing at least 95% of endothelial cells to vesicularize in cell shape. In some embodiments, the agent is an antibody or antigen binding fragment thereof that has an activity of inducing at least 60, 65, 70, 75, 80, 85, 90, 95, or 100% of endothelial cells (or any derivable range therein) to vesicularize in cell shape. In some embodiments, the agent is an antibody or antigen binding fragment thereof that has an activity of inducing at least 50% of cell death. In some embodiments, the agent is an antibody or antigen binding fragment thereof that has an activity of inducing at least 80% of cell death. In some embodiments, the agent is an antibody or antigen binding fragment thereof that has an activity of inducing 95% of cell death. In some embodiments, the agent is an antibody or antigen binding fragment thereof that has an activity of inducing at least 60, 65, 70, 75, 80, 85, 90, 95, or 100% (or any derivable range therein) of cell death.

In some embodiments of the methods and compositions provided herein comprise combination of agents such as at least 1, 2, 3, or 4 antibodies, compounds, or mixtures thereof. Exemplary therapeutic compositions and regimens include administration of, or a composition comprising, at least one antibody or antigen binding fragment thereof and at least one compound of the disclosure.

In some embodiments, the binding agent does not compete with PEDF (Pigment epithelium-derived factor) for binding to the PLXDC1 or PLXDC2 protein. In some embodiments, the agent is not PEDF or a mimetic thereof. In some embodiments, the disorder is selected from the group consisting of diabetic retinopathy, age-related macular degeneration (AMD), retinopathy of prematurity, cancer and combinations thereof. In some embodiments, the pathogenic blood vessel-related disorder comprises cancer and further wherein the patient is one that has a malignant tumor. In some embodiments, the tumor comprises a solid tumor. In some embodiments, the tumor has a diameter of greater than 2 cm. In some embodiments, the tumor has a diameter of at least, or at most 1, 2, 3, 4, 5, 6, 7, or 8 cm (or any range derivable therein).

In some embodiments, the binding agent specifically induces endothelial cell necrosis in the targeted blood vessels. In some embodiments, the binding agent does not directly induce tumor cell necrosis. In some embodiments, treating comprises inducing and/or increasing coagulative necrosis in a tumor in the patient. In some embodiments, treating comprises inducing and/or increasing infarction in the tumor. In some embodiments, the patient has been determined to have pathogenic blood vessels. In some embodiments, the patient has been determined to have PLXDC1 and/or PLXDC1-expressing cells. In some embodiments, the expressing cells comprise endothelial cells. In some embodiments, the expressing cells comprise cell surface expression of PLXDC1 and/or PLXDC2.

In some embodiments, the patient has previously been treated for the pathogenic blood vessel-related disorder with an additional therapy. In some embodiments, the patient has been determined to be non-responsive or have a toxic response to the additional therapy. In some embodiments, the additional therapy comprises an anti-angiogenic therapy. In some embodiments, the additional therapy comprises an immunotherapy. In some embodiments, the patient has not previously been treated for the pathogenic blood vessel-related disorder.

In some embodiments, the candidate molecule is an antibody or antigen binding fragment. The reference PLXDC activator may be a small molecule compound in method embodiments of the disclosure.

In some embodiments of the disclosure, the cell(s) comprise endothelial cells. In some embodiments, b) comprises determining whether the endothelial cells express PLXDC1 (Plexin Domain-Containing 1) or PLXDC2 (Plexin Domain-Containing 2) on the cell surface of the endothelial cell. In some embodiments, b) comprises contacting the endothelial cells with a PLXDC1 or PLXDC2 binding agent and detecting the binding of the endothelial cells to the binding agent or the absence of the binding of the endothelial cells to the binding agent. In some embodiments, b) comprises ELISA, FACS, or a western blot. In some embodiments, the method further comprises determining the level of NFkB activation in the cells compared to a control. In some embodiments, the control comprises endothelial cells not contacted with a binding agent. In some embodiments, the method of the disclosure comprises an in vitro method.

In some embodiments, the antigen-binding fragment comprises a scFv, a diabody, or a single domain antibody. In some embodiments, the antibody or antigen binding fragment is conjugated to one or more adaptor polypeptides. In some embodiments, the one or more adaptor polypeptides comprise a serum protein. In some embodiments, the serum protein comprises albumin. In some embodiments, the one or more adaptor polypeptides comprises a therapeutic polypeptide. In some embodiments, the antibody or antigen binding fragment is conjugated to a therapeutic agent. In some embodiments, the therapeutic agent comprises a cytotoxic agent.

Further aspects relate to a method comprising expressing the one or more nucleic acids of the disclosure in a cell and isolating polypeptides expressed from the nucleic acid(s). Further method aspects relate to a method comprising contacting an antibody of the disclosure with a PLXDC1 or PLXDC2 polypeptide. In some embodiments, the PLXDC1 or PLXDC2 polypeptide comprises Domain A. In some embodiments, the PLXDC1 or PLXDC2 polypeptide comprises Domain B. In some embodiments, the PLXDC polypeptide comprises Domain C. In some embodiments, the PLXDC1 or PLXDC2 polypeptide comprises Domain D In some embodiments, the PLXDC1 or PLXDC2 polypeptide comprises Domain E. Further aspects of the disclosure relate to a method comprising testing a composition of the disclosure for one or more contaminants.

In certain embodiments, provided are compounds of Formula (I) or a pharmaceutically acceptable salt or prodrug thereof, wherein Formula (I) is

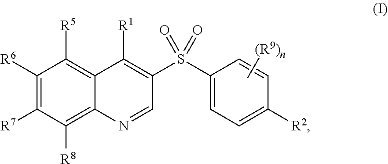

wherein each of n, R¹, R², R⁵, R⁶, R⁷, R⁸ and R⁹ is as defined herein.

In certain embodiments, provided is a pharmaceutical composition comprising a compound as described herein, or a pharmaceutically acceptable salt or prodrug thereof, and a pharmaceutically acceptable carrier.

In certain embodiments, provided is a method for treating a disease or disorder that is mediated, at least in part, by PLXDC1 or PLXDC2 in a subject in need thereof, the method comprising administering to the subject an effective amount of a compound or a pharmaceutical composition comprising a compound as described herein, or a pharmaceutically acceptable salt or prodrug thereof.

The disclosure also provides uses of the compounds, or a pharmaceutically acceptable salt or prodrug thereof, in the manufacture of a medicament for modulating PLXDC. Moreover, the disclosure provides uses of the compounds, or a pharmaceutically acceptable salt or prodrug thereof, in the manufacture of a medicament for the treatment of a disease, disorder, or condition that is mediated, at least in part, by PLXDC1 or PLXDC2.

The disclosure also provides use of the compounds, or a pharmaceutically acceptable salt or prodrug thereof, in treating a disease, such as cancer, retinal occlusive vascular disease, retinopathy of prematurity, diabetic retinopathy, and age-related macular degeneration.

Other embodiments and assays useful in the methods and compositions of the disclosure are described in U.S. Patent Application Nos.: 62/923,029, 62/916,997, 62/916,983, and PCT/US20/55979, each of which are incorporated by reference. Furthermore, the assays described in U.S. Patent Application Nos.: 62/923,029 and 62/916,997 may be used for determining the activity of the agents described herein.

It is specifically contemplated that any limitation discussed with respect to one embodiment of the invention may apply to any other embodiment of the disclosure. Furthermore, any composition of the disclosure may be used in any method of the disclosure, and any method of the disclosure may be used to produce or to utilize any composition of the disclosure. Aspects of an embodiment set forth in the Examples are also embodiments that may be implemented in the context of embodiments discussed elsewhere in a different Example or elsewhere in the application, such as in the Summary, Detailed Description, Claims, and description of Figure Legends.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1A-H. Expression of PLXDC1 in pathogenic blood vessels in choroidal neovascularization (CNV) and ischemia-induced retinopathy. Red channel shows blood vessel marker Griffonia Simplicifolia Lectin I-isolectin B4. Green channel shows anti-PLXDC1 signal. A-D. Highly enriched PLXDC1 expression in pathogenic blood vessels in a mouse model of CNV (laser-induced CNV). A&B, retina sections. Arrowheads indicate examples of normal inner retinal blood vessels (in A) that are negative for PLXDC1 signal (in B). C&D, staining done on flat-mounted eye cup. E-H. High expression of PLXDC1 in pathogenic blood vessels in a mouse model of ischemia-induced retinopathy, but not in blood vessels of healthy retina. E&F. P17 retina of ischemia-induced retinopathy (E and F are the same section stained by endothelial cell marker and PLXDC1 antibody, respectively). Examples of pathogenic blood vessels expressing PLXDC1 are indicated by white arrows. G&H. P17 healthy retina (G and H are the same section stained by endothelial cell marker and PLXDC1 antibody, respectively). Examples of healthy blood vessels showing no detectable PLXDC1 expression are indicated by white arrows in G (there is no corresponding PLXDC1 signals in H). CH, choroid. ON, outer nuclear layer. IN, inner nuclear layer. GC, ganglion cell layer.

FIG. 2A-E. Comparison of compound 369 (Table 3) with the current anti-angiogenic drug in an ex vivo model of choroidal angiogenesis. A. A schematic diagram of the timeframe of the experiment. Treatment does not start until choroidal angiogenesis occurs for 7 days. Treatment lasts for two days before cell death and survival are analyzed. B. Control experiment without any drug treatment at day 7. The white circle in the middle delineates the piece of choroid/RPE that was embedded to initiate neovascularization. C. The most commonly used drug for choroidal neovascularization, Eylea, can inhibit choroidal endothelial cell growth (as expected of an antiangiogenesis drug). Eylea was added at 10 μM. D. Compound 369 that targets PLXDC1/PLXDC2 can kill the new endothelial cells in choroidal angiogenesis. Choroid and RPE are still alive after the treatment, demonstrating the high specificity of the treatment. The compound was added at 10 μM. In B-D, green cells are live cells and red cells are dead cells. E. Quantitation of the experiments described in B-D. The amount of new endothelial cells in the untreated control is defined as 100%.

FIG. 3A-B. Choroidal neovascularization in an AMD patient treated by a commonly used anti-VEGF drug (Eylea). After 24 injections between 2015 and 2018, the pathogenic blood vessels still grew slightly and are not killed by the treatment. Fundus pictures of the pathogenic blood vessels in 2015 (A) and 2018 (B) are shown.

FIG. 4A-F. A comparison between an anti-VEGF drug, PEDF and two compounds that target PLXDC1/PLXDC2 in an ex vivo model of tumor angiogenesis. PEDF can inhibit tumor endothelial cell growth (as expected of an antiangiogenesis factor), but two compounds that target PLXDC1/PLXDC2 can kill tumor endothelial cells. Anti-VEGF drug (VEGF trap) is not very effective in this experiment due to the presence of other angiogenic factors such as bFGF and EGF in the culture media. Tumor endothelial cells were grown for 5 days before test reagents were added for 2 days. Green cells are live cells and red cells are dead cells.

FIG. 5A-C. Killing of human tumor endothelial cells by antibodies against human PLXDC1. In 3D ex vivo model of human tumor endothelial cells, anti-human PLXDC1 monoclonal antibody clones AA03 and AA94 kill human tumor endothelial cells. The tumor is a from human colon cancer. Green cells represent live cells. Red cells represent dead cells.

FIG. 6A-B. Killing of human tumor endothelial cells by antibodies against human PLXDC1. In 3D ex vivo model of human tumor endothelial cells, anti-human PLXDC1 monoclonal antibody clones AA02, but a control antibody, kills human tumor endothelial cells. The tumor is from human liver cancer. Green cells represent live cells. Red cells represent dead cells.

FIG. 7A-D. Systemic administration of a compound that targets PLXDC1 and PLXDC2 kills existing tumor blood vessels and leads to massive tumor necrosis. A. A control mouse that received systemic vehicle treatment has a live tumor, which shows reddish appearance indicative of a rich blood supply. B. A mouse that received systemic treatment by compound 369 that targets PLXDC1/PLXDC2 has a dead tumor, which shows yellowish appearance indicative of a dead tissue without blood supply. The high specificity of the treatment is illustrated by the healthy skin blood vessels that cover the dead yellow tumor. C. The tumor from control mouse in A is cut open to reveal that it is rich in tumor blood vessels and is a live tumor. D. The tumor from compound-treated mouse in B is cut open to reveal a lack of tumor blood vessels and a necrotic tumor with yellow cheese-like appearance (coagulative necrosis). Coagulative necrosis is exactly what is expected for a treatment that can kill existing tumor blood vessels to block major blood supplies to the tumor. Precise and efficient killing of tumor blood vessels to cause massive tumor death by necrosis, which has been achieved by the compound 369 targeting PLXDC1/PLXDC2, cannot be achieved by any existing drugs or known antiangiogenic factors.

FIG. 8A-C. A comparison between antiangiogenesis treatment and killing existing tumor blood vessels by targeting PLXDC1/PLXDC2. Angiogenesis is the growth of new blood vessels from existing blood vessels. A. Tumor depends on angiogenesis to grow and to metastasize. B. Antiangiogenic drugs or factors inhibit angiogenesis and the growth of the tumor, but cannot kill the tumor because they cannot effectively kill existing tumor blood vessels. C. Drugs that target PLXDC1/PLXDC2 can kill existing tumor blood vessels to cause tumor death. This therapeutic effect goes beyond antiangiogenesis, which means the inhibition of new blood vessel growth.

FIG. 9A-C. Schematic diagrams of the experimental strategy to detect the binding of compound 369 to PLXDC1. A. Schematic diagram of the compound structure. The compound was designed to have an albumin binding structure and structures responsible for PLXDC1 interaction. B. Schematic diagram of the binding of the compound to human albumin, which has been biotinylated (Biotin-HSA-Compound). C. Schematic diagram of the strategy to detect the binding of Biotin-HSA-Compound to PLXDC1's extracellular domain After binding, the biotin in the complex is detected by avidin-alkaline phosphatase.

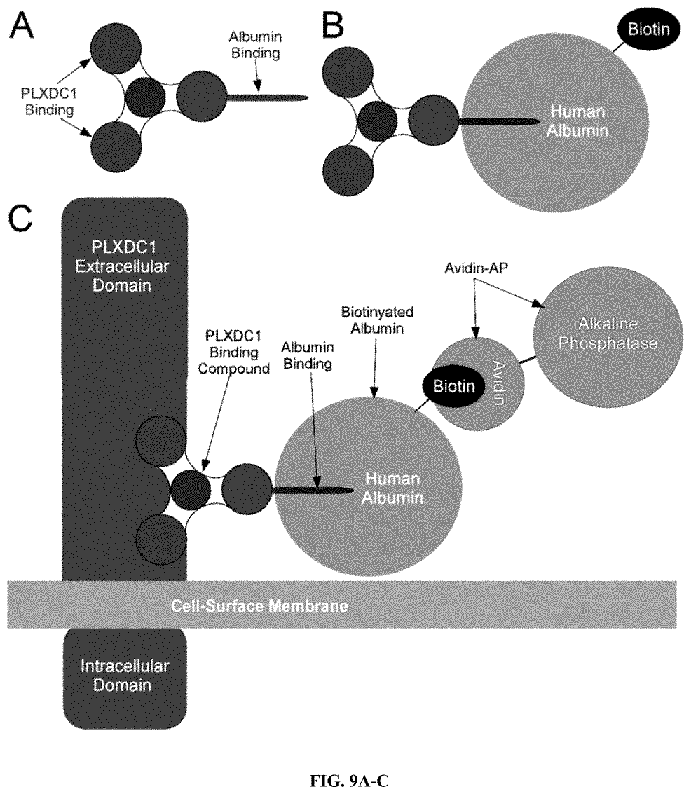

FIG. 10A-D. The compound that kills endothelial cells expressing PLXDC1 interacts with PLXDC1 independently of Domain B. Left Picture. Schematic diagrams of the domains in PLXDC1 (domains A to E on the extracellular side, the transmembrane domain and the intracellular domain) and the constructs used in this experiment. The domains for human PLXDC1 are defined as: Domain A (20 to 127), Domain B (128 to 242), Domain C (243 to 292), Domain D (293 to 359) and Domain E (360-427). Residues are numbered according to the full-length PLXDC1 without the secretion signal. All constructs in this experiment include an epitope tag (labeled as “R”) at the N-terminus. R-PLXDC1=full length human PLXDC1; R-AB-Del=domain A-B-deleted human PLXDC1. A-D. Binding of biotinylated-HSA (Biotin-HSA) or biotinylated-HAS-compound complex (Biotin-HSA-Compound) to HEK293 cells transfected with the constructs. Purple color represents the binding signal. A=full length human PLXDC1 transfected cells; B=control untransfected cells; C=full length human PLXDC1 transfected cells; D=Domain A-B deleted human PLXDC1 transfected cells. Biotin-HSA-Compound was added to B-D, while Biotin-HSA was added to A.

FIG. 11A-B. The monoclonal antibodies that kill endothelial cells expressing PLXDC1 interact with PLXDC1 independently of Domain B. A. Schematic diagrams of the domains in PLXDC1 (domains A to E on the extracellular side, the transmembrane domain and the intracellular domain) and the constructs used in this experiment. The domains for human PLXDC1 are defined as: Domain A (20 to 127), Domain B (128 to 242), Domain C (243 to 292), Domain D (293 to 359) and Domain E (360-427). Residues are numbered according to the full-length PLXDC1 without the secretion signal. All constructs in this experiment include an epitope tag (labeled as “R”) at the N-terminus. R-PLXDC1=full length human PLXDC1; R-AB-Del=domain A-B-deleted human PLXDC1; R-PLXDC2=full length human PLXDC2. B. Live cell staining of HEK293 cells transfected with the constructs using different antibodies. Red signal is immunostaining signal. Blue signal is a DNA stain that indicates cell nucleus. R antibody recognizes the R epitope on all constructs. Monoclonal antibodies AA02 and AA03 are capable of killing endothelial cells expressing PLXDC1. They recognizes all constructs except human PLXDC2, suggesting that they bind to PLXDC1 independently of Domain B.

FIG. 12 shows that monoclonal antibodies activated PLXDC1. An assay of receptor-induced NF-κB activation. Addition of anti-human PLXDC1 monoclonal antibodies Ab-AA02, Ab-AA03 and Ab-AA94 stimulate PLXDC1-induced NF-κB activation. Both Ab-AA02 and Ab-AA03 recognize domain E of PLXDC1. Basal NF-κB activation in PLXDC1 expressing cells is defined as 1.

FIG. 13 illustrates a few different strategy of killing tumors.

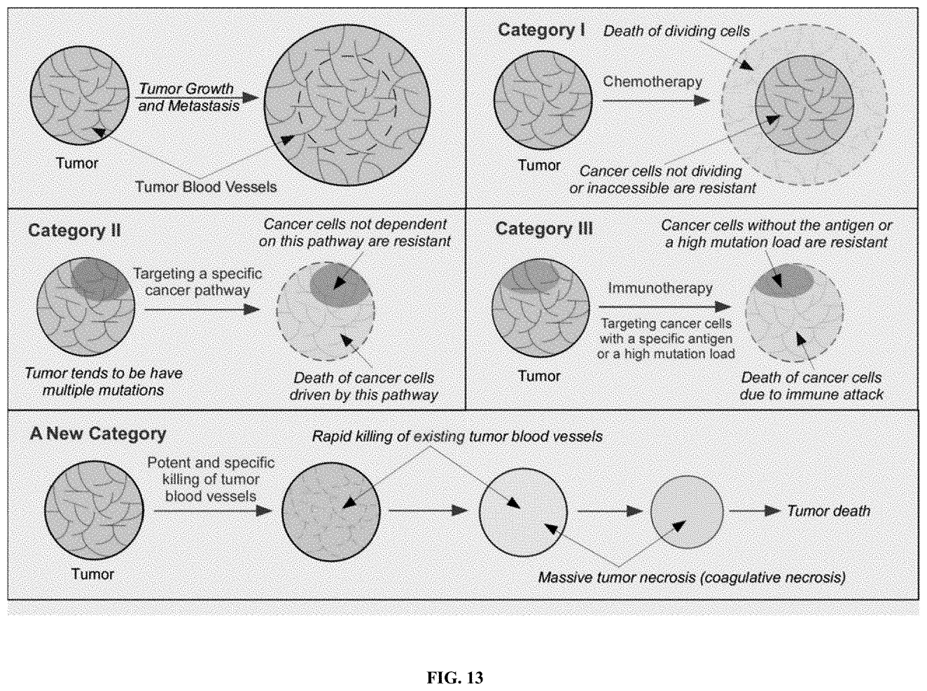

FIG. 14A-B show tumor shrinkage and necrosis following treatment with compounds described herein. FIG. 14A shows that 3 days after injection, all the tumors were shrinking FIG. 14B shows that the tumor shrinkage was maintained 6 days after injection.

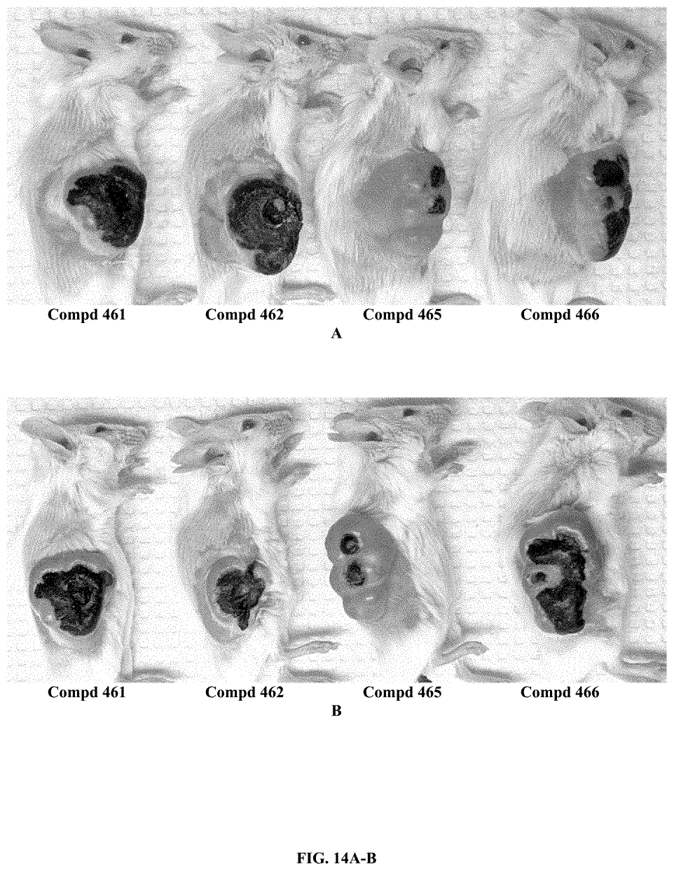

FIG. 15A-B show high affinity interaction between PLXDC1-activating compounds and the extracellular domain of PLXDC1 (PLXDC1-ECD). A. Raw data of the tryptophan fluorescence of PLXDC1-ECD as measured in a fluorometer after adding different concentrations of the compound. B. Dose-dependent curve of the suppression of tryptophan fluorescence. Tryptophan fluorescence without compound added is defined as 1. The estimated Kd value is 50 nM.

FIG. 16A-E show specific binding of PLXDC1-activating antibodies to human PLXDC1 expressed on live cells. A. Binding of an antibody against the epitope tag demonstrated the expression of human PLXDC1 (left picture) and human PLXDC2 (middle picture) in their transfected cells, while it does not bind to untransfected cells (right picture). The green signal is the antibody binding signal. B & C. Binding of receptor activating antibody (3-G7/A-TEM7-Ab-1) to cells transfected with human PLXDC1 (left picture). This antibody does not bind to human PLXDC2 (middle picture) or untransfected control cells (right picture). The green signal in B and C is the antibody binding signal. The blue signal in C represent cell nuclear staining by the DNA dye DAPI. D & E. Binding of receptor activating antibody (8-C9/A-TEM7-Ab-2) to cells transfected with human PLXDC1 (left picture). This antibody does not bind to human PLXDC2 (middle picture) or untransfected control cells (right picture). The green signal in D and E is the antibody binding signal. The blue signal in E represent cell nuclear staining by the DNA dye DAPI.

FIG. 17A-B show binding avidity of PLXDC1 receptor-activating antibodies to PLXDC1 extracellular domain (PLXDC1-ECD). A. PLXDC1 receptor-activating antibody 3-G7 (A-TEM7-Ab-1) bound to PLXDC1-ECD with high avidity (9.6 nM). In the presence of a small molecule (Compound) that can activate PLXDC1, the avidity is increased to 1.5 nM. B. PLXDC1 receptor-activating antibody 3-G7 (A-TEM7-Ab-1) binds to PLXDC1-ECD with high avidity (1.9 nM). In the presence of a small molecule (Compound) that can activate PLXDC1, the avidity is increased to 0.9 nM.

FIG. 18A-B show activation of PLXDC1 and PLXDC2 by small molecules. Through RNAseq analysis of PLXDC1-expressing endothelial cells killing by PLXDC1-activating compounds, a transcriptional factor called Gfi1b was found to be induced during PLXDC1-mediated cell killing By linking its promotor to a luciferase reporter gene, this example developed a PLXDC1 receptor activation assay that demonstrates the activation of the receptor by its ligands. A. PLXDC1-activating compounds (A-Com-1 and A-Com-2) highly activated the promotor activity in PLXDC1-expressing cells. B. A-Com-1 and A-Com-2 also activated the promotor activity in PLXDC2-expressing cells. However, both compounds preferentially activate PLXDC1 over PLXDC2. A-Com-2 more strongly differentiates between the two receptors. All compound treatments were done for 1 day. Basal promotor activity of the PLXDC1-expressing cells is defined as 1. Fluorouracil (FU), a chemotherapy drug that kills dividing cells by apoptosis, do not activate this promotor.

FIG. 19 shows activation of PLXDC1 by antibodies. PLXDC1-activating antibodies (A-TEM7-Ab-1 and A-TEM7-Ab-2) activate the promotor activity in PLXDC1 cells, but not in cells without PLXDC1. Basal promotor activity of the PLXDC1-expressing cells is defined as 1. The basal activity of PLXDC1-expressing cells (Control) are higher than cells without PLXDC1, consistent promotor activation by the ectopic expression of the receptor without ligands.

FIG. 20A-B show killing of PLXDC1-expressing endothelial cells by PLXDC1-activating small molecules and antibodies. A. Visualization of the killing human PLXDC1-expressing endothelial cells by PLXDC1-activating small molecule (compound). The top three pictures on represent control cells and the lower three pictures represent compound-treated cells, showing light microscopy picture (left), live cell (middle) and dead cell staining (right). Live cells are stained using Fluorescein diacetate (green signal) and dead cells are stained using propidium iodide (red signal). B. Quantitation of the killing of human PLXDC1-expressing endothelial cells by PLXDC1-activating small molecules (A-Compound-1 and A-Compound-2) and antibodies (A-TEM7-Ab-1 and A-TEM7-Ab-2). Incubation time of the compounds and antibodies is 24 hours. Cell survival of the control cells is defined as 100%.

FIG. 21A-C show killing of human tumor endothelial cells expressing human PLXDC1 by receptor-activating antibodies against PLXDC1. The pictures represent one day after IgG addition and the right pictures represent 8 days after IgG addition. A. Incubation of human lung tumor endothelial cells with control IgG (500 nM) does not lead to the death of tumor endothelial cells expressing PLXDC1 (green signal). B. Incubation of human lung tumor endothelial cells with PLXDC1-activating IgG A-TEM7-Ab-1 (500 nM) leads to the death of tumor endothelial cells expressing PLXDC1 (green signal), as evident by comparing the tumor endothelial cell signal between day 1 and day 8. C. Incubation of human lung tumor endothelial cells with an independent PLXDC1-activativing IgG A-TEM7-Ab-2 (500 nM) also leads to the death of tumor endothelial cells expressing PLXDC1 (green signal), as evident by comparing the tumor endothelial cell signal between day 1 and day 8.

FIG. 22A-D show that PLXDC1-activating compound specifically suppresses pathogenic blood vessels in vivo without affecting healthy blood vessels in ischemia-induced retinopathy. A. Upper graph: Schematic diagram of the experimental design for ischemia-induced retinopathy. The high oxygen environment caused blood vessel loss (vaso-obliteration). In room air, loss of vessels triggered abnormal angiogenesis that generated pathogenic blood vessels on the top of the retina (marked in yellow in D). Treatment was applied during the return to room air by subcutaneous injection. Lower graph: quantitation of healthy blood vessels, vaso-obliteration and pathogenic blood vessels between the control (n=10) and treated retinas (n=10). Treatment by PLXDC1-activating compound (A-Compound-1) highly suppressed pathogenic blood vessels (two asterisks) while improving the amount of healthy blood vessels (one asterisk). B. Representative images of flat-mounted control retinas (upper two images) and retinas from compound treated mice (lower two images). Red signal is blood vessel marker. C. The same retinas in B with vaso-obliteration areas marked in white color. These images illustrate that compound-treated retinas went through vaso-obliteration like the control retinas. D. The same retinas in B with pathogenic blood vessels marked in yellow color. These images illustrate that compound-treated retinas have highly decreased pathogenic blood vessels as compared to the control retinas.

FIG. 23A-C show that PLXDC1-activating compound causes tumor shrinkage in vivo. Treatment was done at day 0 by bolus IV injection. A. Raw data of tumor growth curves of the mice in the control group. B. Raw data of tumor growth curves of the mice in the treatment group. C. Comparison of the combined growth data of the control group and the treatment group.

FIG. 24 shows tumor morphological changes on live animals due to the treatment by PLXDC1-activating compound. Pictures of the whole animals in the experiment described in FIG. 11 show tumor morphological and color changes on day 1 and day 3. Treatment was done at day 0. Tumors in the treatment groups becomes darker in color on day 1 due to the destruction of tumor blood vessels and accumulation of blood in the tumors. Tumors in the treatment groups start to become yellower in color on day 3, consistent with the onset of tumor necrosis due to the lack of tumor blood vessels.

FIG. 25A-B show tumor morphological changes on live animals due to the treatment by PLXDC1-activating compound. Pictures of the whole animals in the experiment described in FIG. 11 show tumor morphological and color changes on day 7. Treatment was done at day 0. While the tumors in the control group have grown to large sizes, tumors in the treatment groups have highly shrunk in size and become yellow in color.

FIG. 26A-B show morphological changes of dissected tumors due to the treatment by PLXDC1-activating compound. Pictures of the dissected tumors in the experiment described in FIG. 11 show tumor morphological and color changes on day 7. While the tumors in the control group are reddish in color, tumors in the treatment groups have highly shrunk in size and become yellow in color, consistent with the lack of tumor blood vessels and tumor necrosis.

FIG. 27A-B show that PLXDC1-activating antibodies bound to PLXDC1 independently of domain B. This property is in contrast to PEDF, which depends on domain B to bind to PLXDC1. A. Binding of PLXDC1-activating antibody (A-TEM7-Ab-1) to cells transfected with full length PLXDC1 (left picture), domain B-deleted PLXDC1 (middle picture) or untransfected control cells (right picture). The green (gray as shown) signal is the antibody binding signal. B. Binding of PLXDC1-activating antibody (A-TEM7-Ab-2) to cells transfected with full length PLXDC1 (left picture), domain B-deleted PLXDC1 (middle picture) or untransfected control cells (right picture). The green signal is the antibody binding signal.

### DETAILED DESCRIPTION

PLXDC1 and PLXDC2 are highly specifically expressed in the tumor blood vessels of diverse types of cancer (Beaty et al., 2007; Lu et al., 2007; Schwarze et al., 2005; St Croix et al., 2000; van Beijnum et al., 2009), and in the pathogenic blood vessels in diabetic retinopathy (Yamaji et al., 2008). This high enrichment is not present in healthy blood vessels (Beaty et al., 2007; Lu et al., 2007; Schwarze et al., 2005; St Croix et al., 2000; van Beijnum et al., 2009). High PLXDC1 expression has also been identified in choroidal neovascularization (pathogenic angiogenesis in age-related macular degeneration (AMD) and ischemia-induced retinopathy (pathogenic angiogenesis in retinopathy of prematurity).

Not all agents that bind to the extracellular domains of PLXDC1 or PLXDC2 can activate these receptors to kill endothelial cells. PLXDC1-binding protein, nidogen, for instance, does not have the therapeutic effect of killing pathogenic blood vessels. Anti-PLXDC1 antibodies have been developed as a potential anti-angiogenic therapy. In Bagley et al., Microvasc Res. 2011 November; 82(3):253-62, an anti-PLXDC1 antibody was identified that mediated antibody-dependent cellular cytotoxicity (ADCC) and phagocytosis. However, the antibodies in this approach depended on ADCC, and were not designed to activate PLXDC1 to kill endothelial cells without the immune system. Pigment Epithelium Derived Factor (PEDF) is a natural ligand for PLXDC1 and PLXDC2. However, binding of PEDF to PLXDC1 or PLXDC2 also does not provide the therapeutic effect as demonstrated in the ex vivo/in vitro models (Example 1).

The inventors have developed a novel array PLXDC1/PLXDC2 binding agents that induce the cell death in the endothelial cells that express PLXDC1/PLXDC2. This novel therapeutic modality is distinct from current anti-angiogenic strategies and current immunotherapy strategies that promote ADCC. As shown in the figures and examples of the application, these novel agents provided an effect that was different from other PLXDC1 or PLXDC2-targeting strategies, and also different from anti-angiogenic drugs such as anti-VEGF drugs or PEDF.

Examples 1-3 provide exemplary methods that were used for the screening and identification of cancer therapeutic agents, such as small molecule drugs and antibodies (Examples 1-7), that can effectively cause necrosis of and kill existing tumor blood vessels. In particular, suitable antibodies are identified based on the ability to preferentially bind to PLXDC1 or PLXDC2 that is activated by a small molecule activator. Because the small molecule activator promotes the activated conformation of PLXDC1 or PLXDC2, antibodies that preferentially bind to these proteins in the presence of the activator are expected to promote the activated conformation and are able to activate the PLXDC1 or PLXDC2 protein.

It is contemplated that PLXDC1 or PLXDC2 forms a dimer at the basal state. The small molecules and antibodies of the present disclosure, it is further contemplated, preferentially binds to amino acid residues of the protein that can interrupt or destabilize the receptor at the basal state. Such amino acid residues are not exposed on the surface of the protein when it is in the dimer/basal state. Preferably, such residues are on the dimer interface. They can also be residues that are only exposed when the dimer dissociates. In some embodiments, binding to the residue changes the conformation of the protein at the basal state.

The preferential binding to unexposed amino acid residues by the activating antibodies is evidenced by the antibody-screening method. The antibodies were screened for their ability to bind to the PLXDC protein in the presence of a small molecular activator, which promotes the activated conformation.

This also explains why not every ligand that binds to PLXDC1/PLXDC2 can activate it or kill endothelial cells expressing PLXDC1/PLXDC2 (e.g., tumor endothelial cells). As provided, nidogen, an extracellular matrix protein, binds to PLXDC1 but cannot activate PLXDC1 or kill endothelial cells expressing PLXDC1. Also, antibodies obtained via the conventional methods would not be able to activate PLXDC1/PLXDC2 as they can only bind to the exposed amino acid residues.

Identification of such antibodies and compounds was a surprise. To the best knowledge of the instant inventors, all existing antibody drugs are neutralizing antibodies or targeting antibodies. Neutralizing antibodies inhibit the ligand/receptor interaction, such as Humira (inhibiting TNF-α, a ligand), Avastin (inhibiting VEGF, a ligand), Herceptin (inhibiting HER2, a receptor), and Keytruda (inhibiting PD-1, a receptor). Targeting antibodies may exert their functions through mechanisms such as antibody-drug conjugates and antibody-dependent cell-mediated cytotoxicity (ADCC). No activating antibodies have been identified, in particular against single transmembrane cell-surface receptors like PLXDC1/PLXDC2.

These new therapeutic agents can trigger tumor blood vessel death through relevant downstream biological pathways. This demonstrates the effectiveness of the tissue models and the screening technologies developed herein. Such an effect of killing tumors by killing existing tumor blood vessels has not been reported. It is contemplated that these agents activate tumor blood vessel necrosis through activating PLXDC1 or PLXDC2. Therefore, the present disclosure provides new technology for effective tumor treatments. As PLXDC1 and PLXDC2 are present in many tumor types and angiogenesis is universally important for tumor development, this new technology presents a new direction for tumor treatment across tumor types.

### I. Definitions

As used herein, the terms “treat” or “treatment” refer to both therapeutic treatment and prophylactic or preventative measures, wherein the object is to prevent or slow down (lessen) an undesired physiological change or disorder, such as the progression of cancer or of a pathogenic blood vessel disorder. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The term “agent” or “therapeutic agent” is used herein to denote a chemical compound, a small molecule, a mixture of chemical compounds and/or a biological macromolecule (such as a nucleic acid, an antibody, an antibody fragment, a protein or a peptide). The activity of such agents may render them suitable as a “therapeutic agent” which is a biologically, physiologically, or pharmacologically active substance (or substances) that acts locally or systemically in a subject.

As used herein, the term “activator” refers to any agent that induces signal transduction changes in a cell through PLXDC1/PLXDC2.

The term “small molecule” is a term of the art and includes molecules that are less than about 2000 molecular weight or even less than about 1000 molecular weight. In certain embodiments, small molecules do not comprise a plurality of peptide bonds. In certain preferred embodiments, small molecules are not oligomeric. Exemplary small molecule compounds which can be screened for activity include, but are not limited to, peptides, peptidomimetics, nucleic acids, carbohydrates, small organic molecules (e.g., polyketides) (Cane et al. (1998) Science 282:63), and natural product extract libraries. In certain embodiments, the compounds are small, organic non-peptidic compounds.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on.

Pathogenic blood vessels are blood vessels that are not involved in the vascularization of normal organs but in the pathogenic tissues, such as the new hood vessels that drive vision diseases or the hood vessels in tumors that tumor depend on to survive. “Pathogenic blood vessel,” in some embodiments, refers to an existing blood vessel that may have vascularized a diseased tissue, for instance, a tumor. In other embodiments, a pathogenic blood vessel may be a blood vessel that is a newly formed blood vessel involved in disease onset and/or progression of, for example, cancer, diabetic retinopathy, age-related macular degeneration (AMD), retinopathy of prematurity and/or any other diseases having etiologies associated with angiogenesis.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more,” “at least one,” and “one or more than one.”

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

### II. Binding to PLXDC1/PLXDC2

The term “plexin domain-containing protein” or “PLXDC” refers to a small transmembrane receptor family that includes PLXDC1 and PLXDC2. PLXDC proteins have a large extracellular portion, a transmembrane domain, and share high sequence homology. Without limitation, the PLXDC protein can be PLXDC1 or PLXDC2.

PLXDC1 has a protein sequence as shown in NCBI Reference Sequence: NP_065138.2: MRGELWLLVLVLREAARALSPQPGAGHDEGPGSGWAAKGTVRGWNRRARESPGHVSEPDRTQ LSQDLGGGTLAMDTLPDNRTRVVEDNHSYYVSRLYGPSEPHSRELWVDVAEANRSQVKIHTILS NTHRQASRVVLSFDFPFYGHPLRQITIATGGFIFMGDVIHRMLTATQYVAPLMANFNPGYSDNST VVYFDNGTVFVVQWDHVYLQGWEDKGSFTFQAALHHDGRIVFAYKEIPMSVPEISSSQHPVKT GLSDAFMILNPSPDVPESRRRSIFEYHRIELDPSKVTSMSAVEFTPLPTCLQHRSCDACMSSDLTFN CSWCHVLQRCSSGFDRYRQEWMDYGCAQEAEGRMCEDFQDEDHDSASPDTSFSPYDGDLTTTS SSLFIDSLTTEDDTKLNPYAGGDGLQNNLSPKTKGTPVHLGTIVGIVLAVLLVAAIILAGIYINGHP TSNAALFFIERRPHHWPAMKFRSHPDHSTYAEVEPSGHEKEGFMEAEQC (SEQ ID NO:1), PLXDC2 has a sequence of NCBI Reference Sequence: NP_116201.7: MARFPKADLAAAGVMLLCHFFTDQFQFADGKPGDQILDWQYGVTQAFPHTEEEVEVDSHAYSH RWKRNLDFLKAVDTNRASVGQDSPEPRSFTDLLLDDGQDNNTQIEEDTDHNYYISRIYGPSDSAS RDLWVNIDQMEKDKVKIHGILSNTHRQAARVNLSFDFPFYGHFLREITVATGGFIYTGEVVHRM LTATQYIAPLMANFDPSVSRNSTVRYFDNGTALVVQWDHVHLQDNYNLGSFTFQATLLMDGRII FGYKEIPVLVTQISSTNHPVKVGLSDAFVVVHRIQQIPNVRRRTIYEYHRVELQMSKITNISAVEM TPLPTCLQFNRCGPCVSSQIGFNCSWCSKLQRCSSGFDRHRQDWVDSGCPEESKEKMCENTEPVE TSSRTTTTVGATTTQFRVLTTTRRAVTSQFPTSLPTEDDTKIALHLKDNGASTDDSAAEKKGGTL HAGLIIGILILVLIVATAILVTVYMYHHPTSAASIFFIERRPSRWPAMKFRRGSGHPAYAEVEPVGE KEGFIVSEQC (SEQ ID NO:2, isoform 1) or NP_001269665: MARFPKADLAAAGVMLLCHFFTDQFQFADGKPGDQILDWQYGVTQAFPHTEEEVEVDSHAYSH RWKRNLDFLKAVDTNRASVGQDSPEPRSFTDLLLDDGQDNNTQIERVNLSFDFPFYGHFLREITV ATGGFIYTGEVVHRMLTATQYIAPLMANFDPSVSRNSTVRYFDNGTALVVQWDHVHLQDNYNL GSFTFQATLLMDGRIIFGYKEIPVLVTQISSTNHPVKVGLSDAFVVVHRIQQIPNVRRRTIYEYHRV ELQMSKITNISAVEMTPLPTCLQFNRCGPCVSSQIGFNCSWCSKLQRCSSGFDRHRQDWVDSGCP EESKEKMCENTEPVETSSRTTTTVGATTTQFRVLTTTRRAVTSQFPTSLPTEDDTKIALHLKDNGA STDDSAAEKKGGTLHAGLIIGILILVLIVATAILVTVYMYHHPTSAASIFFIERRPSRWPAMKFRRG SGHPAYAEVEPVGEKEGFIVSEQC (SEQ ID NO:3, isoform 2). Their domain structures are shown in Tables 1 and 2 below.

TABLE 1
Alignment and rough domain structures of the PLXDC proteins

| | | |
|---|---|---|
| PLXDC1 | ---------MRGELWLLVLVLREAARALSPQPGAGHDEGPGSGWA--------------- | 36 |
| PLXDC2_1 | MARFPKADLAAAGVMLLCHFFTDQFQFADGKPGD-----QILDWQYGVTQAFPHTEEEVE | 55 |
| PLXDC2_2 | MARFPKADLAAAGVMLLCHFFTDQFQFADGKPGD-----QILDWQYGVTQAFPHTEEEVE | 55 |
| | _________________________________ | |
| | Domain A | |
| PLXDC1 | --AKGTVRGWNRRARESPGHVSEPDRTQLSQDLG----GGTLAMDTLPDNRTR-VVEDNH | 89 |
| PLXDC2_1 | VDSHAYSHRWKRNLDFLK--AVDTNRASVGQDSPEPRSFTDLLLDDGQDNNTQIEEDTDH | 113 |
| PLXDC2_2 | VDSHAYSHRWKRNLDFLK--AVDTNRASVGQDSPEPRSFTDLLLDDGQDNNTQIE----- | 108 |
| | ____________________________________________________________ | |
| | Domain A | |
| PLXDC1 | SYYVSRLYGPSEPHSRELWVDVAEANRSQVKIHTILSNTHRQASRVVLSFDFPFYGHPLR | 149 |
| PLXDC2_1 | NYYISRIYGPSDSASRDLWVNIDQMEKDKVKIRGILSNTHRQAARVNLSFDFPFYGHFLR | 173 |
| PLXDC2_2 | --------------------------------------------RVNLSFDFPFYGHFLR | 124 |
| | ______________________________________====================== | |
| | Domain A Domain B | |
| PLXDC1 | QITIATGGFIFMGDVIHRMLTATQYVAPLMANFNPGYSDNSTVVYFDNGTVFVVQWDHVY | 209 |
| PLXDC2_1 | EITVATGGFIYTGEVVHRMLTATQYIAPLMANFDPSVSRNSTVRYFDNGTALVVQWDHVH | 233 |
| PLXDC2_2 | EITVATGGFIYTGEVVHRMLTATQYIAPLMANFDPSVSRNSTVRYFDNGTALVVQWDHVH | 184 |
| | ============================================================ | |
| | Domain B | |
| PLXDC1 | LQGWEDKGSFTFQAALHHDGRIVFAYKEIPMSVPEISSSQHPVKTGLSDAFMILNPSPDV | 269 |
| PLXDC2_1 | LQDNYNLGSFTFQATLLMDGRIIFGYKEIPVLVTQISSTNHPVKVGLSDAFVVVHRIQQI | 293 |
| PLXDC2_2 | LQDNYNLGSFTFQATLLMDGRIIFGYKEIPVLVTQISSTNHPVKVGLSDAFVVVHRIQQI | 244 |
| | =================================\hat{ }\hat{ }\hat{ }\hat{ }\hat{ }\hat{ }\hat{ }\hat{ }\hat{ }\hat{ }\hat{ }\hat{ }\hat{ }\hat{ }\hat{ }\hat{ }\hat{ }\hat{ }\hat{ }\hat{ }\hat{ }\hat{ }\hat{ }\hat{ }\hat{ }\hat{ }\hat{ } | |
| | Domain B Domain C | |
| PLXDC1 | PESRRRSIFEYHRIELDPSKVTSMSAVEFTPLPTCLQHRSCDACMSSDLTFNCSWCHVLQ | 329 |
| PLXDC2_1 | PNVRRRTIYEYHRVELQMSKITNISAVEMTPLPTCLQFNRCGPCVSSQIGFNCSWCSKLQ | 353 |
| PLXDC2_2 | PNVRRRTIYEYHRVELQMSKITNISAVEMTPLPTCLQFNRCGPCVSSQIGFNCSWCSKLQ | 304 |
| | ^^^^^^^^^^^^^^^^^^^^^^^~~~~~~~~~~~~~~~~~~~~~~~~~~~~~~~~~~~~~ | |
| | Domain C Domain D | |
| PLXDC1 | RCSSGFDRYRQEWMDYGCAQEAEGRMCEDFQDEDHDSASPDT------SFSPYDGDLTTT | 383 |
| PLXDC2_1 | RCSSGFDRHRQDWVDSGCPEESKEKMCENTEPVETSSRTTTTVGATTTQFRVLT-TTRRA | 412 |
| PLXDC2_2 | RCSSGFDRHRQDWVDSGCPEESKEKMCENTEPVETSSRTTTTVGATTTQFRVLT-TTRRA | 363 |
| | ~~~~~~~~~~~~~~~~~~~~~~~~~~~~~~****************************** | |
| | Domain D Domain E | |
| PLXDC1 | SSSLFIDSLTTEDDTKLNPYAGGDGLQ-NNLSPKTKGTPVHLGTIVGIVLAVLLVAAIIL | 442 |
| PLXDC2_1 | VTSQFPTSLPTEDDTKIALHLKDNGASTDDSAAEKKGGTLHAGLIIGILILVLIVATAIL | 472 |
| PLXDC2_2 | VTSQFPTSLPTEDDTKIALHLKDNGASTDDSAAEKKGGTLHAGLIIGILILVLIVATAIL | 423 |
| | ********************************************================ | |
| | Domain E Transmembrane | |
| PLXDC1 | AGIYINGHPTSNAALFFIERRPHHWPAMKFRSHPDHSTYAEVEPSGHEKEGFMEAEQC | 500 |
| PLXDC2_1 | VTVYMYHHPTSAASIFFIERRPSRWPAMKFRRGSGHPAYAEVEPVG-EKEGFIVSEQC | 529 |
| PLXDC2_2 | VTVYMYHHPTSAASIFFIERRPSRWPAMKFRRGSGHPAYAEVEPVG-EKEGFIVSEQC | 480 |
| | =====_____________________________________________________ | |
| | Intracellular | |

TABLE 2
Listing of domains and locations

| Protein (SEQ ID NO:) | Domain | Amino acid residues |
|---|---|---|
| PLXDC1 | Domain A | 19-127 |
| (SEQ ID NO: 1) | Domain B | 128-242 |
| | Domain C | 243-292 |
| | Domain D | 293-359 |
| | Domain E | 360-427 |
| PLXDC2 isoform 1 | Domain A | 31-151 |
| (SEQ ID NO: 2) | Domain B | 152-266 |
| | Domain C | 267-316 |
| | Domain D | 317-383 |
| | Domain E | 384-454 |
| PLXDC2 isoform 2 | Domain A | 31-108 |
| (SEQ ID NO: 3) | Domain B | 109-207 |
| | Domain C | 208-267 |
| | Domain D | 268-334 |
| | Domain E | 335-405 |

A. Modulators of PLXDC1 and PLXDC2

In some aspects, provided herein are methods of treating cancer, as well as other diseases and disorder characterized with pathogenic blood vessels expressing PLXDC1 or PLXDC2, by activating PLXDC1 and/or PLXDC2 proteins in a subject (e.g., in a tumor blood vessel in a subject with cancer). In some embodiments, activating PLXDC1 and/or PLXDC2 receptors in a subject comprises administering an agent and/or activator described herein directly to the subject (e.g., by administering the activator and/or agent to the subject locally or systemically). In certain embodiments, the methods provided herein relate to activating PLXDC1 and/or PLXDC2 on the pathogenic blood vessel cells by contacting the cells with an agent disclosed herein.

In some aspects, provided herein are methods of treating cancer or inducing necrosis (e.g., coagulative necrosis) in a subject, comprising administering to the subject an agent that increases the activity of PLXDC1 and/or PLXDC2 on tumor blood vessel cells. In some embodiments, the activator and/or agent is a small molecule disclosed herein (e.g., a molecule of Formula I). Activators of PLXDC1 and/or PLXDC2 may be a PLXDC1 and/or PLXDC2 ligand, such as a peptide described herein. Other activators include small molecules and/or an antibody (e.g., an antibody disclosed herein). In some embodiments, the agent is administered locally or systemically. In some embodiments, the agent and/or activator may be administered locally to the subject's tumor or tissue comprising cancerous cells.

In some embodiments, a therapeutic agent of the present disclosure is able to bind to at least Domain A of PLXDC1 (amino acids 19-127 of SEQ ID NO:1). In some embodiments, a therapeutic agent of the present disclosure is able to bind to at least Domain B of PLXDC1 (amino acids 128-242 of SEQ ID NO:1). In some embodiments, a therapeutic agent of the present disclosure is able to bind to at least Domain C of PLXDC1 (amino acids 243-292 of SEQ ID NO:1). In some embodiments, a therapeutic agent of the present disclosure is able to bind to at least Domain D of PLXDC1 (amino acids 293-359 of SEQ ID NO:1). In some embodiments, a therapeutic agent of the present disclosure is able to bind to at least Domain E of PLXDC1 (amino acids 360-427 of SEQ ID NO:1).

In some embodiments, a therapeutic agent of the present disclosure is able to bind to at least Domain E of PLXDC1 and also binds to Domain D of PLXDC1. In some embodiments, a therapeutic agent of the present disclosure is able to bind to at least Domain E of PLXDC1 and also binds to Domain C of PLXDC1. In some embodiments, a therapeutic agent of the present disclosure is able to bind to at least Domain E of PLXDC1 and also binds to Domain B of PLXDC1. In some embodiments, a therapeutic agent of the present disclosure is able to bind to at least Domain E of PLXDC1 and also binds to Domain A of PLXDC1.

In some embodiments, a therapeutic agent of the present disclosure is able to bind to at least Domain D of PLXDC1 and also binds to Domain E of PLXDC1. In some embodiments, a therapeutic agent of the present disclosure is able to bind to at least Domain D of PLXDC1 and also binds to Domain C of PLXDC1. In some embodiments, a therapeutic agent of the present disclosure is able to bind to at least Domain D of PLXDC1 and also binds to Domain B of PLXDC1. In some embodiments, a therapeutic agent of the present disclosure is able to bind to at least Domain D of PLXDC1 and also binds to Domain A of PLXDC1.

In some embodiments, a therapeutic agent of the present disclosure is able to bind to at least Domain C of PLXDC1 and also binds to Domain E of PLXDC1. In some embodiments, a therapeutic agent of the present disclosure is able to bind to at least Domain C of PLXDC1 and also binds to Domain D of PLXDC1. In some embodiments, a therapeutic agent of the present disclosure is able to bind to at least Domain C of PLXDC1 and also binds to Domain B of PLXDC1. In some embodiments, a therapeutic agent of the present disclosure is able to bind to at least Domain C of PLXDC1 and also binds to Domain A of PLXDC1.

In some embodiments, a therapeutic agent of the present disclosure is able to bind to at least Domain B of PLXDC1 and also binds to Domain E of PLXDC1. In some embodiments, a therapeutic agent of the present disclosure is able to bind to at least Domain B of PLXDC1 and also binds to Domain D of PLXDC1. In some embodiments, a therapeutic agent of the present disclosure is able to bind to at least Domain B of PLXDC1 and also binds to Domain C of PLXDC1. In some embodiments, a therapeutic agent of the present disclosure is able to bind to at least Domain B of PLXDC1 and also binds to Domain A of PLXDC1.

In some embodiments, a therapeutic agent of the present disclosure is able to bind to at least Domain A of PLXDC1 and also binds to Domain E of PLXDC1. In some embodiments, a therapeutic agent of the present disclosure is able to bind to at least Domain A of PLXDC1 and also binds to Domain D of PLXDC1. In some embodiments, a therapeutic agent of the present disclosure is able to bind to at least Domain A of PLXDC1 and also binds to Domain C of PLXDC1. In some embodiments, a therapeutic agent of the present disclosure is able to bind to at least Domain A of PLXDC1 and also binds to Domain B of PLXDC1.

In some embodiments, the therapeutic agent of the present disclosure does not bind to Domain B of PLXDC1.

In some embodiments, a therapeutic agent of the present disclosure is able to bind to at least Domain A of PLXDC2 (amino acids 31-151 of SEQ ID NO:2 or amino acids 31-108 of SEQ ID NO:3). In some embodiments, a therapeutic agent of the present disclosure is able to bind to at least Domain B of PLXDC2 (amino acids 152-266 of SEQ ID NO:2 or amino acids 109-207 of SEQ ID NO:3). In some embodiments, a therapeutic agent of the present disclosure is able to bind to at least Domain C of PLXDC2 (amino acids 267-316 of SEQ ID NO:2 or amino acids 208-267 of SEQ ID NO:3). In some embodiments, a therapeutic agent of the present disclosure is able to bind to at least Domain D of PLXDC2 (amino acids 317-383 of SEQ ID NO:2 or amino acids 268-334 of SEQ ID NO:3). In some embodiments, a therapeutic agent of the present disclosure is able to bind to at least Domain E of PLXDC2 (amino acids 384-454 of SEQ ID NO:2 or amino acids 335-405 of SEQ ID NO:3).

In some embodiments, a therapeutic agent of the present disclosure is able to bind to at least Domain E of PLXDC2 and also binds to Domain D of PLXDC2. In some embodiments, a therapeutic agent of the present disclosure is able to bind to at least Domain E of PLXDC2 and also binds to Domain C of PLXDC2. In some embodiments, a therapeutic agent of the present disclosure is able to bind to at least Domain E of PLXDC2 and also binds to Domain B of PLXDC2. In some embodiments, a therapeutic agent of the present disclosure is able to bind to at least Domain E of PLXDC2 and also binds to Domain A of PLXDC2.

In some embodiments, a therapeutic agent of the present disclosure is able to bind to at least Domain D of PLXDC2 and also binds to Domain E of PLXDC2. In some embodiments, a therapeutic agent of the present disclosure is able to bind to at least Domain D of PLXDC2 and also binds to Domain C of PLXDC2. In some embodiments, a therapeutic agent of the present disclosure is able to bind to at least Domain D of PLXDC2 and also binds to Domain B of PLXDC2. In some embodiments, a therapeutic agent of the present disclosure is able to bind to at least Domain D of PLXDC2 and also binds to Domain A of PLXDC2.

In some embodiments, a therapeutic agent of the present disclosure is able to bind to at least Domain C of PLXDC2 and also binds to Domain E of PLXDC2. In some embodiments, a therapeutic agent of the present disclosure is able to bind to at least Domain C of PLXDC2 and also binds to Domain D of PLXDC2. In some embodiments, a therapeutic agent of the present disclosure is able to bind to at least Domain C of PLXDC2 and also binds to Domain B of PLXDC2. In some embodiments, a therapeutic agent of the present disclosure is able to bind to at least Domain C of PLXDC2 and also binds to Domain A of PLXDC2.

In some embodiments, a therapeutic agent of the present disclosure is able to bind to at least Domain B of PLXDC2 and also binds to Domain E of PLXDC2. In some embodiments, a therapeutic agent of the present disclosure is able to bind to at least Domain B of PLXDC2 and also binds to Domain D of PLXDC2. In some embodiments, a therapeutic agent of the present disclosure is able to bind to at least Domain B of PLXDC2 and also binds to Domain C of PLXDC2. In some embodiments, a therapeutic agent of the present disclosure is able to bind to at least Domain B of PLXDC1 and also binds to Domain A of PLXDC2.

In some embodiments, a therapeutic agent of the present disclosure is able to bind to at least Domain A of PLXDC2 and also binds to Domain E of PLXDC2. In some embodiments, a therapeutic agent of the present disclosure is able to bind to at least Domain A of PLXDC2 and also binds to Domain D of PLXDC2. In some embodiments, a therapeutic agent of the present disclosure is able to bind to at least Domain A of PLXDC2 and also binds to Domain C of PLXDC2. In some embodiments, a therapeutic agent of the present disclosure is able to bind to at least Domain A of PLXDC2 and also binds to Domain B of PLXDC2.

In some embodiments, the therapeutic agent of the present disclosure does not bind to Domain B of PLXDC2.

In some embodiments, the therapeutic agent of the present disclosure binds to PLXDC1 or PLXDC2 more strongly in the presence of a PLXDC1 or PLXDC2 activator, such as those disclosed herein, as compared to in the absence of such an activator. In some embodiments, the difference is at least 10%, 20%, 50%, 100%, 2.5 fold, 3 fold, 4 fold, 5 fold or 10 fold.

In some embodiments, the binding of a therapeutic agent of the present disclosure to PLXDC1 or PLXDC2 is at least 5 times, or at least 10, 15, 20, 50, 100, 1000, 10⁴, 10⁵, 10⁶, 10⁷, or 10⁸ times (or any derivable range therein) stronger than PEDF.

In some embodiments, the binding of a therapeutic agent of the disclosure, such as the compound and antibody modulators of PLXDC1 and/or PLXDC2 receptors described herein activate intracellular signaling pathways upon binding to PLXDC1 and/or PLXDC2. In some embodiments, a therapeutic agent of the disclosure inhibits dimerization of a PLXDC1 and/or PLXDC2 protein, such as homo or heterodimerization. The therapeutic agents of the disclosure may directly inhibit dimerization by directly binding to one or more portion(s) of the protein involved in dimerization or by binding and changing the conformation of the protein so that dimerization is reduced or eliminated.

In some embodiments, the agent binds to, binds at least, or binds at most 1, 2, 3, 4, 5, 6, 7, 8, 9, 10, 11, 12, 13, 14, 15, 16, 17, 18, 19, or 20 (or any derivable range therein) amino acid residues selected from the amino acid residue at position(s) 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, or 529 (or any derivable range therein) of a PLXDC1 or PLXDC2 polypeptide, such as those exemplified by any of SEQ ID NOS:1-3. It is also specifically contemplated that such agent may be excluded in any embodiment disclosed herein. In some embodiments, the binding of the agent to PLXDC1 or PLXDC2 or to a certain domain, epitope, or amino acid residue(s) may be with a K_{D} of at least, at most, or about 10⁻⁴, 10⁻⁵, 10⁻⁶, 10⁻⁷, 10⁻⁸, 10⁻⁹, 10⁻¹⁰, 10⁻¹¹, 10⁻¹², 10⁻¹³, 10⁻¹⁴, or 10⁻⁻¹⁵ M (or any derivable range therein). In some embodiments, the agent binds to the monomeric form of PLXDC1 and/or PLXDC2 with greater affinity than the dimeric form of the receptor. For example, the K_{D} for the monomeric form may be less than about 10⁻⁴, 10⁻⁵, 10⁻⁶, 10⁻⁷, 10⁻⁸, 10⁻⁹, 10⁻¹⁰, 10⁻¹¹, 10⁻¹², 10⁻⁻¹³, 10⁻⁻¹⁴, or 10⁻⁻¹⁵ M (or any derivable range therein), and the K_{D} for the dimeric form may be greater than about 10⁻¹, 10⁻², 10⁻³, 10⁻⁴, 10⁻⁵, or 10⁻⁻⁶ M (or any derivable range therein).

In some embodiments, the antibody is one that activates PLXDC1 and/or PLXDC2 in an assay as demonstrated in Example 5. In some embodiments, the antibody is one that demonstrates an efficacy of at least 60, 65, 70, 75, 80, 85, 90, 95, or 100% cell death (or any derivable range therein) in an assay as demonstrated in Example 6 or 7.

In some embodiments, the binding of a therapeutic agent of the present disclosure to a plexin domain-containing protein is able to cause activation of one or more genes in the tumor endothelial cell leading to tumor blood vessel death and tumor necrosis Non-limiting examples of such genes include ADAM17 (ADAM metallopeptidase domain 17), BAG4 (BCL2 associated athanogene 4), BIRC2 (baculoviral IAP repeat containing 2), BIRC3 (baculoviral IAP repeat containing 3), CASP8 (caspase 8), CAVI (caveolin 1), CHUK (component of inhibitor of nuclear factor kappa B kinase complex), CYLD (CYLD lysine 63 deubiquitinase), FADD (Fas associated via death domain), IKBKB (inhibitor of nuclear factor kappa B kinase subunit beta), IKB KG (inhibitor of nuclear factor kappa B kinase regulatory subunit gamma), ITCH (itchy E3 ubiquitin protein ligase), MADD (MAP kinase activating death domain), MAP2K3 (mitogen-activated protein kinase kinase 3), MAP2K7 (mitogen-activated protein kinase kinase 7), MAP3K1 (mitogen-activated protein kinase kinase kinase 1), MAP3K3 (mitogen-activated protein kinase kinase kinase 3), MAP3K5 (mitogen-activated protein kinase kinase kinase 5), MAP3K7 (mitogen-activated protein kinase kinase kinase 7), MAP4K2 (mitogen-activated protein kinase kinase kinase kinase 2), MAP4K3 (mitogen-activated protein kinase kinase kinase kinase 3), MAP4K4 (mitogen-activated protein kinase kinase kinase kinase 4), MAP4K5 (mitogen-activated protein kinase kinase kinase kinase 5), NFκB1 (nuclear factor kappa B subunit 1), NRK (Nik related kinase), NSMAF (neutral sphingomyelinase activation associated factor), PRKCI (protein kinase C iota), PRKCZ (protein kinase C zeta), RACK1 (receptor for activated C kinase 1), RBCK1 (RANBP2-type and C3HC4-type zinc finger containing 1), RELA (RELA proto-oncogene, NF-κB subunit), RFFL (ring finger and FYVE like domain containing E3 ubiquitin protein ligase), RIPK1 (receptor interacting serine/threonine kinase 1), RNF11 (ring finger protein 11), RNF31 (ring finger protein 31), SHARPIN (SHANK associated RH domain interactor), SMPD1 (sphingomyelin phosphodiesterase 1), SMPD2 (sphingomyelin phosphodiesterase 2), SQSTM1 (sequestosome 1), STAT1 (signal transducer and activator of transcription 1), TAB1 (TGF-beta activated kinase 1 (MAP3K7) binding protein 1), TAB2 (TGF-beta activated kinase 1 (MAP3K7) binding protein 2), TAB3 (TGF-beta activated kinase 1 (MAP3K7) binding protein 3), TAX1BP1 (Taxi binding protein 1), TNF (tumor necrosis factor), TNFAIP3 (TNF alpha induced protein 3), TNFRSF1A (TNF receptor superfamily member 1A), TNFRSF1B (TNF receptor superfamily member 1B), TNIK (TRAF2 and NCK interacting kinase), TRADD (TNFRSF1A associated via death domain), TRAF1 (TNF receptor associated factor 1), TRAF2 (TNF receptor associated factor 2), TRAF5 (TNF receptor associated factor 5), and TXN (thioredoxin).

### III. Compounds of Formula I

A. Chemical Definitions

As used in the present specification, the following words, phrases and symbols are generally intended to have the meanings as set forth below, except to the extent that the context in which they are used indicates otherwise.

A dash (“-”) that is not between two letters or symbols is used to indicate a point of attachment for a substituent. For example, —C(O)NH₂ is attached through the carbon atom. A dash at the front or end of a chemical group is a matter of convenience; chemical groups may be depicted with or without one or more dashes without losing their ordinary meaning. A wavy line or a dashed line drawn through a line in a structure indicates a specified point of attachment of a group. Unless chemically or structurally required, no directionality or stereochemistry is indicated or implied by the order in which a chemical group is written or named.

The term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, etc. C₀ alkyl indicates a hydrogen where the group is in a terminal position, a bond if internal. The terms “C_{2-y}alkenyl” and “C_{2-y}alkynyl” refer to substituted or unsubstituted unsaturated aliphatic groups analogous in length and possible substitution to the alkyls described above, but that contain at least one double or triple bond respectively. The singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

“Alkyl” refers to an unbranched or branched saturated hydrocarbon chain. In some embodiments, alkyl has the indicated number of carbon atoms. In some embodiments, alkyl has 1 to 40 carbon atoms (i.e., C₁₋₄₀ alkyl), 1 to 30 carbon atoms (i.e., C₁₋₃₀ alkyl), 10 to 30 carbon atoms (i.e., C₁₀₋₃₀ alkyl), 1 to 20 carbon atoms (i.e., C₁₋₂₀ alkyl), 1 to 12 carbon atoms (i.e., C₁₋₁₂ alkyl), 1 to 8 carbon atoms (i.e., C₁₋₈ alkyl), 1 to 6 carbon atoms (i.e., C₁₋₆ alkyl) or 1 to 4 carbon atoms (i.e., C₁₋₄ alkyl). Examples of alkyl groups include, e.g., methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl, 3-methylpentyl, octyl, nonyl, decyl, dodecyl, icosyl, docosyl, and tetradecyl. When an alkyl residue having a specific number of carbons is named by chemical name or identified by molecular formula, all positional isomers having that number of carbons may be encompassed; thus, for example, “butyl” includes n-butyl (i.e., —(CH₂)₃CH₃), sec-butyl (i.e., —CH(CH₃)CH₂CH₃), isobutyl (i.e., —CH₂CH(CH₃)₂) and tert-butyl (i.e., —C(CH₃)₃); and “propyl” includes n-propyl (i.e., —(CH₂)₂CH₃) and isopropyl (i.e., —CH(CH₃)₂).

Certain commonly used alternative chemical names may be used. For example, a divalent group such as a divalent “alkyl” group, a divalent “aryl” group, etc., may also be referred to as an “alkylene” group, an “arylene” group, respectively. Also, unless indicated explicitly otherwise, where combinations of groups are referred to herein as one moiety, e.g., arylalkyl or aralkyl, the last mentioned group contains the atom by which the moiety is attached to the rest of the molecule.

“Alkenyl” refers to an alkyl group containing at least one carbon-carbon double bond. In some embodiments, alkenyl has the indicated number of carbon atoms. In some embodiments, alkenyl has from 2 to 40 carbon atoms (i.e., C₂₋₄₀ alkenyl), 2 to 30 carbon atoms (i.e., C₂₋₃₀ alkenyl), 10 to 30 carbon atoms (i.e., C₁₀₋₃₀ alkenyl), 2 to 20 carbon atoms (i.e., C₂₋₂₀ alkenyl), 2 to 8 carbon atoms (i.e., C₂₋₈ alkenyl), 2 to 6 carbon atoms (i.e., C₂₋₆ alkenyl) or 2 to 4 carbon atoms (i.e., C₂₋₄ alkenyl). Examples of alkenyl groups include, e.g., ethenyl, propenyl, butadienyl (including 1,2-butadienyl and 1,3-butadienyl).

“Alkynyl” refers to an alkyl group containing at least one carbon-carbon triple bond. In some embodiments, alkynyl has the indicated number of carbon atoms. In some embodiments, alkynyl has from 2 to 40 carbon atoms (i.e., C₂₋₄₀ alkynyl), 2 to 30 carbon atoms (i.e., C₁₋₃₀ alkynyl), 10 to 30 carbon atoms (i.e., C₁₀₋₃₀ alkynyl), 2 to 20 carbon atoms (i.e., C₂₋₂₀ alkynyl), 2 to 8 carbon atoms (i.e., C₂₋₈ alkynyl), 2 to 6 carbon atoms (i.e., C₂₋₆ alkynyl) or 2 to 4 carbon atoms (i.e., C₂₋₄ alkynyl). The term “alkynyl” also includes those groups having one triple bond and one double bond.

“Alkoxy” refers to the group “alkyl-O—”. In some embodiments, alkoxy has from 1 to 40 carbon atoms (i.e., —O—C₁₋₄₀ alkyl), 1 to 30 carbon atoms (i.e., —O—C₁₋₃₀ alkyl), 10 to 30 carbon atoms (i.e., —O—C₁₀₋₃₀ alkyl, 1 to 20 carbon atoms (i.e., —O—C₁₋₂₀ alkyl), 1 to 12 carbon atoms (i.e., —O—C₁₋₁₂ alkyl), 1 to 8 carbon atoms (i.e., —O—C₁₋₈ alkyl), 1 to 6 carbon atoms (i.e., —O—C₁₋₆ alkyl) or 1 to 4 carbon atoms (i.e., —O—C₁₋₄ alkyl). Examples of alkoxy groups include, e.g., methoxy, ethoxy, n-propoxy, iso-propoxy, n-butoxy, tert-butoxy, sec-butoxy, n-pentoxy, n-hexoxy and 1,2-dimethylbutoxy.

“Alkenoxy” refers to the group “alkene-O—”. In some embodiments, alkenoxy has from 2 to 40 carbon atoms (i.e., —O—C₂₋₄₀ alkene), 2 to 30 carbon atoms (i.e., —O—C₂₋₃₀ alkene), 10 to 30 carbon atoms (i.e., —O—C₁₀₋₃₀ alkene), 2 to 20 carbon atoms (i.e., —O—C₂₋₂₀ alkene), 2 to 12 carbon atoms (i.e., —O—C₂₋₁₂ alkene), 2 to 8 carbon atoms (i.e., —O—C₂₋₈ alkene), 2 to 6 carbon atoms (i.e., —O—C₂₋₆ alkene) or 2 to 4 carbon atoms (i.e., —O—C₂₋₄ alkene).

“Alkynoxy” refers to the group “alkyne-O—”. In some embodiments, alkynoxy has from 1 to 40 carbon atoms (i.e., —O—C₂₋₄₀ alkyne), 2 to 30 carbon atoms (i.e., —O—C₂₋₃₀ alkene), 10 to 30 carbon atoms (i.e., —O—C₁₀₋₃₀ alkyne, 2 to 20 carbon atoms (i.e., —O—C₂₋₂₀ alkyne), 2 to 12 carbon atoms (i.e., —O—C₂₋₁₂ alkyne), 2 to 8 carbon atoms (i.e., —O—C₂₋₈ alkyne), 2 to 6 carbon atoms (i.e., —O—C₂₋₆ alkyne) or 2 to 4 carbon atoms (i.e., —O—C₂₋₄ alkyne).

“Amino” refers to the group —NR^{y}R^{z} wherein R^{y} and R^{z} are independently hydrogen, alkyl, alkenyl, alkynyl, cycloalkyl, heterocyclyl, aryl, heteroalkyl or heteroaryl; each of which may be optionally substituted, as defined herein.

“Aryl” refers to an aromatic carbocyclic group having a single ring (e.g., monocyclic) or multiple rings (e.g., bicyclic or tricyclic) including fused systems. In some embodiments, aryl has 6 to 20 ring carbon atoms (i.e., C₆₋₂₀ aryl), 6 to 12 ring carbon atoms (i.e., C₆₋₁₂ aryl), or 6 to 10 ring carbon atoms (i.e., C₆₋₁₀ aryl). Examples of aryl groups include, e.g., phenyl, naphthyl, fluorenyl and anthryl. Aryl, however, does not encompass or overlap in any way with heteroaryl defined below. If one or more aryl groups are fused with a heteroaryl, the resulting ring system is heteroaryl. If one or more aryl groups are fused with a heterocyclyl, the resulting ring system is heterocyclyl.

“Arylalkyl” or “Aralkyl” refers to the group “aryl-alkyl-”.

“Carboxyl ester” or “ester” refer to both —OC(O)R^{x} and —C(O)OR^{x}, wherein R^{x} is alkyl, alkenyl, alkynyl, cycloalkyl, heterocyclyl, aryl, heteroalkyl or heteroaryl; each of which may be optionally substituted, as defined herein.

“Carboxy” as used herein refers to —CO₂H, or a salt thereof. Exemplary counter ions which can be used include, but are not limited to, Na⁺, K⁺, Li⁺, NH₄⁺ and others described herein.

“Cycloalkyl” refers to a saturated or partially unsaturated cyclic alkyl group having a single ring or multiple rings including fused, bridged and spiro ring systems. The term “cycloalkyl” includes cycloalkenyl groups (i.e., the cyclic group having at least one double bond) and carbocyclic fused ring systems having at least one sp³ carbon atom (i.e., at least one non-aromatic ring). In some embodiments, cycloalkyl has from 3 to 20 ring carbon atoms (i.e., C₃₋₂₀ cycloalkyl), 3 to 12 ring carbon atoms (i.e., C₃₋₁₂ cycloalkyl), 3 to 10 ring carbon atoms (i.e., C₃₋₁₀ cycloalkyl), 3 to 8 ring carbon atoms (i.e., C₃₋₈ cycloalkyl), or 3 to 6 ring carbon atoms (i.e., C₃-6 cycloalkyl). Monocyclic groups include, for example, cyclopropyl, cyclobutyl, cyclopentyl, cyclohexyl, cycloheptyl and cyclooctyl. Polycyclic cycloalkyl refers to a cycloalkyl having at least two rings, which may be a fused, bridged or spiro ring system. Polycyclic groups include, for example, bicyclo[2.2.1]heptanyl, bicyclo[2.2.2]octanyl, adamantyl, norbornyl, decalinyl, 7,7-dimethyl-bicyclo[2.2.1]heptanyl and the like. “Spirocycloalkyl” refers to a polycyclic cycloalkyl group wherein at least two rings are linked together by one common atom, for example spiro[2.5]octanyl, spiro[4.5]decanyl, or spiro[5.5]undecanyl. Spirocycloalkyl may contain fused rings in the ring system, but not bridged rings. “Fused cycloalkyl” refers to a polycyclic cycloalkyl group wherein at least two rings are linked together by two common atoms wherein the two common atoms are connected through a covalent bond. Fused cycloalkyl does not contain any spiro or bridged rings in the ring system. “Bridged cycloalkyl” refers to a polycyclic cycloalkyl that contains a bridge—an alkylene (such as C₁₋₄ alkylene) group that connect two “bridgehead” atoms. Non-limiting examples of bridged cycloalkyl include bicyclo[2.2.1]heptanyl, bicyclo[2.2.2] octanyl, adamantyl, norbornyl, and 7,7-dimethyl-bicyclo[2.2.1]heptanyl. Bridged cycloalkyl may contain fused and/or spiro rings in the ring system. Further, the term cycloalkyl is intended to encompass any non-aromatic ring which may be fused to an aryl ring, regardless of the attachment to the remainder of the molecule.

“Halogen” or “halo” refers to atoms occupying group VIIA of the periodic table, such as fluoro, chloro, bromo or iodo.

“Haloalkyl” refers to an unbranched or branched alkyl group as defined above, wherein one or more (e.g., 1 to 6, 1 to 5 or 1 to 3) hydrogen atoms are replaced by a halogen. For example, where a residue is substituted with more than one halogen, it may be referred to by using a prefix corresponding to the number of halogen moieties attached. Dihaloalkyl and trihaloalkyl refer to alkyl substituted with two (“di”) or three (“tri”) halo groups, which may be, but are not necessarily, the same halogen. Examples of haloalkyl include, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl and the like.

“Haloalkoxy” refers to an alkoxy group as defined above, wherein one or more (e.g., 1 to 6, 1 to 5 or 1 to 3) hydrogen atoms are replaced by a halogen.

“Hydroxyalkyl” refers to an alkyl group as defined above, wherein one or more (e.g., 1 to 6, 1 to 5 or 1 to 3) hydrogen atoms are replaced by a hydroxy group. A non-limiting example of hydroxyalkyl is —(CH₂)₁₋₄—OH.

“Heteroalkyl” refers to an alkyl group in which one or more, but not all of the carbon atoms (and any associated hydrogen atoms) are each independently replaced with the same or different heteroatomic group, provided the point of attachment to the remainder of the molecule is through a carbon atom. The term “heteroalkyl” includes unbranched or branched saturated chain having carbon and heteroatoms. By way of example, 1, 2 or 3 carbon atoms may be independently replaced with the same or different heteroatomic group. Heteroatomic groups include, but are not limited to, —NR^{y}—, —O—, —S—, —S(O)—, —S(O)₂—, and the like, wherein R^{y} is hydrogen, alkyl, alkenyl, alkynyl, cycloalkyl, heterocyclyl, aryl, heteroalkyl or heteroaryl; each of which may be optionally substituted, as defined herein. Examples of heteroalkyl groups include, e.g., ethers (e.g., —CH₂OCH₃, —CH(CH₃)OCH₃, —CH₂CH₂OCH₃, —CH₂CH₂OCH₂CH₂OCH₃, etc.), thioethers (e.g., —CH₂SCH₃, —CH(CH₃)SCH₃, —CH₂CH₂SCH₃, —CH₂CH₂SCH₂CH₂SCH₃, etc.), sulfones (e.g., —CH₂S(O)₂CH₃, —CH(CH₃)S(O)₂CH₃, —CH₂CH₂S(O)₂CH₃, —CH₂CH₂S(O)₂CH₂CH₂OCH₃, etc.) and amines (e.g., —CH₂NR^{y}CH₃, —CH(CH₃)NR^{y}CH₃, —CH₂CH₂NR^{y}CH₃, —CH₂CH₂NR^{y}CH₂CH₂NR^{y}CH₃, etc., where R^{y} is hydrogen, alkyl, alkenyl, alkynyl, cycloalkyl, heterocyclyl, aryl, heteroalkyl, or heteroaryl; each of which may be optionally substituted, as defined herein). In some embodiments, heteroalkyl includes 1 to 10 carbon atoms (C₁ heteroalkyl), 1 to 8 carbon atoms (C₁₋₈ heteroalkyl), or 1 to 4 carbon atoms (C₁₋₄ heteroalkyl); and 1 to 3 heteroatoms, 1 to 2 heteroatoms, or 1 heteroatom.

“Heteroaryl” refers to an aromatic group having a single ring, multiple rings or multiple fused rings, with one or more ring heteroatoms independently selected from nitrogen, oxygen, and sulfur. As used herein, heteroaryl includes 1 to 20 ring carbon atoms (i.e., C₁₋₂₀ heteroaryl), 3 to 12 ring carbon atoms (i.e., C₃₋₁₂ heteroaryl), or 3 to 8 carbon ring atoms (i.e., C₃₋₈ heteroaryl), and 1 to 5 ring heteroatoms, 1 to 4 ring heteroatoms, 1 to 3 ring heteroatoms, 1 to 2 ring heteroatoms, or 1 ring heteroatom independently selected from nitrogen, oxygen and sulfur. In certain instances, heteroaryl includes 5-10 membered ring systems, 5-7 membered ring systems, or 5-6 membered ring systems, each independently having 1 to 4 ring heteroatoms, 1 to 3 ring heteroatoms, 1 to 2 ring heteroatoms, or 1 ring heteroatom independently selected from nitrogen, oxygen and sulfur. Examples of heteroaryl groups include, e.g., acridinyl, benzimidazolyl, benzothiazolyl, benzindolyl, benzofuranyl, benzothiazolyl, benzothiadiazolyl, benzonaphthofuranyl, benzoxazolyl, benzothienyl (benzothiophenyl), benzotriazolyl, benzo[4,6]imidazo[1,2-a]pyridyl, carbazolyl, cinnolinyl, dibenzofuranyl, dibenzothiophenyl, furanyl, isothiazolyl, imidazolyl, indazolyl, indolyl, indazolyl, isoindolyl, isoquinolyl, isoxazolyl, naphthyridinyl, oxadiazolyl, oxazolyl, 1-oxidopyridinyl, 1-oxidopyrimidinyl, 1-oxidopyrazinyl, 1-oxidopyridazinyl, phenazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, thiazolyl, thiadiazolyl, triazolyl, tetrazolyl and triazinyl. Examples of the fused-heteroaryl rings include, but are not limited to, benzo[d]thiazolyl, quinolinyl, isoquinolinyl, benzo[b]thiophenyl, indazolyl, benzo[d]imidazolyl, pyrazolo[1,5-a]pyridinyl and imidazo[1,5-a]pyridinyl, where the heteroaryl can be bound via either ring of the fused system. Any aromatic ring, having a single or multiple fused rings, containing at least one heteroatom, is considered a heteroaryl regardless of the attachment to the remainder of the molecule (i.e., through any one of the fused rings). Heteroaryl does not encompass or overlap with aryl as defined above.

“Heterocyclyl” refers to a saturated or partially unsaturated cyclic alkyl group, with one or more ring heteroatoms independently selected from nitrogen, oxygen and sulfur. The term “heterocyclyl” includes heterocycloalkenyl groups (i.e., the heterocyclyl group having at least one double bond), bridged-heterocyclyl groups, fused-heterocyclyl groups and spiro-heterocyclyl groups. A heterocyclyl may be a single ring or multiple rings wherein the multiple rings may be fused, bridged or spiro, and may comprise one or more (e.g., 1 to 3) oxo (—O⁻) or N-oxide (—O⁻) moieties. Any non-aromatic ring containing at least one heteroatom is considered a heterocyclyl, regardless of the attachment (i.e., can be bound through a carbon atom or a heteroatom). Further, the term heterocyclyl is intended to encompass any non-aromatic ring containing at least one heteroatom, which ring may be fused to an aryl or heteroaryl ring, regardless of the attachment to the remainder of the molecule. As used herein, heterocyclyl has 2 to 20 ring carbon atoms (i.e., C₂₋₂₀ heterocyclyl), 2 to 12 ring carbon atoms (i.e., C₂₋₁₂ heterocyclyl), 2 to 10 ring carbon atoms (i.e., C₂₋₁₀ heterocyclyl), 2 to 8 ring carbon atoms (i.e., C₂₋₈ heterocyclyl), 3 to 12 ring carbon atoms (i.e., C₃₋₁₂ heterocyclyl), 3 to 8 ring carbon atoms (i.e., C₃₋₈ heterocyclyl), or 3 to 6 ring carbon atoms (i.e., C₃-6 heterocyclyl); having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur or oxygen. In certain instances, heterocyclyl includes 3- to 10-membered heterocyclyl having 3-10 total ring atoms, 5- to 7-membered heterocyclyl having 5-7 total ring atoms, or 5- or 6-membered heterocyclyl having 5 or 6 total ring atoms. Examples of heterocyclyl groups include, e.g., azetidinyl, azepinyl, benzodioxolyl, benzo[b][1,4]dioxepinyl, 1,4-benzodioxanyl, benzopyranyl, benzodioxinyl, benzopyranonyl, benzofuranonyl, dioxolanyl, dihydropyranyl, hydropyranyl, thienyl[1,3]dithianyl, decahydroisoquinolyl, furanonyl, imidazolinyl, imidazolidinyl, indolinyl, indolizinyl, isoindolinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, oxiranyl, oxetanyl, phenothiazinyl, phenoxazinyl, piperidinyl, piperazinyl, 4-piperidonyl, pyrrolidinyl, pyrazolidinyl, quinuclidinyl, thiazolidinyl, tetrahydrofuryl, tetrahydropyranyl, trithianyl, tetrahydroquinolinyl, thiophenyl (i.e., thienyl), tetrahydropyranyl, thiomorpholinyl, thiamorpholinyl, 1-oxo-thiomorpholinyl and 1,1-dioxo-thiomorpholinyl. The term “heterocyclyl” also includes “spiroheterocyclyl” when there are at least two rings are linked together by one common atom. Examples of the spiro-heterocyclyl rings include, e.g., bicyclic and tricyclic ring systems, such as 2-oxa-7-azaspiro[3.5]nonanyl, 2-oxa-6-azaspiro[3.4]octanyl and 6-oxa-1-azaspiro[3.3]heptanyl. Examples of the fused-heterocyclyl rings include, but are not limited to, 1,2,3,4-tetrahydroisoquinolinyl, 4,5,6,7-tetrahydrothieno[2,3-c]pyridinyl, indolinyl and isoindolinyl, where the heterocyclyl can be bound via either ring of the fused system. Examples of heterocyclyl include sugar moieties such as glucose, mannose, allose, altrose, gulose, idose, galactose, and talose.

The terms “alkylthio” or “thioalkyl” as used herein refer to —S-alkyl, where the term alkyl is as defined herein.

The term “sulfonamido” as used herein refer to both —NR^{g}S(═O)₂R^{h} and —S(═O)₂NR^{g}R^{h}, wherein each of R^{g} and R^{h} is independently hydrogen, alkyl, alkenyl, alkynyl, alkoxy, thioalkyl, aryl, aryl-alkyl, cycloalkyl, cycloalkyl-alkyl, haloalkyl, heterocyclyl, heterocyclyl-alkyl, heteroaryl, or heteroaryl-alkyl, and further wherein each R^{g} and R^{h} may be optionally substituted, as defined herein.

The term “sulfinamido” as used herein refer to both —NR^{g}S(═O)R^{h} and —S(═O)NR^{g}R^{h}, wherein each of R^{g} and R^{h} is independently hydrogen, alkyl, alkenyl, alkynyl, alkoxy, thioalkyl, aryl, aryl-alkyl, cycloalkyl, cycloalkyl-alkyl, haloalkyl, heterocyclyl, heterocyclyl-alkyl, heteroaryl, or heteroaryl-alkyl, and further wherein each R^{g} and R^{h} may be optionally substituted, as defined herein.

The term “sulfoxide” or “sulfoxido” refers to the group —S(═O)—R^{g}, wherein R^{g} is hydrogen, alkyl, alkenyl, alkynyl, alkoxy, thioalkyl, aryl, aryl-alkyl, cycloalkyl, cycloalkyl-alkyl, haloalkyl, heterocyclyl, heterocyclyl-alkyl, heteroaryl, or heteroaryl-alkyl, and further wherein R^{g} may be optionally substituted, as defined herein.

The term “sulfonyl” refers to the group —S(O)₂—R^{g}, wherein R^{g} is hydrogen, alkyl, alkenyl, alkynyl, alkoxy, thioalkyl, aryl, aryl-alkyl, cycloalkyl, cycloalkyl-alkyl, haloalkyl, heterocyclyl, heterocyclyl-alkyl, heteroaryl, or heteroaryl-alkyl, and further wherein R^{g} may be optionally substituted, as defined herein. “Sugar moiety” refers to a monovalent radical of a sugar molecule, such as a monosaccharide molecule, including glucose (also known as dextrose), fructose, galactose, mannose, allose, altrose, gulose, idose, and talose. As used herein, a sugar moiety a heterocyclyl substituted with OH and/or hydoxyalkyl groups. However, it is understood that a sugar moiety can exist in a liner form as an alkyl substituted with oxo and OH groups.

The terms “optional” or “optionally” means that the subsequently described event or circumstance may or may not occur and that the description includes instances where said event or circumstance occurs and instances in which it does not. Also, the term “optionally substituted” refers to any one or more (e.g., 1 to 5 or 1 to 3) hydrogen atoms on the designated atom or group may or may not be replaced by a moiety other than hydrogen.

In certain embodiments, “substituted” includes any of the above alkyl, heteroalkyl, alkenyl, alkynyl, cycloalkyl, heterocyclyl, aryl or heteroaryl groups in which one or more (e.g., 1 to 5 or 1 to 3) hydrogen atoms are independently replaced with halo, cyano, nitro, azido, oxo, alkyl, alkenyl, alkynyl, haloalkyl, cycloalkyl, heterocyclyl, aryl, heteroaryl, —NR^{g}R^{h}, —C(NR^{g})R^{h}, —C(NR^{g})(NR^{h}₂), —NR^{g}C(═O)R^{h}, —NR^{g}C(═O)NR^{g}R^{h}, —NR^{g}C(═O)OR^{h}, —NR^{g}S(═O)₁₋₂R^{h}, —C(═O)R^{g}, —C(═O)OR^{g}, —OC(═O)OR^{g}, —OC(═O)R^{g}, —C(═O)NR^{g}R^{h}, —OC(═O)NR^{g}R^{h}, —OR^{g}, —SR^{g}, —S(═O)R^{g}, —S(═O)₂R^{g}, —OS(═O)₁₋₂R^{g}, —S(═O)₁₋₂OR^{g}, —NR^{g}S(═O)₁₋₂NR^{g}R^{h}, ═NSO₂R^{g}, ═NOR^{g}, —S(═O)₁₋₂NR^{g}R^{h}, —CR^{g}(═NOH), —NR^{g}C(═NR^{h})(NR^{h}R^{h}), —SF₅, —SCF₃ or —OCF₃. In certain embodiments, “substituted” also means any of the above groups in which one or more (e.g., 1 to 5 or 1 to 3) hydrogen atoms are replaced with —C(═O)R^{g}, —C(═O)OR^{g}, —C(═O)NR^{g}R^{h}, —CH₂SO₂R^{g}, or —CH₂SO₂NR^{g}R^{h}. In the foregoing, each of R^{g} and R^{h} is independently hydrogen, alkyl, alkenyl, alkynyl, alkoxy, thioalkyl, aryl, aralkyl, cycloalkyl, cycloalkyl-alkyl, haloalkyl, heterocyclyl, heterocyclyl-alkyl, heteroaryl, and/or heteroaryl-alkyl. In certain embodiments, “substituted” also means any of the above groups in which one or more (e.g., 1 to 5 or 1 to 3) hydrogen atoms are replaced with halo, hydroxy, alkyl, alkylhydroxy, or oxo groups.

Polymers or similar indefinite structures arrived at by defining substituents with further substituents appended ad infinitum (e.g., a substituted aryl having a substituted alkyl which is itself substituted with a substituted aryl group, which is further substituted by a substituted heteroalkyl group, etc.) are not intended for inclusion herein. Unless otherwise noted, the maximum number of serial substitutions in compounds described herein is three. For example, serial substitutions of substituted aryl groups with two other substituted aryl groups are limited to ((substituted aryl)substituted aryl)substituted aryl. Similarly, the above definitions are not intended to include impermissible substitution patterns (e.g., methyl substituted with 5 fluorines or heteroaryl groups having two adjacent oxygen ring atoms). Such impermissible substitution patterns are well known to the skilled artisan.

In certain embodiments, as used herein, the phrase “one or more” refers to one to five. In certain embodiments, as used herein, the phrase “one or more” refers to one to three.

Any compound or structure given herein, is also intended to represent unlabeled forms as well as isotopically labeled forms of the compounds. These forms of compounds may also be referred to as “isotopically enriched analogs.” Isotopically labeled compounds have structures depicted herein, except that one or more atoms are replaced by an atom having a selected atomic mass or mass number. Examples of isotopes that can be incorporated into the disclosed compounds include isotopes of hydrogen, carbon, nitrogen, oxygen, phosphorous, fluorine, chlorine and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Various isotopically labeled compounds of the present disclosure, for example those into which radioactive isotopes such as ³H or ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of subjects.

The term “isotopically enriched analogs” includes “deuterated analogs” of compounds described herein in which one or more hydrogens is/are replaced by deuterium, such as a hydrogen on a carbon atom. Such compounds may exhibit increased resistance to metabolism and are thus useful for increasing the half-life of any compound when administered to a mammal, particularly a human See, for example, Foster, “Deuterium Isotope Effects in Studies of Drug Metabolism,” Trends Pharmacol. Sci. 5(12):524-527 (1984). Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F, ³H, ¹¹C labeled compound may be useful for PET or SPECT or other imaging studies. Isotopically labeled compounds of this disclosure can generally be prepared by carrying out the procedures disclosed in the schemes or in the examples and preparations described below by substituting a readily available isotopically labeled reagent for a non-isotopically labeled reagent.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when an atom is represented by its name or letter symbol, such as, H, C, O, or N, it is understood that the atom has its natural abundance isotopic composition. For example, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium.

In many cases, the compounds of this disclosure are capable of forming acid and/or base salts by virtue of the presence of amino and/or carboxyl groups or groups similar thereto.

Provided also are a pharmaceutically acceptable salt, isotopically enriched analog, deuterated analog, stereoisomer, and mixture of stereoisomers of the compounds described herein. “Pharmaceutically acceptable” or “physiologically acceptable” refer to compounds, salts, compositions, dosage forms and other materials which are useful in preparing a pharmaceutical composition that is suitable for veterinary or human pharmaceutical use.

The term “pharmaceutically acceptable salt” of a given compound refers to salts that retain the biological effectiveness and properties of the given compound and which are not biologically or otherwise undesirable. “Pharmaceutically acceptable salts” or “physiologically acceptable salts” include, for example, salts with inorganic acids and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include, e.g., hydrochloric acid, hydrobromic acid, sulfuric acid, nitric acid, phosphoric acid and the like. Salts derived from organic acids include, e.g., acetic acid, propionic acid, gluconic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid and the like. Salts derived from organic acids may be derived from anhydrous organic acids or hydrates thereof. Likewise, pharmaceutically acceptable base addition salts can be prepared from inorganic and organic bases. Salts derived from inorganic bases include, by way of example only, sodium, potassium, lithium, aluminum, ammonium, calcium and magnesium salts. Salts derived from organic bases include, but are not limited to, salts of primary, secondary and tertiary amines, such as alkyl amines (i.e., NH₂(alkyl)), dialkyl amines (i.e., HN(alkyl)₂), trialkyl amines (i.e., N(alkyl)₃), substituted alkyl amines (i.e., NH₂(substituted alkyl)), di(substituted alkyl) amines (i.e., HN(substituted alkyl)₂), tri(substituted alkyl) amines (i.e., N(substituted alkyl)₃), alkenyl amines (i.e., NH₂(alkenyl)), dialkenyl amines (i.e., HN(alkenyl)₂), dialkenyl amines (i.e., N(alkenyl)₃), substituted alkenyl amines (i.e., NH₂(substituted alkenyl)), di(substituted alkenyl) amines (i.e., HN(substituted alkenyl)₂), tri(substituted alkenyl) amines (i.e., N(substituted alkenyl)₃, mono-, di- or tri-cycloalkyl amines (i.e., NH₂(cycloalkyl), HN(cycloalkyl)₂, N(cycloalkyl)₃), mono-, di- or tri-arylamines (i.e., NH₂(aryl), HN(aryl)₂, N(aryl)₃) or mixed amines, etc. Specific examples of suitable amines include, by way of example only, isopropylamine, trimethyl amine, diethyl amine, tri(isopropyl) amine, tri(n-propyl) amine, ethanolamine, 2-dimethylaminoethanol, piperazine, piperidine, morpholine, N-ethylpiperidine, and the like.

Some of the compounds exist as tautomers. Tautomers are in equilibrium with one another. For example, amide containing compounds may exist in equilibrium with imidic acid tautomers. Regardless of which tautomer is shown and regardless of the nature of the equilibrium among tautomers, the compounds are understood by one of ordinary skill in the art to comprise tautomers. Thus, the amide containing compounds are understood to include their imidic acid tautomers. Likewise, the imidic acid containing compounds are understood to include their amide tautomers.

A “stereoisomer” refers to a compound made up of the same atoms bonded by the same bonds but having different three-dimensional structures, which are not interchangeable. The present disclosure contemplates various stereoisomers and mixtures thereof and includes “

Stereoisomers include enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). Stereoisomers also include geometric isomers when the compounds described herein contain olefinic double bonds or other centers of geometric asymmetry. Unless specified otherwise, it is intended that such compounds include both E and Z geometric isomers.

“Enantiomers” are two stereoisomers whose molecules are non-superimposable mirror images of one another. “Diastereomers” are stereoisomers that have at least two asymmetric atoms, but which are not mirror-images of each other.

Relative centers of the compounds as depicted herein are indicated graphically using the “thick bond” style and absolute stereochemistry is depicted using wedge bonds.

When the stereochemistry of a disclosed compound is named or depicted by structure, the named or depicted stereoisomer is at least 60%, 70%, 80%, 90%, 99% or 99.9% by weight pure relative to the other stereoisomers. When a single enantiomer is named or depicted by structure, the depicted or named enantiomer is at least 60%, 70%, 80%, 90%, 99% or 99.9% by weight optically pure. Percent optical purity by weight is the ratio of the weight of the enantiomer over the weight of the enantiomer plus the weight of its optical isomer.

When the geometry of a disclosed compound is named or depicted by structure, the named or depicted geometrical isomer is at least 60%, 70%, 80%, 90%, 99% or 99.9% by weight pure relative to the other geometrical isomers.

In certain embodiments, where one or more stereocenters are present, a compound disclosed herein may be provided as a racemic mixture. In certain embodiments, where one or more stereocenters are present, a compound disclosed herein may be provided as a single enantiomer. For example, a compound may be provided in a composition having greater than about 30% ee, about 40% ee, about 50% ee, about 60% ee, about 70% ee, about 80% ee, about 90% ee, about 95% ee, about 97% ee, about 98% ee, about 99% ee, or greater. In certain such embodiments, compounds may be provided in a diastereomerically enriched composition. For example, a diastereomerically enriched composition comprising a compound disclosed herein may have greater than about 30% de, about 40% de, about 50% de, about 60% de, about 70% de, about 80% de, about 90% de, about 95% de, about 97% de, about 98% de, about 99% de, or greater.

In certain embodiments, the therapeutic preparation may be enriched to provide predominantly one enantiomer of a compound (e.g., of Formula (I)). An enantiomerically enriched mixture may comprise, for example, at least about 60 mol percent of one enantiomer, or more preferably at least about 75, about 90, about 95, or even about 99 mol percent. In certain embodiments, the compound enriched in one enantiomer is substantially free of the other enantiomer, wherein substantially free means that the substance in question makes up less than about 10%, or less than about 5%, or less than about 4%, or less than about 3%, or less than about 2%, or less than about 1% as compared to the amount of the other enantiomer, e.g., in the composition or compound mixture. For example, if a composition or compound mixture contains about 98 grams of a first enantiomer and about 2 grams of a second enantiomer, it would be said to contain about 98 mol percent of the first enantiomer and only about 2% of the second enantiomer.

In certain embodiments, the therapeutic preparation may be enriched to provide predominantly one diastereomer of a compound (e.g., of Formula (I)). A diastereomerically enriched mixture may comprise, for example, at least about 60 mol percent of one diastereomer, or more preferably at least about 75, about 90, about 95, or even about 99 mol percent.

The term “prodrug” is intended to encompass compounds which, under physiologic conditions, are converted into the therapeutically active agents of the present disclosure (e.g., a compound of Formula (I)). A common method for making a prodrug is to include one or more selected moieties which are hydrolyzed under physiologic conditions to reveal the desired molecule. In other embodiments, the prodrug is converted by an enzymatic activity of the subject. For example, esters or carbonates (e.g., esters or carbonates of alcohols or carboxylic acids) are preferred prodrugs of the present disclosure. In certain embodiments, some or all of the compounds of Formula (I) in a formulation represented above can be replaced with the corresponding suitable prodrug, e.g., wherein a hydroxyl in the parent compound is presented as an ester or a carbonate or carboxylic acidA “therapeutically effective amount”, as used herein refers to an amount that is sufficient to achieve a desired therapeutic effect. For example, a therapeutically effective amount can refer to an amount that is sufficient to improve at least one sign or symptom of diseases or conditions disclosed herein.

Abbreviations:

| | |
|---|---|
| DCM | dichloromethane |
| DIPEA | diisopropylethylamine |
| DMA | dimethylacetamide |
| DMAP | dimethylaminopyridine |
| DMF | dimethylformamide |
| DMSO | dimethyl sulfoxide |
| EDCI | 1-ethyl-3-(3-dimethylaminopropyl)carbodiimide |
| Equiv or eq | equivalent |
| ESI | electrospray ionization |
| EtOAc | ethyl acetate |
| EtOH | ethanol |
| EtONa | sodium ethoxide |
| HOAc or AcOH | acetic acid |
| HOBt | 1-hydroxybenzotriazole |
| HPLC | high performance liquid chromatography |
| HRMS | high-resolution mass spectrometry |
| LC | liquid chromatography |
| LCMS | liquid chromatography-mass spectrometry |
| mCPBA | meta-chloroperoxybenzoic acid |
| MeOH | methanol |
| NMM | N-methylmorpholine |
| NMP | N-methyl-2-pyrrolidone |
| OXONE ® | Potassium peroxymonosulfate |
| PPSE | Trimethylsilyl polyphosphate |
| rt | room temperature |
| TEA | triethylamine |
| THF | tetrahydrofuran |
| TLC | thin-layer chromatography |
| TsOH | p-toluenesulfonic acid |

B. Compounds

Small molecules have been identified that bind to and activate PLXDC1 and PLXDC2 proteins, leading to effective killing of the endothelial cells in pathogenic blood vessels that express these proteins. In certain embodiments, provided is a compound of Formula (I):

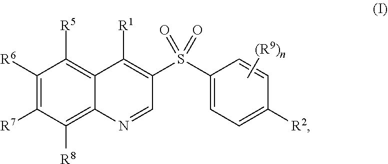

or a pharmaceutically acceptable salt, isotopically enriched analog, stereoisomer, mixture of stereoisomers, or tautomer thereof;
wherein
n is 0, 1, 2, 3 or 4;
R¹ is selected from optionally substituted amino, optionally substituted aryl, optionally substituted cycloalkyl, optionally substituted heterocyclyl, and optionally substituted heteroaryl;
R² is selected from H, halo, alkyl, alkenyl, alkynyl, —OH, alkoxy, —CN, —NO₂, alkylthio, sulfoxido, sulfonyl, and amino;
R⁵, R⁷ and R⁸ are each independently selected from H, halo, alkyl, alkenyl, alkynyl, hydroxy, alkoxy, alkenoxy, alkynoxy, alkylthio, sulfoxido, sulfonyl, carboxy, ester, —CN, —NO₂, amino, and amido;
R⁶ is selected from H, halo, alkyl, hydroxy, alkoxy, alkylthio, sulfoxido, sulfonyl, carboxy, ester, —CN, —NO₂, amino, amido, sulfinamido, sulfonamido, optionally substituted heterocyclyl, optionally substituted heteroaryl, poly(ethylene glycol), and methoxypoly(ethylene glycol), or
R⁶ and IV together with atoms to which they are attached form an optionally substituted cycloalkyl, optionally substituted heterocyclyl, optionally substituted aryl, or optionally substituted heteroaryl; and
each R⁹ is independently selected from halo, alkyl, —OH, alkoxy, —CN, and amino.

In certain embodiments, when R² is C₁₋₆ alkyl, R⁶ is not C₁₋₆ alkyl or C₁₋₆ alkoxy. In other embodiments, when R² is C₁₋₃ alkyl, R⁶ is not C₁₋₃ alkyl or C₁₋₃ alkoxy. In certain embodiments, when R² is ethyl, then R⁶ is not methyl or methoxy.

In certain embodiments, provided is a compound of Formula (I):

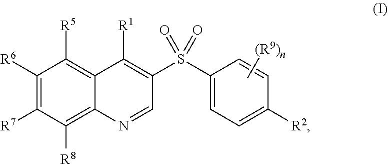

or a pharmaceutically acceptable salt, isotopically enriched analog, stereoisomer, mixture of stereoisomers, or tautomer thereof;
wherein
n is 0, 1, 2, 3 or 4;
R¹ is selected from optionally substituted amino, optionally substituted aryl, optionally substituted cycloalkyl, optionally substituted heterocyclyl, and optionally substituted heteroaryl;
R² is selected from halo, alkyl, alkenyl, alkynyl, alkoxy, alkenoxy, alkynoxy, —CN, and —NO₂;
R⁵, R⁷ and R⁸ are each independently selected from H, halo, alkyl, alkenyl, hydroxy, and alkoxy;
R⁶ is selected from halo, alkyl, alkenyl, alkynyl hydroxy, alkoxy, alkylthio, sulfoxide, sulfonyl, carboxy, ester, —NO₂—CN, amino, and amido; and
each R⁹ is independently selected from halo, hydroxy, and alkoxy.

In certain embodiments, provided is a compound of Formula (I):

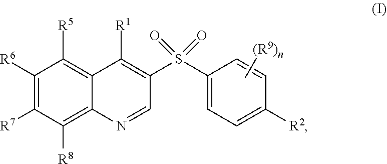

or a pharmaceutically acceptable salt, isotopically enriched analog, stereoisomer, mixture of stereoisomers, or tautomer thereof;
wherein
n is 0 or 1;
R¹ is selected from amino, optionally substituted heterocyclyl, and optionally substituted heteroaryl;
R² is selected from alkyl, alkoxy, —CN, and —NO₂;
R⁵, R⁷ and R⁸ are each independently selected from H and alkoxy;
R⁶ is selected from halo, alkyl, alkoxy, sulfoxido, sulfonyl, carboxy, ester, —NO₂ and amido; and
each R⁹ is independently halo or alkoxy.

In certain embodiments, the compound of Formula (I) described above has at least one of the following:
1) R² is selected from C₂₋₃₀alkyl, —OH, C₁₋₄₀ alkoxy, C₁₋₄₀ alkenoxy, C₁₋₄₀ alkynoxy, —NO₂, alkylthio, sulfoxido, sulfonyl, and amino, where
a) when R² is ethyl, then R⁶ is not methyl or methoxy, and/or
b) when R² is methoxy, then R⁶ is not halo, C₁₋₂ alkyl or C₁₋₂ alkoxy;
2) R¹ is optionally substituted heteroaryl, optionally substituted bridged heterocyclyl, optionally substituted fused heterocyclyl, or optionally substituted cycloheptyl;
3) R¹ is heterocyclyl optionally substituted with one halo, amino, hydroxy, alkoxy, —CN, —NO₂, alkyl, carboxy, alkylthio, sulfoxido, sulfonyl, sulfinamido, sulfonamido, cycloalkyl, heterocyclyl, heteroaryl, poly(ethylene glycol), or methoxypoly(ethylene glycol),
where if the substituent is alkyl, the alkyl is further substituted with one substituent selected from halo, amino, alkoxy, —CN, —NO₂, carboxy, ester, alkylthio, sulfoxido, sulfonyl, sulfinamido, sulfonamido, cycloalkyl, heterocyclyl, poly(ethylene glycol), methoxypoly(ethylene glycol), pyrrolidinyl and piperidinyl; or the alkyl is substituted with at least one —OR³¹, wherein R³¹ is poly(ethylene glycol) or methoxypoly(ethylene glycol);
4) R¹ is amino substituted with at least one substituent selected from alkyl, cycloalkyl, heterocyclyl, heteroaryl, poly(ethylene glycol) or methoxypoly(ethylene glycol) and amino,
where the alkyl is substituted with at least one substituent selected from halo, amino, hydroxy, alkoxy, —CN, —NO₂, amido, carboxy, ester, alkylthio, sulfoxido, sulfonyl, sulfinamido, sulfonamido, cycloalkyl, heterocyclyl, and heteroaryl; or
5) R⁶ is alkyl substituted with at least one substituent selected from halo, amino, hydroxy, alkoxy, cycloalkoxy, heterocycloalkoxy, aryloxy, heteroaryloxy, poly(ethylene glycol)-oxy, methoxypoly(ethylene glycol)-oxy, —CN, —NO₂, oxo, amido, carboxy, ester, alkylthio, sulfoxido, sulfonyl, sulfinamido, sulfonamido, heterocyclyl, cycloalkyl, aryl, heteroaryl, poly(ethylene glycol) and methoxypoly(ethylene glycol).

In certain embodiments, R¹ is heterocyclyl substituted with at least one substituent selected from oxo, —OH, —OR²⁸, —N(R²⁸)₂, —C(O)OR²⁸, alkyl, aryl, and heterocyclyl, wherein the alkyl is substituted with at least one substituent selected from —OH, —N(R³¹)₂, —S(O)₀₋₂NR³¹R³¹, —C(O)N(R³¹)₂, heterocyclyl, cycloalkyl, pyrrolidinyl and piperidinyl; or the alkyl is substituted with at least one —OR′, wherein R^{31a} is poly(ethylene glycol) or methoxypoly(ethylene glycol); and each R²⁸ and R³¹ are independently H or alkyl.

In certain embodiments, R¹ is —NR³R⁴, and R³ is H or alkyl unsubstituted or substituted with at least one substituent selected from —OH, —N(R²⁸)₂, aryl, and heteroaryl, and R⁴ and R²⁸ are each independently H or alkyl.

In certain embodiments, a compound of Formula (I) is a compound of Formula (II) and/or (Formula III) and/or Formula (IV) and/or (Formula V) and/or Formula (VI) and/or Formula (VII) and/or Formula (VIII) and/or Formula (IX) and/or Formula (X) and/or Formula (XI) and/or Formula (XII), as described herein, or any combination thereof.

In certain embodiments, provided is a compound of Formula (II):

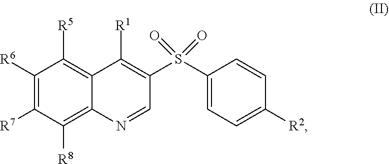

or a pharmaceutically acceptable salt, isotopically enriched analog, stereoisomer, mixture of stereoisomers, or tautomer thereof, wherein each of R¹, R², R⁵, R⁶, R⁷, and R⁸ is as defined herein.

In certain embodiments, provided is a compound of Formula (III):

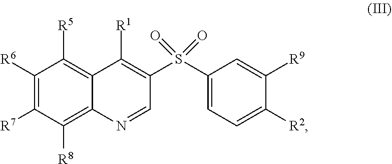

or a pharmaceutically acceptable salt, isotopically enriched analog, stereoisomer, mixture of stereoisomers, or tautomer thereof, wherein each of R¹, R², R⁵, R⁶, R⁷, R⁸ and R⁹ is as defined herein.

In certain embodiments, provided is a compound of Formula (IV):

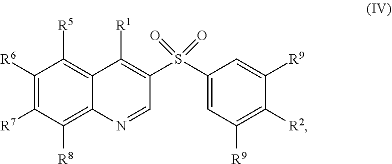

or a pharmaceutically acceptable salt, isotopically enriched analog, stereoisomer, mixture of stereoisomers, or tautomer thereof, wherein each of R¹, R², R⁵, R⁶, R⁷, R⁸ and R⁹ is as defined herein.

In certain embodiments, R¹ is heteroaryl or heterocyclyl, each optionally substituted with a second heterocyclyl, wherein the second heterocyclyl is unsubstituted or substituted with one or more substituents, e.g., selected from —OH, —C(O)Oalkyl, —C(O)NHalkyl, alkyl, aryl, and heterocyclyl;
wherein the alkyl and heterocyclyl are each unsubstituted or substituted with one or more substituents selected from —OH, alkyl and aryl.

In certain embodiments, provided is a compound of Formula (V):

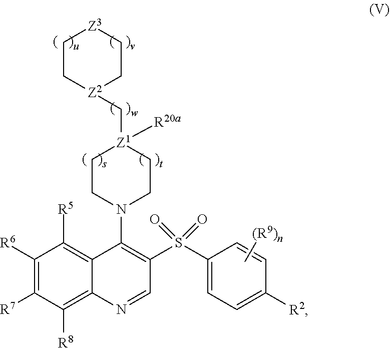

or a pharmaceutically acceptable salt, isotopically enriched analog, stereoisomer, mixture of stereoisomers, or tautomer thereof,
wherein
each of s, t, u, and v is independently 0, 1, 2, or 3, provided that the sum of s and t is 1, 2, 3 or 4, and the sum of u and v is 1, 2, 3 or 4;
w is 0, 1, 2, or 3;
Z¹ is C or N,
when Z¹ is C, R^{20a} is H, halo, oxo, —NH₂, —OH, —CN, —NO₂, —NHR²⁸, —N(R²⁸)₂, —C(O)R²⁸, —C(O)OR²⁸, —C(O)OH, —OC(O)R²⁸, —S(O)₀₋₂R²⁸, —NHS(O)₀₋₂R²⁸, —S(O)₀₋₂NHR²⁸, —NHS(O)₀₋₂NHR²⁸, —C(O)NH₂, —C(O)NHR²⁸, —C(O)N(R²⁸)₂, —NHC(O)R²⁸, —OC(O)NHR²⁸, —NHC(O)OR²⁸, —OC(O)N(R²⁸)₂, —NR³²C(O)NH₂, —NR³²C(O)NHR²⁸, —NR³²C(O)N(R²⁸)₂, poly(ethylene glycol), methoxypoly(ethylene glycol), C₁₋₃₀ alkyl optionally substituted with OH or —C(O)OH, or C₁₋₃₀ heteroalkyl optionally substituted with OH or —C(O)OH, wherein R³² is H or C₁₋₄ alkyl, and R²⁸ is C₁₋₄ alkyl;
when Z¹ is N, R^{20a} is absent;
Z² is C or N;
Z³ is CH₂, CHR²⁵, CR²⁵R²⁵, or NR²⁵, O, or S(O)₀₋₂ and R²⁵ is selected from H and alkyl; and
each of n, R², R⁵, R⁶, IV, R⁸, and R⁹ is as defined herein.

In certain embodiments, provided is a compound of Formula (V):

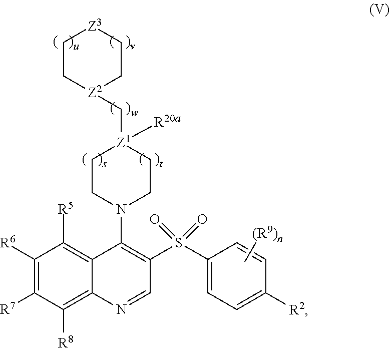

or a pharmaceutically acceptable salt, isotopically enriched analog, stereoisomer, mixture of stereoisomers, or tautomer thereof,
wherein
each of s, t, u, and v is independently 0, 1, 2, or 3, provided that the sum of s and t is 1, 2, 3 or 4, and the sum of u and v is 1, 2, 3 or 4;
w is 0, 1, 2, or 3;
Z¹ is C or N,
when Z¹ is C, R^{20a} is H, halo, oxo, —NH₂, —OH, —CN, —NO₂, —NHR²⁸, —N(R²⁸)₂, —C(O)R²⁸, —C(O)R²⁸, —C(O)OH, —OC(O)R²⁸, —S(O)₀₋₂R²⁸, —NHS(O)₀₋₂R²⁸, —S(O)₀₋₂NHR²⁸, —NHS(O)₀₋₂NHR²⁸, —C(O)NH₂, —C(O)NHR²⁸, —C(O)N(R²⁸)₂, —NHC(O)R²⁸, —OC(O)NHR²⁸, —NHC(O)OR²⁸, —OC(O)N(R²⁸)₂, —NR³²C(O)NH₂, —NR³²C(O)NHR²⁸, —NR³²C(O)N(R²⁸)₂, poly(ethylene glycol), methoxypoly(ethylene glycol), C₁₋₃₀ alkyl optionally substituted with OH or —C(O)OH, or C₁₋₃₀ heteroalkyl optionally substituted with OH or —C(O)OH, wherein R³² is H or C₁₋₄ alkyl, and R²⁸ is C₁₋₄ alkyl;
when Z¹ is N, R^{20a} is absent;
Z² is C or N;
Z³ is CH₂, CHR²⁵, CR²⁵R²⁵, or NR²⁵, O, or S(O)₀₋₂ and R²⁵ is selected from H and alkyl;
n is 0, 1, 2, 3 or 4;
R² is selected from H, halo, alkyl, alkenyl, alkynyl, —OH, alkoxy, —CN, —NO₂, alkylthio, sulfoxido, sulfonyl, and amino;
R⁵, R⁷ and R⁸ are each independently selected from H, halo, alkyl, alkenyl, alkynyl, hydroxy, alkoxy, alkylthio, sulfoxido, sulfonyl, carboxy, ester, —CN, —NO₂, amino, and amido;
R⁶ is selected from H, halo, alkyl, hydroxy, alkoxy, alkylthio, sulfoxido, sulfonyl, carboxy, ester, —CN, —NO₂, amino, amido, sulfinamido, sulfonamido, heterocyclyl, heteroaryl, poly(ethylene glycol), and methoxypoly(ethylene glycol), or
R⁶ and R⁷ together with atoms to which they are attached form a cycloalkyl, heterocyclyl, aryl, or heteroaryl; and
each R⁹ is independently selected from halo, alkyl, —OH, alkoxy, —CN, and amino.

In certain embodiments, provided is a compound of a formula (VI):

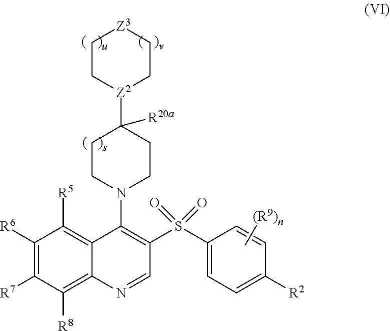

or a pharmaceutically acceptable salt, isotopically enriched analog, stereoisomer, mixture of stereoisomers, or tautomer thereof, wherein each of n, s, u, v, R², R⁵, R⁶, R⁷, R⁸, R⁹, R^{20a}, Z², and Z³ is as defined herein.

In certain embodiments, provided is a compound of a formula:

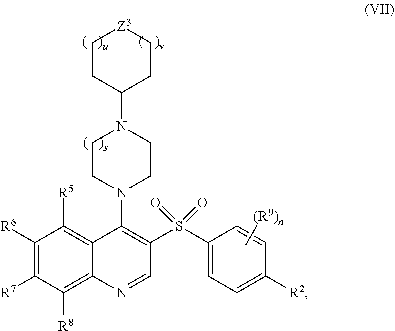

or a pharmaceutically acceptable salt, isotopically enriched analog, stereoisomer, mixture of stereoisomers, or tautomer thereof, wherein each of n, s, u, v, R², R⁵, R⁶, R⁷, R⁸, R⁹, and Z³ is as defined herein.

In certain embodiments, provided is a compound of a formula (VIII):

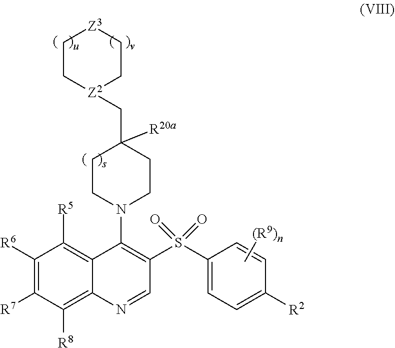

or a pharmaceutically acceptable salt, isotopically enriched analog, stereoisomer, mixture of stereoisomers, or tautomer thereof, wherein each of n, s, u, v, R², R⁵, R⁶, R⁷, R⁸, R⁹, R^{20a}, Z², and Z³ is as defined herein.

In certain embodiments, provided is a compound of a formula (IX):

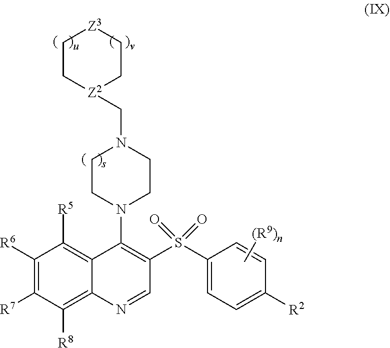

or a pharmaceutically acceptable salt, isotopically enriched analog, stereoisomer, mixture of stereoisomers, or tautomer thereof, wherein each of n, s, u, v, R², R⁵, R⁶, R⁷, R⁸, R⁹, Z², and Z³ is as defined herein.

In certain embodiments, provided is a compound of a formula (X):

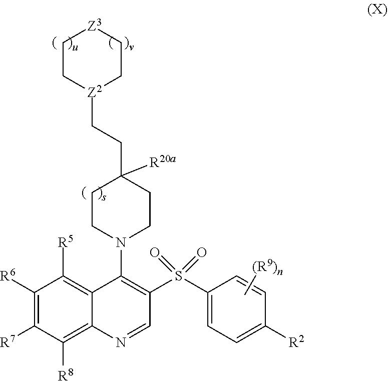

or a pharmaceutically acceptable salt, isotopically enriched analog, stereoisomer, mixture of stereoisomers, or tautomer thereof, wherein each of n, s, u, v, R², R⁵, R⁶, R⁷, R⁸, R⁹, R^{20a}, ZZ, and Z³ is as defined herein.

In certain embodiments, provided is a compound of a formula (XI):

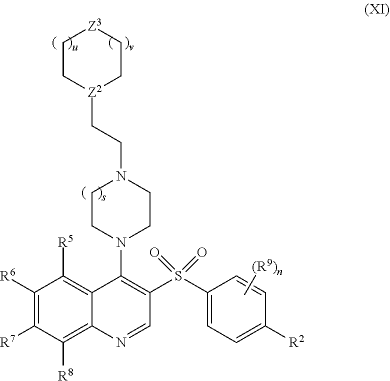

or a pharmaceutically acceptable salt, isotopically enriched analog, stereoisomer, mixture of stereoisomers, or tautomer thereof, wherein each of n, s, u, v, R², R⁵, R⁶, R⁷, R⁸, R⁹, Z², and Z³ is as defined herein.

In certain embodiments, provided is a compound of Formula (I):

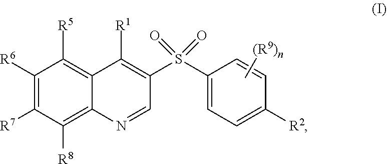

or a pharmaceutically acceptable salt, isotopically enriched analog, stereoisomer, mixture of stereoisomers, or tautomer thereof;
   wherein
R² is —OC₄₋₃₀ alkyl; and
each of n, IV, R⁵, R⁶, R⁷, R⁸, and R⁹ is as defined herein.

In certain embodiments, provided is a compound of Formula (I):

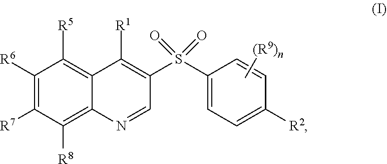

or a pharmaceutically acceptable salt, isotopically enriched analog, stereoisomer, mixture of stereoisomers, or tautomer thereof;
   wherein
R⁶ is —NO₂, ester, amido, alkylthio, sulfoxido, or sulfonyl; and
each of n, R¹, R⁵, R⁵, R⁷, R⁸, R⁹ is as defined herein.

In certain embodiments, R⁹ is —OH or —O—C₁₋₁₀ alkyl. In certain embodiments, each R⁹ is independently halo. In certain embodiments, n is 1 or 2 and each R⁹ is independently halo. In certain embodiments, n is 1 or 2 and each R⁹ is fluoro.

In certain embodiments, provided is a compound of Formula (I):

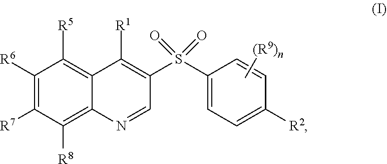

or a pharmaceutically acceptable salt, isotopically enriched analog, stereoisomer, mixture of stereoisomers, or tautomer thereof;
wherein
R¹ is selected from —NR³R⁴, heterocyclyl optionally substituted with one to five R²⁰, and heteroaryl optionally substituted with one to five R²¹;
R² is selected from H, halo, C₁₋₃₀ alkyl optionally substituted with one to five R¹⁹, C₁₋₃₀ heteroalkyl optionally substituted with one to five R¹⁹, —OH, —OR¹⁸, —CN, —NO₂, —NH₂, —S(O)₀₋₂R¹⁸, and —NR¹¹R¹⁸;
R³ is selected from H, C₁₋₆ alkyl optionally substituted with one to five R²⁰, C₃₋₁₀ cycloalkyl optionally substituted with one to five R²⁰, heterocyclyl optionally substituted with one to five R²⁰, aryl optionally substituted with one to five R²¹, and heteroaryl optionally substituted with one to five R²¹,
R⁴ is selected from H, C₁₋₃₀ alkyl optionally substituted with one to five R²⁰, C₁₋₃₀ heteroalkyl optionally substituted with one to five R²⁰, C₃₋₁₀ cycloalkyl optionally substituted with one to five R²⁰, heterocyclyl optionally substituted with one to five R²⁰, aryl optionally substituted with one to five R²¹, heteroaryl optionally substituted with one to five R²¹, poly(ethylene glycol), methoxypoly(ethylene glycol), and —NR¹³R¹⁴;
R⁵, R⁷ and R⁸ are each independently selected from H, halo, C₁₋₃₀ alkyl optionally substituted with one to five R²⁹, C₁₋₃₀ heteroalkyl optionally substituted with one to five R²⁹, —OR¹⁵, —S(O)₀₋₂₈R¹⁶, —C(O)OR¹⁵, —OC(O)R¹⁶, —CN, —NO₂, —NR¹⁵R¹⁵, and —C(O)NR¹⁵R¹⁵;
R⁶ is selected from H, halo, C₁₋₃₀ alkyl optionally substituted with one to five R²⁹, C₁₋₃₀ heteroalkyl optionally substituted with one to five R²⁹, —OR¹⁷, —S(O)₀₋₂R¹⁶, —C(O)OR¹⁵, —OC(O)R¹⁶, —CN, —NO₂, —NR¹⁵R¹⁵, —C(O)NR¹⁵R¹⁵, —NR¹⁵C(O)R¹⁶, S(O)₀₋₂NR¹⁵R¹⁵, —NR¹⁵S(O)₀₋₂R¹⁶, heterocyclyl optionally substituted with one to five R²⁹, heteroaryl optionally substituted with one to five R³⁰, poly(ethylene glycol), and methoxypoly(ethylene glycol), or
R⁶ and R⁷ together with atoms to which they are attached form C₅₋₁₀ cycloalkyl optionally substituted with one to five R²⁹, heterocyclyl optionally substituted with one to five R²⁹, aryl optionally substituted with one to five R³⁰, or heteroaryl optionally substituted with one to five R³⁰;
R⁹ is independently selected from halo, C₁₋₈ alkyl optionally substituted with one to five R²², —OH, —OR¹⁰, —CN, and —NR¹¹R¹²;
n is 0, 1, 2, 3 or 4;
R¹⁰ is independently selected from C₁₋₁₀ alkyl optionally substituted with one to five R²², and C₃₋₁₀ cycloalkyl optionally substituted with one to five R²²;
R¹¹ and R¹² are each independently H or C₁₋₆ alkyl optionally substituted with one to five R²²;
R¹³ is selected from H, C₁₋₆ alkyl optionally substituted with one to five R²⁰, C₃₋₁₀ cycloalkyl optionally substituted with one to five R²⁰, heterocyclyl optionally substituted with one to five R²⁰, aryl optionally substituted with one to five R²¹, and heteroaryl optionally substituted with one to five R²¹;
R¹⁴ is selected from H, C₁₋₃₀ alkyl optionally substituted with one to five R²⁰, C₁₋₃₀ heteroalkyl optionally substituted with one to five R²⁰, C₃₋₁₀ cycloalkyl optionally substituted with one to five R²⁰, heterocyclyl optionally substituted with one to five R²⁰, aryl optionally substituted with one to five R²¹, heteroaryl optionally substituted with one to five R²¹, poly(ethylene glycol), and methoxypoly(ethylene glycol);
each R¹⁵ is independently selected from H, C₁₋₃₀ alkyl optionally substituted with one to five R²³, C₁₋₃₀ heteroalkyl optionally substituted with one to five R²³, C₃₋₁₀ cycloalkyl optionally substituted with one to five R²³, heterocyclyl optionally substituted with one to five R²³, aryl optionally substituted with one to five R²⁴, heteroaryl optionally substituted with one to five R²⁴, poly(ethylene glycol), and methoxypoly(ethylene glycol);
R¹⁶ is selected from C₁₋₃₀ alkyl optionally substituted with one to five R²³, C₁ heteroalkyl optionally substituted with one to five R²³, C₃₋₁₀ cycloalkyl optionally substituted with one to five R²³, heterocyclyl optionally substituted with one to five R²³, aryl optionally substituted with one to five R²⁴, heteroaryl optionally substituted with one to five R²⁴, poly(ethylene glycol), and methoxypoly(ethylene glycol);
R¹⁷ is selected from H, C₁₋₃₀ alkyl optionally substituted with one to five R²⁷, C₁₋₃₀ heteroalkyl optionally substituted with one to five R²⁷, C₃₋₁₀ cycloalkyl optionally substituted with one to five R²⁷, heterocyclyl optionally substituted with one to five R²⁷, aryl optionally substituted with one to five R²⁴, heteroaryl optionally substituted with one to five R²⁴, poly(ethylene glycol), and methoxypoly(ethylene glycol);
R¹⁸ is selected from C₁₋₃₀ alkyl optionally substituted with one to five R¹⁹ and C₁₋₃₀ heteroalkyl optionally substituted with one to five R¹⁹;
each R²⁰ is independently selected from halo, oxo, —NH₂, —OH, —CN, —NO₂, —OR²⁸, —NHR²⁸, —N(R²⁸)₂, —C(O)R²⁸, —C(O)OR²⁸, —C(O)OH, —OC(O)R²⁸, —S(O)₀₋₂R²⁸, —NHS(O)₀₋₂R²⁸, —S(O)₀₋₂NHR²⁸, —NHS(O)₀₋₂NHR²⁸, —C(O)NH₂, —C(O)NHR²⁸, —C(O)N(R²⁸)₂, —NHC(O)R²⁸, —OC(O)NHR²⁸, —NHC(O)OR²⁸, —OC(O)N(R²⁸)₂, —NR³²C(O)NH₂, —NR³²C(O)NHR²⁸, —NR³²C(O)N(R²⁸)₂, C₁₋₃₀ alkyl optionally substituted with one to five R²⁵, C₁₋₃₀ heteroalkyl optionally substituted with one to five R²⁵, C₃₋₁₀ cycloalkyl optionally substituted with one to five R²⁵, heterocyclyl optionally substituted with one to five R²⁵, aryl optionally substituted with one to five R²⁶, heteroaryl optionally substituted with one to five R²⁶, poly(ethylene glycol), and methoxypoly(ethylene glycol);
each R²¹ is independently selected from halo, —NH₂, —OH, —CN, —NO₂, —OR²⁸, —NHR²⁸, —N(R²⁸)₂, —C(O)R²⁸, —C(O)OR²⁸, —C(O)OH, —OC(O)R²⁸, —S(O)₀₋₂R²⁸, —NHS(O)₀₋₂R²⁸, —S(O)₀₋₂NHR²⁸, —NHS(O)₀₋₂NHR²⁸, —C(O)NHR²⁸, —NHC(O)R²⁸, —C(O)N(R²⁸)₂, —OC(O)NHR²⁸, —NHC(O)OR²⁸, —OC(O)N(R²⁸)₂, —NR³²C(O)NH₂, —NR³²C(O)NHR²⁸, —NR³²C(O)N(R²⁸)₂, C₁₋₆ alkyl optionally substituted with one to five R²⁵, C₁₋₃₀ heteroalkyl optionally substituted with one to five R²⁵, C₃₋₁₀ cycloalkyl optionally substituted with one to five R²⁵, heterocyclyl optionally substituted with one to five R²⁵, aryl optionally substituted with one to five R²⁶, and heteroaryl optionally substituted with one to five R²⁶, poly(ethylene glycol), and methoxypoly(ethylene glycol);
each R¹⁹ or R²² is independently selected from halo, —NH₂, —OH, —CN, —NO₂, oxo, C₁₋₄ alkyl, C₁₋₄ haloalkyl, C₁₋₄ hydroxyalkyl, C₁₋₄ alkoxy, —NH—C₁₋₄ alkyl, —N(C₁₋₄ alkyl)₂, —C(O)—C₁₋₄ alkyl, —C(O)O—C₁₋₄ alkyl, —C(O)OH, —S(O)₀₋₂—C₁₋₄ alkyl, —NHS(O)₀₋₂—C₁₋₄ alkyl, —S(O)₀₋₂NH—C₁₋₄ alkyl, —NHS(O)₀₋₂NH—C₁₋₄ alkyl, —C(O)NH—C₁₋₄ alkyl, —NHC(O)—C₁₋₄ alkyl, —C(O)N(C₁₋₄ alkyl)₂, —OC(O)NH—C₁₋₄ alkyl, —NHC(O)O—C₁₋₄ alkyl, —OC(O)N(C₁₋₄ alkyl)₂, —NH—C₁₋₄ haloalkyl, —N(C₁₋₄ haloalkyl)₂, —C(O)—C₁₋₄ haloalkyl, —S(O)₀₋₂—C₁₋₄ haloalkyl, —NHS(O)₀₋₂—C₁₋₄ haloalkyl, —S(O)₀₋₂NH—C₁₋₄ haloalkyl, —NHS(O)₀₋₂NH—C₁₋₄ haloalkyl, —C(O)NH—C₁₋₄ haloalkyl, —NHC(O)—C₁₋₄ haloalkyl, —C(O)N(C₁₋₄ haloalkyl)₂, —OC(O)NH—C₁₋₄ haloalkyl, —NHC(O)O—C₁₋₄ haloalkyl, —OC(O)N(C₁₋₄ haloalkyl)₂, and C₃₋₁₀ cycloalkyl;
each R²⁵ is independently selected from halo, —NH₂, —OH, —CN, —NO₂, oxo, —OR³¹, —NHR³¹, —N(R³¹)₂, —C(O)R³¹, —C(O)OR³¹, —C(O)OH, —S(O)₀₋₂R³¹, —NR³²S(O)₀₋₂R³¹, —S(O)₀₋₂NR³²R³¹, —NR³²S(O)₀₋₂NR³²R³¹, —C(O)NH₂, —C(O)NHR³¹, —NR³²C(O)R³¹, —C(O)N(R³¹)₂, —OC(O)NHR³¹, —NR³²C(O)OR³¹, —OC(O)N(R³¹)₂, —NR³²C(O)NH₂, —NR³²C(O)NHR³¹, —NR³²C(O)N(R³¹)₂, C₁₋₃₀ alkyl optionally substituted with one to five R³³, C₁₋₃₀ heteroalkyl optionally substituted with one to five R³³, C₃₋₁₀ cycloalkyl optionally substituted with one to five R³³, heterocyclyl optionally substituted with one to five R³³, aryl optionally substituted with one to five R³⁴, heteroaryl optionally substituted with one to five R³⁴, poly(ethylene glycol), and methoxypoly(ethylene glycol);
each R²⁶ is independently selected from halo, —NH₂, —OH, —CN, —NO₂, —NHR³¹, —N(R³¹)₂, —C(O)R³¹, —C(O)OR³¹, —C(O)OH, —S(O)₀₋₂R³¹, —NR³²S(O)₀₋₂R³¹, —S(O)₀₋₂NR³²R³¹, —NR³²S(O)₀₋₂NR³²R³¹, —C(O)NH₂, —C(O)NHR³¹, —NR³²C(O)R³¹, —C(O)N(R³¹)₂, —OC(O)NHR³¹, —NR³²C(O)OR³¹, —OC(O)N(R³¹)₂, —NR³²C(O)NH₂, —NR³²C(O)NHR³¹, —NR³²C(O)N(R³¹)₂, C₁₋₃₀ alkyl optionally substituted with one to five R³³, C₁₋₃₀ heteroalkyl optionally substituted with one to five R³³, C₃₋₁₀ cycloalkyl optionally substituted with one to five R³³, heterocyclyl optionally substituted with one to five R³³, aryl optionally substituted with one to five R³⁴, and heteroaryl optionally substituted with one to five R³⁴, poly(ethylene glycol), and methoxypoly(ethylene glycol);
each R³¹ is independently selected C₁₋₃₀ alkyl optionally substituted with one to five R³³, C₁₋₃₀ heteroalkyl optionally substituted with one to five R³³, C₃₋₁₀ cycloalkyl optionally substituted with one to five R³³, heterocyclyl optionally substituted with one to five R³³, aryl optionally substituted with one to five R³⁴, heteroaryl optionally substituted with one to five R³⁴, poly(ethylene glycol) and methoxypoly(ethylene glycol);
each R³² is independently selected H and C₁₋₄ alkyl;
each R²³, R²⁷, R²⁹ or R³³ is independently selected from halo, —NH₂, —OH, —CN, —NO₂, oxo, C₁₋₄ alkyl optionally substituted with phenyl, C₁₋₄ haloalkyl, C₁₋₄ hydroxyalkyl, C₁₋₄ alkoxy, —NH—C₁₋₄ alkyl, —N(C₁₋₄ alkyl)₂, —C(O)—C₁₋₄ alkyl, —C(O)O—C₁₋₄ alkyl, —C(O)OH, —S(O)₀₋₂—C₁₋₄ alkyl, —NHS(O)₀₋₂—C₁₋₄ alkyl, —S(O)₀₋₂ NH—C₁₋₄ alkyl, —NHS(O)₀₋₂NH—C₁₋₄ alkyl, —C(O)NH—C₁₋₄ alkyl, —NHC(O)—C₁₋₄ alkyl, —C(O)N(C₁₋₄ alkyl)₂, —OC(O)NH—C₁₋₄ alkyl, —NHC(O)O—C₁₋₄ alkyl, —OC(O)N(C₁₋₄ alkyl)₂, —NH—C₁₋₄ haloalkyl, —N(C₁₋₄ haloalkyl)₂, —C(O)—C₁₋₄ haloalkyl, —S(O)₀₋₂—C₁₋₄ haloalkyl, —NHS(O)₀₋₂—C₁₋₄ haloalkyl, —S(O)₀₋₂NH—C₁₋₄ haloalkyl, —NHS(O)₀₋₂NH—C₁₋₄ haloalkyl, —C(O)NH—C₁₋₄ haloalkyl, —NHC(O)—C₁₋₄ haloalkyl, —C(O)N(C₁₋₄ haloalkyl)₂, —OC(O)NH—C₁₋₄ haloalkyl, —NHC(O)O—C₁₋₄ haloalkyl, —OC(O)N(C₁₋₄ haloalkyl)₂, C₃₋₁₀ cycloalkyl, heterocyclyl, aryl heteroaryl, —(CH₂)₁₋₃₀—C(O)OH,
(CH₂)₀₋₄—O-poly(ethylene glycol), methoxypoly(ethylene glycol)-O—(CH₂)₀₋₄—, and sugar moiety;
   each R²⁴, R³⁰ or R³⁴ is independently selected from halo, —NH₂, —OH, —CN, —NO₂, C₁₋₄ alkyl, C₁₋₄ haloalkyl, C₁₋₄ hydroxyalkyl, C₁₋₄ alkoxy, —NH—C₁₋₄ alkyl, —N(C₁₋₄ alkyl)₂, —C(O)—C₁₋₄ alkyl, —C(O)OH, —C(O)O—C₁₋₄ alkyl, —S(O)₀₋₂—C₁₋₄ alkyl, —NHS(O)₀₋₂—C₁₋₄ alkyl, —S(O)₀₋₂NH—C₁₋₄ alkyl, —NHS(O)₀₋₂NH—C₁₋₄ alkyl, —C(O)NH—C₁₋₄ alkyl, —NHC(O)—C₁₋₄ alkyl, —C(O)N(C₁₋₄ alkyl)₂, —OC(O)NH—C₁₋₄ alkyl, —NHC(O)O—C₁₋₄ alkyl, —OC(O)N(C₁₋₄ alkyl)₂, —NH—C₁₋₄ haloalkyl, —N(C₁₋₄ haloalkyl)₂, —C(O)—C₁₋₄ haloalkyl, —S(O)₀₋₂—C₁₋₄ haloalkyl, —NHS(O)₀₋₂—C₁₋₄ haloalkyl, —S(O)₀₋₂NH—C₁₋₄ haloalkyl, —NHS(O)₀₋₂NH—C₁₋₄ haloalkyl, —C(O)NH—C₁₋₄ haloalkyl, —NHC(O)—C₁₋₄ haloalkyl, —C(O)N(C₁₋₄ haloalkyl)₂, —OC(O)NH—C₁₋₄ haloalkyl, —NHC(O)O—C₁₋₄ haloalkyl, —OC(O)N(C₁ 4 haloalkyl)₂, C₃₋₁₀ cycloalkyl, heterocyclyl, aryl, heteroaryl, —(CH₂)₁₋₃₀—C(O)OH, —(CH₂)₀₋₄—O-poly(ethylene glycol), —(CH₂)₀₋₄—O-methoxypoly(ethylene glycol) and sugar moiety; and
   each R²⁸ is independently selected from C₁₋₃₀ alkyl optionally substituted with one to five R²⁵, C₁₋₃₀ heteroalkyl optionally substituted with one to five R²⁵, C₃₋₁₀ cycloalkyl optionally substituted with one to five R²⁵, heterocyclyl optionally substituted with one to five R²⁵, aryl optionally substituted with one to five R²⁶, heteroaryl optionally substituted with one to five R²⁶, poly(ethylene glycol), and methoxypoly(ethylene glycol).

In certain embodiments, poly(ethylene glycol) has an average molecular weight of less than 20,000. In certain embodiments, poly(ethylene glycol) has an average molecular weight of less than 15,000. In certain embodiments, poly(ethylene glycol) has an average molecular weight of less than 10,000. In certain embodiments, poly(ethylene glycol) has an average molecular weight of less than 5,000. In certain embodiments, poly(ethylene glycol) has an average molecular weight of less than 2,000. In certain embodiments, poly(ethylene glycol) has an average molecular weight of about 20,000 to about 2,000. In certain embodiments, poly(ethylene glycol) has an average molecular weight of about 10,000 to about 2,000.

In certain embodiments, methoxypoly(ethylene glycol) has an average molecular weight of less than 20,000. In certain embodiments, methoxypoly(ethylene glycol) has an average molecular weight of less than 15,000. In certain embodiments, methoxypoly(ethylene glycol) has an average molecular weight of less than 15,000. In certain embodiments, methoxypoly(ethylene glycol) has an average molecular weight of less than 10,000. In certain embodiments, methoxypoly(ethylene glycol) has an average molecular weight of less than 5,000. In certain embodiments, methoxypoly(ethylene glycol) has an average molecular weight of less than 2,000. In certain embodiments, methoxypoly(ethylene glycol) has an average molecular weight of about 20,000 to about 2,000. In certain embodiments, methoxypoly(ethylene glycol) has an average molecular weight of about 10,000 to about 2,000.

In certain embodiments, provided is a compound selected from Table 3, or a pharmaceutically acceptable salt or prodrug thereof.

In certain embodiments, provided is a salt of a compound of this disclosure. In certain embodiments, the salt is salt of a compound of this disclosure formed with hydrochloric acid, hydrochloric acid, phosphoric acid, methanesulfonic acid (mesylate salt), malic acid, malonic acid, maleic acid, fumaric acid, tartaric acid, citric acid, or acetic acid. In certain embodiments, the salt is a salt of a compound of this disclosure formed with methanesulfonic acid (mesylate salt).

C. Compound agents of the disclosure

TABLE 3

| Ex. | Structure |
|---|---|
| 1 | 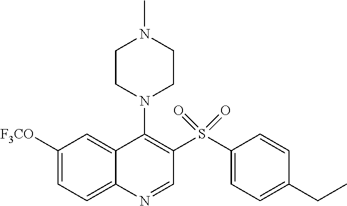 [SMILES: CCc1ccc(S(=O)(=O)c2cnc3ccc(OC(F)(F)F)cc3c2N2CCN(C)CC2)cc1] |
| 2 | 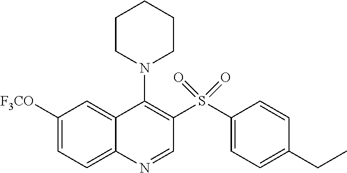 [SMILES: CCc1ccc(S(=O)(=O)c2cnc3ccc(OC(F)(F)F)cc3c2N2CCCCC2)cc1] |
| 3 | 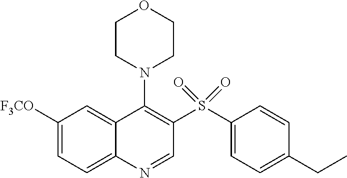 [SMILES: CCc1ccc(S(=O)(=O)c2cnc3ccc(OC(F)(F)F)cc3c2N2CCOCC2)cc1] |
| 4 | 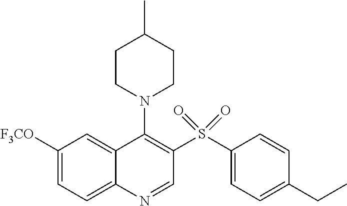 [SMILES: CCc1ccc(S(=O)(=O)c2cnc3ccc(OC(F)(F)F)cc3c2N2CCC(C)CC2)cc1] |
| 5 | 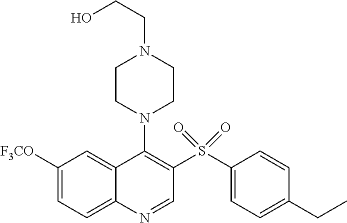 [SMILES: CCc1ccc(S(=O)(=O)c2cnc3ccc(OC(F)(F)F)cc3c2N2CCN(CCO)CC2)cc1] |
| 6 | 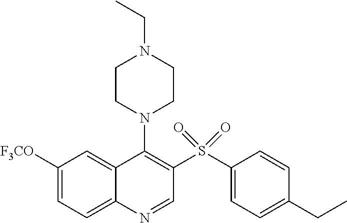 [SMILES: CCc1ccc(S(=O)(=O)c2cnc3ccc(OC(F)(F)F)cc3c2N2CCN(CC)CC2)cc1] |
| 7 | 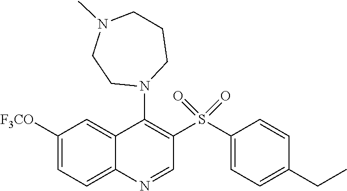 [SMILES: CCc1ccc(S(=O)(=O)c2cnc3ccc(OC(F)(F)F)cc3c2N2CCCN(C)CC2)cc1] |
| 8 | 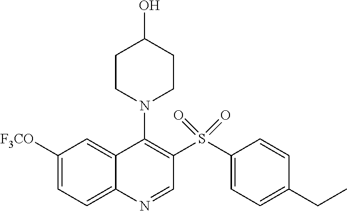 [SMILES: CCc1ccc(S(=O)(=O)c2cnc3ccc(OC(F)(F)F)cc3c2N2CCC(O)CC2)cc1] |
| 9 | 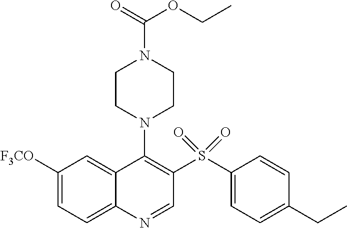 [SMILES: CCOC(=O)N1CCN(c2c(S(=O)(=O)c3ccc(CC)cc3)cnc3ccc(OC(F)(F)F)cc23)CC1] |
| 10 | 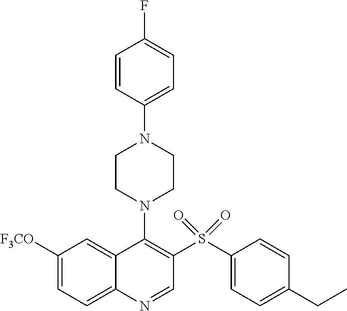 [SMILES: CCc1ccc(S(=O)(=O)c2cnc3ccc(OC(F)(F)F)cc3c2N2CCN(c3ccc(F)cc3)CC2)cc1] |
| 11 | 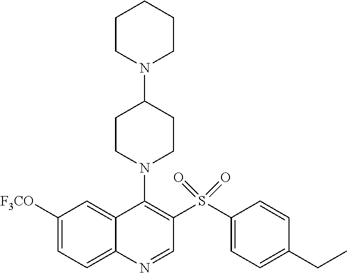 [SMILES: CCc1ccc(S(=O)(=O)c2cnc3ccc(OC(F)(F)F)cc3c2N2CCC(N3CCCCC3)CC2)cc1] |
| 12 | 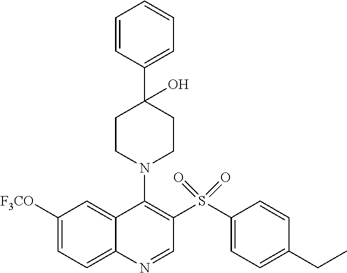 [SMILES: CCc1ccc(S(=O)(=O)c2cnc3ccc(OC(F)(F)F)cc3c2N2CCC(O)(c3ccccc3)CC2)cc1] |
| 13 | 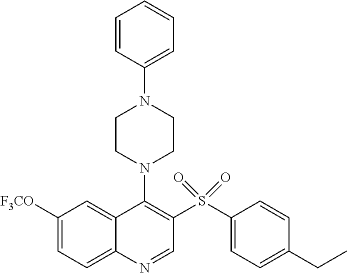 [SMILES: CCc1ccc(S(=O)(=O)c2cnc3ccc(OC(F)(F)F)cc3c2N2CCN(c3ccccc3)CC2)cc1] |
| 14 | 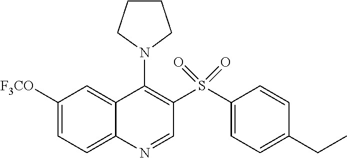 [SMILES: CCc1ccc(S(=O)(=O)c2cnc3ccc(OC(F)(F)F)cc3c2N2CCCC2)cc1] |
| 15 | 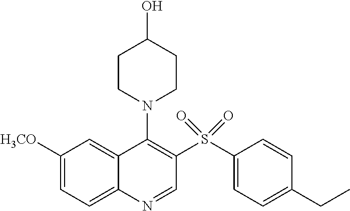 [SMILES: CCc1ccc(S(=O)(=O)c2cnc3ccc(OC)cc3c2N2CCC(O)CC2)cc1] |
| 16 | 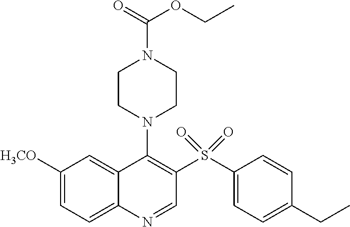 [SMILES: CCOC(=O)N1CCN(c2c(S(=O)(=O)c3ccc(CC)cc3)cnc3ccc(OC)cc23)CC1] |
| 17 | 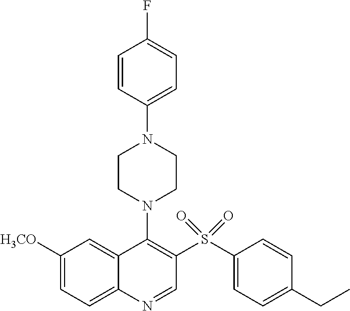 [SMILES: CCc1ccc(S(=O)(=O)c2cnc3ccc(OC)cc3c2N2CCN(c3ccc(F)cc3)CC2)cc1] |
| 18 | 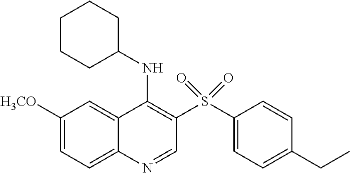 [SMILES: CCc1ccc(S(=O)(=O)c2cnc3ccc(OC)cc3c2NC2CCCCC2)cc1] |
| 19 | 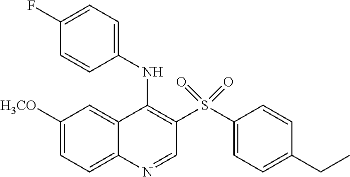 [SMILES: CCc1ccc(S(=O)(=O)c2cnc3ccc(OC)cc3c2Nc2ccc(F)cc2)cc1] |
| 20 | 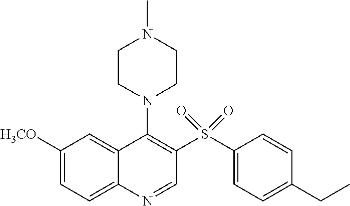 [SMILES: CCc1ccc(S(=O)(=O)c2cnc3ccc(OC)cc3c2N2CCN(C)CC2)cc1] |
| 21 | 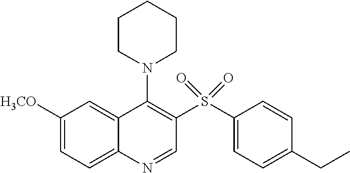 [SMILES: CCc1ccc(S(=O)(=O)c2cnc3ccc(OC)cc3c2N2CCCCC2)cc1] |
| 22 | 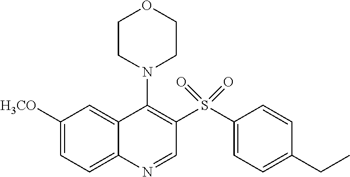 [SMILES: CCc1ccc(S(=O)(=O)c2cnc3ccc(OC)cc3c2N2CCOCC2)cc1] |
| 23 | 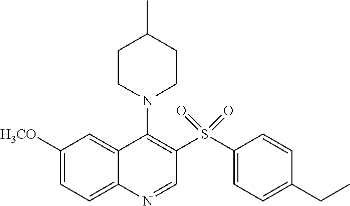 [SMILES: CCc1ccc(S(=O)(=O)c2cnc3ccc(OC)cc3c2N2CCC(C)CC2)cc1] |
| 24 | 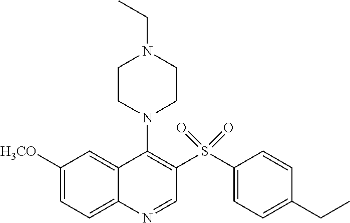 [SMILES: CCc1ccc(S(=O)(=O)c2cnc3ccc(OC)cc3c2N2CCN(CC)CC2)cc1] |
| 25 | 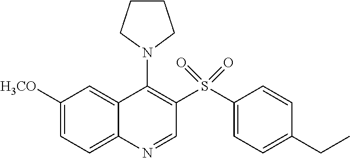 [SMILES: CCc1ccc(S(=O)(=O)c2cnc3ccc(OC)cc3c2N2CCCC2)cc1] |
| 26 | 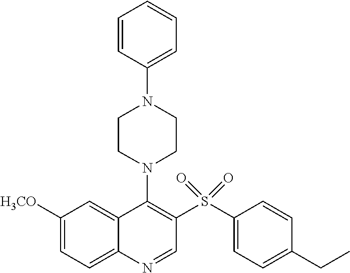 [SMILES: CCc1ccc(S(=O)(=O)c2cnc3ccc(OC)cc3c2N2CCN(c3ccccc3)CC2)cc1] |
| 27 | 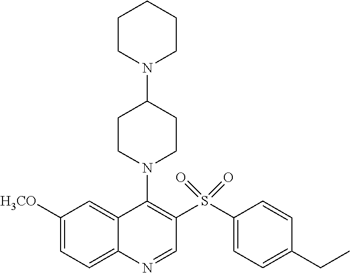 [SMILES: CCc1ccc(S(=O)(=O)c2cnc3ccc(OC)cc3c2N2CCC(N3CCCCC3)CC2)cc1] |
| 28 | 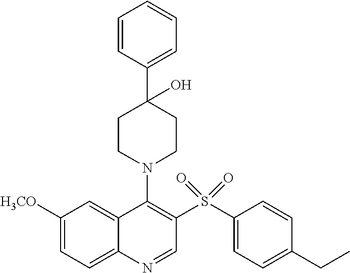 [SMILES: CCc1ccc(S(=O)(=O)c2cnc3ccc(OC)cc3c2N2CCC(O)(c3ccccc3)CC2)cc1] |
| 29 | 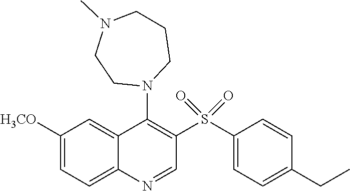 [SMILES: CCc1ccc(S(=O)(=O)c2cnc3ccc(OC)cc3c2N2CCCN(C)CC2)cc1] |
| 30 | 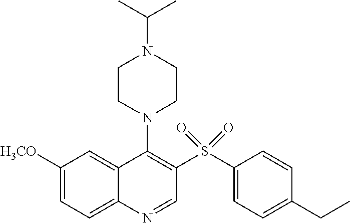 [SMILES: CCc1ccc(S(=O)(=O)c2cnc3ccc(OC)cc3c2N2CCN(C(C)C)CC2)cc1] |
| 31 | 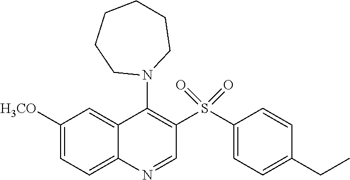 [SMILES: CCc1ccc(S(=O)(=O)c2cnc3ccc(OC)cc3c2N2CCCCCC2)cc1] |
| 32 | 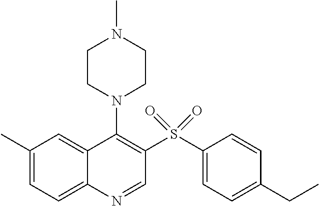 [SMILES: CCc1ccc(S(=O)(=O)c2cnc3ccc(C)cc3c2N2CCN(C)CC2)cc1] |
| 33 | 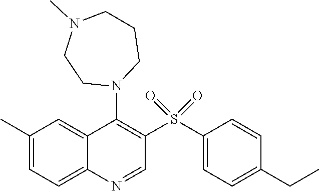 [SMILES: CCc1ccc(S(=O)(=O)c2cnc3ccc(C)cc3c2N2CCCN(C)CC2)cc1] |
| 34 | 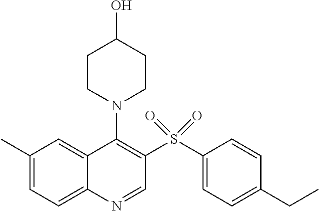 [SMILES: CCc1ccc(S(=O)(=O)c2cnc3ccc(C)cc3c2N2CCC(O)CC2)cc1] |
| 35 | 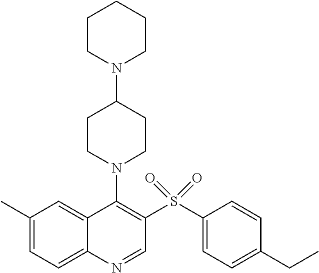 [SMILES: CCc1ccc(S(=O)(=O)c2cnc3ccc(C)cc3c2N2CCC(N3CCCCC3)CC2)cc1] |
| 36 | 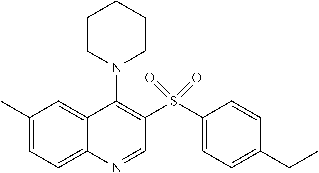 [SMILES: CCc1ccc(S(=O)(=O)c2cnc3ccc(C)cc3c2N2CCCCC2)cc1] |
| 37 | 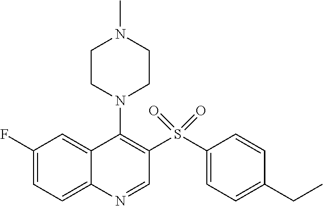 [SMILES: CCc1ccc(S(=O)(=O)c2cnc3ccc(F)cc3c2N2CCN(C)CC2)cc1] |
| 38 | 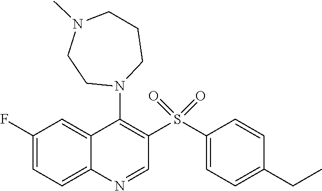 [SMILES: CCc1ccc(S(=O)(=O)c2cnc3ccc(F)cc3c2N2CCCN(C)CC2)cc1] |
| 39 | 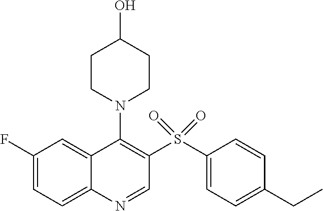 [SMILES: CCc1ccc(S(=O)(=O)c2cnc3ccc(F)cc3c2N2CCC(O)CC2)cc1] |
| 40 | 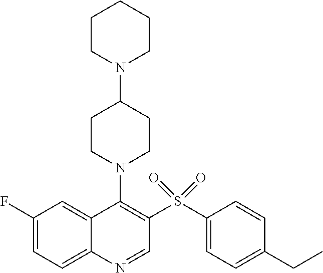 [SMILES: CCc1ccc(S(=O)(=O)c2cnc3ccc(F)cc3c2N2CCC(N3CCCCC3)CC2)cc1] |
| 41 | 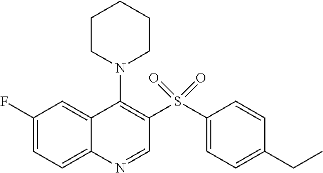 [SMILES: CCc1ccc(S(=O)(=O)c2cnc3ccc(F)cc3c2N2CCCCC2)cc1] |
| 42 | 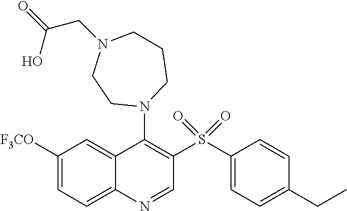 [SMILES: CCc1ccc(S(=O)(=O)c2cnc3ccc(OC(F)(F)F)cc3c2N2CCCN(CC(=O)O)CC2)cc1] |
| 43 | 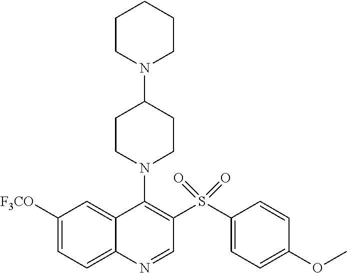 [SMILES: COc1ccc(S(=O)(=O)c2cnc3ccc(OC(F)(F)F)cc3c2N2CCC(N3CCCCC3)CC2)cc1] |
| 44 | 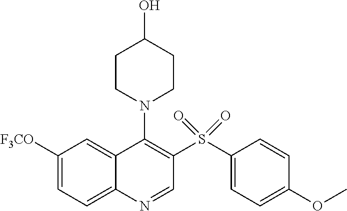 [SMILES: COc1ccc(S(=O)(=O)c2cnc3ccc(OC(F)(F)F)cc3c2N2CCC(O)CC2)cc1] |
| 45 | 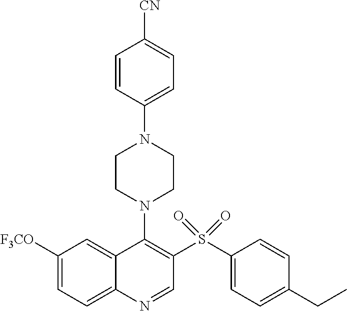 [SMILES: CCc1ccc(S(=O)(=O)c2cnc3ccc(OC(F)(F)F)cc3c2N2CCN(c3ccc(C#N)cc3)CC2)cc1] |
| 46 | 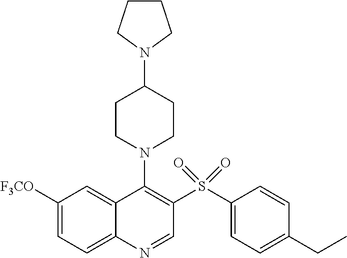 [SMILES: CCc1ccc(S(=O)(=O)c2cnc3ccc(OC(F)(F)F)cc3c2N2CCC(N3CCCC3)CC2)cc1] |
| 47 | 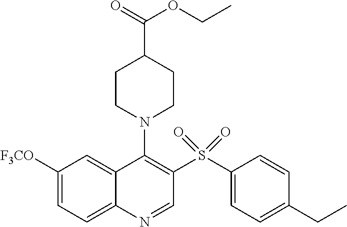 [SMILES: CCOC(=O)C1CCN(c2c(S(=O)(=O)c3ccc(CC)cc3)cnc3ccc(OC(F)(F)F)cc23)CC1] |
| 48 | 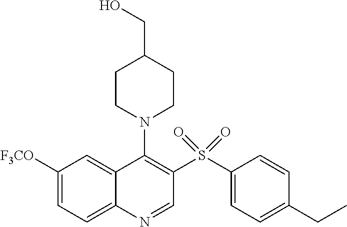 [SMILES: CCc1ccc(S(=O)(=O)c2cnc3ccc(OC(F)(F)F)cc3c2N2CCC(CO)CC2)cc1] |
| 49 | 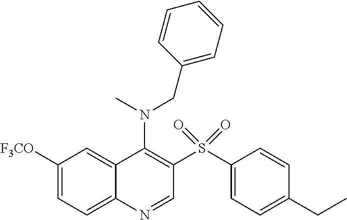 [SMILES: CCc1ccc(S(=O)(=O)c2cnc3ccc(OC(F)(F)F)cc3c2N(C)Cc2ccccc2)cc1] |
| 50 | 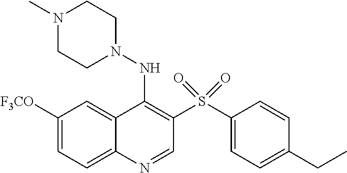 [SMILES: CCc1ccc(S(=O)(=O)c2cnc3ccc(OC(F)(F)F)cc3c2NN2CCN(C)CC2)cc1] |
| 51 | 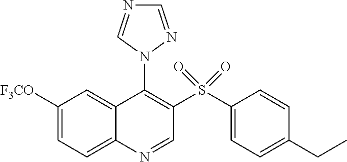 [SMILES: CCc1ccc(S(=O)(=O)c2cnc3ccc(OC(F)(F)F)cc3c2-n2cncn2)cc1] |
| 52 | 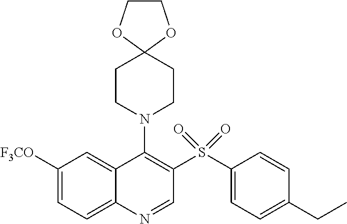 [SMILES: CCc1ccc(S(=O)(=O)c2cnc3ccc(OC(F)(F)F)cc3c2N2CCC3(CC2)OCCO3)cc1] |
| 53 | 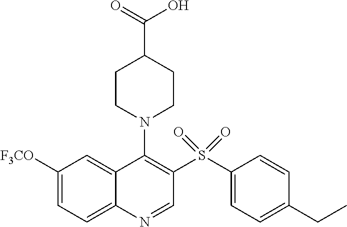 [SMILES: CCc1ccc(S(=O)(=O)c2cnc3ccc(OC(F)(F)F)cc3c2N2CCC(C(=O)O)CC2)cc1] |
| 54 | 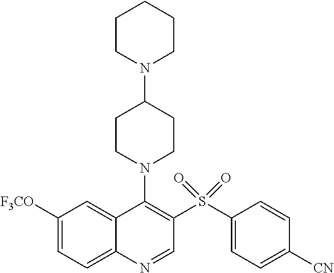 [SMILES: N#Cc1ccc(S(=O)(=O)c2cnc3ccc(OC(F)(F)F)cc3c2N2CCC(N3CCCCC3)CC2)cc1] |
| 55 | 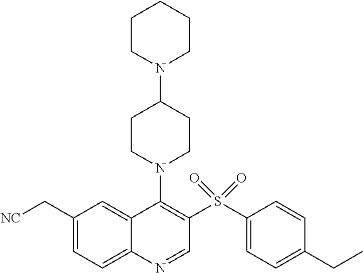 [SMILES: CCc1ccc(S(=O)(=O)c2cnc3ccc(CC#N)cc3c2N2CCC(N3CCCCC3)CC2)cc1] |
| 56 | 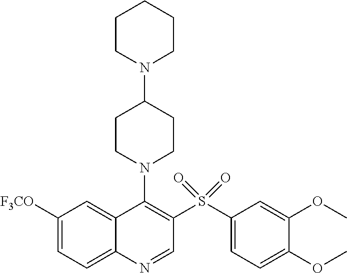 [SMILES: COc1ccc(S(=O)(=O)c2cnc3ccc(OC(F)(F)F)cc3c2N2CCC(N3CCCCC3)CC2)cc1OC] |
| 57 | 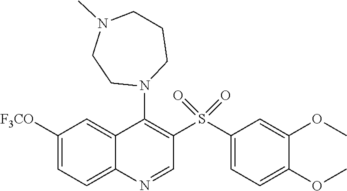 [SMILES: COc1ccc(S(=O)(=O)c2cnc3ccc(OC(F)(F)F)cc3c2N2CCCN(C)CC2)cc1OC] |
| 58 | 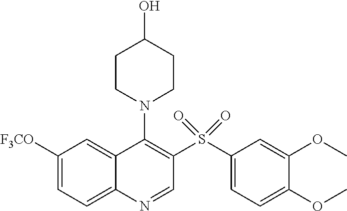 [SMILES: COc1ccc(S(=O)(=O)c2cnc3ccc(OC(F)(F)F)cc3c2N2CCC(O)CC2)cc1OC] |
| 59 | 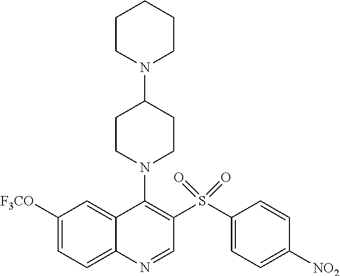 [SMILES: O=[N+]([O-])c1ccc(S(=O)(=O)c2cnc3ccc(OC(F)(F)F)cc3c2N2CCC(N3CCCCC3)CC2)cc1] |
| 60 | 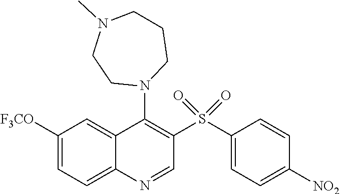 [SMILES: CN1CCCN(c2c(S(=O)(=O)c3ccc([N+](=O)[O-])cc3)cnc3ccc(OC(F)(F)F)cc23)CC1] |
| 61 | 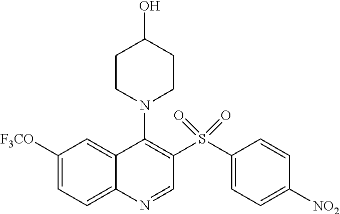 [SMILES: O=[N+]([O-])c1ccc(S(=O)(=O)c2cnc3ccc(OC(F)(F)F)cc3c2N2CCC(O)CC2)cc1] |
| 62 | 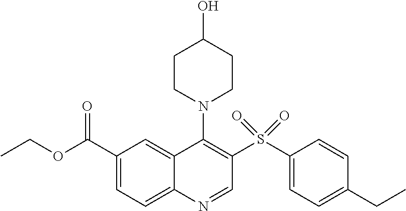 [SMILES: CCOC(=O)c1ccc2ncc(S(=O)(=O)c3ccc(CC)cc3)c(N3CCC(O)CC3)c2c1] |
| 63 | 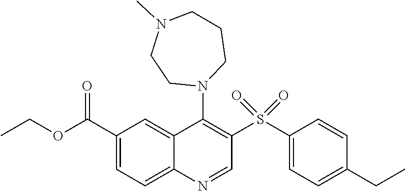 [SMILES: CCOC(=O)c1ccc2ncc(S(=O)(=O)c3ccc(CC)cc3)c(N3CCCN(C)CC3)c2c1] |
| 64 | 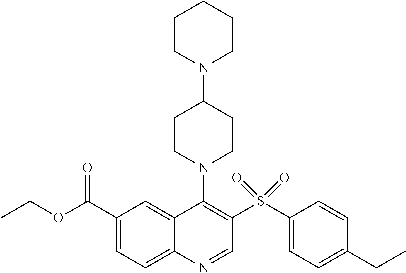 [SMILES: CCOC(=O)c1ccc2ncc(S(=O)(=O)c3ccc(CC)cc3)c(N3CCC(N4CCCCC4)CC3)c2c1] |
| 65 | 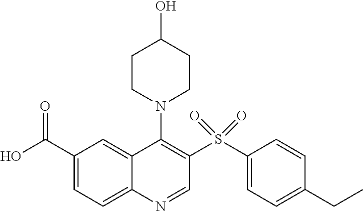 [SMILES: CCc1ccc(S(=O)(=O)c2cnc3ccc(C(=O)O)cc3c2N2CCC(O)CC2)cc1] |
| 66 | 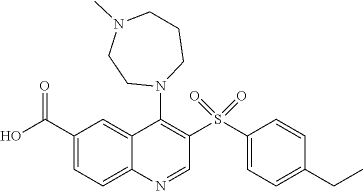 [SMILES: CCc1ccc(S(=O)(=O)c2cnc3ccc(C(=O)O)cc3c2N2CCCN(C)CC2)cc1] |
| 67 | 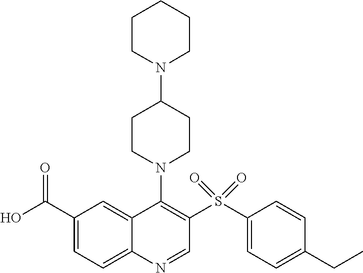 [SMILES: CCc1ccc(S(=O)(=O)c2cnc3ccc(C(=O)O)cc3c2N2CCC(N3CCCCC3)CC2)cc1] |
| 68 | 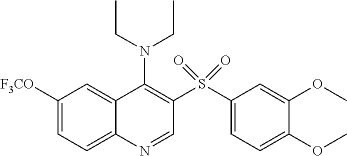 [SMILES: CCN(CC)c1c(S(=O)(=O)c2ccc(OC)c(OC)c2)cnc2ccc(OC(F)(F)F)cc12] |
| 69 | 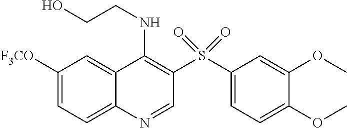 [SMILES: COc1ccc(S(=O)(=O)c2cnc3ccc(OC(F)(F)F)cc3c2NCCO)cc1OC] |
| 70 | 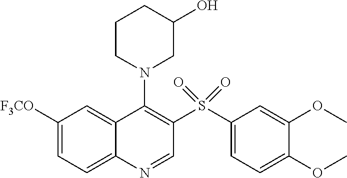 [SMILES: COc1ccc(S(=O)(=O)c2cnc3ccc(OC(F)(F)F)cc3c2N2CCCC(O)C2)cc1OC] |
| 71 | 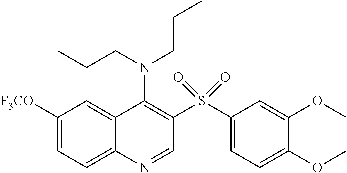 [SMILES: CCCN(CCC)c1c(S(=O)(=O)c2ccc(OC)c(OC)c2)cnc2ccc(OC(F)(F)F)cc12] |
| 72 | 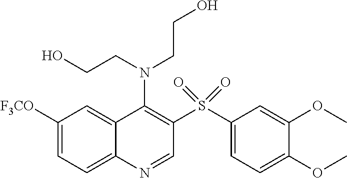 [SMILES: COc1ccc(S(=O)(=O)c2cnc3ccc(OC(F)(F)F)cc3c2N(CCO)CCO)cc1OC] |
| 73 | 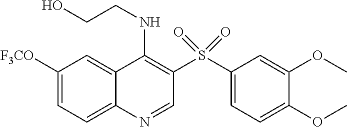 [SMILES: COc1ccc(S(=O)(=O)c2cnc3ccc(OC(F)(F)F)cc3c2NCCO)cc1OC] |
| 74 | 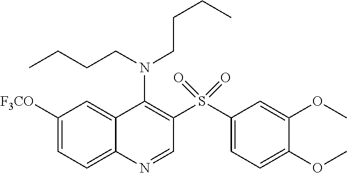 [SMILES: CCCCN(CCCC)c1c(S(=O)(=O)c2ccc(OC)c(OC)c2)cnc2ccc(OC(F)(F)F)cc12] |
| 75 | 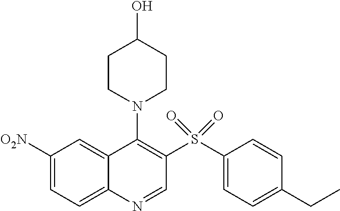 [SMILES: CCc1ccc(S(=O)(=O)c2cnc3ccc([N+](=O)[O-])cc3c2N2CCC(O)CC2)cc1] |
| 76 | 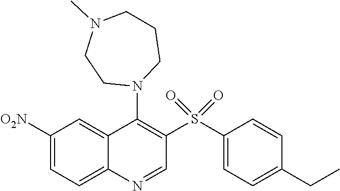 [SMILES: CCc1ccc(S(=O)(=O)c2cnc3ccc([N+](=O)[O-])cc3c2N2CCCN(C)CC2)cc1] |
| 77 | 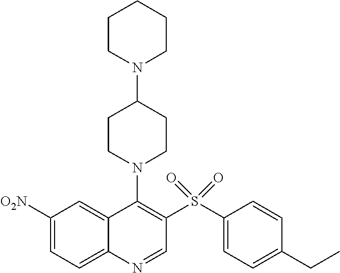 [SMILES: CCc1ccc(S(=O)(=O)c2cnc3ccc([N+](=O)[O-])cc3c2N2CCC(N3CCCCC3)CC2)cc1] |
| 78 | 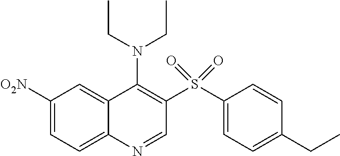 [SMILES: CCc1ccc(S(=O)(=O)c2cnc3ccc([N+](=O)[O-])cc3c2N(CC)CC)cc1] |
| 79 | 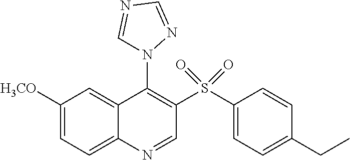 [SMILES: CCc1ccc(S(=O)(=O)c2cnc3ccc(OC)cc3c2-n2cncn2)cc1] |
| 80 | 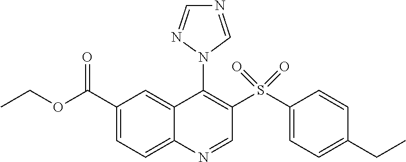 [SMILES: CCOC(=O)c1ccc2ncc(S(=O)(=O)c3ccc(CC)cc3)c(-n3cncn3)c2c1] |
| 81 | 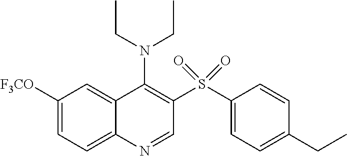 [SMILES: CCc1ccc(S(=O)(=O)c2cnc3ccc(OC(F)(F)F)cc3c2N(CC)CC)cc1] |
| 82 | 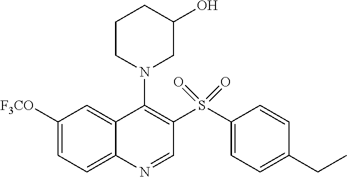 [SMILES: CCc1ccc(S(=O)(=O)c2cnc3ccc(OC(F)(F)F)cc3c2N2CCCC(O)C2)cc1] |
| 83 | 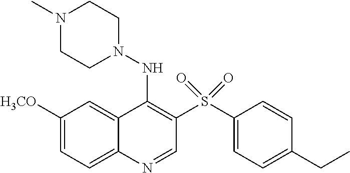 [SMILES: CCc1ccc(S(=O)(=O)c2cnc3ccc(OC)cc3c2NN2CCN(C)CC2)cc1] |
| 84 | 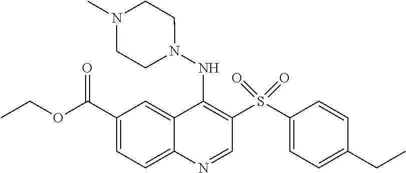 [SMILES: CCOC(=O)c1ccc2ncc(S(=O)(=O)c3ccc(CC)cc3)c(NN3CCN(C)CC3)c2c1] |
| 85 | 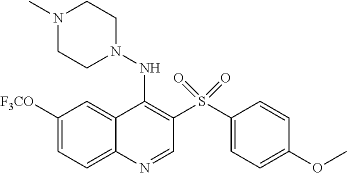 [SMILES: COc1ccc(S(=O)(=O)c2cnc3ccc(OC(F)(F)F)cc3c2NN2CCN(C)CC2)cc1] |
| 86 | 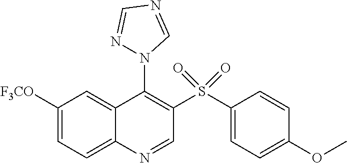 [SMILES: COc1ccc(S(=O)(=O)c2cnc3ccc(OC(F)(F)F)cc3c2-n2cncn2)cc1] |
| 87 | 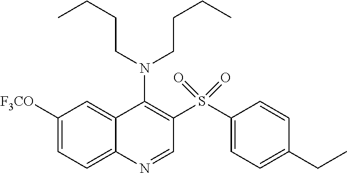 [SMILES: CCCCN(CCCC)c1c(S(=O)(=O)c2ccc(CC)cc2)cnc2ccc(OC(F)(F)F)cc12] |
| 88 | 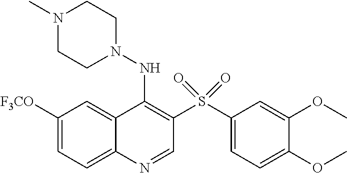 [SMILES: COc1ccc(S(=O)(=O)c2cnc3ccc(OC(F)(F)F)cc3c2NN2CCN(C)CC2)cc1OC] |
| 89 | 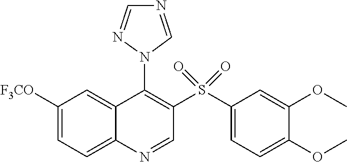 [SMILES: COc1ccc(S(=O)(=O)c2cnc3ccc(OC(F)(F)F)cc3c2-n2cncn2)cc1OC] |
| 90 | 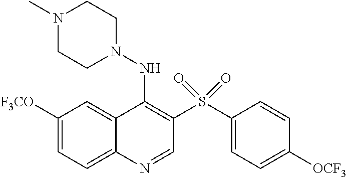 [SMILES: CN1CCN(Nc2c(S(=O)(=O)c3ccc(OC(F)(F)F)cc3)cnc3ccc(OC(F)(F)F)cc23)CC1] |
| 91 | 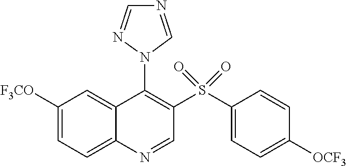 [SMILES: O=S(=O)(c1ccc(OC(F)(F)F)cc1)c1cnc2ccc(OC(F)(F)F)cc2c1-n1cncn1] |
| 92 | 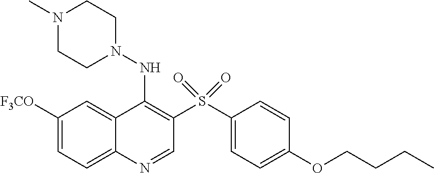 [SMILES: CCCCOc1ccc(S(=O)(=O)c2cnc3ccc(OC(F)(F)F)cc3c2NN2CCN(C)CC2)cc1] |
| 93 | 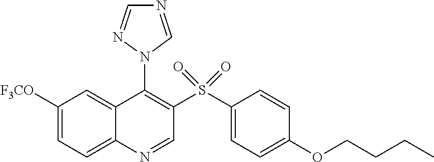 [SMILES: CCCCOc1ccc(S(=O)(=O)c2cnc3ccc(OC(F)(F)F)cc3c2-n2cncn2)cc1] |
| 94 | 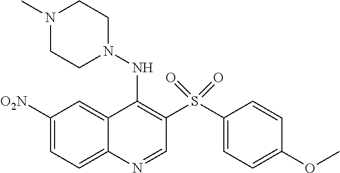 |
| 95 | 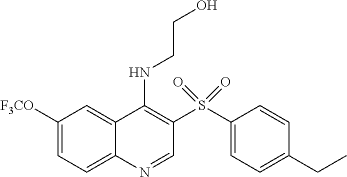 [SMILES: CCc1ccc(S(=O)(=O)c2cnc3ccc(OC(F)(F)F)cc3c2NCCO)cc1] |
| 96 | 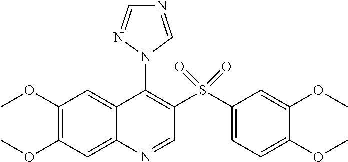 |
| 97 | 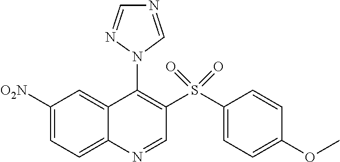 [SMILES: COc1ccc(S(=O)(=O)c2cnc3ccc([N+](=O)[O-])cc3c2-n2cncn2)cc1] |
| 98 | 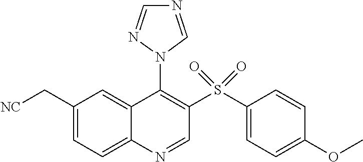 [SMILES: COc1ccc(S(=O)(=O)c2cnc3ccc(CC#N)cc3c2-n2cncn2)cc1] |
| 99 | 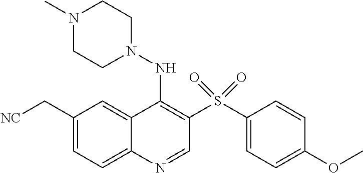 [SMILES: COc1ccc(S(=O)(=O)c2cnc3ccc(CC#N)cc3c2NN2CCN(C)CC2)cc1] |
| 100 | 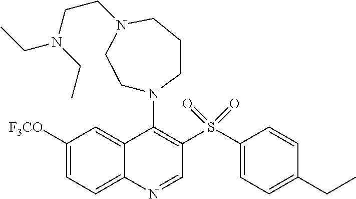 [SMILES: CCc1ccc(S(=O)(=O)c2cnc3ccc(OC(F)(F)F)cc3c2N2CCCN(CCN(CC)CC)CC2)cc1] |
| 101 | 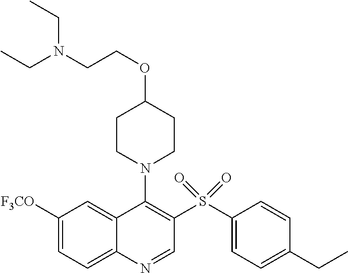 [SMILES: CCc1ccc(S(=O)(=O)c2cnc3ccc(OC(F)(F)F)cc3c2N2CCC(OCCN(CC)CC)CC2)cc1] |
| 102 | 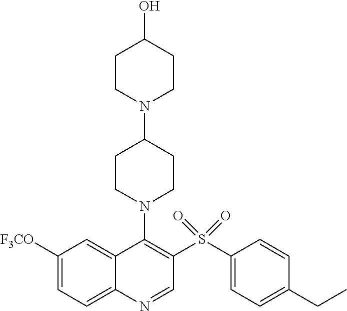 [SMILES: CCc1ccc(S(=O)(=O)c2cnc3ccc(OC(F)(F)F)cc3c2N2CCC(N3CCC(O)CC3)CC2)cc1] |
| 103 | 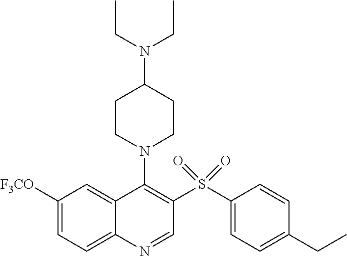 [SMILES: CCc1ccc(S(=O)(=O)c2cnc3ccc(OC(F)(F)F)cc3c2N2CCC(N(CC)CC)CC2)cc1] |
| 104 | 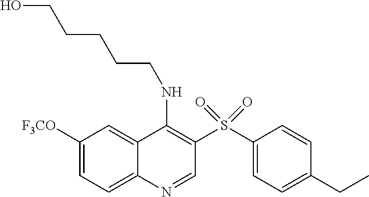 [SMILES: CCc1ccc(S(=O)(=O)c2cnc3ccc(OC(F)(F)F)cc3c2NCCCCCO)cc1] |
| 105 | 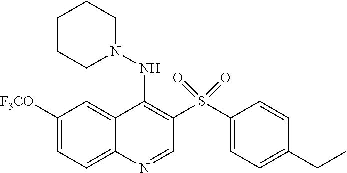 [SMILES: CCc1ccc(S(=O)(=O)c2cnc3ccc(OC(F)(F)F)cc3c2NN2CCCCC2)cc1] |
| 106 | 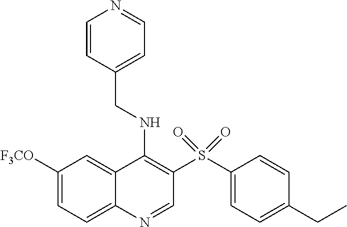 [SMILES: CCc1ccc(S(=O)(=O)c2cnc3ccc(OC(F)(F)F)cc3c2NCc2ccncc2)cc1] |
| 107 | 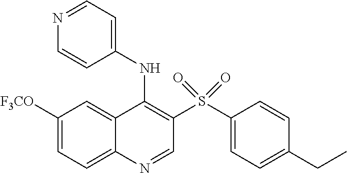 [SMILES: CCc1ccc(S(=O)(=O)c2cnc3ccc(OC(F)(F)F)cc3c2Nc2ccncc2)cc1] |
| 108 | 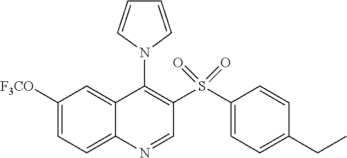 [SMILES: CCc1ccc(S(=O)(=O)c2cnc3ccc(OC(F)(F)F)cc3c2-n2cccc2)cc1] |
| 109 | 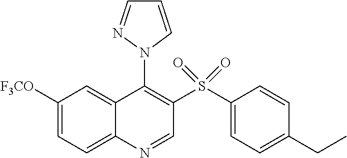 [SMILES: CCc1ccc(S(=O)(=O)c2cnc3ccc(OC(F)(F)F)cc3c2-n2cccn2)cc1] |
| 110 | 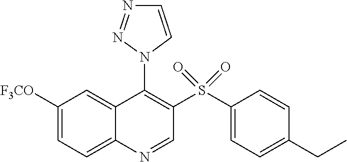 [SMILES: CCc1ccc(S(=O)(=O)c2cnc3ccc(OC(F)(F)F)cc3c2-n2ccnn2)cc1] |
| 111 | 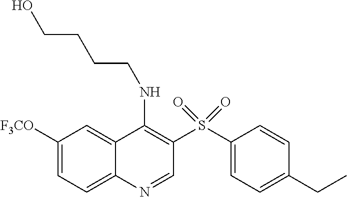 [SMILES: CCc1ccc(S(=O)(=O)c2cnc3ccc(OC(F)(F)F)cc3c2NCCCCO)cc1] |
| 112 | 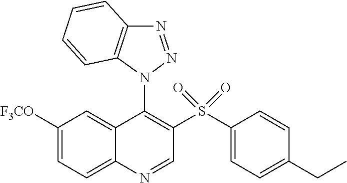 [SMILES: CCc1ccc(S(=O)(=O)c2cnc3ccc(OC(F)(F)F)cc3c2-n2nnc3ccccc32)cc1] |
| 113 | 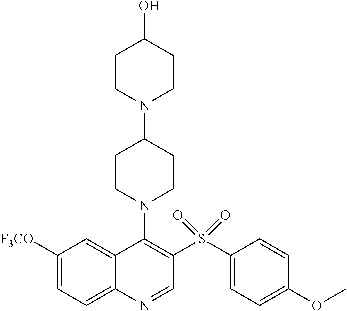 [SMILES: COc1ccc(S(=O)(=O)c2cnc3ccc(OC(F)(F)F)cc3c2N2CCC(N3CCC(O)CC3)CC2)cc1] |
| 114 | 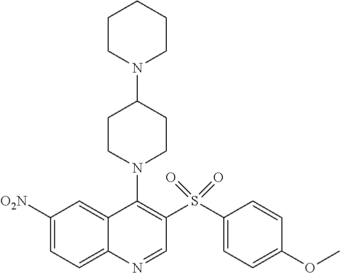 [SMILES: COc1ccc(S(=O)(=O)c2cnc3ccc([N+](=O)[O-])cc3c2N2CCC(N3CCCCC3)CC2)cc1] |
| 115 | 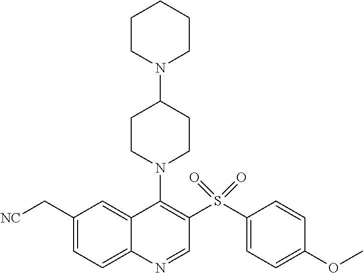 [SMILES: COc1ccc(S(=O)(=O)c2cnc3ccc(CC#N)cc3c2N2CCC(N3CCCCC3)CC2)cc1] |
| 116 | 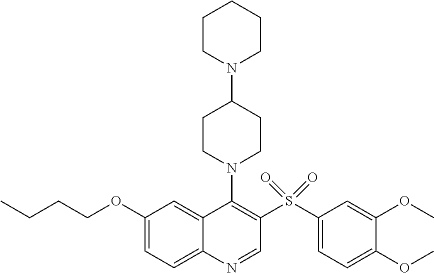 [SMILES: CCCCOc1ccc2ncc(S(=O)(=O)c3ccc(OC)c(OC)c3)c(N3CCC(N4CCCCC4)CC3)c2c1] |
| 117 | 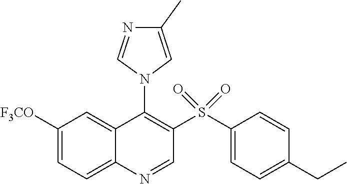 [SMILES: CCc1ccc(S(=O)(=O)c2cnc3ccc(OC(F)(F)F)cc3c2-n2cnc(C)c2)cc1] |
| 118 | 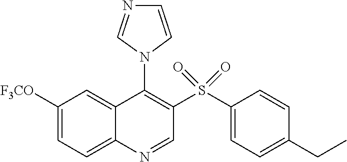 [SMILES: CCc1ccc(S(=O)(=O)c2cnc3ccc(OC(F)(F)F)cc3c2-n2ccnc2)cc1] |
| 119 | 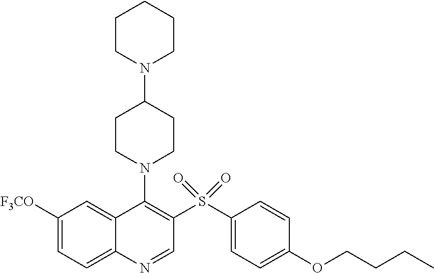 [SMILES: CCCCOc1ccc(S(=O)(=O)c2cnc3ccc(OC(F)(F)F)cc3c2N2CCC(N3CCCCC3)CC2)cc1] |
| 120 | 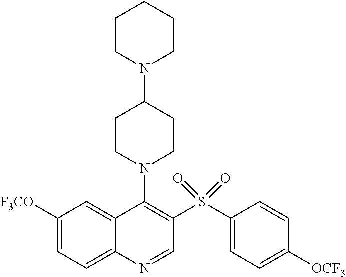 [SMILES: O=S(=O)(c1ccc(OC(F)(F)F)cc1)c1cnc2ccc(OC(F)(F)F)cc2c1N1CCC(N2CCCCC2)CC1] |
| 121 | 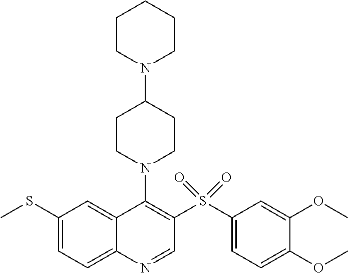 |
| 122 | 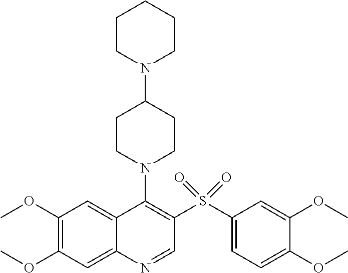 |
| 123 | 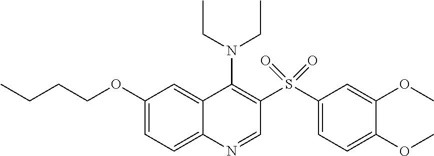 [SMILES: CCCCOc1ccc2ncc(S(=O)(=O)c3ccc(OC)c(OC)c3)c(N(CC)CC)c2c1] |
| 124 | 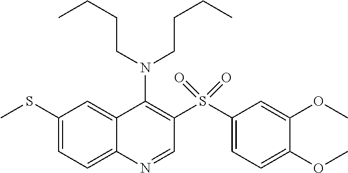 |
| 125 | 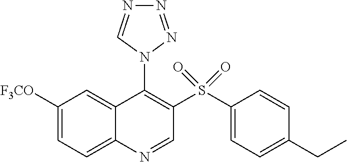 [SMILES: CCc1ccc(S(=O)(=O)c2cnc3ccc(OC(F)(F)F)cc3c2-n2cnnn2)cc1] |
| 126 | 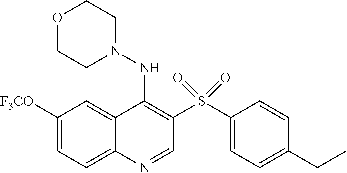 [SMILES: CCc1ccc(S(=O)(=O)c2cnc3ccc(OC(F)(F)F)cc3c2NN2CCOCC2)cc1] |
| 127 | 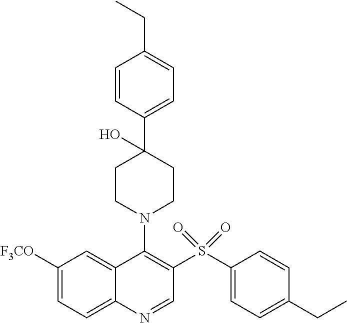 [SMILES: CCc1ccc(C2(O)CCN(c3c(S(=O)(=O)c4ccc(CC)cc4)cnc4ccc(OC(F)(F)F)cc34)CC2)cc1] |
| 128 | 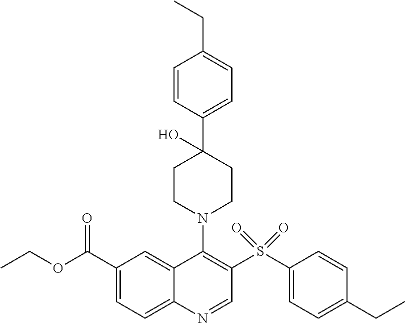 [SMILES: CCOC(=O)c1ccc2ncc(S(=O)(=O)c3ccc(CC)cc3)c(N3CCC(O)(c4ccc(CC)cc4)CC3)c2c1] |
| 129 | 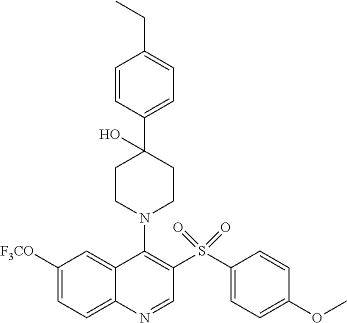 [SMILES: CCc1ccc(C2(O)CCN(c3c(S(=O)(=O)c4ccc(OC)cc4)cnc4ccc(OC(F)(F)F)cc34)CC2)cc1] |
| 130 | 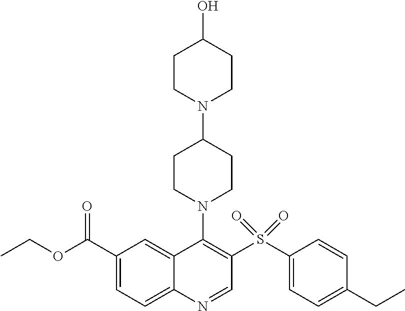 [SMILES: CCOC(=O)c1ccc2ncc(S(=O)(=O)c3ccc(CC)cc3)c(N3CCC(N4CCC(O)CC4)CC3)c2c1] |
| 131 | 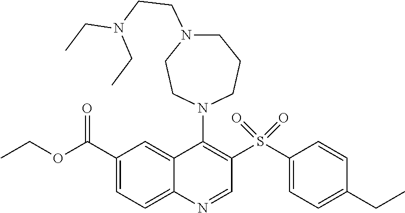 [SMILES: CCOC(=O)c1ccc2ncc(S(=O)(=O)c3ccc(CC)cc3)c(N3CCCN(CCN(CC)CC)CC3)c2c1] |
| 132 | 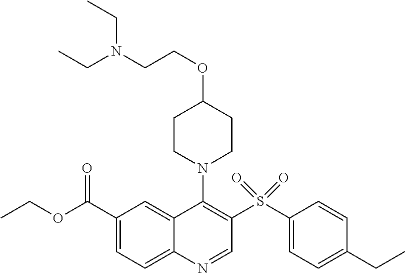 [SMILES: CCOC(=O)c1ccc2ncc(S(=O)(=O)c3ccc(CC)cc3)c(N3CCC(OCCN(CC)CC)CC3)c2c1] |
| 133 | 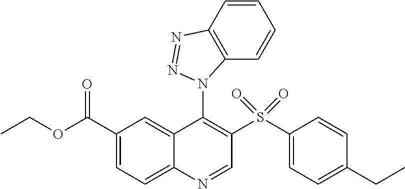 [SMILES: CCOC(=O)c1ccc2ncc(S(=O)(=O)c3ccc(CC)cc3)c(-n3nnc4ccccc43)c2c1] |
| 134 | 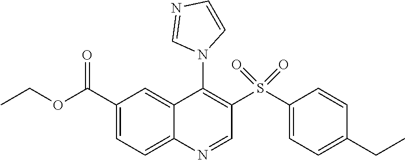 [SMILES: CCOC(=O)c1ccc2ncc(S(=O)(=O)c3ccc(CC)cc3)c(-n3ccnc3)c2c1] |
| 135 | 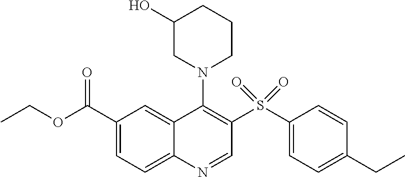 [SMILES: CCOC(=O)c1ccc2ncc(S(=O)(=O)c3ccc(CC)cc3)c(N3CCCC(O)C3)c2c1] |
| 136 | 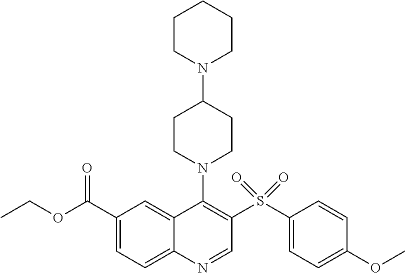 [SMILES: CCOC(=O)c1ccc2ncc(S(=O)(=O)c3ccc(OC)cc3)c(N3CCC(N4CCCCC4)CC3)c2c1] |
| 137 | 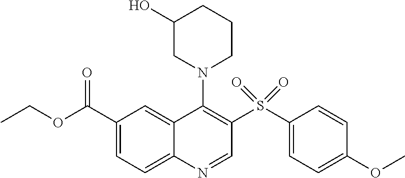 [SMILES: CCOC(=O)c1ccc2ncc(S(=O)(=O)c3ccc(OC)cc3)c(N3CCCC(O)C3)c2c1] |
| 138 | 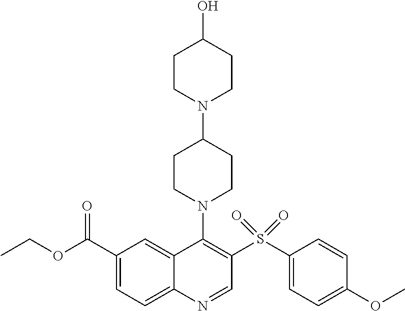 [SMILES: CCOC(=O)c1ccc2ncc(S(=O)(=O)c3ccc(OC)cc3)c(N3CCC(N4CCC(O)CC4)CC3)c2c1] |
| 139 | 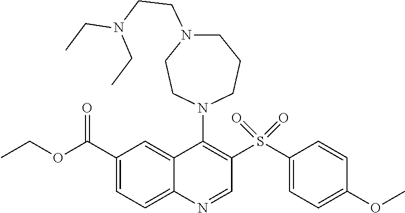 [SMILES: CCOC(=O)c1ccc2ncc(S(=O)(=O)c3ccc(OC)cc3)c(N3CCCN(CCN(CC)CC)CC3)c2c1] |
| 140 | 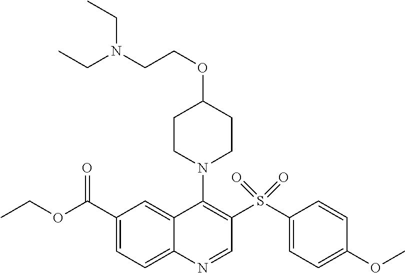 [SMILES: CCOC(=O)c1ccc2ncc(S(=O)(=O)c3ccc(OC)cc3)c(N3CCC(OCCN(CC)CC)CC3)c2c1] |
| 141 | 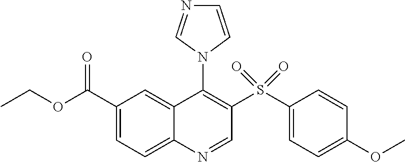 [SMILES: CCOC(=O)c1ccc2ncc(S(=O)(=O)c3ccc(OC)cc3)c(-n3ccnc3)c2c1] |
| 142 | 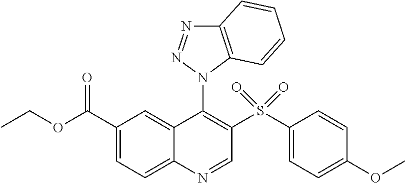 [SMILES: CCOC(=O)c1ccc2ncc(S(=O)(=O)c3ccc(OC)cc3)c(-n3nnc4ccccc43)c2c1] |
| 143 | 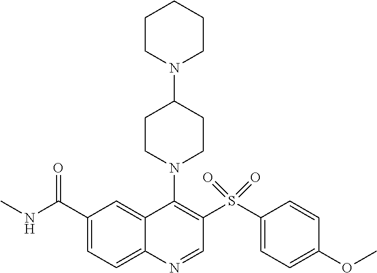 |
| 144 | 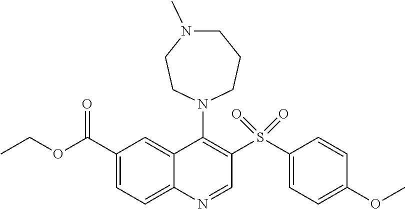 [SMILES: CCOC(=O)c1ccc2ncc(S(=O)(=O)c3ccc(OC)cc3)c(N3CCCN(C)CC3)c2c1] |
| 145 | 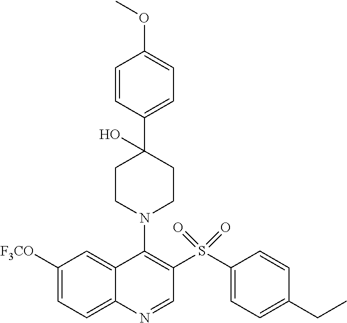 [SMILES: CCc1ccc(S(=O)(=O)c2cnc3ccc(OC(F)(F)F)cc3c2N2CCC(O)(c3ccc(OC)cc3)CC2)cc1] |
| 146 | 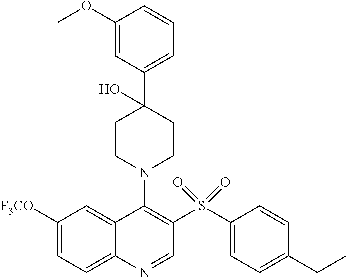 [SMILES: CCc1ccc(S(=O)(=O)c2cnc3ccc(OC(F)(F)F)cc3c2N2CCC(O)(c3cccc(OC)c3)CC2)cc1] |
| 147 | 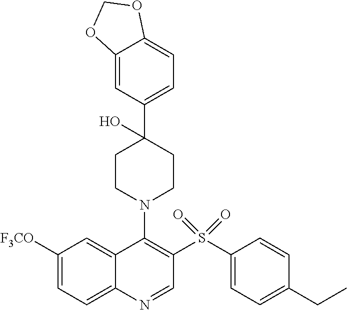 [SMILES: CCc1ccc(S(=O)(=O)c2cnc3ccc(OC(F)(F)F)cc3c2N2CCC(O)(c3ccc4c(c3)OCO4)CC2)cc1] |
| 148 | 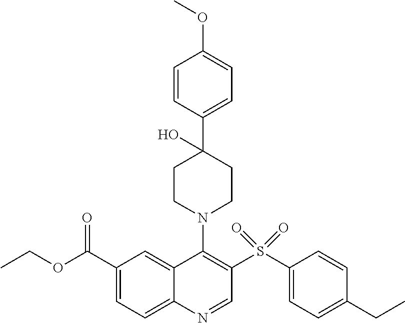 [SMILES: CCOC(=O)c1ccc2ncc(S(=O)(=O)c3ccc(CC)cc3)c(N3CCC(O)(c4ccc(OC)cc4)CC3)c2c1] |
| 149 | 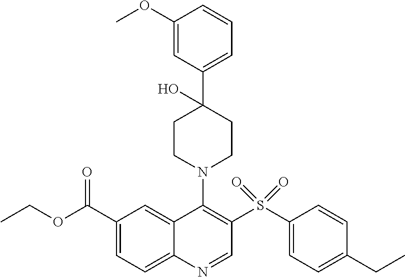 [SMILES: CCOC(=O)c1ccc2ncc(S(=O)(=O)c3ccc(CC)cc3)c(N3CCC(O)(c4cccc(OC)c4)CC3)c2c1] |
| 150 | 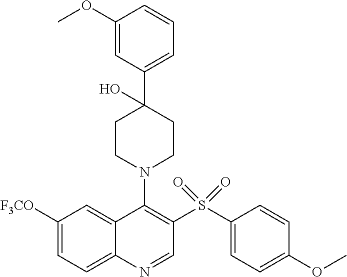 [SMILES: COc1ccc(S(=O)(=O)c2cnc3ccc(OC(F)(F)F)cc3c2N2CCC(O)(c3cccc(OC)c3)CC2)cc1] |
| 151 | 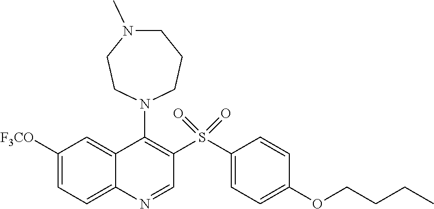 [SMILES: CCCCOc1ccc(S(=O)(=O)c2cnc3ccc(OC(F)(F)F)cc3c2N2CCCN(C)CC2)cc1] |
| 152 | 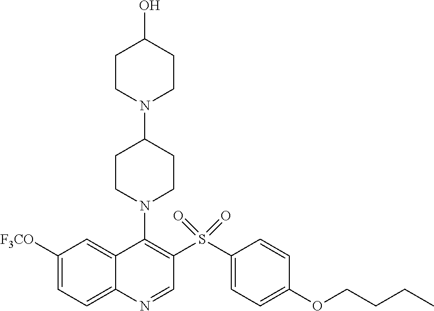 [SMILES: CCCCOc1ccc(S(=O)(=O)c2cnc3ccc(OC(F)(F)F)cc3c2N2CCC(N3CCC(O)CC3)CC2)cc1] |
| 153 | 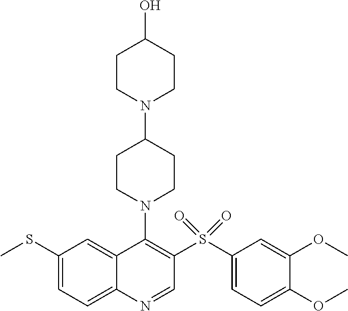 |
| 154 | 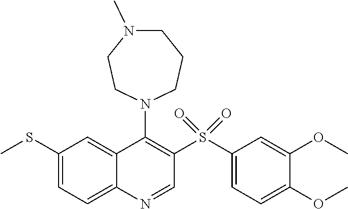 |
| 155 | 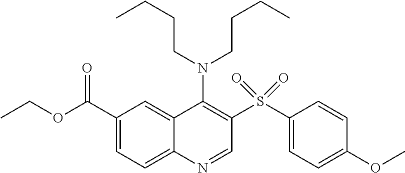 [SMILES: CCCCN(CCCC)c1c(S(=O)(=O)c2ccc(OC)cc2)cnc2ccc(C(=O)OCC)cc12] |
| 156 | 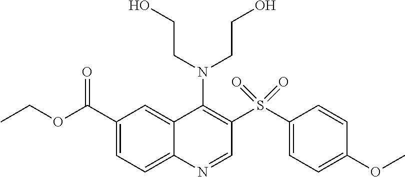 [SMILES: CCOC(=O)c1ccc2ncc(S(=O)(=O)c3ccc(OC)cc3)c(N(CCO)CCO)c2c1] |
| 157 | 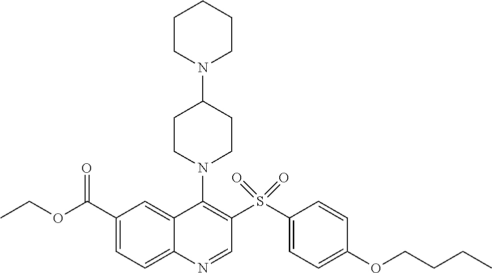 [SMILES: CCCCOc1ccc(S(=O)(=O)c2cnc3ccc(C(=O)OCC)cc3c2N2CCC(N3CCCCC3)CC2)cc1] |
| 158 | 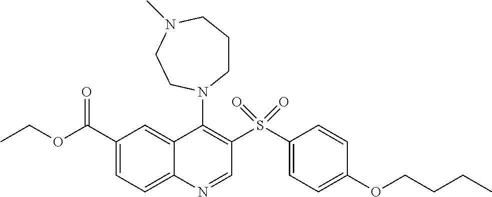 [SMILES: CCCCOc1ccc(S(=O)(=O)c2cnc3ccc(C(=O)OCC)cc3c2N2CCCN(C)CC2)cc1] |
| 159 | 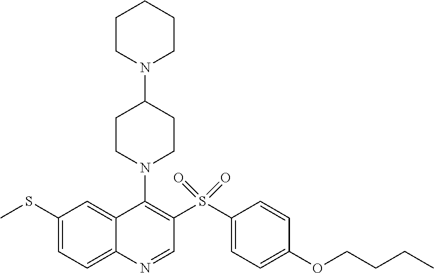 [SMILES: CCCCOc1ccc(S(=O)(=O)c2cnc3ccc(SC)cc3c2N2CCC(N3CCCCC3)CC2)cc1] |
| 160 | 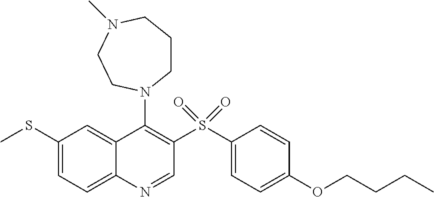 [SMILES: CCCCOc1ccc(S(=O)(=O)c2cnc3ccc(SC)cc3c2N2CCCN(C)CC2)cc1] |
| 161 | 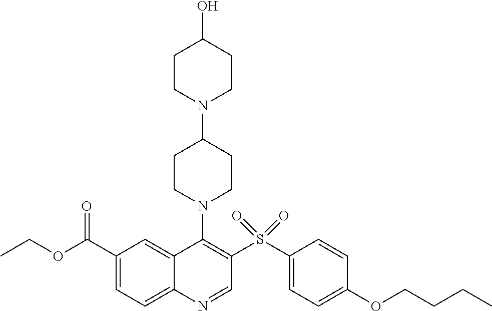 [SMILES: CCCCOc1ccc(S(=O)(=O)c2cnc3ccc(C(=O)OCC)cc3c2N2CCC(N3CCC(O)CC3)CC2)cc1] |
| 162 | 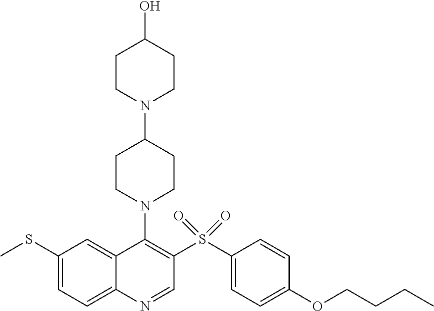 [SMILES: CCCCOc1ccc(S(=O)(=O)c2cnc3ccc(SC)cc3c2N2CCC(N3CCC(O)CC3)CC2)cc1] |
| 163 | 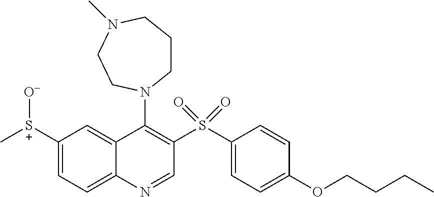 [SMILES: CCCCOc1ccc(S(=O)(=O)c2cnc3ccc([S+](C)[O-])cc3c2N2CCCN(C)CC2)cc1] |
| 164 | 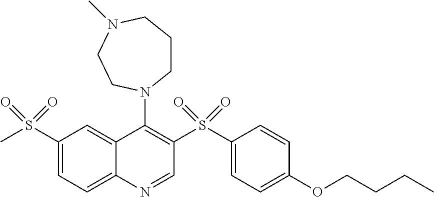 [SMILES: CCCCOc1ccc(S(=O)(=O)c2cnc3ccc(S(C)(=O)=O)cc3c2N2CCCN(C)CC2)cc1] |
| 165 | 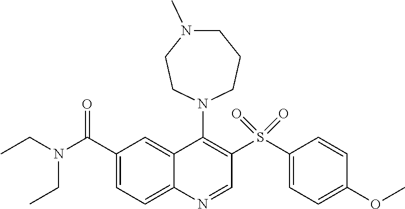 [SMILES: CCN(CC)C(=O)c1ccc2ncc(S(=O)(=O)c3ccc(OC)cc3)c(N3CCCN(C)CC3)c2c1] |
| 166 | 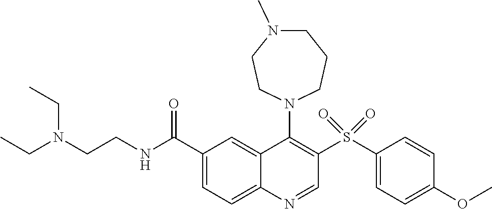 [SMILES: CCN(CC)CCNC(=O)c1ccc2ncc(S(=O)(=O)c3ccc(OC)cc3)c(N3CCCN(C)CC3)c2c1] |
| 167 | 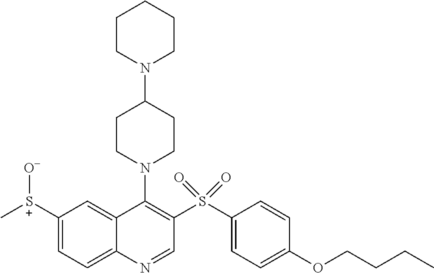 [SMILES: CCCCOc1ccc(S(=O)(=O)c2cnc3ccc([S+](C)[O-])cc3c2N2CCC(N3CCCCC3)CC2)cc1] |
| 168 | 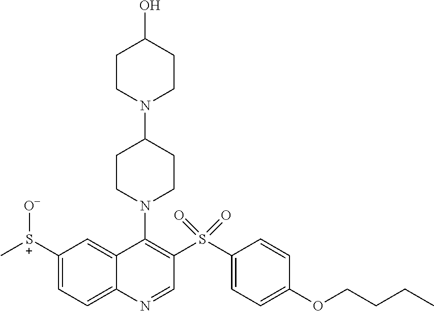 [SMILES: CCCCOc1ccc(S(=O)(=O)c2cnc3ccc([S+](C)[O-])cc3c2N2CCC(N3CCC(O)CC3)CC2)cc1] |
| 169 | 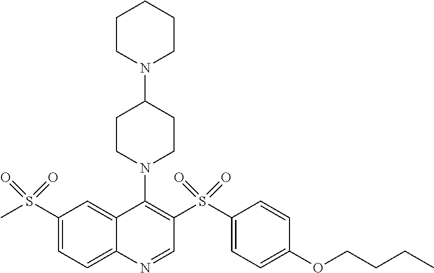 [SMILES: CCCCOc1ccc(S(=O)(=O)c2cnc3ccc(S(C)(=O)=O)cc3c2N2CCC(N3CCCCC3)CC2)cc1] |
| 170 | 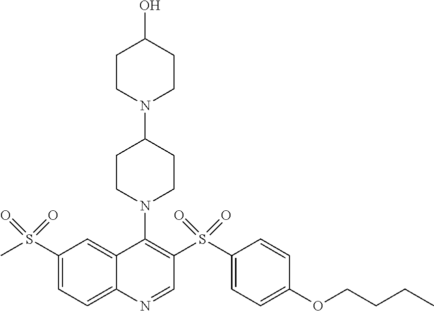 [SMILES: CCCCOc1ccc(S(=O)(=O)c2cnc3ccc(S(C)(=O)=O)cc3c2N2CCC(N3CCC(O)CC3)CC2)cc1] |
| 171 | 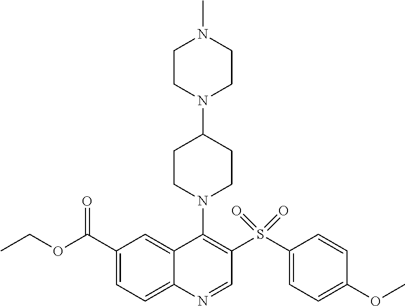 [SMILES: CCOC(=O)c1ccc2ncc(S(=O)(=O)c3ccc(OC)cc3)c(N3CCC(N4CCN(C)CC4)CC3)c2c1] |
| 172 | 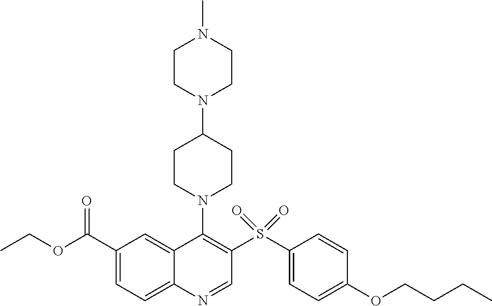 [SMILES: CCCCOc1ccc(S(=O)(=O)c2cnc3ccc(C(=O)OCC)cc3c2N2CCC(N3CCN(C)CC3)CC2)cc1] |
| 173 | 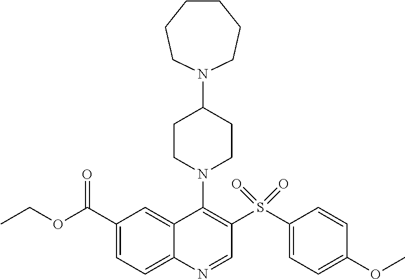 [SMILES: CCOC(=O)c1ccc2ncc(S(=O)(=O)c3ccc(OC)cc3)c(N3CCC(N4CCCCCC4)CC3)c2c1] |
| 174 | 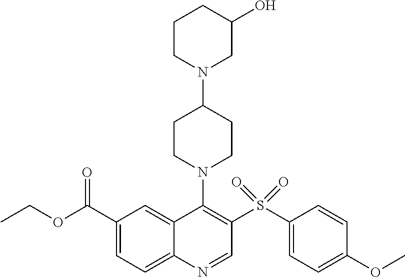 [SMILES: CCOC(=O)c1ccc2ncc(S(=O)(=O)c3ccc(OC)cc3)c(N3CCC(N4CCCC(O)C4)CC3)c2c1] |
| 175 | 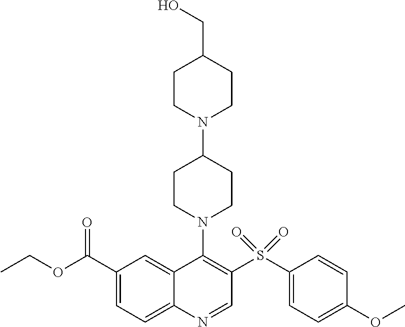 [SMILES: CCOC(=O)c1ccc2ncc(S(=O)(=O)c3ccc(OC)cc3)c(N3CCC(N4CCC(CO)CC4)CC3)c2c1] |
| 176 | 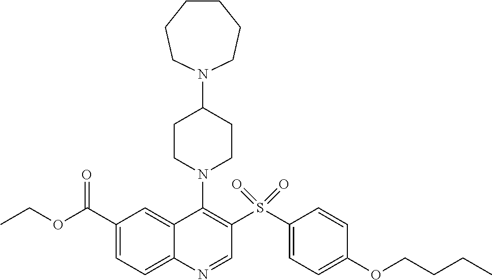 [SMILES: CCCCOc1ccc(S(=O)(=O)c2cnc3ccc(C(=O)OCC)cc3c2N2CCC(N3CCCCCC3)CC2)cc1] |
| 177 | 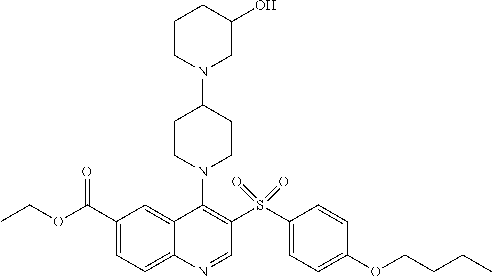 [SMILES: CCCCOc1ccc(S(=O)(=O)c2cnc3ccc(C(=O)OCC)cc3c2N2CCC(N3CCCC(O)C3)CC2)cc1] |
| 178 | 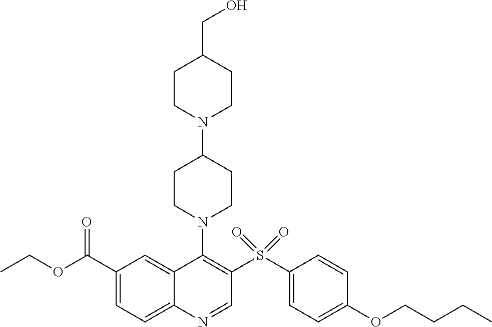 [SMILES: CCCCOc1ccc(S(=O)(=O)c2cnc3ccc(C(=O)OCC)cc3c2N2CCC(N3CCC(CO)CC3)CC2)cc1] |
| 179 | 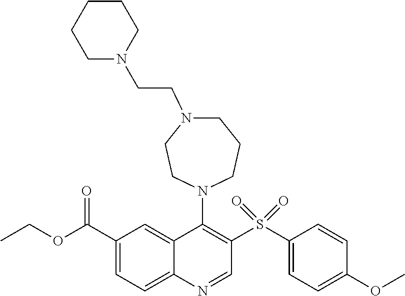 [SMILES: CCOC(=O)c1ccc2ncc(S(=O)(=O)c3ccc(OC)cc3)c(N3CCCN(CCN4CCCCC4)CC3)c2c1] |
| 180 | 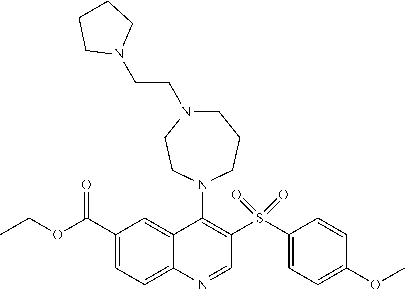 [SMILES: CCOC(=O)c1ccc2ncc(S(=O)(=O)c3ccc(OC)cc3)c(N3CCCN(CCN4CCCC4)CC3)c2c1] |
| 181 | 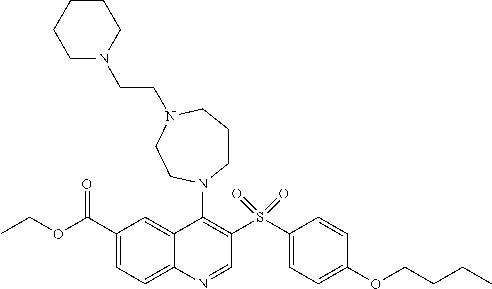 [SMILES: CCCCOc1ccc(S(=O)(=O)c2cnc3ccc(C(=O)OCC)cc3c2N2CCCN(CCN3CCCCC3)CC2)cc1] |
| 182 | 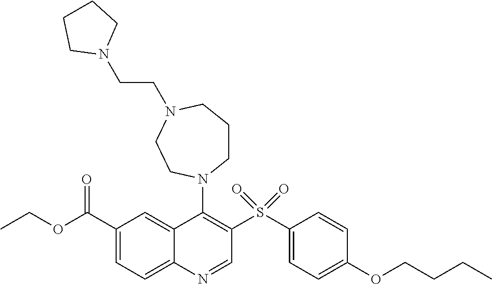 [SMILES: CCCCOc1ccc(S(=O)(=O)c2cnc3ccc(C(=O)OCC)cc3c2N2CCCN(CCN3CCCC3)CC2)cc1] |
| 183 | 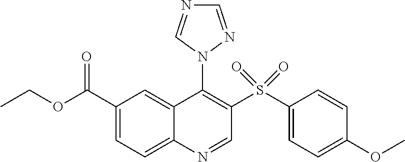 [SMILES: CCOC(=O)c1ccc2ncc(S(=O)(=O)c3ccc(OC)cc3)c(-n3cncn3)c2c1] |
| 184 | 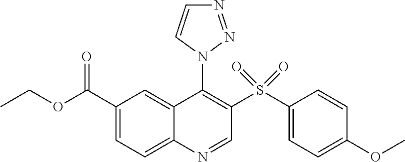 [SMILES: CCOC(=O)c1ccc2ncc(S(=O)(=O)c3ccc(OC)cc3)c(-n3ccnn3)c2c1] |
| 185 | 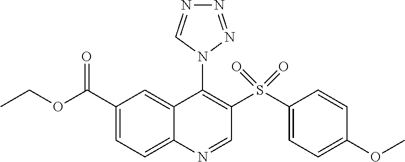 [SMILES: CCOC(=O)c1ccc2ncc(S(=O)(=O)c3ccc(OC)cc3)c(-n3cnnn3)c2c1] |
| 186 | 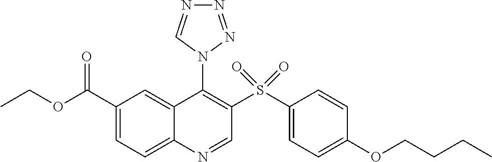 [SMILES: CCCCOc1ccc(S(=O)(=O)c2cnc3ccc(C(=O)OCC)cc3c2-n2cnnn2)cc1] |
| 187 | 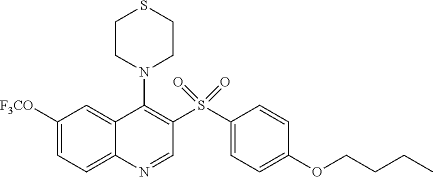 [SMILES: CCCCOc1ccc(S(=O)(=O)c2cnc3ccc(OC(F)(F)F)cc3c2N2CCSCC2)cc1] |
| 188 | 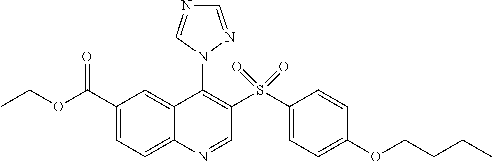 [SMILES: CCCCOc1ccc(S(=O)(=O)c2cnc3ccc(C(=O)OCC)cc3c2-n2cncn2)cc1] |
| 189 | 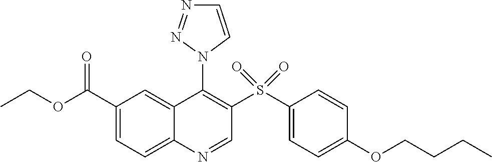 [SMILES: CCCCOc1ccc(S(=O)(=O)c2cnc3ccc(C(=O)OCC)cc3c2-n2ccnn2)cc1] |
| 190 | 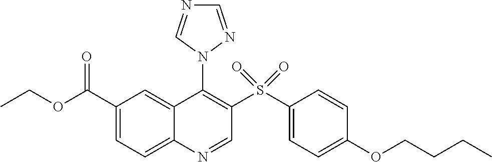 [SMILES: CCCCOc1ccc(S(=O)(=O)c2cnc3ccc(C(=O)OCC)cc3c2-n2cncn2)cc1] |
| 191 | 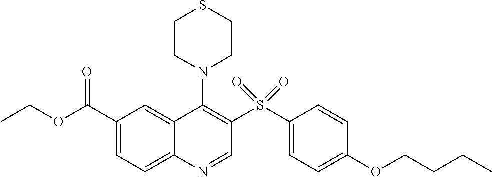 [SMILES: CCCCOc1ccc(S(=O)(=O)c2cnc3ccc(C(=O)OCC)cc3c2N2CCSCC2)cc1] |
| 192 | 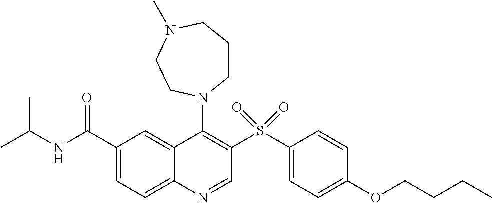 [SMILES: CCCCOc1ccc(S(=O)(=O)c2cnc3ccc(C(=O)NC(C)C)cc3c2N2CCCN(C)CC2)cc1] |
| 193 | 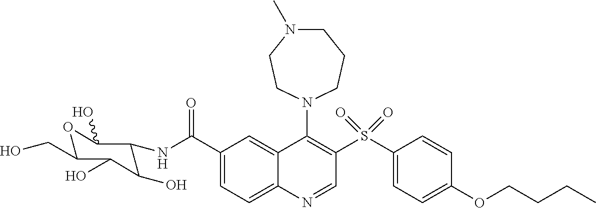 [SMILES: CCCCOc1ccc(S(=O)(=O)c2cnc3ccc(C(=O)NC4C(O)OC(CO)C(O)C4O)cc3c2N2CCCN(C)CC2)cc1] |
| 194 | 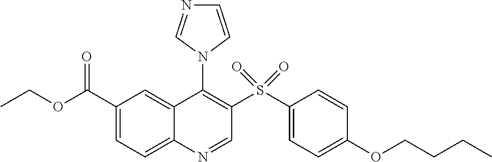 [SMILES: CCCCOc1ccc(S(=O)(=O)c2cnc3ccc(C(=O)OCC)cc3c2-n2ccnc2)cc1] |
| 195 | 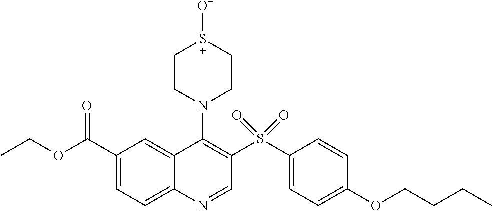 [SMILES: CCCCOc1ccc(S(=O)(=O)c2cnc3ccc(C(=O)OCC)cc3c2N2CC[S+]([O-])CC2)cc1] |
| 196 | 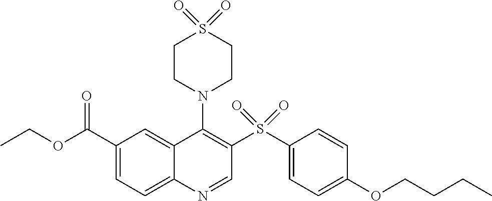 [SMILES: CCCCOc1ccc(S(=O)(=O)c2cnc3ccc(C(=O)OCC)cc3c2N2CCS(=O)(=O)CC2)cc1] |
| 197 | 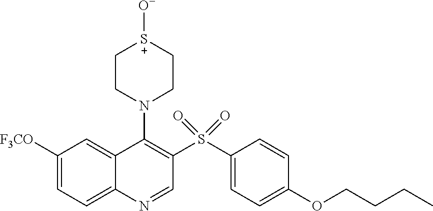 [SMILES: CCCCOc1ccc(S(=O)(=O)c2cnc3ccc(OC(F)(F)F)cc3c2N2CC[S+]([O-])CC2)cc1] |
| 198 | 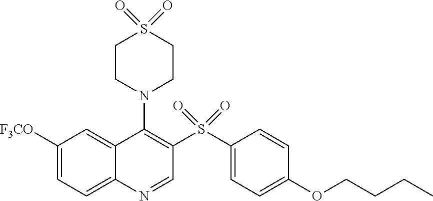 [SMILES: CCCCOc1ccc(S(=O)(=O)c2cnc3ccc(OC(F)(F)F)cc3c2N2CCS(=O)(=O)CC2)cc1] |
| 199 | 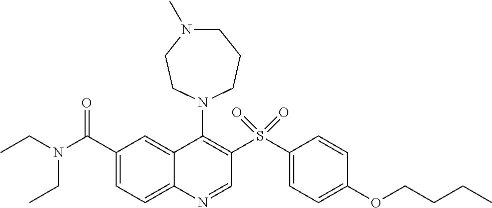 [SMILES: CCCCOc1ccc(S(=O)(=O)c2cnc3ccc(C(=O)N(CC)CC)cc3c2N2CCCN(C)CC2)cc1] |
| 200 | 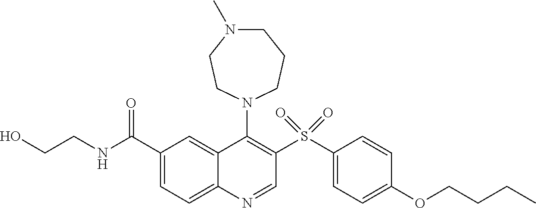 [SMILES: CCCCOc1ccc(S(=O)(=O)c2cnc3ccc(C(=O)NCCO)cc3c2N2CCCN(C)CC2)cc1] |
| 201 | 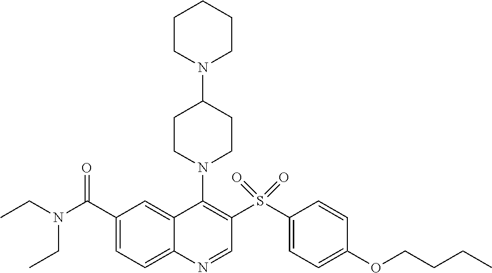 [SMILES: CCCCOc1ccc(S(=O)(=O)c2cnc3ccc(C(=O)N(CC)CC)cc3c2N2CCC(N3CCCCC3)CC2)cc1] |
| 202 | 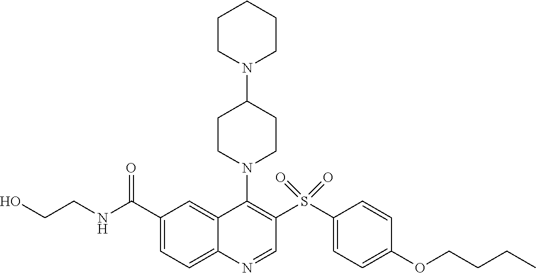 [SMILES: CCCCOc1ccc(S(=O)(=O)c2cnc3ccc(C(=O)NCCO)cc3c2N2CCC(N3CCCCC3)CC2)cc1] |
| 203 | 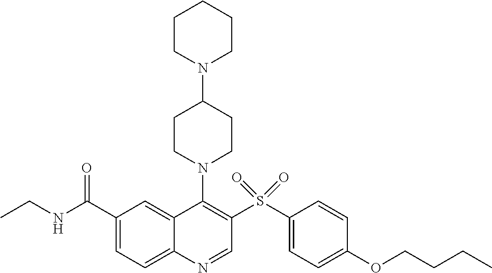 [SMILES: CCCCOc1ccc(S(=O)(=O)c2cnc3ccc(C(=O)NCC)cc3c2N2CCC(N3CCCCC3)CC2)cc1] |
| 204 | 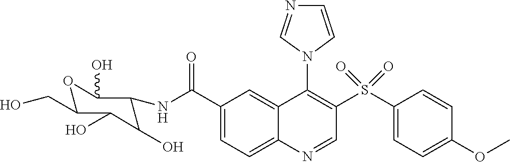 [SMILES: COc1ccc(S(=O)(=O)c2cnc3ccc(C(=O)NC4C(O)OC(CO)C(O)C4O)cc3c2-n2ccnc2)cc1] |
| 205 | 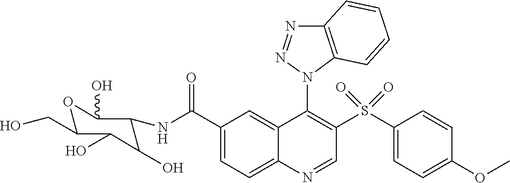 [SMILES: COc1ccc(S(=O)(=O)c2cnc3ccc(C(=O)NC4C(O)OC(CO)C(O)C4O)cc3c2-n2nnc3ccccc32)cc1] |
| 206 | 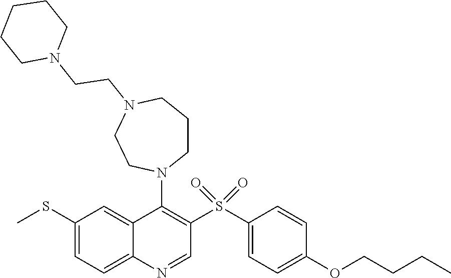 [SMILES: CCCCOc1ccc(S(=O)(=O)c2cnc3ccc(SC)cc3c2N2CCCN(CCN3CCCCC3)CC2)cc1] |
| 207 | 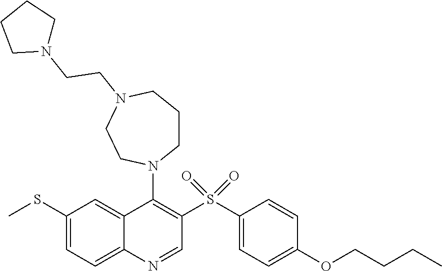 [SMILES: CCCCOc1ccc(S(=O)(=O)c2cnc3ccc(SC)cc3c2N2CCCN(CCN3CCCC3)CC2)cc1] |
| 208 | 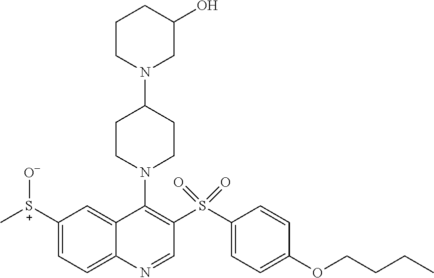 [SMILES: CCCCOc1ccc(S(=O)(=O)c2cnc3ccc([S+](C)[O-])cc3c2N2CCC(N3CCCC(O)C3)CC2)cc1] |
| 209 | 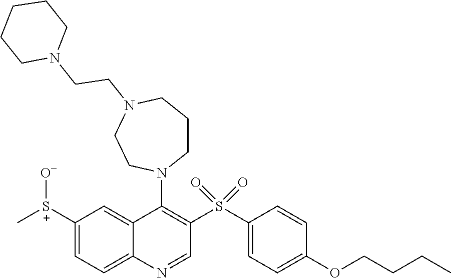 [SMILES: CCCCOc1ccc(S(=O)(=O)c2cnc3ccc([S+](C)[O-])cc3c2N2CCCN(CCN3CCCCC3)CC2)cc1] |
| 210 | 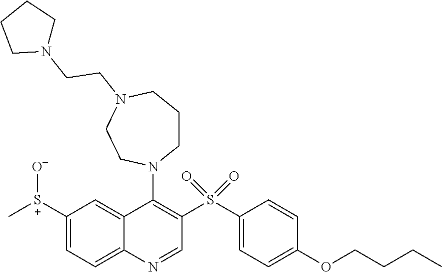 [SMILES: CCCCOc1ccc(S(=O)(=O)c2cnc3ccc([S+](C)[O-])cc3c2N2CCCN(CCN3CCCC3)CC2)cc1] |
| 211 | 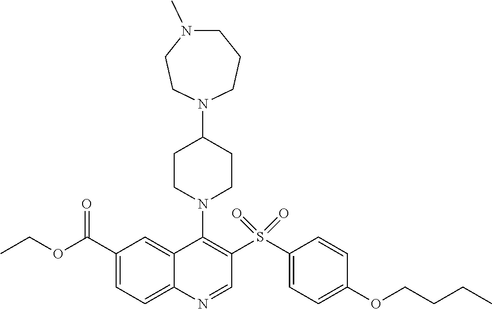 [SMILES: CCCCOc1ccc(S(=O)(=O)c2cnc3ccc(C(=O)OCC)cc3c2N2CCC(N3CCCN(C)CC3)CC2)cc1] |
| 212 | 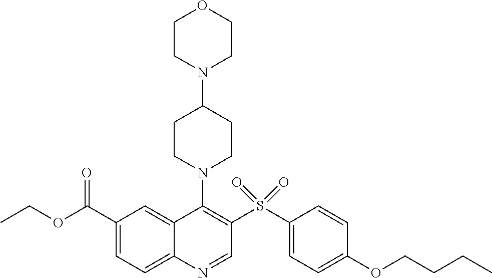 [SMILES: CCCCOc1ccc(S(=O)(=O)c2cnc3ccc(C(=O)OCC)cc3c2N2CCC(N3CCOCC3)CC2)cc1] |
| 213 | 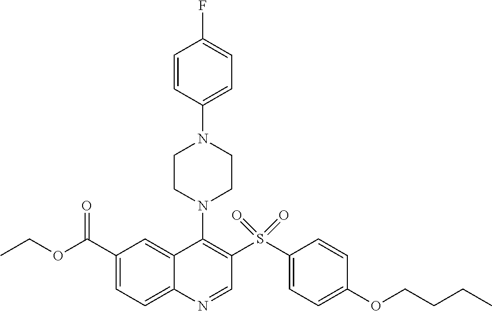 [SMILES: CCCCOc1ccc(S(=O)(=O)c2cnc3ccc(C(=O)OCC)cc3c2N2CCN(c3ccc(F)cc3)CC2)cc1] |
| 214 | 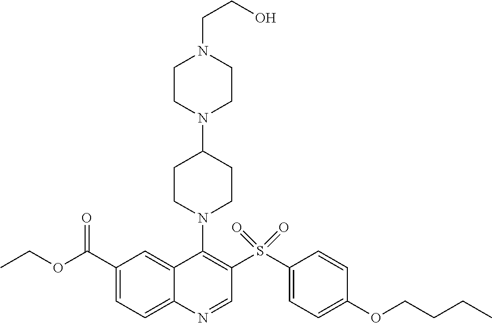 [SMILES: CCCCOc1ccc(S(=O)(=O)c2cnc3ccc(C(=O)OCC)cc3c2N2CCC(N3CCN(CCO)CC3)CC2)cc1] |
| 215 | 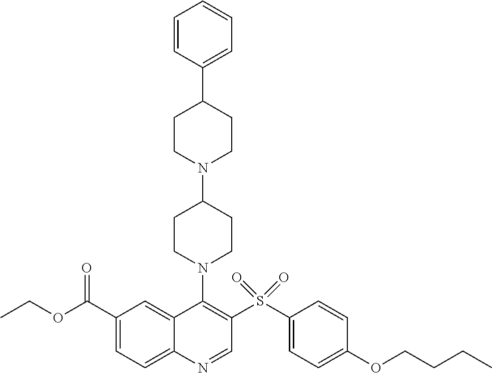 [SMILES: CCCCOc1ccc(S(=O)(=O)c2cnc3ccc(C(=O)OCC)cc3c2N2CCC(N3CCC(c4ccccc4)CC3)CC2)cc1] |
| 216 | 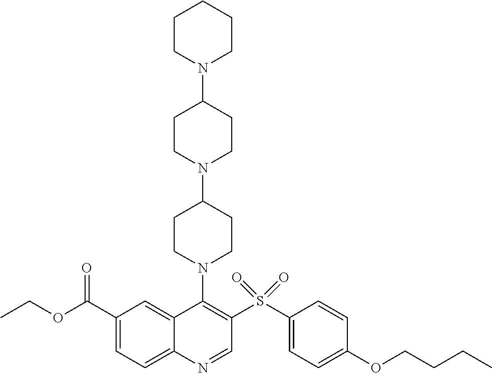 [SMILES: CCCCOc1ccc(S(=O)(=O)c2cnc3ccc(C(=O)OCC)cc3c2N2CCC(N3CCC(N4CCCCC4)CC3)CC2)cc1] |
| 217 | 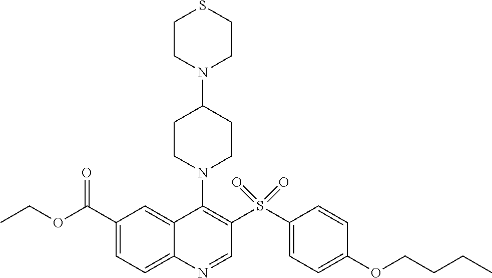 [SMILES: CCCCOc1ccc(S(=O)(=O)c2cnc3ccc(C(=O)OCC)cc3c2N2CCC(N3CCSCC3)CC2)cc1] |
| 218 | 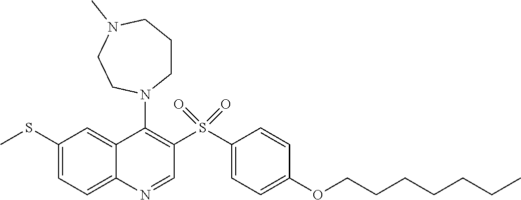 [SMILES: CCCCCCCOc1ccc(S(=O)(=O)c2cnc3ccc(SC)cc3c2N2CCCN(C)CC2)cc1] |
| 219 | 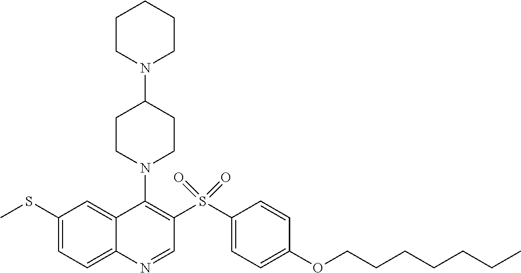 [SMILES: CCCCCCCOc1ccc(S(=O)(=O)c2cnc3ccc(SC)cc3c2N2CCC(N3CCCCC3)CC2)cc1] |
| 220 | 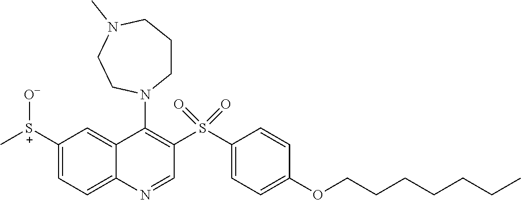 [SMILES: CCCCCCCOc1ccc(S(=O)(=O)c2cnc3ccc([S+](C)[O-])cc3c2N2CCCN(C)CC2)cc1] |
| 221 | 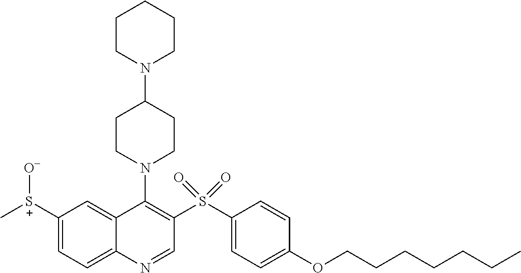 [SMILES: CCCCCCCOc1ccc(S(=O)(=O)c2cnc3ccc([S+](C)[O-])cc3c2N2CCC(N3CCCCC3)CC2)cc1] |
| 222 | 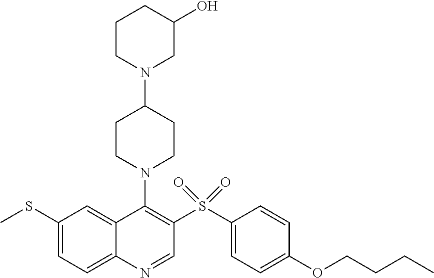 [SMILES: CCCCOc1ccc(S(=O)(=O)c2cnc3ccc(SC)cc3c2N2CCC(N3CCCC(O)C3)CC2)cc1] |
| 223 | 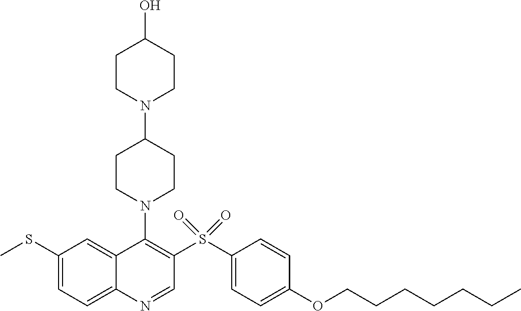 [SMILES: CCCCCCCOc1ccc(S(=O)(=O)c2cnc3ccc(SC)cc3c2N2CCC(N3CCC(O)CC3)CC2)cc1] |
| 224 | 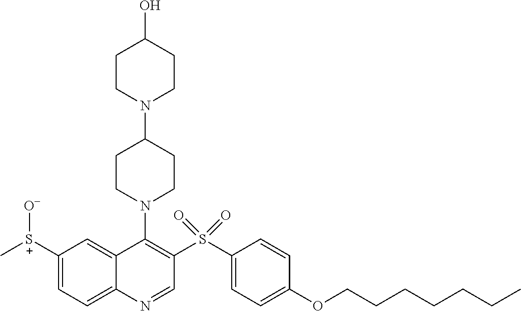 [SMILES: CCCCCCCOc1ccc(S(=O)(=O)c2cnc3ccc([S+](C)[O-])cc3c2N2CCC(N3CCC(O)CC3)CC2)cc1] |
| 225 | 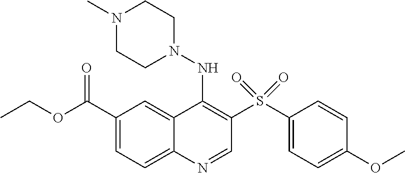 [SMILES: CCOC(=O)c1ccc2ncc(S(=O)(=O)c3ccc(OC)cc3)c(NN3CCN(C)CC3)c2c1] |
| 226 | 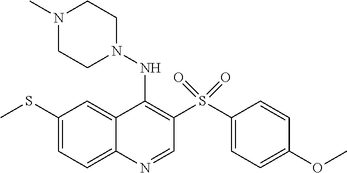 |
| 227 | 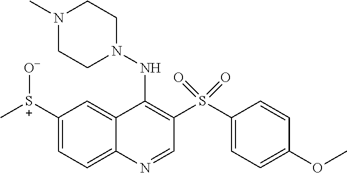 |
| 228 | 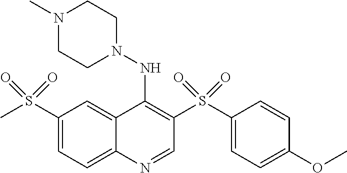 |
| 229 | 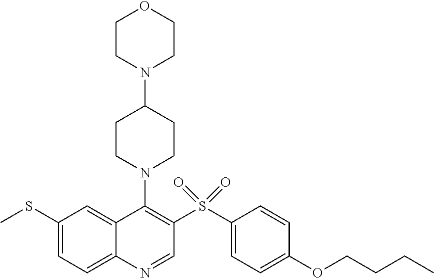 [SMILES: CCCCOc1ccc(S(=O)(=O)c2cnc3ccc(SC)cc3c2N2CCC(N3CCOCC3)CC2)cc1] |
| 230 | 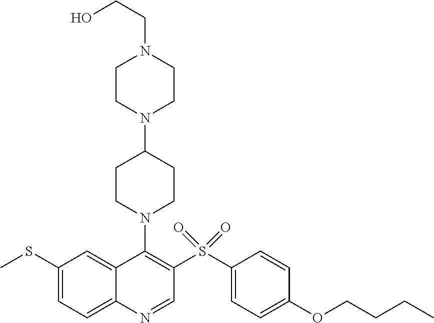 [SMILES: CCCCOc1ccc(S(=O)(=O)c2cnc3ccc(SC)cc3c2N2CCC(N3CCN(CCO)CC3)CC2)cc1] |
| 231 | 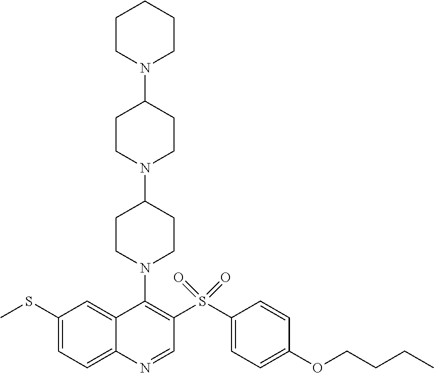 [SMILES: CCCCOc1ccc(S(=O)(=O)c2cnc3ccc(SC)cc3c2N2CCC(N3CCC(N4CCCCC4)CC3)CC2)cc1] |
| 232 | 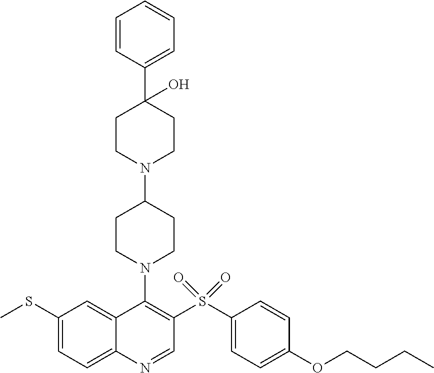 [SMILES: CCCCOc1ccc(S(=O)(=O)c2cnc3ccc(SC)cc3c2N2CCC(N3CCC(O)(c4ccccc4)CC3)CC2)cc1] |
| 233 | 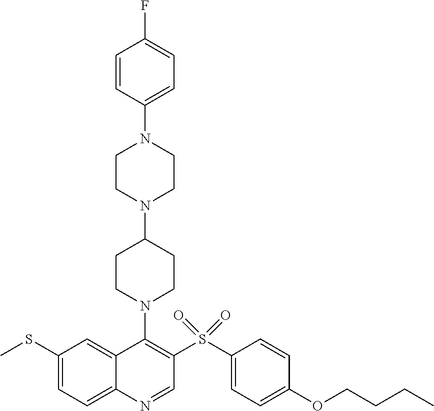 [SMILES: CCCCOc1ccc(S(=O)(=O)c2cnc3ccc(SC)cc3c2N2CCC(N3CCN(c4ccc(F)cc4)CC3)CC2)cc1] |
| 234 | 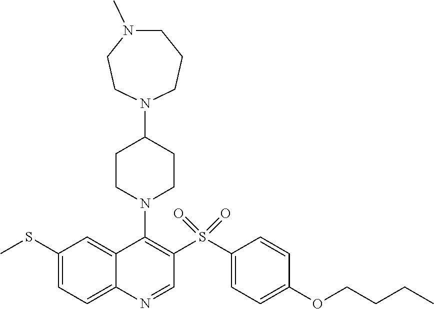 [SMILES: CCCCOc1ccc(S(=O)(=O)c2cnc3ccc(SC)cc3c2N2CCC(N3CCCN(C)CC3)CC2)cc1] |
| 235 | 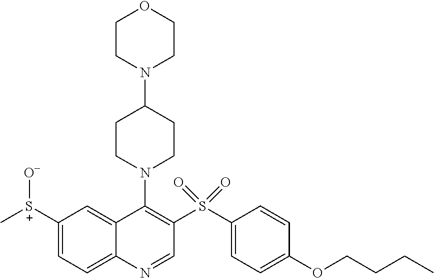 [SMILES: CCCCOc1ccc(S(=O)(=O)c2cnc3ccc([S+](C)[O-])cc3c2N2CCC(N3CCOCC3)CC2)cc1] |
| 236 | 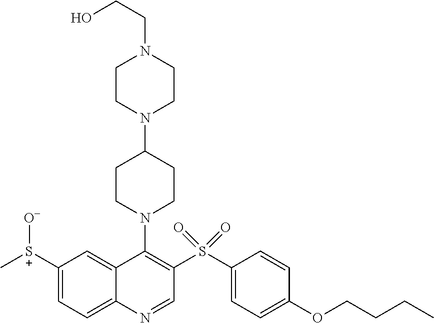 [SMILES: CCCCOc1ccc(S(=O)(=O)c2cnc3ccc([S+](C)[O-])cc3c2N2CCC(N3CCN(CCO)CC3)CC2)cc1] |
| 237 | 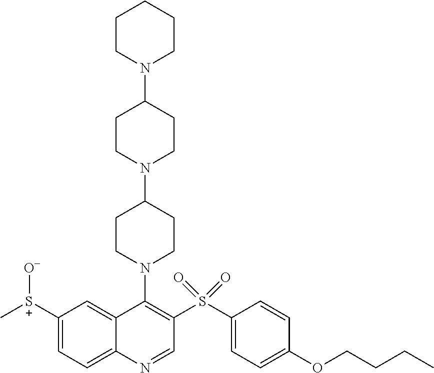 [SMILES: CCCCOc1ccc(S(=O)(=O)c2cnc3ccc([S+](C)[O-])cc3c2N2CCC(N3CCC(N4CCCCC4)CC3)CC2)cc1] |
| 238 | 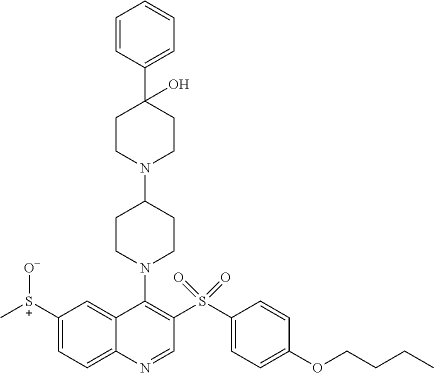 [SMILES: CCCCOc1ccc(S(=O)(=O)c2cnc3ccc([S+](C)[O-])cc3c2N2CCC(N3CCC(O)(c4ccccc4)CC3)CC2)cc1] |
| 239 | 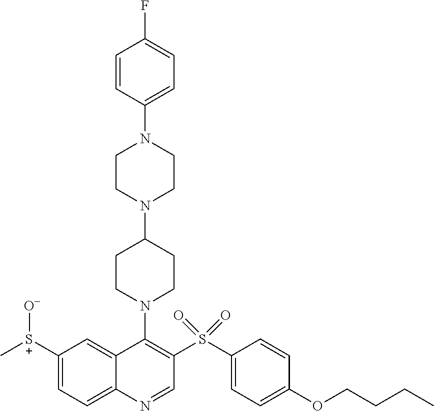 [SMILES: CCCCOc1ccc(S(=O)(=O)c2cnc3ccc([S+](C)[O-])cc3c2N2CCC(N3CCN(c4ccc(F)cc4)CC3)CC2)cc1] |
| 240 | 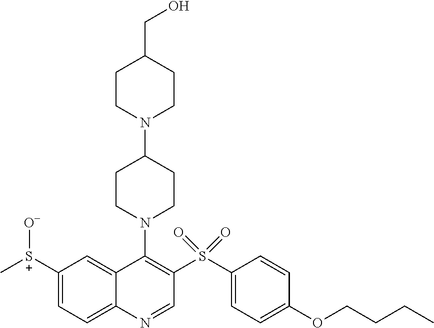 [SMILES: CCCCOc1ccc(S(=O)(=O)c2cnc3ccc([S+](C)[O-])cc3c2N2CCC(N3CCC(CO)CC3)CC2)cc1] |
| 241 | 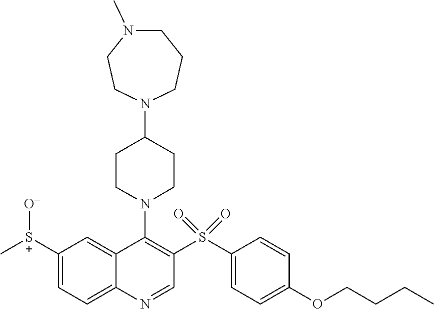 [SMILES: CCCCOc1ccc(S(=O)(=O)c2cnc3ccc([S+](C)[O-])cc3c2N2CCC(N3CCCN(C)CC3)CC2)cc1] |
| 242 | 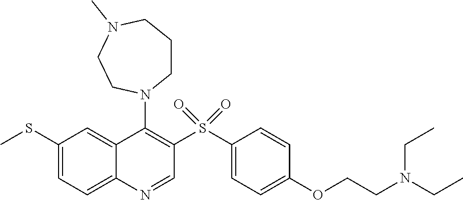 [SMILES: CCN(CC)CCOc1ccc(S(=O)(=O)c2cnc3ccc(SC)cc3c2N2CCCN(C)CC2)cc1] |
| 243 | 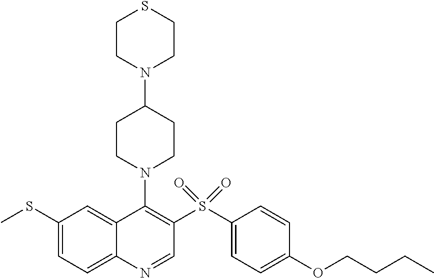 [SMILES: CCCCOc1ccc(S(=O)(=O)c2cnc3ccc(SC)cc3c2N2CCC(N3CCSCC3)CC2)cc1] |
| 244 | 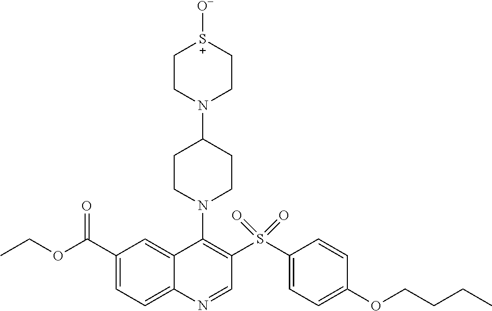 [SMILES: CCCCOc1ccc(S(=O)(=O)c2cnc3ccc(C(=O)OCC)cc3c2N2CCC(N3CC[S+]([O-])CC3)CC2)cc1] |
| 245 | 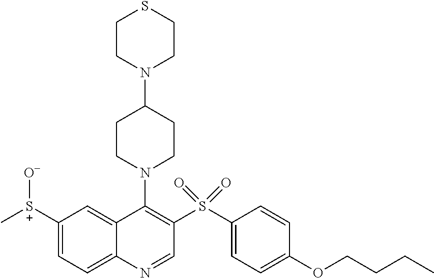 [SMILES: CCCCOc1ccc(S(=O)(=O)c2cnc3ccc([S+](C)[O-])cc3c2N2CCC(N3CCSCC3)CC2)cc1] |
| 246 | 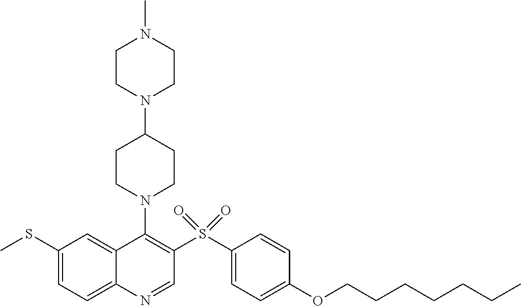 [SMILES: CCCCCCCOc1ccc(S(=O)(=O)c2cnc3ccc(SC)cc3c2N2CCC(N3CCN(C)CC3)CC2)cc1] |
| 247 | 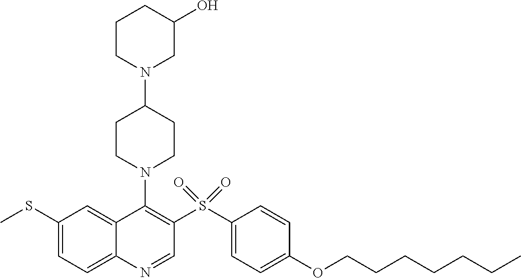 [SMILES: CCCCCCCOc1ccc(S(=O)(=O)c2cnc3ccc(SC)cc3c2N2CCC(N3CCCC(O)C3)CC2)cc1] |
| 248 | 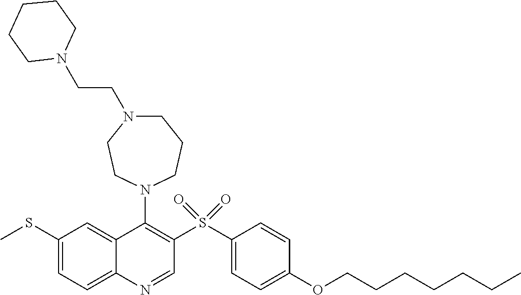 [SMILES: CCCCCCCOc1ccc(S(=O)(=O)c2cnc3ccc(SC)cc3c2N2CCCN(CCN3CCCCC3)CC2)cc1] |
| 249 | 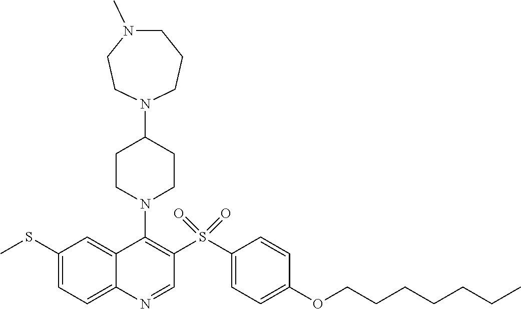 [SMILES: CCCCCCCOc1ccc(S(=O)(=O)c2cnc3ccc(SC)cc3c2N2CCC(N3CCCN(C)CC3)CC2)cc1] |
| 250 | 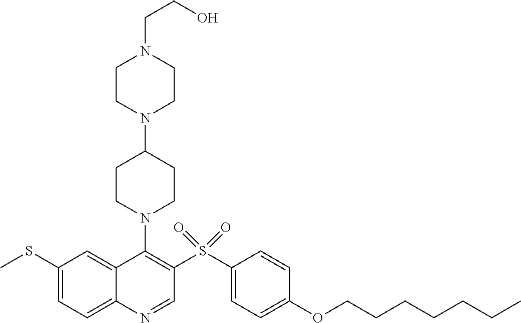 [SMILES: CCCCCCCOc1ccc(S(=O)(=O)c2cnc3ccc(SC)cc3c2N2CCC(N3CCN(CCO)CC3)CC2)cc1] |
| 251 | 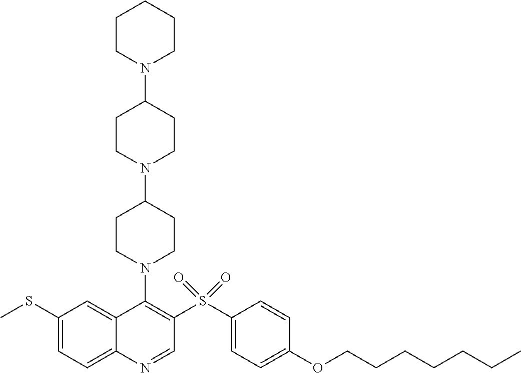 [SMILES: CCCCCCCOc1ccc(S(=O)(=O)c2cnc3ccc(SC)cc3c2N2CCC(N3CCC(N4CCCCC4)CC3)CC2)cc1] |
| 252 | 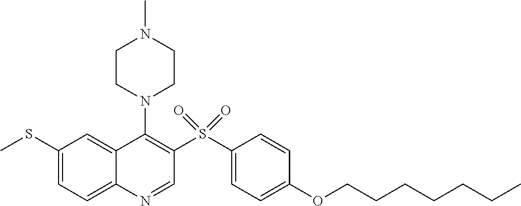 [SMILES: CCCCCCCOc1ccc(S(=O)(=O)c2cnc3ccc(SC)cc3c2N2CCN(C)CC2)cc1] |
| 253 | 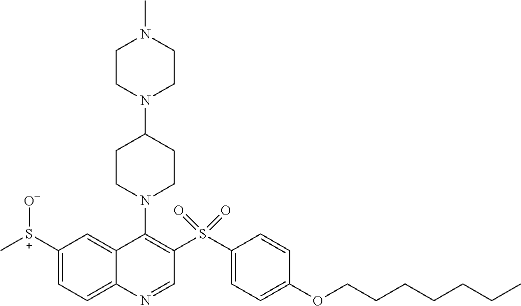 [SMILES: CCCCCCCOc1ccc(S(=O)(=O)c2cnc3ccc([S+](C)[O-])cc3c2N2CCC(N3CCN(C)CC3)CC2)cc1] |
| 254 | 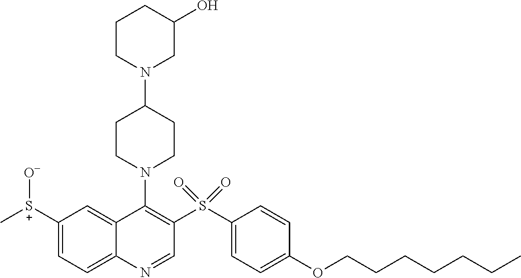 [SMILES: CCCCCCCOc1ccc(S(=O)(=O)c2cnc3ccc([S+](C)[O-])cc3c2N2CCC(N3CCCC(O)C3)CC2)cc1] |
| 255 | 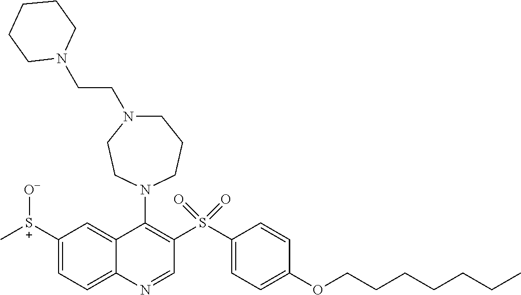 [SMILES: CCCCCCCOc1ccc(S(=O)(=O)c2cnc3ccc([S+](C)[O-])cc3c2N2CCCN(CCN3CCCCC3)CC2)cc1] |
| 256 | 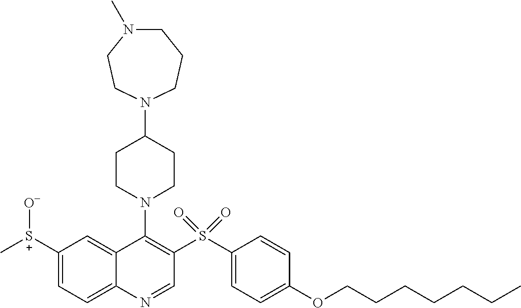 [SMILES: CCCCCCCOc1ccc(S(=O)(=O)c2cnc3ccc([S+](C)[O-])cc3c2N2CCC(N3CCCN(C)CC3)CC2)cc1] |
| 257 | 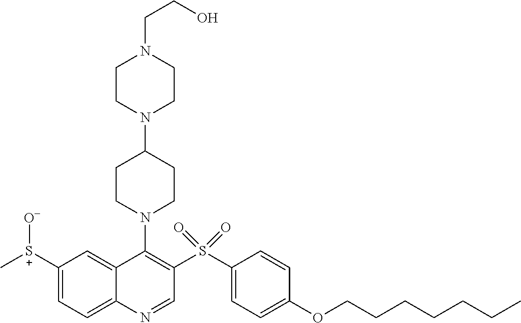 [SMILES: CCCCCCCOc1ccc(S(=O)(=O)c2cnc3ccc([S+](C)[O-])cc3c2N2CCC(N3CCN(CCO)CC3)CC2)cc1] |
| 258 | 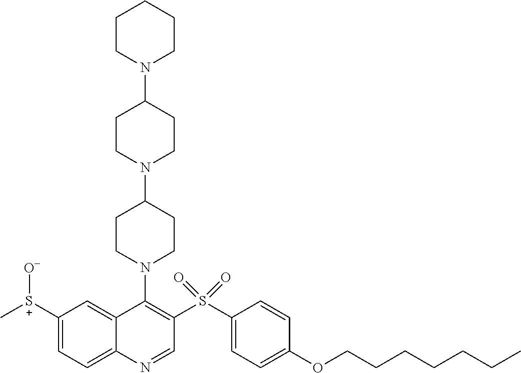 [SMILES: CCCCCCCOc1ccc(S(=O)(=O)c2cnc3ccc([S+](C)[O-])cc3c2N2CCC(N3CCC(N4CCCCC4)CC3)CC2)cc1] |
| 259 | 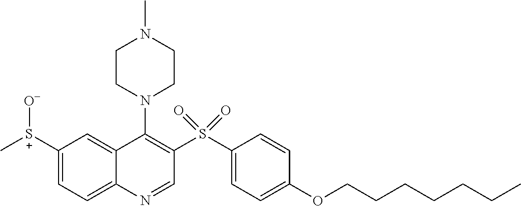 [SMILES: CCCCCCCOc1ccc(S(=O)(=O)c2cnc3ccc([S+](C)[O-])cc3c2N2CCN(C)CC2)cc1] |
| 260 | 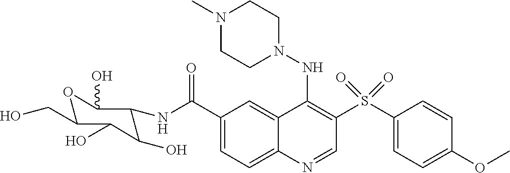 [SMILES: COc1ccc(S(=O)(=O)c2cnc3ccc(C(=O)NC4C(O)OC(CO)C(O)C4O)cc3c2NN2CCN(C)CC2)cc1] |
| 262 | 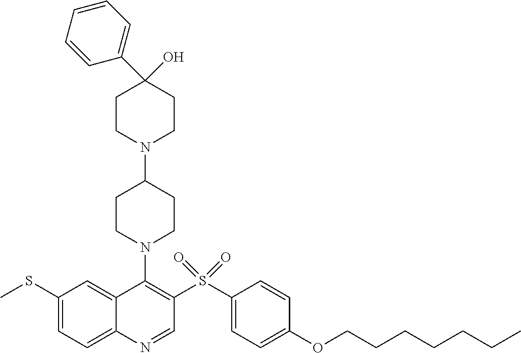 [SMILES: CCCCCCCOc1ccc(S(=O)(=O)c2cnc3ccc(SC)cc3c2N2CCC(N3CCC(O)(c4ccccc4)CC3)CC2)cc1] |
| 263 | 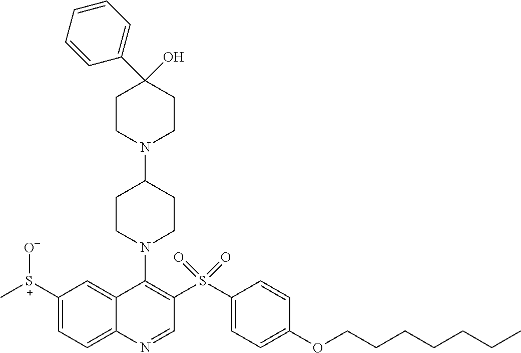 [SMILES: CCCCCCCOc1ccc(S(=O)(=O)c2cnc3ccc([S+](C)[O-])cc3c2N2CCC(N3CCC(O)(c4ccccc4)CC3)CC2)cc1] |
| 264 | 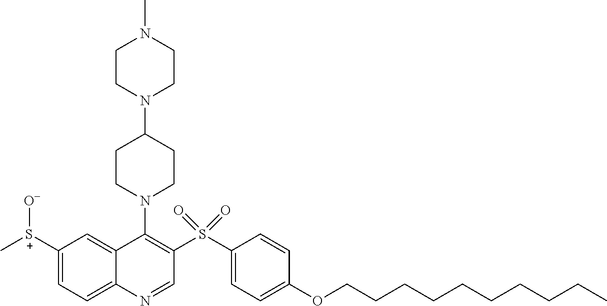 [SMILES: CCCCCCCCCCOc1ccc(S(=O)(=O)c2cnc3ccc([S+](C)[O-])cc3c2N2CCC(N3CCN(C)CC3)CC2)cc1] |
| 265 | 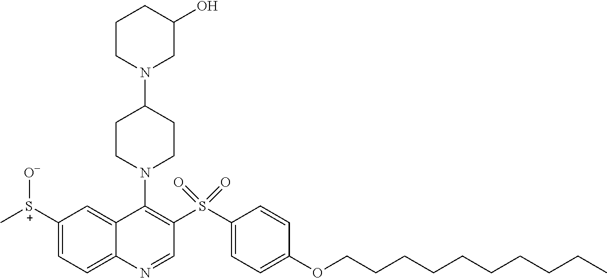 [SMILES: CCCCCCCCCCOc1ccc(S(=O)(=O)c2cnc3ccc([S+](C)[O-])cc3c2N2CCC(N3CCCC(O)C3)CC2)cc1] |
| 266 | 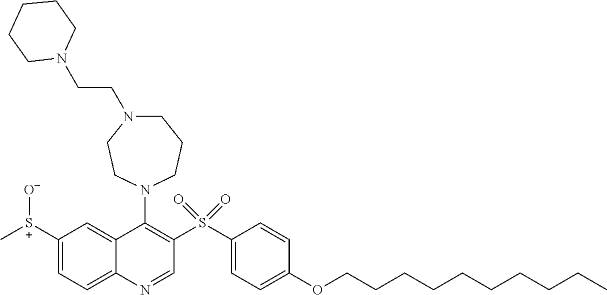 [SMILES: CCCCCCCCCCOc1ccc(S(=O)(=O)c2cnc3ccc([S+](C)[O-])cc3c2N2CCCN(CCN3CCCCC3)CC2)cc1] |
| 267 | 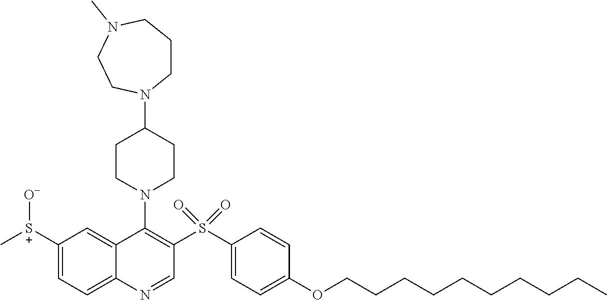 [SMILES: CCCCCCCCCCOc1ccc(S(=O)(=O)c2cnc3ccc([S+](C)[O-])cc3c2N2CCC(N3CCCN(C)CC3)CC2)cc1] |
| 268 | 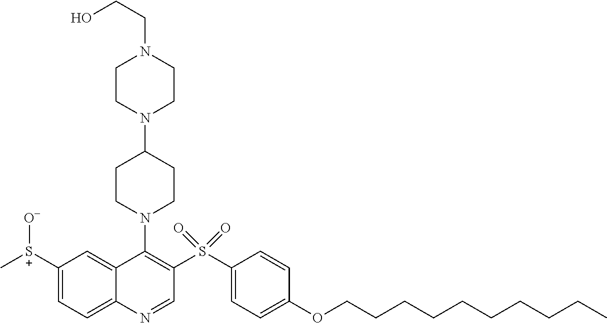 [SMILES: CCCCCCCCCCOc1ccc(S(=O)(=O)c2cnc3ccc([S+](C)[O-])cc3c2N2CCC(N3CCN(CCO)CC3)CC2)cc1] |
| 269 | 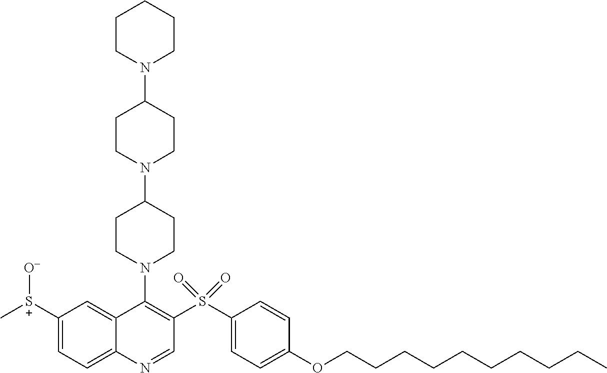 [SMILES: CCCCCCCCCCOc1ccc(S(=O)(=O)c2cnc3ccc([S+](C)[O-])cc3c2N2CCC(N3CCC(N4CCCCC4)CC3)CC2)cc1] |
| 270 | 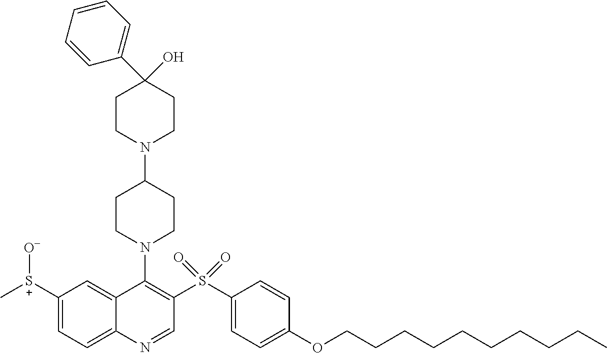 [SMILES: CCCCCCCCCCOc1ccc(S(=O)(=O)c2cnc3ccc([S+](C)[O-])cc3c2N2CCC(N3CCC(O)(c4ccccc4)CC3)CC2)cc1] |
| 271 | 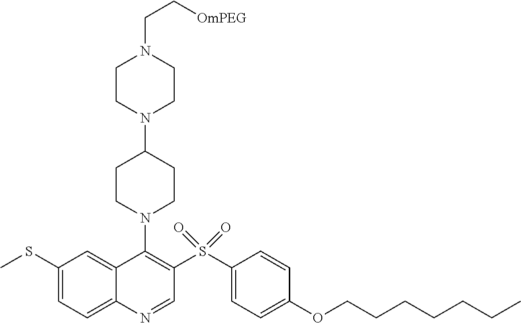 |
| 272 | 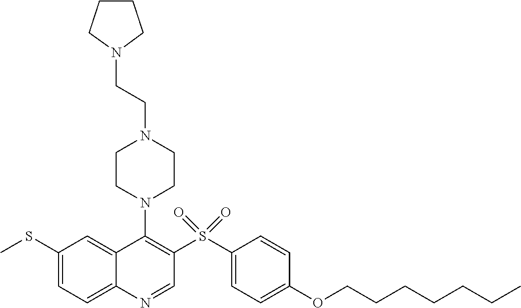 [SMILES: CCCCCCCOc1ccc(S(=O)(=O)c2cnc3ccc(SC)cc3c2N2CCN(CCN3CCCC3)CC2)cc1] |
| 273 | 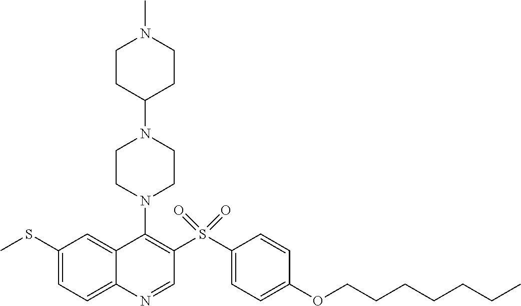 [SMILES: CCCCCCCOc1ccc(S(=O)(=O)c2cnc3ccc(SC)cc3c2N2CCN(C3CCN(C)CC3)CC2)cc1] |
| 274 | 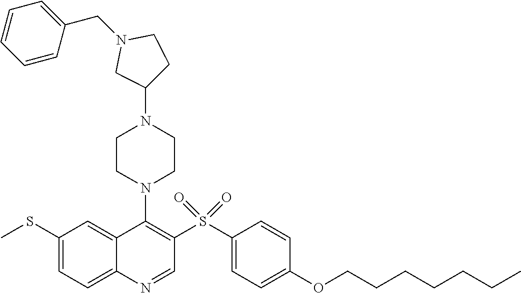 [SMILES: CCCCCCCOc1ccc(S(=O)(=O)c2cnc3ccc(SC)cc3c2N2CCN(C3CCN(Cc4ccccc4)C3)CC2)cc1] |
| 275 | 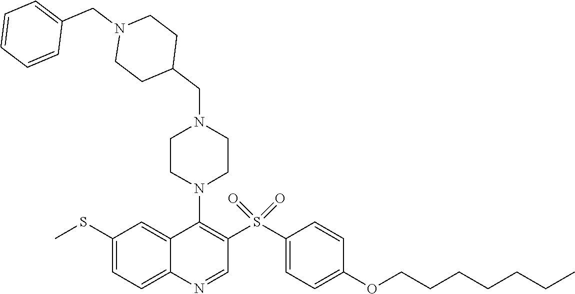 [SMILES: CCCCCCCOc1ccc(S(=O)(=O)c2cnc3ccc(SC)cc3c2N2CCN(CC3CCN(Cc4ccccc4)CC3)CC2)cc1] |
| 276 | 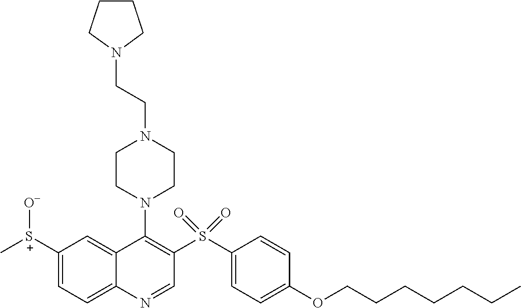 [SMILES: CCCCCCCOc1ccc(S(=O)(=O)c2cnc3ccc([S+](C)[O-])cc3c2N2CCN(CCN3CCCC3)CC2)cc1] |
| 277 | 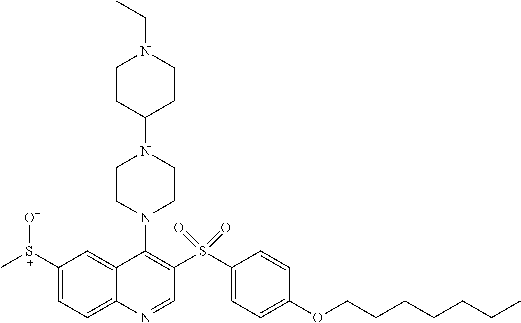 [SMILES: CCCCCCCOc1ccc(S(=O)(=O)c2cnc3ccc([S+](C)[O-])cc3c2N2CCN(C3CCN(CC)CC3)CC2)cc1] |
| 278 | 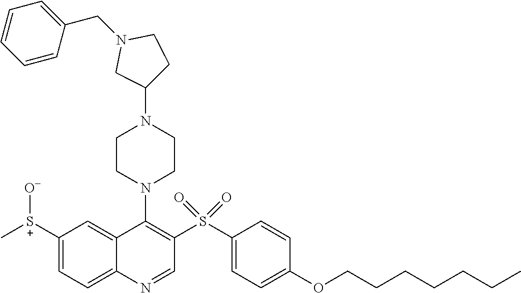 [SMILES: CCCCCCCOc1ccc(S(=O)(=O)c2cnc3ccc([S+](C)[O-])cc3c2N2CCN(C3CCN(Cc4ccccc4)C3)CC2)cc1] |
| 279 | 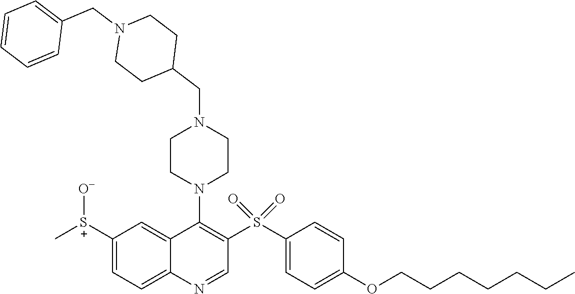 [SMILES: CCCCCCCOc1ccc(S(=O)(=O)c2cnc3ccc([S+](C)[O-])cc3c2N2CCN(CC3CCN(Cc4ccccc4)CC3)CC2)cc1] |
| 280 | 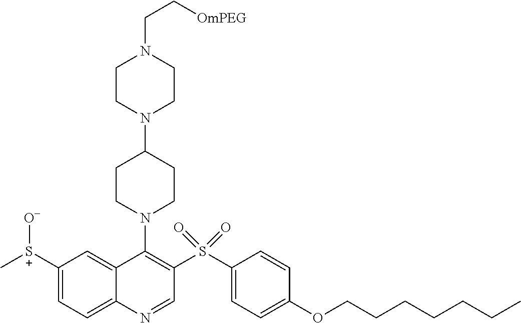 |
| 281 | 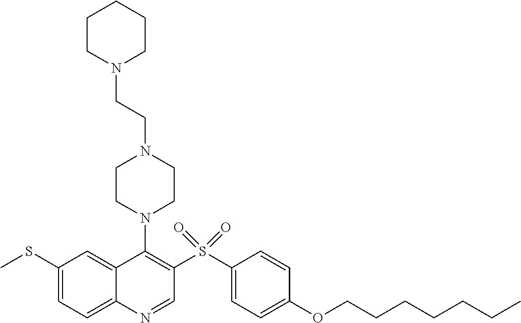 [SMILES: CCCCCCCOc1ccc(S(=O)(=O)c2cnc3ccc(SC)cc3c2N2CCN(CCN3CCCCC3)CC2)cc1] |
| 282 | 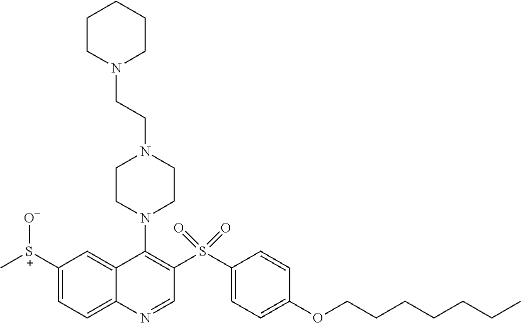 [SMILES: CCCCCCCOc1ccc(S(=O)(=O)c2cnc3ccc([S+](C)[O-])cc3c2N2CCN(CCN3CCCCC3)CC2)cc1] |
| 283 | 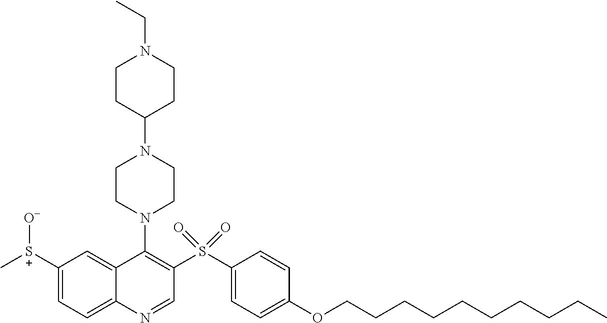 [SMILES: CCCCCCCCCCOc1ccc(S(=O)(=O)c2cnc3ccc([S+](C)[O-])cc3c2N2CCN(C3CCN(CC)CC3)CC2)cc1] |
| 284 | 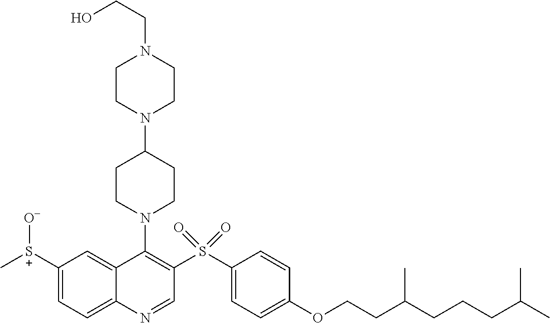 [SMILES: CC(C)CCCC(C)CCOc1ccc(S(=O)(=O)c2cnc3ccc([S+](C)[O-])cc3c2N2CCC(N3CCN(CCO)CC3)CC2)cc1] |
| 285 | 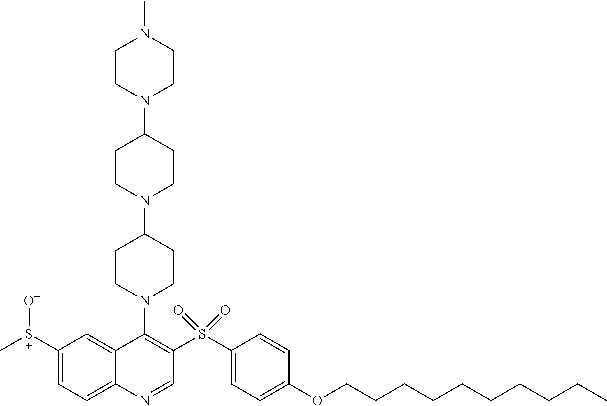 [SMILES: CCCCCCCCCCOc1ccc(S(=O)(=O)c2cnc3ccc([S+](C)[O-])cc3c2N2CCC(N3CCC(N4CCN(C)CC4)CC3)CC2)cc1] |
| 286 | 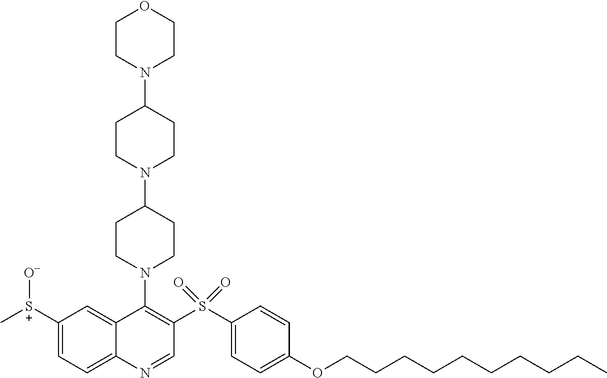 [SMILES: CCCCCCCCCCOc1ccc(S(=O)(=O)c2cnc3ccc([S+](C)[O-])cc3c2N2CCC(N3CCC(N4CCOCC4)CC3)CC2)cc1] |
| 287 | 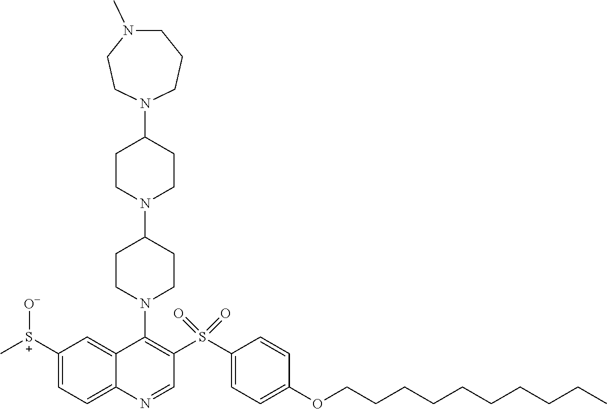 [SMILES: CCCCCCCCCCOc1ccc(S(=O)(=O)c2cnc3ccc([S+](C)[O-])cc3c2N2CCC(N3CCC(N4CCCN(C)CC4)CC3)CC2)cc1] |
| 288 | 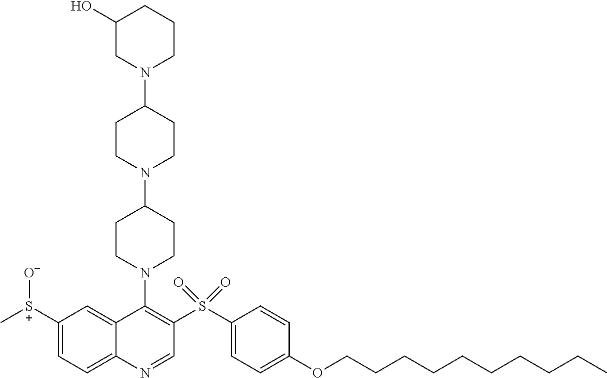 [SMILES: CCCCCCCCCCOc1ccc(S(=O)(=O)c2cnc3ccc([S+](C)[O-])cc3c2N2CCC(N3CCC(N4CCCC(O)C4)CC3)CC2)cc1] |
| 289 | 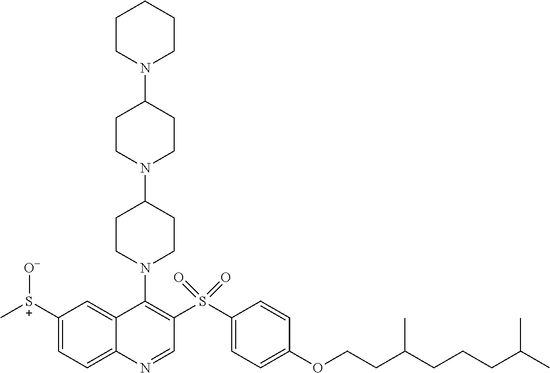 [SMILES: CC(C)CCCC(C)CCOc1ccc(S(=O)(=O)c2cnc3ccc([S+](C)[O-])cc3c2N2CCC(N3CCC(N4CCCCC4)CC3)CC2)cc1] |
| 290 | 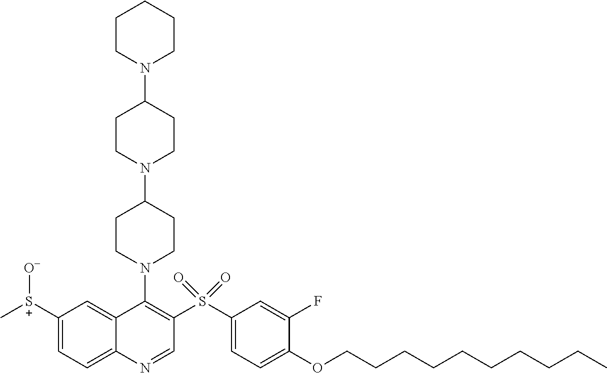 [SMILES: CCCCCCCCCCOc1ccc(S(=O)(=O)c2cnc3ccc([S+](C)[O-])cc3c2N2CCC(N3CCC(N4CCCCC4)CC3)CC2)cc1F] |
| 291 | 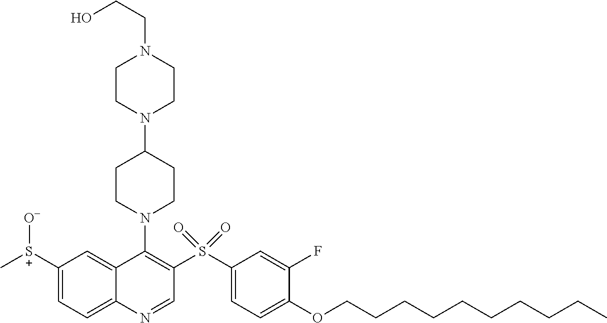 [SMILES: CCCCCCCCCCOc1ccc(S(=O)(=O)c2cnc3ccc([S+](C)[O-])cc3c2N2CCC(N3CCN(CCO)CC3)CC2)cc1F] |
| 292 | 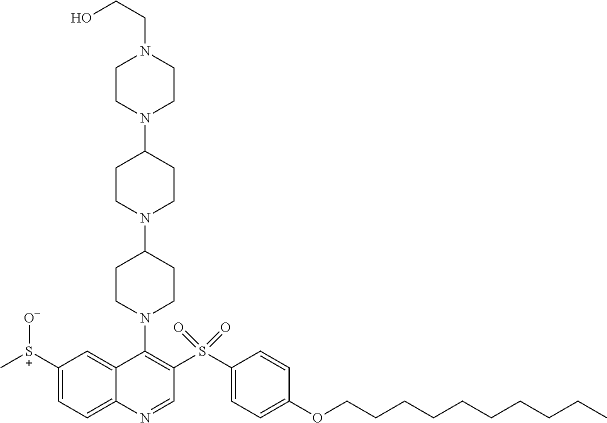 [SMILES: CCCCCCCCCCOc1ccc(S(=O)(=O)c2cnc3ccc([S+](C)[O-])cc3c2N2CCC(N3CCC(N4CCN(CCO)CC4)CC3)CC2)cc1] |
| 293 | 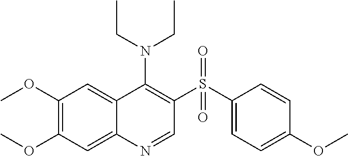 |
| 294 | 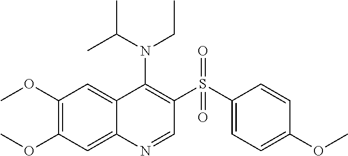 |
| 295 | 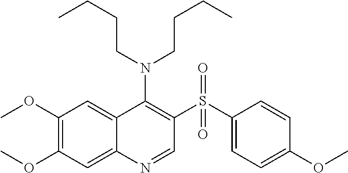 |
| 296 | 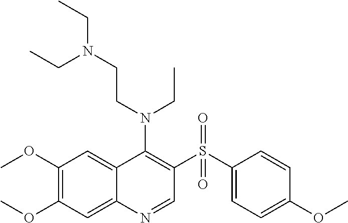 |
| 297 | 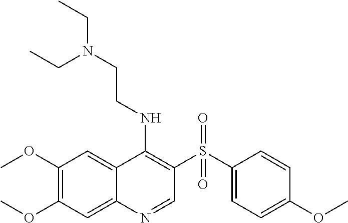 |
| 298 | 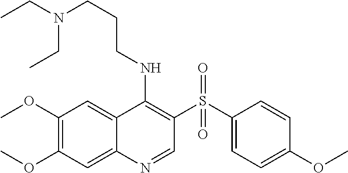 |
| 299 | 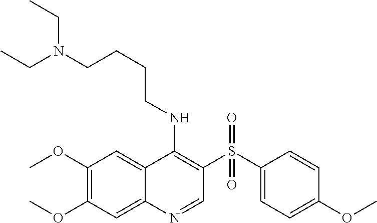 [SMILES: CCN(CC)CCCCNc1c(S(=O)(=O)c2ccc(OC)cc2)cnc2cc(OC)c(OC)cc12] |
| 300 | 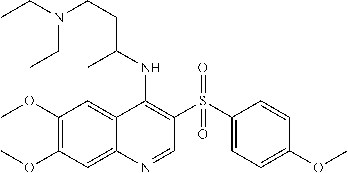 |
| 301 | 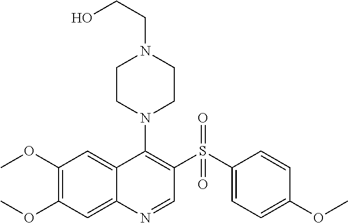 |
| 302 | 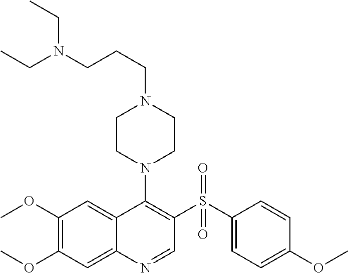 |
| 303 | 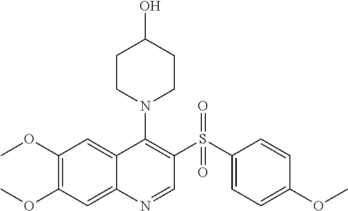 |
| 304 | 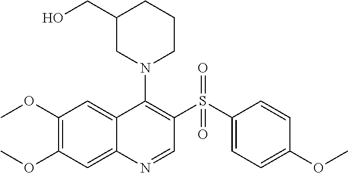 |
| 305 | 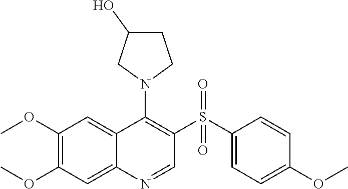 |
| 306 | 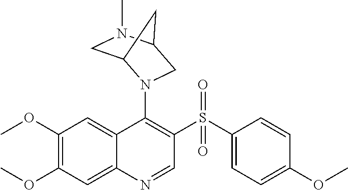 |
| 307 | 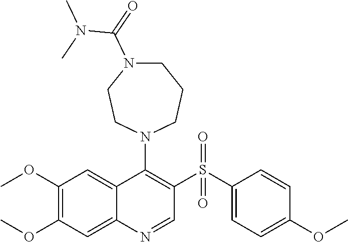 |
| 308 | 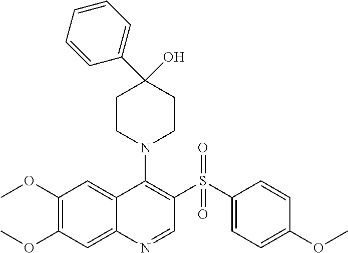 |
| 309 | 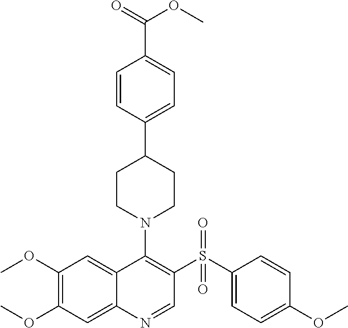 |
| 310 | 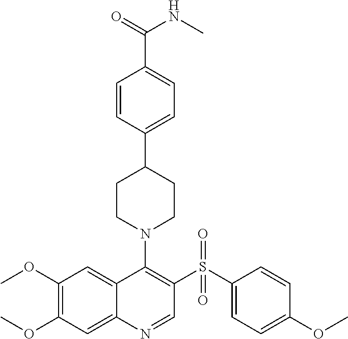 |
| 311 | 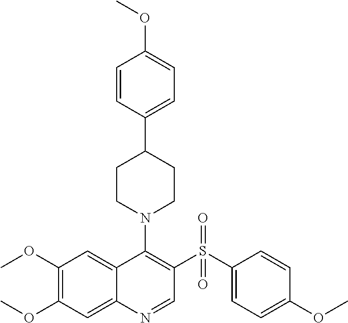 |
| 312 | 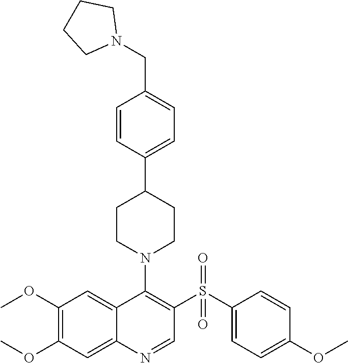 |
| 313 | 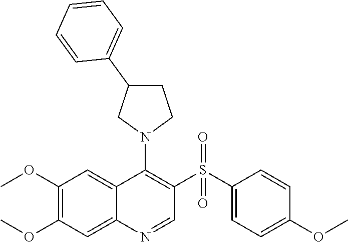 |
| 314 | 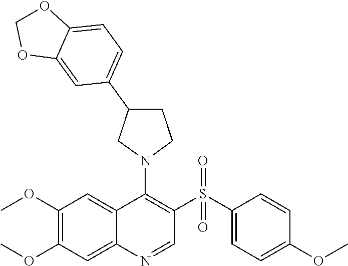 |
| 315 | 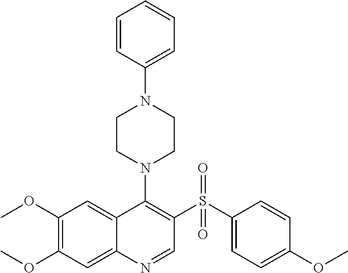 |
| 316 | 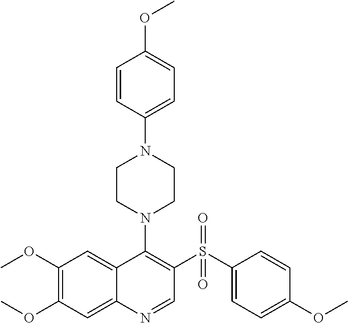 |
| 317 | 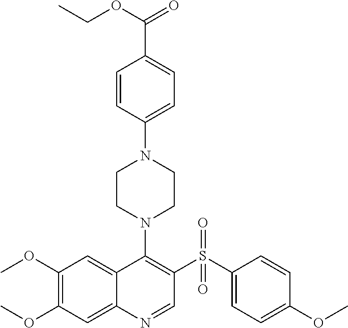 |
| 318 | 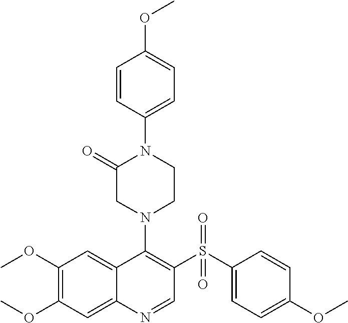 |
| 319 | 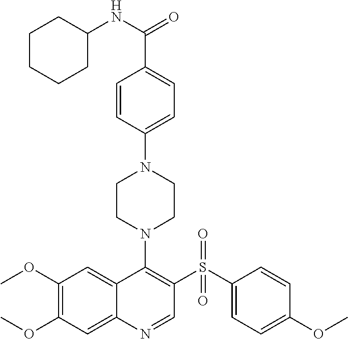 |
| 320 | 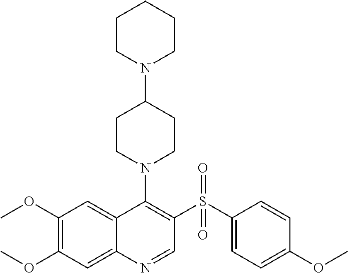 |
| 321 | 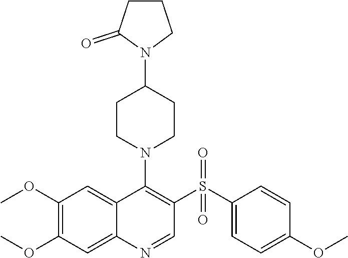 |
| 322 | 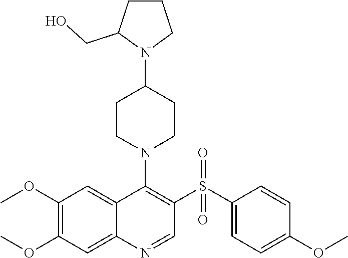 |
| 323 | 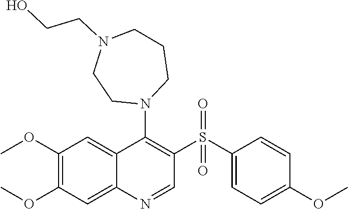 |
| 324 | 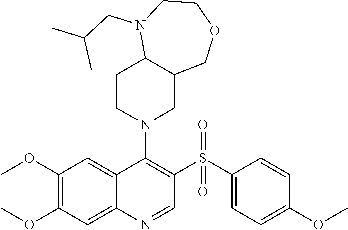 |
| 325 | 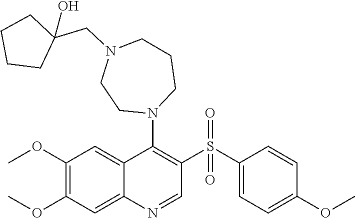 [SMILES: COc1ccc(S(=O)(=O)c2cnc3cc(OC)c(OC)cc3c2N2CCCN(CC3(O)CCCC3)CC2)cc1] |
| 326 | 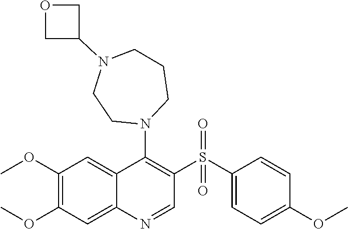 |
| 327 | 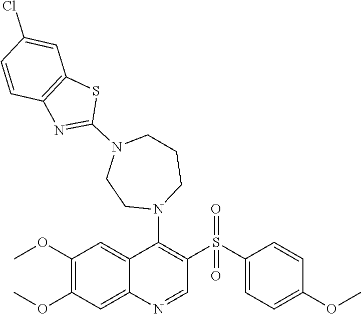 [SMILES: COc1ccc(S(=O)(=O)c2cnc3cc(OC)c(OC)cc3c2N2CCCN(c3nc4ccc(Cl)cc4s3)CC2)cc1] |
| 328 | 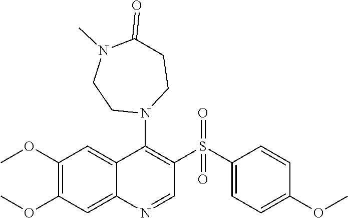 |
| 329 | 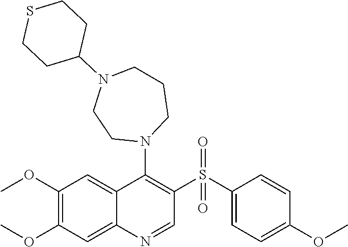 |
| 330 | 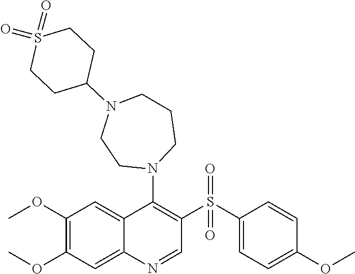 [SMILES: COc1ccc(S(=O)(=O)c2cnc3cc(OC)c(OC)cc3c2N2CCCN(C3CCS(=O)(=O)CC3)CC2)cc1] |
| 331 | 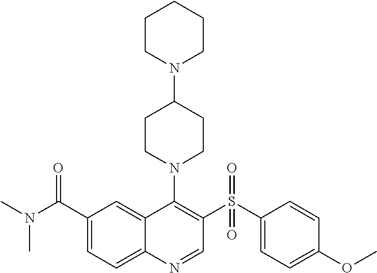 |
| 332 | 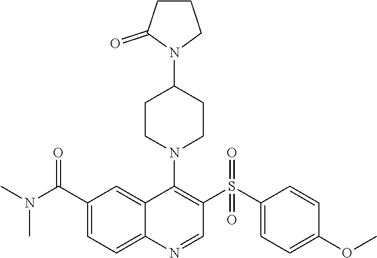 |
| 333 | 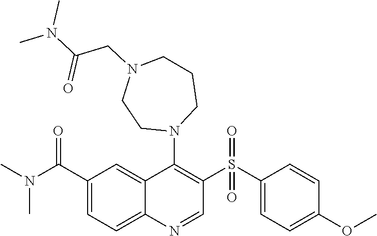 |
| 334 | 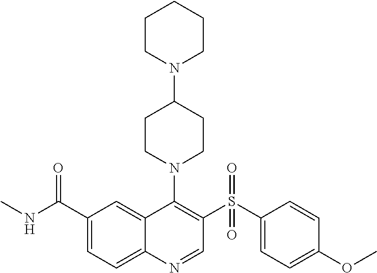 |
| 335 | 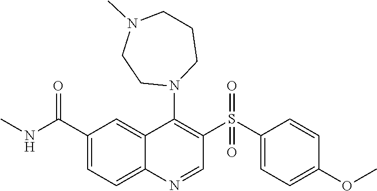 |
| 336 | 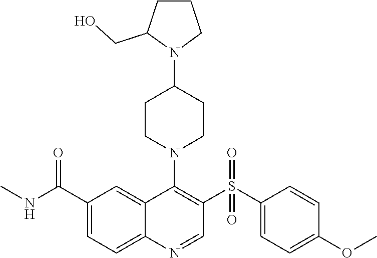 |
| 337 | 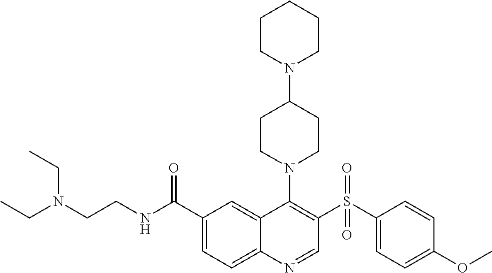 [SMILES: CCN(CC)CCNC(=O)c1ccc2ncc(S(=O)(=O)c3ccc(OC)cc3)c(N3CCC(N4CCCCC4)CC3)c2c1] |
| 338 | 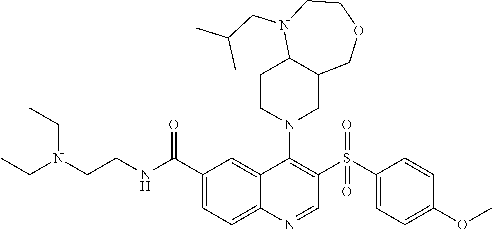 [SMILES: CCN(CC)CCNC(=O)c1ccc2ncc(S(=O)(=O)c3ccc(OC)cc3)c(N3CCC4C(COCCN4CC(C)C)C3)c2c1] |
| 339 | 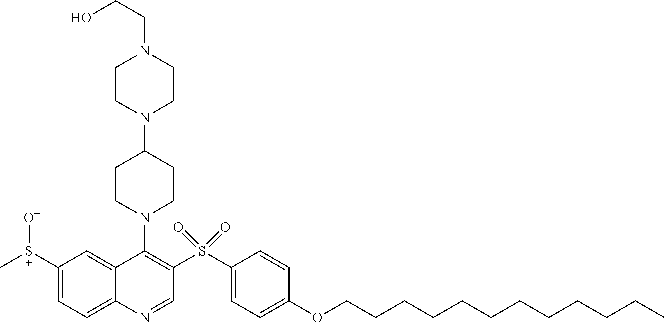 [SMILES: CCCCCCCCCCCCOc1ccc(S(=O)(=O)c2cnc3ccc([S+](C)[O-])cc3c2N2CCC(N3CCN(CCO)CC3)CC2)cc1] |
| 340 | 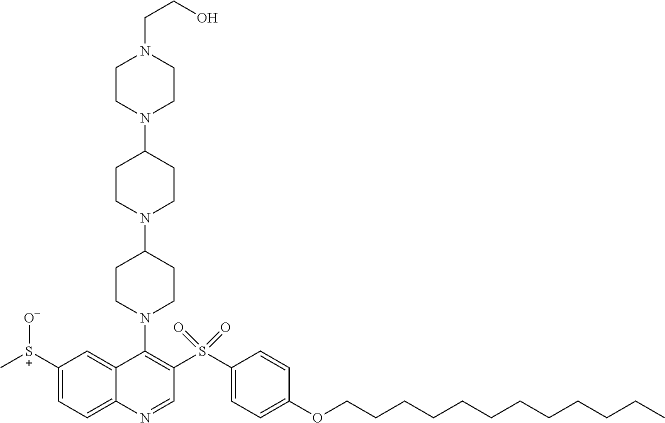 [SMILES: CCCCCCCCCCCCOc1ccc(S(=O)(=O)c2cnc3ccc([S+](C)[O-])cc3c2N2CCC(N3CCC(N4CCN(CCO)CC4)CC3)CC2)cc1] |
| 341 | 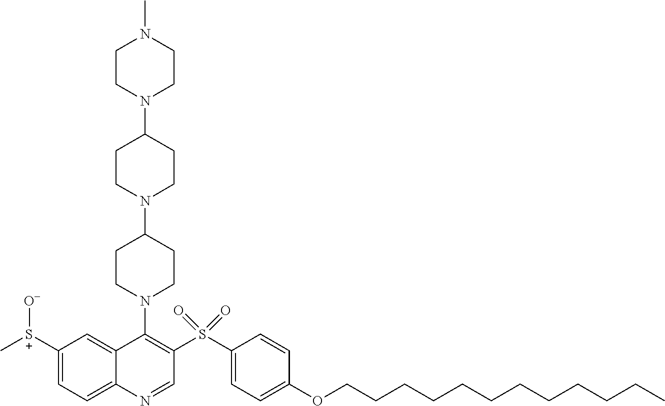 [SMILES: CCCCCCCCCCCCOc1ccc(S(=O)(=O)c2cnc3ccc([S+](C)[O-])cc3c2N2CCC(N3CCC(N4CCN(C)CC4)CC3)CC2)cc1] |
| 342 | 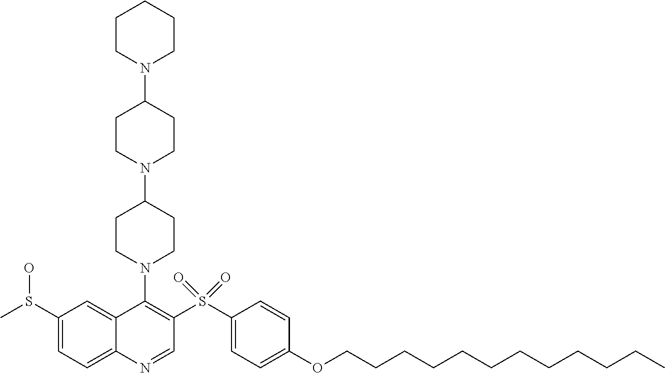 |
| 343 | 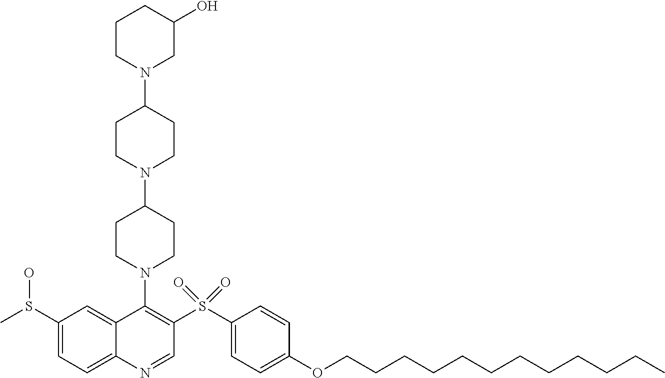 |
| 344 | 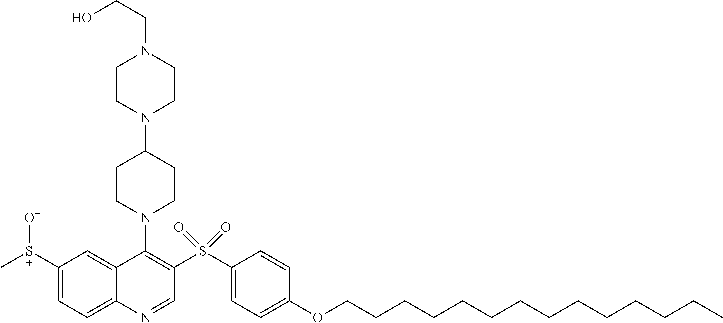 [SMILES: CCCCCCCCCCCCCCOc1ccc(S(=O)(=O)c2cnc3ccc([S+](C)[O-])cc3c2N2CCC(N3CCN(CCO)CC3)CC2)cc1] |
| 345 | 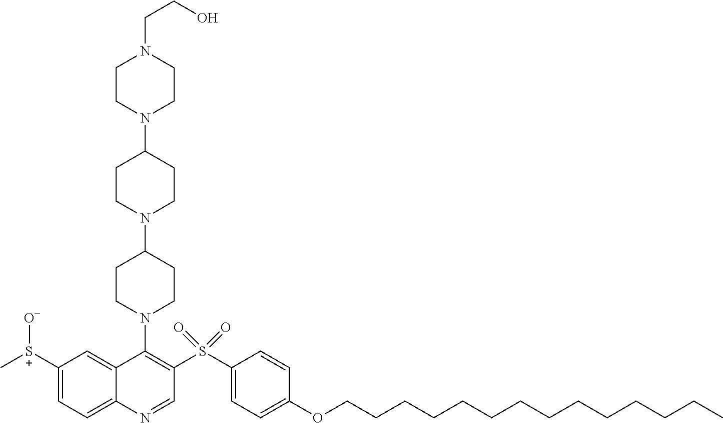 [SMILES: CCCCCCCCCCCCCCOc1ccc(S(=O)(=O)c2cnc3ccc([S+](C)[O-])cc3c2N2CCC(N3CCC(N4CCN(CCO)CC4)CC3)CC2)cc1] |
| 346 | 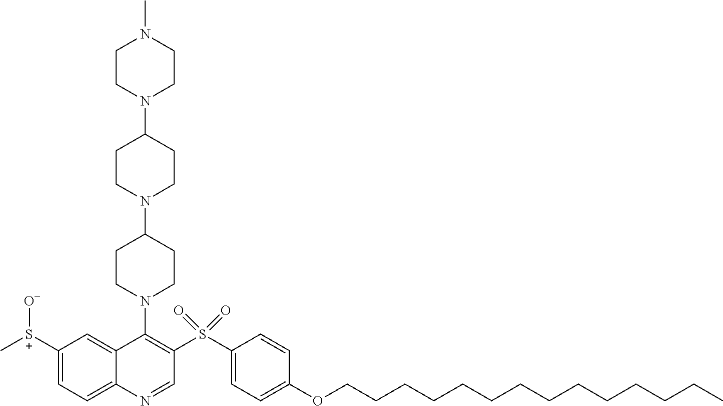 [SMILES: CCCCCCCCCCCCCCOc1ccc(S(=O)(=O)c2cnc3ccc([S+](C)[O-])cc3c2N2CCC(N3CCC(N4CCN(C)CC4)CC3)CC2)cc1] |
| 347 | 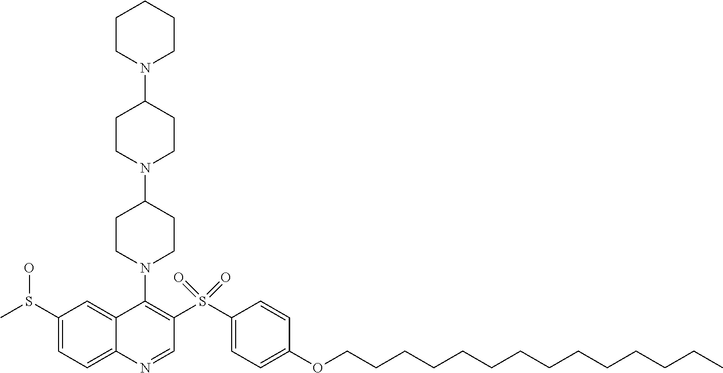 |
| 348 | 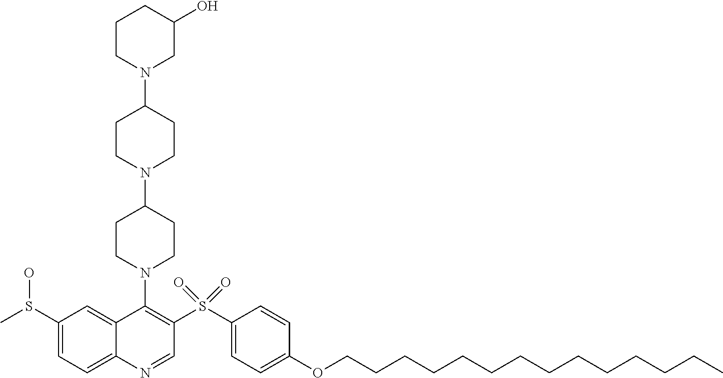 |
| 349 | 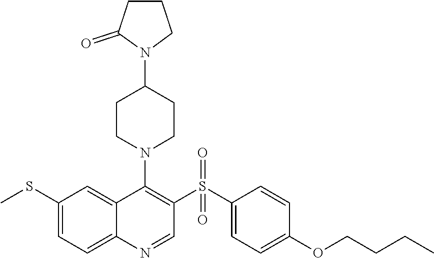 [SMILES: CCCCOc1ccc(S(=O)(=O)c2cnc3ccc(SC)cc3c2N2CCC(N3CCCC3=O)CC2)cc1] |
| 350 | 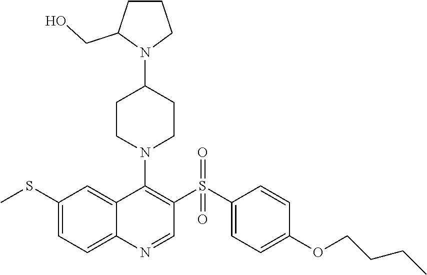 [SMILES: CCCCOc1ccc(S(=O)(=O)c2cnc3ccc(SC)cc3c2N2CCC(N3CCCC3CO)CC2)cc1] |
| 351 | 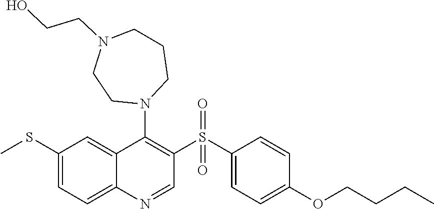 [SMILES: CCCCOc1ccc(S(=O)(=O)c2cnc3ccc(SC)cc3c2N2CCCN(CCO)CC2)cc1] |
| 352 | 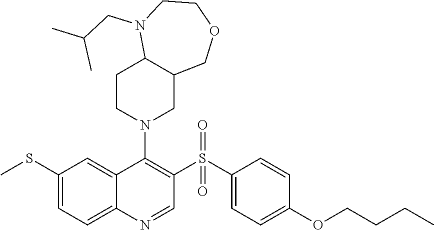 [SMILES: CCCCOc1ccc(S(=O)(=O)c2cnc3ccc(SC)cc3c2N2CCC3C(COCCN3CC(C)C)C2)cc1] |
| 353 | 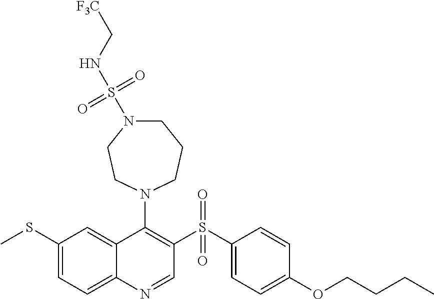 [SMILES: CCCCOc1ccc(S(=O)(=O)c2cnc3ccc(SC)cc3c2N2CCCN(S(=O)(=O)NCC(F)(F)F)CC2)cc1] |
| 354 | 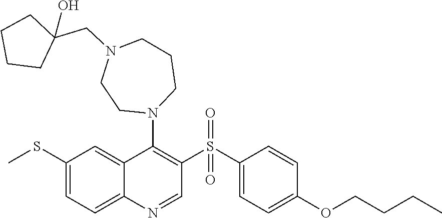 [SMILES: CCCCOc1ccc(S(=O)(=O)c2cnc3ccc(SC)cc3c2N2CCCN(CC3(O)CCCC3)CC2)cc1] |
| 355 | 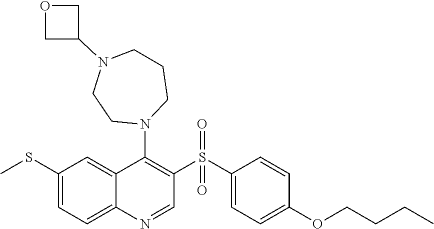 [SMILES: CCCCOc1ccc(S(=O)(=O)c2cnc3ccc(SC)cc3c2N2CCCN(C3COC3)CC2)cc1] |
| 356 | 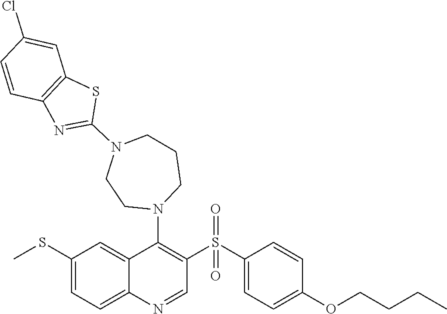 [SMILES: CCCCOc1ccc(S(=O)(=O)c2cnc3ccc(SC)cc3c2N2CCCN(c3nc4ccc(Cl)cc4s3)CC2)cc1] |
| 357 | 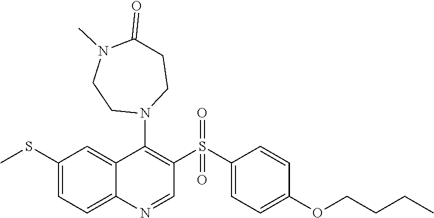 [SMILES: CCCCOc1ccc(S(=O)(=O)c2cnc3ccc(SC)cc3c2N2CCC(=O)N(C)CC2)cc1] |
| 358 | 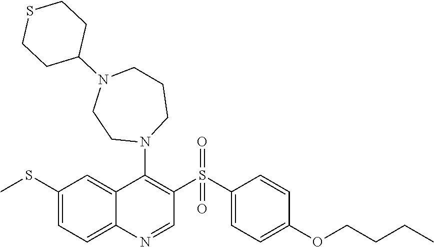 [SMILES: CCCCOc1ccc(S(=O)(=O)c2cnc3ccc(SC)cc3c2N2CCCN(C3CCSCC3)CC2)cc1] |
| 359 | 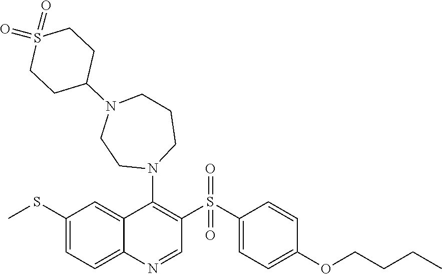 [SMILES: CCCCOc1ccc(S(=O)(=O)c2cnc3ccc(SC)cc3c2N2CCCN(C3CCS(=O)(=O)CC3)CC2)cc1] |
| 360 | 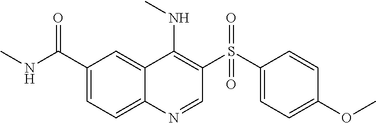 |
| 361 | 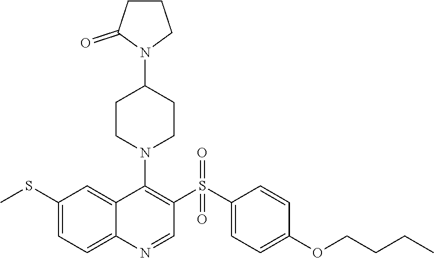 [SMILES: CCCCOc1ccc(S(=O)(=O)c2cnc3ccc(SC)cc3c2N2CCC(N3CCCC3=O)CC2)cc1] |
| 362 | 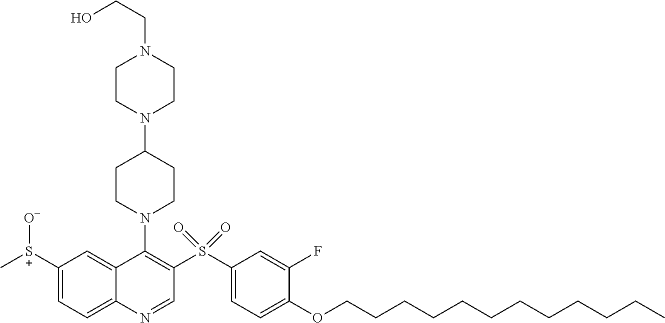 [SMILES: CCCCCCCCCCCCOc1ccc(S(=O)(=O)c2cnc3ccc([S+](C)[O-])cc3c2N2CCC(N3CCN(CCO)CC3)CC2)cc1F] |
| 363 | 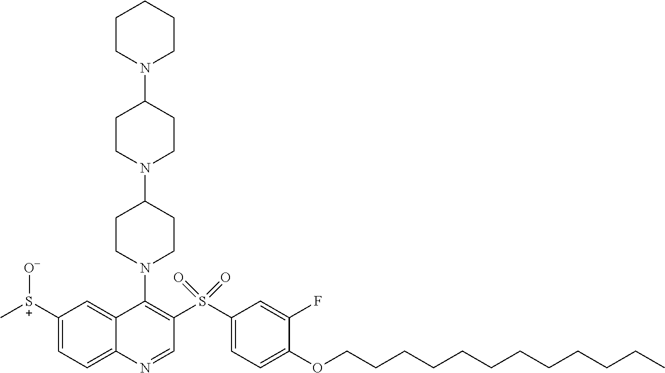 [SMILES: CCCCCCCCCCCCOc1ccc(S(=O)(=O)c2cnc3ccc([S+](C)[O-])cc3c2N2CCC(N3CCC(N4CCCCC4)CC3)CC2)cc1F] |
| 364 | 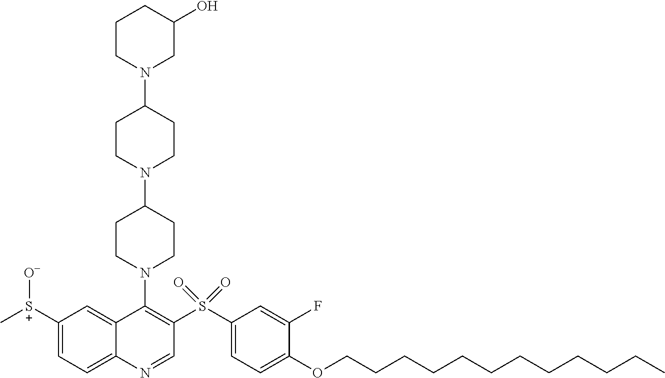 [SMILES: CCCCCCCCCCCCOc1ccc(S(=O)(=O)c2cnc3ccc([S+](C)[O-])cc3c2N2CCC(N3CCC(N4CCCC(O)C4)CC3)CC2)cc1F] |
| 365 | 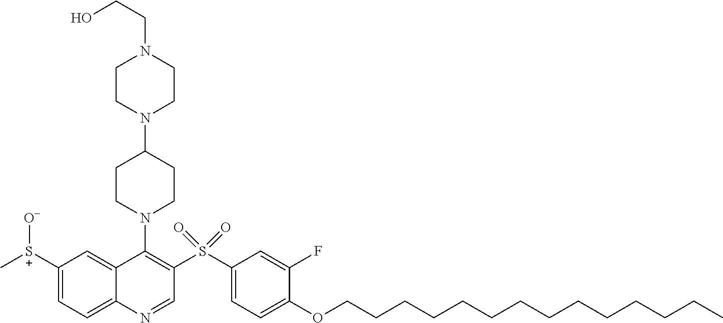 [SMILES: CCCCCCCCCCCCCCOc1ccc(S(=O)(=O)c2cnc3ccc([S+](C)[O-])cc3c2N2CCC(N3CCN(CCO)CC3)CC2)cc1F] |
| 366 | 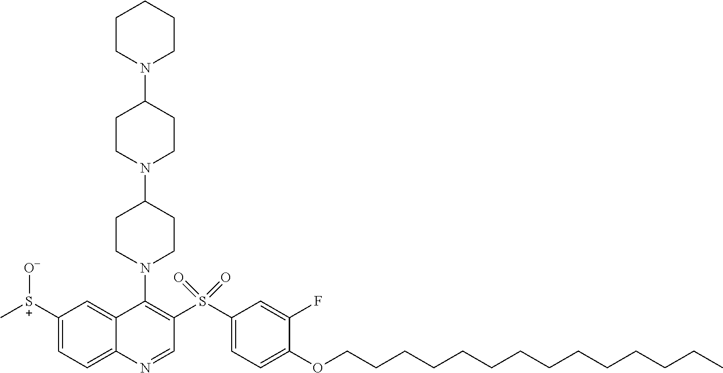 [SMILES: CCCCCCCCCCCCCCOc1ccc(S(=O)(=O)c2cnc3ccc([S+](C)[O-])cc3c2N2CCC(N3CCC(N4CCCCC4)CC3)CC2)cc1F] |
| 367 | 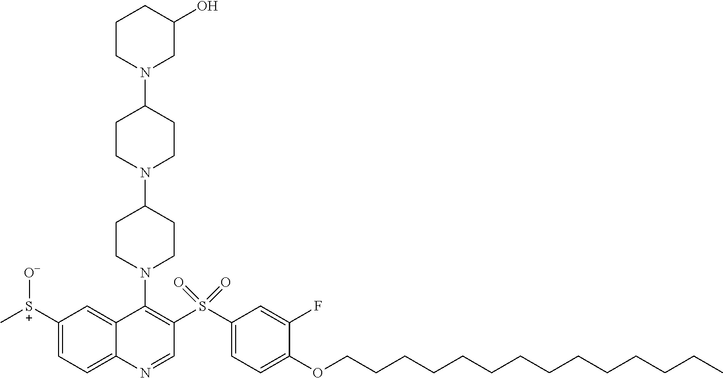 [SMILES: CCCCCCCCCCCCCCOc1ccc(S(=O)(=O)c2cnc3ccc([S+](C)[O-])cc3c2N2CCC(N3CCC(N4CCCC(O)C4)CC3)CC2)cc1F] |
| 368 | 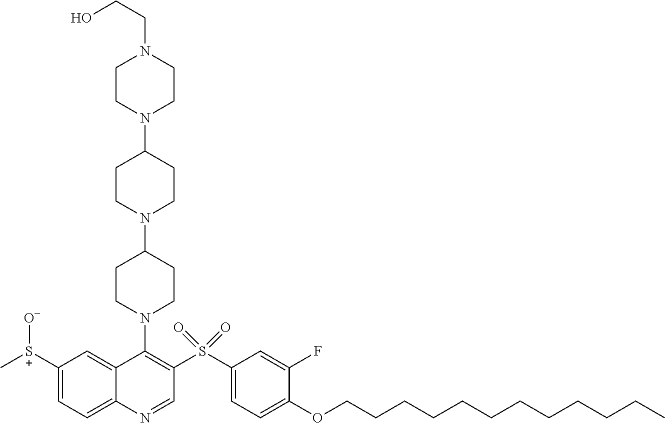 [SMILES: CCCCCCCCCCCCOc1ccc(S(=O)(=O)c2cnc3ccc([S+](C)[O-])cc3c2N2CCC(N3CCC(N4CCN(CCO)CC4)CC3)CC2)cc1F] |
| 369 | 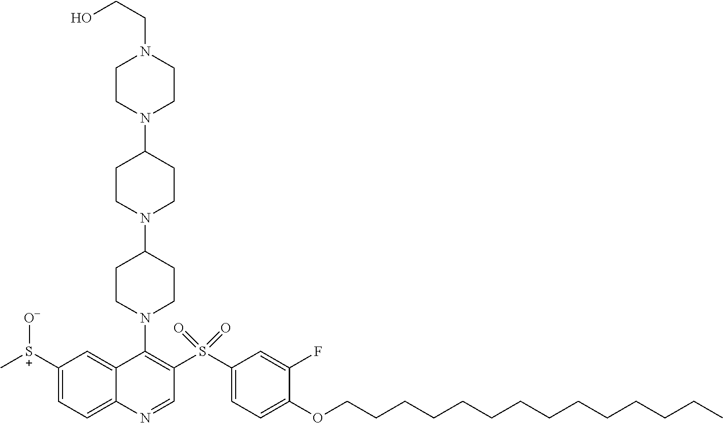 [SMILES: CCCCCCCCCCCCCCOc1ccc(S(=O)(=O)c2cnc3ccc([S+](C)[O-])cc3c2N2CCC(N3CCC(N4CCN(CCO)CC4)CC3)CC2)cc1F] |
| 370 | 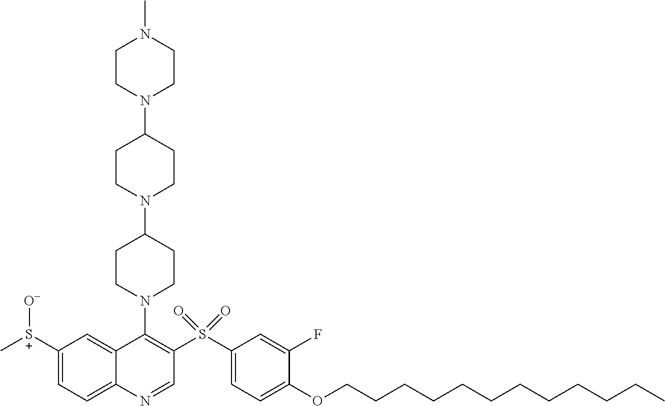 [SMILES: CCCCCCCCCCCCOc1ccc(S(=O)(=O)c2cnc3ccc([S+](C)[O-])cc3c2N2CCC(N3CCC(N4CCN(C)CC4)CC3)CC2)cc1F] |
| 371 | 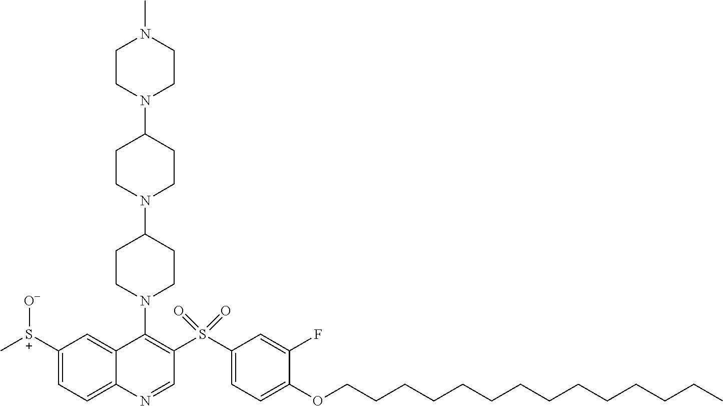 [SMILES: CCCCCCCCCCCCCCOc1ccc(S(=O)(=O)c2cnc3ccc([S+](C)[O-])cc3c2N2CCC(N3CCC(N4CCN(C)CC4)CC3)CC2)cc1F] |
| 372 | 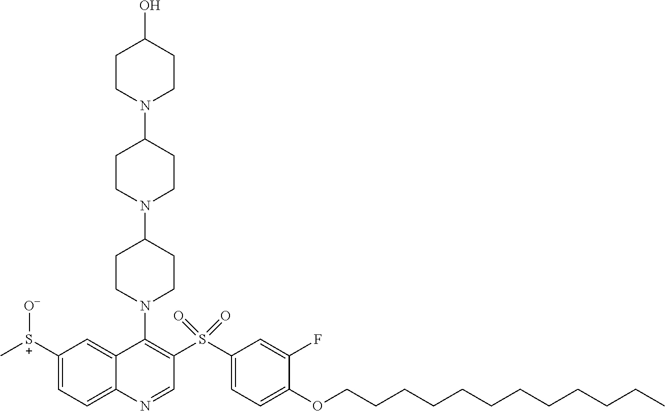 [SMILES: CCCCCCCCCCCCOc1ccc(S(=O)(=O)c2cnc3ccc([S+](C)[O-])cc3c2N2CCC(N3CCC(N4CCC(O)CC4)CC3)CC2)cc1F] |
| 373 | 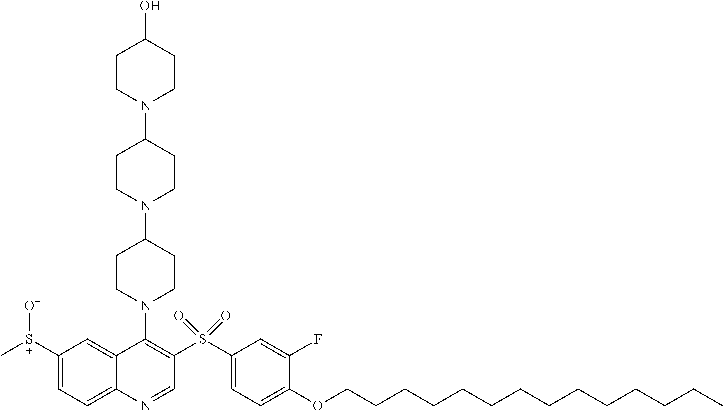 [SMILES: CCCCCCCCCCCCCCOc1ccc(S(=O)(=O)c2cnc3ccc([S+](C)[O-])cc3c2N2CCC(N3CCC(N4CCC(O)CC4)CC3)CC2)cc1F] |
| 374 | 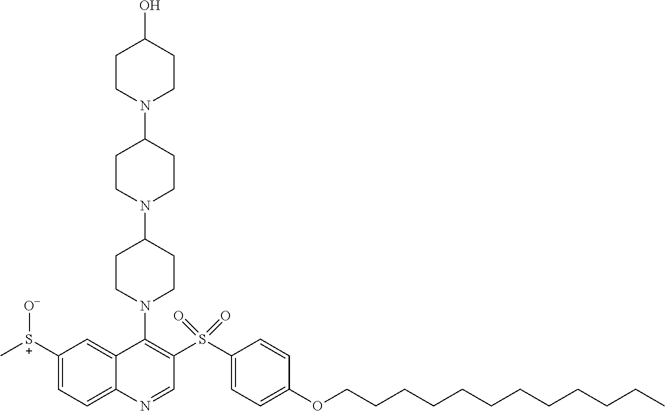 [SMILES: CCCCCCCCCCCCOc1ccc(S(=O)(=O)c2cnc3ccc([S+](C)[O-])cc3c2N2CCC(N3CCC(N4CCC(O)CC4)CC3)CC2)cc1] |
| 375 | 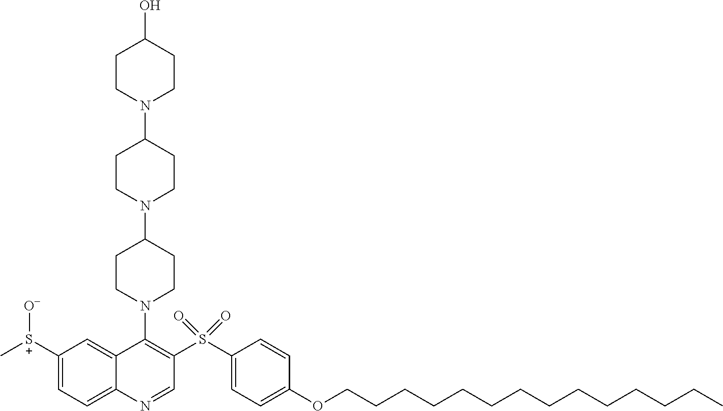 [SMILES: CCCCCCCCCCCCCCOc1ccc(S(=O)(=O)c2cnc3ccc([S+](C)[O-])cc3c2N2CCC(N3CCC(N4CCC(O)CC4)CC3)CC2)cc1] |
| 376 | 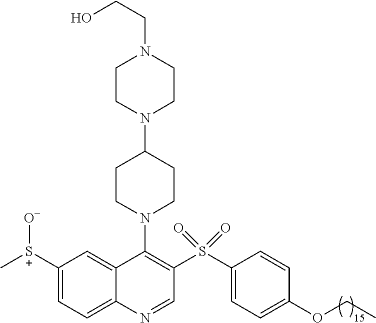 |
| 377 | 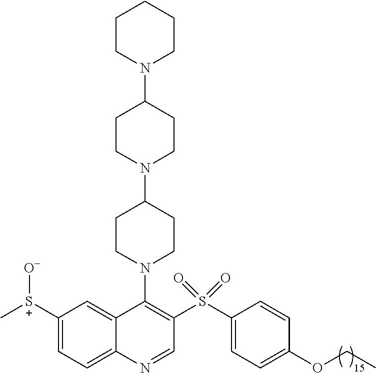 |
| 378 | 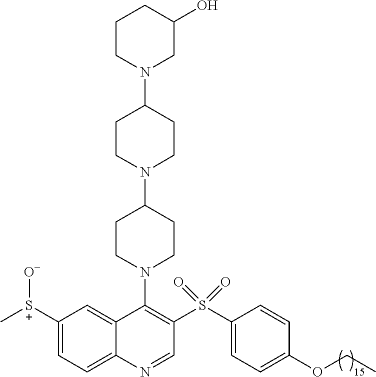 |
| 379 | 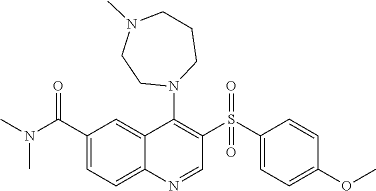 |
| 380 | 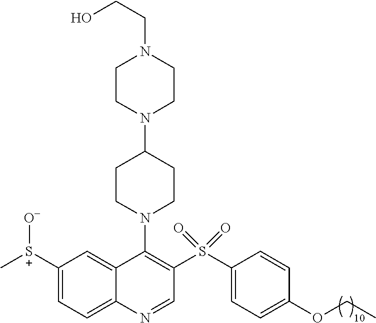 |
| 381 | 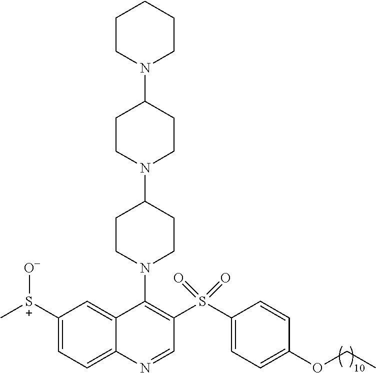 |
| 382 | 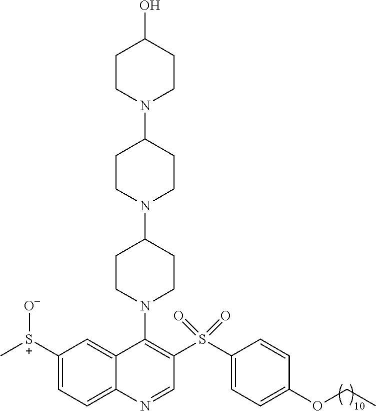 |
| 383 | 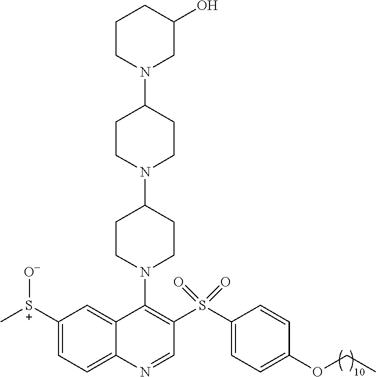 |
| 384 | 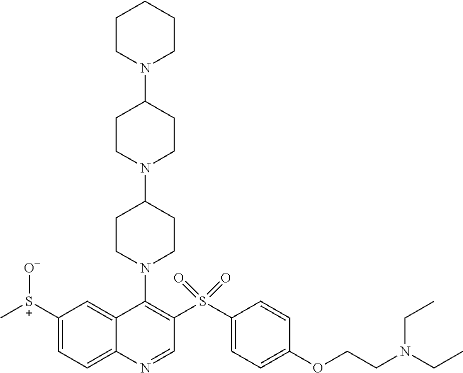 [SMILES: CCN(CC)CCOc1ccc(S(=O)(=O)c2cnc3ccc([S+](C)[O-])cc3c2N2CCC(N3CCC(N4CCCCC4)CC3)CC2)cc1] |
| 385 | 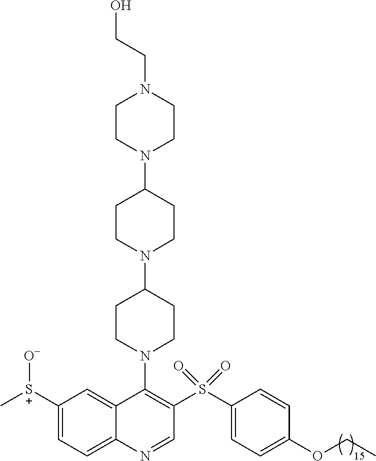 |
| 386 | 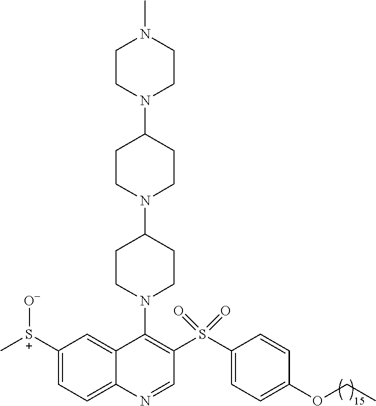 |
| 387 | 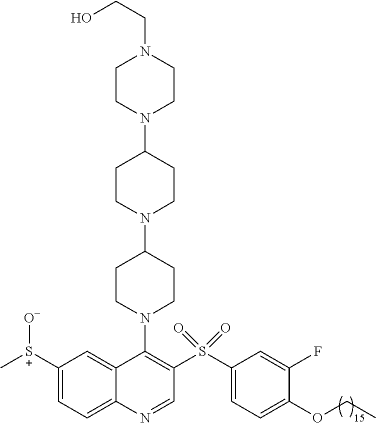 |
| 388 | 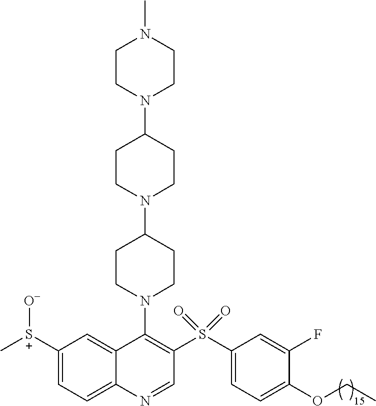 |
| 389 | 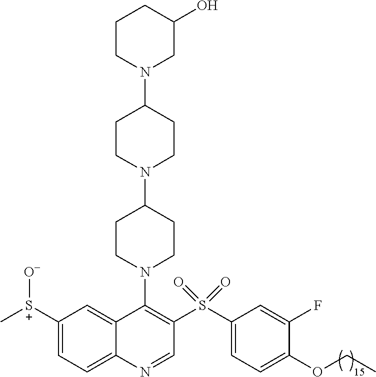 |
| 390 | 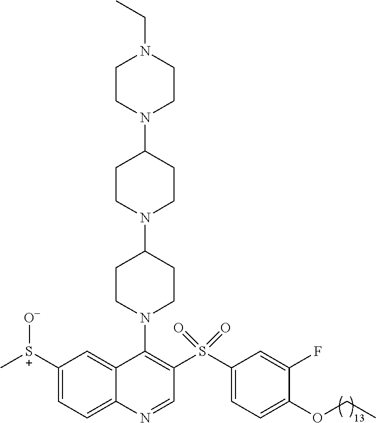 |
| 391 | 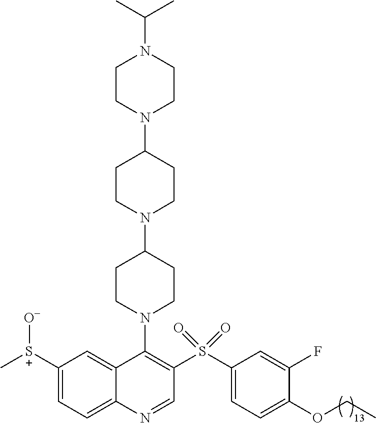 |
| 392 | 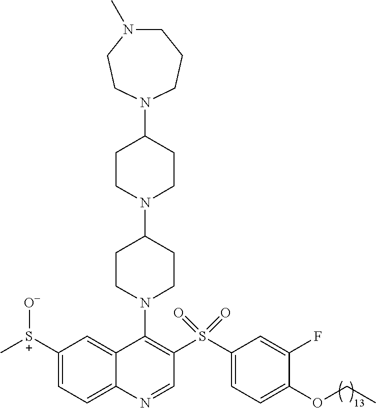 |
| 393 | 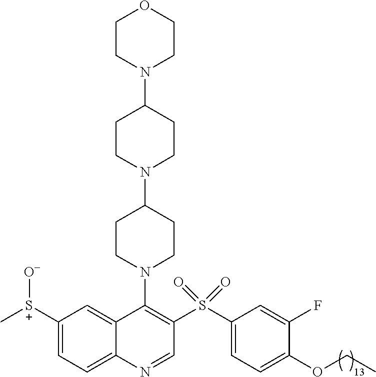 |
| 394 | 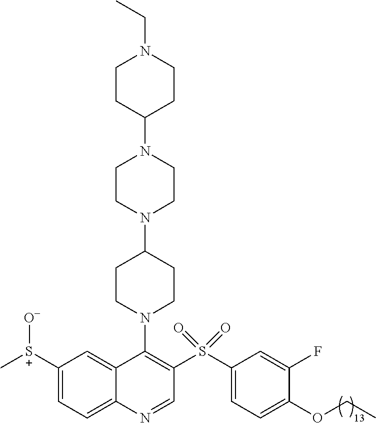 |
| 395 | 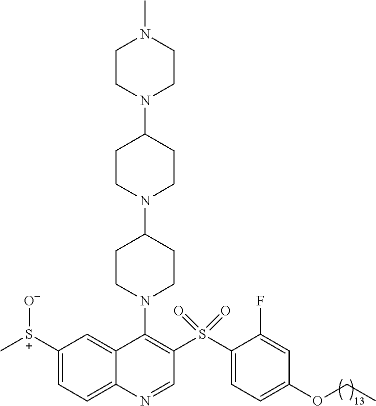 |
| 396 | 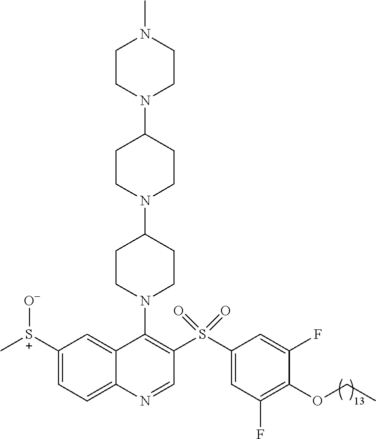 |
| 397 | 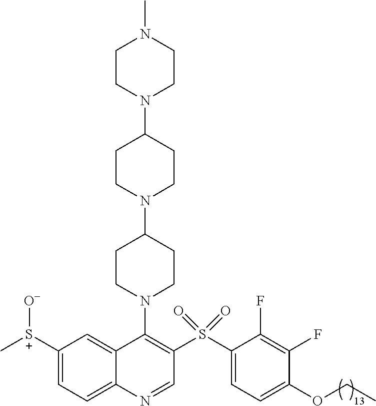 |
| 398 | 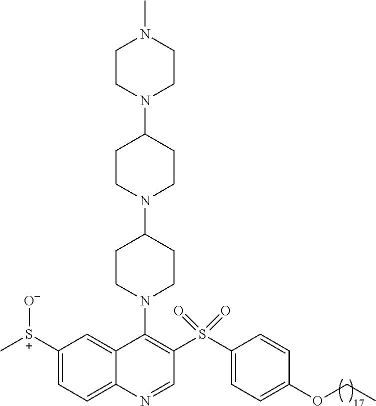 |
| 399 | 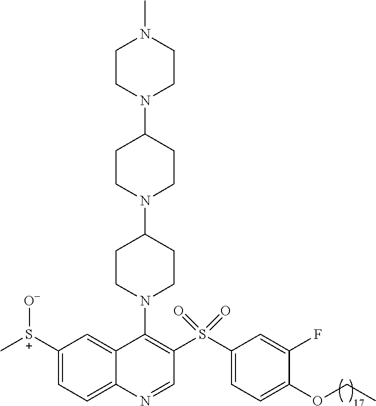 |
| 400 | 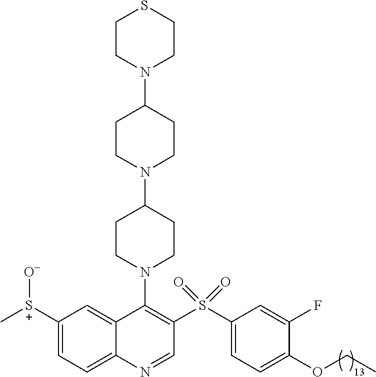 |
| 401 | 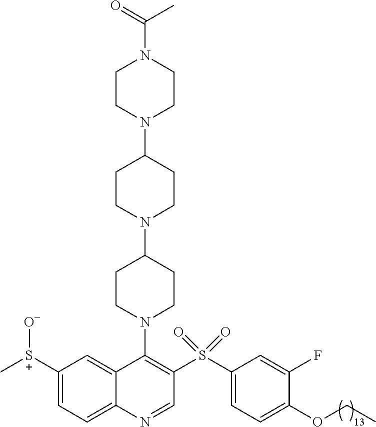 |
| 402 | 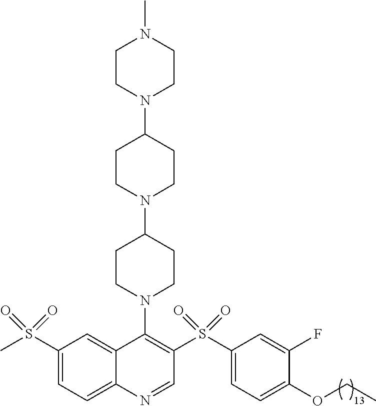 |
| 403 | 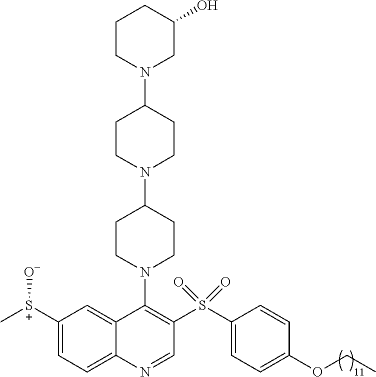 |
| 404 | 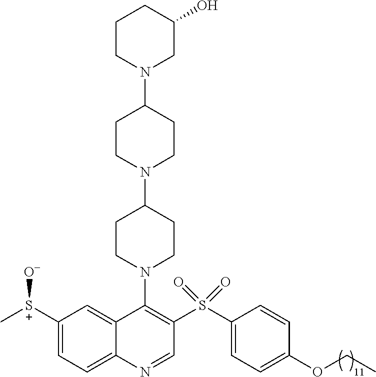 |
| 405 | 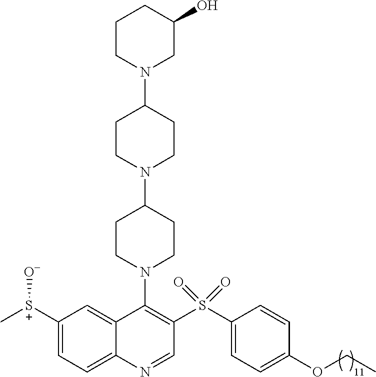 |
| 406 | 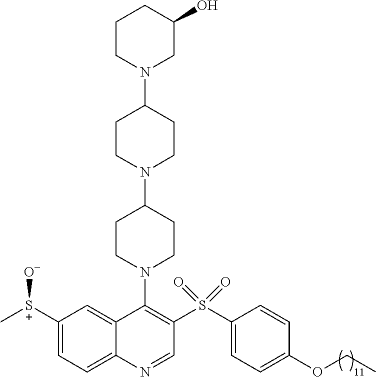 |
| 407 | 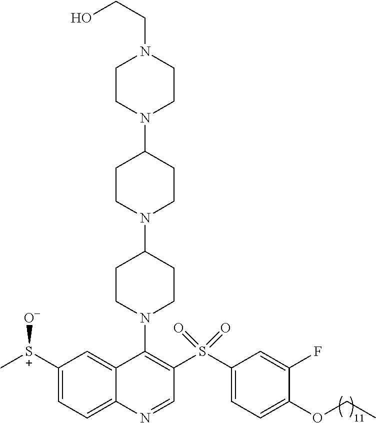 |
| 408 | 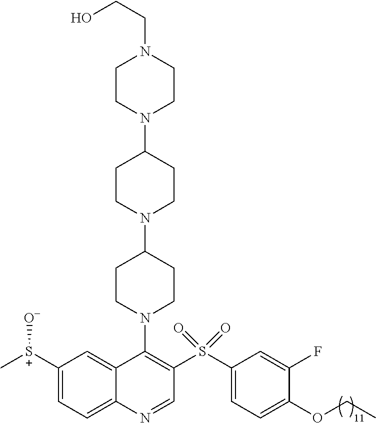 |
| 409 | 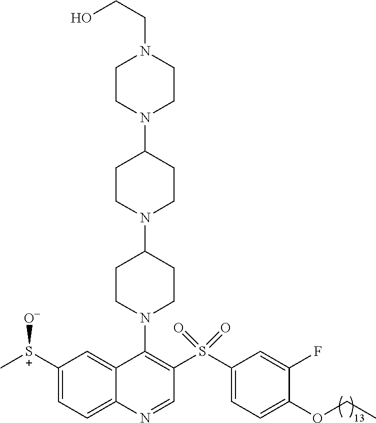 |
| 410 | 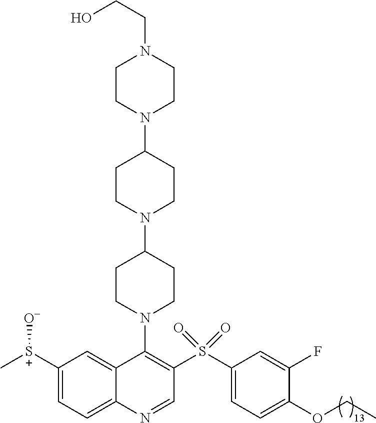 |
| 411 | 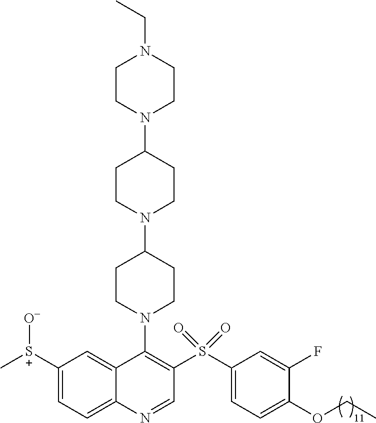 |
| 412 | 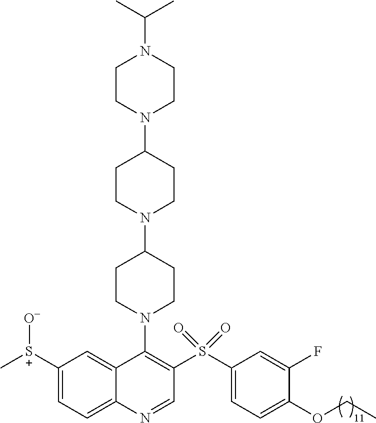 |
| 413 | 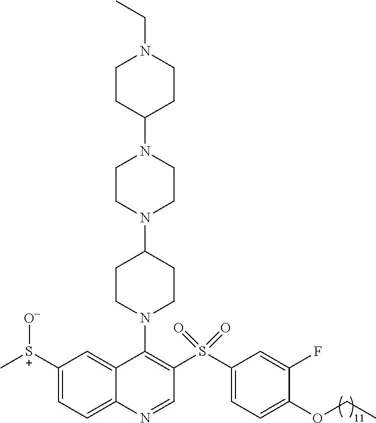 |
| 414 | 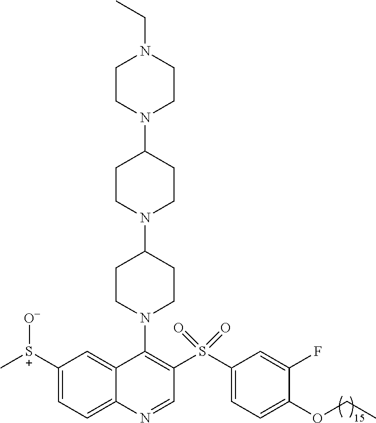 |
| 415 | 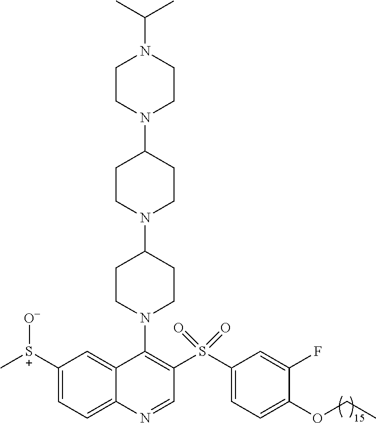 |
| 416 | 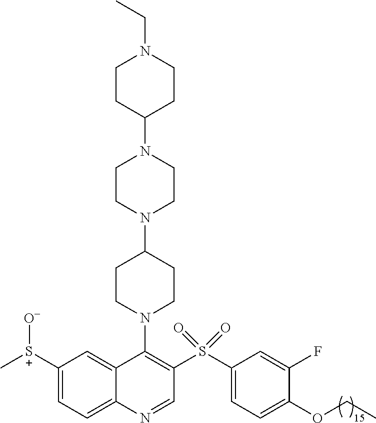 |
| 417 | 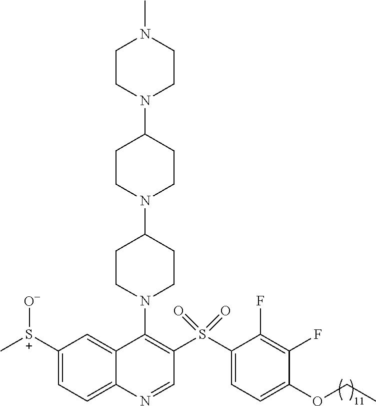 |
| 418 | 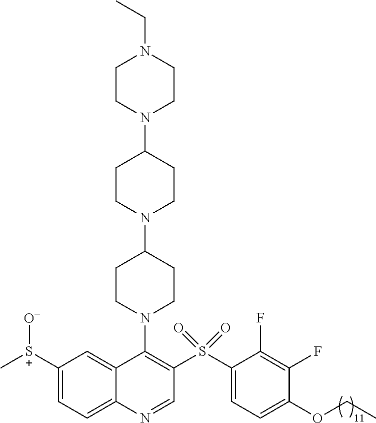 |
| 419 | 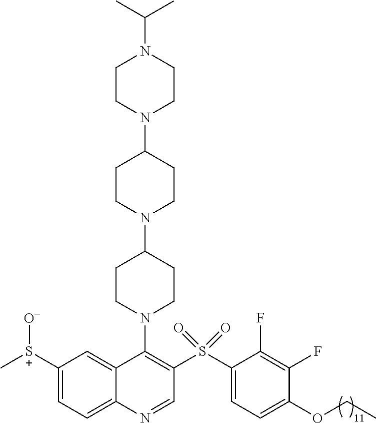 |
| 420 | 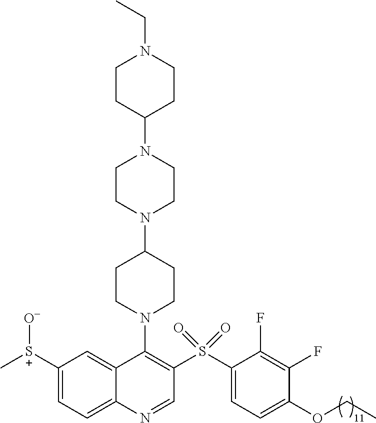 |
| 421 | 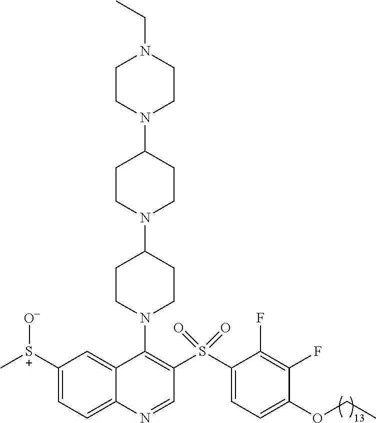 |
| 422 | 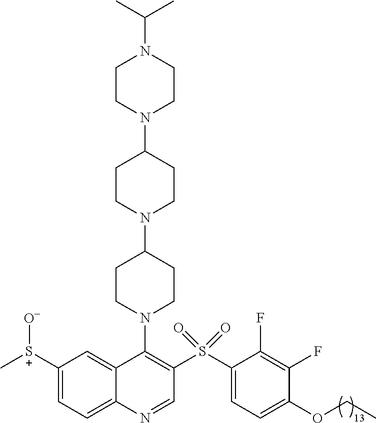 |
| 423 | 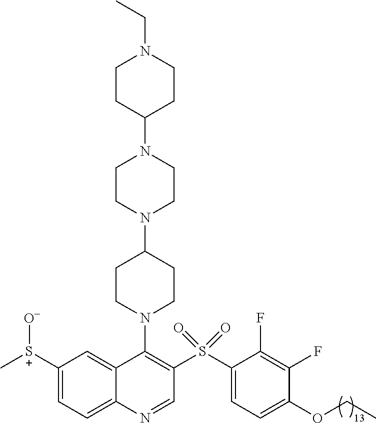 |
| 424 | 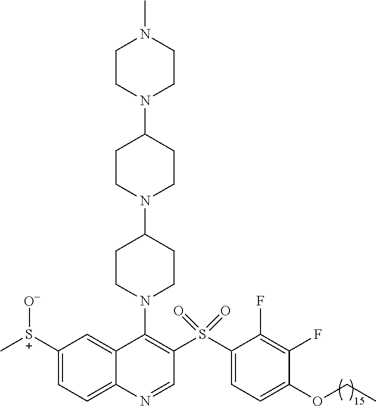 |
| 425 | 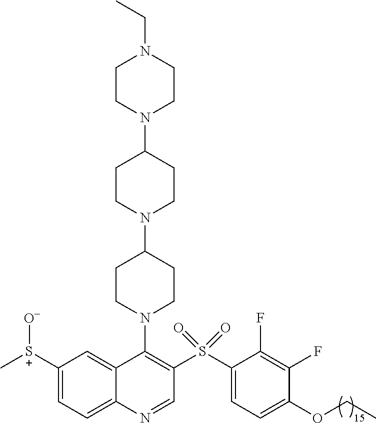 |
| 426 | 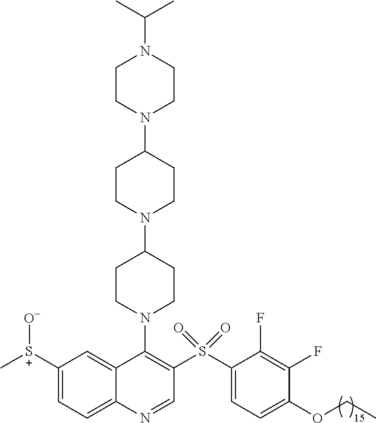 |
| 427 | 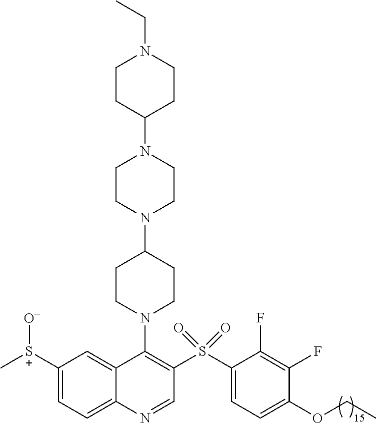 |
| 428 | 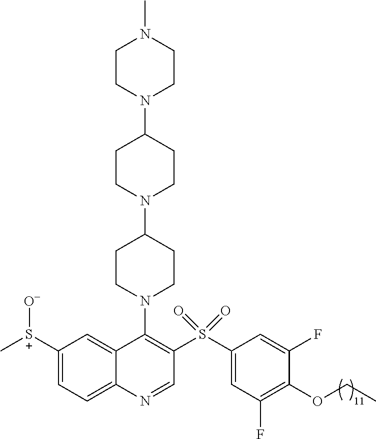 |
| 429 | 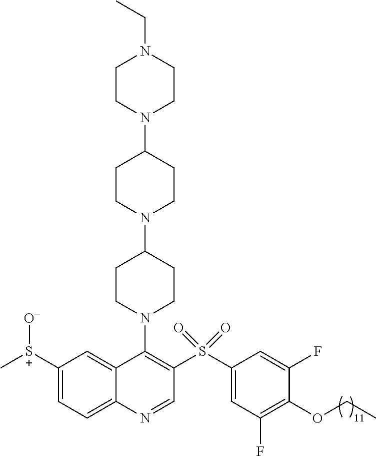 |
| 430 | 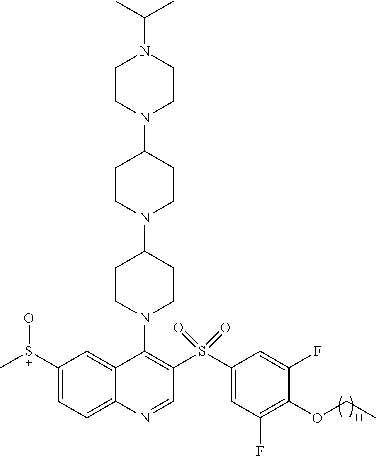 |
| 431 | 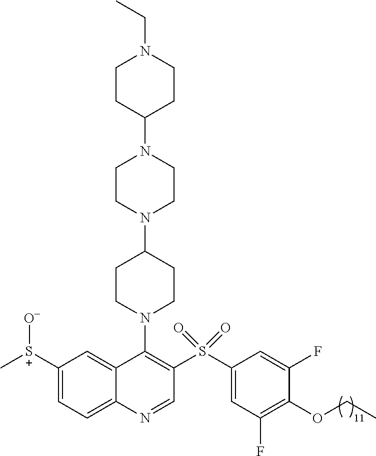 |
| 432 | 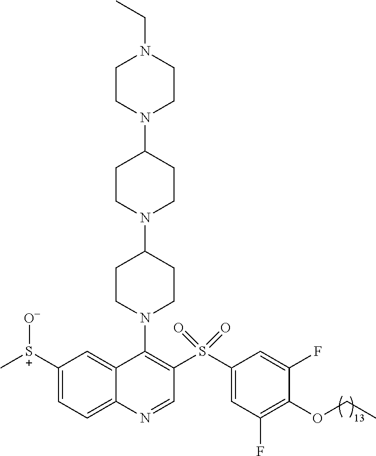 |
| 433 | 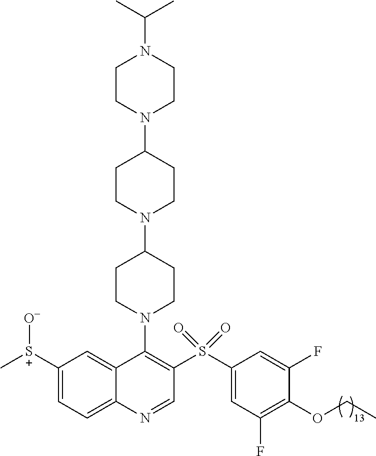 |
| 434 | 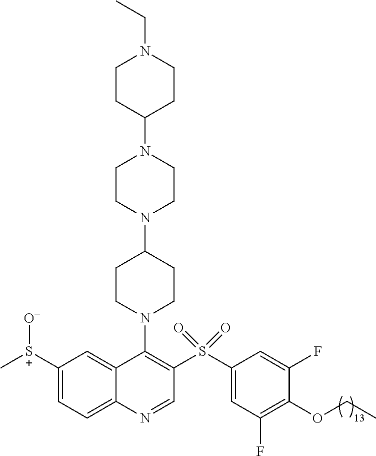 |
| 435 | 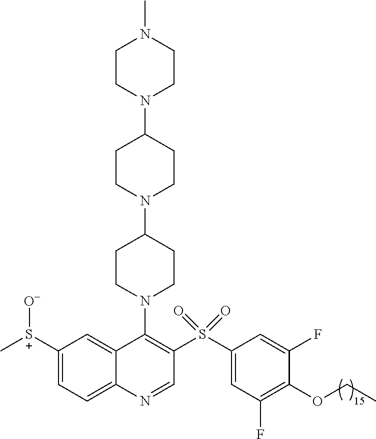 |
| 436 | 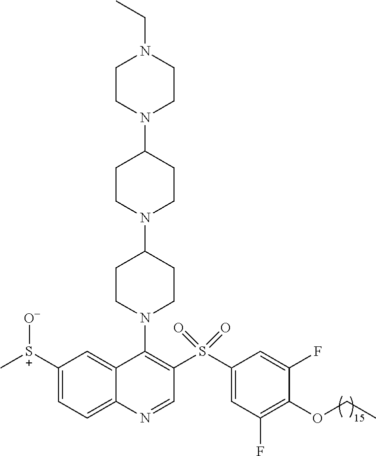 |
| 437 | 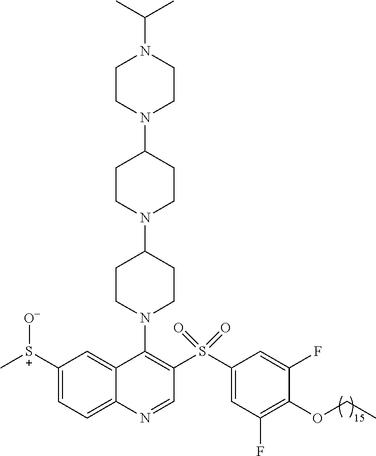 |
| 438 | 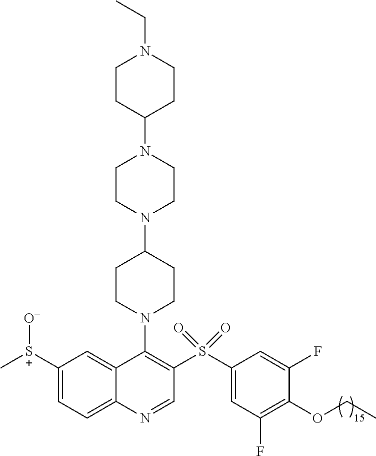 |
| 439 | 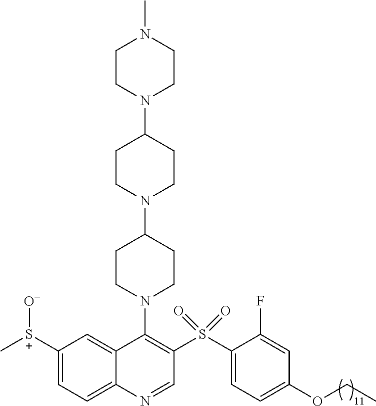 |
| 440 | 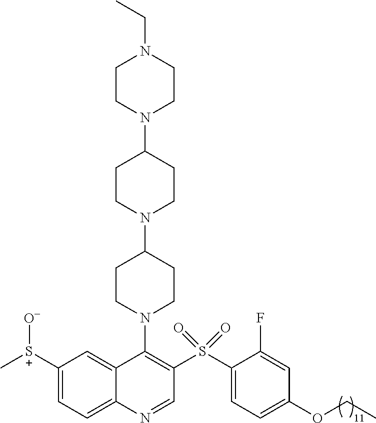 |
| 441 | 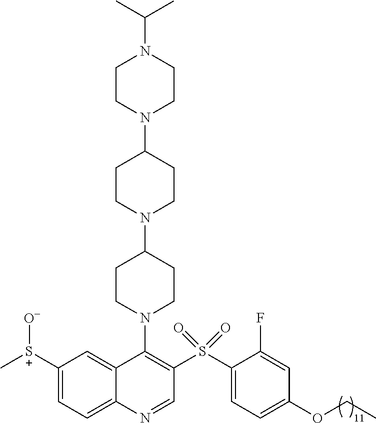 |
| 442 | 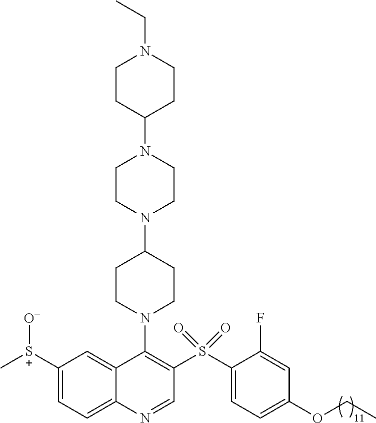 |
| 443 | 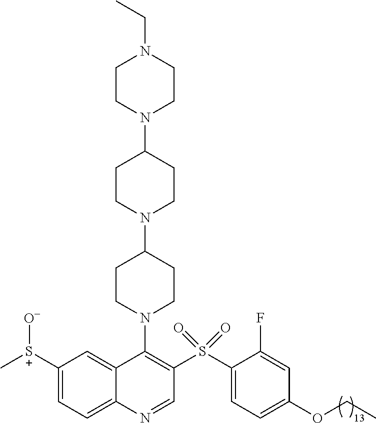 |
| 444 | 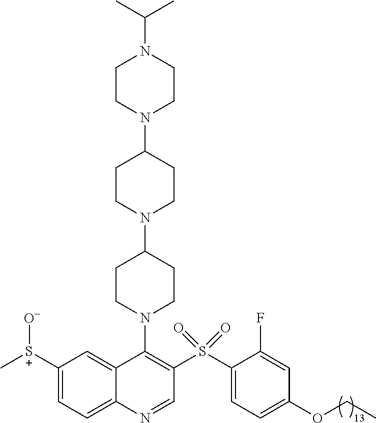 |
| 445 | 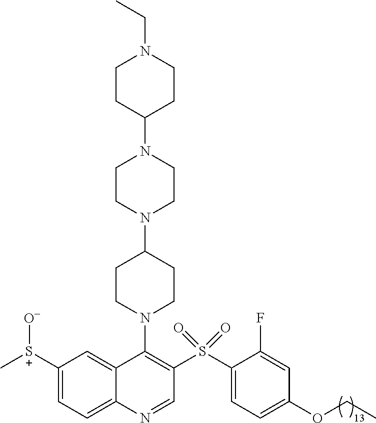 |
| 446 | 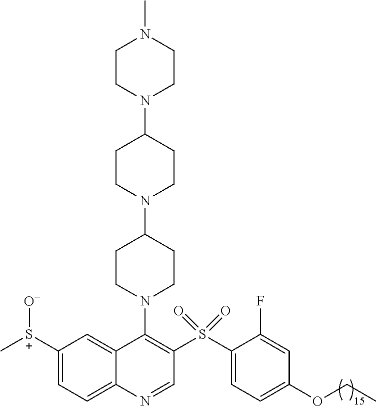 |
| 447 | 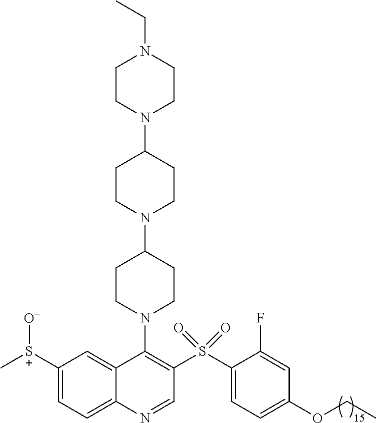 |
| 448 | 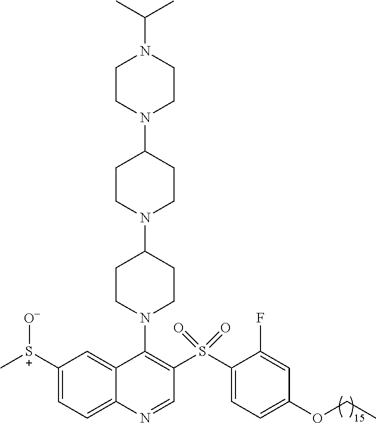 |
| 449 | 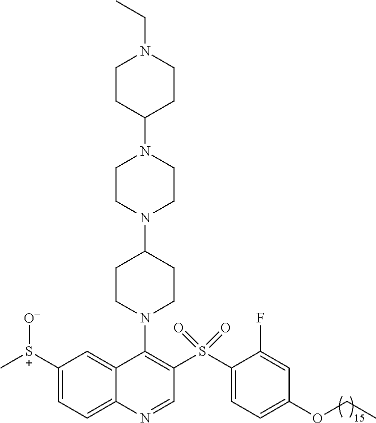 |
| 450 | 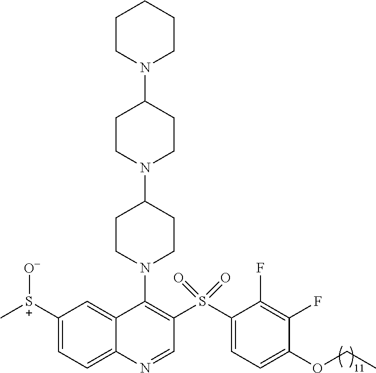 |
| 451 | 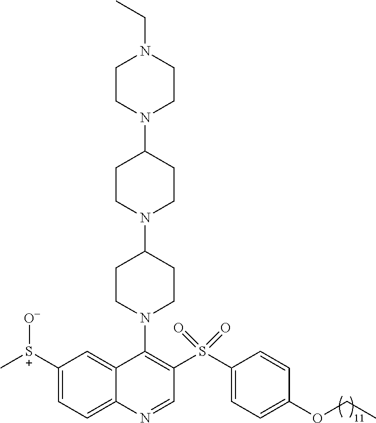 |
| 452 | 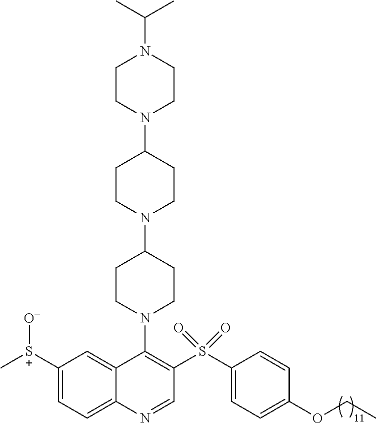 |
| 453 | 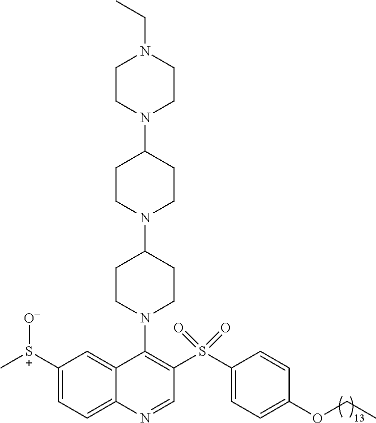 |
| 454 | 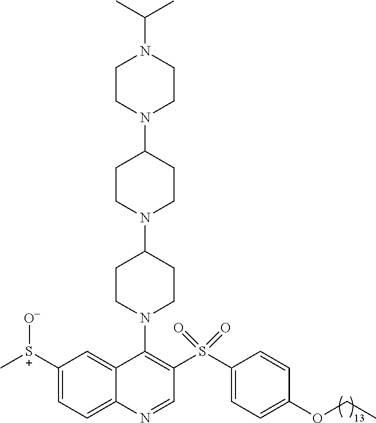 |
| 455 | 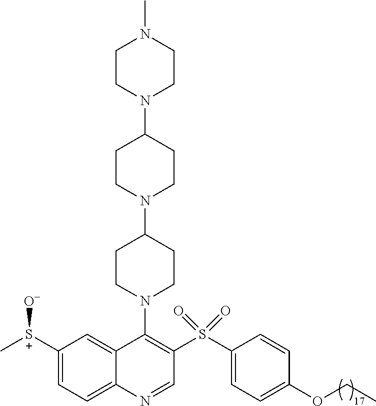 |
| 456 | 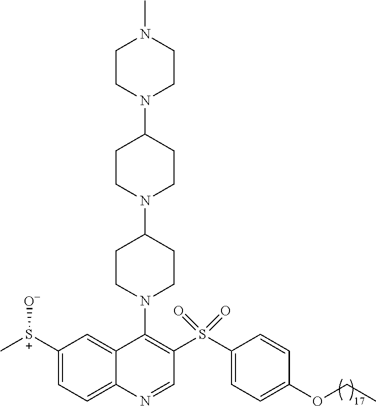 |
| 457 | 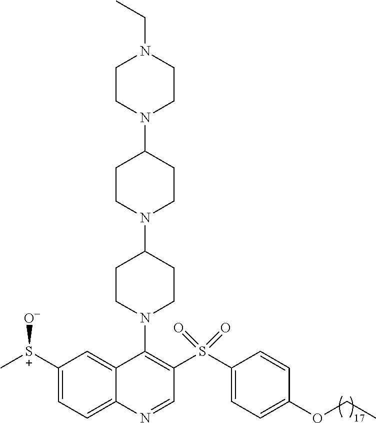 |
| 458 | 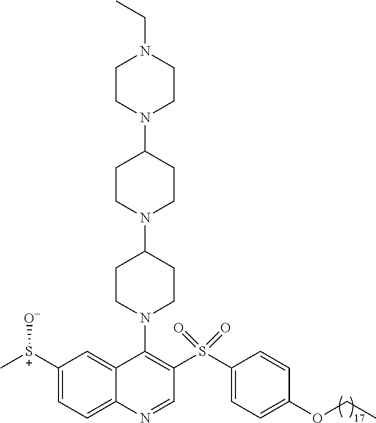 |
| 459 | 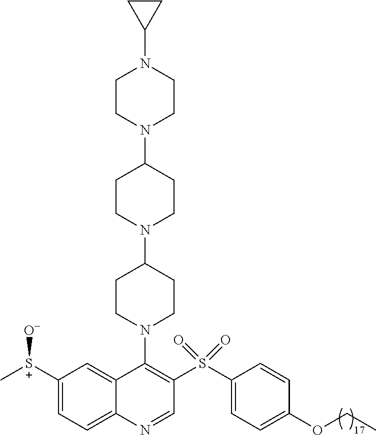 |
| 460 | 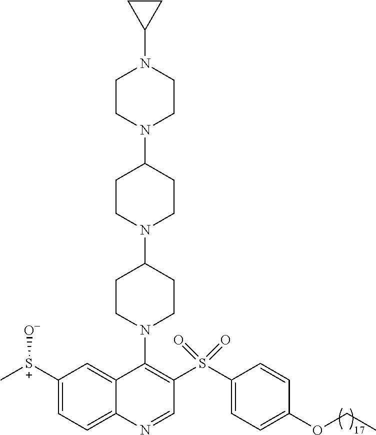 |
| 461 | 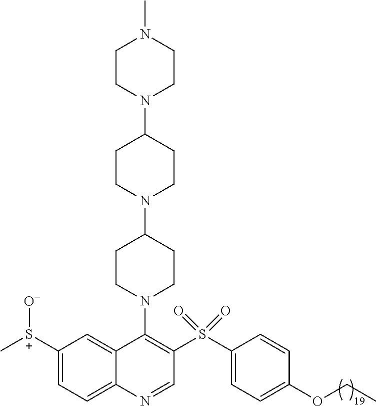 |
| 462 | 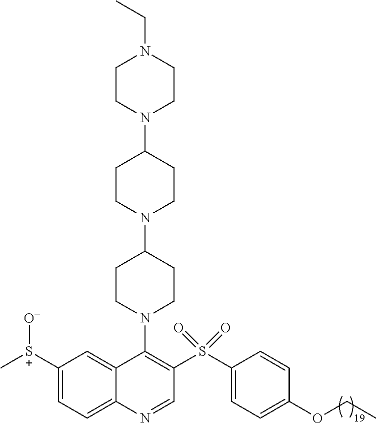 |
| 463 | 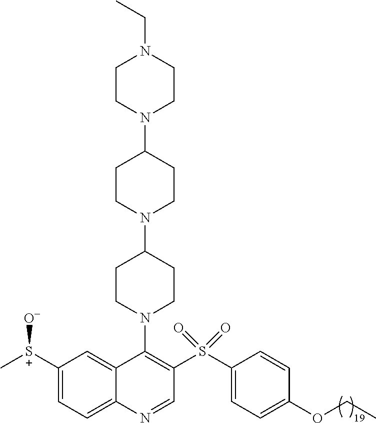 |
| 464 | 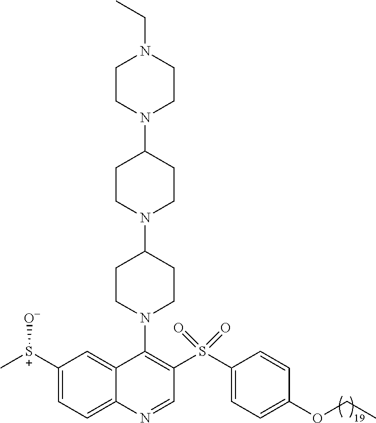 |
| 465 | 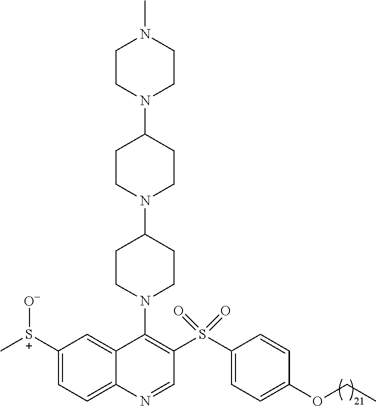 |
| 466 | 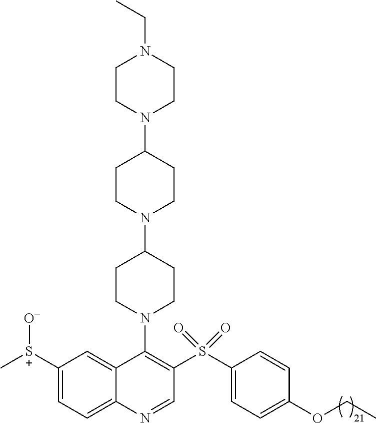 |
| 467 | 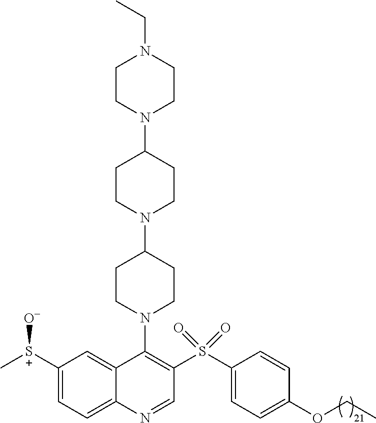 |
| 468 | 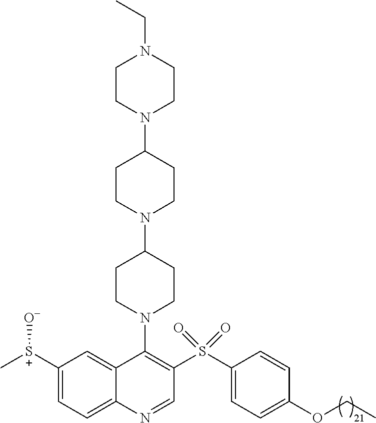 |
| 469 | 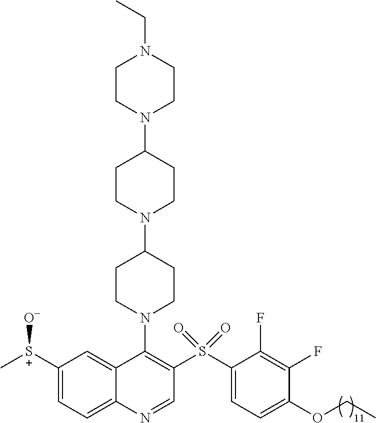 |
| 470 | 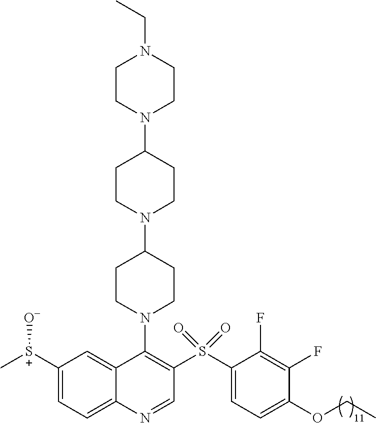 |
| 471 | 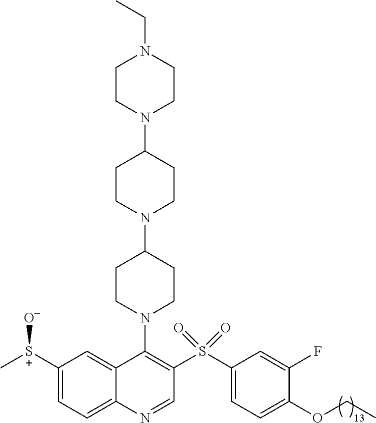 |
| 472 | 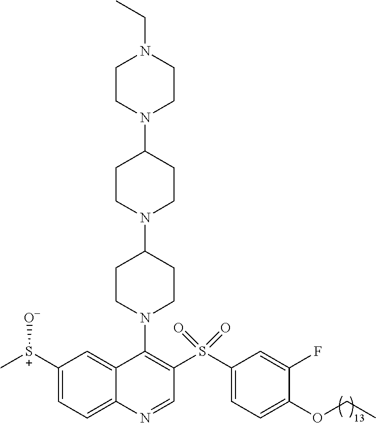 |
| 473 | 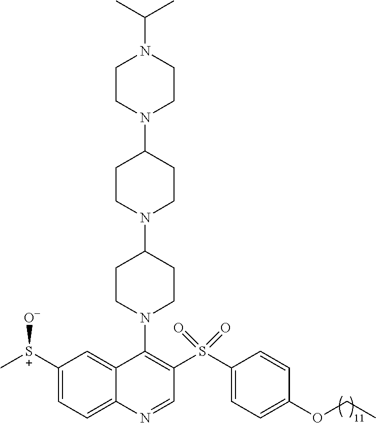 |
| 474 | 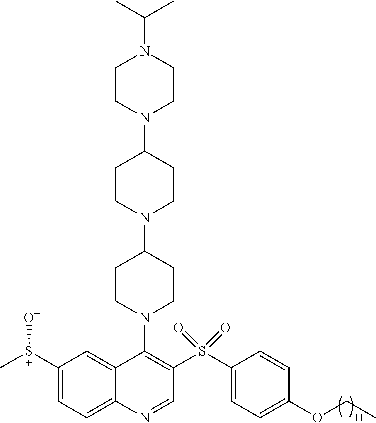 |
| 475 | 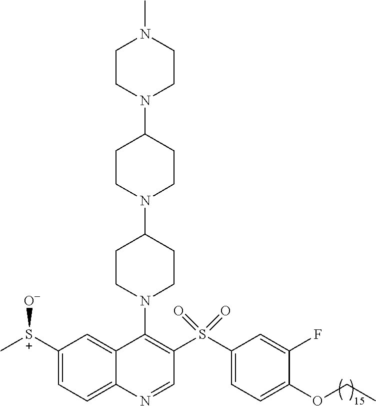 |
| 476 | 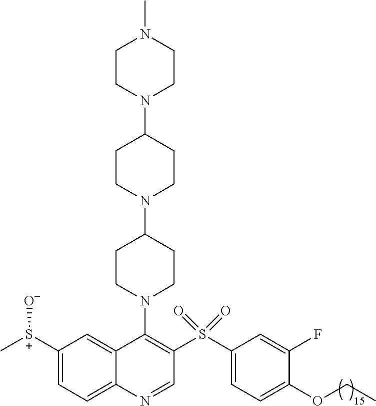 |
| 477 | 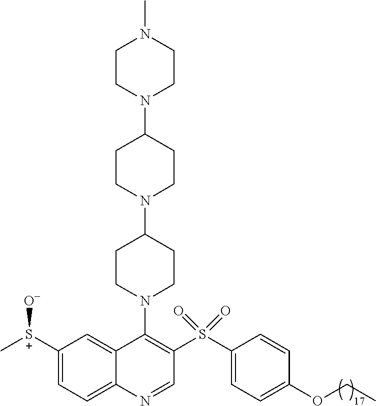 |
| 478 | 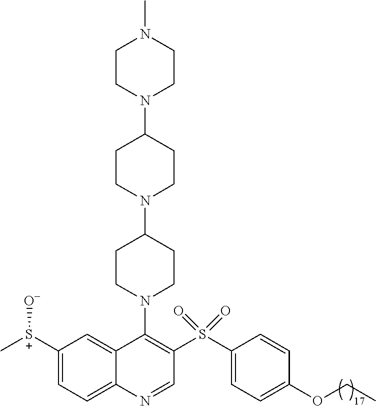 |
| 479 | 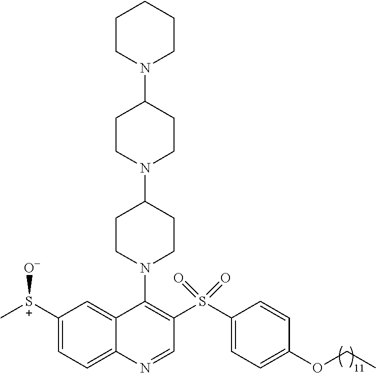 |
| 480 | 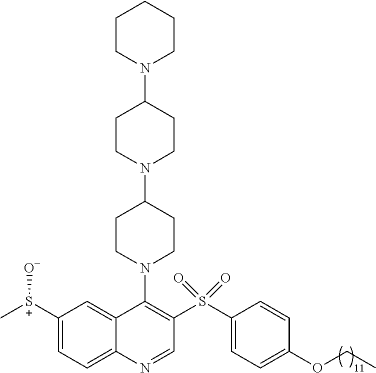 |
| 481 | 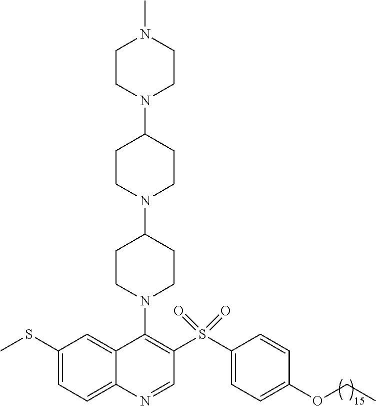 |
| 482 | 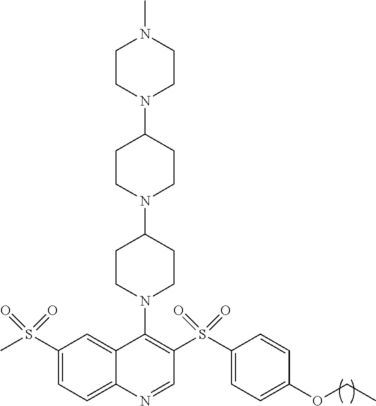 |
| 483 | 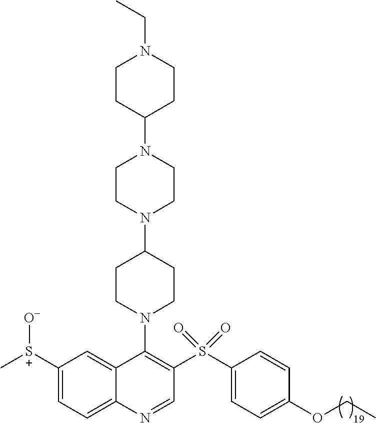 |
| 484 | 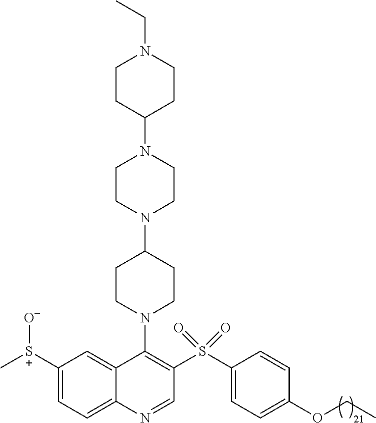 |
| 485 | 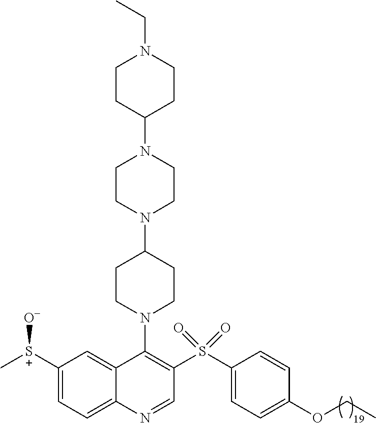 |
| 486 | 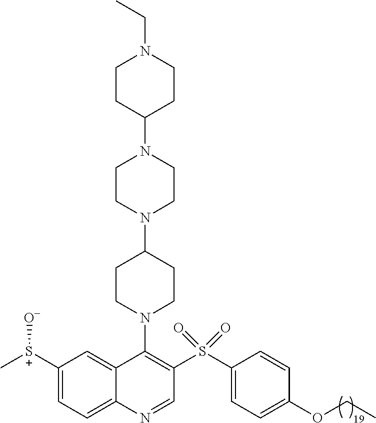 |
| 487 | 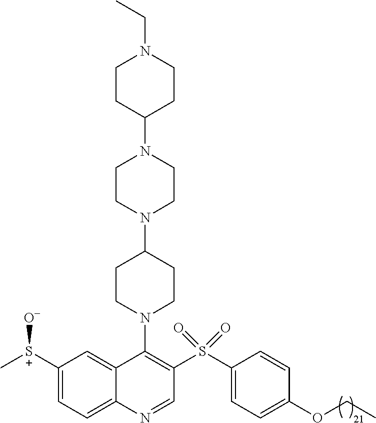 |
| 488 | 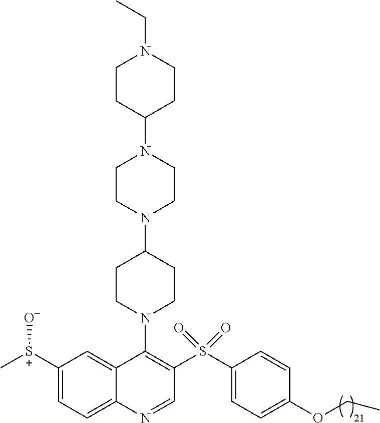 |
| 489 | 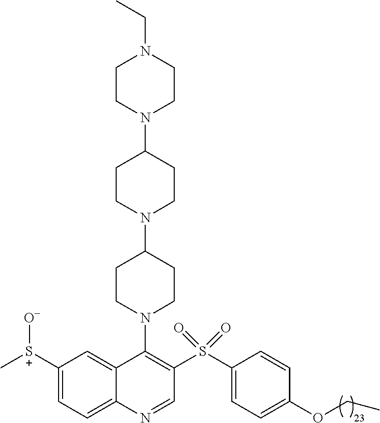 |
| 490 | 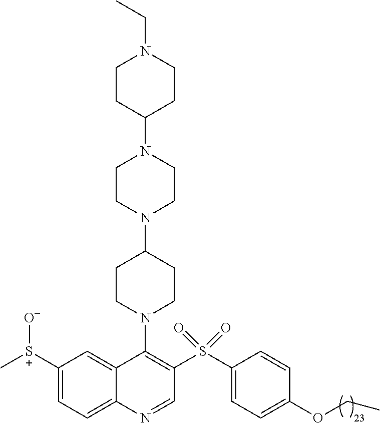 |
| 491 | 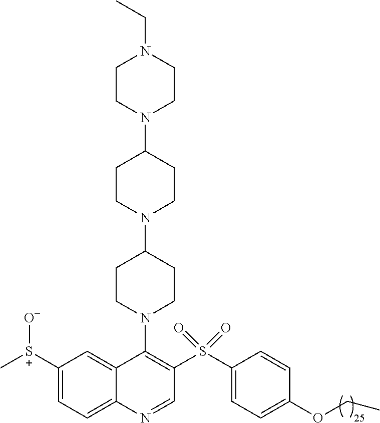 |
| 492 | 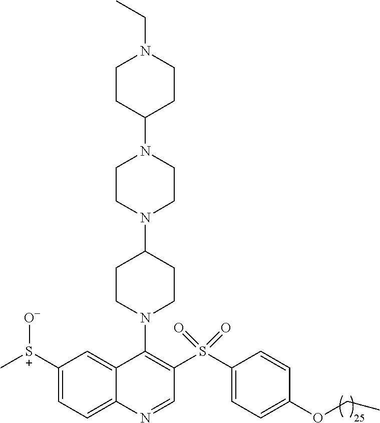 |
| 493 | 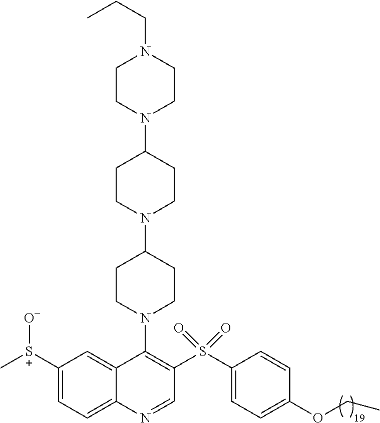 |
| 494 | 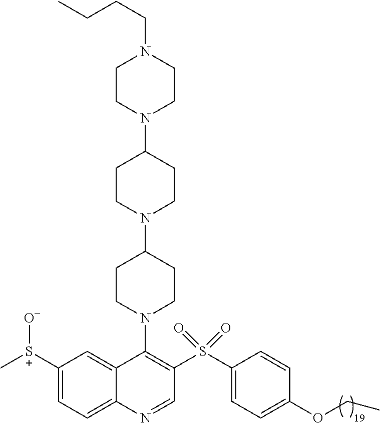 |
| 495 | 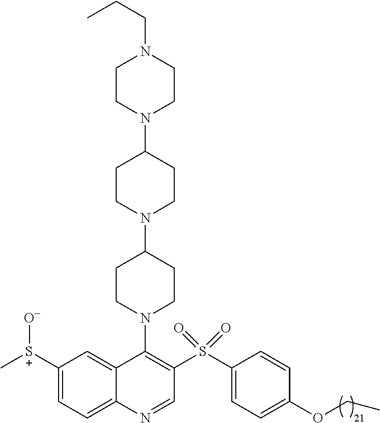 |
| 496 | 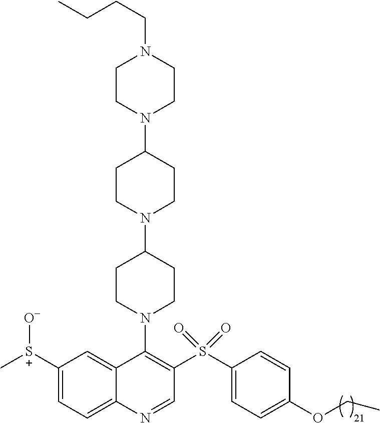 |
| 497 | 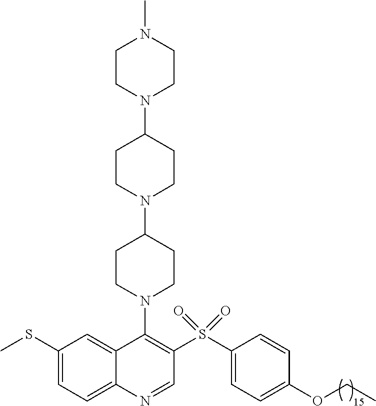 |
| 498 | 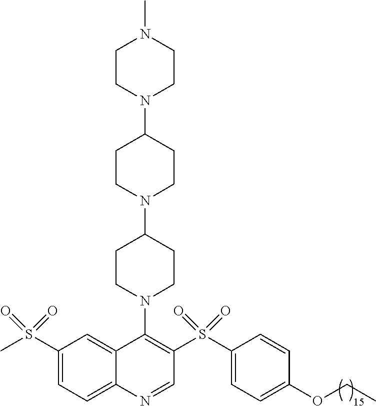 |
| 499 | 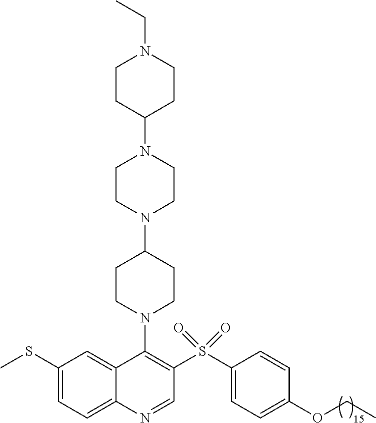 |
| 500 | 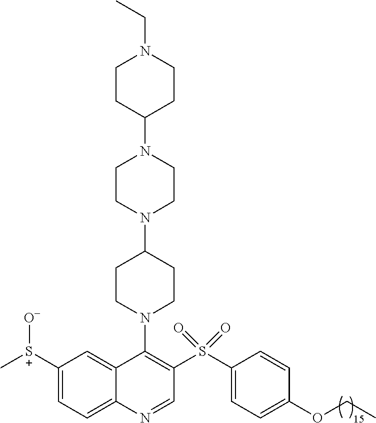 |
| 501 | 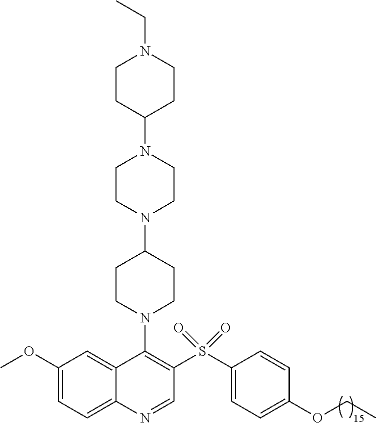 |
| 502 | 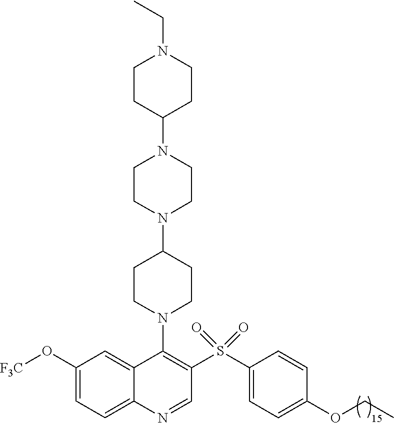 |
| 503 | 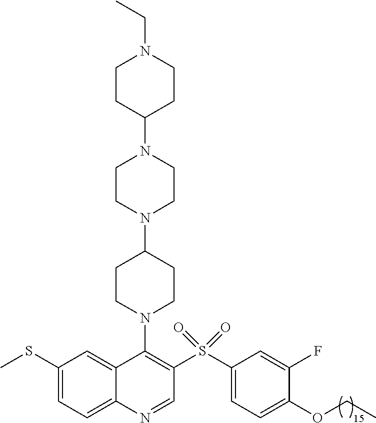 |
| 504 | 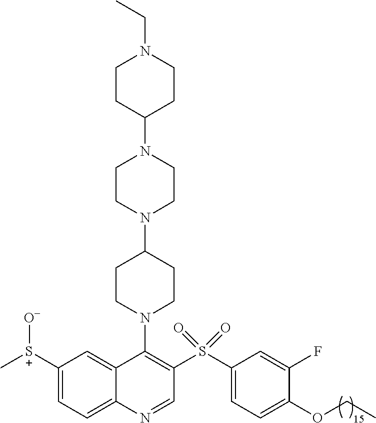 |
| 505 | 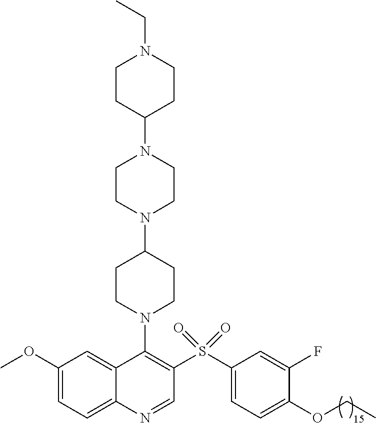 |
| 506 | 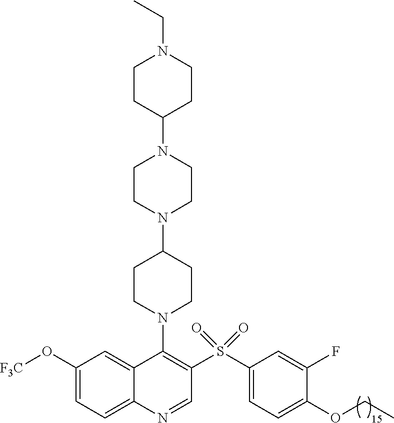 |
| 507 | 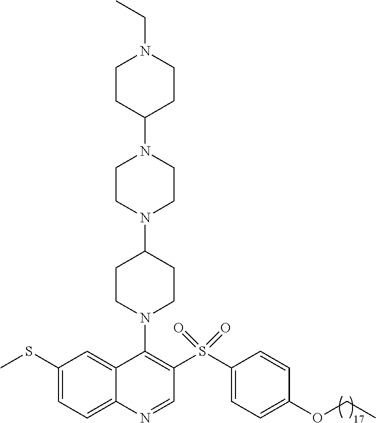 |
| 508 | 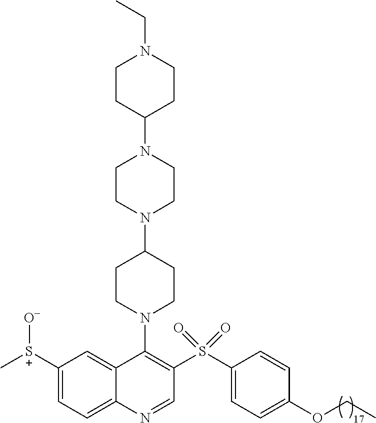 |
| 509 | 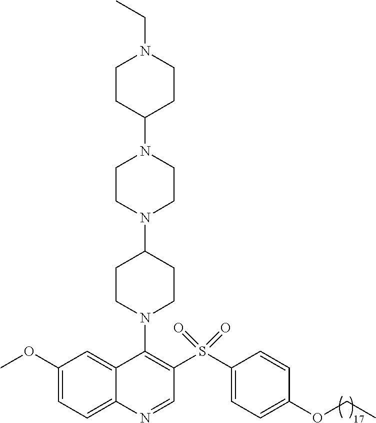 |
| 510 | 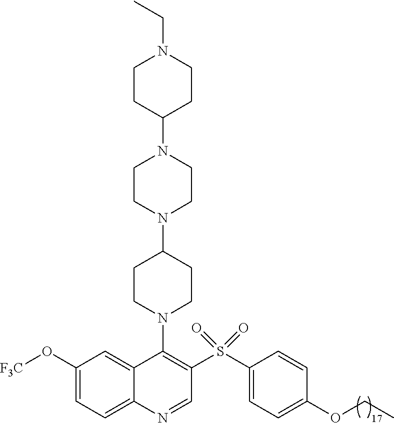 |
| 511 | 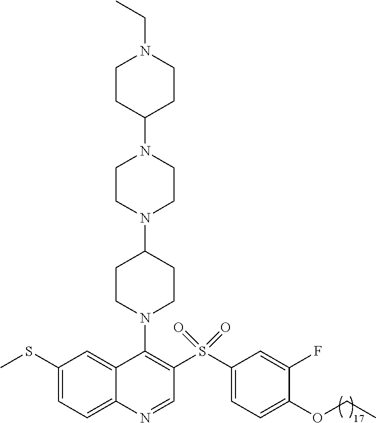 |
| 512 | 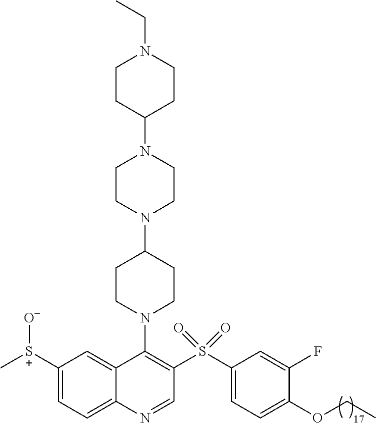 |
| 513 | 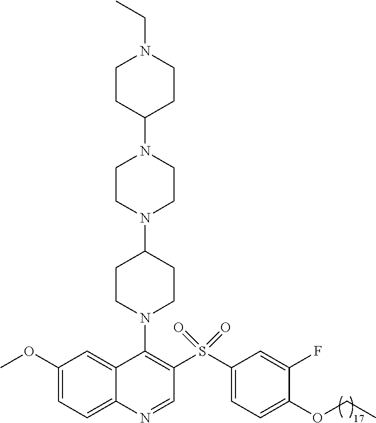 |
| 514 | 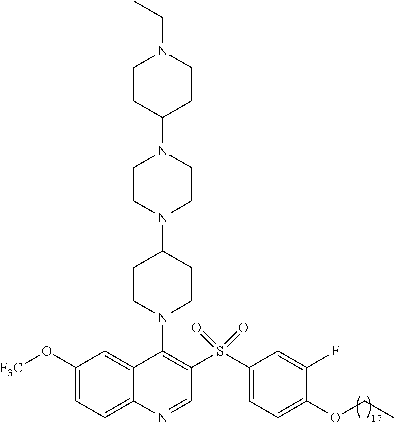 |
| 515 | 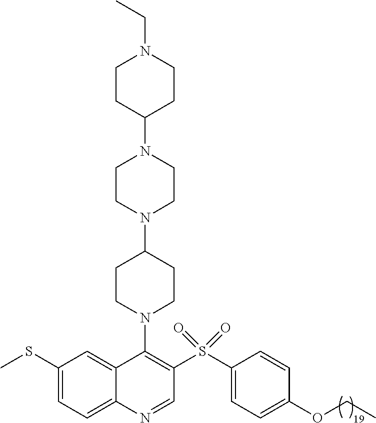 |
| 516 | 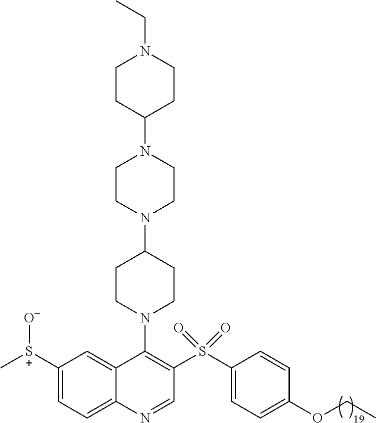 |
| 517 | 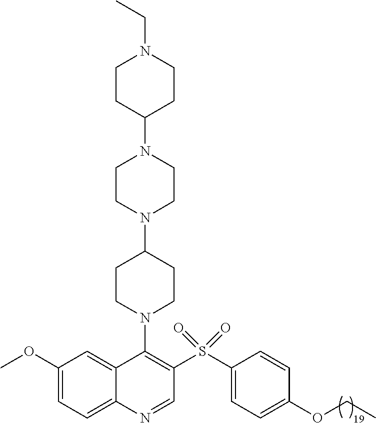 |
| 518 | 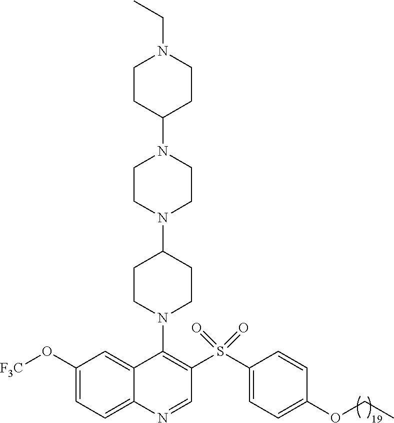 |
| 519 | 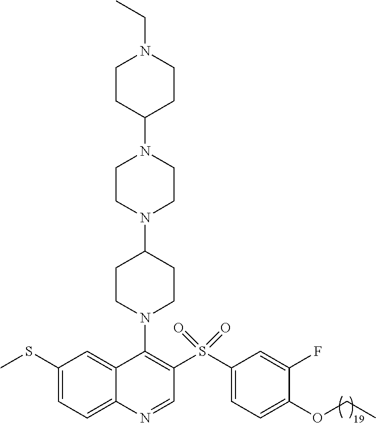 |
| 520 | 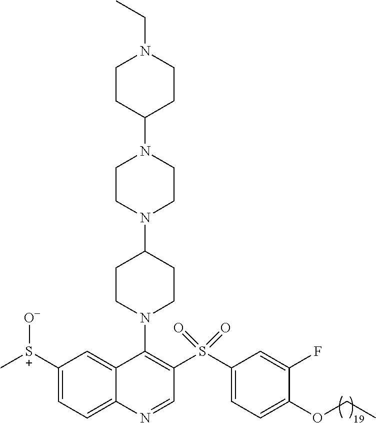 |
| 521 | 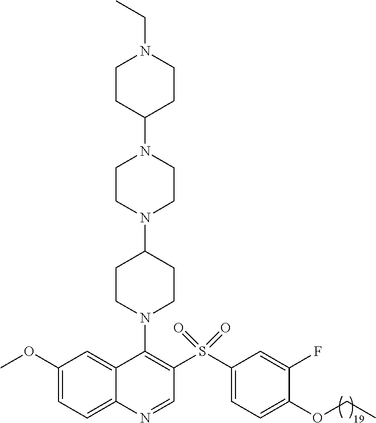 |
| 522 | 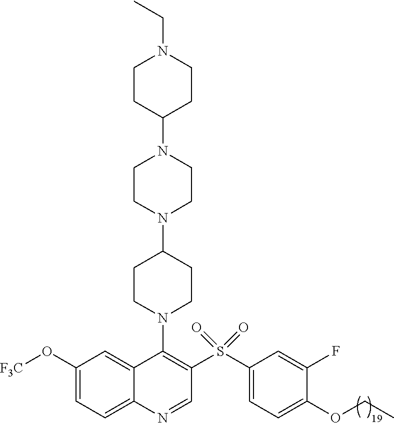 |
| 523 | 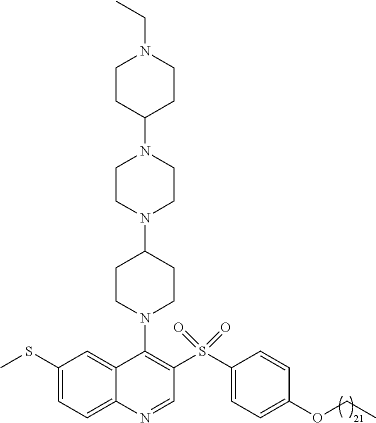 |
| 524 | 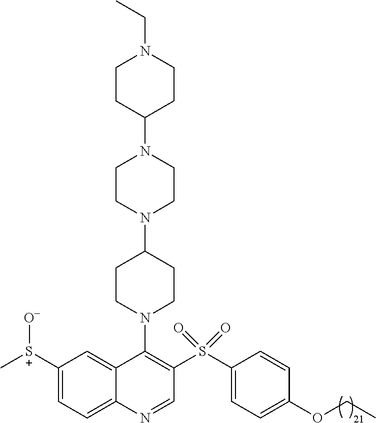 |
| 525 | 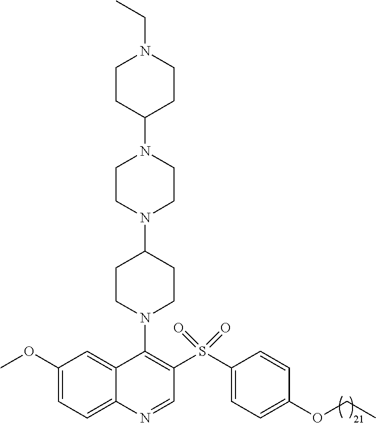 |
| 526 | 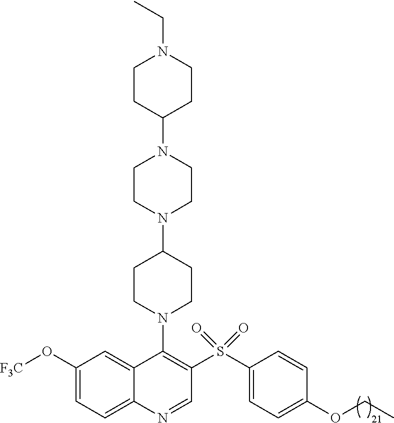 |
| 527 | 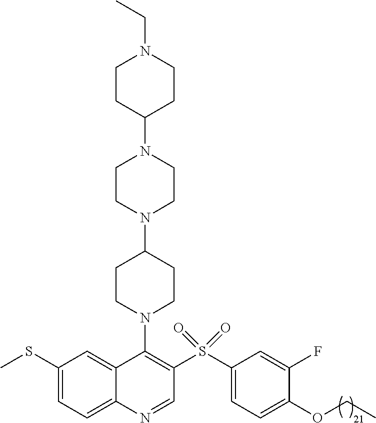 |
| 528 | 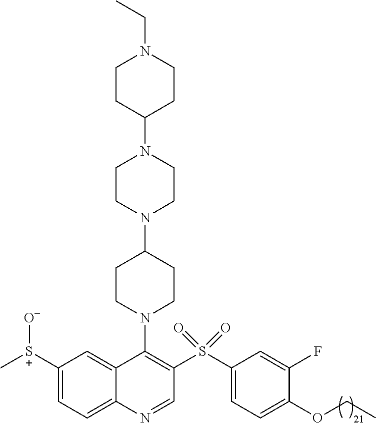 |
| 529 | 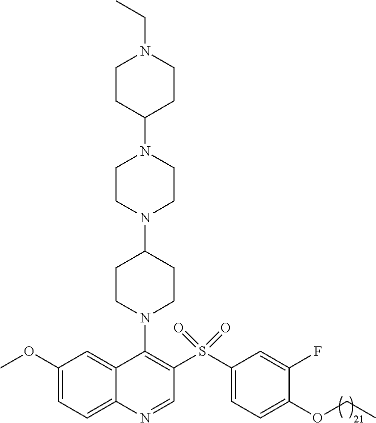 |
| 530 | 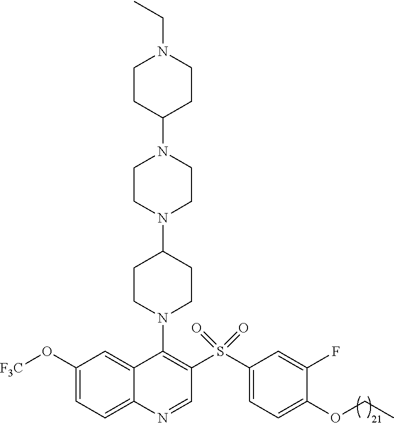 |
| 531 | 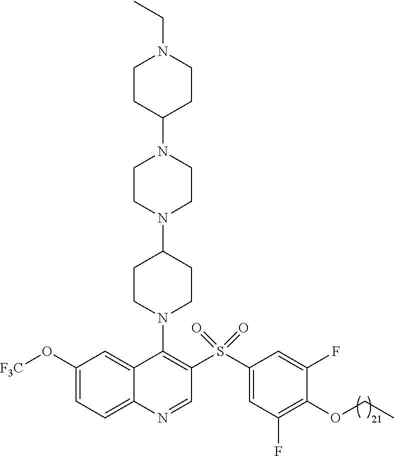 |
| 532 | 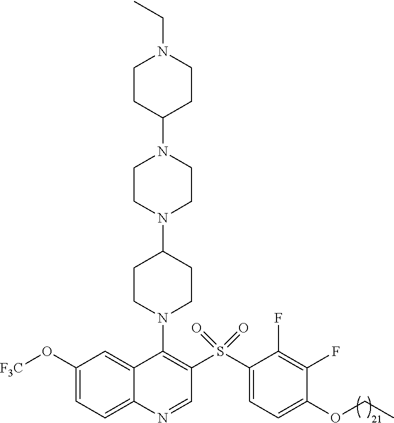 |
| 533 | 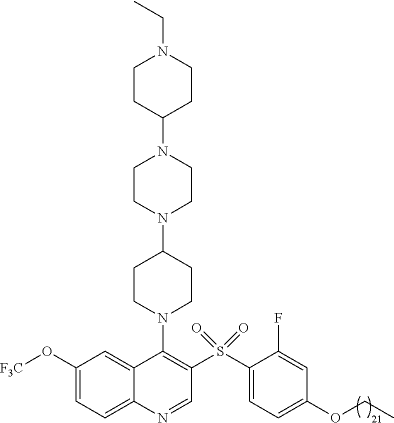 |
| 534 | 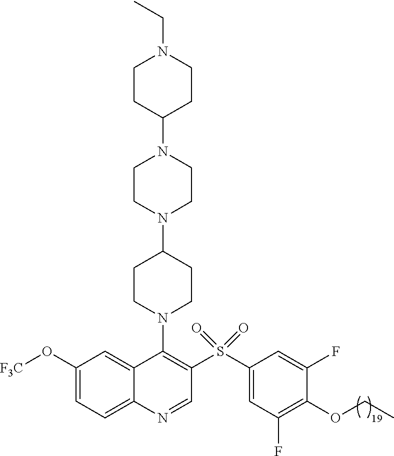 |
| 535 | 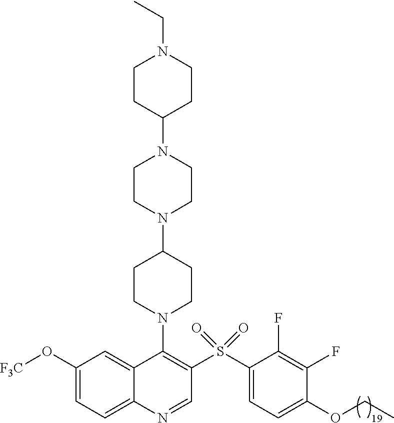 |
| 536 | 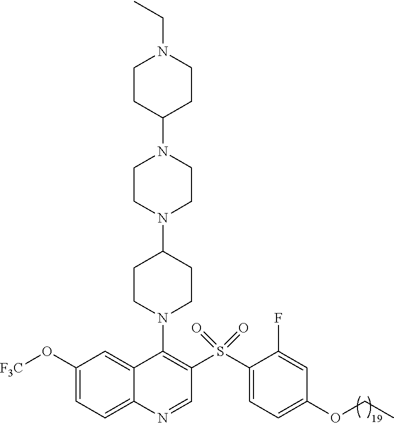 |
| 537 | 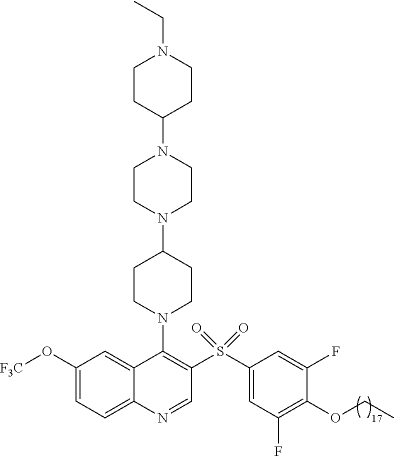 |
| 538 | 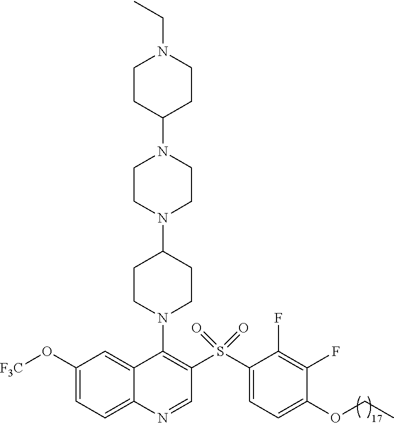 |
| 539 | 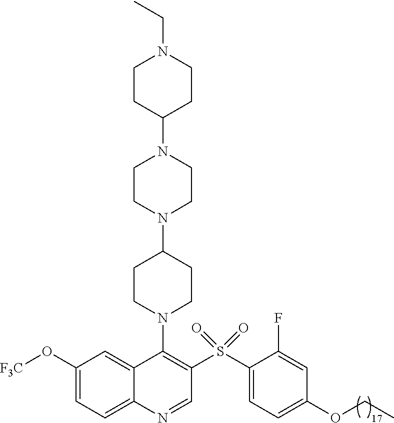 |
| 540 | 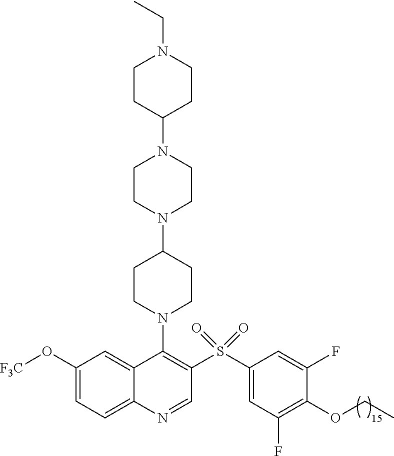 |
| 541 | 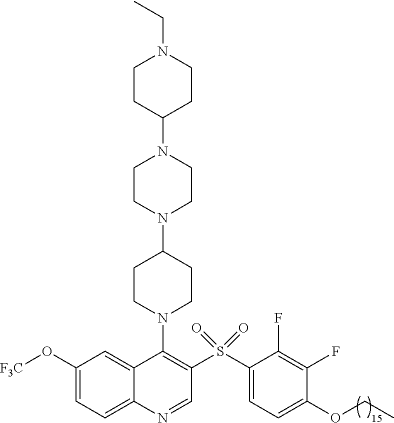 |
| 542 | 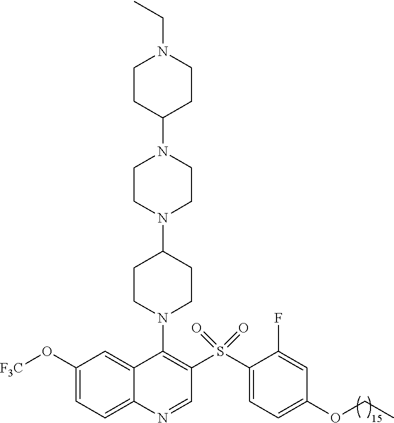 |

Compounds shown in Table 3 may be synthesized according to the procedures described in US. Pat. 62/916,983 or PCT/US20/55979, each of which are incorporated by reference for all purposes.

### IV. Antibodies

Disclosed herein are methods of modulating the activity of a PLXDC1 and/or PLXDC2 receptor using an antibody (e.g., an antibody that activates the PLXDC1 and/or PLXDC2 receptor, such as an antibody disclosed herein, such as an antibody that binds to the extracellular domain of a receptor described herein, such as AA02, AA03, or AA94, or those in Table 6). In some embodiments, the antibody is an anti-PLXDC1 and/or anti-PLXDC2 antibody. In some embodiments, the antibody is specific for a PLXDC1 and/or PLXDC2 protein (e.g., an extracellular domain of PLXDC1 and/or PLXDC2).

A. Antibodies

The term “antibody” refers to an intact immunoglobulin of any isotype, or a fragment thereof that can compete with the intact antibody for specific binding to the target antigen, and includes chimeric, humanized, fully human, and bispecific antibodies. As used herein, the terms “antibody” or “immunoglobulin” are used interchangeably and refer to any of several classes of structurally related proteins that function as part of the immune response of an animal, including IgG, IgD, IgE, IgA, IgM, and related proteins, as well as polypeptides comprising antibody CDR domains that retain antigen-binding activity.

The term “antigen” refers to a molecule or a portion of a molecule capable of being bound by a selective binding agent, such as an antibody. An antigen may possess one or more epitopes that are capable of interacting with different antibodies.

The term “epitope” includes any region or portion of molecule capable eliciting an immune response by binding to an immunoglobulin or to a T-cell receptor. Epitope determinants may include chemically active surface groups such as amino acids, sugar side chains, phosphoryl or sulfonyl groups, and may have specific three-dimensional structural characteristics and/or specific charge characteristics. Generally, antibodies specific for a particular target antigen will preferentially recognize an epitope on the target antigen within a complex mixture.

The epitope regions of a given polypeptide can be identified using many different epitope mapping techniques are well known in the art, including: x-ray crystallography, nuclear magnetic resonance spectroscopy, site-directed mutagenesis mapping, protein display arrays, see, e.g., Epitope Mapping Protocols, (Johan Rockberg and Johan Nilvebrant, Ed., 2018) Humana Press, New York, N.Y. Such techniques are known in the art and described in, e.g., U.S. Pat. No. 4,708,871; Geysen et al. Proc. Natl. Acad. Sci. USA 81:3998-4002 (1984); Geysen et al. Proc. Natl. Acad. Sci. USA 82:178-182 (1985); Geysen et al. Molec. Immunol. 23:709-715 (1986). Additionally, antigenic regions of proteins can also be predicted and identified using standard antigenicity and hydropathy plots.

The term “immunogenic sequence” means a molecule that includes an amino acid sequence of at least one epitope such that the molecule is capable of stimulating the production of antibodies in an appropriate host. The term “immunogenic composition” means a composition that comprises at least one immunogenic molecule (e.g., an antigen or carbohydrate).

An intact antibody is generally composed of two full-length heavy chains and two full-length light chains, but in some instances may include fewer chains, such as antibodies naturally occurring in camelids that may comprise only heavy chains. Antibodies as disclosed herein may be derived solely from a single source or may be “chimeric,” that is, different portions of the antibody may be derived from two different antibodies. For example, the variable or CDR regions may be derived from a rat or murine source, while the constant region is derived from a different animal source, such as a human. The antibodies or binding fragments may be produced in hybridomas, by recombinant DNA techniques, or by enzymatic or chemical cleavage of intact antibodies. Unless otherwise indicated, the term “antibody” includes derivatives, variants, fragments, and muteins thereof, examples of which are described below (Sela-Culang et al., Front Immunol. 2013; 4: 302; 2013).

The term “light chain” includes a full-length light chain and fragments thereof having sufficient variable region sequence to confer binding specificity. A full-length light chain has a molecular weight of around 25,000 Daltons and includes a variable region domain (abbreviated herein as VL), and a constant region domain (abbreviated herein as CL). There are two classifications of light chains, identified as kappa (X) and lambda (λ). The term “VL fragment” means a fragment of the light chain of a monoclonal antibody that includes all or part of the light chain variable region, including CDRs. A VL fragment can further include light chain constant region sequences. The variable region domain of the light chain is at the amino-terminus of the polypeptide.

The term “heavy chain” includes a full-length heavy chain and fragments thereof having sufficient variable region sequence to confer binding specificity. A full-length heavy chain has a molecular weight of around 50,000 Daltons and includes a variable region domain (abbreviated herein as VH), and three constant region domains (abbreviated herein as CH₁, CH₂, and CH₃). The term “VH fragment” means a fragment of the heavy chain of a monoclonal antibody that includes all or part of the heavy chain variable region, including CDRs. A VH fragment can further include heavy chain constant region sequences. The number of heavy chain constant region domains will depend on the isotype. The VH domain is at the amino-terminus of the polypeptide, and the CH domains are at the carboxy-terminus, with the CH₃ being closest to the —COOH end. The isotype of an antibody can be IgM, IgD, IgG, IgA, or IgE and is defined by the heavy chains present of which there are five classifications: mu (μ), delta (d), gamma (γ), alpha (α), or epsilon (ε) chains, respectively. IgG has several subtypes, including, but not limited to, IgG1, IgG2, IgG3, and IgG4. IgM subtypes include IgM1 and IgM2. IgA subtypes include IgA1 and IgA2.

Antibodies can be whole immunoglobulins of any isotype or classification, chimeric antibodies, or hybrid antibodies with specificity to two or more antigens. They may also be fragments (e.g., F(ab′)2, Fab′, Fab, Fv, and the like), including hybrid fragments. An immunoglobulin also includes natural, synthetic, or genetically engineered proteins that act like an antibody by binding to specific antigens to form a complex. The term antibody includes genetically engineered or otherwise modified forms of immunoglobulins, such as the following:

The term “monomer” means an antibody containing only one Ig unit. Monomers are the basic functional units of antibodies. The term “dimer” means an antibody containing two Ig units attached to one another via constant domains of the antibody heavy chains (the Fc, or fragment crystallizable, region). The complex may be stabilized by a joining (J) chain protein. The term “multimer” means an antibody containing more than two Ig units attached to one another via constant domains of the antibody heavy chains (the Fc region). The complex may be stabilized by a joining (J) chain protein.

The term “bivalent antibody” means an antibody that comprises two antigen-binding sites. The two binding sites may have the same antigen specificities or they may be bi-specific, meaning the two antigen-binding sites have different antigen specificities.

Bispecific antibodies are a class of antibodies that have two paratopes with different binding sites for two or more distinct epitopes. In some embodiments, bispecific antibodies can be biparatopic, wherein a bispecific antibody may specifically recognize a different epitope from the same antigen. In some embodiments, bispecific antibodies can be constructed from a pair of different single domain antibodies termed “nanobodies”. Single domain antibodies are sourced and modified from cartilaginous fish and camelids. Nanobodies can be joined together by a linker using techniques typical to a person skilled in the art; such methods for selection and joining of nanobodies are described in PCT Publication No. WO2015044386A1, No. WO2010037838A2, and Bever et al., Anal Chem. 86:7875-7882 (2014), each of which are specifically incorporated herein by reference in their entirety.

Bispecific antibodies can be constructed as: a whole IgG, Fab′2, Fab′PEG, a diabody, or alternatively as scFv. Diabodies and scFvs can be constructed without an Fc region, using only variable domains, potentially reducing the effects of anti-idiotypic reaction. Bispecific antibodies may be produced by a variety of methods including, but not limited to, fusion of hybridomas or linking of Fab′ fragments. See, e.g., Songsivilai and Lachmann, Clin. Exp. Immunol. 79:315-321 (1990); Kostelny et al., J. Immunol. 148:1547-1553 (1992), each of which are specifically incorporated by reference in their entirety.

In certain aspects, the antigen-binding domain may be multispecific or heterospecific by multimerizing with VH and VL region pairs that bind a different antigen. For example, the antibody may bind to, or interact with, (a) a cell surface antigen, (b) an Fc receptor on the surface of an effector cell, or (c) at least one other component. Accordingly, aspects may include, but are not limited to, bispecific, trispecific, tetraspecific, and other multispecific antibodies or antigen-binding fragments thereof that are directed to epitopes and to other targets, such as Fc receptors on effector cells.

In some embodiments, multispecific antibodies can be used and directly linked via a short flexible polypeptide chain, using routine methods known in the art. One such example is diabodies that are bivalent, bispecific antibodies in which the VH and VL domains are expressed on a single polypeptide chain, and utilize a linker that is too short to allow for pairing between domains on the same chain, thereby forcing the domains to pair with complementary domains of another chain creating two antigen binding sites. The linker functionality is applicable for embodiments of triabodies, tetrabodies, and higher order antibody multimers. (see, e.g., Hollinger et al., Proc Natl. Acad. Sci. USA 90:6444-6448 (1993); Polijak et al., Structure 2:1121-1123 (1994); Todorovska et al., J. Immunol. Methods 248:47-66 (2001)).

Bispecific diabodies, as opposed to bispecific whole antibodies, may also be advantageous because they can be readily constructed and expressed in E. coli. Diabodies (and other polypeptides such as antibody fragments) of appropriate binding specificities can be readily selected using phage display (WO94/13804) from libraries. If one arm of the diabody is kept constant, for instance, with a specificity directed against a protein, then a library can be made where the other arm is varied and an antibody of appropriate specificity selected. Bispecific whole antibodies may be made by alternative engineering methods as described in Ridgeway et al., (Protein Eng., 9:616-621, 1996) and Krah et al., (N Biotechnol. 39:167-173, 2017), each of which is hereby incorporated by reference in their entirety.

Heteroconjugate antibodies are composed of two covalently linked monoclonal antibodies with different specificities. See, e.g., U.S. Pat. No. 6,010,902, incorporated herein by reference in its entirety.

The part of the Fv fragment of an antibody molecule that binds with high specificity to the epitope of the antigen is referred to herein as the “paratope.” The paratope consists of the amino acid residues that make contact with the epitope of an antigen to facilitate antigen recognition. Each of the two Fv fragments of an antibody is composed of the two variable domains, V_{H} and V_{L}, in dimerized configuration. The primary structure of each of the variable domains includes three hypervariable loops separated by, and flanked by, Framework Regions (FR). The hypervariable loops are the regions of highest primary sequences variability among the antibody molecules from any mammal. The term hypervariable loop is sometimes used interchangeably with the term “Complementarity Determining Region (CDR).” The length of the hypervariable loops (or CDRs) varies between antibody molecules. The framework regions of all antibody molecules from a given mammal have high primary sequence similarity/consensus. The consensus of framework regions can be used by one skilled in the art to identify both the framework regions and the hypervariable loops (or CDRs) which are interspersed among the framework regions. The hypervariable loops are given identifying names which distinguish their position within the polypeptide, and on which domain they occur. CDRs in the V_{L} domain are identified as L1, L2, and L3, with L1 occurring at the most distal end and L3 occurring closest to the C_{L} domain. The CDRs may also be given the names CDR-1, CDR-2, and CDR-3. The L3 (CDR-3) is generally the region of highest variability among all antibody molecules produced by a given organism. The CDRs are regions of the polypeptide chain arranged linearly in the primary structure, and separated from each other by Framework Regions. The amino terminal (N-terminal) end of the V_{L} chain is named FR1. The region identified as FR2 occurs between L1 and L2 hypervariable loops. FR3 occurs between L2 and L3 hypervariable loops, and the FR4 region is closest to the C_{L} domain. This structure and nomenclature is repeated for the V_{H} chain, which includes three CDRs identified as H1, H2 and H3. The majority of amino acid residues in the variable domains, or Fv fragments (V_{H} and V_{L}), are part of the framework regions (approximately 85%). The three dimensional, or tertiary, structure of an antibody molecule is such that the framework regions are more internal to the molecule and provide the majority of the structure, with the CDRs on the external surface of the molecule.

Several methods have been developed and can be used by one skilled in the art to identify the exact amino acids that constitute each of these regions. This can be done using any of a number of multiple sequence alignment methods and algorithms, which identify the conserved amino acid residues that make up the framework regions, therefore identifying the CDRs that may vary in length but are located between framework regions. Three commonly used methods have been developed for identification of the CDRs of antibodies: Kabat (as described in T. T. Wu and E. A. Kabat, “AN ANALYSIS OF THE SEQUENCES OF THE VARIABLE REGIONS OF BENCE JONES PROTEINS AND MYELOMA LIGHT CHAINS AND THEIR IMPLICATIONS FOR ANTIBODY COMPLEMENTARITY,” J Exp Med, vol. 132, no. 2, pp. 211-250, August 1970); Chothia (as described in C. Chothia et al., “Conformations of immunoglobulin hypervariable regions,” Nature, vol. 342, no. 6252, pp. 877-883, December 1989); and IMGT (as described in M.-P. Lefranc et al., “IMGT unique numbering for immunoglobulin and T cell receptor variable domains and Ig superfamily V-like domains,” Developmental & Comparative Immunology, vol. 27, no. 1, pp. 55-77, January 2003). These methods each include unique numbering systems for the identification of the amino acid residues that constitute the variable regions. In most antibody molecules, the amino acid residues that actually contact the epitope of the antigen occur in the CDRs, although in some cases, residues within the framework regions contribute to antigen binding.

One skilled in the art can use any of several methods to determine the paratope of an antibody. These methods include: 1) Computational predictions of the tertiary structure of the antibody/epitope binding interactions based on the chemical nature of the amino acid sequence of the antibody variable region and composition of the epitope; 2) Hydrogen-deuterium exchange and mass spectroscopy; 3) Polypeptide fragmentation and peptide mapping approaches in which one generates multiple overlapping peptide fragments from the full length of the polypeptide and evaluates the binding affinity of these peptides for the epitope; 4) Antibody Phage Display Library analysis in which the antibody Fab fragment encoding genes of the mammal are expressed by bacteriophage in such a way as to be incorporated into the coat of the phage. This population of Fab expressing phage are then allowed to interact with the antigen which has been immobilized or may be expressed in by a different exogenous expression system. Non-binding Fab fragments are washed away, thereby leaving only the specific binding Fab fragments attached to the antigen. The binding Fab fragments can be readily isolated and the genes which encode them determined. This approach can also be used for smaller regions of the Fab fragment including Fv fragments or specific V_{H} and V_{L} domains as appropriate.

In certain aspects, affinity matured antibodies are enhanced with one or more modifications in one or more CDRs thereof that result in an improvement in the affinity of the antibody for a target antigen as compared to a parent antibody that does not possess those alteration(s). Certain affinity matured antibodies will have nanomolar or picomolar affinities for the target antigen. Affinity matured antibodies are produced by procedures known in the art, e.g., Marks et al., Bio/Technology 10:779 (1992) describes affinity maturation by VH and VL domain shuffling, random mutagenesis of CDR and/or framework residues employed in phage display is described by Rajpal et al., PNAS. 24: 8466-8471 (2005) and Thie et al., Methods Mol Biol. 525:309-22 (2009) in conjugation with computation methods as demonstrated in Tiller et al., Front. Immunol. 8:986 (2017).

Chimeric immunoglobulins are the products of fused genes derived from different species; “humanized” chimeras generally have the framework region (FR) from human immunoglobulins and one or more CDRs are from a non-human source.

In certain aspects, portions of the heavy and/or light chain are identical or homologous to corresponding sequences from another particular species or belonging to a particular antibody class or subclass, while the remainder of the chain(s) is identical or homologous to corresponding sequences in antibodies derived from another species or belonging to another antibody class or subclass, as well as fragments of such antibodies, so long as they exhibit the desired biological activity. U.S. Pat. No. 4,816,567; and Morrison et al., Proc. Natl. Acad. Sci. USA 81:6851 (1984). For methods relating to chimeric antibodies, see, e.g., U.S. Pat. No. 4,816,567; and Morrison et al., Proc. Natl. Acad. Sci. USA 81:6851-6855 (1985), each of which are specifically incorporated herein by reference in their entirety. CDR grafting is described, for example, in U.S. Pat. Nos. 6,180,370, 5,693,762, 5,693,761, 5,585,089, and 5,530,101, which are all hereby incorporated by reference for all purposes.

In some embodiments, minimizing the antibody polypeptide sequence from the non-human species optimizes chimeric antibody function and reduces immunogenicity. Specific amino acid residues from non-antigen recognizing regions of the non-human antibody are modified to be homologous to corresponding residues in a human antibody or isotype. One example is the “CDR-grafted” antibody, in which an antibody comprises one or more CDRs from a particular species or belonging to a specific antibody class or subclass, while the remainder of the antibody chain(s) is identical or homologous to a corresponding sequence in antibodies derived from another species or belonging to another antibody class or subclass. For use in humans, the V region composed of CDR1, CDR2, and partial CDR3 for both the light and heavy chain variance region from a non-human immunoglobulin, are grafted with a human antibody framework region, replacing the naturally occurring antigen receptors of the human antibody with the non-human CDRs. In some instances, corresponding non-human residues replace framework region residues of the human immunoglobulin. Furthermore, humanized antibodies may comprise residues that are not found in the recipient antibody or in the donor antibody to further refine performance. The humanized antibody may also comprise at least a portion of an immunoglobulin constant region (Fc), typically that of a human immunoglobulin. See, e.g., Jones et al., Nature 321:522 (1986); Riechmann et at, Nature 332:323 (1988); Presta, Curr. Op. Struct. Biol. 2:593 (1992); Vaswani and Hamilton, Ann. Allergy, Asthma and Immunol. 1:105 (1998); Harris, Biochem. Soc. Transactions 23; 1035 (1995); Hurle and Gross, Curr. Op. Biotech. 5:428 (1994); Verhoeyen et al., Science 239:1534-36 (1988).

Intrabodies are intracellularly localized immunoglobulins that bind to intracellular antigens as opposed to secreted antibodies, which bind antigens in the extracellular space.

Polyclonal antibody preparations typically include different antibodies against different determinants (epitopes). In order to produce polyclonal antibodies, a host, such as a rabbit or goat, is immunized with the antigen or antigen fragment, generally with an adjuvant and, if necessary, coupled to a carrier. Antibodies to the antigen are subsequently collected from the sera of the host. The polyclonal antibody can be affinity purified against the antigen rendering it monospecific.

Monoclonal antibodies or “mAb” refer to an antibody obtained from a population of homogeneous antibodies from an exclusive parental cell, e.g., the population is identical except for naturally occurring mutations that may be present in minor amounts. Each monoclonal antibody is directed against a single antigenic determinant.

Certain aspects relate to antibody fragments, such as antibody fragments that bind to PLXDC1 or PLXDC2. The term functional antibody fragment includes antigen-binding fragments of an antibody that retain the ability to specifically bind to an antigen. These fragments are constituted of various arrangements of the variable region heavy chain (VH) and/or light chain (VL); and in some embodiments, include constant region heavy chain 1 (CH₁) and light chain (CL). In some embodiments, they lack the Fc region constituted of heavy chain 2 (CH₂) and 3 (CH₃) domains. Embodiments of antigen binding fragments and the modifications thereof may include: (i) the Fab fragment type constituted with the VL, VH, CL, and CH₁ domains; (ii) the Fd fragment type constituted with the VH and CH₁ domains; (iii) the Fv fragment type constituted with the VH and VL domains; (iv) the single domain fragment type, dAb, (Ward, 1989; McCafferty et al., 1990; Holt et al., 2003) constituted with a single VH or VL domain; (v) isolated complementarity determining region (CDR) regions. Such terms are described, for example, in Harlow and Lane, Antibodies: A Laboratory Manual, Cold Spring Harbor Laboratory, N Y (1989); Molec. Biology and Biotechnology: A Comprehensive Desk Reference (Myers, R. A. (ed.), New York: VCH Publisher, Inc.); Huston et al., Cell Biophysics, 22:189-224 (1993); Pluckthun and Skerra, Meth. Enzymol., 178:497-515 (1989) and in Day, E. D., Advanced Immunochemistry, 2d ed., Wiley-Liss, Inc. New York, N.Y. (1990); Antibodies, 4:259-277 (2015). The citations in this paragraph are all incorporated by reference.

Antigen-binding fragments also include fragments of an antibody that retain exactly, at least, or at most 1, 2, or 3 complementarity determining regions (CDRs) from a light chain variable region. Fusions of CDR-containing sequences to an Fc region (or a CH₂ or CH₃ region thereof) are included within the scope of this definition including, for example, scFv fused, directly or indirectly, to an Fc region are included herein.

The term Fab fragment means a monovalent antigen-binding fragment of an antibody containing the VL, VH, CL and CH₁ domains. The term Fab′ fragment means a monovalent antigen-binding fragment of a monoclonal antibody that is larger than a Fab fragment. For example, a Fab′ fragment includes the VL, VH, CL and CH₁ domains and all or part of the hinge region. The term F(ab′)2 fragment means a bivalent antigen-binding fragment of a monoclonal antibody comprising two Fab′ fragments linked by a disulfide bridge at the hinge region. An F(ab′)2 fragment includes, for example, all or part of the two VH and VL domains, and can further include all or part of the two CL and CH₁ domains.

The term Fd fragment means a fragment of the heavy chain of a monoclonal antibody, which includes all or part of the VH, including the CDRs. An Fd fragment can further include CH₁ region sequences.

The term Fv fragment means a monovalent antigen-binding fragment of a monoclonal antibody, including all or part of the VL and VH, and absent of the CL and CH₁ domains. The VL and VH include, for example, the CDRs. Single-chain antibodies (sFv or scFv) are Fv molecules in which the VL and VH regions have been connected by a flexible linker to form a single polypeptide chain, which forms an antigen-binding fragment. Single chain antibodies are discussed in detail in International Patent Application Publication No. WO 88/01649 and U.S. Pat. Nos. 4,946,778 and 5,260,203, the disclosures of which are herein incorporated by reference. The term (scFv)2 means bivalent or bispecific sFv polypeptide chains that include oligomerization domains at their C-termini, separated from the sFv by a hinge region (Pack et al. 1992). The oligomerization domain comprises self-associating a-helices, e.g., leucine zippers, which can be further stabilized by additional disulfide bonds. (scFv)2 fragments are also known as “miniantibodies” or “minibodies.”

A single domain antibody is an antigen-binding fragment containing only a VH or the VL domain. In some instances, two or more VH regions are covalently joined with a peptide linker to create a bivalent domain antibody. The two VH regions of a bivalent domain antibody may target the same or different antigens.

An Fc region contains two heavy chain fragments comprising the CH₂ and CH₃ domains of an antibody. The two heavy chain fragments are held together by two or more disulfide bonds and by hydrophobic interactions of the CH₃ domains. The term “Fc polypeptide” as used herein includes native and mutein forms of polypeptides derived from the Fc region of an antibody. Truncated forms of such polypeptides containing the hinge region that promotes dimerization are included.

Antigen-binding peptide scaffolds, such as complementarity-determining regions (CDRs), are used to generate protein-binding molecules in accordance with the embodiments. Generally, a person skilled in the art can determine the type of protein scaffold on which to graft at least one of the CDRs. It is known that scaffolds, optimally, must meet a number of criteria such as: good phylogenetic conservation; known three-dimensional structure; small size; few or no post-transcriptional modifications; and/or be easy to produce, express, and purify. Skerra, J Mol Recognit, 13:167-87 (2000).

The protein scaffolds can be sourced from, but not limited to: fibronectin type III FN3 domain (known as “monobodies”), fibronectin type III domain 10, lipocalin, anticalin, Z— domain of protein A of Staphylococcus aureus, thioredoxin A or proteins with a repeated motif such as the “ankyrin repeat”, the “armadillo repeat”, the “leucine-rich repeat” and the “tetratricopeptide repeat”. Such proteins are described in US Patent Publication Nos. 2010/0285564, 2006/0058510, 2006/0088908, 2005/0106660, and PCT Publication No. WO2006/056464, each of which are specifically incorporated herein by reference in their entirety. Scaffolds derived from toxins from scorpions, insects, plants, mollusks, etc., and the protein inhibiters of neuronal NO synthase (PIN) may also be used.

The term “selective binding agent” refers to a molecule that binds to an antigen. Non-limiting examples include antibodies, antigen-binding fragments, scFv, Fab, Fab′, F(ab′)2, single chain antibodies, peptides, peptide fragments and proteins.

The term “binding” refers to a direct association between two molecules, due to, for example, covalent, electrostatic, hydrophobic, and ionic and/or hydrogen-bond interactions, including interactions such as salt bridges and water bridges “Immunologically reactive” means that the selective binding agent or antibody of interest will bind with antigens present in a biological sample. The term “immune complex” refers the combination formed when an antibody or selective binding agent binds to an epitope on an antigen.

The term “affinity” refers the strength with which an antibody or selective binding agent binds an epitope. In antibody binding reactions, this is expressed as the affinity constant (Ka or ka sometimes referred to as the association constant) for any given antibody or selective binding agent. Affinity is measured as a comparison of the binding strength of the antibody to its antigen relative to the binding strength of the antibody to an unrelated amino acid sequence. Affinity can be expressed as, for example, 20-fold greater binding ability of the antibody to its antigen then to an unrelated amino acid sequence. As used herein, the term “avidity” refers to the resistance of a complex of two or more agents to dissociation after dilution. The terms “immunoreactive” and “preferentially binds” are used interchangeably herein with respect to antibodies and/or selective binding agent.

There are several experimental methods that can be used by one skilled in the art to evaluate the binding affinity of any given antibody or selective binding agent for its antigen. This is generally done by measuring the equilibrium dissociation constant (KD or Kd), using the equation KD=koff/kon=[A] [B]/[AB]. The term koff is the rate of dissociation between the antibody and antigen per unit time, and is related to the concentration of antibody and antigen present in solution in the unbound form at equilibrium. The term kon is the rate of antibody and antigen association per unit time, and is related to the concentration of the bound antigen-antibody complex at equilibrium. The units used for measuring the KD are mol/L (molarity, or M), or concentration. The Ka of an antibody is the opposite of the KD, and is determined by the equation Ka=1/KD. Examples of some experimental methods that can be used to determine the KD value are: enzyme-linked immunosorbent assays (ELISA), isothermal titration calorimetry (ITC), fluorescence anisotropy, surface plasmon resonance (SPR), and affinity capillary electrophoresis (ACE). The affinity constant (Ka) of an antibody is the opposite of the KD, and is determined by the equation Ka=1/KD.

Antibodies deemed useful in certain embodiments may have an affinity constant (Ka) of about, at least about, or at most about 10⁶, 10⁷, 10⁸, 10⁹, or 10¹⁰ M or any range derivable therein. Similarly, in some embodiments, antibodies may have a dissociation constant of about, at least about or at most about 10⁻⁶, 10⁻⁷, 10⁻⁸, 10⁻⁹, 10⁻¹⁰ M, or any range derivable therein. These values are reported for antibodies discussed herein and the same assay may be used to evaluate the binding properties of such antibodies. An antibody of the disclosure is said to “specifically bind” its target antigen when the dissociation constant (KD) is ≤10⁻⁸ M. The antibody specifically binds antigen with “high affinity” when the KD is ≤5×10⁻⁹ M, and with “very high affinity” when the KD is ≤5×10⁻¹⁰ M.

The epitope of an antigen is the specific region of the antigen for which an antibody has binding affinity. In the case of protein or polypeptide antigens, the epitope is the specific residues (or specified amino acids or protein segment) that the antibody binds with high affinity. An antibody does not necessarily contact every residue within the protein. Nor does every single amino acid substitution or deletion within a protein necessarily affect binding affinity. For purposes of this specification and the accompanying claims, the terms “epitope” and “antigenic determinant” are used interchangeably to refer to the site on an antigen to which B and/or T cells respond or recognize. Polypeptide epitopes can be formed from both contiguous amino acids and noncontiguous amino acids juxtaposed by tertiary folding of a polypeptide. An epitope typically includes at least 3, and typically 5-10 amino acids in a unique spatial conformation.

Epitope specificity of an antibody can be determined in a variety of ways. One approach, for example, involves testing a collection of overlapping peptides of about 15 amino acids spanning the full sequence of the protein and differing in increments of a small number of amino acids (e.g., 3 to 30 amino acids). The peptides are immobilized in separate wells of a microtiter dish Immobilization can be accomplished, for example, by biotinylating one terminus of the peptides. This process may affect the antibody affinity for the epitope, therefore different samples of the same peptide can be biotinylated at the N and C terminus and immobilized in separate wells for the purposes of comparison. This is useful for identifying end-specific antibodies. Optionally, additional peptides can be included terminating at a particular amino acid of interest. This approach is useful for identifying end-specific antibodies to internal fragments. An antibody or antigen-binding fragment is screened for binding to each of the various peptides. The epitope is defined as a segment of amino acids that is common to all peptides to which the antibody shows high affinity binding.

It is understood that the antibodies of the present disclosure may be modified, such that they are substantially identical to the antibody polypeptide sequences, or fragments thereof, and still bind the epitopes of the present disclosure. Polypeptide sequences are “substantially identical” when optimally aligned using such programs as Clustal Omega, IGBLAST, GAP or BESTFIT using default gap weights, they share at least 80% sequence identity, at least 90% sequence identity, at least 95% sequence identity, at least 96% sequence identity, at least 97% sequence identity, at least 98% sequence identity, or at least 99% sequence identity or any range therein.

As discussed herein, minor variations in the amino acid sequences of antibodies or antigen-binding regions thereof are contemplated as being encompassed by the present disclosure, providing that the variations in the amino acid sequence maintain at least 75%, more preferably at least 80%, at least 90%, at least 95%, at least 96%, at least 97%, at least 98% and most preferably at least 99% sequence identity. In particular, conservative amino acid replacements are contemplated.

Conservative replacements are those that take place within a family of amino acids that are related in their side chains. Genetically encoded amino acids are generally divided into families based on the chemical nature of the side chain; e.g., acidic (aspartate, glutamate), basic (lysine, arginine, histidine), nonpolar (alanine, valine, leucine, isoleucine, proline, phenylalanine, methionine, tryptophan), and uncharged polar (glycine, asparagine, glutamine, cysteine, serine, threonine, tyrosine). For example, it is reasonable to expect that an isolated replacement of a leucine moiety with an isoleucine or valine moiety, or a similar replacement of an amino acid with a structurally related amino acid in the same family, will not have a major effect on the binding or properties of the resulting molecule, especially if the replacement does not involve an amino acid within a framework site. Whether an amino acid change results in a functional peptide can readily be determined by assaying the specific activity of the polypeptide derivative. Standard ELISA, Surface Plasmon Resonance (SPR), or other antibody binding assays can be performed by one skilled in the art to make a quantitative comparison of antigen binging affinity between the unmodified antibody and any polypeptide derivatives with conservative substitutions generated through any of several methods available to one skilled in the art.

Fragments or analogs of antibodies or immunoglobulin molecules can be readily prepared by those skilled in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Standard methods to identify protein sequences that fold into a known three-dimensional structure are available to those skilled in the art; Dill and McCallum., Science 338:1042-1046 (2012). Several algorithms for predicting protein structures and the gene sequences that encode these have been developed, and many of these algorithms can be found at the National Center for Biotechnology Information (on the World Wide Web at ncbi.nlm.nih.gov/guide/proteins/) and at the Bioinformatics Resource Portal (on the World Wide Web at expasy.org/proteomics). Thus, the foregoing examples demonstrate that those of skill in the art can recognize sequence motifs and structural conformations that may be used to define structural and functional domains in accordance with the disclosure.

Framework modifications can be made to antibodies to decrease immunogenicity, for example, by “backmutating” one or more framework residues to a corresponding germline sequence.

It is also contemplated that the antigen-binding domain may be multi-specific or multivalent by multimerizing the antigen-binding domain with VH and VL region pairs that bind either the same antigen (multi-valent) or a different antigen (multi-specific).

In some aspects, also contemplated are glycosylation variants of antibodies, wherein the number and/or type of glycosylation site(s) has been altered compared to the amino acid sequences of the parent polypeptide. Glycosylation of the polypeptides can be altered, for example, by modifying one or more sites of glycosylation within the polypeptide sequence to increase the affinity of the polypeptide for antigen (U.S. Pat. Nos. 5,714,350 and 6,350,861). In certain embodiments, antibody protein variants comprise a greater or a lesser number of N-linked glycosylation sites than the native antibody. An N-linked glycosylation site is characterized by the sequence: Asn-X-Ser or Asn-X-Thr, wherein the amino acid residue designated as X may be any amino acid residue except proline. The substitution of amino acid residues to create this sequence provides a potential new site for the addition of an N-linked carbohydrate chain. Alternatively, substitutions that eliminate or alter this sequence will prevent addition of an N-linked carbohydrate chain present in the native polypeptide. For example, the glycosylation can be reduced by the deletion of an Asn or by substituting the Asn with a different amino acid. In other embodiments, one or more new N-linked glycosylation sites are created. Antibodies typically have an N-linked glycosylation site in the Fc region.

Additional antibody variants include cysteine variants, wherein one or more cysteine residues in the parent or native amino acid sequence are deleted from or substituted with another amino acid (e.g., serine). Cysteine variants are useful, inter alia, when antibodies must be refolded into a biologically active conformation. Cysteine variants may have fewer cysteine residues than the native antibody and typically have an even number to minimize interactions resulting from unpaired cysteines.

In some aspects, the polypeptides can be pegylated to increase biological half-life by reacting the polypeptide with polyethylene glycol (PEG) or a reactive ester or aldehyde derivative of PEG, under conditions in which one or more PEG groups become attached to the polypeptide. Polypeptide pegylation may be carried out by an acylation reaction or an alkylation reaction with a reactive PEG molecule (or an analogous reactive water-soluble polymer). Methods for pegylating proteins are known in the art and can be applied to the polypeptides of the disclosure to obtain PEGylated derivatives of antibodies. See, e.g., EP 0 154 316 and EP 0 401 384. In some aspects, the antibody is conjugated or otherwise linked to transthyretin (TTR) or a TTR variant. The TTR or TTR variant can be chemically modified with, for example, a chemical selected from the group consisting of dextran, poly(n-vinyl pyrrolidone), polyethylene glycols, propropylene glycol homopolymers, polypropylene oxide/ethylene oxide co-polymers, polyoxyethylated polyols, and polyvinyl alcohols. As used herein, the term “polyethylene glycol” is intended to encompass any of the forms of PEG that have been used to derivatize other proteins.

Derivatives of the antibodies and antigen binding fragments that are described herein are also provided. The derivatized antibody or fragment thereof may comprise any molecule or substance that imparts a desired property to the antibody or fragment. The derivatized antibody can comprise, for example, a detectable (or labeling) moiety (e.g., a radioactive, colorimetric, antigenic, or enzymatic molecule, or a detectable bead), a molecule that binds to another molecule (e.g., biotin or streptavidin), a therapeutic or diagnostic moiety (e.g., a radioactive, cytotoxic, or pharmaceutically active moiety), or a molecule that increases the suitability of the antibody for a particular use (e.g., administration to a subject, such as a human subject, or other in vivo or in vitro uses). Optionally, an antibody or an immunological portion of an antibody can be chemically conjugated to, or expressed as, a fusion protein with other proteins. In some aspects, polypeptides may be chemically modified by conjugating or fusing the polypeptide to serum protein, such as human serum albumin, to increase half-life of the resulting molecule. See, e.g., EP 0322094 and EP 0 486 525. In some aspects, the polypeptides may also be conjugated to a therapeutic agent to provide a therapy in combination with the therapeutic effect of the polypeptide.

In some aspects, contemplated are immunoconjugates comprising an antibody or antigen-binding fragment thereof conjugated to a cytotoxic agent such as a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate).

B. Example Antibodies

Example antibodies that are capable of binding to and activating PLXDC1 are disclosed in the experimental examples (e.g., Table 6). In one embodiment, an antibody or antigen binding fragment thereof is provided, which has specificity to the human plexin domain-containing 1 (PLXDC1) protein. In some embodiments, the antibody or antigen binding fragment thereof comprises a heavy chain variable region (VH) and a light chain variable region (VL), the VH comprises a VH complementarity-determining region (CDR) CDR1, a VH CDR2, a VH CDR3, the VL comprises a VL CDR1, a VL CDR2, and a VL CDR3. In some embodiments, the VH CDR1, VH CDR2, VH CDR3, VL CDR1, VL CDR2, and VL CDR3 are those of an antibody selected from Table 6.

In one embodiment, the VH CDR1, VH CDR2, VH CDR3, VL CDR1, VL CDR2, and VL CDR3 are those of antibody 1-A1. In one embodiment, the VH CDR1, VH CDR2, VH CDR3, VL CDR1, VL CDR2, and VL CDR3 are those of antibody 1-A5. In one embodiment, the VH CDR1, VH CDR2, VH CDR3, VL CDR1, VL CDR2, and VL CDR3 are those of antibody 1-H10. In one embodiment, the VH CDR1, VH CDR2, VH CDR3, VL CDR1, VL CDR2, and VL CDR3 are those of antibody 2-B4. In one embodiment, the VH CDR1, VH CDR2, VH CDR3, VL CDR1, VL CDR2, and VL CDR3 are those of antibody 2-B5. In one embodiment, the VH CDR1, VH CDR2, VH CDR3, VL CDR1, VL CDR2, and VL CDR3 are those of antibody 2-F7. In one embodiment, the VH CDR1, VH CDR2, VH CDR3, VL CDR1, VL CDR2, and VL CDR3 are those of antibody 2-F8.

In one embodiment, the VH CDR1, VH CDR2, VH CDR3, VL CDR1, VL CDR2, and VL CDR3 are those of antibody 2-G4. In one embodiment, the VH CDR1, VH CDR2, VH CDR3, VL CDR1, VL CDR2, and VL CDR3 are those of antibody 2-H2. In one embodiment, the VH CDR1, VH CDR2, VH CDR3, VL CDR1, VL CDR2, and VL CDR3 are those of antibody 2-H9. In one embodiment, the VH CDR1, VH CDR2, VH CDR3, VL CDR1, VL CDR2, and VL CDR3 are those of antibody 3-A7. In one embodiment, the VH CDR1, VH CDR2, VH CDR3, VL CDR1, VL CDR2, and VL CDR3 are those of antibody 3-A9. In one embodiment, the VH CDR1, VH CDR2, VH CDR3, VL CDR1, VL CDR2, and VL CDR3 are those of antibody 3-B3. In one embodiment, the VH CDR1, VH CDR2, VH CDR3, VL CDR1, VL CDR2, and VL CDR3 are those of antibody 3-C₄.

In one embodiment, the VH CDR1, VH CDR2, VH CDR3, VL CDR1, VL CDR2, and VL CDR3 are those of antibody 3-C₆. In one embodiment, the VH CDR1, VH CDR2, VH CDR3, VL CDR1, VL CDR2, and VL CDR3 are those of antibody 3-C₈. In one embodiment, the VH CDR1, VH CDR2, VH CDR3, VL CDR1, VL CDR2, and VL CDR3 are those of antibody 3-C₉. In one embodiment, the VH CDR1, VH CDR2, VH CDR3, VL CDR1, VL CDR2, and VL CDR3 are those of antibody 3-C₁₁. In one embodiment, the VH CDR1, VH CDR2, VH CDR3, VL CDR1, VL CDR2, and VL CDR3 are those of antibody 3-C₁₂. In one embodiment, the VH CDR1, VH CDR2, VH CDR3, VL CDR1, VL CDR2, and VL CDR3 are those of antibody 3-D3. In one embodiment, the VH CDR1, VH CDR2, VH CDR3, VL CDR1, VL CDR2, and VL CDR3 are those of antibody 3-D6.

In one embodiment, the VH CDR1, VH CDR2, VH CDR3, VL CDR1, VL CDR2, and VL CDR3 are those of antibody 3-D7. In one embodiment, the VH CDR1, VH CDR2, VH CDR3, VL CDR1, VL CDR2, and VL CDR3 are those of antibody 3-D12. In one embodiment, the VH CDR1, VH CDR2, VH CDR3, VL CDR1, VL CDR2, and VL CDR3 are those of antibody 3-E2. In one embodiment, the VH CDR1, VH CDR2, VH CDR3, VL CDR1, VL CDR2, and VL CDR3 are those of antibody 3-E5. In one embodiment, the VH CDR1, VH CDR2, VH CDR3, VL CDR1, VL CDR2, and VL CDR3 are those of antibody 3-E7. In one embodiment, the VH CDR1, VH CDR2, VH CDR3, VL CDR1, VL CDR2, and VL CDR3 are those of antibody 3-E8. In one embodiment, the VH CDR1, VH CDR2, VH CDR3, VL CDR1, VL CDR2, and VL CDR3 are those of antibody 3-E9.

In one embodiment, the VH CDR1, VH CDR2, VH CDR3, VL CDR1, VL CDR2, and VL CDR3 are those of antibody 3-F5. In one embodiment, the VH CDR1, VH CDR2, VH CDR3, VL CDR1, VL CDR2, and VL CDR3 are those of antibody 3-F6. In one embodiment, the VH CDR1, VH CDR2, VH CDR3, VL CDR1, VL CDR2, and VL CDR3 are those of antibody 3-F12. In one embodiment, the VH CDR1, VH CDR2, VH CDR3, VL CDR1, VL CDR2, and VL CDR3 are those of antibody 3-G4. In one embodiment, the VH CDR1, VH CDR2, VH CDR3, VL CDR1, VL CDR2, and VL CDR3 are those of antibody 3-G5. In one embodiment, the VH CDR1, VH CDR2, VH CDR3, VL CDR1, VL CDR2, and VL CDR3 are those of antibody 3-G6. In one embodiment, the VH CDR1, VH CDR2, VH CDR3, VL CDR1, VL CDR2, and VL CDR3 are those of antibody 3-G7.

In one embodiment, the VH CDR1, VH CDR2, VH CDR3, VL CDR1, VL CDR2, and VL CDR3 are those of antibody 3-G8. In one embodiment, the VH CDR1, VH CDR2, VH CDR3, VL CDR1, VL CDR2, and VL CDR3 are those of antibody 3-H1. In one embodiment, the VH CDR1, VH CDR2, VH CDR3, VL CDR1, VL CDR2, and VL CDR3 are those of antibody 3-H4. In one embodiment, the VH CDR1, VH CDR2, VH CDR3, VL CDR1, VL CDR2, and VL CDR3 are those of antibody 4-A7. In one embodiment, the VH CDR1, VH CDR2, VH CDR3, VL CDR1, VL CDR2, and VL CDR3 are those of antibody 4-A8. In one embodiment, the VH CDR1, VH CDR2, VH CDR3, VL CDR1, VL CDR2, and VL CDR3 are those of antibody 4-B1. In one embodiment, the VH CDR1, VH CDR2, VH CDR3, VL CDR1, VL CDR2, and VL CDR3 are those of antibody 4-B2.

In one embodiment, the VH CDR1, VH CDR2, VH CDR3, VL CDR1, VL CDR2, and VL CDR3 are those of antibody 4-B11. In one embodiment, the VH CDR1, VH CDR2, VH CDR3, VL CDR1, VL CDR2, and VL CDR3 are those of antibody 4-D12. In one embodiment, the VH CDR1, VH CDR2, VH CDR3, VL CDR1, VL CDR2, and VL CDR3 are those of antibody 4-F4. In one embodiment, the VH CDR1, VH CDR2, VH CDR3, VL CDR1, VL CDR2, and VL CDR3 are those of antibody 4-G12. In one embodiment, the VH CDR1, VH CDR2, VH CDR3, VL CDR1, VL CDR2, and VL CDR3 are those of antibody 5-C₉. In one embodiment, the VH CDR1, VH CDR2, VH CDR3, VL CDR1, VL CDR2, and VL CDR3 are those of antibody 5-E2. In one embodiment, the VH CDR1, VH CDR2, VH CDR3, VL CDR1, VL CDR2, and VL CDR3 are those of antibody 5-E12.

In one embodiment, the VH CDR1, VH CDR2, VH CDR3, VL CDR1, VL CDR2, and VL CDR3 are those of antibody 6-G4. In one embodiment, the VH CDR1, VH CDR2, VH CDR3, VL CDR1, VL CDR2, and VL CDR3 are those of antibody 6-H5. In one embodiment, the VH CDR1, VH CDR2, VH CDR3, VL CDR1, VL CDR2, and VL CDR3 are those of antibody 8-A2. In one embodiment, the VH CDR1, VH CDR2, VH CDR3, VL CDR1, VL CDR2, and VL CDR3 are those of antibody 8-A4. In one embodiment, the VH CDR1, VH CDR2, VH CDR3, VL CDR1, VL CDR2, and VL CDR3 are those of antibody 8-A6. In one embodiment, the VH CDR1, VH CDR2, VH CDR3, VL CDR1, VL CDR2, and VL CDR3 are those of antibody 8-A7. In one embodiment, the VH CDR1, VH CDR2, VH CDR3, VL CDR1, VL CDR2, and VL CDR3 are those of antibody 8-A8.

In one embodiment, the VH CDR1, VH CDR2, VH CDR3, VL CDR1, VL CDR2, and VL CDR3 are those of antibody 8-A9. In one embodiment, the VH CDR1, VH CDR2, VH CDR3, VL CDR1, VL CDR2, and VL CDR3 are those of antibody 8-C₄. In one embodiment, the VH CDR1, VH CDR2, VH CDR3, VL CDR1, VL CDR2, and VL CDR3 are those of antibody 8-C₆. In one embodiment, the VH CDR1, VH CDR2, VH CDR3, VL CDR1, VL CDR2, and VL CDR3 are those of antibody 8-C₉. In one embodiment, the VH CDR1, VH CDR2, VH CDR3, VL CDR1, VL CDR2, and VL CDR3 are those of antibody 8-C₁₀. In one embodiment, the VH CDR1, VH CDR2, VH CDR3, VL CDR1, VL CDR2, and VL CDR3 are those of antibody 8-C₁₁. In one embodiment, the VH CDR1, VH CDR2, VH CDR3, VL CDR1, VL CDR2, and VL CDR3 are those of antibody 8-D11.

In one embodiment, the VH CDR1, VH CDR2, VH CDR3, VL CDR1, VL CDR2, and VL CDR3 are those of antibody 8-D4. In one embodiment, the VH CDR1, VH CDR2, VH CDR3, VL CDR1, VL CDR2, and VL CDR3 are those of antibody 8-D7. In one embodiment, the VH CDR1, VH CDR2, VH CDR3, VL CDR1, VL CDR2, and VL CDR3 are those of antibody 8-E2. In one embodiment, the VH CDR1, VH CDR2, VH CDR3, VL CDR1, VL CDR2, and VL CDR3 are those of antibody 8-E3. In one embodiment, the VH CDR1, VH CDR2, VH CDR3, VL CDR1, VL CDR2, and VL CDR3 are those of antibody 8-E9. In one embodiment, the VH CDR1, VH CDR2, VH CDR3, VL CDR1, VL CDR2, and VL CDR3 are those of antibody 8-F1. In one embodiment, the VH CDR1, VH CDR2, VH CDR3, VL CDR1, VL CDR2, and VL CDR3 are those of antibody 8-F2.

In one embodiment, the VH CDR1, VH CDR2, VH CDR3, VL CDR1, VL CDR2, and VL CDR3 are those of antibody 8-F4. In one embodiment, the VH CDR1, VH CDR2, VH CDR3, VL CDR1, VL CDR2, and VL CDR3 are those of antibody 8-F6. In one embodiment, the VH CDR1, VH CDR2, VH CDR3, VL CDR1, VL CDR2, and VL CDR3 are those of antibody 8-F11. In one embodiment, the VH CDR1, VH CDR2, VH CDR3, VL CDR1, VL CDR2, and VL CDR3 are those of antibody 8-G12. In one embodiment, the VH CDR1, VH CDR2, VH CDR3, VL CDR1, VL CDR2, and VL CDR3 are those of antibody 8-H7. In one embodiment, the VH CDR1, VH CDR2, VH CDR3, VL CDR1, VL CDR2, and VL CDR3 are those of antibody 9-A6. In one embodiment, the VH CDR1, VH CDR2, VH CDR3, VL CDR1, VL CDR2, and VL CDR3 are those of antibody 9-B3.

In one embodiment, the VH CDR1, VH CDR2, VH CDR3, VL CDR1, VL CDR2, and VL CDR3 are those of antibody 9-B10. In one embodiment, the VH CDR1, VH CDR2, VH CDR3, VL CDR1, VL CDR2, and VL CDR3 are those of antibody 9-C₂. In one embodiment, the VH CDR1, VH CDR2, VH CDR3, VL CDR1, VL CDR2, and VL CDR3 are those of antibody 9-D8. In one embodiment, the VH CDR1, VH CDR2, VH CDR3, VL CDR1, VL CDR2, and VL CDR3 are those of antibody 9-E4. In one embodiment, the VH CDR1, VH CDR2, VH CDR3, VL CDR1, VL CDR2, and VL CDR3 are those of antibody 9-E5. In one embodiment, the VH CDR1, VH CDR2, VH CDR3, VL CDR1, VL CDR2, and VL CDR3 are those of antibody 9-G1. In one embodiment, the VH CDR1, VH CDR2, VH CDR3, VL CDR1, VL CDR2, and VL CDR3 are those of antibody 9-G3.

In one embodiment, the VH CDR1, VH CDR2, VH CDR3, VL CDR1, VL CDR2, and VL CDR3 are those of antibody 9-H9. In one embodiment, the VH CDR1, VH CDR2, VH CDR3, VL CDR1, VL CDR2, and VL CDR3 are those of antibody 2-C₈. In one embodiment, the VH CDR1, VH CDR2, VH CDR3, VL CDR1, VL CDR2, and VL CDR3 are those of antibody 8-D9. In one embodiment, the VH CDR1, VH CDR2, VH CDR3, VL CDR1, VL CDR2, and VL CDR3 are those of antibody 10-A9. In one embodiment, the VH CDR1, VH CDR2, VH CDR3, VL CDR1, VL CDR2, and VL CDR3 are those of antibody 10-B2. In one embodiment, the VH CDR1, VH CDR2, VH CDR3, VL CDR1, VL CDR2, and VL CDR3 are those of antibody 10-B5. In one embodiment, the VH CDR1, VH CDR2, VH CDR3, VL CDR1, VL CDR2, and VL CDR3 are those of antibody 10-C₁.

In one embodiment, the VH CDR1, VH CDR2, VH CDR3, VL CDR1, VL CDR2, and VL CDR3 are those of antibody 10-C₃. In one embodiment, the VH CDR1, VH CDR2, VH CDR3, VL CDR1, VL CDR2, and VL CDR3 are those of antibody 10-C₉. In one embodiment, the VH CDR1, VH CDR2, VH CDR3, VL CDR1, VL CDR2, and VL CDR3 are those of antibody 10-C₁₁. In one embodiment, the VH CDR1, VH CDR2, VH CDR3, VL CDR1, VL CDR2, and VL CDR3 are those of antibody 10-D7. In one embodiment, the VH CDR1, VH CDR2, VH CDR3, VL CDR1, VL CDR2, and VL CDR3 are those of antibody 10-E1. In one embodiment, the VH CDR1, VH CDR2, VH CDR3, VL CDR1, VL CDR2, and VL CDR3 are those of antibody 10-E6. In one embodiment, the VH CDR1, VH CDR2, VH CDR3, VL CDR1, VL CDR2, and VL CDR3 are those of antibody 10-E7.

In one embodiment, the VH CDR1, VH CDR2, VH CDR3, VL CDR1, VL CDR2, and VL CDR3 are those of antibody 10-F11. In one embodiment, the VH CDR1, VH CDR2, VH CDR3, VL CDR1, VL CDR2, and VL CDR3 are those of antibody 10-F12. In one embodiment, the VH CDR1, VH CDR2, VH CDR3, VL CDR1, VL CDR2, and VL CDR3 are those of antibody 10-G5. In one embodiment, the VH CDR1, VH CDR2, VH CDR3, VL CDR1, VL CDR2, and VL CDR3 are those of antibody 11-B6. In one embodiment, the VH CDR1, VH CDR2, VH CDR3, VL CDR1, VL CDR2, and VL CDR3 are those of antibody 11-D8. In one embodiment, the VH CDR1, VH CDR2, VH CDR3, VL CDR1, VL CDR2, and VL CDR3 are those of antibody 11-D10. In one embodiment, the VH CDR1, VH CDR2, VH CDR3, VL CDR1, VL CDR2, and VL CDR3 are those of antibody 11-D11.

In one embodiment, the VH CDR1, VH CDR2, VH CDR3, VL CDR1, VL CDR2, and VL CDR3 are those of antibody 11-G2. In one embodiment, the VH CDR1, VH CDR2, VH CDR3, VL CDR1, VL CDR2, and VL CDR3 are those of antibody 11-G5. In one embodiment, the VH CDR1, VH CDR2, VH CDR3, VL CDR1, VL CDR2, and VL CDR3 are those of antibody 11-G9. In one embodiment, the VH CDR1, VH CDR2, VH CDR3, VL CDR1, VL CDR2, and VL CDR3 are those of antibody 11-H8. In one embodiment, the VH CDR1, VH CDR2, VH CDR3, VL CDR1, VL CDR2, and VL CDR3 are those of antibody 11-H11.

Example CDR sequences (Kabat) from these antibodies are shown in Table 7. In one embodiment, the VH CDR1, VH CDR2, VH CDR3, VL CDR1, VL CDR2, and VL CDR3 comprise the amino acid sequences of SEQ ID NO:224, 248, 341, 435, 497, and 517, respectively. In one embodiment, the VH CDR1, VH CDR2, VH CDR3, VL CDR1, VL CDR2, and VL CDR3 comprise the amino acid sequences of SEQ ID NO:225, 249, 342, 436, 498, and 518, respectively. In one embodiment, the VH CDR1, VH CDR2, VH CDR3, VL CDR1, VL CDR2, and VL CDR3 comprise the amino acid sequences of SEQ ID NO:226, 250, 343, 437, 499, and 519, respectively. In one embodiment, the VH CDR1, VH CDR2, VH CDR3, VL CDR1, VL CDR2, and VL CDR3 comprise the amino acid sequences of SEQ ID NO:227, 251, 344, 438, 500, and 520, respectively. In one embodiment, the VH CDR1, VH CDR2, VH CDR3, VL CDR1, VL CDR2, and VL CDR3 comprise the amino acid sequences of SEQ ID NO:228, 252, 345, 439, 501, and 521, respectively.

In one embodiment, the VH CDR1, VH CDR2, VH CDR3, VL CDR1, VL CDR2, and VL CDR3 comprise the amino acid sequences of SEQ ID NO:229, 253, 346, 440, 501, and 522, respectively. In one embodiment, the VH CDR1, VH CDR2, VH CDR3, VL CDR1, VL CDR2, and VL CDR3 comprise the amino acid sequences of SEQ ID NO:226, 254, 347, 441, 500, and 523, respectively. In one embodiment, the VH CDR1, VH CDR2, VH CDR3, VL CDR1, VL CDR2, and VL CDR3 comprise the amino acid sequences of SEQ ID NO:230, 255, 348, 442, 502, and 524, respectively. In one embodiment, the VH CDR1, VH CDR2, VH CDR3, VL CDR1, VL CDR2, and VL CDR3 comprise the amino acid sequences of SEQ ID NO:231, 256, 349, 443, 500, and 525, respectively. In one embodiment, the VH CDR1, VH CDR2, VH CDR3, VL CDR1, VL CDR2, and VL CDR3 comprise the amino acid sequences of SEQ ID NO:232, 257, 350, 444, 500, and 526, respectively. In one embodiment, the VH CDR1, VH CDR2, VH CDR3, VL CDR1, VL CDR2, and VL CDR3 comprise the amino acid sequences of SEQ ID NO:227, 258, 351, 445, 500, and 527, respectively.

In one embodiment, the VH CDR1, VH CDR2, VH CDR3, VL CDR1, VL CDR2, and VL CDR3 comprise the amino acid sequences of SEQ ID NO:226, 259, 352, 446, 502, and 528, respectively. In one embodiment, the VH CDR1, VH CDR2, VH CDR3, VL CDR1, VL CDR2, and VL CDR3 comprise the amino acid sequences of SEQ ID NO:224, 260, 353, 445, 500, and 523, respectively. In one embodiment, the VH CDR1, VH CDR2, VH CDR3, VL CDR1, VL CDR2, and VL CDR3 comprise the amino acid sequences of SEQ ID NO:224, 261, 354, 447, 500, and 529, respectively. In one embodiment, the VH CDR1, VH CDR2, VH CDR3, VL CDR1, VL CDR2, and VL CDR3 comprise the amino acid sequences of SEQ ID NO:232, 262, 355, 448, 503, and 530, respectively. In one embodiment, the VH CDR1, VH CDR2, VH CDR3, VL CDR1, VL CDR2, and VL CDR3 comprise the amino acid sequences of SEQ ID NO:229, 263, 356, 449, 500, and 531, respectively. In one embodiment, the VH CDR1, VH CDR2, VH CDR3, VL CDR1, VL CDR2, and VL CDR3 comprise the amino acid sequences of SEQ ID NO:226, 264, 357, 450, 500, and 532, respectively.

In one embodiment, the VH CDR1, VH CDR2, VH CDR3, VL CDR1, VL CDR2, and VL CDR3 comprise the amino acid sequences of SEQ ID NO:224, 265, 358, 451, 504, and 523, respectively. In one embodiment, the VH CDR1, VH CDR2, VH CDR3, VL CDR1, VL CDR2, and VL CDR3 comprise the amino acid sequences of SEQ ID NO:229, 266, 359, 448, 500, and 533, respectively. In one embodiment, the VH CDR1, VH CDR2, VH CDR3, VL CDR1, VL CDR2, and VL CDR3 comprise the amino acid sequences of SEQ ID NO:226, 267, 360, 452, 502, and 534, respectively. In one embodiment, the VH CDR1, VH CDR2, VH CDR3, VL CDR1, VL CDR2, and VL CDR3 comprise the amino acid sequences of SEQ ID NO:233, 268, 361, 439, 501, and 535, respectively. In one embodiment, the VH CDR1, VH CDR2, VH CDR3, VL CDR1, VL CDR2, and VL CDR3 comprise the amino acid sequences of SEQ ID NO:226, 269, 362, 453, 502, and 536, respectively.

In one embodiment, the VH CDR1, VH CDR2, VH CDR3, VL CDR1, VL CDR2, and VL CDR3 comprise the amino acid sequences of SEQ ID NO:234, 270, 363, 454, 502, and 537, respectively. In one embodiment, the VH CDR1, VH CDR2, VH CDR3, VL CDR1, VL CDR2, and VL CDR3 comprise the amino acid sequences of SEQ ID NO:226, 271, 364, 444, 500, and 538, respectively. In one embodiment, the VH CDR1, VH CDR2, VH CDR3, VL CDR1, VL CDR2, and VL CDR3 comprise the amino acid sequences of SEQ ID NO:226, 272, 365, 455, 502, and 539, respectively. In one embodiment, the VH CDR1, VH CDR2, VH CDR3, VL CDR1, VL CDR2, and VL CDR3 comprise the amino acid sequences of SEQ ID NO:226, 273, 366, 456, 501, and 540, respectively. In one embodiment, the VH CDR1, VH CDR2, VH CDR3, VL CDR1, VL CDR2, and VL CDR3 comprise the amino acid sequences of SEQ ID NO:229, 274, 367, 457, 501, and 541, respectively.

In one embodiment, the VH CDR1, VH CDR2, VH CDR3, VL CDR1, VL CDR2, and VL CDR3 comprise the amino acid sequences of SEQ ID NO:224, 275, 368, 444, 503, and 542, respectively. In one embodiment, the VH CDR1, VH CDR2, VH CDR3, VL CDR1, VL CDR2, and VL CDR3 comprise the amino acid sequences of SEQ ID NO:234, 270, 363, 446, 502, and 543, respectively. In one embodiment, the VH CDR1, VH CDR2, VH CDR3, VL CDR1, VL CDR2, and VL CDR3 comprise the amino acid sequences of SEQ ID NO:229, 276, 369, 458, 502, and 544, respectively. In one embodiment, the VH CDR1, VH CDR2, VH CDR3, VL CDR1, VL CDR2, and VL CDR3 comprise the amino acid sequences of SEQ ID NO:229, 277, 370, 454, 502, and 545, respectively. In one embodiment, the VH CDR1, VH CDR2, VH CDR3, VL CDR1, VL CDR2, and VL CDR3 comprise the amino acid sequences of SEQ ID NO:232, 278, 371, 459, 500, and 531, respectively.

In one embodiment, the VH CDR1, VH CDR2, VH CDR3, VL CDR1, VL CDR2, and VL CDR3 comprise the amino acid sequences of SEQ ID NO:224, 279, 372, 460, 504, and 531, respectively. In one embodiment, the VH CDR1, VH CDR2, VH CDR3, VL CDR1, VL CDR2, and VL CDR3 comprise the amino acid sequences of SEQ ID NO:231, 280, 373, 444, 505, and 546, respectively. In one embodiment, the VH CDR1, VH CDR2, VH CDR3, VL CDR1, VL CDR2, and VL CDR3 comprise the amino acid sequences of SEQ ID NO:235, 281, 374, 461, 501, and 547, respectively. In one embodiment, the VH CDR1, VH CDR2, VH CDR3, VL CDR1, VL CDR2, and VL CDR3 comprise the amino acid sequences of SEQ ID NO:226, 282, 375, 462, 500, and 523, respectively. In one embodiment, the VH CDR1, VH CDR2, VH CDR3, VL CDR1, VL CDR2, and VL CDR3 comprise the amino acid sequences of SEQ ID NO:226, 283, 376, 463, 506, and 548, respectively.

In one embodiment, the VH CDR1, VH CDR2, VH CDR3, VL CDR1, VL CDR2, and VL CDR3 comprise the amino acid sequences of SEQ ID NO:226, 284, 377, 464, 501, and 549, respectively. In one embodiment, the VH CDR1, VH CDR2, VH CDR3, VL CDR1, VL CDR2, and VL CDR3 comprise the amino acid sequences of SEQ ID NO:236, 285, 378, 435, 507, and 550, respectively. In one embodiment, the VH CDR1, VH CDR2, VH CDR3, VL CDR1, VL CDR2, and VL CDR3 comprise the amino acid sequences of SEQ ID NO:226, 286, 379, 465, 508, and 551, respectively. In one embodiment, the VH CDR1, VH CDR2, VH CDR3, VL CDR1, VL CDR2, and VL CDR3 comprise the amino acid sequences of SEQ ID NO:229, 287, 380, 435, 509, and 552, respectively. In one embodiment, the VH CDR1, VH CDR2, VH CDR3, VL CDR1, VL CDR2, and VL CDR3 comprise the amino acid sequences of SEQ ID NO:237, 288, 381, 466, 509, and 553, respectively.

In one embodiment, the VH CDR1, VH CDR2, VH CDR3, VL CDR1, VL CDR2, and VL CDR3 comprise the amino acid sequences of SEQ ID NO:238, 289, 382, 467, 510, and 554, respectively. In one embodiment, the VH CDR1, VH CDR2, VH CDR3, VL CDR1, VL CDR2, and VL CDR3 comprise the amino acid sequences of SEQ ID NO:226, 290, 376, 435, 511, and 555, respectively. In one embodiment, the VH CDR1, VH CDR2, VH CDR3, VL CDR1, VL CDR2, and VL CDR3 comprise the amino acid sequences of SEQ ID NO:239, 291, 383, 435, 508, and 556, respectively. In one embodiment, the VH CDR1, VH CDR2, VH CDR3, VL CDR1, VL CDR2, and VL CDR3 comprise the amino acid sequences of SEQ ID NO:224, 292, 384, 468, 512, and 557, respectively. In one embodiment, the VH CDR1, VH CDR2, VH CDR3, VL CDR1, VL CDR2, and VL CDR3 comprise the amino acid sequences of SEQ ID NO:240, 293, 385, 469, 502, and 558, respectively.

In one embodiment, the VH CDR1, VH CDR2, VH CDR3, VL CDR1, VL CDR2, and VL CDR3 comprise the amino acid sequences of SEQ ID NO:231, 294, 386, 445, 500, and 559, respectively. In one embodiment, the VH CDR1, VH CDR2, VH CDR3, VL CDR1, VL CDR2, and VL CDR3 comprise the amino acid sequences of SEQ ID NO:226, 295, 387, 470, 501, and 560, respectively. In one embodiment, the VH CDR1, VH CDR2, VH CDR3, VL CDR1, VL CDR2, and VL CDR3 comprise the amino acid sequences of SEQ ID NO:229, 296, 388, 435, 498, and 561, respectively. In one embodiment, the VH CDR1, VH CDR2, VH CDR3, VL CDR1, VL CDR2, and VL CDR3 comprise the amino acid sequences of SEQ ID NO:226, 297, 389, 435, 498, and 562, respectively. In one embodiment, the VH CDR1, VH CDR2, VH CDR3, VL CDR1, VL CDR2, and VL CDR3 comprise the amino acid sequences of SEQ ID NO:241, 298, 390, 454, 513, and 563, respectively.

In one embodiment, the VH CDR1, VH CDR2, VH CDR3, VL CDR1, VL CDR2, and VL CDR3 comprise the amino acid sequences of SEQ ID NO:224, 299, 376, 459, 505, and 564, respectively. In one embodiment, the VH CDR1, VH CDR2, VH CDR3, VL CDR1, VL CDR2, and VL CDR3 comprise the amino acid sequences of SEQ ID NO:229, 300, 391, 445, 500, and 565, respectively. In one embodiment, the VH CDR1, VH CDR2, VH CDR3, VL CDR1, VL CDR2, and VL CDR3 comprise the amino acid sequences of SEQ ID NO:226, 301, 392, 471, 501, and 566, respectively. In one embodiment, the VH CDR1, VH CDR2, VH CDR3, VL CDR1, VL CDR2, and VL CDR3 comprise the amino acid sequences of SEQ ID NO:226, 302, 393, 472, 500, and 567, respectively. In one embodiment, the VH CDR1, VH CDR2, VH CDR3, VL CDR1, VL CDR2, and VL CDR3 comprise the amino acid sequences of SEQ ID NO:226, 303, 394, 473, 500, and 523, respectively.

In one embodiment, the VH CDR1, VH CDR2, VH CDR3, VL CDR1, VL CDR2, and VL CDR3 comprise the amino acid sequences of SEQ ID NO:226, 304, 395, 462, 500, and 523, respectively. In one embodiment, the VH CDR1, VH CDR2, VH CDR3, VL CDR1, VL CDR2, and VL CDR3 comprise the amino acid sequences of SEQ ID NO:229, 305, 396, 444, 500, and 568, respectively. In one embodiment, the VH CDR1, VH CDR2, VH CDR3, VL CDR1, VL CDR2, and VL CDR3 comprise the amino acid sequences of SEQ ID NO:224, 306, 397, 474, 505, and 569, respectively. In one embodiment, the VH CDR1, VH CDR2, VH CDR3, VL CDR1, VL CDR2, and VL CDR3 comprise the amino acid sequences of SEQ ID NO:224, 307, 398, 463, 500, and 570, respectively. In one embodiment, the VH CDR1, VH CDR2, VH CDR3, VL CDR1, VL CDR2, and VL CDR3 comprise the amino acid sequences of SEQ ID NO:224, 308, 399, 472, 503, and 531, respectively.

In one embodiment, the VH CDR1, VH CDR2, VH CDR3, VL CDR1, VL CDR2, and VL CDR3 comprise the amino acid sequences of SEQ ID NO:242, 309, 400, 442, 514, and 571, respectively. In one embodiment, the VH CDR1, VH CDR2, VH CDR3, VL CDR1, VL CDR2, and VL CDR3 comprise the amino acid sequences of SEQ ID NO:224, 310, 376, 475, 500, and 530, respectively. In one embodiment, the VH CDR1, VH CDR2, VH CDR3, VL CDR1, VL CDR2, and VL CDR3 comprise the amino acid sequences of SEQ ID NO:224, 311, 401, 476, 513, and 572, respectively. In one embodiment, the VH CDR1, VH CDR2, VH CDR3, VL CDR1, VL CDR2, and VL CDR3 comprise the amino acid sequences of SEQ ID NO:243, 312, 402, 477, 515, and 573, respectively. In one embodiment, the VH CDR1, VH CDR2, VH CDR3, VL CDR1, VL CDR2, and VL CDR3 comprise the amino acid sequences of SEQ ID NO:226, 313, 403, 478, 516, and 535, respectively.

In one embodiment, the VH CDR1, VH CDR2, VH CDR3, VL CDR1, VL CDR2, and VL CDR3 comprise the amino acid sequences of SEQ ID NO:229, 314, 404, 479, 513, and 574, respectively. In one embodiment, the VH CDR1, VH CDR2, VH CDR3, VL CDR1, VL CDR2, and VL CDR3 comprise the amino acid sequences of SEQ ID NO:229, 315, 405, 439, 516, and 575, respectively. In one embodiment, the VH CDR1, VH CDR2, VH CDR3, VL CDR1, VL CDR2, and VL CDR3 comprise the amino acid sequences of SEQ ID NO:226, 303, 394, 473, 500, and 523, respectively. In one embodiment, the VH CDR1, VH CDR2, VH CDR3, VL CDR1, VL CDR2, and VL CDR3 comprise the amino acid sequences of SEQ ID NO:234, 316, 406, 438, 500, and 523, respectively. In one embodiment, the VH CDR1, VH CDR2, VH CDR3, VL CDR1, VL CDR2, and VL CDR3 comprise the amino acid sequences of SEQ ID NO:224, 317, 407, 439, 516, and 576, respectively.

In one embodiment, the VH CDR1, VH CDR2, VH CDR3, VL CDR1, VL CDR2, and VL CDR3 comprise the amino acid sequences of SEQ ID NO:244, 318, 408, 458, 514, and 577, respectively. In one embodiment, the VH CDR1, VH CDR2, VH CDR3, VL CDR1, VL CDR2, and VL CDR3 comprise the amino acid sequences of SEQ ID NO:234, 270, 363, 446, 502, and 543, respectively. In one embodiment, the VH CDR1, VH CDR2, VH CDR3, VL CDR1, VL CDR2, and VL CDR3 comprise the amino acid sequences of SEQ ID NO:226, 304, 395, 462, 500, and 523, respectively. In one embodiment, the VH CDR1, VH CDR2, VH CDR3, VL CDR1, VL CDR2, and VL CDR3 comprise the amino acid sequences of SEQ ID NO:229, 300, 391, 445, 500, and 565, respectively. In one embodiment, the VH CDR1, VH CDR2, VH CDR3, VL CDR1, VL CDR2, and VL CDR3 comprise the amino acid sequences of SEQ ID NO:231, 280, 373, 438, 500, and 578, respectively.

In one embodiment, the VH CDR1, VH CDR2, VH CDR3, VL CDR1, VL CDR2, and VL CDR3 comprise the amino acid sequences of SEQ ID NO:224, 319, 409, 439, 516, and 579, respectively. In one embodiment, the VH CDR1, VH CDR2, VH CDR3, VL CDR1, VL CDR2, and VL CDR3 comprise the amino acid sequences of SEQ ID NO:224, 317, 407, 439, 516, and 576, respectively. In one embodiment, the VH CDR1, VH CDR2, VH CDR3, VL CDR1, VL CDR2, and VL CDR3 comprise the amino acid sequences of SEQ ID NO:245, 320, 410, 480, 516, and 580, respectively. In one embodiment, the VH CDR1, VH CDR2, VH CDR3, VL CDR1, VL CDR2, and VL CDR3 comprise the amino acid sequences of SEQ ID NO:224, 317, 407, 439, 516, and 576, respectively. In one embodiment, the VH CDR1, VH CDR2, VH CDR3, VL CDR1, VL CDR2, and VL CDR3 comprise the amino acid sequences of SEQ ID NO:242, 321, 411, 481, 500, and 581, respectively.

In one embodiment, the VH CDR1, VH CDR2, VH CDR3, VL CDR1, VL CDR2, and VL CDR3 comprise the amino acid sequences of SEQ ID NO:226, 322, 412, 482, 503, and 582, respectively. In one embodiment, the VH CDR1, VH CDR2, VH CDR3, VL CDR1, VL CDR2, and VL CDR3 comprise the amino acid sequences of SEQ ID NO:224, 323, 413, 474, 500, and 583, respectively. In one embodiment, the VH CDR1, VH CDR2, VH CDR3, VL CDR1, VL CDR2, and VL CDR3 comprise the amino acid sequences of SEQ ID NO:226, 324, 414, 445, 505, and 584, respectively. In one embodiment, the VH CDR1, VH CDR2, VH CDR3, VL CDR1, VL CDR2, and VL CDR3 comprise the amino acid sequences of SEQ ID NO:226, 325, 415, 450, 500, and 585, respectively. In one embodiment, the VH CDR1, VH CDR2, VH CDR3, VL CDR1, VL CDR2, and VL CDR3 comprise the amino acid sequences of SEQ ID NO:243, 312, 416, 477, 515, and 573, respectively.

In one embodiment, the VH CDR1, VH CDR2, VH CDR3, VL CDR1, VL CDR2, and VL CDR3 comprise the amino acid sequences of SEQ ID NO:226, 325, 415, 450, 503, and 585, respectively. In one embodiment, the VH CDR1, VH CDR2, VH CDR3, VL CDR1, VL CDR2, and VL CDR3 comprise the amino acid sequences of SEQ ID NO:229, 300, 417, 445, 500, and 565, respectively. In one embodiment, the VH CDR1, VH CDR2, VH CDR3, VL CDR1, VL CDR2, and VL CDR3 comprise the amino acid sequences of SEQ ID NO:224, 326, 418, 483, 500, and 531, respectively. In one embodiment, the VH CDR1, VH CDR2, VH CDR3, VL CDR1, VL CDR2, and VL CDR3 comprise the amino acid sequences of SEQ ID NO:226, 327, 419, 484, 500, and 529, respectively. In one embodiment, the VH CDR1, VH CDR2, VH CDR3, VL CDR1, VL CDR2, and VL CDR3 comprise the amino acid sequences of SEQ ID NO:226, 279, 420, 485, 500, and 531, respectively.

In one embodiment, the VH CDR1, VH CDR2, VH CDR3, VL CDR1, VL CDR2, and VL CDR3 comprise the amino acid sequences of SEQ ID NO:226, 304, 421, 486, 500, and 523, respectively. In one embodiment, the VH CDR1, VH CDR2, VH CDR3, VL CDR1, VL CDR2, and VL CDR3 comprise the amino acid sequences of SEQ ID NO:224, 328, 422, 470, 501, and 586, respectively. In one embodiment, the VH CDR1, VH CDR2, VH CDR3, VL CDR1, VL CDR2, and VL CDR3 comprise the amino acid sequences of SEQ ID NO:226, 329, 376, 484, 500, and 548, respectively. In one embodiment, the VH CDR1, VH CDR2, VH CDR3, VL CDR1, VL CDR2, and VL CDR3 comprise the amino acid sequences of SEQ ID NO:229, 300, 417, 445, 500, and 565, respectively. In one embodiment, the VH CDR1, VH CDR2, VH CDR3, VL CDR1, VL CDR2, and VL CDR3 comprise the amino acid sequences of SEQ ID NO:224, 330, 423, 487, 505, and 587, respectively.

In one embodiment, the VH CDR1, VH CDR2, VH CDR3, VL CDR1, VL CDR2, and VL CDR3 comprise the amino acid sequences of SEQ ID NO:229, 279, 424, 438, 500, and 588, respectively. In one embodiment, the VH CDR1, VH CDR2, VH CDR3, VL CDR1, VL CDR2, and VL CDR3 comprise the amino acid sequences of SEQ ID NO:246, 331, 425, 488, 501, and 589, respectively. In one embodiment, the VH CDR1, VH CDR2, VH CDR3, VL CDR1, VL CDR2, and VL CDR3 comprise the amino acid sequences of SEQ ID NO:226, 332, 426, 489, 516, and 566, respectively. In one embodiment, the VH CDR1, VH CDR2, VH CDR3, VL CDR1, VL CDR2, and VL CDR3 comprise the amino acid sequences of SEQ ID NO:226, 333, 427, 490, 501, and 590, respectively. In one embodiment, the VH CDR1, VH CDR2, VH CDR3, VL CDR1, VL CDR2, and VL CDR3 comprise the amino acid sequences of SEQ ID NO:247, 334, 428, 491, 516, and 535, respectively.

In one embodiment, the VH CDR1, VH CDR2, VH CDR3, VL CDR1, VL CDR2, and VL CDR3 comprise the amino acid sequences of SEQ ID NO:229, 335, 429, 492, 500, and 567, respectively. In one embodiment, the VH CDR1, VH CDR2, VH CDR3, VL CDR1, VL CDR2, and VL CDR3 comprise the amino acid sequences of SEQ ID NO:224, 336, 430, 493, 502, and 591, respectively. In one embodiment, the VH CDR1, VH CDR2, VH CDR3, VL CDR1, VL CDR2, and VL CDR3 comprise the amino acid sequences of SEQ ID NO:232, 337, 431, 494, 500, and 592, respectively. In one embodiment, the VH CDR1, VH CDR2, VH CDR3, VL CDR1, VL CDR2, and VL CDR3 comprise the amino acid sequences of SEQ ID NO:241, 298, 390, 454, 513, and 563, respectively. In one embodiment, the VH CDR1, VH CDR2, VH CDR3, VL CDR1, VL CDR2, and VL CDR3 comprise the amino acid sequences of SEQ ID NO:226, 338, 432, 495, 502, and 593, respectively.

In one embodiment, the VH CDR1, VH CDR2, VH CDR3, VL CDR1, VL CDR2, and VL CDR3 comprise the amino acid sequences of SEQ ID NO:229, 339, 433, 496, 503, and 529, respectively. In one embodiment, the VH CDR1, VH CDR2, VH CDR3, VL CDR1, VL CDR2, and VL CDR3 comprise the amino acid sequences of SEQ ID NO:226, 340, 434, 438, 500, and 548, respectively. In one embodiment, the VH CDR1, VH CDR2, VH CDR3, VL CDR1, VL CDR2, and VL CDR3 comprise the amino acid sequences of SEQ ID NO:226, 269, 362, 454, 502, and 594, respectively.

In some embodiments, the antibody or antigen binding fragment thereof includes a VH and VL of any of the antibody of Table 6. In one embodiment, the VH and the VL have the amino acid sequences of SEQ ID NO:4 and 5, respectively. In one embodiment, the VH and the VL have the amino acid sequences of SEQ ID NO:6 and 7, respectively. In one embodiment, the VH and the VL have the amino acid sequences of SEQ ID NO:8 and 9, respectively. In one embodiment, the VH and the VL have the amino acid sequences of SEQ ID NO:10 and 11, respectively. In one embodiment, the VH and the VL have the amino acid sequences of SEQ ID NO:12 and 13, respectively. In one embodiment, the VH and the VL have the amino acid sequences of SEQ ID NO:14 and 15, respectively. In one embodiment, the VH and the VL have the amino acid sequences of SEQ ID NO:16 and 17, respectively. In one embodiment, the VH and the VL have the amino acid sequences of SEQ ID NO:18 and 19, respectively.

In one embodiment, the VH and the VL have the amino acid sequences of SEQ ID NO:20 and 21, respectively. In one embodiment, the VH and the VL have the amino acid sequences of SEQ ID NO:22 and 23, respectively. In one embodiment, the VH and the VL have the amino acid sequences of SEQ ID NO:24 and 25, respectively. In one embodiment, the VH and the VL have the amino acid sequences of SEQ ID NO:26 and 27, respectively. In one embodiment, the VH and the VL have the amino acid sequences of SEQ ID NO:28 and 29, respectively. In one embodiment, the VH and the VL have the amino acid sequences of SEQ ID NO:30 and 31, respectively. In one embodiment, the VH and the VL have the amino acid sequences of SEQ ID NO:32 and 33, respectively. In one embodiment, the VH and the VL have the amino acid sequences of SEQ ID NO:34 and 35, respectively. In one embodiment, the VH and the VL have the amino acid sequences of SEQ ID NO:36 and 37, respectively.

In one embodiment, the VH and the VL have the amino acid sequences of SEQ ID NO:38 and 39, respectively. In one embodiment, the VH and the VL have the amino acid sequences of SEQ ID NO:40 and 41, respectively. In one embodiment, the VH and the VL have the amino acid sequences of SEQ ID NO:42 and 43, respectively. In one embodiment, the VH and the VL have the amino acid sequences of SEQ ID NO:44 and 45, respectively. In one embodiment, the VH and the VL have the amino acid sequences of SEQ ID NO:46 and 47, respectively. In one embodiment, the VH and the VL have the amino acid sequences of SEQ ID NO:48 and 49, respectively. In one embodiment, the VH and the VL have the amino acid sequences of SEQ ID NO:50 and 51, respectively. In one embodiment, the VH and the VL have the amino acid sequences of SEQ ID NO:52 and 53, respectively. In one embodiment, the VH and the VL have the amino acid sequences of SEQ ID NO:54 and 55, respectively.

In one embodiment, the VH and the VL have the amino acid sequences of SEQ ID NO:56 and 57, respectively. In one embodiment, the VH and the VL have the amino acid sequences of SEQ ID NO:58 and 59, respectively. In one embodiment, the VH and the VL have the amino acid sequences of SEQ ID NO:60 and 61, respectively. In one embodiment, the VH and the VL have the amino acid sequences of SEQ ID NO:62 and 63, respectively. In one embodiment, the VH and the VL have the amino acid sequences of SEQ ID NO:64 and 65, respectively. In one embodiment, the VH and the VL have the amino acid sequences of SEQ ID NO:66 and 67, respectively. In one embodiment, the VH and the VL have the amino acid sequences of SEQ ID NO:68 and 69, respectively. In one embodiment, the VH and the VL have the amino acid sequences of SEQ ID NO:70 and 71, respectively. In one embodiment, the VH and the VL have the amino acid sequences of SEQ ID NO:72 and 73, respectively.

In one embodiment, the VH and the VL have the amino acid sequences of SEQ ID NO:74 and 75, respectively. In one embodiment, the VH and the VL have the amino acid sequences of SEQ ID NO:76 and 77, respectively. In one embodiment, the VH and the VL have the amino acid sequences of SEQ ID NO:78 and 79, respectively. In one embodiment, the VH and the VL have the amino acid sequences of SEQ ID NO:80 and 81, respectively. In one embodiment, the VH and the VL have the amino acid sequences of SEQ ID NO:82 and 83, respectively. In one embodiment, the VH and the VL have the amino acid sequences of SEQ ID NO:84 and 85, respectively. In one embodiment, the VH and the VL have the amino acid sequences of SEQ ID NO:86 and 87, respectively. In one embodiment, the VH and the VL have the amino acid sequences of SEQ ID NO:88 and 89, respectively. In one embodiment, the VH and the VL have the amino acid sequences of SEQ ID NO:90 and 91, respectively.

In one embodiment, the VH and the VL have the amino acid sequences of SEQ ID NO:92 and 93, respectively. In one embodiment, the VH and the VL have the amino acid sequences of SEQ ID NO:94 and 95, respectively. In one embodiment, the VH and the VL have the amino acid sequences of SEQ ID NO:96 and 97, respectively. In one embodiment, the VH and the VL have the amino acid sequences of SEQ ID NO:98 and 99, respectively. In one embodiment, the VH and the VL have the amino acid sequences of SEQ ID NO:100 and 101, respectively. In one embodiment, the VH and the VL have the amino acid sequences of SEQ ID NO:102 and 103, respectively. In one embodiment, the VH and the VL have the amino acid sequences of SEQ ID NO:104 and 105, respectively. In one embodiment, the VH and the VL have the amino acid sequences of SEQ ID NO:106 and 107, respectively. In one embodiment, the VH and the VL have the amino acid sequences of SEQ ID NO:108 and 109, respectively.

In one embodiment, the VH and the VL have the amino acid sequences of SEQ ID NO:110 and 111, respectively. In one embodiment, the VH and the VL have the amino acid sequences of SEQ ID NO:112 and 113, respectively. In one embodiment, the VH and the VL have the amino acid sequences of SEQ ID NO:114 and 115, respectively. In one embodiment, the VH and the VL have the amino acid sequences of SEQ ID NO:116 and 117, respectively. In one embodiment, the VH and the VL have the amino acid sequences of SEQ ID NO:118 and 119, respectively. In one embodiment, the VH and the VL have the amino acid sequences of SEQ ID NO:120 and 121, respectively. In one embodiment, the VH and the VL have the amino acid sequences of SEQ ID NO:122 and 123, respectively. In one embodiment, the VH and the VL have the amino acid sequences of SEQ ID NO:124 and 125, respectively.

In one embodiment, the VH and the VL have the amino acid sequences of SEQ ID NO:126 and 127, respectively. In one embodiment, the VH and the VL have the amino acid sequences of SEQ ID NO:128 and 129, respectively. In one embodiment, the VH and the VL have the amino acid sequences of SEQ ID NO:130 and 131, respectively. In one embodiment, the VH and the VL have the amino acid sequences of SEQ ID NO:132 and 133, respectively. In one embodiment, the VH and the VL have the amino acid sequences of SEQ ID NO:134 and 135, respectively. In one embodiment, the VH and the VL have the amino acid sequences of SEQ ID NO:136 and 137, respectively. In one embodiment, the VH and the VL have the amino acid sequences of SEQ ID NO:138 and 139, respectively. In one embodiment, the VH and the VL have the amino acid sequences of SEQ ID NO:140 and 141, respectively.

In one embodiment, the VH and the VL have the amino acid sequences of SEQ ID NO:142 and 143, respectively. In one embodiment, the VH and the VL have the amino acid sequences of SEQ ID NO:144 and 145, respectively. In one embodiment, the VH and the VL have the amino acid sequences of SEQ ID NO:146 and 147, respectively. In one embodiment, the VH and the VL have the amino acid sequences of SEQ ID NO:148 and 149, respectively. In one embodiment, the VH and the VL have the amino acid sequences of SEQ ID NO:150 and 151, respectively. In one embodiment, the VH and the VL have the amino acid sequences of SEQ ID NO:152 and 153, respectively. In one embodiment, the VH and the VL have the amino acid sequences of SEQ ID NO:154 and 155, respectively. In one embodiment, the VH and the VL have the amino acid sequences of SEQ ID NO:156 and 157, respectively.

In one embodiment, the VH and the VL have the amino acid sequences of SEQ ID NO:158 and 159, respectively. In one embodiment, the VH and the VL have the amino acid sequences of SEQ ID NO:160 and 161, respectively. In one embodiment, the VH and the VL have the amino acid sequences of SEQ ID NO:162 and 163, respectively. In one embodiment, the VH and the VL have the amino acid sequences of SEQ ID NO:164 and 165, respectively. In one embodiment, the VH and the VL have the amino acid sequences of SEQ ID NO:166 and 167, respectively. In one embodiment, the VH and the VL have the amino acid sequences of SEQ ID NO:168 and 169, respectively. In one embodiment, the VH and the VL have the amino acid sequences of SEQ ID NO:170 and 171, respectively. In one embodiment, the VH and the VL have the amino acid sequences of SEQ ID NO:172 and 173, respectively.

In one embodiment, the VH and the VL have the amino acid sequences of SEQ ID NO:174 and 175, respectively. In one embodiment, the VH and the VL have the amino acid sequences of SEQ ID NO:176 and 177, respectively. In one embodiment, the VH and the VL have the amino acid sequences of SEQ ID NO:178 and 179, respectively. In one embodiment, the VH and the VL have the amino acid sequences of SEQ ID NO:180 and 181, respectively. In one embodiment, the VH and the VL have the amino acid sequences of SEQ ID NO:182 and 183, respectively. In one embodiment, the VH and the VL have the amino acid sequences of SEQ ID NO:184 and 185, respectively. In one embodiment, the VH and the VL have the amino acid sequences of SEQ ID NO:186 and 187, respectively. In one embodiment, the VH and the VL have the amino acid sequences of SEQ ID NO:188 and 189, respectively.

In one embodiment, the VH and the VL have the amino acid sequences of SEQ ID NO:190 and 191, respectively. In one embodiment, the VH and the VL have the amino acid sequences of SEQ ID NO:192 and 193, respectively. In one embodiment, the VH and the VL have the amino acid sequences of SEQ ID NO:194 and 195, respectively. In one embodiment, the VH and the VL have the amino acid sequences of SEQ ID NO:196 and 197, respectively. In one embodiment, the VH and the VL have the amino acid sequences of SEQ ID NO:198 and 199, respectively. In one embodiment, the VH and the VL have the amino acid sequences of SEQ ID NO:200 and 201, respectively. In one embodiment, the VH and the VL have the amino acid sequences of SEQ ID NO:202 and 203, respectively. In one embodiment, the VH and the VL have the amino acid sequences of SEQ ID NO:204 and 205, respectively.

In one embodiment, the VH and the VL have the amino acid sequences of SEQ ID NO:206 and 207, respectively. In one embodiment, the VH and the VL have the amino acid sequences of SEQ ID NO:208 and 209, respectively. In one embodiment, the VH and the VL have the amino acid sequences of SEQ ID NO:210 and 211, respectively. In one embodiment, the VH and the VL have the amino acid sequences of SEQ ID NO:212 and 213, respectively. In one embodiment, the VH and the VL have the amino acid sequences of SEQ ID NO:214 and 215, respectively. In one embodiment, the VH and the VL have the amino acid sequences of SEQ ID NO:216 and 217, respectively. In one embodiment, the VH and the VL have the amino acid sequences of SEQ ID NO:218 and 219, respectively. In one embodiment, the VH and the VL have the amino acid sequences of SEQ ID NO:220 and 221, respectively. In one embodiment, the VH and the VL have the amino acid sequences of SEQ ID NO:222 and 223, respectively.

In some embodiments, the VH CDR1 comprises an amino acid sequence of one of SEQ ID NOS:224-247, the VH CDR2 comprises an amino acid sequence of one of SEQ ID NOS:248-340, the VH CDR3 comprises an amino acid sequence of one of SEQ ID NOS:341-362, the VL CDR1 comprises an amino acid sequence of one of SEQ ID NOS:435-496, the VL CDR2 comprises an amino acid sequence of one of SEQ ID NOS:497-516, and the VL CDR3 comprises an amino acid sequence of one of SEQ ID NOS:517-594.

In some embodiments, the antibody may comprise a CDR that is at least 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical (or any range derivable therein) in sequence to SEQ ID NOS:224-594.

In certain aspects, a polypeptide can comprise 1, 2, and/or 3 CDRs from the variable heavy chain or variable light chain of SEQ ID NOS:4-223. The CDR may be one that has been determined by Kabat, IMGT, or Chothia. In further embodiments, a polypeptide may have CDRs that have 1, 2, and/or 3 amino acid changes (addition of 1 or 2 amino acids, deletions or 1 or 2 amino acids or substitution) with respect to these 1, 2, or 3 CDRs. In further embodiments, an antibody may be alternatively or additionally humanized in regions outside the CDR(s) and/or variable region(s). In some aspects, a polypeptide comprises additionally or alternatively, an amino acid sequence that is at least 60, 65, 70, 75, 80, 85, 90, 95, 96, 97, 98, 99, or 100% identical or homologous to the amino acid sequence of the variable region that is not a CDR sequence, i.e., the variable region framework.

From amino to carboxy terminus the CDRs are CDR1, CDR2, and CDR3. In some embodiments, a polypeptide may have CDRs that have 1, 2, and/or 3 amino acid changes (addition of 1 or 2 amino acids, deletions or 1 or 2 amino acids or substitution) with respect to CDR1, CDR2, or CDR3. In some embodiments, the CDRs of SEQ ID NOS:4-223 may further comprise 1, 2, 3, 4, 5, or 6 additional amino acids at the amino or carboxy terminus of the CDR; in some embodiments, the CDRs of SEQ ID NOS:4-223 may exclude 1, 2, 3, 4, 5, or 6 amino acids at the amino or carboxy terminus of the CDR. The additional amino acids may be from the heavy and/or light chain framework regions of SEQ ID NOS:4-223, that are shown as immediately adjacent to the CDRs. Accordingly, embodiments relate to antibodies comprising an HCDR1, HCDR2, HCDR3, LCDR1, LCDR2, and/or LCDR3 with at least or at most or exactly 1, 2, 3, 4, 5, 6 or 7 amino acids at the amino end of the CDR or at the carboxy end of the CDR, wherein the additional amino acids are the 1, 2, 3, 4, 5, 6, or 7 amino acids of SEQ ID NOS:4-223 that are shown as immediately adjacent to the CDRs. Other embodiments relate to antibodies comprising one or more CDRs, wherein the CDR is a fragment of SEQ ID NO:224-594 and wherein the fragment lacks 1, 2, 3, 4, or 5 amino acids from the amino or carboxy end of the CDR. In some embodiments, the CDR may lack one, 2, 3, 4, 5, 6, or 7 amino acids from the carboxy end and may further comprise 1, 2, 3, 4, 5, 6, 7, or 8 amino acids from the framework region of the amino end of the CDR. In some embodiments, the CDR may lack one, 2, 3, 4, 5, 6, or 7 amino acids from the amino end and may further comprise 1, 2, 3, 4, 5, 6, 7, or 8 amino acids from the framework region of the carboxy end of the CDR. In further embodiments, an antibody may be alternatively or additionally humanized in regions outside the CDR(s) and/or variable region(s). In some aspects, a polypeptide comprises additionally or alternatively, an amino acid sequence that is at least 60, 65, 70, 75, 80, 85, 90, 95, 96, 97, 98, 99, or 100% identical or homologous to the amino acid sequence of the variable region that is not a CDR sequence, i.e., the variable region framework.

In other embodiments, a polypeptide or protein comprises 1, 2, 3, 4, 5, and/or 6 CDRs from the either or both of the light and heavy variable regions of SEQ ID NOS:4-223, and 1, 2, 3, 4, 5, and/or 6 CDRs may have 1, 2, and/or 3 amino acid changes with respect to these CDRs. In some embodiments, parts or all of the antibody sequence outside the variable region have been humanized A protein may comprise one or more polypeptides. In some aspects, a protein may contain one or two polypeptides similar to a heavy chain polypeptide and/or 1 or 2 polypeptides similar to a light chain polypeptide.

In some embodiments, the antibody or antigen binding fragment comprises, comprises at least, or comprises at most 1, 2, 3, 4, 5, 6, 7, 8, 9, 10, 11, 12, 13, 14, 15, 16, 17, 18, 19, or 20 substitutions (or any derivable range therein) at amino acid position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, or 130 (or any range derivable therein) of the VH, VL, or CDR region identified in Tables 6, Table 7, the VH and VL region of SEQ ID NOS:4-223, or the CDR of SEQ ID NOS:224-594. In some embodiments, the amino acid at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, or 130 of the VH, VL, or CDR region identified in Tables 6, Table 7, the VH and VL region of SEQ ID NOS:4-223, or the CDR of SEQ ID NOS:224-594 is substituted with an alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine.

In some embodiments, the amino acid at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, or 130 of the VH, VL, or CDR region identified in Tables 6, Table 7, the VH and VL region of SEQ ID NOS:4-223, or the CDR of SEQ ID NOS:224-594 is substituted with a conservative amino acid.

In some embodiments, the amino acid at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, or 130 of the VH, VL, or CDR region identified in Tables 6, Table 7, the VH and VL region of SEQ ID NOS:4-223, or the CDR of SEQ ID NOS:224-594 is substituted with a non-conservative amino acid.

In some embodiments, the antibody or antigen binding fragment may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150 (or any derivable range therein) of any of SEQ ID NOS:4-594.

In some embodiments, the antibody or antigen binding fragment may comprise at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150, (or any derivable range therein) contiguous amino acids of any of SEQ ID NOS:4-594 that are at least, at most, or about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with any of SEQ ID NOS:4-594.

In some aspects there is a polypeptide, such as an antibody or antigen binding fragment starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, or 130 of any of SEQ ID NOS:4-594 and comprising at least, at most, or about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, or 130 (or any derivable range therein) contiguous amino acids or nucleotides of any of SEQ ID NOS:4-594.

Amino acid sequence variants of the disclosure can be substitutional, insertional, or deletion variants. In some embodiments, a variation in a polypeptide of the disclosure affects or affects at least or affects at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more noncontiguous or contiguous amino acids of the protein or polypeptide, as compared to a particular sequence, such as any of sequences disclosed herein. A variant can comprise an amino acid sequence that is at least 50%, 60%, 70%, 80%, or 90%, including all values and ranges there between, identical to any sequence provided or referenced herein. A variant can include 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the reference protein. Individual residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

One skilled in the art can determine suitable variants of polypeptides as set forth herein using well-known techniques. One skilled in the art may identify suitable areas of the molecule that may be changed without destroying activity by targeting regions not believed to be important for activity. The skilled artisan will also be able to identify amino acid residues and portions of the molecules that are conserved among similar proteins or polypeptides. In further embodiments, areas that may be important for biological activity or for structure may be subject to conservative amino acid substitutions without significantly altering the biological activity or without adversely affecting the protein or polypeptide structure.

In making such changes, the hydropathy index of amino acids may be considered. The hydropathy profile of a protein is calculated by assigning each amino acid a numerical value (“hydropathy index”) and then repetitively averaging these values along the peptide chain. Each amino acid has been assigned a value based on its hydrophobicity and charge characteristics. They are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (1.6); histidine (−3.2); glutamate (−3.5); glutamine (−3.5); aspartate (−3.5); asparagine (−3.5); lysine (−3.9); and arginine (−4.5). The importance of the hydropathy amino acid index in conferring interactive biologic function on a protein is generally understood in the art (Kyte et al., J. Mol. Biol. 157:105-131 (1982)). It is accepted that the relative hydropathic character of the amino acid contributes to the secondary structure of the resultant protein or polypeptide, which in turn defines the interaction of the protein or polypeptide with other molecules, for example, enzymes, substrates, receptors, DNA, antibodies, antigens, and others. It is also known that certain amino acids may be substituted for other amino acids having a similar hydropathy index or score, and still retain a similar biological activity. In making changes based upon the hydropathy index, in certain embodiments, the substitution of amino acids whose hydropathy indices are within ±2 is included. In some aspects of the invention, those that are within ±1 are included, and in other aspects of the invention, those within ±0.5 are included.

It also is understood in the art that the substitution of like amino acids can be effectively made based on hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. In certain embodiments, the greatest local average hydrophilicity of a protein, as governed by the hydrophilicity of its adjacent amino acids, correlates with its immunogenicity and antigen binding, that is, as a biological property of the protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, in certain embodiments, the substitution of amino acids whose hydrophilicity values are within ±2 are included, in other embodiments, those which are within ±1 are included, and in still other embodiments, those within ±0.5 are included. In some instances, one may also identify epitopes from primary amino acid sequences based on hydrophilicity. These regions are also referred to as “epitopic core regions.” It is understood that an amino acid can be substituted for another having a similar hydrophilicity value and still produce a biologically equivalent and immunologically equivalent protein.

Additionally, one skilled in the art can review structure-function studies identifying residues in similar polypeptides or proteins that are important for activity or structure. In view of such a comparison, one can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar proteins or polypeptides. In view of such information, one skilled in the art may predict the alignment of amino acid residues of an antibody with respect to its three-dimensional structure. One skilled in the art may choose not to make changes to amino acid residues predicted to be on the surface of the protein, since such residues may be involved in important interactions with other molecules. Moreover, one skilled in the art may generate test variants containing a single amino acid substitution at each desired amino acid residue. These variants can then be screened using standard assays for binding and/or activity, thus yielding information gathered from such routine experiments, which may allow one skilled in the art to determine the amino acid positions where further substitutions should be avoided either alone or in combination with other mutations. Various tools available to determine secondary structure can be found on the world wide web at expasy.org/proteomics/protein_structure.

In some embodiments of the invention, amino acid substitutions are made that: (1) reduce susceptibility to proteolysis, (2) reduce susceptibility to oxidation, (3) alter binding affinity for forming protein complexes, (4) alter ligand or antigen binding affinities, and/or (5) confer or modify other physicochemical or functional properties on such polypeptides. For example, single or multiple amino acid substitutions (in certain embodiments, conservative amino acid substitutions) may be made in the naturally occurring sequence. Substitutions can be made in that portion of the antibody that lies outside the domain(s) forming intermolecular contacts. In such embodiments, conservative amino acid substitutions can be used that do not substantially change the structural characteristics of the protein or polypeptide (e.g., one or more replacement amino acids that do not disrupt the secondary structure that characterizes the native antibody).

C. Methods of Identifying Activating Antibodies

The present disclosure further provides methods and agents useful for identify activating antibodies and fragments. As demonstrated in the experimental examples, an activating antibody or fragment is able to preferentially bind to a PLXDC1 or PLXDC2 protein in the presence of an activator, such as a small molecule compound of the present disclosure. The activator is contemplated to dissociate the homooligomer and make the PLXDC1 or PLXDC2 protein more accessible to the antibody. Once bound to the PLXDC1 or PLXDC2 protein, the antibody is able to keep the protein in the active (monomer) state.

In one embodiment, therefore, provided is a method for identifying an activator of a PLXDC protein, comprising contacting a candidate molecule with the PLXDC protein in the presence of a reference PLXDC activator, and detecting the binding affinity between the candidate molecule and the PLXDC protein, thereby identifying the candidate molecule as a PLXDC activator when the detected binding affinity is greater than a reference binding affinity between the candidate molecule and the PLXDC protein in the absence of the reference PLXDC activator. In some embodiments, the reference activator is a small molecule compound, such as those in Table 3. In some embodiments, the candidate molecule is an antibody, such as one from an antibody library.

### V. Nucleic Acids

Also provided herein are nucleic acid or polynucleotide molecules that encode the PLXDC1 and/or PLXDC2 receptors, antibodies, antigen binding fragments thereof and/or polypeptides that bind to PLXDC1 and/or PLXDC2 described herein. For example, the polynucleotide may encode an PLXDC1 and/or PLXDC2 protein or fragment thereof. The nucleic acids may be present, for example, in whole cells, in a cell lysate, or in a partially purified or substantially pure form.

In certain embodiments, nucleic acid sequences can exist in a variety of instances such as: isolated segments and recombinant vectors of incorporated sequences or recombinant polynucleotides encoding one or both chains of an antibody, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing described herein. Nucleic acids that encode the epitope to which certain of the antibodies provided herein are also provided. Nucleic acids encoding fusion proteins that include these peptides are also provided. The nucleic acids can be single-stranded or double-stranded and can comprise RNA and/or DNA nucleotides and artificial variants thereof (e.g., peptide nucleic acids).

The term “polynucleotide” refers to a nucleic acid molecule that either is recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein.

In certain embodiments, there are polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 90%, preferably 95% and above, identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, 5, 10, 15, 20, 25, 30, 35, 40, 45, 50, 75, 100, 125, 175, 200, 250, 300, 350, 400, 450, 500, 750, 1000, 1500, 3000, 5000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

A. Hybridization

The nucleic acids that hybridize to other nucleic acids under particular hybridization conditions. Methods for hybridizing nucleic acids are well known in the art. See, e.g., Current Protocols in Molecular Biology, John Wiley and Sons, N.Y. (1989), 6.3.1-6.3.6. As defined herein, a moderately stringent hybridization condition uses a prewashing solution containing 5x sodium chloride/sodium citrate (SSC), 0.5% SDS, 1.0 mM EDTA (pH 8.0), hybridization buffer of about 50% formamide, 6×SSC, and a hybridization temperature of 55° C. (or other similar hybridization solutions, such as one containing about 50% formamide, with a hybridization temperature of 42° C.), and washing conditions of 60° C. in 0.5×SSC, 0.1% SDS. A stringent hybridization condition hybridizes in 6×SSC at 45° C., followed by one or more washes in 0.1×SSC, 0.2% SDS at 68° C. Furthermore, one of skill in the art can manipulate the hybridization and/or washing conditions to increase or decrease the stringency of hybridization such that nucleic acids comprising nucleotide sequence that are at least 65%, at least 70%, at least 75%, at least 80%, at least 85%, at least 90%, at least 95%, at least 96%, at least 97%, at least 98% or at least 99% identical to each other typically remain hybridized to each other.

The parameters affecting the choice of hybridization conditions and guidance for devising suitable conditions are set forth by, for example, Sambrook, Fritsch, and Maniatis (Molecular Cloning: A Laboratory Manual, Cold Spring Harbor Laboratory Press, Cold Spring Harbor, N.Y., chapters 9 and 11 (1989); Current Protocols in Molecular Biology, Ausubel et al., eds., John Wiley and Sons, Inc., sections 2.10 and 6.3-6.4 (1995), both of which are herein incorporated by reference in their entirety for all purposes) and can be readily determined by those having ordinary skill in the art based on, for example, the length and/or base composition of the DNA.

B. Mutation

Changes can be introduced by mutation into a nucleic acid, thereby leading to changes in the amino acid sequence of a polypeptide (e.g., an antibody or antibody derivative) that it encodes. Mutations can be introduced using any technique known in the art. In one embodiment, one or more particular amino acid residues are changed using, for example, a site-directed mutagenesis protocol. In another embodiment, one or more randomly selected residues are changed using, for example, a random mutagenesis protocol. However it is made, a mutant polypeptide can be expressed and screened for a desired property.

Mutations can be introduced into a nucleic acid without significantly altering the biological activity of a polypeptide that it encodes. For example, one can make nucleotide substitutions leading to amino acid substitutions at non-essential amino acid residues. Alternatively, one or more mutations can be introduced into a nucleic acid that selectively changes the biological activity of a polypeptide that it encodes. See, eg., Romain Studer et al., Biochem. J. 449:581-594 (2013). For example, the mutation can quantitatively or qualitatively change the biological activity. Examples of quantitative changes include increasing, reducing or eliminating the activity. Examples of qualitative changes include altering the antigen specificity of an antibody.

C. Probes

In another aspect, nucleic acid molecules are suitable for use as primers or hybridization probes for the detection of nucleic acid sequences. A nucleic acid molecule can comprise only a portion of a nucleic acid sequence encoding a full-length polypeptide, for example, a fragment that can be used as a probe or primer or a fragment encoding an active portion of a given polypeptide.

In another embodiment, the nucleic acid molecules may be used as probes or PCR primers for specific antibody sequences. For instance, a nucleic acid molecule probe may be used in diagnostic methods or a nucleic acid molecule PCR primer may be used to amplify regions of DNA that could be used, inter alia, to isolate nucleic acid sequences for use in producing variable domains of antibodies. See, eg., Gaily Kivi et al., BMC Biotechnol. 16:2 (2016). In a preferred embodiment, the nucleic acid molecules are oligonucleotides. In a more preferred embodiment, the oligonucleotides are from highly variable regions of the heavy and light chains of the antibody of interest. In an even more preferred embodiment, the oligonucleotides encode all or part of one or more of the CDRs.

Probes based on the desired sequence of a nucleic acid can be used to detect the nucleic acid or similar nucleic acids, for example, transcripts encoding a polypeptide of interest. The probe can comprise a label group, e.g., a radioisotope, a fluorescent compound, an enzyme, or an enzyme co-factor. Such probes can be used to identify a cell that expresses the polypeptide.

### VI. Antibody Production

A. Antibody Production

Methods for preparing and characterizing antibodies for use in diagnostic and detection assays, for purification, and for use as therapeutics are well known in the art as disclosed in, for example, U.S. Pat. Nos. 4,011,308; 4,722,890; 4,016,043; 3,876,504; 3,770,380; and 4,372,745 (see, e.g., Antibodies: A Laboratory Manual, Cold Spring Harbor Laboratory, 1988; incorporated herein by reference). These antibodies may be polyclonal or monoclonal antibody preparations, monospecific antisera, human antibodies, hybrid or chimeric antibodies, such as humanized antibodies, altered antibodies, F(ab′)2 fragments, Fab fragments, Fv fragments, single-domain antibodies, dimeric or trimeric antibody fragment constructs, minibodies, or functional fragments thereof which bind to the antigen in question. In certain aspects, polypeptides, peptides, and proteins and immunogenic fragments thereof for use in various embodiments can also be synthesized in solution or on a solid support in accordance with conventional techniques. See, for example, Stewart and Young, (1984); Tarn et al, (1983); Merrifield, (1986); and Barany and Merrifield (1979), each incorporated herein by reference.

Briefly, a polyclonal antibody is prepared by immunizing an animal with an antigen or a portion thereof and collecting antisera from that immunized animal. The antigen may be altered compared to an antigen sequence found in nature. In some embodiments, a variant or altered antigenic peptide or polypeptide is employed to generate antibodies. Inocula are typically prepared by dispersing the antigenic composition in a physiologically tolerable diluent to form an aqueous composition. Antisera is subsequently collected by methods known in the arts, and the serum may be used as-is for various applications or else the desired antibody fraction may be purified by well-known methods, such as affinity chromatography (Harlow and Lane, Antibodies: A Laboratory Manual 1988).

Methods of making monoclonal antibodies are also well known in the art (Kohler and Milstein, 1975; Harlow and Lane, 1988, U.S. Pat. No. 4,196,265, herein incorporated by reference in its entirety for all purposes). Typically, this technique involves immunizing a suitable animal with a selected immunogenic composition, e.g., a purified or partially purified protein, polypeptide, peptide or domain Resulting antibody-producing B-cells from the immunized animal, or all dissociated splenocytes, are then induced to fuse with cells from an immortalized cell line to form hybridomas. Myeloma cell lines suited for use in hybridoma-producing fusion procedures preferably are non-antibody-producing and have high fusion efficiency and enzyme deficiencies that render then incapable of growing in certain selective media that support the growth of only the desired fused cells (hybridomas). Typically, the fusion partner includes a property that allows selection of the resulting hybridomas using specific media. For example, fusion partners can be hypoxanthine/aminopterin/thymidine (HAT)-sensitive. Methods for generating hybrids of antibody-producing spleen or lymph node cells and myeloma cells usually comprise mixing somatic cells with myeloma cells in the presence of an agent or agents (chemical or electrical) that promote the fusion of cell membranes. Next, selection of hybridomas can be performed by culturing the cells by single-clone dilution in microtiter plates, followed by testing the individual clonal supernatants (after about two to three weeks) for the desired reactivity. Fusion procedures for making hybridomas, immunization protocols, and techniques for isolation of immunized splenocytes for fusion are known in the art.

Other techniques for producing monoclonal antibodies include the viral or oncogenic transformation of B-lymphocytes, a molecular cloning approach may be used to generate a nucleic acid or polypeptide, the selected lymphocyte antibody method (SLAM) (see, e.g., Babcook et al., Proc. Natl. Acad. Sci. USA 93:7843-7848 (1996), the preparation of combinatorial immunoglobulin phagemid libraries from RNA isolated from the spleen of the immunized animal and selection of phagemids expressing appropriate antibodies, or producing a cell expressing an antibody from a genomic sequence of the cell comprising a modified immunoglobulin locus using Cre-mediated site-specific recombination (see, e.g., U.S. Pat. No. 6,091,001).

Monoclonal antibodies may be further purified using filtration, centrifugation, and various chromatographic methods such as HPLC or affinity chromatography. Monoclonal antibodies may be further screened or optimized for properties relating to specificity, avidity, half-life, immunogenicity, binding association, binding disassociation, or overall functional properties relative to being a treatment for infection. Thus, monoclonal antibodies may have alterations in the amino acid sequence of CDRs, including insertions, deletions, or substitutions with a conserved or non-conserved amino acid.

The immunogenicity of a particular immunogen composition can be enhanced by the use of non-specific stimulators of the immune response, known as adjuvants. Adjuvants that may be used in accordance with embodiments include, but are not limited to, IL-1, IL-2, IL-4, IL-7, IL-12, -interferon, GMCSP, BCG, aluminum hydroxide, MDP compounds, such as thur-MDP and nor-MDP, CGP (MTP-PE), lipid A, and monophosphoryl lipid A (MPL). Exemplary adjuvants may include complete Freund's adjuvant (a non-specific stimulator of the immune response containing killed Mycobacterium tuberculosis), incomplete Freund's adjuvants, and/or aluminum hydroxide adjuvant. In addition to adjuvants, it may be desirable to co-administer biologic response modifiers (BRM), such as but not limited to, Cimetidine (CIM; 1200 mg/d) (Smith/Kline, PA); low-dose Cyclophosphamide (CYP; 300 mg/m²) (Johnson/Mead, NJ), cytokines such as β-interferon, IL-2, or IL-12, or genes encoding proteins involved in immune helper functions, such as B-7. A phage-display system can be used to expand antibody molecule populations in vitro. Saiki, et al., Nature 324:163 (1986); Scharf et al., Science 233:1076 (1986); U.S. Pat. Nos. 4,683,195 and 4,683,202; Yang et al., J Mol Biol. 254:392 (1995); Barbas, III et al., Methods: Comp. Meth Enzymol. (1995) 8:94; Barbas, III et al., Proc Natl Acad Sci USA 88:7978 (1991).

B. Fully Human Antibody Production

Methods are available for making fully human antibodies. Using fully human antibodies can minimize the immunogenic and allergic responses that may be caused by administering non-human monoclonal antibodies to humans as therapeutic agents. In one embodiment, human antibodies may be produced in a non-human transgenic animal, e g, a transgenic mouse capable of producing multiple isotypes of human antibodies to protein (e.g., IgG, IgA, and/or IgE) by undergoing V-D-J recombination and isotype switching. Accordingly, this aspect applies to antibodies, antibody fragments, and pharmaceutical compositions thereof, but also non-human transgenic animals, B-cells, host cells, and hybridomas that produce monoclonal antibodies. Applications of humanized antibodies include, but are not limited to, detect a cell expressing an anticipated protein, either in vivo or in vitro, pharmaceutical preparations containing the antibodies of the present disclosure, and methods of treating disorders by administering the antibodies.

Fully human antibodies can be produced by immunizing transgenic animals (usually mice) that are capable of producing a repertoire of human antibodies in the absence of endogenous immunoglobulin production. Antigens for this purpose typically have six or more contiguous amino acids, and optionally are conjugated to a carrier, such as a hapten. See, for example, Jakobovits et al., Proc. Natl. Acad. Sci. USA 90:2551-2555 (1993); Jakobovits et al., Nature 362:255-258 (1993); Bruggermann et at, Year in Immunol. 7:33 (1993). In one example, transgenic animals are produced by incapacitating the endogenous mouse immunoglobulin loci encoding the mouse heavy and light immunoglobulin chains therein, and inserting into the mouse genome large fragments of human genome DNA containing loci that encode human heavy and light chain proteins. Partially modified animals, which have less than the full complement of human immunoglobulin loci, are then crossbred to obtain an animal having all of the desired immune system modifications. When administered an immunogen, these transgenic animals produce antibodies that are immunospecific for the immunogen but have human rather than murine amino acid sequences, including the variable regions. For further details of such methods, see, for example, International Patent Application Publication Nos. WO 96/33735 and WO 94/02602, which are hereby incorporated by reference in their entirety. Additional methods relating to transgenic mice for making human antibodies are described in U.S. Pat. Nos. 5,545,807; 6,713,610; 6,673,986; 6,162,963; 6,300,129; 6,255,458; 5,877,397; 5,874,299 and 5,545,806; in International Patent Application Publication Nos. WO 91/10741 and WO 90/04036; and in European Patent Nos. EP 546073B1 and EP 546073A1, all of which are hereby incorporated by reference in their entirety for all purposes.

The transgenic mice described above, referred to herein as “HuMAb” mice, contain a human immunoglobulin gene minilocus that encodes unrearranged human heavy (μ and ?) and ? light chain immunoglobulin sequences, together with targeted mutations that inactivate the endogenous μ and ? chain loci (Lonberg et al., Nature 368:856-859 (1994)). Accordingly, the mice exhibit reduced expression of mouse IgM or ? chains and in response to immunization, the introduced human heavy and light chain transgenes undergo class switching and somatic mutation to generate high affinity human IgG ? monoclonal antibodies (Lonberg et al., supra; Lonberg and Huszar, Intern. Ref. Immunol. 13:65-93 (1995); Harding and Lonberg, Ann. N.Y. Acad. Sci. 764:536-546 (1995)). The preparation of HuMAb mice is described in detail in Taylor et al., Nucl. Acids Res. 20:6287-6295 (1992); Chen et al., Int. Immunol. 5:647-656 (1993); Tuaillon et al., J. Immunol. 152:2912-2920 (1994); Lonberg et al., supra; Lonberg, Handbook of Exp. Pharmacol. 113:49-101 (1994); Taylor et al., Int. Immunol. 6:579-591 (1994); Lonberg and Huszar, Intern. Ref. Immunol. 13:65-93 (1995); Harding and Lonberg, Ann. N.Y. Acad. Sci. 764:536-546 (1995); Fishwild et al., Nat. Biotechnol. 14:845-851 (1996); the foregoing references are herein incorporated by reference in their entirety for all purposes. See further, U.S. Pat. Nos. 5,545,806; 5,569,825; 5,625,126; 5,633,425; 5,789,650; 5,877,397; 5,661,016; 5,814,318; 5,874,299; 5,770,429; and 5,545,807; as well as International Patent Application Publication Nos. WO 93/1227; WO 92/22646; and WO 92/03918, the disclosures of all of which are hereby incorporated by reference in their entirety for all purposes. Technologies utilized for producing human antibodies in these transgenic mice are disclosed also in WO 98/24893, and Mendez et al., Nat. Genetics 15:146-156 (1997), which are herein incorporated by reference. For example, the HCo7 and HCo12 transgenic mice strains can be used to generate human antibodies.

Using hybridoma technology, antigen-specific humanized monoclonal antibodies with the desired specificity can be produced and selected from the transgenic mice such as those described above. Such antibodies may be cloned and expressed using a suitable vector and host cell, or the antibodies can be harvested from cultured hybridoma cells. Fully human antibodies can also be derived from phage-display libraries (as disclosed in Hoogenboom et al., J. Mol. Biol. 227:381 (1991); and Marks et al., J. Mol. Biol. 222:581 (1991)). One such technique is described in International Patent Application Publication No. WO 99/10494 (herein incorporated by reference), which describes the isolation of high affinity and functional agonistic antibodies for MPL- and msk-receptors using such an approach.

C. Antibody Fragments Production

Antibody fragments that retain the ability to recognize the antigen of interest will also find use herein. A number of antibody fragments are known in the art that comprise antigen-binding sites capable of exhibiting immunological binding properties of an intact antibody molecule and can be subsequently modified by methods known in the arts. Functional fragments, including only the variable regions of the heavy and light chains, can also be produced using standard techniques such as recombinant production or preferential proteolytic cleavage of immunoglobulin molecules. These fragments are known as Fv. See, e.g., Inbar et al., Proc. Nat. Acad. Sci. USA 69:2659-2662 (1972); Hochman et al., Biochem. 15:2706-2710 (1976); and Ehrlich et al., Biochem. 19:4091-4096 (1980).

Single-chain variable fragments (scFvs) may be prepared by fusing DNA encoding a peptide linker between DNAs encoding the two variable domain polypeptides (VL and VH). scFvs can form antigen-binding monomers, or they can form multimers (e.g., dimers, trimers, or tetramers), depending on the length of a flexible linker between the two variable domains (Kortt et al., Prot. Eng. 10:423 (1997); Kort et al., Biomol. Eng. 18:95-108 (2001)). By combining different VL- and VH-comprising polypeptides, one can form multimeric scFvs that bind to different epitopes (Kriangkum et al., Biomol. Eng. 18:31-40 (2001)). Antigen-binding fragments are typically produced by recombinant DNA methods known to those skilled in the art. Although the two domains of the Fv fragment, VL and VH, are coded for by separate genes, they can be joined using recombinant methods by a synthetic linker that enables them to be made as a single chain polypeptide (known as single chain Fv (sFv or scFv); see e.g., Bird et al., Science 242:423-426 (1988); and Huston et al., Proc. Natl. Acad. Sci. USA 85:5879-5883 (1988). Design criteria include determining the appropriate length to span the distance between the C-terminus of one chain and the N-terminus of the other, wherein the linker is generally formed from small hydrophilic amino acid residues that do not tend to coil or form secondary structures. Suitable linkers generally comprise polypeptide chains of alternating sets of glycine and serine residues, and may include glutamic acid and lysine residues inserted to enhance solubility. Antigen-binding fragments are screened for utility in the same manner as intact antibodies. Such fragments include those obtained by amino-terminal and/or carboxy-terminal deletions, where the remaining amino acid sequence is substantially identical to the corresponding positions in the naturally occurring sequence deduced, for example, from a full-length cDNA sequence.

Antibodies may also be generated using peptide analogs of the epitopic determinants disclosed herein, which may consist of non-peptide compounds having properties analogous to those of the template peptide. These types of non-peptide compound are termed “peptide mimetics” or “peptidomimetics”. Fauchere, J. Adv. Drug Res. 15:29 (1986); Veber and Freidinger TINS p. 392 (1985); and Evans et al., J. Med. Chem. 30:1229 (1987). Liu et al. (2003) also describe “antibody like binding peptidomimetics” (ABiPs), which are peptides that act as pared-down antibodies and have certain advantages of longer serum half-life as well as less cumbersome synthesis methods. These analogs can be peptides, non-peptides or combinations of peptide and non-peptide regions. Fauchere, Adv. Drug Res. 15:29 (1986); Veber and Freidiner, TINS p. 392 (1985); and Evans et al., J. Med. Chem. 30:1229 (1987), which are incorporated herein by reference in their entirety for any purpose. Peptide mimetics that are structurally similar to therapeutically useful peptides may be used to produce a similar therapeutic or prophylactic effect. Such compounds are often developed with the aid of computerized molecular modeling. Generally, peptidomimetics of the disclosure are proteins that are structurally similar to an antibody displaying a desired biological activity, such as the ability to bind a protein, but have one or more peptide linkages optionally replaced by a linkage selected from: —CH₂NH—, —CH₂S—, —CH₂—CH₂—, —CH—CH-(cis and trans), —COCH2—, —CH(OH)CH₂—, and —CH₂SI—by methods well known in the art.

Systematic substitution of one or more amino acids of a consensus sequence with a D-amino acid of the same type (e.g., D-lysine in place of L-lysine) may be used in certain embodiments of the disclosure to generate more stable proteins. In addition, constrained peptides comprising a consensus sequence or a substantially identical consensus sequence variation may be generated by methods known in the art (Rizo and Gierasch, Ann. Rev. Biochem. 61:387 (1992), incorporated herein by reference), for example, by adding internal cysteine residues capable of forming intramolecular disulfide bridges which cyclize the peptide.

Once generated, a phage display library can be used to improve the immunological binding affinity of the Fab molecules using known techniques. See, e.g., Figini et al., J. Mol. Biol. 239:68 (1994). The coding sequences for the heavy and light chain portions of the Fab molecules selected from the phage display library can be isolated or synthesized and cloned into any suitable vector or replicon for expression. Any suitable expression system can be used.

### VII. Obtaining Encoded Antibodies and Polypeptides

In some aspects, there are nucleic acid molecule encoding antibody polypeptides (e.g., heavy or light chain, variable domain only, or full-length) or other peptides of the disclosure. These may be generated by methods known in the art, e.g., isolated from B cells of mice that have been immunized and isolated, phage display, expressed in any suitable recombinant expression system and allowed to assemble to form antibody molecules.

A. Expression

The nucleic acid molecules may be used to express large quantities of recombinant antibodies or to produce chimeric antibodies, single chain antibodies, immunoadhesins, diabodies, mutated antibodies, and other antibody derivatives. If the nucleic acid molecules are derived from a non-human, non-transgenic animal, the nucleic acid molecules may be used for antibody humanization.

B. Vectors

In some aspects, contemplated are expression vectors comprising a nucleic acid molecule encoding a polypeptide of the desired sequence or a portion thereof (e.g., a fragment containing one or more CDRs or one or more variable region domains). Expression vectors comprising the nucleic acid molecules may encode the heavy chain, light chain, or the antigen-binding portion thereof. In some aspects, expression vectors comprising nucleic acid molecules may encode fusion proteins, modified antibodies, antibody fragments, and probes thereof. In addition to control sequences that govern transcription and translation, vectors and expression vectors may contain nucleic acid sequences that serve other functions as well.

To express the antibodies, or antigen-binding fragments thereof, DNAs encoding partial or full-length light and heavy chains are inserted into expression vectors such that the gene area is operatively linked to transcriptional and translational control sequences. In some aspects, a vector that encodes a functionally complete human CH or CL immunoglobulin sequence with appropriate restriction sites engineered so that any VH or VL sequence can be easily inserted and expressed. Typically, expression vectors used in any of the host cells contain sequences for plasmid or virus maintenance and for cloning and expression of exogenous nucleotide sequences. Such sequences, collectively referred to as “flanking sequences” typically include one or more of the following operatively linked nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Such sequences and methods of using the same are well known in the art.

1. Expression Systems

Numerous expression systems exist that comprise at least a part or all of the expression vectors discussed above. Prokaryote- and/or eukaryote-based systems can be employed for use with an embodiment to produce nucleic acid sequences, or their cognate polypeptides, proteins and peptides. Commercially and widely available systems include in but are not limited to bacterial, mammalian, yeast, and insect cell systems. Different host cells have characteristic and specific mechanisms for the post-translational processing and modification of proteins. Appropriate cell lines or host systems can be chosen to ensure the correct modification and processing of the foreign protein expressed. Those skilled in the art are able to express a vector to produce a nucleic acid sequence or its cognate polypeptide, protein, or peptide using an appropriate expression system.

2. Methods of Gene Transfer

Suitable methods for nucleic acid delivery to effect expression of compositions are anticipated to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); or by PEG mediated transformation of protoplasts (Omirulleh et al., 1993; U.S. Pat. Nos. 4,684,611 and 4,952,500, each incorporated herein by reference); by desiccation/inhibition mediated DNA uptake (Potrykus et al., 1985). Other methods include viral transduction, such as gene transfer by lentiviral or retroviral transduction.

3. Host Cells

In another aspect, contemplated are the use of host cells into which a recombinant expression vector has been introduced. Antibodies can be expressed in a variety of cell types. An expression construct encoding an antibody can be transfected into cells according to a variety of methods known in the art. Vector DNA can be introduced into prokaryotic or eukaryotic cells via conventional transformation or transfection techniques. Some vectors may employ control sequences that allow it to be replicated and/or expressed in both prokaryotic and eukaryotic cells. In certain aspects, the antibody expression construct can be placed under control of a promoter that is linked to T-cell activation, such as one that is controlled by NFAT-1, which is a transcription factor that can be activated upon T-cell activation. Control of antibody expression allows T cells, such as tumor-targeting T cells, to sense their surroundings and perform real-time modulation of cytokine signaling, both in the T cells themselves and in surrounding endogenous immune cells. One of skill in the art would understand the conditions under which to incubate host cells to maintain them and to permit replication of a vector. Also understood and known are techniques and conditions that would allow large-scale production of vectors, as well as production of the nucleic acids encoded by vectors and their cognate polypeptides, proteins, or peptides.

For stable transfection of mammalian cells, it is known, depending upon the expression vector and transfection technique used, only a small fraction of cells may integrate the foreign DNA into their genome. In order to identify and select these integrants, a selectable marker (e.g., for resistance to antibiotics) is generally introduced into the host cells along with the gene of interest. Cells stably transfected with the introduced nucleic acid can be identified by drug selection (e.g., cells that have incorporated the selectable marker gene will survive, while the other cells die), among other methods known in the arts.

C. Isolation

The nucleic acid molecule encoding either or both of the entire heavy and light chains of an antibody or the variable regions thereof may be obtained from any source that produces antibodies. Methods of isolating mRNA encoding an antibody are well known in the art. See e.g., Sambrook et al., supra. The sequences of human heavy and light chain constant region genes are also known in the art. See, e.g., Kabat et al., 1991, supra. Nucleic acid molecules encoding the full-length heavy and/or light chains may then be expressed in a cell into which they have been introduced and the antibody isolated.

### VIII. Assay Methods

The present disclosure also provides methods for screening for and confirming therapeutic agents suitable for use in the present technology.

The instant inventors have developed ex vivo primary tumor angiogenesis assays to study the effects of therapeutic agents on tumor angiogenesis. The assays utilize an ex vivo primary tumor model generated from a tumor block dissected from a primary tumor tissue. The dissected tumor block, which may conform to preferred dimensions suitable for ex vivo growth, is embedded in a biological matrix, e.g., within hours, days, or weeks after removal from the organism, and cultured under conditions that promote growth of endothelial cells from the tumor block. Endothelial cells grow out of the tumor block and form a 2-dimensional or 3-dimensional outgrowth surrounding the tumor block, providing a suitable model system for drug testing. When treated with a candidate cancer therapeutic agent, death of the endothelial cells can effectively indicate the potential therapeutic efficacy of the agent on the tumor endothelial cells. In the same assay, the death of the tumor cells can be evaluated simultaneously to assess whether the reagent is specific to tumor endothelial cells or more generally cytotoxic.

Experimental examples of this ex vivo primary tumor angiogenesis model are provided in Example 1. Additional examples are described in U.S. Provisional Patent Application No. 62/809,857, filed on Feb. 25, 2019, the content of which is incorporated to the present disclosure by reference.

In certain aspects, provided herein are methods (e.g., in-vitro methods) of determining whether a test agent is a modulator of PLXDC1 and/or PLXDC2 (e.g., to select the agent as a potential therapeutic agent for the treatment of cancer), first by forming a test mixture comprising a test agent (e.g., a polynucleotide, a small molecule, an antibody or a peptide), incubating the test mixture with cells expressing PLXDC1 and/or PLXDC2 receptors and determining the level NF-κB activation. In some embodiments, the level of NF-κB activation may be determined, for example, by a cell culture based luciferase assay for receptor activation compared to a control mixture lacking the test agent. In some embodiments, a test agent that increases or triggers NF-κB activation compared to the NF-κB activation in a control mixture is a modulator of PLXDC1 and/or PLXDC2 receptor activity. In some embodiments, the test agent is an antibody, a peptide, a small molecule or a polynucleotide. In some embodiments, the test agent and/or the PLXDC1 and/or PLXDC2 receptor is linked to a detectable moiety. In some embodiments, the PLXDC1 and/or PLXDC2 receptor is ectopically expressed. In some embodiments, the control mixture is substantially identical to the test mixture except that the control mixture does not comprise a test agent. In some embodiments, the control mixture is substantially identical to the test mixture except that the control mixture comprises a placebo.

An activator of PLXDC1 and/or PLXDC2 described herein may be identified using an assay (e.g., an assay for screening candidates or test compounds which modulate PLXDC1 and/or PLXDC2). In some embodiments, an activator maybe identified using an assay and a library of test agents, wherein a test agent is an activator if the activator enriches a population of PLXDC1 and/or PLXDC2 receptors or increases the activity of PLXDC1 and/or PLXDC2 receptors on a tumor blood vessel. For example, the assay systems used to identify compounds that modulate (i.e., increase) the activity of PLXDC1 and/or PLXDC2 receptors may involve preparing a test mixture containing test agents under conditions and for a time sufficient to allow the test agents to modulate the PLXDC1 and/or PLXDC2 receptor. In order to test an agent for modulatory activity, the reaction mixture is prepared in the presence and absence of the test compound. The test compound can be initially included in the reaction mixture, or subsequently added at a later time. Control mixtures are incubated without the test compound or with a placebo. The increase in intracellular signaling pathways associated with PLXDC1 and/or PLXDC2 may be then be evaluated. An increase of intracellular signaling pathways associated with PLXDC1 and/or PLXDC2 in the test mixture, but less or no such change in the control mixture indicates the test compound is an activator of a PLXDC1 and/or PLXDC2 receptor. The assay for compounds that modulate PLXDC1 and/or PLXDC2 activity may be conducted with isolated test agent or pooled test agents. Pooled test agents comprise a test mixture with one or more test agents. The order of addition of test agents may be varied.

In some embodiments, the test agent is a member of a library of test agents. In some embodiments, assays used to identify agents useful in the methods include a reaction between PLXDC1 and/or PLXDC2 receptors and one or more assay components. The other components may be either a test compound (e.g. the agent), or a combination of test compounds. Agents identified via such assays, may be useful, for example, for preventing or treating cancer, or other PLXDC1 and/or PLXDC2 associated diseases. In some aspects, provided herein are methods of treating or preventing cancer in a subject comprising administering to the subject the test agent identified using the methods of identifying modulators of PLXDC1 and/or PLXDC2.

In some embodiments, the test agent (e.g. a polypeptide, a polynucleotide, a RNA molecule, or a small molecule) or PLXDC1 and/or PLXDC2 receptor is linked to a detectable moiety. As used herein, a detectable moiety may comprise a test agent or PLXDC1 and/or PLXDC2 receptor of the present disclosure linked to a distinct polypeptide or moiety to which it is not linked in nature. For example, the detectable moiety can be fused to the N-terminus or C-terminus of the test agent either directly, through a peptide bond, or indirectly through a chemical linker.

Agents useful in the methods of the present disclosure may be obtained from any available source, including systematic libraries of natural and/or synthetic compounds. Agents may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al., 1994, J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of agents may be presented in solution (e.g., Houghten, 1992, Biotechniques 13:412-421), or on beads (Lam, 1991, Nature 354:82-84), chips (Fodor, 1993, Nature 364:555-556), bacteria and/or spores, (Ladner, U.S. Pat. No. 5,223,409), plasmids (Cull et al, 1992, Proc Natl Acad Sci USA 89:1865-1869) or on phage (Scott and Smith, 1990, Science 249:386-390; Devlin, 1990, Science 249:404-406; Cwirla et al, 1990, Proc. Natl. Acad. Sci. 87:6378-6382; Felici, 1991, J. Mol. Biol. 222:301-310; Ladner, supra.).

Agents useful in the methods of the present disclosure may be identified, for example, using assays for screening candidate or test compounds which modulate the activity of PLXDC1 and/or PLXDC2 receptors. For example, candidate or test compounds can be screened for the ability to alter NF-κB signaling in a population of cells expressing PLXDC1 and/or PLXDC2. In some embodiments, the PLXDC1 and/or PLXDC2 are endogenously expressed. In some embodiments, the PLXDC1 and/or PLXDC2 are ectopically expressed. As described herein, the test compound is in a test mixture.

The basic principle of the assay systems used to identify compounds that modulate the activity of PLXDC1 and/or PLXDC2 receptors involves preparing a test mixture containing test agents under conditions and for a time sufficient to allow the test agents to modulate the PLXDC1 and/or PLXDC2 receptor. In order to test an agent for modulatory activity, the reaction mixture is prepared in the presence and absence of the test compound. The test compound can be initially included in the reaction mixture, or subsequently added at a later time. Control mixtures are incubated without the test compound or with a placebo. The NF-κB signaling may then tested. A change in NF-κB signaling, such as an increase in signaling, in the test mixture, but less or no such change in the control mixture indicates the test compound is a modulator of an PLXDC1 and/or PLXDC2 receptor. The assay for compounds that modulate PLXDC1 and/or PLXDC2 may be conducted with isolated test agent or pooled test agents. Pooled test agents comprise a test mixture with one or more test agents. The order of addition of test agents may be varied. Mixtures of chemicals or agents are added at consistent or varied concentration. Cells are then analyzed for fluorescence corresponding to NF-κB signaling.

### IX. Additional Therapies

A. Immunotherapy

In some embodiments, the methods comprise administration of a cancer immunotherapy. Cancer immunotherapy (sometimes called immuno-oncology, abbreviated I0) is the use of the immune system to treat cancer. Immunotherapies can be categorized as active, passive or hybrid (active and passive). These approaches exploit the fact that cancer cells often have molecules on their surface that can be detected by the immune system, known as tumour-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines Immumotherapies are known in the art, and some are described below.

1. Inhibition of co-stimulatory molecules

In some embodiments, the immunotherapy comprises an inhibitor of a co-stimulatory molecule. In some embodiments, the inhibitor comprises an inhibitor of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Inhibitors include inhibitory antibodies, polypeptides, compounds, and nucleic acids.

2. Dendritic cell therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

3. CAR-T cell therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signalling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Exemplary CAR-T therapies include Tisagenlecleucel (Kymriah) and Axicabtagene ciloleucel (Yescarta). In some embodiments, the CAR-T therapy targets CD19.

Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an exemplary interleukin cytokine therapy.

4. Adoptive T-cell therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumour death. [60]

Multiple ways of producing and obtaining tumour targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

B. Chemotherapies

In some embodiments, the additional therapy comprises a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-a), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). In some embodiments, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m2 to about 20 mg/m2 for 5 days every three weeks for a total of three courses being contemplated in certain embodiments. In some embodiments, the amount of cisplatin delivered to the cell and/or subject in conjunction with the construct comprising an Egr-1 promoter operably linked to a polynucleotide encoding the therapeutic polypeptide is less than the amount that would be delivered when using cisplatin alone.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Doxorubicin is absorbed poorly and is preferably administered intravenously. In certain embodiments, appropriate intravenous doses for an adult include about 60 mg/m2 to about 75 mg/m2 at about 21-day intervals or about 25 mg/m2 to about 30 mg/m2 on each of 2 or 3 successive days repeated at about 3 week to about 4 week intervals or about 20 mg/m2 once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

Gemcitabine diphosphate (GEMZAR®, Eli Lilly & Co., “gemcitabine”), another suitable chemotherapeutic agent, is recommended for treatment of advanced and metastatic pancreatic cancer, and will therefore be useful in the present disclosure for these cancers as well.

The amount of the chemotherapeutic agent delivered to the patient may be variable. In one suitable embodiment, the chemotherapeutic agent may be administered in an amount effective to cause arrest or regression of the cancer in a host, when the chemotherapy is administered with the construct. In other embodiments, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

C. Radiotherapy

In some embodiments, the additional therapy or prior therapy comprises radiation, such as ionizing radiation. As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

D. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### X. Treatments

The present disclosure provides methods and compositions for treating pathogenic blood vessel disorders such as diabetic retinopathy, age-related macular degeneration (AMD), retinopathy of prematurity, or cancer. The treatment can be through killing tumor blood vessels. In some embodiments, a tumor patient that can be suitably treated by the present technology expresses a plexin domain-containing protein (e.g., PLXDC1 or PLXDC2). The expression may be on a tumor blood epithelial cell.

As noted, the present technology not only can inhibit growth of new tumor blood vessels, but can also kill existing tumor blood vessels, thereby treating the tumor. In some embodiments, therefore, a tumor patient that can benefit from the present treatment is one that has a tumor that has undergone tumor angiogenesis. In some embodiments, the tumor comprises a vascularized tumor. In some embodiments, the tumor being treat has a diameter that is greater than about 0.1, 0.2, 0.3, 0.4, 0.5, 1, 1.5, 2, 2.5, 3, 4, 5, 6, 7, 8, 9 or 10 cm (or any derivable range therein). In some embodiments, the tumor already contains tumor blood vessels.

In some embodiments, the tumor does not have a known tumor surface marker as target for immunotherapy. In some embodiments, the tumor does not contain a mutant gene that serves as a target for tumor therapy. In some embodiments, the therapy of the present disclosure does not include inducing antibody-dependent cell-mediated cytotoxicity (ADCC). In some embodiments, a therapeutic agent of the present disclosure does not induce ADCC.

In some embodiments, the patient suffers from a cancer such as, polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyo sarcoma, colon carcinoma, pancreatic cancer, breast cancer, thyroid cancer, endometrial cancer, melanoma, prostate cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma and retinoblastoma.

In some embodiments, the methods described herein may be used to treat any cancerous or pre-cancerous tumor, such as a solid tumor. Cancers that may be treated by methods and compositions provided herein include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometrioid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; mammary paget's disease; acinar cell carcinoma; adenosquamous carcinoma; adenocarcinoma w/squamous metaplasia; malignant thymoma; malignant ovarian stromal tumor; malignant thecoma; malignant granulosa cell tumor; and malignant roblastoma; sertoli cell carcinoma; malignant leydig cell tumor; malignant lipid cell tumor; malignant paraganglioma; malignant extra-mammary paraganglioma; pheochromocytoma; glomangiosarcoma; malignant melanoma; amelanotic melanoma; superficial spreading melanoma; malignant melanoma in giant pigmented nevus; epithelioid cell melanoma; malignant blue nevus; sarcoma; fibrosarcoma; malignant fibrous histiocytoma; myxosarcoma; liposarcoma; leiomyosarcoma; rhabdomyosarcoma; embryonal rhabdomyosarcoma; alveolar rhabdomyosarcoma; stromal sarcoma; malignant mixed tumor; mullerian mixed tumor; nephroblastoma; hepatoblastoma; carcinosarcoma; malignant mesenchymoma; malignant brenner tumor; malignant phyllodes tumor; synovial sarcoma; malignant mesothelioma; dysgerminoma; embryonal carcinoma; malignant teratoma; malignant struma ovarii; choriocarcinoma; malignant mesonephroma; hemangiosarcoma; malignant hemangioendothelioma; kaposi's sarcoma; malignant hemangiopericytoma; lymphangiosarcoma; osteosarcoma; juxtacortical osteosarcoma; chondrosarcoma; malignant chondroblastoma; mesenchymal chondrosarcoma; giant cell tumor of bone; ewing's sarcoma; malignant odontogenic tumor; ameloblastic odontosarcoma; malignant ameloblastoma; ameloblastic fibrosarcoma; malignant pinealoma; chordoma; malignant glioma; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; malignant meningioma; neurofibrosarcoma; malignant neurilemmoma; malignant granular cell tumor; malignant lymphoma; Hodgkin's disease; Hodgkin's lymphoma; paragranuloma; small lymphocytic malignant lymphoma; diffuse large cell malignant lymphoma; follicular malignant lymphoma; mycosis fungoides; other specified non-Hodgkin's lymphomas; malignant histiocytosis; multiple myeloma; mast cell sarcoma or immunoproliferative small intestinal disease.

In some embodiments, the subject has cancer, preferably comprising a solid tumor. An agent disclosed herein may be administered locally to the tumor. In some embodiments, the tumor is an adenocarcinoma, an adrenal tumor, an anal tumor, a bile duct tumor, a bladder tumor, a bone tumor, a blood born tumor, a brain/CNS tumor, a breast tumor, a cervical tumor, a colorectal tumor, an endometrial tumor, an esophageal tumor, an Ewing tumor, an eye tumor, a gallbladder tumor, a gastrointestinal, a kidney tumor, a laryngeal or hypopharyngreal tumor, a liver tumor, a lung tumor, a mesothelioma tumor, a multiple myeloma tumor, a muscle tumor, a nasopharyngeal tumor, a neuroblastoma, an oral tumor, an osteosarcoma, an ovarian tumor, a pancreatic tumor, a penile tumor, a pituitary tumor, a primary tumor, a prostate tumor, a retinoblastoma, a Rhabdomyosarcoma, a salivary gland tumor, a soft tissue sarcoma, a melanoma, a metastatic tumor, a basal cell carcinoma, a Merkel cell tumor, a testicular tumor, a thymus tumor, a thyroid tumor, a uterine tumor, a vaginal tumor, a vulvar tumor, or a Wilms tumor. In some embodiments, a compound and/or composition described herein may be administered parenterally, at or near the site of a tumor, or distant from the site of the tumor.

Actual dosage levels of the active ingredients in the pharmaceutical compositions may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular agent employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could prescribe and/or administer doses of the compounds employed in the pharmaceutical composition at levels lower than that required in order to achieve the desired therapeutic effect and gradually increase the dosage until the desired effect is achieved.

The administration of one or more compounds as described herein may result in at least a 10% decrease (e.g., at least 15%, 20%, 25%, 30%, 35%, 40%, 45%, 50%, 55%, 60%, 65%, 70%, 75%, 80%, 85%, 90% or even 100% decrease in one or more symptoms of a disease or condition, such as a decrease in tumor size.

Compounds described herein can be used in methods for agonizing Pigment-Epithelium-Derived Factor (PEDF) receptors. Compounds described herein can also be used in methods for inhibiting angiogenesis.

The PEDF receptors have been identified as two homologous membrane proteins called plexin domain containing 1 (PLXDC1) and plexin domain containing 2 (PLXDC2). They belong to a new type of cell-surface receptors and are the only proteins that are known to confer cell-surface binding to PEDF and to transduce PEDF signal into the target cells. Consistent with the ability of PEDF to suppress pathogenic angiogenesis in blinding diseases and in cancer without affecting healthy blood vessels, the PEDF receptors are highly expressed in pathogenic blood vessels in many diseases, including tumor blood vessels and diabetic retinopathy. The PEDF receptors are not detected in healthy blood vessels. One of the PEDF receptors (TEM7, PLXDC1) was well studied in the past as a tumor endothelial marker that is enriched in tumor blood vessels of diverse types of human cancer including colon, liver, lung, breast, pancreatic, brain, bladder, ovarian, kidney, esophagus, gastric and endometrial cancer and Kaposi sarcoma, liposarcoma and synovial sarcoma. In blinding diseases, PEDF receptor TEM7 (PLXDC1) is highly enriched in the pathogenic blood vessels of diabetic retinopathy, retinal occlusive vascular disease, retinopathy of prematurity, and choroidal neovascularization (pathogenic angiogenesis in AMD). This is consistent with the role of PEDF in suppressing pathogenic angiogenesis in these diseases without affecting healthy blood vessels.

The compounds and methods described herein can therefore be used in treating disease mediated by PEDF receptors or associated with angiogenesis, such as cancer, retinal occlusive vascular disease, retinopathy of prematurity, diabetic retinopathy, and age-related macular degeneration.

In certain embodiments, provided herein are methods for agonizing Pigment-Epithelium-Derived Factor (PEDF) receptors in a patient in need thereof comprising administering to said patient a therapeutically effective amount of a compound of the disclosure, or a pharmaceutically acceptable salt or prodrug thereof.

In certain embodiments, provided herein are methods for inhibiting angiogenesis in a patient in need thereof comprising administering to said patient a therapeutically effective amount of a compound of the disclosure, or a pharmaceutically acceptable salt or prodrug thereof. In certain embodiments, the angiogenesis is pathogenic angiogenesis.

Also provided herein are methods for treating a disease or disorder mediated by PEDF receptors in a patient in need thereof comprising administering to said patient a therapeutically effective amount of a compound of the disclosure, or a pharmaceutically acceptable salt or prodrug thereof.

Also provided herein are methods for treating a disease or disorder associated with angiogenesis in a patient in need thereof comprising administering to said patient a therapeutically effective amount of a compound of the disclosure, or a pharmaceutically acceptable salt or prodrug thereof.

Also provided herein are methods for treating a disease or disorder selected from a cancer, retinal occlusive vascular disease, retinopathy of prematurity, diabetic retinopathy, and age-related macular degeneration comprising administering to said patient a therapeutically effective amount of a compound of the disclosure, or a pharmaceutically acceptable salt or prodrug thereof. In certain embodiments, the cancer is selected from colon, liver, lung, breast, pancreatic, brain, bladder, ovarian, kidney, esophagus, gastric and endometrial cancer and Kaposi sarcoma, liposarcoma and synovial sarcoma. In certain embodiments, the disease is a blinding disease. In certain embodiments, the disease is diabetic retinopathy, retinal occlusive vascular disease, retinopathy of prematurity, or choroidal neovascularization (pathogenic angiogenesis in AMD).

In certain embodiments, provided herein is use of a compound of the disclosure, or a pharmaceutically acceptable salt prodrug thereof in a method for agonizing Pigment-Epithelium-Derived Factor (PEDF) receptors in a patient in need thereof comprising administering to said patient a therapeutically effective amount of the compound, or a pharmaceutically acceptable salt prodrug thereof.

In certain embodiments, provided herein is use of a compound of the disclosure, or a pharmaceutically acceptable salt prodrug thereof in a method for inhibiting angiogenesis in a patient in need thereof comprising administering to said patient a therapeutically effective amount of the compound, or a pharmaceutically acceptable salt or prodrug thereof. In certain embodiments, the angiogenesis is pathogenic angiogenesis.

Also provided herein is use of a compound of the disclosure, or a pharmaceutically acceptable salt or prodrug thereof in a method for treating a disease or disorder mediated by PEDF receptors in a patient in need thereof comprising administering to said patient a therapeutically effective amount of the compound, or a pharmaceutically acceptable salt or prodrug thereof.

Also provided herein is use of a compound of the disclosure, or a pharmaceutically acceptable salt or prodrug thereof in a method for treating a disease or disorder associated with angiogenesis in a patient in need thereof comprising administering to said patient a therapeutically effective amount of the compound, or a pharmaceutically acceptable salt or prodrug thereof.

Also provided herein is use of a compound of the disclosure, or a pharmaceutically acceptable salt or prodrug thereof in a method for treating a disease or disorder selected from a cancer, retinal occlusive vascular disease, retinopathy of prematurity, diabetic retinopathy, and age-related macular degeneration comprising administering to said patient a therapeutically effective amount of the compound, or a pharmaceutically acceptable salt or prodrug thereof. In certain embodiments, the cancer is selected from colon, liver, lung, breast, pancreatic, brain, bladder, ovarian, kidney, esophagus, gastric and endometrial cancer and Kaposi sarcoma, liposarcoma and synovial sarcoma. In certain embodiments, the disease is a blinding disease. In certain embodiments, the disease is diabetic retinopathy, retinal occlusive vascular disease, retinopathy of prematurity, or choroidal neovascularization (pathogenic angiogenesis in AMD).

In certain embodiments, provided herein is use of a compound of the disclosure, or a pharmaceutically acceptable salt or prodrug thereof in the manufacture of a medicament for agonizing Pigment-Epithelium-Derived Factor (PEDF) receptors in a patient in need thereof.

In certain embodiments, provided herein is use of a compound of the disclosure, or a pharmaceutically acceptable salt or prodrug thereof in the manufacture of a medicament for inhibiting angiogenesis in a patient in need thereof. In certain embodiments, the angiogenesis is pathogenic angiogenesis.

Also provided herein is use of a compound of the disclosure, or a pharmaceutically acceptable salt or prodrug thereof in the manufacture of a medicament for treating a disease or disorder mediated by PEDF receptors in a patient in need thereof.

Also provided herein is use of a compound of the disclosure, or a pharmaceutically acceptable salt or prodrug thereof in the manufacture of a medicament for treating a disease or disorder associated with angiogenesis in a patient in need thereof.

Also provided herein is use of a compound of the disclosure, or a pharmaceutically acceptable salt or prodrug thereof in the manufacture of a medicament for treating a disease or disorder selected from a cancer, retinal occlusive vascular disease, retinopathy of prematurity, diabetic retinopathy, and age-related macular degeneration. In certain embodiments, the cancer is selected from colon, liver, lung, breast, pancreatic, brain, bladder, ovarian, kidney, esophagus, gastric and endometrial cancer and Kaposi sarcoma, liposarcoma and synovial sarcoma. In certain embodiments, the disease is a blinding disease. In certain embodiments, the disease is diabetic retinopathy, retinal occlusive vascular disease, retinopathy of prematurity, or choroidal neovascularization (pathogenic angiogenesis in AMD)

### XI. Administration of Therapeutic Compositions

The present disclosure also provides pharmaceutical compositions. In certain embodiments provided herein is a composition, e.g., a pharmaceutical composition, containing at least one antibody, small molecule, polynucleotide or polypeptide capable of modulating an PLXDC1 and/or PLXDC2 receptor described herein, formulated together with a pharmaceutically acceptable carrier. In some embodiments, the composition includes a combination of multiple (e.g., two or more) agents.

The therapy provided herein may comprise administration of a combination of therapeutic agents, such as a first cancer therapy and a second cancer therapy. The therapies may be administered in any suitable manner known in the art. For example, the first and second cancer treatment may be administered sequentially (at different times) or concurrently (at the same time). In some embodiments, the first and second cancer treatments are administered in a separate composition. In some embodiments, the first and second cancer treatments are in the same composition.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some embodiments, the cancer therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### XII. Kits

Certain aspects of the present disclosure also concern kits containing compositions of the disclosure or compositions to implement methods of the disclosure. In some embodiments, kits can be used to evaluate one or more biomarkers. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein. In some embodiments, there are kits for evaluating biomarker activity in a cell.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1x, 2x, 5x, 10x, or 20x or more.

Kits for using probes, synthetic nucleic acids, nonsynthetic nucleic acids, and/or inhibitors of the disclosure for prognostic or diagnostic applications are included as part of the disclosure. Specifically contemplated are any such molecules corresponding to any biomarker identified herein, which includes nucleic acid primers/primer sets and probes that are identical to or complementary to all or part of a biomarker, which may include noncoding sequences of the biomarker, as well as coding sequences of the biomarker.

It is contemplated that any method or composition described herein can be implemented with respect to any other method or composition described herein and that different embodiments may be combined. The claims originally filed are contemplated to cover claims that are multiply dependent on any filed claim or combination of filed claims.

Any embodiment of the disclosure involving specific biomarker by name is contemplated also to cover embodiments involving biomarkers whose sequences are at least 80, 81, 82, 83, 84, 85, 86, 87, 88, 89, 90, 91, 92, 93, 94, 95, 96, 97, 98, 99% identical to the mature sequence of the specified nucleic acid.

Embodiments of the disclosure include kits for analysis of a pathological sample by assessing biomarker profile for a sample comprising, in suitable container means, two or more biomarker probes, wherein the biomarker probes detect one or more of the biomarkers identified herein. The kit can further comprise reagents for labeling nucleic acids in the sample. The kit may also include labeling reagents, including at least one of amine-modified nucleotide, poly(A) polymerase, and poly(A) polymerase buffer. Labeling reagents can include an amine-reactive dye.

Provided herein are also kits that include a compound of the disclosure, or a pharmaceutically acceptable salt or prodrug thereof, and suitable packaging. In certain embodiments, a kit further includes instructions for use. In one aspect, a kit includes a compound of the disclosure, or a pharmaceutically acceptable salt or prodrug thereof, and a label and/or instructions for use of the compounds in the treatment of the indications, including the diseases or conditions, described herein.

Provided herein are also articles of manufacture that include a compound described herein or a pharmaceutically acceptable salt or prodrug thereof in a suitable container. The container may be a vial, jar, ampoule, preloaded syringe and intravenous bag.

### XIII. Synthesis of the Compounds

The compounds may be prepared using the methods disclosed herein and routine modifications thereof, which will be apparent given the disclosure herein and methods well known in the art. Conventional and well-known synthetic methods may be used in addition to the teachings herein. The synthesis of typical compounds described herein may be accomplished as described in the following examples. If available, reagents and starting materials may be purchased commercially, e.g., from Sigma Aldrich or other chemical suppliers.

It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in Wuts, P. G. M., Greene, T. W., & Greene, T. W. (2006). Greene's protective groups in organic synthesis. Hoboken, N.J., Wiley-Interscience, and references cited therein.

Furthermore, the compounds of this disclosure may contain one or more chiral centers. Accordingly, if desired, such compounds can be prepared or isolated as pure stereoisomers, i.e., as individual enantiomers or diastereomers or as stereoisomer-enriched mixtures. All such stereoisomers (and enriched mixtures) are included within the scope of this disclosure, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents, and the like.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chemce or Sigma (St. Louis, Mo., USA). Others may be prepared by procedures or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989) organic Reactions, Volumes 1-40 (John Wiley, and Sons, 1991), March's Advanced Organic Chemistry, (John Wiley, and Sons, 5th Edition, 2001), and Larock's Comprehensive Organic Transformations (VCH Publishers Inc., 1989).

A. General Synthesis

Scheme I illustrates a general method which can be employed for the synthesis of compounds described herein.

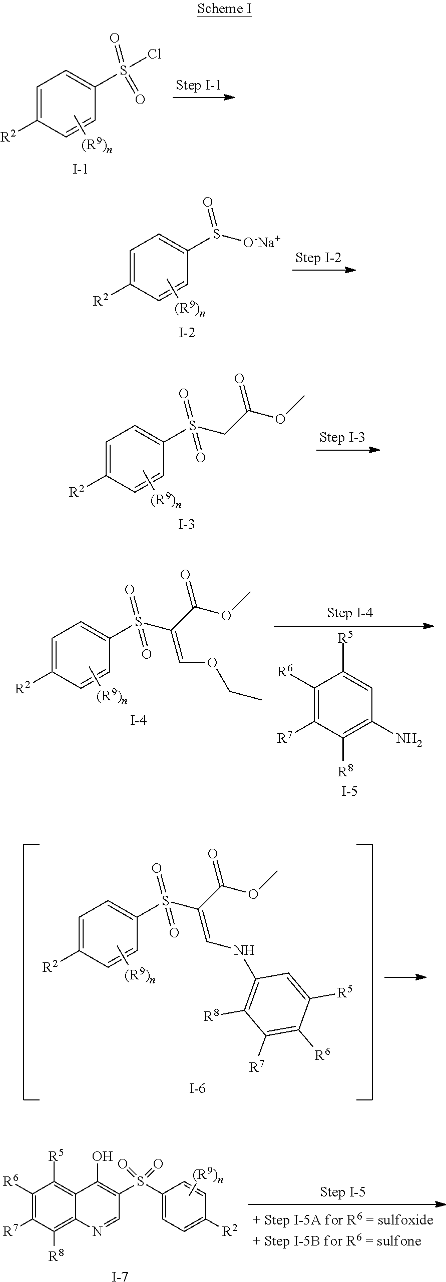

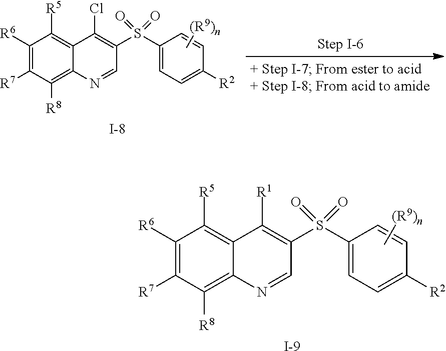

Referring to Scheme I, wherein R¹, R², R⁵, R⁶, R⁷, R⁸, R⁹ and n are as described herein, appropriate starting materials and reagents, such as compound I-1, can be purchased or prepared by methods known to one of skill in the art or by methods described herein, such as in Scheme II.

Step I-1: Compound I-1 reacts with a sodium salt (e.g., Na₂SO₃) or a sodium base (e.g., NaHCO₃), or a mixture thereof in an aqueous solution to give Compound 1-2. The reaction can be carried out under heating conditions, such as at a temperature of from about 60° C. to about 100° C. Examples of the reaction are illustrated in Example 1, Step I-2, and Example 6, Step 6-2.

Step I-2: Compound 1-2 reacts with Lg¹-CH₂C(O)OCH₃, wherein Lg¹ is a leaving group, such as Cl or Br, to provide 1-3. The reaction can be carried out in a solvent, such as DMF, under heating conditions, such as at a temperature of from about 60° C. to about 100° C. An example of the reaction is illustrated in Example 1, Step 1-3.

Step I-3: Compound 1-3 reacts with triethyl orthoformate and acetic anhydride to provide Compound 1-4. The reaction can be carried out under reflex conditions. An example of the reaction is illustrated in Example 1, Step 1-4.

Step I-4: Compound 1-4 reacts with amino Compound 1-5 to provide Compound 1-6. The reaction can be carried out in a solvent, such as diphenyl ether, under heating conditions, such as at a temperature of from about 100° C. to about 150° C. Compound 1-6 then cyclizes to Compound 1-7. The cyclization reaction can be carried out in a solvent, such as diphenyl ether, under reflux conditions. An example of the reaction is illustrated in Example 1, Step 1-5.

Step I-5: Compound 1-7 reacts with POCl₃ to give Compound 1-8. The reaction can be carried out under reflux conditions. Optionally, a solvent, such as DMF, may be used. Examples of the reaction are illustrated in Example 1, Step 1-6, and Example 3.

Step I-5A: When R⁶ of Compound 1-8 is —SR¹⁶ (wherein R¹⁶ is as defined herein), the —SR¹⁶ group can be oxidized to —S(O)R¹⁶ using 1 eq. of an oxidizing reagent, such as mCPBA, to give Compound 1-8 wherein R⁶ is —S(O)R¹⁶. The reaction can be carried out at a low temperature of below 0° C., such as about −20° C. An example of the reaction is illustrated in Example 8, Step 8-1.

Step I-5B: When R⁶ of Compound 1-8 is —SR¹⁶ (wherein R¹⁶ is as defined herein), the —SR¹⁶ group can be oxidized to —S(O)₂R¹⁶ by adding an excess amount of an oxidizing reagent, such as 2. eq. of mCPBA, to give Compound 1-8 wherein R⁶ is —S(O)₂R¹⁶. The reaction can be carried out at a temperature of about 0° C. An example of the reaction is illustrated in Example 9.

Step I-6: Compound 1-8 reacts with Compound R¹—H to give Compound 1-9. The reaction can be carried out in a solvent, such as 1,4-dioxane and DMF, optionally with heating, such as at a temperature of about 30° C. to reflex. In certain embodiments, a base such as NaH can be added to deprotonate Compound R¹—H before reaction with Compound 1-8. Examples of 1-6 are illustrated in Example 1, Step 1-7 (I-6A), Example 2 (I-6B), and Example 5 (I-6C).

Step I-7: When R⁶ of Compound 1-9 is an ester —C(O)OR¹⁵ (wherein R¹⁵ is as defined herein but is not H), —C(O)OR¹⁵ can be hydrolyzed to —C(O)OH with a base such as LiOH, in an aqueous solution, to give Compound 1-9 wherein R⁶ is —C(O)OH. An example of the reaction is illustrated in Example 3, conversion of Compound 63 to Compound 66. Similarly, an ester group at other positions of a compound can be hydrolyzed to an acid group.

Step I-8: Compound 1-9 wherein R⁶ is —C(O)OH can be converted to Compound 1-9 wherein R⁶ is —C(O)NR¹⁵R¹⁵ by reacting with an amine HNR¹⁵R¹⁵ under amide coupling reaction conditions. Amide coupling reaction conditions can include a solvent, such as NMP, DMF, DCM, a coupling reagent, such as EDCI, optionally an additional agent, such as HOBt, and optionally a base, such as triethylamine. The reaction can be carried out at about 0° C. to room temperature. An example of the reaction is illustrated in Example 7.

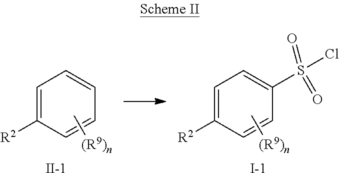

Scheme II shows a method of preparing starting material I-1 used in Scheme I from compound II-1.

In certain embodiments, Compound II-1 reacts with phosphorus oxychloride and concentrated sulfuric acid to give Compound I-1 (II-A). The reaction may be carried out at an elevated temperature, such as about 60° C. to about 100° C. An example of the reaction is illustrated in Example 1, Step 1-1.

In certain embodiments, Compound II-1 reacts with chlorosulfonic acid to give Compound I-1 (II-B). The reaction may be carried out at a low temperature, such as about −10° C. to about 10° C. An example of the reaction is illustrated in Example 6, Step 6-1.

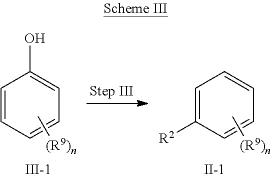

Scheme III shows a method of preparing starting material II-1. Phenol Compound II-1 react with Lg²-R¹⁸ to give Compound 11-2, wherein R² is —OR¹⁸, R⁹ and R¹⁸ are as defined herein, and Lg² is a leaving group, such as a halo. The reaction can be carried out in a solvent, such as acetone in the presence of a base such as K₂CO₃, and a phase transfer catalyst, such as tetra-n-butylammonium iodide. An example of the reaction is illustrated in Example 10.

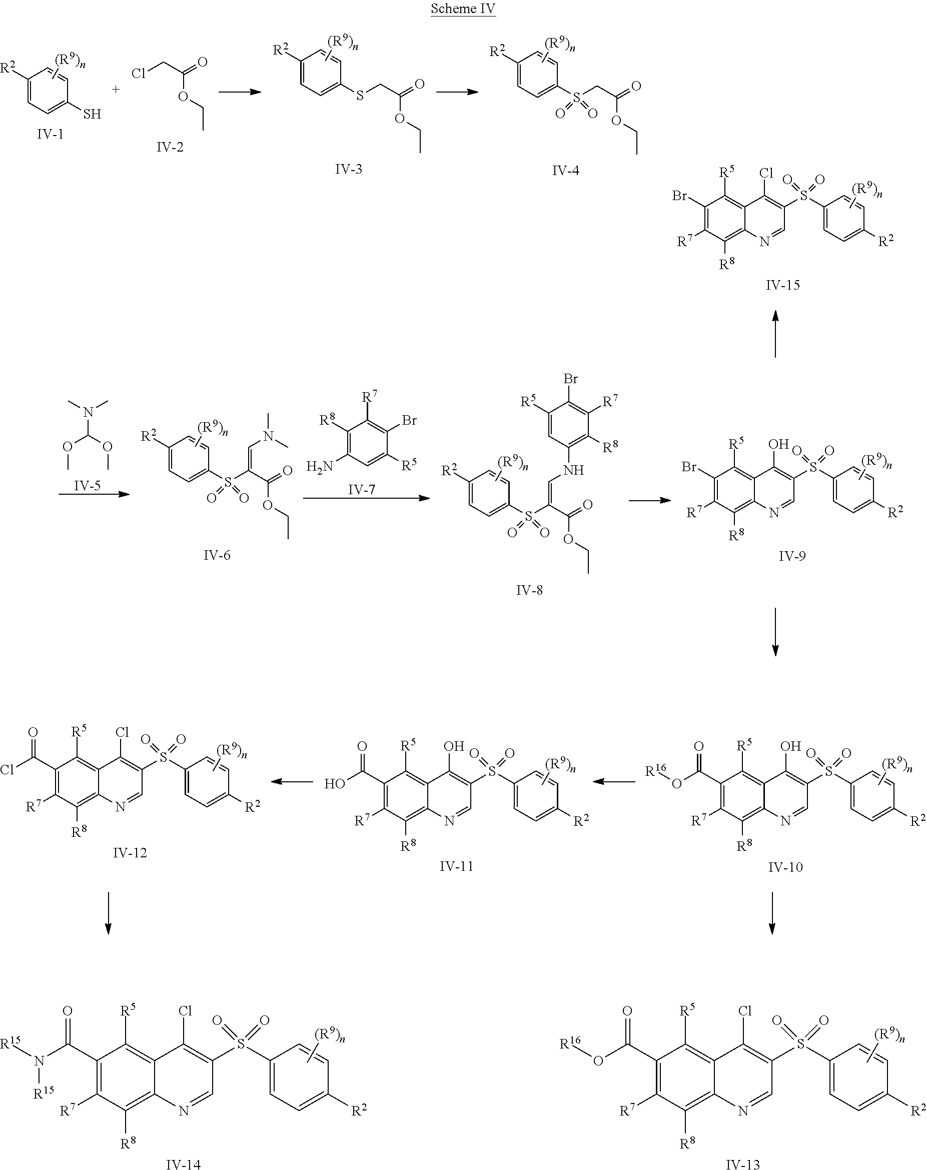

Scheme IV shows a method of preparing Intermediates such as IV-13, IV-14, and IV-15, wherein R², R⁵, R⁶, R⁷, R⁸, R⁹, R¹⁵, R¹⁶, and n are as defined herein. An example of the method is illustrated in Example 18.

### XIV. Biological Examples

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1. Killing Pathogenic Blood Vessels by Targeting PLXDC1/PLXDC2

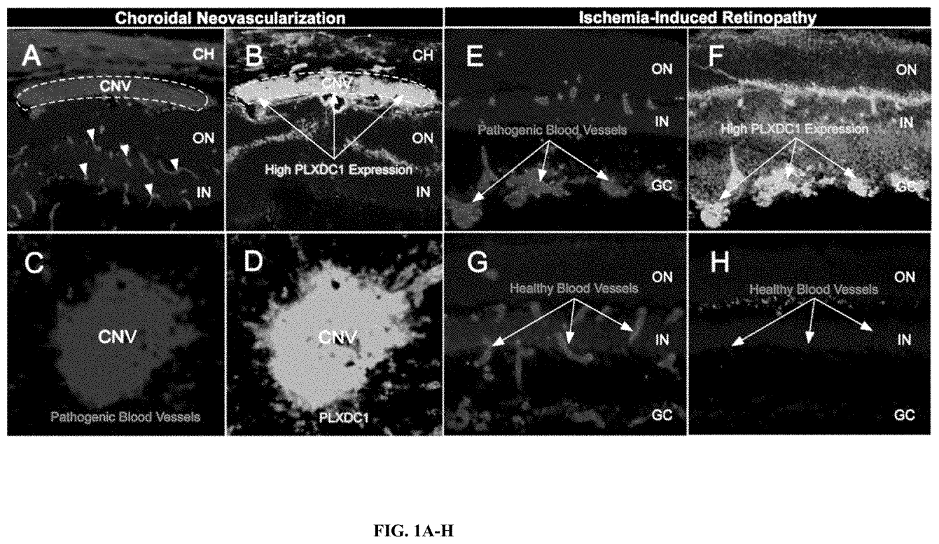

Pathogenic angiogenesis plays a key role in several major human diseases (Carmeliet, 2005). In addition to tumor growth and metastasis, angiogenesis is a major pathogenic driving force in several blinding diseases including diabetic retinopathy, age-related macular degeneration (AMD), and retinopathy of prematurity. AMD and diabetic retinopathy are the leading causes of blindness in the elderly and populations at the working age in the United States, respectively. Retinopathy of prematurity is a common reason that causes the loss of vision for newborn babies. Pathogenic blood vessels are blood vessels that exist in the diseased states such as tumor blood vessels in tumors and new blood vessels in AMD or diabetic retinopathy that are distinct from healthy blood vessels in the eye. As demonstrated in FIG. 1, PLXDC1 expression was highly enriched in pathogenic blood vessels in a mouse model of CNV (laser-induced choroidal neovascularization (CNV) (FIG. 1A-D), in pathogenic blood vessels in a mouse model of ischemia-induced retinopathy, but not in blood vessels of healthy retina (FIG. 1E-H).

Pathogenic blood vessels differ from healthy blood vessels not only in tissue location and health state, but also in function. Pathogenic blood vessels drive pathogenic processes. For example, tumor blood vessels drive tumor growth and supply tumor with oxygen and nutrients that are essential for its survival. Choroidal neovascularization, the pathogenic blood vessels in AMD, cause blindness due to leakage that kills healthy neurons. While there are current therapeutic strategies for inhibiting the growth of new blood vessels, such as anti-angiogenesis therapies, there are no known strategies for destroying already existing pathogenic blood vessels. The binding agents of the disclosure can kill existing pathogenic blood vessels, thus providing an improvement over existing anti-angiogenic therapies.

Two markers of pathogenic blood vessels, PLXDC1 (TEM7) and PLXDC2, are highly specifically expressed in the tumor blood vessels of diverse types of cancer (Beaty et al., 2007; Lu et al., 2007; Schwarze et al., 2005; St Croix et al., 2000; van Beijnum et al., 2009), and in the pathogenic blood vessels in diabetic retinopathy (Yamaji et al., 2008). This highly specific expression in pathogenic blood vessels is especially well documented for TEM7 (=Tumor Endothelial Marker 7), which was first described in 2000 (St Croix et al., 2000). This high enrichment is not present in healthy blood vessels (Beaty et al., 2007; Lu et al., 2007; Schwarze et al., 2005; St Croix et al., 2000; van Beijnum et al., 2009). We also identified high PLXDC1 expression in choroidal neovascularization (pathogenic angiogenesis in AMD) and ischemia-induced retinopathy (pathogenic angiogenesis in retinopathy of prematurity) (FIG. 1). The striking enrichment of PLXDC1 in the pathogenic blood vessels in several diseases is summarized in the table below:

| | Highly specific | |
|---|---|---|
| | expression in | |
| Pathogenic | pathogenic blood | |
| angiogenesis | vessles | References |
| Tumor blood vessles | Yes | St Croix et al., 2000 |
| in diverse types of | | Carson-Walter et al., 2001 |
| cancer | | Baley et al., 2011 |
| Diabetic retinopathy | Yes | Yamji et al., 2008 |
| Choroidal neovascular- | Yes | FIG. 1 |
| ization | | |
| Ischemia-induced | Yes | FIG. 1 |
| retinopathy | | |

Although PLXDC1/PLXDC2 were known markers of pathogenic blood vessels, it was not known how to effectively target and kill the existing pathogenic blood vessels. In fact, anti-PLXDC1 antibodies have been developed as a potential anti-angiogenic therapy. In Bagley et al., Microvasc Res. 2011 November; 82(3):253-62, an anti-PLXDC1 antibody was identified that mediated antibody-dependent cellular cytotoxicity (ADCC) and phagocytosis. Such cancer immunotherapy approaches, however, have not yielded positive therapeutic results. Furthermore, Pigment Epithelium Derived Factor (PEDF), a natural ligand for PLXDC1 and PLXDC2 with anti-angiogenic properties, failed to kill existing PLXDC-expressing blood vessels. This example demonstrates that agents that modulate the PLXDC1/PLXDC2 receptor can effectively kill existing blood vessels.

A. Ex Vivo Tumor Angiogenesis Model

An ex vivo tumor angiogenesis model was developed to test the efficacy of anti-cancer candidate agents. The following protocol describes the Ex Vivo Tumor Angiogenesis Model.

### Protocol:

1. The day before the experiment, all necessary tools were sprayed with 70% ethanol and sterilized under UV light overnight, including blade, dissecting and micro-dissecting scissors and biceps. 24-well dishes were placed at 4° C. to pre-chill plates and Matrigel was thawed, 24 hours before tumor dissection.
2. 70% ethanol was sprayed on working bench. Two sterile petri dishes were prepared with 10 ml sterile PBS. The following steps 3 and 4 are necessary for mouse tumor models. For fresh human tumor, steps 3 and 4 are skipped.
3. The tumor-bearing mice were euthanized. 70% ethanol was sprayed on the mouse and the tumor was removed using sterilized dissecting tools (avoid the fur). The tumor was rinsed in petri dish with sterile PBS to remove ethanol and fur. The tumor was transferred to a new petri dish with PBS for dissection. The dish is placed on ice.
4. Pre-chilled sterile pipet tips were used to seed regular Matrigel in 24 well plates on ice. Matrigel (30 μl) was dropped in the middle of each well without touching the edge of the well (avoid introducing bubbles if possible).
5. The tumor was cut in halves using the sterile blade. Healthy tumor tissue that is not necrotic and is within the tumor capsule was identified and isolated. The healthy tumor tissue was cut into small pieces. For instance, a suitable size for the tumor tissue is 0.5 mm (H)×0.5 mm (L)×0.3 mm (D) with a total volume of 0.075 mm³.
6. Each tumor piece was gently transferred and embedded in the Matrigel drop in the 24-well plate. The embedded piece was placed in the bottom and middle of each Matrigel drop. The plate was kept on ice all the time.
7. After seeding the tumor pieces, plates were incubated in a 37° C. cell culture incubator without medium for 10 minutes in order for the Matrigel to solidify. 8. Endothelial Growth Medium (0.5 ml) was added to each well and incubated at 37° C. with 5% CO₂. Treatment was not added until the new endothelial cells was grown out of the tumor or were larger than 3 mm in diameter. This usually takes 4 days for the LL2 Lewis lung cancer model and 7 days for the CT26 colon cancer model. For human tumor models, the growth time is typically 2-3 weeks, depending on the tumor type. Media was changed every 4 days during prolonged culture. Typically, when the tumor tissue grows to 2 mm in diameter, it was good for drug testing. A size of about 3 mm in diameter can make visualization easier.
9. When the assay was ready to be analyzed for cell death (e.g., 48 hours after drug addition), the dye mixture was prepared by mixing 6 μl of green dye to stain live cells (5 mg/ml Fluorescein diacetate or FDA in DMSO) with 30 μl of red dye (2.5 mg/ml Propidium iodide or PI in PBS) to stain dead cells in an Eppendorf tube. The FDA dye needs to be stored frozen in a −20° C. freezer because it has a labile ester bond.
10. 1 μl of the dye mixture was added to each well of the 24-well dish. It is usually preferred to do one 24-well dish at a time given the amount of time needed to take pictures (the green dye is not as stable in the cells in the long term).
11. The dish was gently rocked a few times to mix the dye with the media in the wells and incubate the dish at 37° C. for 10 min (too long incubation can make the green signal too intense).
12. Each well was washed with 0.5 ml of sterile PBS and then 0.5 ml of phenol red free SFM was added to each well. Alternatively 0.5 ml of regular Endothelial Cell Growth Media can be added to each well if this well needs to be continuously maintained after the experiment.
13. An inverted microscope using the 2x objective lens was used to observe morphological changes in the experimental wells.
14. Taking pictures in the red and green channels would allow not only the recording of the results but also more accurate quantitation of the results. To take pictures for all the wells, first a well that has robust red and green signals was picked. A picture at the red channel using the optimal setting (remember this setting) was taken and then a picture at the green channel using another optimal setting (remember this setting) was taken. The final picture is the merged picture of the red and green channels. Pictures of all other wells were taken in each channel using the same settings so that different wells can be compared.

B. Results

Using the model described above, the inventors designed and tested novel compounds and screened monoclonal antibodies of PLXDC1 for their ability to kill pathogenic blood vessels.

1. Killing pathogenic blood vessels in vision diseases

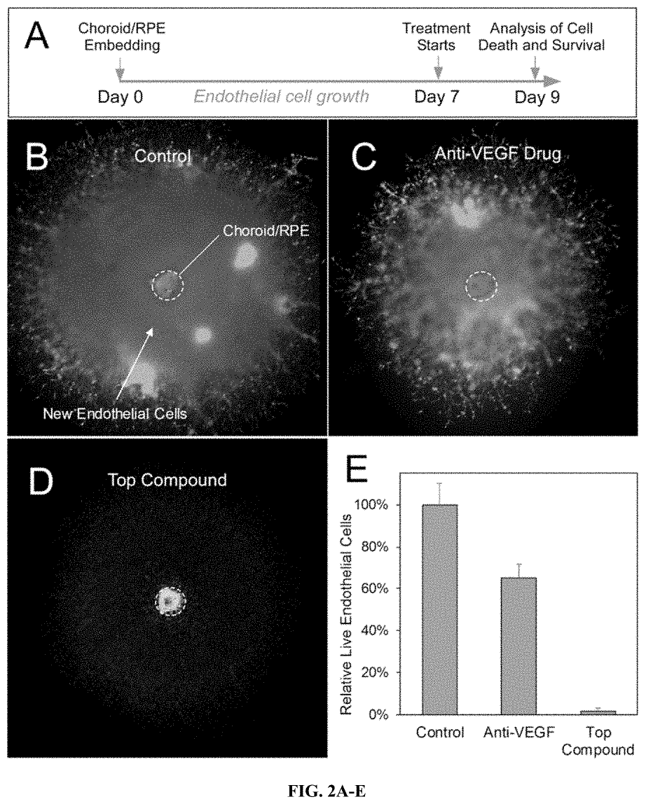

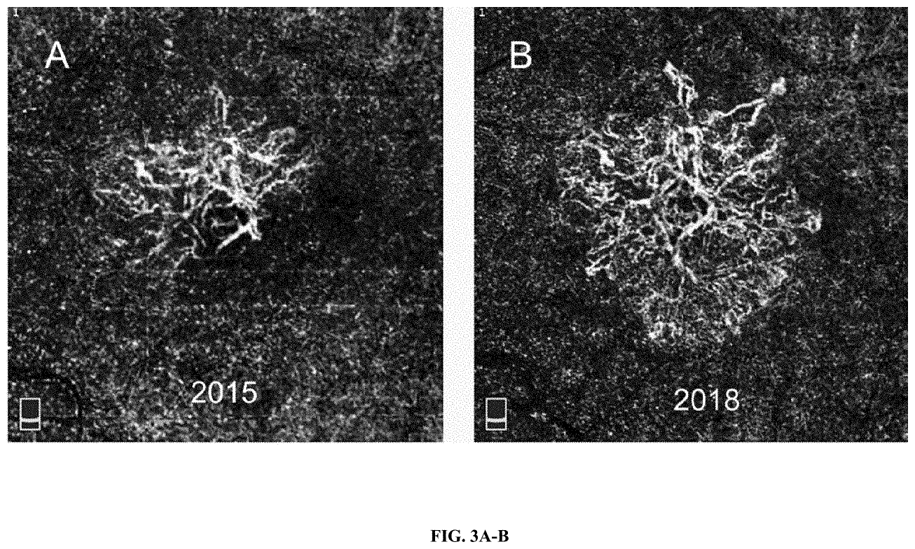

FIG. 2 demonstrates that compound 369 (Table 3) targeting PLXDC1/PLXDC2 can kill the endothelial cells in an ex vivo model of choroidal neovascularization (see, for example, Shao, Z. et al., PLoS One. 2013 Jul. 26; 8(7):e69552, which is herein incorporated by reference) without affecting the healthy tissue (choroid and retinal pigment epithelium (RPE)). In contrast and as demonstrated in FIG. 3, the current anti-angiogenic drug Eylea (currently the most commonly used drug for this purpose) can only inhibit angiogenesis, but does not kill existing endothelial cells from choroidal angiogenesis (even at a concentration much higher than what is used clinically). FIG. 3 is an example of human data showing that 24 injections of anti-VEGF drug Eylea over three years only partially inhibits pathogenic blood vessel growth but cannot kill pathogenic blood vessels.

2. Killing tumor blood vessels

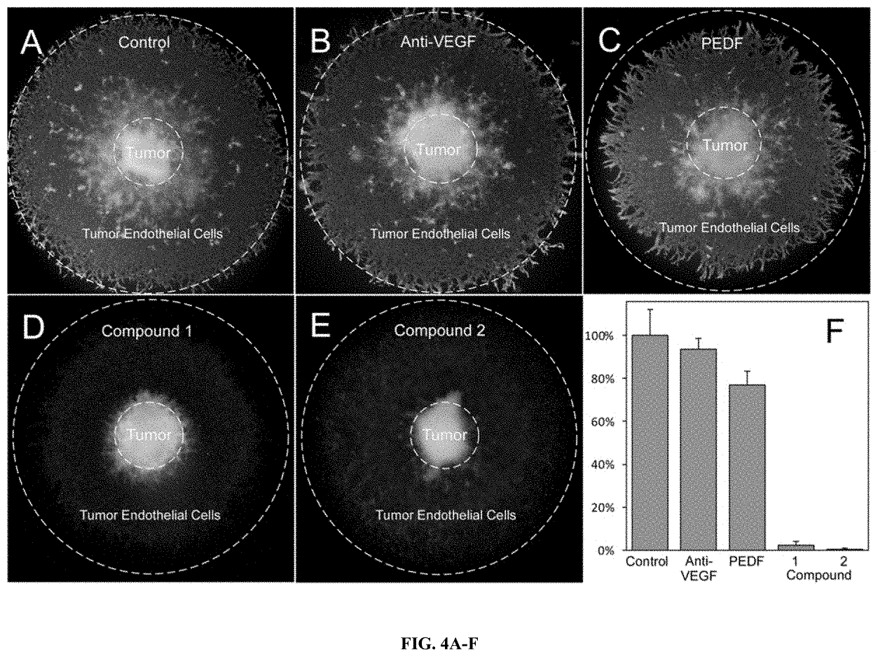

Previously there has been no drug (small molecule or large molecule) that can kill tumor (causing massive tumor necrosis) by killing existing tumor blood vessels. The reason why existing drugs that target blood vessels cannot achieve this therapeutic effect is that they are anti-angiogenic drugs and are designed to inhibit new blood vessel growth (and tumor growth). FIG. 4 shows that top compounds (e.g., compound 369) of the present disclosure targeting PLXDC1/PLXDC2 can kill existing tumor endothelial cells in an ex vivo model of tumor angiogenesis. In contrast, the current anti-angiogenic drug or anti-angiogenic factor PEDF cannot kill existing tumor endothelial cells.

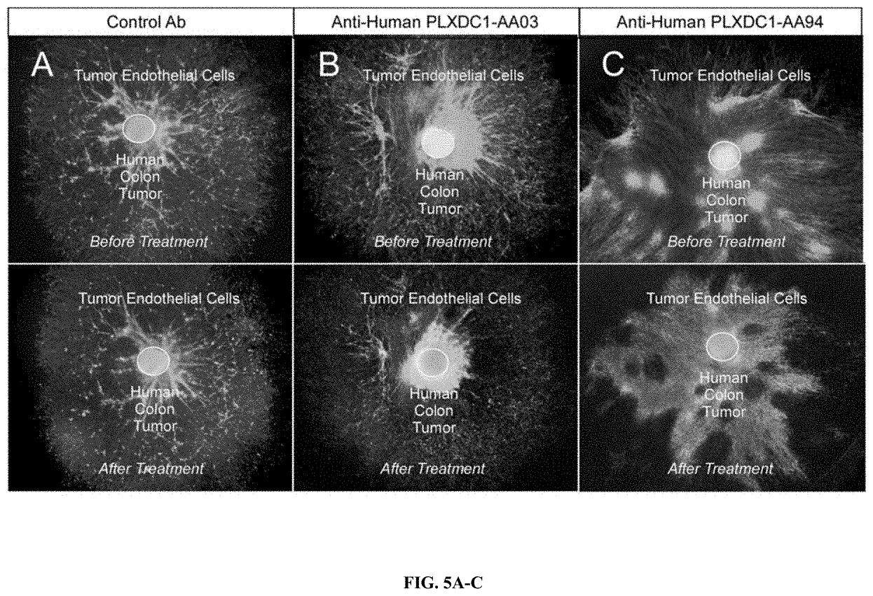

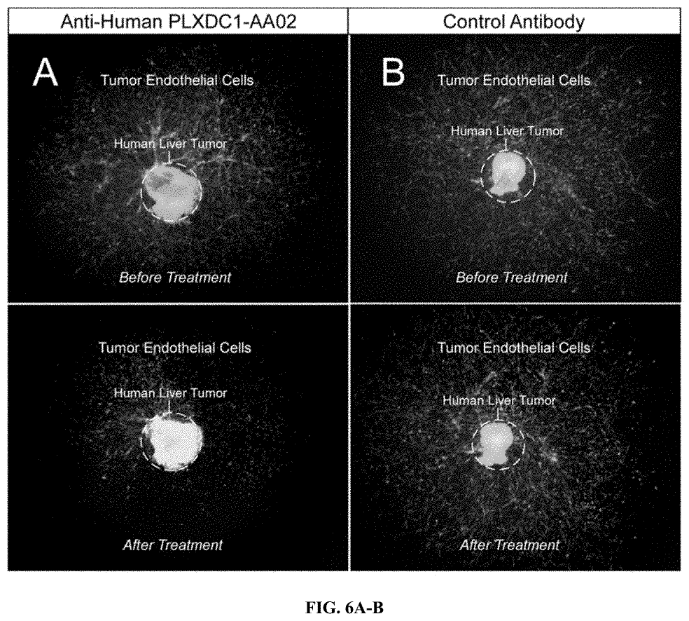

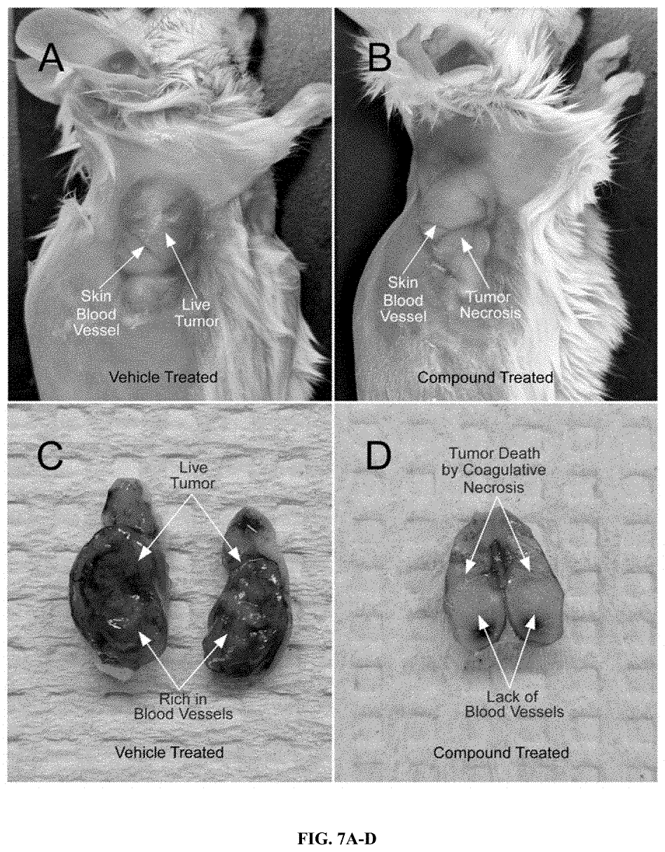

FIGS. 5 and 6 show that antibodies of the disclosure that target human PLXDC1 can kill existing tumor endothelial cells in ex vivo models of human tumor angiogenesis. FIG. 7 is an in vivo experiment demonstrating that systemic administration of compound 369 targeting PLXDC1/PLXDC2 can kill existing tumor blood vessels and lead to strong tumor death. The tumor died of massive coagulative necrosis, exactly the kind of cell death caused by killing blood vessels.

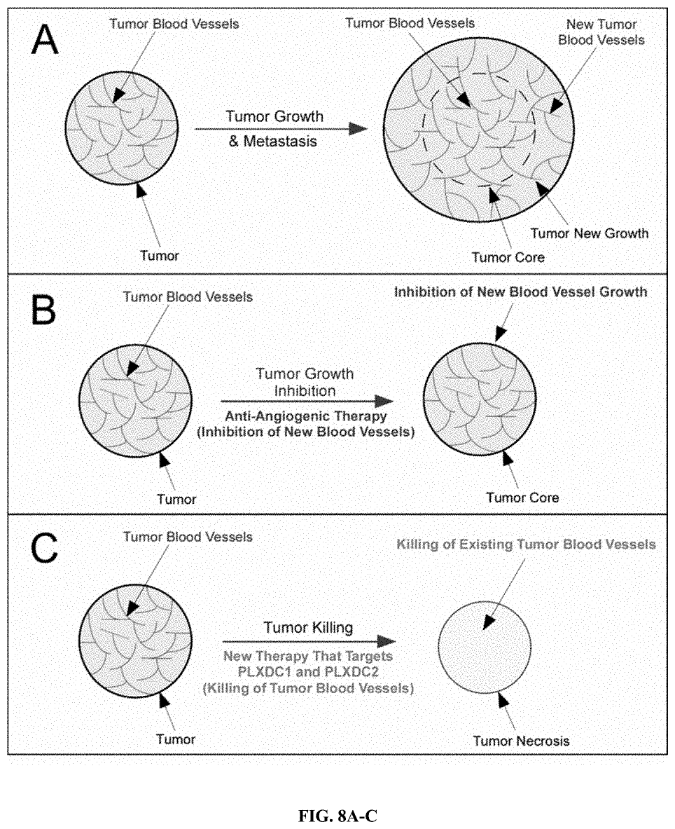

FIG. 8 illustrates the differences between this new mechanism and anti-angiogenic drugs. Angiogenesis is the growth of new blood vessels from existing blood vessels. Tumor depends on angiogenesis to grow and to metastasize (FIG. 8A). Antiangiogenic drugs or factors inhibit angiogenesis and the growth of the tumor, but cannot kill the tumor because they cannot effectively kill existing tumor blood vessels (FIG. 8B). Drugs that target PLXDC1/PLXDC2 can kill existing tumor blood vessels to cause tumor death (FIG. 8C). Accordingly, agents and methods of the present disclosure are able to kill existing blood vessels associated with the tumors and thus are more potent in tumor treatment.

3. Binding Agent/Receptor Interaction

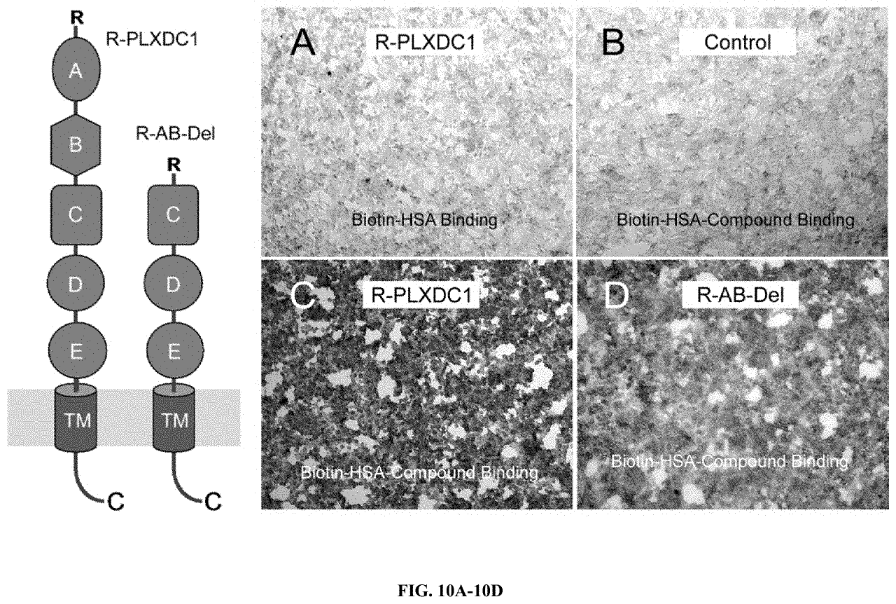

It was found that certain small molecule drug candidates and anti-PLXDC1 monoclonal antibodies can target PLXDC1 to kill pathogenic blood vessels. Further, these molecules can interact with PLXDC1 independently of Domain B, which is required for PEDF binding (FIG. 10).

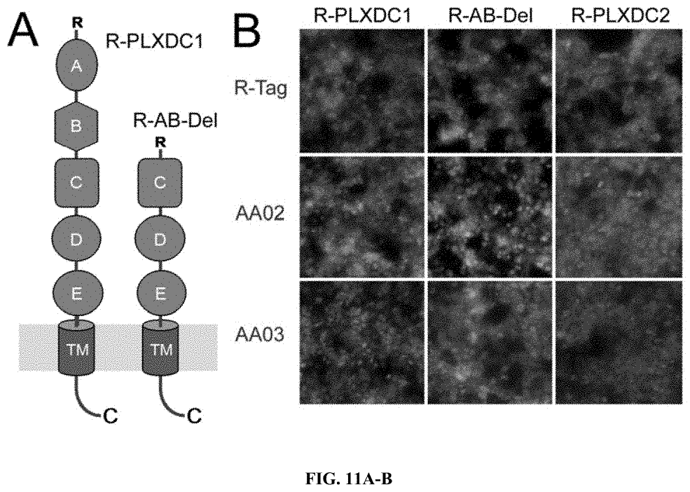

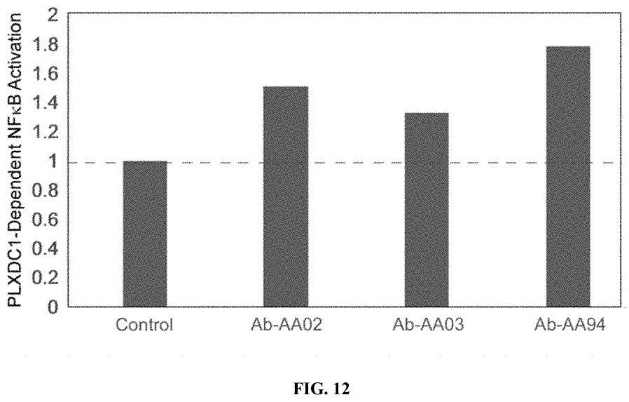

Similarly, it was found that antibody clones such as AA02, AA03, and AA94 interacted independently of domain B (FIG. 11), activated NF-κB (FIG. 12) and also killed tumor endothelial cells (FIG. 5B-C, FIG. 6A). It is not surprising that PLXDC1, which has a large extracellular domain, has multiple sites for binding to factors and ligands.

Many receptors are known to bind to different ligands. PEDF is known to bind to PLXDC1. Like all anti-angiogenic factors, PEDF can suppress angiogenesis, the growth of new blood vessels, but cannot kill existing blood vessels. In contrast, the compounds of the disclosure that target PLXDC1/PLXDC2 can kill tumor blood vessels.

FIG. 4 shows a direct comparison between compounds of the disclosure, PEDF, and an anti-angiogenic agent, VEGF trap. In contrast to the PLXDC1/PLXDC2 modulators of the disclosure, PEDF, which interacts with Domain B of PLXDC1, didn't kill pathogenic blood vessels. In summary, it was found that antibodies and compounds of the disclosure were able to kill pathogenic blood vessels while PEDF, a natural ligand for PLXDC1/PLXDC2 and some current anti-angiogenic therapies, were not able to achieve this effect. This data exemplifies the importance of this novel therapeutic strategy for killing blood vessels and differentiates this strategy from current anti-angiogenic strategies that halt the development of new blood vessels.

### Example 2. Testing of Small Molecules in Mouse Model of Colon Cancer

This example shows that the Ex Vivo Tumor Angiogenesis Model described in Example 1 is effective in screening for agents capable of killing tumor blood vessels.

Select compounds from the following table were tested in the following examples.

A tumor from xenograft mouse model of colon cancer (CT26.CL25) was grown using the method described in Example 1 to establish an ex vivo model of tumor angiogenesis. Treatment did not start until the new tumor endothelial cells had grown for 7 days. After drug treatment was done for two days, cell survival was assessed by a two-color assay using a mixture of fluorescein diacetate (green dye) and propidium iodide (red dye). Green cells represented live cells. Red cells represented dead cells. Orange cells represented a mixture of live and dead cells.

The percent cell death is calculated according the ratio of the red area and total endothelial cell area.

The activity of the tested compounds is provided in Table 4 below. Morphology observations after 48 hours are provided under the column “48 hrs,” wherein: “0” indicates all cells have normal endothelial cell morphology (cells are elongated and connect to neighbor cells); “*” indicates 50% or less of cells vesicularize in cell shape; “**” indicates more than 50% but less than 100% of endothelial cells vesicularize in cell shape; “***” indicates 100% of endothelial cells vesicularize in cell shape; “****” indicates 100% of endothelial cells vesicularize in cell shape and look flattened in morphology (indicating disintegration of the cell body). Vesicularization in cell shape indicates that the endothelial cells no longer have the elongated shape and no longer connect to neighbor cells.

TABLE 4

| Compound | Colon CT26 ex vivo model of tumor angiogenesis | | |
|---|---|---|---|
| No. | 48 hrs | Cell death | Conc. (μm) |
| 42 | 0 | 0% | 40 |
| 43 | *** | 90% | 40 |
| 44 | 0 | 0% | 40 |
| 45 | 0 | 0% | 40 |
| 46 | **** | 100% | 40 |
| 47 | 0 | 10% | 40 |
| 48 | 0 | 10% | 40 |
| 49 | 0 | 10% | 40 |
| 50 | *** | 100% | 40 |
| 51 | *** | 20% | 40 |
| 52 | 0 | 0% | 40 |
| 54 | 0 | 10% | 40 |
| 55 | 0 | 50% | 40 |
| 56 | 0 | 30% | 40 |
| 57 | * | 60% | 40 |
| 58 | * | 50% | 40 |
| 59 | 0 | 50% | 40 |
| 60 | 0 | 5% | 40 |
| 61 | 0 | 20% | 40 |
| 62 | ** | 70% | 40 |
| 63 | *** | 70% | 40 |
| 64 | *** | 100% | 40 |
| 65 | 0 | 0% | 40 |
| 66 | 0 | 0% | 40 |
| 67 | 0 | 0% | 40 |
| 68 | ** | 50% | 40 |
| 69 | 0 | 0% | 40 |
| 70 | 0 | 10% | 40 |
| 71 | 0 | 0% | 40 |
| 72 | 0 | 0% | 40 |
| 73 | 0 | 0% | 40 |
| 74 | *** | 100% | 40 |
| 75 | 0 | 0% | 40 |
| 76 | ** | 100% | 40 |
| 77 | 0 | 40% | 40 |
| 78 | 0 | 0% | 40 |
| 79 | *** | 100% | 10 |
| 80 | *** | 100% | 10 |
| 81 | 0 | 0% | 40 |
| 82 | 0 | 10% | 40 |
| 84 | * | 100% | 40 |
| 85 | 0 | 50% | 40 |
| 86 | *** | 100% | 10 |
| 87 | 0 | 0% | 40 |
| 88 | *** | 60% | 40 |
| 89 | *** | 100% | 10 |
| 90 | 0 | 0% | 40 |
| 91 | *** | 100% | 10 |
| 92 | 0 | 0% | 40 |
| 93 | *** | 100% | 10 |
| 94 | 0 | 0% | 40 |
| 95 | 0 | 0% | 40 |
| 96 | 0 | 0% | 10 |
| 97 | *** | 100% | 10 |
| 98 | *** | 50% | 10 |
| 99 | 0 | 0% | 40 |
| 100 | **** | 100% | 40 |
| 101 | * | 50% | 20 |
| 102 | 0 | 5% | 40 |
| 110 | ** | 10% | 40 |
| 112 | ** | 10% | 40 |
| 116 | * | 50% | 20 |
| 117 | *** | 100% | 20 |
| 118 | *** | 100% | 20 |
| 119 | 0 | 50% | 20 |
| 121 | *** | 40% | 40 |
| 123 | 0 | 20% | 40 |
| 124 | 0 | 20% | 40 |
| 128 | 0 | 10% | 40 |
| 129 | 0 | 0% | 40 |
| 130 | 0 | 40% | 40 |
| 131 | ** | 50% | 40 |
| 132 | * | 40% | 40 |
| 135 | 0 | 30% | 40 |
| 136 | 0 | 30% | 40 |
| 137 | 0 | 40% | 40 |
| 138 | 0 | 20% | 40 |
| 139 | 0 | 40% | 40 |
| 140 | ** | 60% | 40 |
| 144 | 0 | 30% | 40 |
| 145 | 0 | 30% | 40 |
| 146 | 0 | 0% | 40 |
| 147 | 0 | 0% | 40 |
| 148 | 0 | 0% | 40 |
| 149 | * | 20% | 40 |
| 150 | 0 | 10% | 40 |
| 151 | **** | 100% | 40 |
| 152 | ** | 30% | 40 |
| 153 | * | 20% | 40 |
| 154 | * | 30% | 40 |
| 155 | *** | 20% | 40 |
| 156 | 0 | 0% | 40 |
| 157 | **** | 60% | 40 |
| 158 | **** | 100% | 40 |
| 159 | **** | 100% | 40 |
| 160 | **** | 100% | 40 |
| 161 | **** | 100% | 40 |
| 162 | *** | | 40 |
| 163 | **** | 100% | 40 |
| 164 | **** | 100% | 40 |
| 165 | 0 | 10% | 40 |
| 166 | *** | 60% | 40 |
| 191 | 0 | 10% | 40 |
| 192 | 0 | 10% | 40 |
| 201 | * | 10% | 40 |
| 202 | 0 | 10% | 40 |
| 206 | **** | 100% | 40 |
| 207 | **** | 100% | 40 |
| 208 | 0 | 20% | 40 |
| 209 | *** | 90% | 40 |
| 210 | *** | 70% | 40 |
| 211 | **** | 100% | 40 |
| 212 | 0 | 10% | 40 |
| 214 | **** | 100% | 40 |
| 215 | 0 | 20% | 40 |
| 216 | **** | 100% | 40 |
| 218 | *** | 90% | 40 |
| 219 | *** | 90% | 40 |
| 220 | **** | 100% | 40 |
| 221 | **** | 100% | 40 |
| 238 | 0 | 10% | 10 |
| 241 | 0 | 0% | 10 |
| 253 | *** | 80% | 10 |
| 254 | ** | 40% | 10 |
| 255 | 0 | 0% | 10 |
| 256 | *** | 90% | 10 |
| 257 | **** | 100% | 10 |
| 258 | *** | 100% | 10 |
| 259 | *** | 100% | 20 |

### Example 3. Testing of Compounds in Mouse Lung Cancer Model

This example tested the compounds of Example 2 in a mouse lung cancer model.

A tumor from xenograft mouse model of lung cancer (LL/2) was grown using the methods described in Example 1 to establish an ex vivo model of tumor angiogenesis. Treatment did not start until the new tumor endothelial cells had grown for 5 days. After drug treatment was done for two days, cell survival was assessed by a two-color assay using a mixture of fluorescein diacetate (green dye) and propidium iodide (red dye). Green cells represented live cells. Red cells represented dead cells. Orange cells represented a mixture of live and dead cells. The percent cell death is calculated according the ratio of the red area and total endothelial cell area.

The activity of the tested compounds is provided in Table 5 below. Morphology observations after 48 hours are provided under the column “48 hrs,” wherein: “0” indicates all cells have normal endothelial cell morphology (cells are elongated and connect to neighbor cells); “*” indicates 50% or less of cells vesicularize in cell shape; “**” indicates more than 50% but less than 100% of endothelial cells vesicularize in cell shape; “***” indicates 100% of endothelial cells vesicularize in cell shape; “****” indicates 100% of endothelial cells vesicularize in cell shape and look flattened in morphology (indicating disintegration of the cell body). Vesicularization in cell shape indicates that the endothelial cells no longer have the elongated shape and no longer connect to neighbor cells.

TABLE 5

| | Lung LL2 ex vivo model of tumor angiogenesis | | |
|---|---|---|---|
| No | 48 hrs | Cell death | Conc. (μm) |
| 42 | 0 | 30% | 40 |
| 43 | *** | 100% | 40 |
| 44 | ** | 50% | 40 |
| 45 | ** | 10% | 40 |
| 46 | **** | 100% | 40 |
| 47 | 0 | 50% | 40 |
| 48 | ** | 60% | 40 |
| 49 | 0 | 30% | 40 |
| 50 | *** | 90% | 40 |
| 51 | **** | 100% | 40 |
| 52 | ** | 50% | 40 |
| 54 | 0 | 50% | 40 |
| 55 | ** | 70% | 40 |
| 56 | * | 20% | 40 |
| 57 | 0 | 40% | 40 |
| 58 | 0 | 50% | 40 |
| 59 | 0 | 40% | 40 |
| 61 | 0 | 50% | 40 |
| 62 | *** | 30% | 40 |
| 63 | ** | 50% | 40 |
| 64 | *** | 100% | 40 |
| 65 | 0 | 10% | 40 |
| 66 | 0 | 10% | 40 |
| 67 | 0 | 10% | 40 |
| 68 | *** | 80% | 40 |
| 69 | 0 | 20% | 40 |
| 70 | *** | 40% | 40 |
| 71 | * | 10% | 40 |
| 72 | 0 | 20% | 40 |
| 73 | 0 | 10% | 40 |
| 74 | *** | 60% | 40 |
| 75 | ** | 80% | 40 |
| 76 | 0 | 20% | 40 |
| 77 | ** | 80% | 40 |
| 78 | 0 | 40% | 40 |
| 79 | ** | 20% | 20 |
| 80 | *** | 100% | 20 |
| 81 | 0 | 0% | 40 |
| 82 | 0 | 60% | 40 |
| 83 | 0 | 20% | 40 |
| 84 | ** | 100% | 40 |
| 85 | 0 | 80% | 40 |
| 86 | *** | 100% | 20 |
| 87 | 0 | 0% | 40 |
| 88 | 0 | 70% | 40 |
| 89 | *** | 100% | 20 |
| 90 | 0 | 10% | 40 |
| 91 | *** | 80% | 20 |
| 92 | 0 | 10% | 20 |
| 93 | *** | 100% | 20 |
| 94 | 0 | 20% | 40 |
| 95 | 0 | 10% | 40 |
| 96 | 0 | 0% | 20 |
| 97 | ** | 50% | 20 |
| 98 | ** | 10% | 20 |
| 99 | 0 | 10% | 40 |
| 100 | **** | 100% | 40 |
| 101 | *** | 100% | 20 |
| 102 | 0 | 20% | 20 |
| 103 | 0 | 10% | 20 |
| 104 | 0 | 20% | 20 |
| 105 | 0 | 0% | 20 |
| 106 | 0 | 5% | 20 |
| 107 | 0 | 0% | 20 |
| 108 | 0 | 0% | 20 |
| 109 | 0 | 0% | 20 |
| 110 | *** | 95% | 20 |
| 111 | 0 | 30% | 20 |
| 112 | *** | 70% | 20 |
| 113 | 0 | 10% | 20 |
| 114 | 0 | 5% | 20 |
| 115 | 0 | 10% | 20 |
| 116 | *** | 100% | 20 |
| 117 | *** | 100% | 20 |
| 118 | *** | 100% | 20 |
| 119 | *** | 100% | 20 |
| 120 | 0 | 5% | 20 |
| 121 | ** | 50% | 40 |
| 122 | 0 | 10% | 20 |
| 125 | * | 10% | 20 |
| 126 | 0 | 10% | 20 |
| 133 | ** | 10% | 20 |
| 134 | **** | | 20 |
| 141 | **** | 90% | 20 |
| 142 | **** | | 20 |
| 143 | 0 | 40% | 40 |
| 151 | **** | 100% | 40 |
| 152 | * | 20% | 20 |
| 154 | * | 30% | 40 |
| 155 | *** | 80% | 40 |
| 156 | ** | 30% | 40 |
| 157 | *** | 100% | 40 |
| 158 | **** | 100% | 40 |
| 159 | **** | 100% | 40 |
| 160 | **** | 100% | 40 |
| 161 | **** | 90% | 40 |
| 162 | *** | 70% | 40 |
| 163 | **** | 95% | 40 |
| 164 | **** | 95% | 40 |
| 165 | * | 10% | 40 |
| 166 | 0 | 10% | 40 |
| 167 | 0 | 40% | 20 |
| 168 | 0 | 10% | 20 |
| 169 | 0 | 40% | 20 |
| 170 | 0 | 10% | 20 |
| 171 | 0 | 40% | 40 |
| 172 | **** | 100% | 40 |
| 173 | ** | 50% | 40 |
| 174 | 0 | 30% | 40 |
| 175 | 0 | 30% | 40 |
| 176 | *** | 100% | 40 |
| 177 | **** | 100% | 40 |
| 178 | **** | 100% | 40 |
| 179 | *** | 60% | 10 |
| 180 | *** | 70% | 10 |
| 181 | **** | 100% | 40 |
| 182 | **** | 100% | 40 |
| 183 | **** | 100% | 10 |
| 184 | 0 | 5% | 10 |
| 185 | * | 30% | 10 |
| 186 | **** | 90% | 10 |
| 187 | 0 | 30% | 40 |
| 191 | 0 | 30% | 40 |
| 192 | *** | 60% | 40 |
| 193 | 0 | 30% | 40 |
| 195 | 0 | | 40 |
| 196 | 0 | 30% | 40 |
| 197 | 0 | 20% | 40 |
| 198 | 0 | 30% | 40 |
| 199 | 0 | 30% | 40 |
| 200 | 0 | 30% | 40 |
| 201 | 0 | 30% | 40 |
| 202 | ** | 30% | 40 |
| 206 | 0 | 30% | 20 |
| 207 | ** | | 20 |
| 208 | 0 | 50% | 40 |
| 209 | * | 60% | 40 |
| 210 | 0 | 50% | 40 |
| 211 | **** | 100% | 40 |
| 212 | 0 | 50% | 40 |
| 213 | 0 | 30% | 40 |
| 214 | **** | 100% | 40 |
| 215 | 0 | 30% | 40 |
| 216 | **** | 100% | 40 |
| 217 | 0 | 30% | 40 |
| 218 | **** | 100% | 40 |
| 219 | **** | 60% | 40 |
| 220 | **** | 100% | 40 |
| 221 | **** | 100% | 40 |
| 223 | *** | 30% (rim) | 40 |
| 225 | 0 | 40% | 40 |
| 226 | * | 10% | 40 |
| 227 | 0 | 10% | 40 |
| 228 | 0 | 5% | 40 |
| 229 | 0 | 30% | 40 |
| 230 | *** | 100% | 40 |
| 231 | *** | 100% | 40 |
| 232 | 0 | 10% | 40 |
| 233 | 0 | 5% | 40 |
| 234 | *** | 100% | 40 |
| 235 | 0 | 10% | 40 |
| 236 | 0 | 5% | 40 |
| 237 | *** | 100% | 40 |
| 238 | *** | 100% | 40 |
| 239 | 0 | 40% | 40 |
| 240 | 0 | 30% | 40 |
| 241 | 0 | 40% | 40 |
| 242 | 0 | 30% | 40 |
| 243 | 0 | 20% | 40 |
| 244 | 0 | 20% | 40 |
| 245 | 0 | 40% | 40 |
| 246 | *** | 95% | 20 |
| 247 | *** | 60% | 20 |
| 248 | * | 60% | 20 |
| 249 | *** | 60% | 20 |
| 250 | *** | 90% | 20 |
| 251 | *** | 60% | 20 |
| 253 | **** | 100% | 20 |
| 254 | *** | 100% | 20 |
| 255 | * | 10% | 20 |
| 256 | **** | 100% | 20 |
| 257 | **** | 100% | 20 |
| 258 | **** | 100% | 20 |
| 259 | *** | 95% | 20 |
| 262 | 0 | 20% | 10 |
| 263 | **** | 100% | 10 |
| 264 | * | 20% | 10 |
| 265 | * | 20% | 10 |
| 266 | 0 | 5% | 10 |
| 267 | 0 | 5% | 10 |
| 268 | *** | 50% | 20 |
| 269 | **** | 100% | 20 |
| 270 | * | 50% | 20 |
| 271 | *** | 60% | 20 |
| 272 | * | 20% | 20 |
| 273 | *** | 60% | 20 |
| 274 | 0 | 10% | 20 |
| 275 | 0 | 5% | 20 |
| 276 | *** | 100% | 20 |
| 277 | ** | 60% | 10 |
| 278 | * | 50% | 10 |
| 279 | **** | 100% | 20 |
| 280 | *** | 100% | 20 |
| 281 | ** | 50% | 20 |
| 282 | 0 | 10% | 10 |
| 283 | 0 | 5% | 10 |
| 284 | 0 | 10% | 10 |
| 285 | *** | 60% | 10 |
| 286 | 0 | 0% | 20 |
| | (24 hrs) | (24 hrs) | |
| 287 | * | 40% | 20 |
| | (24 hrs) | (24 hrs) | |
| 288 | *** | 50% | 10 |
| 289 | ** | 20% | 20 |
| 290 | *** | 50% | 10 |
| 291 | * | 10% | 20 |
| | (24 hrs) | (24 hrs) | |
| 292 | * | 10% | 20 |
| | (24 hrs) | (24 hrs) | |
| 294 | * | 50% | 40 |
| 296 | 0 | 20% | 40 |
| 300 | 0 | 20% | 40 |
| 302 | 0 | 70% | 40 |
| 306 | 0 | 40% | 40 |
| 308 | 0 | 20% | 40 |
| 310 | 0 | 10% | 40 |
| 311 | 0 | 30% | 40 |
| 312 | **** | 100% | 40 |
| 317 | 0 | 30% | 40 |
| 318 | 0 | 30% | 40 |
| 320 | 0 | 30% | 40 |
| 321 | 0 | 40% | 40 |
| 322 | 0 | 30% | 40 |
| 323 | 0 | 30% | 40 |
| 324 | 0 | 60% | 40 |
| 325 | * | 30% | 40 |
| 326 | 0 | 30% | 40 |
| 327 | 0 | 20% | 40 |
| 328 | 0 | 30% | 40 |
| 329 | *** | 40% | 40 |
| 330 | *** | 50% | 40 |
| 331 | 0 | 10% | 40 |
| 332 | 0 | 0% | 40 |
| 333 | 0 | 0% | 40 |
| 336 | 0 | 0% | 40 |
| 339 | ** | 50% | 20 |
| 340 | *** | 60% | 20 |
| 341 | *** | 80% | 20 |
| 342 | *** | 60% | 20 |
| 343 | *** | 50% | 20 |
| 344 | * | 10% | 20 |
| 345 | *** | 50% | 20 |
| 346 | * | 20% | 20 |
| 347 | *** | 30% | 20 |
| 348 | * | 20% | 20 |
| 350 | 0 | 20% | 40 |
| 351 | *** | 100% | 40 |
| 352 | *** | 100% | 40 |
| 353 | 0 | 0% | 40 |
| 354 | 0 | 0% | 40 |
| 355 | 0 | 0% | 40 |
| 356 | 0 | 0% | 40 |
| 357 | 0 | 0% | 40 |
| 358 | 0 | 0% | 40 |
| 359 | 0 | 20% | 40 |
| 360 | 0 | 20% | 40 |
| 362 | *** | 20% | 20 |
| 363 | *** | 30% | 20 |
| 364 | *** | 30% | 20 |
| 365 | *** | 20% | 20 |
| 366 | 0 | 10% | 20 |
| 367 | 0 | 5% | 20 |
| 368 | ** | 20% | 20 |
| 369 | * | 20% | 20 |
| 370 | ** | 20% | 20 |
| 371 | *** | 30% | 20 |
| 372 | ** | 20% | 20 |
| 373 | * | 20% | 20 |
| 374 | ** | 20% | 20 |
| 375 | * | 50% | 20 |
| 376 | 0 | 10% | 20 |
| 377 | 0 | 10% | 20 |
| 378 | *** | 30% | 20 |

Certain other compounds described herein also showed activity in causing endothelial cell death in other angiogenesis assays.

### Example 4. Generation of PLXDC-Activating Antibodies

This example describes the generation of monoclonal antibodies that bind and activate the PLXDC proteins.

To the instant inventors' knowledge, no antibody drug exists that can active a cell-surface receptor. All existing antibody drugs are neutralizing antibodies that inhibit the ligand/receptor interaction, such as Humira (inhibiting TNF-α, a ligand), Avastin (inhibiting VEGF, a ligand), Herceptin (inhibiting HER2, a receptor), and Keytruda (inhibiting PD-1, a receptor). It was, therefore, unexpected that the antibodies tested in Example 1 were able to activate PLXDC1, a single transmembrane cell-surface receptor. This example designed and tested a procedure that used it to obtain a large number of highly potent activating antibodies.

At a first step, a customized human antibody library was created, which contained about 10 billion antibody clones. To screen for activating antibodies from this library, an screening assay was established with the human PLXDC1 protein (biotinylated at a biotin/PLXDC1 ratio of about 2.7) in the presence of an activating small molecule compound (e.g., compound 369 of Table 3).

About 100 human antibody clones were identified with this method that preferentially bound to the small molecule-activated PLXDC1. The VH/VL sequences, and the corresponding CDR sequences of selected ones are presented in the tables below.

TABLE 6
Select PLXDC1-Activating Antibodies

| | | SEQ ID |
|---|---|---|
| Name | Sequence | NO: |
| 1-A1_VH | EVQLLESGGGLVQPGGSLRLSCAASGFTFTSYAMSWVRQAPGKGLEWVSAISAG | 4 |
| | GGYTYYADSVKGRFTISRDNSKNTLYLQMNSLRAEDTAVYYCARYDYNSYFDSW | |
| | GQGTLVTVSS | |
| 1-A1_VL | QSTQPPSVSGAPGQRVTISCTGSSSNIGAGYDVHWYQQLPGTAPKLLIYGNNNR | 5 |
| | PSGVPDRFSGSKSGTSASLAITGLQAEDEADYYCQSYDRSLSGWVFGGGTKLTV | |
| | L | |
| 1-A5_VH | QVQLVESGGGVVQPGRSLRLSCAASGYAFSSYGMHWVRQAPGKGLEWVAVISHS | 6 |
| | GSNKYYANSVKGRFTISRDNSKNTLYLQMNSLRAEDTAVYYCARGLAGWEVYPF | |
| | DVWGQGTLVTVSS | |
| 1-A5_VL | QSTQPPSVSGAPGQRVTISCTGSSSNIGAGEDVHWYQQLPGTAPKLLIYDNTNR | 7 |
| | PSGVPDRFSGSKSGTSASLAITGLQAEDEADYYCQSYDYSLRVWVFGGGTKLTV | |
| | L | |
| 1- | EVQLLESGGGLVQPGGSLRLSCAASGFTFSNYAMSWVRQAPGKGLEWVSTISGG | 8 |
| H10_VH | GTYYADSVKGRFTISRDNSKNTLYLQMNSLRAEDTAVYYCARSRYWYTKLISYT | |
| | YGMDVWGQGTLVTVSS | |
| 1- | QSVLTQPPSVSGAPGQRVTISCTGSSSNIGATYDVHWYQQLPGTAPKLLIYVNN | 9 |
| H10_VL | NRPSGVPDRFSGSKSGTSASLAITGLQAEDEADYYCQSYDSSLRAWVFGGGTKL | |
| | TVL | |
| 2-B4_VH | QVQLVESGGGVVQPGRSLRLSCAASGFTFSNYGMHWVRQAPGKGLEWVAVISYH | 10 |
| | GRNEYYADSVKGRFTISRDNSKNTLYLQMNSLRAEDTAVYYCARGDDQDAFDPW | |
| | GQGTLVTVSS | |
| 2-B4_VL | DIQMTQSPSSLSASVGDRVTITCRASQSISNYLNWYQQKPGKAPKLLIYAASSL | 11 |
| | QSGVPSRFSGSGSGTDFTLTISSLQPEDFATYYCQQSFSAPYTFGQGTKVEIK | |
| 2-B5_VH | QVQLVESGGGVVQPGRSLRLSCAASGFIFSDYDMHWVRQAPGKGLEWVAVISHS | 12 |
| | GSNKYYADSVKGRFTISRDNSKNTLYLQMNSLRAEDTAVYYCARDRSVYLYYGY | |
| | HYYEGFDVWGQGTLVTVSS | |
| 2-B5_VL | EIVMTQSPATLSVSPGERATLSCRASQSVYNNLAWYQQKPGQAPRLLIYGASTR | 13 |
| | ATGIPARFSGSGSGTEFTLTISSLQSEDFAVYYCQQYNDLPLTFGQGTKVEIK | |
| 2-F7_VH | EVQLLESGGGLVQPGGSLRLSCAASGFTFSDYAMSWVRQAPGKGLEWVSSIYTS | 14 |
| | GGRTYYADSVKGRFTISRDNSKNTLYLQMNSLRAEDTAVYYCARGYRYFDHWGQ | |
| | GTLVTVSS | |
| 2-F7_VL | EIVMTQSPATLSVSPGERATLSCRASQSVRNNLAWYQQKPGQAPRLLIYGASTR | 15 |
| | ATGIPARFSGSGSGTEFTLTISSLQSEDFAVYYCQQYYTWPRTFGQGTKVEIK | |
| 2-F8_VH | EVQLLESGGGLVQPGGSLRLSCAASGFTFPNYAMSWVRQAPGKGLEWVSTIYGR | 16 |
| | GERTYYADSVKGRFTISRDNSKNTLYLQMNSLRAEDTAVYYCARVSVVWSYAFD | |
| | YWGQGTLVTVSS | |
| 2-F8_VL | DIQMTQSPSSLSASVGDRVTITCRASQDISTYLNWYQQKPGKAPKLLIYAASSL | 17 |
| | QSGVPSRFSGSGSGTDFTLTISSLQPEDFATYYCQQSYSYPLTFGQGTKVEIK | |
| 2-G4_VH | QLQLQESGPGLVKPSETLSLTCTVSGGSISSSNYYWGWIRQPPGKGLEWIGSVY | 18 |
| | YTGRTYYNPSLKSRVTISVDTSKNQFSLRLSSVTAADTAVYYCARVFPYGAVDV | |
| | WGQGTLVTVSS | |
| 2-G4_VL | EIVLTQSPGTLSLSPGERATLSCRASQSVSGSYLAWYQQKPGQAPRLLIYGASS | 19 |
| | RATGIPDRFSGSGSGTDFTLTISRLEPEDFAVYYCQQYGGSLPYTFGQGTKVEI | |
| | K | |
| 2-H2_VH | QVQLVESGGGVVQPGRSLRLSCAASGFTFSDYGMHWVRQAPGKGLEWVAVISYE | 20 |
| | GSNEYYADSVKGRFTISRDNSKNTLYLQMNSLRAEDTAVYYCARGSEGSGFDVW | |
| | GQGTLVTVSS | |
| 2-H2_VL | DIQMTQSPSSLSASVGDRVTITCRASQRISNYLNWYQQKPGKAPKLLIYAASSL | 21 |
| | QSGVPSRFSGSGSGTDFTLTISSLQPEDFATYYCQQSYSPPYTFGQGTKVEIK | |
| 2-H9_VH | EVQLLESGGGLVQPGGSLRLSCAASGFTFSTYAMSWVRQAPGKGLEWVSSIYGS | 22 |
| | GGKTYYADSVKGRFTISRDNSKNTLYLQMNSLRAEDTAVYYCARTDYSLYYQFD | |
| | YWGQGTLVTVSS | |
| 2-H9_VL | DIQMTQSPSSLSASVGDRVTITCRASQSISRYLNWYQQKPGKAPKLLIYAASSL | 23 |
| | QSGVPSRFSGSGSGTDFTLTISSLQPEDLATYYCQQSYSYPVTFGQGTKVEIK | |
| 3-A7_VH | QVQLVESGGGVVQPGRSLRLSCAASGFTFTNYGMHWVRQAPGKGLEWVAVISFD | 24 |
| | GSNKYYANSVKGRFTISRDNSKNTLYLQMNSLRAEDTAVYYCARGGDEDGFDVW | |
| | GQGTLVTVSS | |
| 3-A7_VL | DIQMTQSPSSLSASVGDRVTITCRASQSISSYLNWYQQKPGKAPKLLIYAASSL | 25 |
| | QSGVPSRFSGSGSGTDFTLTISSLQPEDFATYYCQQSYRPPYTFGQGTKVEIK | |
| 3-A9_VH | EVQLLESGGGLVQPGGSLRLSCAASGFTFSNYAMSWVRQAPGKGLEWVSSITGS | 26 |
| | GEYTNYADSVKGRFTISRDNSKNTLYLQMNSLRAEDTAVYYCAREVADYWDGGV | |
| | YYYYDGGFDVWGQGTLVTVSS | |
| 3-A9_VL | EIVLTQSPGTLSLSPGERATLSCRASQSVSSNYLAWYQQKPGQAPRLLIYGASS | 27 |
| | RATGIPDRFSGSGSGTDFTLTISRLEPEDFAVYYCQQYGRSAPITFGQGTKVEI | |
| | K | |
| 3-B3_VH | EVQLLESGGGLVQPGGSLRLSCAASGFTFSSYAMSWVRQAPGKGLEWVSTISGT | 28 |
| | GGRTYYADSVKGRFTISRDNSKNTLYLQMNSLRAEDTAVYYCARVDHYSGYAFD | |
| | LWGQGTLVTVSS | |
| 3-B3_VL | DIQMTQSPSSLSASVGDRVTITCRASQSISSYLNWYQQKPGKAPKLLIYAASSL | 29 |
| | QSGVPSRFSGSGSGTDFTLTISSLQPEDFATYYCQQSYSYPLTFGQGTKVEIK | |
| 3-C4_VH | EVQLLESGGGLVQPGGSLRLSCAASGFTFSSYAMSWVRQAPGKGLEWVSTIYGG | 30 |
| | GSYTYYADSVKGRFTISRDNSKNTLYLQMNSLRAEDTAVYYCARAEVDDYWYLY | |
| | MDVWGQGTLVTVSS | |
| 3-C4_VL | DIQMTQSPSSLSASVGDRVTITCRASQDIGNYLNWYQQKPGKAPKLLIYAASSL | 31 |
| | QSGVPSRFSGSGSGTDFTLTISSLQPEDFATYYCQQSYTFPLTFGQGTKVEIK | |
| 3-C6_VH | EVQLLESGGGLVQPGGSLRLSCAASGFTFSTYAMSWVRQAPGKGLEWVSTIYGS | 32 |
| | GGRTYYADSVKGRFTISRDNSKNTLYLQMNSLRAEDTAVYYCARTDHYSYYVMD | |
| | YWGQGTLVTVSS | |
| 3-C6_VL | DIQMTQSPSSLSASVGDRVTITCRASQSISKYLNWYQQKPGKAPKLLIYSASSL | 33 |
| | QSGVPSRFSGSGSGTDFTLTISSLQPEDCATYYCQQSYSFPLTFGQGTKVEIK | |
| 3-C8_VH | EVQLLESGGGLVQPGGSLRLSCAASGFTFSDYAMSWVRQAPGKGLEWVSAIYGS | 34 |
| | GSYTYYADSVKGRFTISRDNSKNTLYLQMNSLRAEDTAVYYCARTAYSYPYVYF | |
| | DFWGQGTLVTVSS | |
| 3-C8_VL | DIQMTQSPSSLSASVGDRVTITCRASQTITSYLNWYQQKPGKAPKLLIYAASSL | 35 |
| | QSGVPSRFSGSGSGTDFTLTISSLQPEDFATYYCQQSYTYPLTFGQGTKVEIK | |
| 3-C9_VH | EVQLLESGGGLVQPGGSLRLSCAASGFTFGNYAMSWVRQAPGKGLEWVSVISGG | 36 |
| | GTNTYYADSVKGRFTISRDNSKNTLYLQMNSLRAEDTAVYYCARSDSYYGVYDG | |
| | YYYGMDVWGQGTLVTVSS | |
| 3-C9_VL | DIQMTQSPSSLSASVGDRVTITCRASQTISRYLNWHQQKPGKAPKLLIYAASSL | 37 |
| | QSGVPSRFSGSGSGTDFTLTISSLQPEDFATYYCQQTYSTPWTFGQGTKVEIK | |
| 3- | EVQLLESGGGLVQPGGSLRLSCAASGFTFSSYAMSWVRQAPGKGLEWVSTIYAS | 38 |
| C11_VH | GATTYYADSVKGRFTISRDNSKNTLYLQMNSLRAEDTAVYYCARLKLYHAAFDI | |
| | WGQGTLVTVSS | |
| 3- | DIQMTQSPSSLSASVGDRVTITCRASQDISSYLNWYQQKPGKAPKLLIYTASSL | 39 |
| C11_VL | QSGVPSRFSGSGSGTDFTLTISSLQPEDFATYYCQQSYSYPLTFGQGTKVEIK | |
| 3- | EVQLLESGGGLVQPGGSLRLSCAASGFTFRDYAMSWVRQAPGKGLEWVSTIYGS | 40 |
| C12_VH | GSRTYYADSVKGRFTISRDNSKNTLYLQMNSLRAEDTAVYYCARVDHVHGYAFD | |
| | YWGQGTLVTVSS | |
| 3- | DIQMTQSPSSLSASVGDRVTITCRASQSISKYLNWYQQKPGKAPKLLIYAASSL | 41 |
| C12_VL | QSGVPSRFSGSGSGTDFTLTISSLQPEDFATYYCQQSYSFPPTFGQGTKVEIK | |
| 3-D3_VH | EVQLLESGGGLVQPGGSLRLSCAASGFTFSNYAMSWVRQAPGKGLEWVSTIYES | 42 |
| | GGYTYYADSVKGRFTISRDNSKNTLYLQMNSLRAEDTAVYYCARVESYGYGEYI | |
| | TYNYYGFDVWGQGTLVTVSS | |
| 3-D3_VL | EIVLTQSPGTLSLSPGERATLSCRASQSVATGYLAWYQQKPGQAPRLLIYGASS | 43 |
| | RATGIPDRFSGSGSGTDFTLTISRLEPEDFAVYYCQQYESSPPFTFGQGTKVEI | |
| | K | |
| 3-D6_VH | QVQLVQSGAEVKKPGSSVKVSCKASGGTFTSYGIHWVRQAPGQGLEWMGRIVPI | 44 |
| | LGTTNYAQKFQGRVTITADESTSTAYMELSSLRSEDTAVYYCARDPAVTLDTYV | |
| | YADHGFDVWGQGTLVTVSS | |
| 3-D6_VL | EIVMTQSPATLSVSPGERATLSCRASQSVYNNLAWYQQKPGQAPRLLIYGASTR | 45 |
| | ATGIPARFSGSGSGTEFTLTISSLQSEDFAVYYCQQYNDWPLTFGQGTKVEIK | |
| 3-D7_VH | EVQLLESGGGLVQPGGSLRLSCAASGFTFKNYAMSWVRQAPGKGLEWVSGISEG | 46 |
| | GANTYYADSVKGRFTISRDNSKNTLYLQMNSLRAEDTAVYYCAREPPKYGAYSY | |
| | YGYGFDPWGQGTLVTVSS | |
| 3-D7_VL | EIVLTQSPGTLSLSPGERATLSCRASQSVGSNYLAWYQQKPGQAPRLLIYGASS | 47 |
| | RATGIPDRFSGSGSGTDFTLTISRLEPEDFAVYYCQQYGSSPYTFGQGTKVEIK | |
| 3- | EVQLLESGGGLVQPGGSLRLSCAASGFTFKGYAMSWVRQAPGKGLEWVSSISVS | 48 |
| D12_VH | GAGTYYADSVKGRFTISRDNSKNTLYLQMNSLRAEDTAVYYCARYVRGVSFDVW | |
| | GQGTLVTVSS | |
| 3- | EIVLTQSPGTLSLSPGERATLSCRASQSVSSSYLAWYQQKPGQAPRLLIYGASS | 49 |
| D12_VL | RATGIPDRFSGSGSGTDFTLTISRLEPEDFAVYYCQQYETSPPVTFGQGTKVEI | |
| | K | |
| 3-E2_VH | EVQLLESGGGLVQPGGSLRLSCAASGFTFSNYAMSWVRQAPGKGLEWVSAIGGS | 50 |
| | GGGTYYADSVKGRFTISRDNSKNTLYLQMNSLRAEDTAVYYCARYGWEYQAFDY | |
| | WGQGTLVTVSS | |
| 3-E2_VL | DIQMTQSPSSLSASVGDRVTITCRASQSISRYLNWYQQKPGKAPKLLIYAASSL | 51 |
| | QSGVPSRFSGSGSGTDFTLTISSLQPEDFATYYCQQSYSTPWTFGQGTKVEIK | |
| 3-E5_VH | EVQLLESGGGLVQPGGSLRLSCAASGFTFSNYAMSWVRQAPGKGLEWVSGITAG | 52 |
| | GGNTYYADSVKGRFTISRDNSKNTLYLQMNSLRAEDTAVYYCARIAGLFDGFDV | |
| | WGQGTLVTVSS | |
| 3-E5_VL | EIVLTQSPGTLSLSPGERATLSCRASQSVRSSYLAWYQQKPGQAPRLLIYGASS | 53 |
| | RATGIPDRFSGSGSGTDFTLTISRLEPEDFAVYYCQQYASLPLTFGQGTKVEIK | |
| 3-E7_VH | EVQLLESGGGLVQPGGSLRLSCAASGFTFSNYAMSWVRQAPGKGLEWVSGISAS | 54 |
| | GGGTYYADSVKGRFTISRDNSKNTLYLQMNSLRAEDTAVYYCARSSRYGDTFDV | |
| | WGQGTLVTVSS | |
| 3-E7_VL | EIVMTQSPATLSVSPGERATLSCRASLSVGSNLAWYQQKPGQAPRLLIYGASTR | 55 |
| | ATGIPARFSGSGSGTEFTLTISSLQSEDFAVYYCQQYNNLPLTFGQGTKVEIK | |
| 3-E8_VH | EVQLLESGGGLVQPGGSLRLSCAASGFTFTDYAMSWVRQAPGKGLEWVSGISVS | 56 |
| | GGYTYYADSVKGRFTISRDNSKNTLYLQMNSLRAEDTAVYYCARGLYAHHTYYY | |
| | GGFDYWGQGTLVTVSS | |
| 3-E8_VL | EIVMTQSPATLSVSPGERATLSCRASQSVGSNLAWYQQKPGQAPRLLIYGASTR | 57 |
| | ATGIPARFSGSGSGTEFTLTISSLQSEDFAVYYCQQYHNWPPTFGQGTKVEIK | |
| 3-E9_VH | EVQLLESGGGLVQPGGSLRLSCAASGFTFRSYAMSWVRQAPGKGLEWVSTIYGT | 58 |
| | GEDTYYADSVKGRFTISRDNSKNTLYLQMNSLRAEDTAVYYCARDYFWSPRGSI | |
| | YDYDYFDVWGQGTLVTVSS | |
| 3-E9_VL | DIQMTQSPSSLSASVGDRVTITCRASQSISRYLNWYQQKPGKAPKLLIYSASSL | 59 |
| | QSGVPSRFSGSGSGTDFTLTISSLQPEDFATYYCQQYFSAPYTFGQGTKVEIK | |
| 3-F5_VH | EVQLLESGGGLVQPGGSLRLSCAASGFTFKGYAMSWVRQAPGKGLEWVSSISVS | 60 |
| | GAGTYYADSVKGRFTISRDNSKNTLYLQMNSLRAEDTAVYYCARYVRGVSFDVW | |
| | GQGTLVTVSS | |
| 3-F5_VL | EIVLTQSPGTLSLSPGERATLSCRASQSVSSNYLAWYQQKPGQAPRLLIYGASS | 61 |
| | RATGIPDRFSGSGSGTDFTLTISRLEPEDFAVYYCQQYGSSPAVTFGQGTKVEI | |
| | K | |
| 3-F6_VH | EVQLLESGGGLVQPGGSLRLSCAASGFTFSDYAMSWVRQAPGKGLEWVSAISSS | 62 |
| | GGTTYYADSVKGRFTISRDNSKNTLYLQMNSLRAEDTAVYYCARDAGWSYYGLW | |
| | IYYYYMDVWGQGTLVTVSS | |
| 3-F6_VL | EIVLTQSPGTLSLSPGERATLSCRASQSVRSNYLAWYQQKPGQAPRLLIYGASS | 63 |
| | RATGIPDRFSGSGSGTDFTLTISRLEPEDFAVYYCQQYGASPQTFGQGTKVEIK | |
| 3- | EVQLLESGGGLVQPGGSLRLSCAASGFTFKDYAMSWVRQAPGKGLEWVSGISGS | 64 |
| F12_VH | GGYTYYADSVKGRFTISRDNSKNTLYLQMNSLRAEDTAVYYCARKLGGFDENYT | |
| | YGMDVWGQGTLVTVSS | |
| 3- | EIVLTQSPGTLSLSPGERATLSCRASQSVSSSYLAWYQQKPGQAPRLLIYGASS | 65 |
| F12_VL | RATGIPDRFSGSGSGTDFTLTISRLEPEDFAVYYCQQYGSSPEYTFGQGTKVEI | |
| | K | |
| 3-G4_VH | EVQLLESGGGLVQPGGSLRLSCAASGFTFSTYAMSWVRQAPGKGLEWVSGISAG | 66 |
| | GGARTYYADSVKGRFTISRDNSKNTLYLQMNSLRAEDTAVYYCARVHHFGYYSL | |
| | DVWGQGTLVTVSS | |
| 3-G4_VL | DIQMTQSPSSLSASVGDRVTITCRASQTISNYLNWYQQKPGKAPKLLIYAASSL | 67 |
| | QSGVPSRFSGSGSGTDFTLTISSLQPEDFATYYCQQSYTYPLTFGQGTKVEIK | |
| 3-G5_VH | EVQLLESGGGLVQPGGSLRLSCAASGFTFSSYAMSWVRQAPGKGLEWVSVIYGS | 68 |
| | GGRTYYADSVKGRFTISRDNSKNTLYLQMNSLRAEDTAVYYCARHDPRYQLDVW | |
| | GQGTLVTVSS | |
| 3-G5_VL | DIQMTQSPSSLSASVGDRVTITCRASQSIGSYLNWYQQKPGKAPKLLIYTASSL | 69 |
| | QSGVPSRFSGSGSGTDFTLTISSLQPEDFATYYCQQSYTYPLTFGQGTKVEIK | |
| 3-G6_VH | QVQLVESGGGVVQPGRSLRLSCAASGFTFSDYGMHWVRQAPGKGLEWVAVISYD | 70 |
| | GSRKYYANSVKGRFTISRDNSKNTLYLQMNSLRAEDTAVYYCARGSYSDGFDVW | |
| | GQGTLVTVSS | |
| 3-G6_VL | DIQMTQSPSSLSASVGDRVTITCRASQSISRYLNWYQQKTGKAPKLLIYGASSL | 71 |
| | QSGVPSRFSGSGSGTDFTLTISSLQPEDFATYYCQQSYSTPYTFGQGTKVEIK | |
| 3-G7_VH | QVQLVQSGAEVKKPGSSVKVSCKASGGTFSTEYISWVRQAPGQGLEWMGRIIPV | 72 |
| | LGITNYAQKFQGRVTITADESTSTAYMELSSLRSEDTAVYYCARHLGPPYLVTY | |
| | SHGFDVWGQGTLVTVSS | |
| 3-G7_VL | EIVMTQSPATLSVSPGERATLSCRASQSLGTNLAWYQQKPGQAPRLLIYGASTR | 73 |
| | ATGIPARFSGSGSGTEFTLTISSLQSEDFAVYYCQQYYEAPPTFGQGTKVEIK | |
| 3-G8_VH | EVQLLESGGGLVQPGGSLRLSCAASGFTFSNYAMSWVRQAPGKGLEWVSAIYGS | 74 |
| | GGKTYYADSVKGRFTISRDNSKNTLYLQMNSLRAEDTAVYYCARLKYGFYQFDY | |
| | WGQGTLVTVSS | |
| 3-G8_VL | DIQMTQSPSSLSASVGDRVTITCRASQSIRNYLNWYEQKPGKAPKLLIYAASSL | 75 |
| | QSGVPSRFSGSGSGTDFTLTISSLQPEDFATYYCQQSYSYPLTFGQGTKVEIK | |
| 3-H1_VH | EVQLLESGGGLVQPGGSLKLSCAASGFTFSNYAMSWVRQAPGKGLEWVSVIYAG | 76 |
| | GARTYYADSVKGRFTISRDNSKNTLYLQMNSLRAEDTAVYYCARWGGDGFYAMD | |
| | YWGQGTLVTVSS | |
| 3-H1_VL | DIQMTQSPSSLSASVGDRVTITCRASQTISTYLNWYQQKPGKAPKLLIYAASIL | 77 |
| | QSGVPSRFSGSGSGTDFTLTISSLQPEDFATYYCQQSYTVPLTFGQGTKVEIK | |
| 3-H4_VH | EVQLLESGGGLVQPGGSLRLSCAASGFTFSNYAMSWVRQAPGKGLEWVSAISGS | 78 |
| | GGTTYYADSVKGRFTISRDNSKNTLYLQMNSLRAEDTAVYYCARIKKYGSAGYT | |
| | VGFDSWGQGTLVTVSS | |
| 3-H4_VL | EIVMTQSPATLSVSPGERATLSCRASQSVGSYLAWYQQKPGQAPRLLIYGASTR | 79 |
| | ATGIPARFSGSGSGTEFTLTISSLQSEDFAVYYCQQYYNAPITFGQGTKVEIK | |
| 4-A7_VH | QLQLQESGPGLVKPSETLSLTCTVSGGSISSSSYYWGWIRQPPGKGLEWIGNIY | 80 |
| | YTGTTYYNPSLKSRVTISVDTSKNQFSLKLSSVTAADTAVYYCARIKVPWYYSS | |
| | YYFDYWGQGTLVTVSS | |
| 4-A7_VL | QSVLTQPPSVSGAPGQRVTISCTGSSSNIGAGYDVHWYQQLPGTAPKLLIYGNK | 81 |
| | NRPSGVPDRFSGSKSGTSASLAITGLQAEDEADYYCQSYDTSLSAWVFGGGTKL | |
| | TVL | |
| 4-A8_VH | EVQLLESGGGLVQPGGSLRLSCAASGFTFKNYAMSWVRQAPGKGLEWVSAISAS | 82 |
| | GGYTYYADSVKGRFTISRDNSKNTLYLQMNSLRAEDTAVYYCARGRWGAFDYWG | |
| | QGTLVTVSS | |
| 4-A8_VL | QSVLTQPPSVSGAPGQRVTISCTGSSSNIGSGYDVHWYQQLPGTAPKLLIYGNR | 83 |
| | NRPSGVPDRFSGSKSGTSASLAITGLQAEDEADYYCQSYDSSLKTWVFGGGTKL | |
| | TVL | |
| 4-B1_VH | EVQLLESGGGLVQPGGSLKLSCAASGFTFSDYAMSWVRQAPGKGLEWVSGISRG | 84 |
| | GARTYYADSVKGRFTISRDNSKNTLYLQMNSLRAEDTAVYYCARYLFGHYMDWG | |
| | QGTLVTVSS | |
| 4-B1_VL | QSVLTQPPSVSGAPGQRVTISCTGSSSNIGAGYDVHWYQQLPGTAPKLLIYGNT | 85 |
| | NRPSGVPDRFSGSKSGTSASLAITGLQAEDEADYYCQSYDGRLGVSVFGGGTKL | |
| | TVL | |
| 4-B2_VH | QVQLVESGGGVVQPGRSLRLSCAASGFTFSRYAMHWVRQAPGKGLEWVAVISHS | 86 |
| | GSTKYYANSVKGRFTISRDNSKNTLYLQMNSLRAEDTAVYYCARSRGWRGFDSW | |
| | GQGTLVTVSS | |
| 4-B2_VL | QSVLTQPPSVSGAPGQRVTISCTGSSSNIGAYVVHWYQQLPGTAPKLLIYGNTN | 87 |
| | RPSGVPDRFSGSKSGTSASLAITGLQAEDEADYYCQSYDRTLSSYVFGGGTKLT | |
| | VL | |
| 4- | QVQLVESGGGVVQPGRSLRLSCAASGFAFSGYGMHWVRQAPGKGLEWVAVISHH | 88 |
| B11_VH | GSYKYYANSVKGRFTISRDNSKNTLYLQMNSLRAEDTAVYYCARGGADVGYFYS | |
| | TFYYYMDVWGQGTLVTVSS | |
| 4- | QSVLTQPPSVSGAPGQRVTISCTGSSSNIGAAFDVHWYQQLPGTAPKLLIYDNY | 89 |
| B11_VL | NRPSGVPDRFSGSKSGTSASLAITGLQAEDEADYYCQSYDSSLSVWVFGGGTKL | |
| | TVL | |
| 4- | EVQLLESGGGLVQPGGSLRLSCAASGFTFSNYAMSWVRQAPGKGLEWVSAISRG | 90 |
| D12_VH | GGYTYYADSVKGRFTISRDNSKNTLYLQMNSLRAEDTAVYYCARWGGDGFYAMD | |
| | YWGQGTLVTVSS | |
| 4- | QSVLTQPPSVSGAPGQRVTISCTGSSSNIGAGYDVHWYQQLPGTAPKLLIYANN | 91 |
| D12_VL | NRPSGVPDRFSGSKSGTSASLAITGLQAEDEADYYCQSYDYSLSGWVFGGGTKL | |
| | TVL | |
| 4-F4_VH | QLQLQESGPGLVKPSETLSLTCTVSGGSISSRGYYWAWIKQPPGKGLEWIGSIY | 92 |
| | YSGSTYYNPSLKSRVTISVDTSKNQFSLKLSSVTAADTAVYYCARLVSYGAYYV | |
| | FDYWGQGTLVTVSS | |
| 4-F4_VL | QSVLTQPPSVSGAPGQRVTISCTGSSSNIGAGYDVHWYQQLPGTAPKLLIYGNR | 93 |
| | NRPSGVPDRFSGSKSGTSASLAITGLQAEDEADYYCQSYDTRLSAWVFGGGTKL | |
| | TVL | |
| 4- | EVQLLESGGGLVRPGGSLRLSCAASGFTFSSYAMSWVRQAPGKGLEWVSAISGS | 94 |
| G12_VH | GGSTYYADSVKGRFTISRDNSKNTLYLQMNSLRAEDTAVYYCARDKRLTYWGQG | |
| | TMVTVSS | |
| 4- | QSVVTQPPSVSAAPGQKVTISCSGSSSNIGNNYVSWYQQLPGTAPKLLIYDNNK | 95 |
| G12_VL | RPSGIPDRFSGSKSGTSATLGITGLQTGDEADYYCGTWDSSLSARVFGGGTKVT | |
| | VL | |
| 5-C9_VH | QVQLVQSGAEVKKPGSSVKVSCKASGGTFSSYAINWVRQAPGQGLEWMGRIIPL | 96 |
| | LETADYAQKFQGRVTITADESTSTAYMELSSLRSEDTAVYYCAREWPGEYFDVW | |
| | GQGTLVTVSS | |
| 5-C9_VL | EIVLTQSPGTLSLSPGERATLSCRASQSVASSYLAWYQQKPGQAPRLLIYGASS | 97 |
| | RATGIPDRFSGSGSGTDFTLTISRLEPEDFAVYYCQQYGSSQLTFGQGTKVEIK | |
| 5-E2_VH | QVQLVESGGGVVQPGRSLRLSCAASGFTFRDYGMHWVRQAPGKGLEWVAVISYH | 98 |
| | GRNEYYANSVKGRFTISRDNSKNTLYLQMNSLRAEDTAVYYCARGAYGDDFDVW | |
| | GQGTLVTVSS | |
| 5-E2_VL | DIQMTQSPSSLSASVGDRVTITCRASQSISSYLNWYQQKTGKAPKLLIYAASSL | 99 |
| | QSGVPSRFSGSGSGTDFTLTISSLQPEDFATYYCQQSYSAPYTFGQGTKVEIK | |
| 5- | EVQLLESGGGLVQPGGSLRLSCAASGFTFSNYAMSWVRQAPGKGLEWVSTISGG | 100 |
| E12_VH | GRTTNYADSVKGRFTISRDNSKNTLYLQMNSLRAEDTAVYYCARSTPIPSYYPY | |
| | IYSYYFDVWGQGTLVTVSS | |
| 5- | EIVMTQSPATLSVSPGERATLSCRASQSVSNNLAWYQQKPGQAPRLLIYGASTR | 101 |
| E12_VL | ATGIPARFSGSGSGTEFTLTISSLQSEDFAVYYCQQYGNYPPTFGQGTKVEIK | |
| 6-G4_VH | EVQLLESGGGLVQPGGSLRLSCAASGFTFSDYAMSWVRQAPGKGLEWVSSISSS | 102 |
| | GANTYYADSGKGRFTISRDNSKNILYLQMNSLRAEDTAVYYCARSVVTWVTYAF | |
| | DYWGQGTLVTVSS | |
| 6-G4_VL | QSVLTQPPSVSGAPGQRVTISCTGSSSNIGAGYDVHWYQQLPGTAPKLLIYDNT | 103 |
| | NRPSGVPDRFSGSKSGTSASLAITGLQAEDEADYYCQSYDSGLSGWVFGGGTKL | |
| | TVL | |
| 6-H5_VH | EVQLLESGGGLVQPGGSLRLSCAASGFTFSNYAMSWVRQAPGKGLEWVSAISAT | 104 |
| | GGATYYADSVKGRFTISRDNSKNTLYLQMNSLRAEDTAVYYCARSVRERYTYYM | |
| | DYWGQGTLVTVSS | |
| 6-H5_VL | QSVLTQPPSVSGAPGQRVTISCTGSSSNIGAGYDVHWYQQLPGTAPKLLIYDNT | 105 |
| | NRPSGVPDRFSGSKSGTSASLAITGLQAEDEADYYCQSYDVSLGVWVFGGGTKL | |
| | TVL | |
| 8-A2_VH | QVQLVQSGAEVKKPGSSVKVSCKASGGTFSNYYISWVRQAPGQGLEWMGGIIPV | 106 |
| | FGVAHYAQKFQGRVTITADESTSTAYMELSSLRSEDTAVYYCARGYPGEYFDYW | |
| | GQGTLVTVSS | |
| 8-A2_VL | EIVLTQSPGTLSLSPGERATLSCRASQSVSSSYLAWYQQKPGQAPRLLIYAASS | 107 |
| | RATGIPDRFSGSGSGTDFTLTISRLEPEDFAVYYCQHYGGSPGYTFGQGTKVEI | |
| | K | |
| 8-A4_VH | EVQLLESGGGLVQPGGSLRLSCAASGFTFGSYAMSWVRQAPGKGLEWVSSIYAG | 108 |
| | GGRTYYADSVKGRFTISRDNSKNTLYLQMNSLRAEDTAVYYCARWGGDGFYAMD | |
| | YWGQGTLVTVSS | |
| 8-A4_VL | DIQMTQSPSSLSASVGDRVTITCRASQTISNYLNWYQQKPGKAPKLLIYGASSL | 109 |
| | QSGVPSRFSGSGSGTDFTLTISSLQPEDFATYYCQQSFTFPLTFGQGTKVEIK | |
| 8-A6_VH | EVQLLESGGGLVQPGGSLRLSCAASGFTFSDYAMSWVRQAPGKGLEWVSTIYSG | 110 |
| | GVRTYYADSVKGRFTISRDNSKNTLYLQMNSLRAEDTAVYYCARGLVSFRGYAF | |
| | DYWGQGTLVTVSS | |
| 8-A6_VL | DIQMTQSPSSLSASVGDRVTITCRASQSISSYLNWYQQKPGKAPKLLIYAASSL | 111 |
| | QSGVPSRFSGSGSGTDFTLTISSLQPEDFATYYCQQSYTIPPTFGQGTKVEIK | |
| 8-A7_VH | EVQLLESGGGLVQPGGSLRLSCAASGFTFRNYAMSWVRQAPGKGLEWVSSISGG | 112 |
| | GINTYYADSVKGRFTISRDNSKNTLYLQMNSLRAEDTAVYYCARVRSYSGPGYF | |
| | DYWGQGTLVTVSS | |
| 8-A7_VL | EIVMTQSPATLSVSPGDRATLSCRASRSVSSNLAWYQQKPGQAPRLLIYGASTR | 113 |
| | ATGIPARFSGSGSGTEFTLTISSLQSEDFAVYYCQQYYNWPLTFGQGTKVEIK | |
| 8-A8_VH | EVQLLESGGGLVQPGGSLRLSCAASGFTFSNYAMSWVRQAPGKGLEWVSVIYGS | 114 |
| | GARTYYADSVKGRFTISRDNSKNTLYLQMNSLRAEDTAVYYCARISPHYDFDVW | |
| | GQGTLVTVSS | |
| 8-A8_VL | DIQMTQSPSSLSASVGDRVTITCRASQTISKYLNWYQQKPGKAPKLLIYAASSL | 115 |
| | QSGVPSRFSGSGSGTDFTLTISSLQPEDFATYYCQQSYSYPITFGQGTKVEIK | |
| 8-A9_VH | EVQLLESGGGLVQPGGSLRLSCAASGFTFSNYAMSWVRQAPGKGLEWVSAISGG | 116 |
| | GSRTYYADSVKGRFTISRDNSKNTLYLQMNSLRAEDTAVYYCAREARVLGYSLD | |
| | YWGQGTLVTVSS | |
| 8-A9_VL | DIQMTQSPSSLSASVGDRVTITCRASQRIGKYLNWYQQKPGKAPKLLIYAASSL | 117 |
| | QSGVPSRFSGSGSGTDFTLTISSLQPEDFATYYCQQSYSYPLTFGQGTKVEIK | |
| 8-C4_VH | EVQLLESGGGLVQPGGSLRLSCAASGFTFSNYAMSWVRQAPGKGLEWVSAIYGS | 118 |
| | GGRTYYADSVKGRFTISRDNSKNTLYLQMNSLRAEDTAVYYCARLRYGFYQFDY | |
| | WGQGTLVTVSS | |
| 8-C4_VL | DIQMTQSPSSLSASVGDRVTITCRASQSIRNYLNWYEQKPGKAPKLLIYAASSL | 119 |
| | QSGVPSRFSGSGSGTDFTLTISSLQPEDFATYYCQQSYSYPLTFGQGTKVEIK | |
| 8-C6_VH | EVQLLESGGGLVQPGGSLRLSCAASGFTFTDYAMSWVRQAPGKGLEWVSTIYSS | 120 |
| | GHRTYYADSVKGRFTISRDNSKNTLYLQMNSLRAEDTAVYYCARLYPLHSYAFD | |
| | HWGQGTLVTVS | |
| 8-C6_VL | DIQMTQSHSSLSASVGDRVTITCRASQSISRYLNWYQQKPGKAPKLLIYAASSL | 121 |
| | QSGVPSRFSGSGSGTDFTLTISSLQPEDFATYYCQQSYSVPLTFGQGTKVEIK | |
| 8-C9_VH | EVQLLESGGGLVQPGGSLRLSCAASGFTFKSYAMSWVRQAPGKGLEWVSLISSS | 122 |
| | GENTYYADSVKGRFISRDNSKNTLYLQMNSLRAEDTAVYYCARVRFGYGSWRY | |
| | RKYMDVWGQGTLVTVSS | |
| 8-C9_VL | DIQMTQSPSSLSASVGDRVTITCRASQSIATYLNWYQQKPGKAPKLLIYGASSL | 123 |
| | QSGVPSRFSGSGSGTDFTLTISSLQPEDFATYYCQQSYSTPLTFGQGTKVEIK | |
| 8- | EVQLLESGGGLVQPGGSLRLSCAASGFTFSSYAMSWVRQAPGKGLEWVSTISAS | 124 |
| C10_VH | GATYYADSVKGRFTISRDNSKNTLYLQMNSLRAEDTAVYYCARGPYSYYYYSFD | |
| | YWGQGTLVTVSS | |
| 8- | DIQMTQSPSSLSASVGDRVTITCRASQTISTYLNWYQQKPGKAPKLLIYAASSL | 125 |
| C10_VL | QSGVPSRFSGSGSGTDFTLTISSLQPEDFATYYCQQSYRYPLTFGQGTKVEIK | |
| 8- | EVQLLESGGGLVQPGGSLRLSCAASGFTFSSYAMSWVRQAPGKGLEWVSTIYVG | 126 |
| C11_VH | GHRTYYADSVKGRFTISRDNSKNTLYLQMNSLRAEDTAVYYCARVGWSWGFDPW | |
| | GQGTLVTVSS | |
| 8- | DIQMTQSPSSLSASVGDRVTITCRASQTISKYLNWYQQKPGKAPKLLIYSASSL | 127 |
| C11_VL | QSGVPSRFSGSGSGTDFTLTISSLQPEDFATYYCQQSYTYPLTFGQGTKVEIK | |
| 8- | EVQLVESGGGLVKPGGSLRLSCAASGFTFTNAWMSWVRQAPGKGLEWVGRIKSI | 128 |
| D11_VH | TEGGTTDYAAPVKGRFTISRDDSKNTLYLQMNSLKTEDTAVYYCARVWHTDDYY | |
| | EGFDVWGQGTLVTVSS | |
| 8- | EIVLTQSPGTLSLSPGERATLSCRASQSVSGSYLAWYQQKPGQAPRLLIYDASS | 129 |
| D11_VL | RATGIPDRFSGSGSGTDFTLTISRLEPEDFAVYYCQQYGTTPQTFGQGTKVEIK | |
| 8-D4_VH | EVQLLESGGGLVQPGGSLRLSCAASGFTFRSYAMSWVRQAPGKGLEWVSTIYGS | 130 |
| | GARTYYADSVKGRFTISRDNSKNTLYLQMNSLRAEDTAVYYCARWGGDGFYAMD | |
| | YWGQGTLVTVSS | |
| 8-D4_VL | DIQMTQSPSSLSASVGDRVTITCRASQSIYTYLNWYQQKPGKAPKLLIYAASSL | 131 |
| | QSGVPSRFSGSGSGTDFTLTISSLQPEDFATYYCQQSYSFPLTFGQGTKVEIK | |
| 8-D7_VH | EVQLLESGGGLVQPGGSLRLSCAASGFTFSSYAMSWVRQAPGKGLEWVSGISTS | 132 |
| | GGTTYYADSVKGRFTISRDNSKNTLYLQMNSLRAEDTAVYYCARVVGRAVFDIW | |
| | GQGTLVTVSS | |
| 8-D7_VL | EIVLTQSPGTLSLSPGERATLSCRASQSVSSTYLAWYQQKPGQAPRLLIYAASS | 133 |
| | RATGIPDRFSGSGSGTDFTLTISRLEPEDFAVYYCQQYAALPITFGQGTKVEIK | |
| 8-E2_VH | QVQLVQSGAEVKKPGSSVKVSCKASGGTFSDEAIHWVRQAPGQGLEWMGRIIPV | 134 |
| | LGIASYAQKFQGRVTITADESTSTAYMELSSLRSEDTAVYYCARGLSRKAFYFD | |
| | YWGQGTLVTVSS | |
| 8-E2_VL | DIQMTQSPSSLSASVGDRVTITCRASQTIGNYLNWYQQKPGKAPKLLIYVASSL | 135 |
| | QSGVPSRFSGSGSGTDFTLTISSLQPEDFATYYCQQSYRYPYTFGQGTKVEIK | |
| 8-E3_VH | EVQLLESGGGLVQPGGSLRLSCAASGFTFSNYAMSWVRQAPGKGLEWVSTIYRG | 136 |
| | AGTYYADSVKGRFTISRDNSKNTLYLQMNSLRAEDTAVYYCARDASWYGPFSYY | |
| | YYYGFDVWGQGTLVTVSS | |
| 8-E3_VL | EIVMTQSPATLSVSPGERATLSCRASQSVYTNLAWYQQKPGQAPRLLIYDASTR | 137 |
| | ATGIPARFSGSGSGTEFTLTISSLQSEDFAVYYCQQYNDWPLTFGQGTKVEIK | |
| 8-E9_VH | EVQLLESGGGLVQPGGSLRLSCAASGFTFSDYAMSWVRQAPGKGLEWVSIITES | 138 |
| | GVNTYYADSVKGRFTISRDNSKNTLYLQMNSLRAEDTAVYYCARHPSVGSASRS | |
| | YYFDVWGQGTLVTVSS | |
| 8-E9_VL | EIVLTQSPGTLSLSPGERATLSCRASQSVGSSYLAWYQQKPGQAPRLLIYAASS | 139 |
| | RATGIPDRFSGSGSGTDFTLTISRLEPEDFAVYYCQQYYSAPLTFGQGTKVEIK | |
| 8-F1_VH | EVQLLESGGGLVQPGGSLRLSCAASGFTFTDYAMSWVRQAPGKGLEWVSGISGS | 140 |
| | GGVGTYYADSVKGRFTISRDNSKNTLYLQMNSLRAEDTAVYYCARGEYGSSIAY | |
| | SYYYGFDVWGQGTLVTVSS | |
| 8-F1_VL | EIVMTQSPATLSVSPGERATLSCRASQSVYNNLAWYQQKPGQAPRLLIYDASTR | 141 |
| | ATGIPARFSGSGSGTEFTLTISSLQSEDFAVYYCQQYNDWPYTFGQGTKVEIK | |
| 8-F2_VH | EVQLLESGGGLVQPGGSLRLSCAASGFTFSNYAMSWVRQAPGKGLEWVSAISGG | 142 |
| | GSRTYYADSVKGRFTISRDNSKNTLYLQMNSLRAEDTAVYYCAREARVLGYSLD | |
| | YWGQGTLVTVSS | |
| 8-F2_VL | DIQMTQSPSSLSASVGDRVTITCRASQRIGKYLNWYQQKPGKAPKLLIYAASSL | 143 |
| | QSGVPSRFSGSGSGTDFTLTISSLQPEDFATYYCQQSYSYPLTFGQGTKVEIK | |
| 8-F4_VH | EVQLLESGGGLVQPGGSLRLSCAASGFTFSGYAMSWVRQAPGKGLEWVSAIRGG | 144 |
| | GGTYYADSVKGRFTISRDNSKNTLYLQMNSLRAEDTAVYYCARVGPLRLYGFDY | |
| | WGQGTLVTVSS | |
| 8-F4_VL | DIQMTQSPSSLSASVGDRVTITCRASQSISNYLNWYQQKPGKAPKLLIYAASSL | 145 |
| | QSGVPSRFSGSGSGTDFTLTISSLQPEDFATYYCQQSYSYPLTFGQGTKVEIK | |
| 8-F6_VH | EVQLLESGGGLVQPGGSLRLSCAASGFTFSSYAMSWVRQAPGKGLEWVSVISGS | 146 |
| | GGNTYYADSVKGRFTISRDNSKNTLYLQMNSLRAEDTAVYYCAREVRHDSYYYY | |
| | YYSGMDVWGQGTLVTVSS | |
| 8-F6_VL | EIVMTQSPATLSVSPGERATLSCRASQSVYNNLAWYQQKPGQAPRLLIYDASTR | 147 |
| | ATGIPARFSGSGSGTEFTLTISSLQSEDFAVYYCQQYSDYPFTFGQGTKVEIK | |
| 8- | QVQLVQSGAEVKKPGSSVKVSCKASGGTFSNYAINWVRQAPGQGLEWMGRIIPT | 148 |
| F11_VH | LGIANYAQKFQGRVTITADESTSTAYMELSSLRSEDTAVYYCARAAYFAGPAAR | |
| | YGYFDIWGQGTLVTVSS | |
| 8- | EIVLTQSPGTLSLSPGERATLSCRASQSVRSNYLAWYQQKPGQAPRLLIYDASS | 149 |
| F11_VL | RATGIPDRFSGSGSGTDFTLTISRLEPEDFAVYYCQQYAYAPWTFGQGTKVEIK | |
| 8- | EVQLLESGGGLVQPGGSLRLSCAASGFTFRGYAMSWVRQAPGKGLEWVSSISVS | 150 |
| G12_VH | GAGTYYADSVKGRFTISRDNSKNTLYLQMNSLRAEDTAVYYCARYVRGVSFDVW | |
| | GQGTLVTVSS | |
| 8- | EIVLTQSPGTLSLSPGERATLSCRASQSVSSNYLAWYQQKPGQAPRLLIYGASS | 151 |
| G12_VL | RATGIPDRFSGSGSGTDFTLTISRLEPEDFAVYYCQQYGSSPAVTFGQGTKVEI | |
| | K | |
| 8-H7_VH | EVQLLESGGGLVQPGGSLRLSCAASGFTFSNYAMSWVRQAPGKGLEWVSAIYGS | 152 |
| | GGRTYYADSVKGRFTISRDNSKNTLYLQMNSLRAEDTAVYYCARLRYGFYQFDY | |
| | WGQGTLVTVSS | |
| 8-H7_VL | DIQMTQSPSSLSASVGDRVTITCRASQSIRNYLNWYEQKPGKAPKLLIYAASSL | 153 |
| | QSGVPSRFSGSGSGTDFTLTISSLQPEDFATYYCQQSYSYPLTFGQGTKVEIK | |
| 9-A6_VH | EVQLLESGGGLVQPGGSLRLSCAASGFTFSDYAMSWVRQAPGKGLEWVSTIYSG | 154 |
| | GVRTYYADSVKGRFTISRDNSKNTLYLQMNSLRAEDTAVYYCARGLVSFRGYAF | |
| | DYWGQGTLVTVSS | |
| 9-A6_VL | DIQMTQSPSSLSASVGDRVTITCRASQSISSYLNWYQQKPGKAPKLLIYAASSL | 155 |
| | QSGVPSRFSGSGSGTDFTLTISSLQPEDFATYYCQQSYTIPPTFGQGTKVEIK | |
| 9-B3_VH | QVQLVESGGGVVQPGRSLRLSCAASGFTFSDYGMHWVRQAPGKGLEWVAVISYD | 156 |
| | GSRKYYANSVKGRFTISRDNSKNTLYLQMNSLRAEDTAVYYCARGSYSDGFDVW | |
| | GQGTLVTVSS | |
| 9-B3_VL | DIQMTQSPSSLSASVGDRMTITCRASQSISNYLNWYQQKPGKAPKLLIYAASSL | 157 |
| | QSGVPSRFSGSGSGTDFTLTISSLQPEDFATYYCQQSYAPPYTFGQGTKVEIK | |
| 9- | EVQLLESGGGLVQPGGSLRLSCAASGFTFSSYAMSWVRQAPGKGLEWVSGISES | 158 |
| B10_VH | GGGTYYADSVKGRFTISRDNSKNTLYLQMNSLRAEDTAVYYCARLVPWQLAFDV | |
| | WGQGTLVTVSS | |
| 9- | EIVMTQSPATLSVSPGERATLSCRASQSVYNNLAWYQQKPGQAPRLLIYDASTR | 159 |
| B10_VL | ATGIPARFSGSGSGTEFTLTISSLQSEDFAVYYCQQYYYWPITFGQGTKVEIK | |
| 9-C2_VH | EVQLLESGGGLVQPGGSLRLSCAASGFTFSSYAMSWVRQAPGKGLEWVSVISGS | 160 |
| | GGNTYYADSVKGRFTISRDNSKNTLYLQMNSLRAEDTAVYYCAREVRHDSYYYY | |
| | YYSGMDVWGQGTLVTVSS | |
| 9-C2_VL | EIVMTQSPATLSVSPGERATLSCRASQSVYNNLAWYQQKPGQAPRLLIYDASTR | 161 |
| | ATGIPARFSGSGSGTEFTLTISSLQSEDFAVYYCQQYSDYPFTFGQGTKVEIK | |
| 9-D8_VH | QVQLVQSGAEVKKPGSSVKVSCKASGGTFSSEAISWVRQAPGQGLEWMGRIIPI | 162 |
| | SGRPNYAQKFQGRVTITADESTSTAYMELSSLRSEDTAVYYCAREASYYVAGYY | |
| | RYYGFDVWGQGTLVTVSS | |
| 9-D8_VL | EIVMTQSPATLSVSPGERATLSCRASRSLSNNLAWYQQKPGQAPRLLIYDASTR | 163 |
| | ATGIPARFSGSGSGTEFTLTISSLQSEDFAVYYCQQYNAWPYTFGQGTKVEIK | |
| 9-E4_VH | EVQLLESGGGLVQPGGSLRLSCAASGFTFSSYAMSWVRQAPGKGLEWVSVISGS | 164 |
| | GGNTYYADSVKGRFTISRDNSKNTLYLQMNSLRAEDTAVYYCAREVRHDSYYYY | |
| | YYSGMDVWGQGTLVTVSS | |
| 9-E4_VL | EIVMTQSPATLSVSPGERATLSCRASQSVYNNLAWYQQKPGQAPRLLIYDASTR | 165 |
| | ATGIPARFSGSGSGTEFTLTISSLQSEDFAVYYCQQYSDYPFTFGQGTKVEIK | |
| 9-E5_VH | QVQLVESGGGLVKPGGSLRLSCAASGFTFSNAWMSWVRQAPGKGLEWVGRIKSK | 166 |
| | IEGGTTDYAPPVKGRFTISRDDSKNTLYLQMNSLKTEDTAVYYCARDPTWYVSG | |
| | HYYGFDVWGQGTLVTVSS | |
| 9-E5_VL | DIQMTQSPSSLSASVGDRVTITCRASQSIANYLNWYQQKPGKAPKLLIYAASSL | 167 |
| | QSGVPSRFSGSGSGTDFTLTISSLQPEDFATYYCQQYFSTPPTFGQGTKVEIK | |
| 9-G1_VH | EVQLLESGGGLVQPGGSLRLSCAASGFTFGNYAMSWVRQAPGKGLEWVSTISAG | 168 |
| | GSRTYYADSVKGRFTISRDNSKNTLYLQMNSLRAEDTAVYYCARGWYPILYYAF | |
| | DYWGQGTLVTVSS | |
| 9-G1_VL | DIQMTQSPSSLSASVGDRVTITCRASQSIGNYLNWYQQKPGKAPKLLIYSASSL | 169 |
| | QSGVPSRFSGSGSGTDFTLTISSLQPEDFATYYCQQTYTIPLTFGQGTKVEIK | |
| 9-G3_VH | EVQLLESGGGLVQPGGSLRLSCAASGFTFSSYAMSWVRQAPGKGLEWVSTIYRS | 170 |
| | GGRTYYADSVKGRFTISRDNSKNTLYLQMNSLRAEDTAVYYCARGAYPLGSYYG | |
| | FDPWGQGTLVTVSS | |
| 9-G3_VL | DIQMTQSPSSLSASVGDRVTITCRASQSIATYLNWYQQKPGKAPKLLIYAASSL | 171 |
| | QSGVPSRFSGSGSGTDFTLTISSLQPEDFATYYCQQSYTYPVTFGQGTKVEIK | |
| 9-H9_VH | EVQLLESGGGLVQPGGSLRLSCAASGFTFSNYAMSWVRQAPGKGLEWVSTIYGS | 172 |
| | GVRTYYADGVKGRFTISRDNSKNTLYLQMNSLRAEDTAVYYCARGWIPHSFYAF | |
| | DYWGQGTLVTVSS | |
| 9-H9_VL | DIQMTQSPSSLSASVGDRVTITCRASQSISSYLNWYQQKPGKAPKLLIYGASSL | 173 |
| | QSGVPSRFSGSGSGTDFTLTISSLQPEDFATYYCQQSFTAPVTFGQGTKVEIK | |
| 2-C8_VH | EVQLLESGGGLVQPGGSLRLSCAASGFTFTNYAMSWVRQAPGKGLEWVSSISGS | 174 |
| | GISTYYADSVKGRFTISRDNSKNTLYLQMNSLRAEDTAVYYCARGPGPGYWYAF | |
| | DVWGQGTLVTVSS | |
| 2-C8_VL | DIQMTQSPSSLSASVGDRVTITCRASQTISRYLNWYQQKPGKAPKLLIYAASSL | 175 |
| | QSGVPSRFSGSGSGTDFTLTISSLQPEDFATYYCQQSYTYPITFGQGTKVEIK | |
| 8-D9_VH | QVQLVQSGAEVKKPGSSVKVSCKASGGTFSDEAIHWVRQAPGQGLEWMGRIIPV | 176 |
| | LGIASYAQKFQGRVTITADESTSTAYMELSSLRSEDTAVYYCARGLSRRAFYFD | |
| | YWGQGTLVTVSS | |
| 8-D9_VL | DIQMTQSPSSLSASVGDRVTITCRASQTIGNYLNWYQQKPGKAPKLLIYVASSL | 177 |
| | QSGVPSRFSGSGSGTDFTLTISSLQPEDFATYYCQQSYRYPYTFGQGTKVEIK | |
| 10- | EVQLLESGGGLVQPGGSLRLSCAASGFTFTNYAMSWVRQAPGKGLEWVSSISGS | 178 |
| A9_VH | GISTYYADSVKGRFTISRDNSKNTLYLQMNSLRAEDTAVYYCARGPGPGYWYAF | |
| | DVWGQGTLVTVSS | |
| 10- | DIQMTQSPSSLSASVGDRVTITCRASQTISRYLNWYQQKPGKAPKLLIYSASSL | 179 |
| A9_VL | QSGVPSRFSGSGSGTDFTLTISSLQPEDFATYYCQQSYTYPITFGQGTKVEIK | |
| 10- | EVQLLESGGGLVQPGGSLRLSCAASGFTFSDYAMSWVRQAPGKGLEWVSTIYSG | 180 |
| B2_VH | GVRTYYADSVKGRFTISRDNSKNTLYLQMNSLRAEDTAVYYCARGLVSFKGYAF | |
| | DYWGQGTLVTVSS | |
| 10- | DIQMTQSPSSLSASVGDRVTITCRASQSISSYLNWYQQKPGKAPKLLIYAASSL | 181 |
| B2_VL | QSGVPSRFSGSGSGTDFTLTISSLQPEDFATYYCQQSYTIPPTFGQGTKVEIK | |
| 10- | EVQLLESGGGLVQPGGSLRLSCAASGFTFSSYAMSWVRQAPGKGLEWVSAIYGA | 182 |
| B5_VH | GGKTYYADSVKGRFTISRDNSKNTLYLQMNSLRAEDTAVYYCARISHGWGFDVW | |
| | GQGTLVTVSS | |
| 10- | DIQMTQSPSSLSASVGDRVTITCRASQDISNYLNWYQQKPGKAPKLLIYAASSL | 183 |
| B5_VL | QSGVPSRFSGSGSGTDFTLTISSLQPEDFATYYCQQSYTYPLTFGQGTKVEIK | |
| 10- | EVQLLESGGGLVQPGGSLRLSCAASGFTFSNYAMSWVRQAPGKGLEWVSVIYGS | 184 |
| C1_VH | GTRTYYADSVKGRFTISRDNSKNTLYLQMNSLRAEDTAVYYCARVASSYHYAFD | |
| | YWGQGTLVTVSS | |
| 10- | DIQMTQSPSSLSASVGDRVTITCRASQSISTYLNWYQQKPGKAPKLLIYAASSL | 185 |
| C1_VL | QSGVSSRFSGSGSGTDFTLTISSLQPEDFATYYCQQSYTFPLTFGQGTKVEIK | |
| 10- | EVQLLESGGGLVQPGGSLRLSCAASGFTFSNYAMSWVRQAPGKGLEWVSVIYGS | 186 |
| C3_VH | GGRTYYADSVKGRFTISRDNSKNTLYLQMNSLRAEDTAVYYCARVEDTSYYGMD | |
| | YWGQGTLVTVSS | |
| 10- | DIQMTQSPSSLSASVGDRVTITCRASQTIGIYLNWYQQKPGKAPTLLIYAASSL | 187 |
| C3_VL | QSGVPSRFSGSGSGTDFTLTISSLQPEDFATYYCQQSYTYPLTFGQGTKVEIK | |
| 10- | EVQLLESGGGLVQPGGSLRLSCAASGFTFSNYAMSWVRQAPGKGLEWVSAIYGS | 188 |
| C9_VH | GGRTYYADSVKGRFTISRDNSKNTLYLQMNSLRAEDTAVYYCARLEAGFYSLDI | |
| | WGQGTLVTVSS | |
| 10- | DIQMTQSPSSLSASVGDRVTITCRASQTIGTYLNWYQQKPGKAPKLLIYAASSL | 189 |
| C9_VL | QSGVPSRFSGSGSGTDFTLTISSLQPEDFATYYCQQSYSYPLTFGQGTKVEIK | |
| 10- | EVQLLESGGGLVQPGGSLKLSCAASGFTFGSYAMSWVRQAPGKGLEWVSVISSG | 190 |
| C11_VH | GSYTYYADSVKGRFTISRDNSKNTLYLQMNSLRAEDTAVYYCARVSGVVGALDI | |
| | WGQGTLVTVSS | |
| 10- | EIVMTQSPATLSVSPGERATLSCRASQSVSNNLAWYQQKPGQAPRLLIYGASTR | 191 |
| C11_VL | ATGIPARFSGSGSGTEFTLTISSLQSEDFAVYYCQQYNNWPYTFGQGTKVEIK | |
| 10- | EVQLLESGGGLVQPGGSLRLSCAASGFTFSNYAMSWVRQAPGKGLEWVSTIYSS | 192 |
| D7_VH | GSRTYYADSVKGRFTISRDNSKNTLYLQMNSLRAEDTAVYYCARWGGDGFYAMD | |
| | YWGQGTLVTVSS | |
| 10- | DIQMTQSPSSLSASVGDRVTITCRASQSISTYLNWYQQKPGKAPKLLIYAASSL | 193 |
| D7_VL | QSGVPSRFSGSGSGTDFTLTISSLQPEDFATYYCQQSYTVPLTFGQGTKVEIK | |
| 10- | EVQLLESGGGLVQPGGSLRLSCAASGFTFSDYAMSWVRQAPGKGLEWVSTIYSG | 194 |
| E1_VH | GVRTYYADSVKGRFTISRDNSKNTLYLQMNSLRAEDTAVYYCARGLVSFKGYAF | |
| | DYWGQGTLVTVSS | |
| 10- | DIQMTQSPSSLSASVGDRVTITCRASQSISSYLNWYQQKPGKAPKLLIYAASSL | 195 |
| E1_VL | QSGVPSRFSGSGSGTDFTLTISSLQPEDFATYYCQQSYTIPPTFGQGTKVEIK | |
| 10- | EVQLLESGGGLVQPGGSLRLSCAASGFTFSSYAMSWVRQAPGKGLEWVSAIYGS | 196 |
| E6_VH | GGDTYYADSVKGRFTISRDNSKNTLYLQMNSLRAEDTAVYYCARTEYDGYFDVW | |
| | GQGTLVTVSS | |
| 10- | DIQMTQSPSSLSASVGDRVTITCRASQDIYNYLNWYQQKPGKAPKLLIYGASSL | 197 |
| E6_VL | QSGVPSRFSGSGSGTDFTLTISSLQPEDFATYYCQQSDGFPPTFGQGTKVEIK | |
| 10- | EVQLLESGGGLVQPGGSLRLSCAASGFTFTDYAMSWVRQAPGKGLEWVSVIYGS | 198 |
| E7_VH | GGRTYYADSVKGRFTISRDNSKNTLYLQMNSLRAEDTAVYYCARVEDSHYSFDV | |
| | WGQGTLVTVSS | |
| 10- | DIQMTQSPSSLSASVGDRVTITCRASQSISNYLNWYQQKPGKAPKLLIYAASSL | 199 |
| E7_VL | QSGVPSRFSGSGSGTDFTLTISSLQPEDFATYYCQQSYTVPWTFGQGTKVEIK | |
| 10- | QVQLVESGGGVVQPGRSLRLSCAASGFPFSDYAMHWLRQAPGKGLEWVAVISYD | 200 |
| F11_VH | GNIEYYANSVKGRFTISRDNSKNTLYLQMNSLRAEDTAVYYCARDRIGSSTYYG | |
| | YYNGFDVWGQGTLVTVSS | |
| 10- | EIVMTQSPATLSVSPGERATLSCRASQSVSSNLAWYQQKPGQAPRLLIYGASTR | 201 |
| F11_VL | ATGIPARFSGSGSGTEFTLTISSLQSEDFAVYYCQQYNDYPLTFGQGTKVEIK | |
| 10- | EVQLLESGGGLVQPGGSLRLSCAASGFTFSNYAMSWVRQAPGKGLEWVSAIKGS | 202 |
| F12_VH | GGGTYYADSVKGRFTISRDNSKNTLYLQMNSLRAEDTAVYYCARGGYLWLDPWG | |
| | QGTLVTVSS | |
| 10- | EIVMTQSPATLSVSPGERATLSCRASQSISSNLAWYQQKPGQAPRLLIYDASTR | 203 |
| F12_VL | ATGIPARFSGSGSGTEFTLTISSLQSEDFAVYYCQQYYNWPLTFGQGTKVEIK | |
| 10- | EVQLLESGGGLVQPGGSLRLSCAASGFTFSNYAMSWVRQAPGKGLEWVSAISGS | 204 |
| G5_VH | GTYTYYADSVKGRFTISRDNSKNTLYLQMNSLRAEDTAVYYCARVRISGYGPGY | |
| | FGGFDVWGQGTLVTVSS | |
| 10- | EIVMTQSPATLSVSPGERATLSCRASQSVYSNLAWYQQKPGQAPRLLIYGASTR | 205 |
| G5_VL | ATGIPARFSGSGSGTEFTLTISSLQSEDFAVYYCQQYSNWPLTFGQGTKVEIK | |
| 11- | EVQLLESGGGLVQPGGSLRLSCAASGFTFSRYAMSWVRQAPGKGLEWVSIISGT | 206 |
| B6_VH | GGNTYYADSVKGRFTISRDNSKNTLYLQMNSLRAEDTAVYYCARIDTFAYYYSL | |
| | YRAFDLWGQGTLVTVSS | |
| 11- | EIVMTQSPATLSVSPGERATLSCRASQSVGGNLAWYQQKPGQAPRLLIYDASTR | 207 |
| B6_VL | ATGIPARFSGSGSGTEFTLTISSLQSEDFAVYYCQQYNDWPLTFGQGTKVEIK | |
| 11- | EVQLLESGGGLVQPGGSLRLSCAASGFTFSDYAMSWVRQAPGKGLEWVSSISEG | 208 |
| D8_VH | GGSTYYADSVKGRFTISRDNSKNTLYLQMNSLRAEDTAVYYCARTKRYFAGGSY | |
| | YWMDVWGQGTLVTVSS | |
| 11- | DIQMTQSPSSLSASVGDRVTITCRASQRISSYLNWYQQKPGKAPKLLIYAASSL | 209 |
| D8_VL | QSGVPSRFSGSGSGTDFALTISSLQPEDFATYYCQQSYSYPITFGQGTKVEIK | |
| 11- | EVQLLESGGGLVQPGGSLRLSCAASGFTFSSYAMSWVRQAPGKGLEWVSVISGG | 210 |
| D10_VH | SGHTTYYADSVKGRFTISRDNSKNTLYLQMNSLRAEDTAVYYCARHGGGRFAMD | |
| | VWGQGTLVTVSS | |
| 11- | EIVLTQSPGTLSLSPGERATLSCRASQSVASPYLAWYQQKPGQAPRLLIYGASS | 211 |
| D10_VL | RATGIPDRFSGSGSGTDFTLTISRLEPEDFAVYYCQQYYESPITFGQGTKVEIK | |
| 11- | EVQLLESGGGLVQPGGSLRLSCAASGFTFSTYAMSWVRQAPGKGLEWVSSISGS | 212 |
| D11_VH | GGKTYYADSVKGRFTISRDNSKNTLYLQMNSLRAEDTAVYYCARDIYGITYDGY | |
| | TYYDGMDVWGQGTLVTVSS | |
| 11- | DIQMTQSPSSLSASVGDRVTITCRASQSIYNYLNWHQQKPGKAPKLLIYAASSL | 213 |
| D11_VL | QSGVPSRFSGSGSGTDFTLTISSLQPEDFATYYCQQSDSPPWTFGQGTKVEIK | |
| 11- | QVQLVQSGAEVKKPGSSVKVSCKASGGTFSNYYISWVRQAPGQGLEWMGGIIPV | 214 |
| G2_VH | FGVAHYAQKFQGRVTITADESTSTAYMELSSLRSEDTAVYYCARGYPGEYFDYW | |
| | GQGTLVTVSS | |
| 11- | EIVLTQSPGTLSLSPGERATLSCRASQSVSSSYLAWYQQKPGQAPRLLIYAASS | 215 |
| G2_VL | RATGIPDRFSGSGSGTDFTLTISRLEPEDFAVYYCQHYGGSPGYTFGQGTKVEI | |
| | K | |
| 11- | EVQLLESGGGLVQPGGSLRLSCAASGFTFSNYAMSWVRQAPGKGLEWVSTISGS | 216 |
| G5_VH | GANTYYADSVKGRFTISRDNSKNTLYLQMNSLRAEDTAVYYCARHHDIAPSWIY | |
| | YFDYWGQGTLVTVSS | |
| 11- | EIVLTQSPGTLSLSPGERATLSCRARQSVPSNYLAWYQQKPGQAPRLLIYGASS | 217 |
| G5_VL | RATGIPDRFSGSGSGTDFTLTISRLEPEDFAVYYCQQYYSSPLTFGQGTKVEIK | |
| 11- | EVQLLESGGGLVQPGGSLRLSCAASGFTFPDYAMSWVRQAPGKGLEWVSTIYAG | 218 |
| G9_VH | GGRTYYADSVKGRFTISRDNSKNTLYLQMNSLRAEDTAVYYCARGLTRHYGYSF | |
| | DHWGQGTLVTVSS | |
| 11- | DIQMTQSPSSLSASVGDRVTITCRASQTISSYLNWYQQKPGKAPKLLIYSASSL | 219 |
| G9_VL | QSGVPSRFSGSGSGTDFTLTISSLQPEDFATYYCQQSYTFPLTFGQGTKVEIK | |
| 11- | EVQLLESGGGLVQPGGSLRLSCAASGFTFSNYAMSWVRQAPGKGLEWVSSIYGS | 220 |
| H8_VH | GHRTYYADSVKGRFTISRDNSKNTLYLQMNSLRAEDTAVYYCARVASSAEYAFD | |
| | HWGQGTLVTVSS | |
| 11- | DIQMTQSPSSLSASVGDRVTITCRASQSISNYLNWYQQKSGKAPKLLIYAASSL | 221 |
| H8_VL | QSGVPSRFSGSGSGTDFTLTISSLQPEDFATYYCQQSYTVPLTFGQGTKVEIK | |
| 11- | EVQLLESGGGLVQPGGSLRLSCAASGFTFKNYAMSWVRQAPGKGLEWVSGISEG | 222 |
| H11_VH | GANTYYADSVKGRFTISRDNSKNTLYLQMNSLRAEDTAVYYCAREPPKYGAYSY | |
| | YGYGFDPWGQGTLVTVSS | |
| 11- | EIVLTQSPGTLSLSPGERATLSCRASQSVSSSYLAWYQQKPGQAPRLLIYGASS | 223 |
| H11_VL | RATGIPDRFSGSGSGTDFTLTISRLEPEDFAVYYCQQYSSSPTYTFGQGTKVEI | |
| | K | |

TABLE 7
CDR Sequences of the Antibodies (Kabat)

| | | SEQ | | SEQ | | SEQ |
|---|---|---|---|---|---|---|
| | | ID | | ID | | ID |
| Name | CDR1 | NO: | CDR2 | NO: | CDR3 | NO: |
| 1-A1_VH | SYAMS | 224 | AISAGGGYTYYADSVKG | 248 | YDYNSYFDS | 341 |
| 1-A5_VH | SYGMH | 225 | VISHSGSNKYYANSVKG | 249 | GLAGWEVYPFDV | 342 |
| 1-H10_VH | NYAMS | 226 | TISGGGTYYADSVKG | 250 | SRYWYTKLISYTYGMDV | 343 |
| 2-B4_VH | NYGMH | 227 | VISYHGRNEYYADSVKG | 251 | GDDQDAFDP | 344 |
| 2-B5_VH | DYDMH | 228 | VISHSGSNKYYADSVKG | 252 | DRSVYLYYGYHYYEGFDV | 345 |
| 2-F7_VH | DYAMS | 229 | SIYTSGGRTYYADSVKG | 253 | GYRYFDH | 346 |
| 2-F8_VH | NYAMS | 226 | TIYGRGERTYYADSVKG | 254 | VSVVWSYAFDY | 347 |
| 2-G4_VH | SSNYYWG | 230 | SVYYTGRTYYNPSLKS | 255 | VFPYGAVDV | 348 |
| 2-H2_VH | DYGMH | 231 | VISYEGSNEYYADSVKG | 256 | GSEGSGFDV | 349 |
| 2-H9_VH | TYAMS | 232 | SIYGSGGKTYYADSVKG | 257 | TDYSLYYQFDY | 350 |
| 3-A7_VH | NYGMH | 227 | VISFDGSNKYYANSVKG | 258 | GGDEDGFDV | 351 |
| 3-A9_VH | NYAMS | 226 | SITGSGEYTNYADSVKG | 259 | EVADYWDGGVYYYYDGGF | 352 |
| | | | | | DV | |
| 3-B3_VH | SYAMS | 224 | TISGTGGRTYYADSVKG | 260 | VDHYSGYAFDL | 353 |
| 3-C4_VH | SYAMS | 224 | TIYGGGSYTYYADSVKG | 261 | AEVDDYWYLYMDV | 354 |
| 3-C6_VH | TYAMS | 232 | TIYGSGGRTYYADSVKG | 262 | TDHYSYYVMDY | 355 |
| 3-C8_VH | DYAMS | 229 | AIYGSGSYTYYADSVKG | 263 | TAYSYPYVYFDF | 356 |
| 3-C9_VH | NYAMS | 226 | VISGGGTNTYYADSVKG | 264 | SDSYYGVYDGYYYGMDV | 357 |
| 3-C11_VH | SYAMS | 224 | TIYASGATTYYADSVKG | 265 | LKLYHAAFDI | 358 |
| 3-C12_VH | DYAMS | 229 | TIYGSGSRTYYADSVKG | 266 | VDHVHGYAFDY | 359 |
| 3-D3_VH | NYAMS | 226 | TIYESGGYTYYADSVKG | 267 | VESYGYGEYITYNYYGFD | 360 |
| | | | | | V | |
| 3-D6_VH | SYGIH | 233 | RIVPILGTTNYAQKFQG | 268 | DPAVTLDTYVYADHGFDV | 361 |
| 3-D7_VH | NYAMS | 226 | GISEGGANTYYADSVKG | 269 | EPPKYGAYSYYGYGFDP | 362 |
| 3-D12_VH | GYAMS | 234 | SISVSGAGTYYADSVKG | 270 | YVRGVSFDV | 363 |
| 3-E2_VH | NYAMS | 226 | AIGGSGGGTYYADSVKG | 271 | YGWEYQAFDY | 364 |
| 3-E5_VH | NYAMS | 226 | GITAGGGNTYYADSVKG | 272 | IAGLFDGFDV | 365 |
| 3-E7_VH | NYAMS | 226 | GISASGGGTYYADSVKG | 273 | SSRYGDTFDV | 366 |
| 3-E8_VH | DYAMS | 229 | GISVSGGYTYYADSVKG | 274 | GLYAHHTYYYGGFDY | 367 |
| 3-E9_VH | SYAMS | 224 | TIYGTGEDTYYADSVKG | 275 | DYFWSPRGSIYDYDYFDV | 368 |
| 3-F5_VH | GYAMS | 234 | SISVSGAGTYYADSVKG | 270 | YVRGVSFDV | 363 |
| 3-F6_VH | DYAMS | 229 | AISSSGGTTYYADSVKG | 276 | DAGWSYYGLWIYYYYMDV | 369 |
| 3-F12_VH | DYAMS | 229 | GISGSGGYTYYADSVKG | 277 | KLGGFDENYTYGMDV | 370 |
| 3-G4_VH | TYAMS | 232 | GISAGGGARTYYADSVKG | 278 | VHHFGYYSLDV | 371 |
| 3-G5_VH | SYAMS | 224 | VIYGSGGRTYYADSVKG | 279 | HDPRYQLDV | 372 |
| 3-G6_VH | DYGMH | 231 | VISYDGSRKYYANSVKG | 280 | GSYSDGFDV | 373 |
| 3-G7_VH | TEYIS | 235 | RIIPVLGITNYAQKFQG | 281 | HLGPPYLVTYSHGFDV | 374 |
| 3-G8_VH | NYAMS | 226 | AIYGSGGKTYYADSVKG | 282 | LKYGFYQFDY | 375 |
| 3-H1_VH | NYAMS | 226 | VIYAGGARTYYADSVKG | 283 | WGGDGFYAMDY | 376 |
| 3-H4_VH | NYAMS | 226 | AISGSGGTTYYADSVKG | 284 | IKKYGSAGYTVGFDS | 377 |
| 4-A7_VH | SSSYYWG | 236 | NIYYTGTTYYNPSLKS | 285 | IKVPWYYSSYYFDY | 378 |
| 4-A8_VH | NYAMS | 226 | AISASGGYTYYADSVKG | 286 | GRWGAFDY | 379 |
| 4-B1_VH | DYAMS | 229 | GISRGGARTYYADSVKG | 287 | YLFGHYMD | 380 |
| 4-B2_VH | RYAMH | 237 | VISHSGSTKYYANSVKG | 288 | SRGWRGFDS | 381 |
| 4-B11_VH | GYGMH | 238 | VISHHGSYKYYANSVKG | 289 | GGADVGYFYSTFYYYMDV | 382 |
| 4-D12_VH | NYAMS | 226 | AISRGGGYTYYADSVKG | 290 | WGGDGFYAMDY | 376 |
| 4-F4_VH | SRGYYWA | 239 | SIYYSGSTYYNPSLKS | 291 | LVSYGAYYVFDY | 383 |
| 4-G12_VH | SYAMS | 224 | AISGSGGSTYYADSVKG | 292 | DKRLTY | 384 |
| 5-C9_VH | SYAIN | 240 | RIIPLLETADYAQKFQG | 293 | EWPGEYFDV | 385 |
| 5-E2_VH | DYGMH | 231 | VISYHGRNEYYANSVKG | 294 | GAYGDDFDV | 386 |
| 5-E12_VH | NYAMS | 226 | TISGGGRTTNYADSVKG | 293 | STPIPSYYPYIYSYYFDV | 387 |
| 6-G4_VH | DYAMS | 229 | SISSSGANTYYADSGKG | 296 | SVVTWVTYAFDY | 388 |
| 6-H5_VH | NYAMS | 226 | AISATGGATYYADSVKG | 297 | SVRERYTYYMDY | 389 |
| 8-A2_VH | NYYIS | 241 | GIIPVFGVAHYAQKFQG | 298 | GYPGEYFDY | 390 |
| 8-A4_VH | SYAMS | 224 | SIYAGGGRTYYADSVKG | 299 | WGGDGFYAMDY | 376 |
| 8-A6_VH | DYAMS | 229 | TIYSGGVRTYYADSVKG | 300 | GLVSFRGYAFDY | 391 |
| 8-A7_VH | NYAMS | 226 | SISGGGINTYYADSVKG | 301 | VRSYSGPGYFDY | 392 |
| 8-A8_VH | NYAMS | 226 | VIYGSGARTYYADSVKG | 302 | ISPHYDFDV | 393 |
| 8-A9_VH | NYAMS | 226 | AISGGGSRTYYADSVKG | 303 | EARVLGYSLDY | 394 |
| 8-C4_VH | NYAMS | 226 | AIYGSGGRTYYADSVKG | 304 | LRYGFYQFDY | 395 |
| 8-C6_VH | DYAMS | 229 | TIYSSGHRTYYADSVKG | 305 | LYPLHSYAFDH | 396 |
| 8-C9_VH | SYAMS | 224 | LISSSGENTYYADSVKG | 306 | VRFGYGSWRYRKYMDV | 397 |
| 8-C10_VH | SYAMS | 224 | TISASGATYYADSVKG | 307 | GPYSYYYYSFDY | 398 |
| 8-C11_VH | SYAMS | 224 | TIYVGGHRTYYADSVKG | 308 | VGWSWGFDP | 399 |
| 8-D11_VH | NAWMS | 242 | RIKSITEGGTTDYAAPVKG | 309 | VWHTDDYYEGFDV | 400 |
| 8-04_VH | SYAMS | 224 | TIYGSGARTYYADSVKG | 310 | WGGDGFYAMDY | 376 |
| 8-07_VH | SYAMS | 224 | GISTSGGTTYYADSVKG | 311 | VVGRAVFDI | 401 |
| 8-E2_VH | DEAIH | 243 | RIIPVLGIASYAQKFQG | 312 | GLSRKAFYFDY | 402 |
| 8-E3_VH | NYAMS | 226 | TIYRGAGTYYADSVKG | 313 | DASWYGPFSYYYYYGFDV | 403 |
| 8-E9_VH | DYAMS | 229 | IITESGVNTYYADSVKG | 314 | HPSVGSASRSYYFDV | 404 |
| 8-F1_VH | DYAMS | 229 | GISGSGGVGTYYADSVKG | 313 | GEYGSSIAYSYYYGFDV | 405 |
| 8-F2_VH | NYAMS | 226 | AISGGGSRTYYADSVKG | 303 | EARVLGYSLDY | 394 |
| 8-F4_VH | GYAMS | 234 | AIRGGGGTYYADSVKG | 316 | VGPLRLYGFDY | 406 |
| 8-F6_VH | SYAMS | 224 | VISGSGGNTYYADSVKG | 317 | EVRHDSYYYYYYSGMDV | 407 |
| 8-F11_VH | NYAIN | 244 | RIIPTLGIANYAQKFQG | 318 | AAYFAGPAARYGYFDI | 408 |
| 8-G12_VH | GYAMS | 234 | SISVSGAGTYYADSVKG | 270 | YVRGVSFDV | 363 |
| 8-H7_VH | NYAMS | 226 | AIYGSGGRTYYADSVKG | 304 | LRYGFYQFDY | 395 |
| 9-A6_VH | DYAMS | 229 | TIYSGGVRTYYADSVKG | 300 | GLVSFRGYAFDY | 391 |
| 9-B3_VH | DYGMH | 231 | VISYDGSRKYYANSVKG | 280 | GSYSDGFDV | 373 |
| 9-B10_VH | SYAMS | 224 | GISESGGGTYYADSVKG | 319 | LVPWQLAFDV | 409 |
| 9-C2_VH | SYAMS | 224 | VISGSGGNTYYADSVKG | 317 | EVRHDSYYYYYYSGMDV | 407 |
| 9-D8_VH | SEAIS | 245 | RIIPISGRPNYAQKFQG | 320 | EASYYVAGYYRYYGFDV | 410 |
| 9-E4_VH | SYAMS | 224 | VISGSGGNTYYADSVKG | 317 | EVRHDSYYYYYYSGMDV | 407 |
| 9-E5_VH | NAWMS | 242 | RIKSKIEGGTTDYAPPVKG | 321 | DPTWYVSGHYYGFDV | 411 |
| 9-G1_VH | NYAMS | 226 | TISAGGSRTYYADSVKG | 322 | GWYPILYYAFDY | 412 |
| 9-G3_VH | SYAMS | 224 | TIYRSGGRTYYADSVKG | 323 | GAYPLGSYYGFDP | 413 |
| 9-H9_VH | NYAMS | 226 | TIYGSGVRTYYADGVKG | 324 | GWIPHSFYAFDY | 414 |
| 2-C8_VH | NYAMS | 226 | SISGSGISTYYADSVKG | 323 | GPGPGYWYAFDV | 415 |
| 8-D9_VH | DEAIH | 243 | RIIPVLGIASYAQKFQG | 312 | GLSRRAFYFDY | 416 |
| 10-A9_VH | NYAMS | 226 | SISGSGISTYYADSVKG | 325 | GPGPGYWYAFDV | 415 |
| 10-B2_VH | DYAMS | 229 | TIYSGGVRTYYADSVKG | 300 | GLVSFKGYAFDY | 417 |
| 10-B5_VH | SYAMS | 224 | AIYGAGGKTYYADSVKG | 326 | ISHGWGFDV | 418 |
| 10-C1_VH | NYAMS | 226 | VIYGSGTRTYYADSVKG | 327 | VASSYHYAFDY | 419 |
| 10-C3_VH | NYAMS | 226 | VIYGSGGRTYYADSVKG | 279 | VEDTSYYGMDY | 420 |
| 10-C9_VH | NYAMS | 226 | AIYGSGGRTYYADSVKG | 304 | LEAGFYSLDI | 421 |
| 10- | SYAMS | 224 | VISSGGSYTYYADSVKG | 328 | VSGVVGALDI | 422 |
| C11_VH | | | | | | |
| 10-D7_VH | NYAMS | 226 | TIYSSGSRTYYADSVKG | 329 | WGGDGFYAMDY | 376 |
| 10-E1_VH | DYAMS | 229 | TIYSGGVRTYYADSVKG | 300 | GLVSFKGYAFDY | 417 |
| 10-E6_VH | SYAMS | 224 | AIYGSGGDTYYADSVKG | 330 | TEYDGYFDV | 423 |
| 10-E7_VH | DYAMS | 229 | VIYGSGGRTYYADSVKG | 279 | VEDSHYSFDV | 424 |
| 10- | DYAMH | 246 | VISYDGMIEYYANSVKG | 331 | DRIGSSTYYGYYNGFDV | 425 |
| F11_VH | | | | | | |
| 10- | NYAMS | 226 | AIKGSGGGTYYADSVKG | 332 | GGYLWLDP | 426 |
| F12_VH | | | | | | |
| 10-G5_VH | NYAMS | 226 | AISGSGTYTYYADSVKG | 333 | VRISGYGPGYFGGFDV | 427 |
| 11-B6_VH | RYAMS | 247 | IISGTGGNTYYADSVKG | 334 | IDTFAYYYSLYRAFDL | 428 |
| 11-D8_VH | DYAMS | 229 | SISEGGGSTYYADSVKG | 335 | TKRYFAGGSYYWMDV | 429 |
| 11- | SYAMS | 224 | VISGGSGHTTYYADSVKG | 336 | HGGGRFAMDV | 430 |
| D10_VH | | | | | | |
| 11- | TYAMS | 232 | SISGSGGKTYYADSVKG | 337 | DIYGITYDGYTYYDGMDV | 431 |
| D11_VH | | | | | | |
| 11-G2_VH | MYYIS | 241 | GIIPVFGVAHYAQKFQG | 298 | GYPGEYFDY | 390 |
| 11-G5_VH | NYAMS | 226 | TISGSGANTYYADSVKG | 338 | HHDIAPSWIYYFDY | 432 |
| 11-G9_VH | DYAMS | 229 | TIYAGGGRTYYADSVKG | 339 | GLTRHYGYSFDH | 433 |
| 11-H8_VH | NYAMS | 226 | SIYGSGHRTYYADSVKG | 340 | VASSAEYAFDH | 434 |
| 11- | NYAMS | 226 | GISEGGANTYYADSVKG | 269 | EPPKYGAYSYYGYGFDP | 362 |
| H11_VH | | | | | | |
| | | SEQ | | SEQ | | SEQ |
| | | ID | | ID | | ID |
| Name | CDR1 | NO: | CDR2 | NO: | CDR3 | NO: |
| 1-A1_VL | TGSSSMIGAGYDVH | 435 | GNNNRPS | 497 | QSYDRSLSGWV | 517 |
| 1-A5_VL | TGSSSMIGAGFDVH | 436 | DNTNRPS | 498 | QSYDYSLRVWV | 518 |
| 1-H10_VL | TGSSSMIGATYDVH | 437 | VNNNRPS | 499 | QSYDSSLRAWV | 519 |
| 2-B4_VL | RASQSISNYLN | 438 | AASSLQS | 500 | QQSFSAPYT | 520 |
| 2-B5_VL | RASQSVYNNLA | 439 | GASTRAT | 501 | QQYNDLPLT | 521 |
| 2-F7_VL | RASQSVRNNLA | 440 | GASTRAT | 501 | QQYYTWPRT | 522 |
| 2-F8_VL | RASQDISTYLN | 441 | AASSLQS | 500 | QQSYSYPLT | 523 |
| 2-G4_VL | RASQSVSGSYLA | 442 | GASSRAT | 502 | QQYGGSLPYT | 524 |
| 2-H2_VL | RASQRISNYLN | 443 | AASSLQS | 500 | QQSYSPPYT | 525 |
| 2-H9_VL | RASQSISRYLN | 444 | AASSLQS | 500 | QQSYSYPVT | 526 |
| 3-A7_VL | RASQSISSYLN | 445 | AASSLQS | 300 | QQSYRPPYT | 527 |
| 3-A9_VL | RASQSVSSNYLA | 446 | GASSRAT | 502 | QQYGRSAPIT | 528 |
| 3-B3_VL | RASQSISSYLN | 445 | AASSLQS | 500 | QQSYSYPLT | 523 |
| 3-C4_VL | RASQDIGNYLN | 447 | AASSLQS | 300 | QQSYTFPLT | 529 |
| 3-C6_VL | RASQSISKYLN | 448 | SASSLQS | 503 | QQSYSFPLT | 530 |
| 3-C8_VL | RASQTITSYLN | 449 | AASSLQS | 300 | QQSYTYPLT | 531 |
| 3-C9_VL | RASQTISRYLN | 450 | AASSLQS | 300 | QQTYSTPWT | 532 |
| 3-C11_VL | RASQDISSYLN | 451 | TASSLQS | 504 | QQSYSYPLT | 523 |
| 3-C12_VL | RASQSISKYLN | 448 | AASSLQS | 300 | QQSYSFPPT | 533 |
| 3-D3_VL | RASQSVATGYLA | 452 | GASSRAT | 502 | QQYESSPPFT | 534 |
| 3-D6_VL | RASQSVYNNLA | 439 | GASTRAT | 501 | QQYNDWPLT | 535 |
| 3-D7_VL | RASQSVGSNYLA | 433 | GASSRAT | 502 | QQYGSSPYT | 536 |
| 3-D12_VL | RASQSVSSSYLA | 454 | GASSRAT | 302 | QQYETSPPVT | 537 |
| 3-E2_VL | RASQSISRYLN | 444 | AASSLQS | 300 | QQSYSTPWT | 538 |
| 3-E5_VL | RASQSVRSSYLA | 433 | GASSRAT | 502 | QQYASLPLT | 539 |
| 3-E7_VL | RASLSVGSNLA | 456 | GASTRAT | 501 | QQYNNLPLT | 540 |
| 3-E8_VL | RASQSVGSNLA | 437 | GASTRAT | 501 | QQYHNWPPT | 541 |
| 3-E9_VL | RASQSISRYLN | 444 | SASSLQS | 303 | QQYFSAPYT | 542 |
| 3-F5_VL | RASQSVSSNYLA | 446 | GASSRAT | 502 | QQYGSSPAVT | 543 |
| 3-F6_VL | RASQSVRSNYLA | 438 | GASSRAT | 502 | QQYGASPQT | 544 |
| 3-F12_VL | RASQSVSSSYLA | 454 | GASSRAT | 502 | QQYGSSPEYT | 545 |
| 3-G4_VL | RASQTISNYLN | 439 | AASSLQS | 300 | QQSYTYPLT | 531 |
| 3-G5_VL | RASQSIGSYLN | 460 | TASSLQS | 504 | QQSYTYPLT | 531 |
| 3-G6_VL | RASQSISRYLN | 444 | GASSLQS | 303 | QQSYSTPYT | 546 |
| 3-G7_VL | RASQSLGTNLA | 461 | GASTRAT | 501 | QQYYEAPPT | 547 |
| 3-G8_VL | RASQSIRNYLN | 462 | AASSLQS | 300 | QQSYSYPLT | 523 |
| 3-H1_VL | RASQTISTYLN | 463 | AASILQS | 506 | QQSYTVPLT | 548 |
| 3-H4_VL | RASQSVGSYLA | 464 | GASTRAT | 501 | QQYYNAPIT | 549 |
| 4-A7_VL | TGSSSNIGAGYDVH | 433 | GNKNRPS | 307 | QSYDTSLSAWV | 550 |
| 4-A8_VL | TGSSSNIGSGYDVH | 465 | GNRNRPS | 308 | QSYDSSLKTWV | 551 |
| 4-B1_VL | TGSSSNIGAGYDVH | 433 | GNTNRPS | 309 | QSYDGRLGVSV | 552 |
| 4-B2_VL | TGSSSNIGAYVVH | 466 | GNTNRPS | 309 | QSYDRTLSSYV | 553 |
| 4-B11_VL | TGSSSNIGAAFDVH | 467 | DNYNRPS | 510 | QSYDSSLSVWV | 554 |
| 4-D12_VL | TGSSSNIGAGYDVH | 433 | ANNNRPS | 511 | QSYDYSLSGWV | 555 |
| 4-F4_VL | TGSSSNIGAGYDVH | 433 | GNRNRPS | 308 | QSYDTRLSAWV | 556 |
| 4-G12_VL | SGSSSNIGNNYVS | 468 | DNNKRPS | 512 | GTWDSSLSARV | 557 |
| 5-C9_VL | RASQSVASSYLA | 469 | GASSRAT | 502 | QQYGSSQLT | 558 |
| 5-E2_VL | RASQSISSYLN | 445 | AASSLQS | 300 | QQSYSAPYT | 559 |
| 5-E12_VL | RASQSVSNNLA | 470 | GASTRAT | 501 | QQYGNYPPT | 560 |
| 6-G4_VL | TGSSSNIGAGYDVH | 433 | DNTNRPS | 498 | QSYDSGLSGWV | 561 |
| 6-H5_VL | TGSSSNIGAGYDVH | 433 | DNTNRPS | 498 | QSYDVSLGVWV | 562 |
| 8-A2_VL | RASQSVSSSYLA | 454 | AASSRAT | 313 | QHYGGSPGYT | 563 |
| 8-A4_VL | RASQTISNYLN | 439 | GASSLQS | 303 | QQSFTFPLT | 564 |
| 8-A6_VL | RASQSISSYLN | 445 | AASSLQS | 300 | QQSYTIPPT | 565 |
| 8-A7_VL | RASRSVSSNLA | 471 | GASTRAT | 501 | QQYYNWPLT | 566 |
| 8-A8_VL | RASQTISKYLN | 472 | AASSLQS | 500 | QQSYSYPIT | 567 |
| 8-A9_VL | RASQRIGKYLN | 473 | AASSLQS | 300 | QQSYSYPLT | 523 |
| 8-C4_VL | RASQSIRNYLN | 462 | AASSLQS | 300 | QQSYSYPLT | 523 |
| 8-C6_VL | RASQSISRYLN | 444 | AASSLQS | 300 | QQSYSVPLT | 568 |
| 8-C9_VL | RASQSIATYLN | 474 | GASSLQS | 303 | QQSYSTPLT | 569 |
| 8-C10_VL | RASQTISTYLN | 463 | AASSLQS | 300 | QQSYRYPLT | 570 |
| 8-C11_VL | RASQTISKYLN | 472 | SASSLQS | 303 | QQSYTYPLT | 531 |
| 8-011_VL | RASQSVSGSYLA | 442 | DASSRAT | 514 | QQYGTTPQT | 571 |
| 8-D4_VL | RASQSIYTYLN | 473 | AASSLQS | 300 | QQSYSFPLT | 530 |
| 8-D7_VL | RASQSVSSTYLA | 476 | AASSRAT | 313 | QQYAALPIT | 572 |
| 8-E2_VL | RASQTIGNYLN | 477 | VASSLQS | 313 | QQSYRYPYT | 573 |
| 8-E3_VL | RASQSVYTNLA | 478 | DASTRAT | 516 | QQYNDWPLT | 535 |
| 8-E9_VL | RASQSVGSSYLA | 479 | AASSRAT | 313 | QQYYSAPLT | 574 |
| 8-F1_VL | RASQSVYNNLA | 439 | DASTRAT | 516 | QQYNDWPYT | 575 |
| 8-F2_VL | RASQRIGKYLN | 473 | AASSLQS | 300 | QQSYSYPLT | 523 |
| 8-F4_VL | RASQSISNYLN | 438 | AASSLQS | 300 | QQSYSYPLT | 523 |
| 8-F6_VL | RASQSVYNNLA | 439 | DASTRAT | 516 | QQYSDYPFT | 576 |
| 8-F11_VL | RASQSVRSNYLA | 438 | DASSRAT | 514 | QQYAYAPWT | 577 |
| 8-G12_VL | RASQSVSSNYLA | 446 | GASSRAT | 502 | QQYGSSPAVT | 543 |
| 8-H7_VL | RASQSIRNYLN | 462 | AASSLQS | 300 | QQSYSYPLT | 523 |
| 9-A6_VL | RASQSISSYLN | 445 | AASSLQS | 300 | QQSYTIPPT | 565 |
| 9-B3_VL | RASQSISNYLN | 438 | AASSLQS | 300 | QQSYAPPYT | 578 |
| 9-B10_VL | RASQSVYNNLA | 439 | DASTRAT | 516 | QQYYYWPIT | 579 |
| 9-C2_VL | RASQSVYNNLA | 439 | DASTRAT | 516 | QQYSDYPFT | 576 |
| 9-D8_VL | RASRSLSNNLA | 480 | DASTRAT | 516 | QQYNAWPYT | 580 |
| 9-E4_VL | RASQSVYNNLA | 439 | DASTRAT | 516 | QQYSDYPFT | 576 |
| 9-E5_VL | RASQSIANYLN | 481 | AASSLQS | 300 | QQYFSTETT | 581 |
| 9-G1_VL | RASQSIGNYLN | 482 | SASSLQS | 303 | QQTYTIPLT | 582 |
| 9-G3_VL | RASQSIATYLN | 474 | AASSLQS | 300 | QQSYTYPVT | 583 |
| 9-H9_VL | RASQSISSYLN | 445 | GASSLQS | 303 | QQSFTAPVT | 584 |
| 2-C8_VL | RASQTISRYLN | 450 | AASSLQS | 300 | QQSYTYPIT | 585 |
| 8-D9_VL | RASQTIGNYLN | 477 | VASSLQS | 313 | QQSYRYPYT | 573 |
| 10-A9_VL | RASQTISRYLN | 450 | SASSLQS | 303 | QQSYTYPIT | 585 |
| 10-B2_VL | RASQSISSYLN | 445 | AASSLQS | 300 | QQSYTIPPT | 565 |
| 10-B5_VL | RASQDISNYLN | 483 | AASSLQS | 300 | QQSYTYPLT | 531 |
| 10-C1_VL | RASQSISTYLN | 484 | AASSLQS | 300 | QQSYTFPLT | 529 |
| 10-C3_VL | RASQTIGIYLN | 483 | AASSLQS | 300 | QQSYTYPLT | 531 |
| 10-C9_VL | RASQTIGTYLN | 486 | AASSLQS | 300 | QQSYSYPLT | 523 |
| 10- | RASQSVSNNLA | 470 | GASTRAT | 501 | QQYNNWPYT | 586 |
| C11_VL | | | | | | |
| 10-D7_VL | RASQSISTYLN | 484 | AASSLQS | 300 | QQSYTVPLT | 548 |
| 10-E1_VL | RASQSISSYLN | 445 | AASSLQS | 300 | QQSYTIPPT | 565 |
| 10-E6_VL | RASQDIYNYLN | 487 | GASSLQS | 505 | QQSDGFPPT | 587 |
| 10-E7_VL | RASQSISNYLN | 438 | AASSLQS | 300 | QQSYTVPWT | 588 |
| 10- | RASQSVSSNLA | 488 | GASTRAT | 501 | QQYNDYPLT | 589 |
| F11_VL | | | | | | |
| 10- | RASQSISSNLA | 489 | DASTRAT | 516 | QQYYNWPLT | 566 |
| F12_VL | | | | | | |
| 10-G5_VL | RASQSVYSNLA | 490 | GASTRAT | 501 | QQYSNWPLT | 590 |
| 11-B6_VL | RASQSVGGNLA | 491 | DASTRAT | 516 | QQYNDWPLT | 535 |
| 11-D8_VL | RASQRISSYLN | 492 | AASSLQS | 300 | QQSYSYPIT | 567 |
| 11- | RASQSVASPYLA | 493 | GASSRAT | 502 | QQYYESPIT | 591 |
| D10_VL | | | | | | |
| 11- | RASQSIYNYLN | 494 | AASSLQS | 500 | QQSDSPPWT | 592 |
| D11_VL | | | | | | |
| 11-G2_VL | RASQSVSSSYLA | 454 | AASSRAT | 313 | QHYGGSPGYT | 563 |
| 11-G5_VL | RARQSVPSNYLA | 493 | GASSRAT | 502 | QQYYSSPLT | 593 |
| 11-G9_VL | RASQTISSYLN | 496 | SASSLQS | 303 | QQSYTFPLT | 529 |
| 11-H8_VL | RASQSISNYLN | 438 | AASSLQS | 300 | QQSYTVPLT | 548 |
| 11- | RASQSVSSSYLA | 454 | GASSRAT | 502 | QQYSSSPTYT | 594 |
| H11_VL | | | | | | |

### Example 5. Preferential Binding to PLXDC1 by Antibodies in Presence of Binding Compound

This example shows that compounds of the disclosure bind to the extracellular domain of PLXDC. Further, such binding by the small molecule compounds serve as an efficient and specific way for evaluation and screening of antibodies that activate PLXDC proteins to kill tumor endothelial cells.

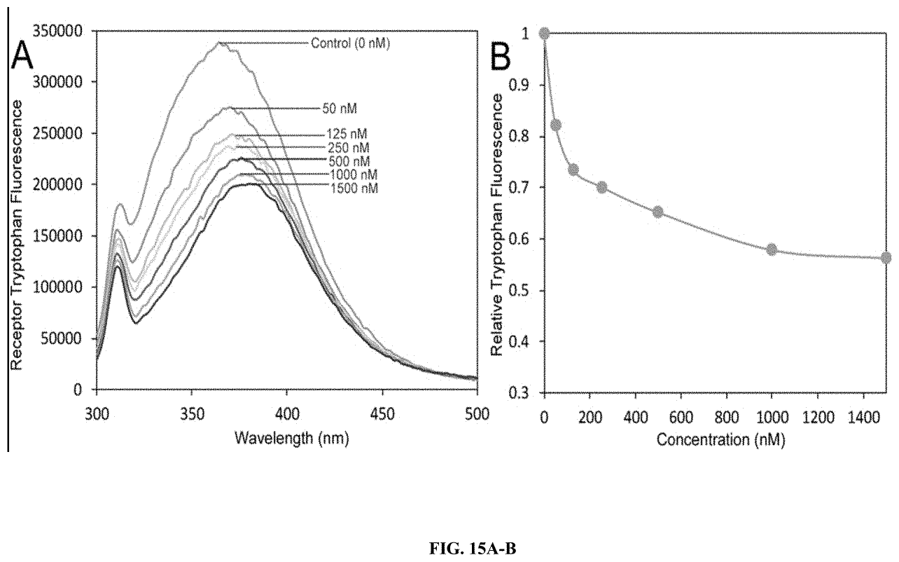

The high affinity interaction between compound 346 (Table 3) and the extracellular domain of PLXDC1 (PLXDC1-ECD). Compound 346 suppressed the endogenous tryptophan fluorescence of PLXDC1-ECD in a dose-dependent manner (FIG. 15A-B). FIG. 15A presents raw data of the tryptophan fluorescence of PLXDC1-ECD as measured in a fluorometer after adding different concentrations of the compound, and FIG. 15B shows the dose-dependent curve of the suppression of tryptophan fluorescence. Tryptophan fluorescence without the compound added is defined as 1. The estimated Kd value is 50 nM.

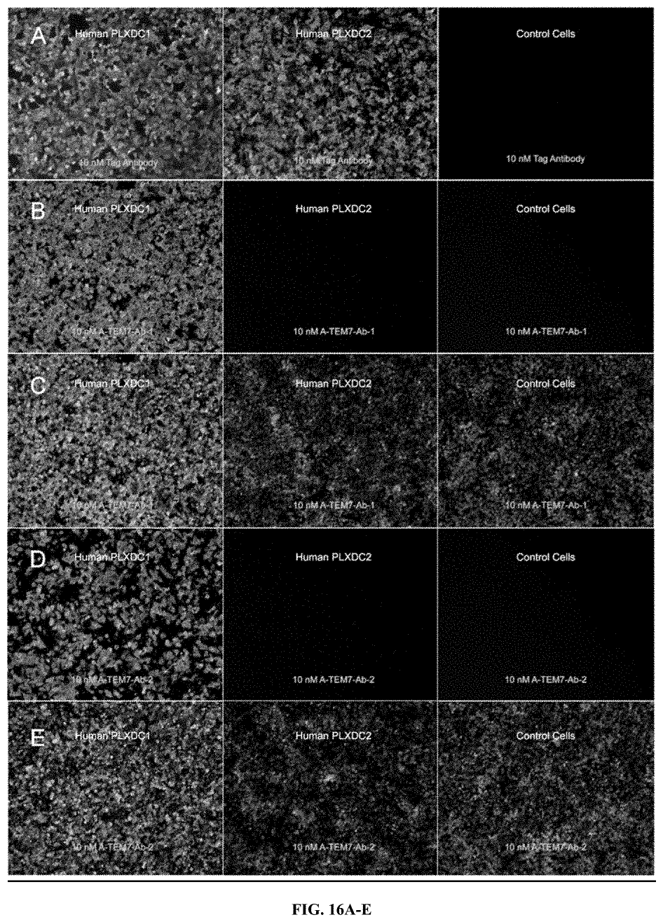

Binding of the antibodies (3-G7 and 8-C₉) to PLXDC expressed on live cells was also tested. Human PLXDC1 and PLXDC2 proteins were tagged with an epitope tag on the N-terminus. Binding of an antibody against the epitope tag demonstrates the expression of human PLXDC1 (FIG. 16A, left picture) and human PLXDC2 (FIG. 16A, middle picture) in their transfected cells, while it did not bind to untransfected cells (FIG. 16A, right picture).

Binding of antibody 3-G7 to cells transfected with human PLXDC1 is shown in FIG. 16A-B, left pictures. This antibody did not bind to human PLXDC2 (middle pictures) or untransfected control cells (right pictures). Likewise, FIG. 16D-E show the binding of antibody 8-C₉ to cells transfected with human PLXDC1 (left pictures) and this antibody did not bind to human PLXDC2 (middle pictures) or untransfected control cells (right pictures).

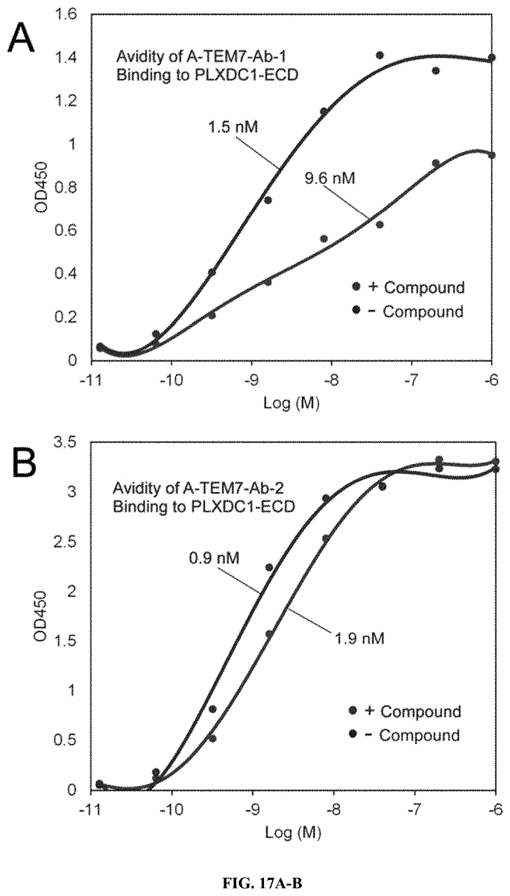

To verify that the activating antibodies preferably bind to the PLXDC1 protein in its activated state, bindings were measured by coating purified PLXDC1-ECD protein on the wells and adding serial dilution of antibodies. Compound 346 (Table 3) was added in the samples. As shown in FIG. 17A, antibody 3-G7 bound to PLXDC1-ECD with high avidity (9.6 nM) with the presence of compound 346. When compound 346 was added, the avidity was increased to 1.5 nM. Likewise, as shown in FIG. 17B, antibody 8-C₉ bound to PLXDC1-ECD with high avidity (1.9 nM) in the absence of compound 346, which was increased to 0.9 nM when the compound was added.

This example, therefore, validates the antibody-screening approach that employs compound-activated PLXDC proteins.

### Example 6. Killing of Tumor Endothelial Cells by PLXDC-Activating Compounds and Antibodies

This example investigates the mechanism by which the compounds and antibodies kill tumor endothelial cells, and demonstrates their killing activities.

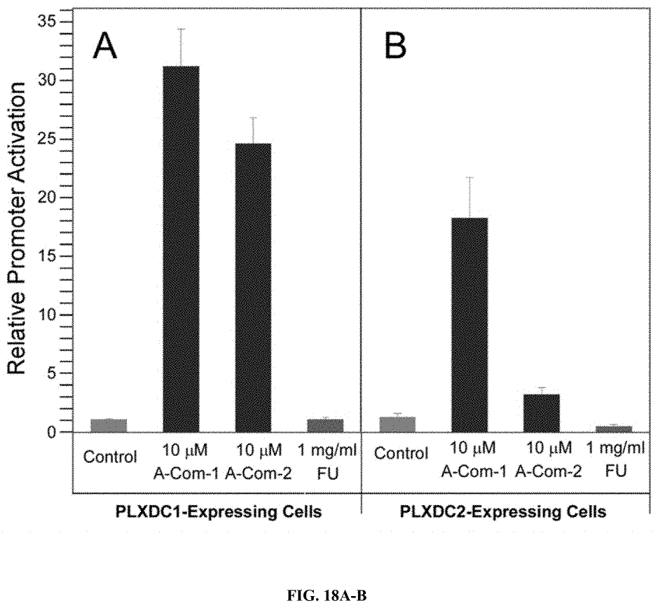

Through RNAseq analysis of PLXDC1-expressing endothelial cells killing by PLXDC1-activating compounds, this example a transcriptional factor called Gfi1b specifically induced during PLXDC1-mediated cell killing By linking its promotor to a luciferase reporter gene, this example developed a PLXDC1 receptor activation assay. PLXDC1-activating compounds 346 and 342 (Table 3, labeled as A-Compound-1 and A-Compound-2, respectively) highly activated the promotor activity in PLXDC1-expressing cells, but not in cells without PLXDC1 (FIG. 18A). Likewise, these compounds activated the promotor activity in PLXDC2-expressing cells, but not in cells without PLXDC2 (FIG. 18B).

FIG. 18 therefore shows that the compounds activated both PLXDC1 and PLXDC2 and that they preferentially activate PLXDC1 over PLXDC2. One of the compounds, A-Com-2, strongly differentiates between the two receptors. As a control, Fluorouracil, a chemotherapy agent that kills dividing cells by apoptosis does not activate this promoter. This data demonstrates that the cell death mediated by PLXDC1 activation is different from chemotherapy agent-triggered apoptosis.

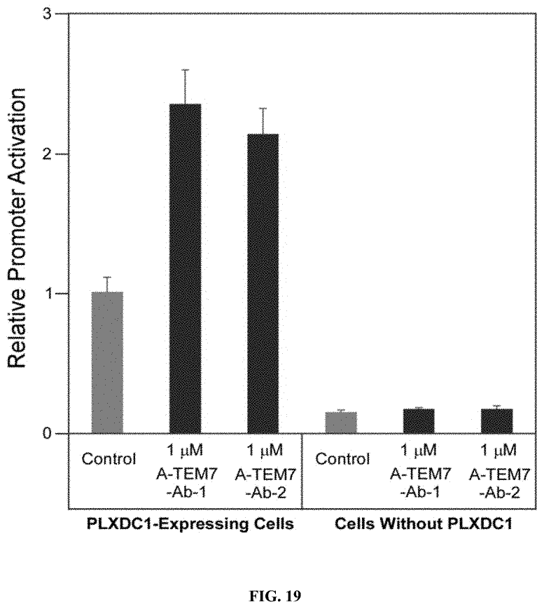

Activation of PLXDC1 by antibodies was demonstrated in FIG. 19. As shown, PLXDC1-activating antibodies 3-G7 and 8-C₉ (labeled as A-TEM7-Ab-1 and A-TEM7-Ab-2, respectively) activated the promotor activity in PLXDC1-expressing cells, but not in cells without PLXDC1.

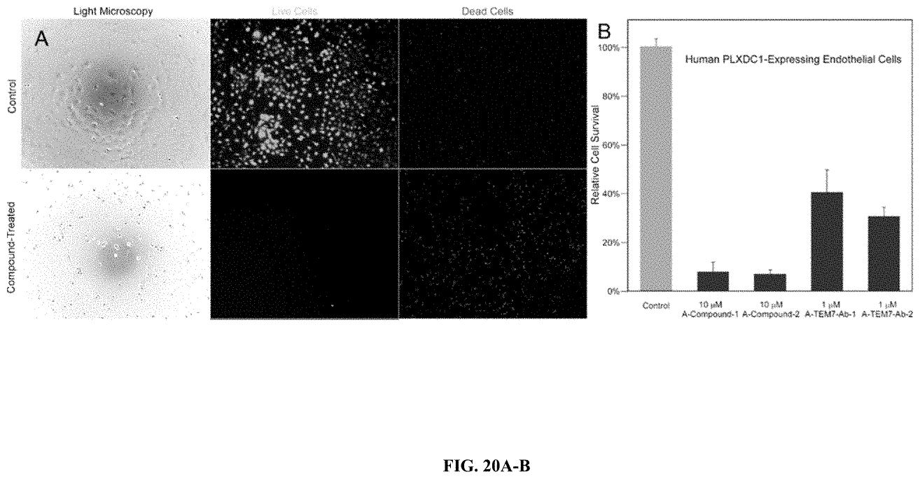

The killing of human PLXDC1-expressing endothelial cells by the compounds is visualized in FIG. 20. The top three pictures represent control cells and the lower three pictures represent compound-treated cells, showing light microscopy picture (FIG. 20A, left), live cell (middle) and dead cell staining (right). Live cells were stained using Fluorescein diacetate (green signal) and dead cells were stained using propidium iodide (red signal). Quantitation of the killing of human PLXDC1-expressing endothelial cells by the compounds and antibodies (antibodies 3-G7 and 8-C₉, labeled as A-TEM7-Ab-1 and A-TEM7-Ab-2, respectively) are shown in FIG. 20B. Incubation time of the compounds and antibodies was 24 hours.

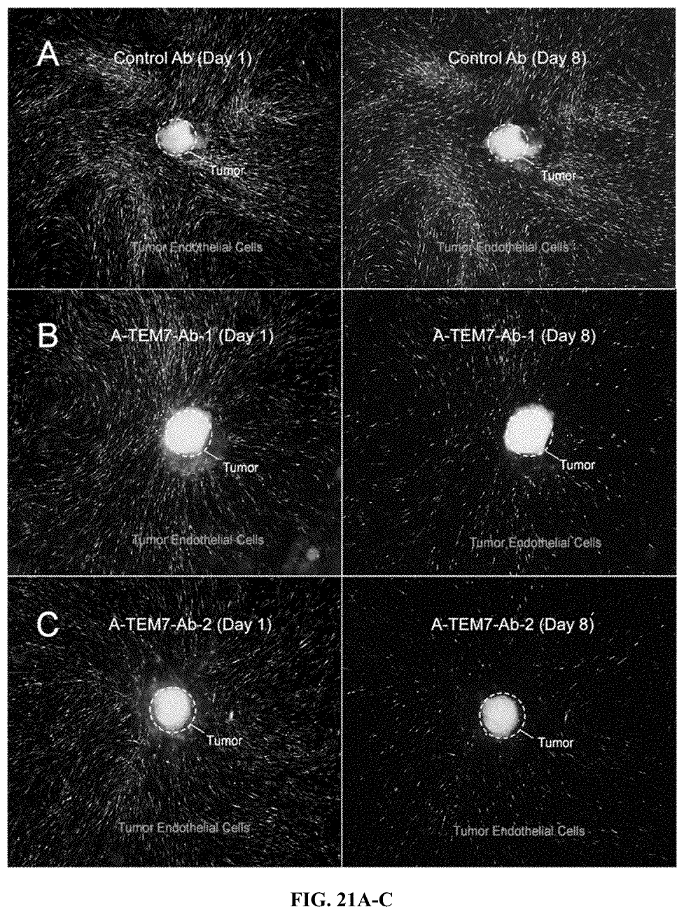

The killing of human tumor endothelial cells was also tested ex vivo. With the ex vivo model of tumor angiogenesis demonstrated in Example 1, antibodies were added after tumor endothelial cells grew out of the tumor (FIG. 21, marked in the middle). The pictures represent one day after antibody addition and the right pictures represent 8 days after antibody addition. Incubation of human lung tumor endothelial cells with control IgG (500 nM) did not lead to the death of tumor endothelial cells expressing PLXDC1 (FIG. 21, green signal). Incubation of human lung tumor endothelial cells with PLXDC1-activating IgG 3-G7/A-TEM7-Ab-1 (500 nM) led to the death of tumor endothelial cells expressing PLXDC1 (FIG. 21B, green signal), as evident by comparing the tumor endothelial cell signal between day 1 and day 8. Incubation of human lung tumor endothelial cells with PLXDC1-activativing IgG 8-C₉/A-TEM7-Ab-2 (500 nM) also led to the death of tumor endothelial cells expressing PLXDC1 (green signal), as evident by comparing the tumor endothelial cell signal between day 1 and day 8.

### Example 7. Specific Killing of Pathogenic Blood Vessels in Ischemia-Induced Retinopathy/Tumor

This example examines the expression of the PLXDC proteins on pathogenic blood vessels and normal healthy blood vessels, in different diseases, and confirms that the compounds and antibodies of the instant disclosure specifically kill the pathogenic blood vessels.

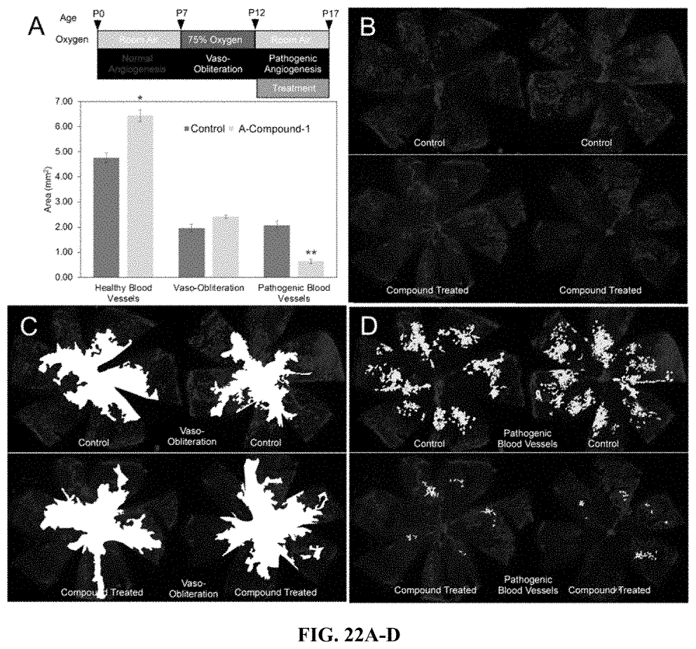

The expression of PLXDC1 in pathogenic blood vessels of ischemia-induced retinopathy was examined and shown in FIG. 1, which shows that PLXDC1 was not expressed in healthy blood vessels. It was then demonstrated that the compounds (e.g., compounds 346) specifically suppressed pathogenic blood vessels in vivo without affecting healthy blood vessels in ischemia-induced retinopathy (FIG. 22). FIG. 22A includes a schematic diagram of the experimental design for ischemia-induced retinopathy. The high oxygen environment caused blood vessel loss (vaso-obliteration). In room air, loss of vessels triggered abnormal angiogenesis that generated pathogenic blood vessels on the top of the retina (marked in yellow in FIG. 22D). Treatment was applied during the return to room air by subcutaneous injection. The lower graph in FIG. 22A shows quantitation of healthy blood vessels, vaso-obliteration and pathogenic blood vessels between the control (n=10) and treated retinas (n=10).

Treatment by PLXDC1-activating compound (compound 346/A-Compound-1) highly suppressed pathogenic blood vessels (two asterisks) while improving the amount of healthy blood vessels (one asterisk). FIG. 22B includes representative images of flat-mounted control retinas (upper two images) and retinas from compound treated mice (lower two images). The same retinas in B with vaso-obliteration areas marked in white color (FIG. 22C). These images illustrate that compound-treated retinas went through vaso-obliteration like the control retinas. The same retinas in B with pathogenic blood vessels marked in yellow color (FIG. 22D). These images illustrate that compound-treated retinas have highly decreased pathogenic blood vessels as compared to the control retinas.

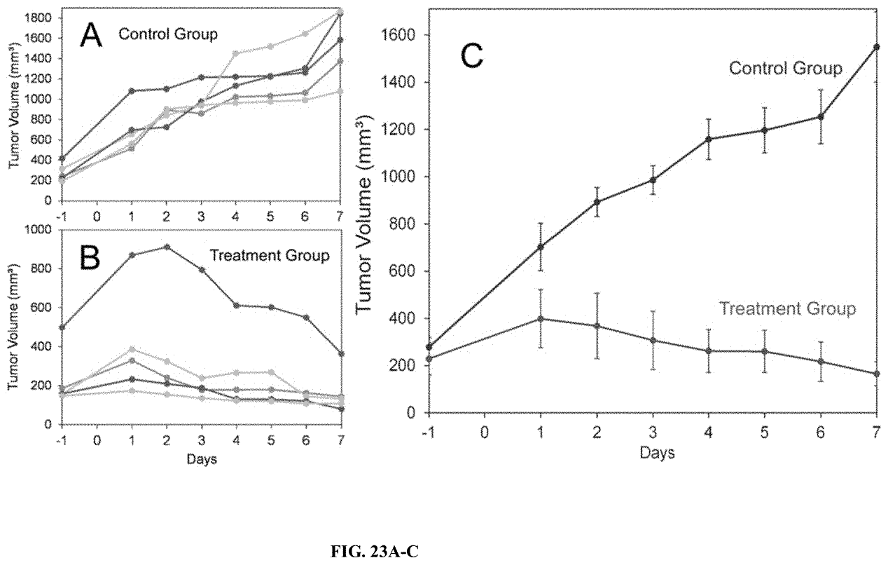

The killing activity was further demonstrated with tumor samples in vivo. Treatment was done at day 0 by bolus IV injection of compound 346 in a tumor animal model. FIG. 23A charts raw data of tumor growth curves of the mice in the control group. FIG. 23B presents raw data of tumor growth curves of the mice in the treatment group. FIG. 23C compares the combined growth data of the control group and the treatment group. Unlike in the control group, compound 346 shrank the tumors significantly.

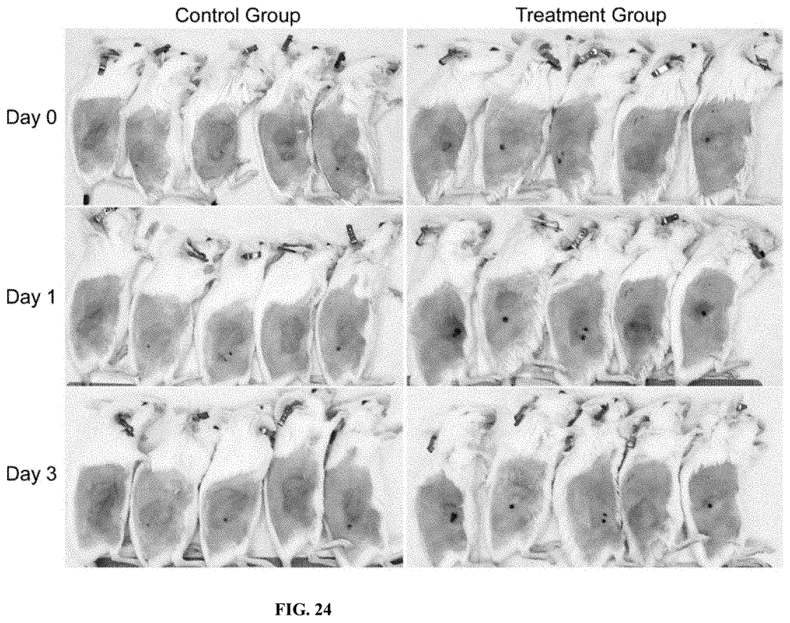

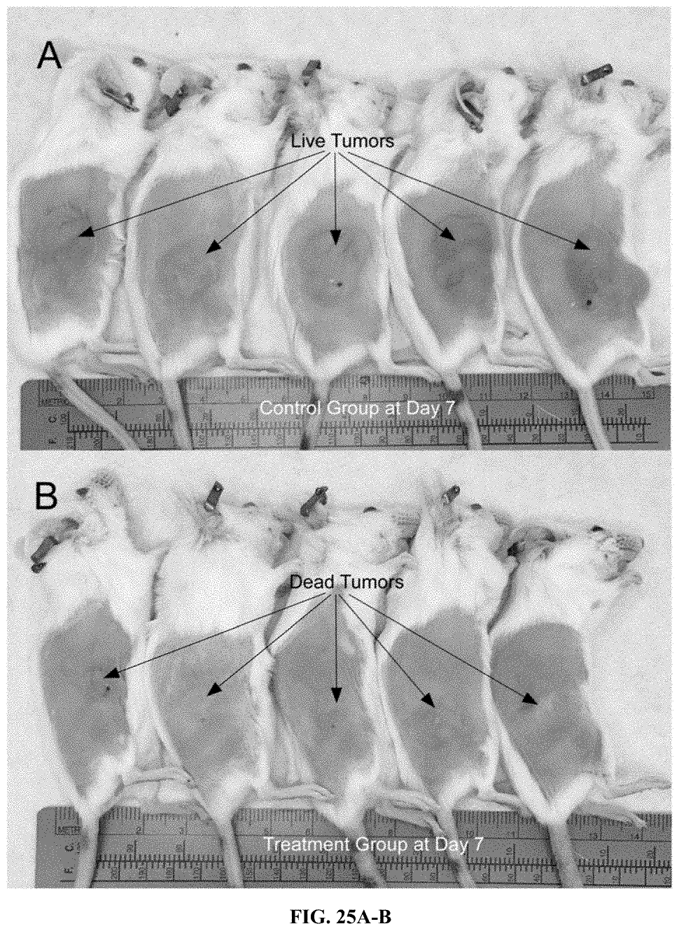

Tumor morphological changes on live animals due to the treatment by PLXDC1-activating compound were examined. Pictures of the whole animals in the experiment described in FIG. 25 show tumor morphological and color changes on day 1 and day 3 (FIG. 24). Tumors in the treatment groups becomes darker in color on day 1 due to the destruction of tumor blood vessels and accumulation of blood in the tumors. Tumors in the treatment groups started to become yellower in color on day 3, consistent with the onset of tumor necrosis due to the lack of tumor blood vessels.

FIG. 25 presents pictures of the whole animals showing tumor morphological and color changes on day 7. tumors in the control group have grown to large sizes, tumors in the treatment groups have highly shrunk in size and become yellow in color.

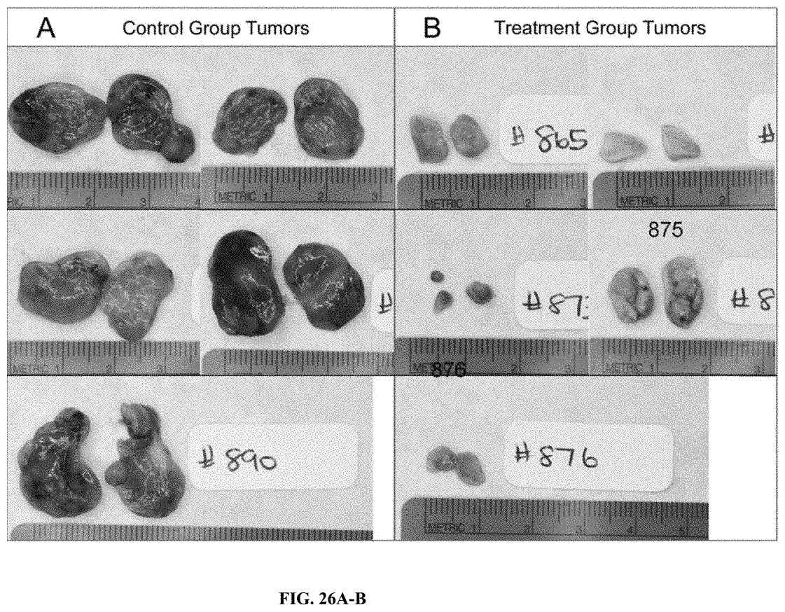

FIG. 26 shows morphological changes of dissected tumors due to the treatment by the compound. Pictures of the dissected tumors showed tumor morphological and color changes on day 7. While the tumors in the control group are reddish in color, tumors in the treatment groups had highly shrunk in size and become yellow in color, consistent with the lack of tumor blood vessels and tumor necrosis. These data, therefore, demonstrate that the PLXDC1-activating compounds can kill tumor blood vessels in vivo to cause strong tumor necrosis and shrinkage.

### Example 8. Activating Antibodies Interact with PLXDC1 Differently from PEDF

This example shows that the PLXDC1-activating antibodies 3-G7 and 8-C₉ (labeled as A-TEM7-Ab-1 and A-TEM7-Ab-2, respectively) do not require binding to domain B of PLXDC1, where PEDF binds.

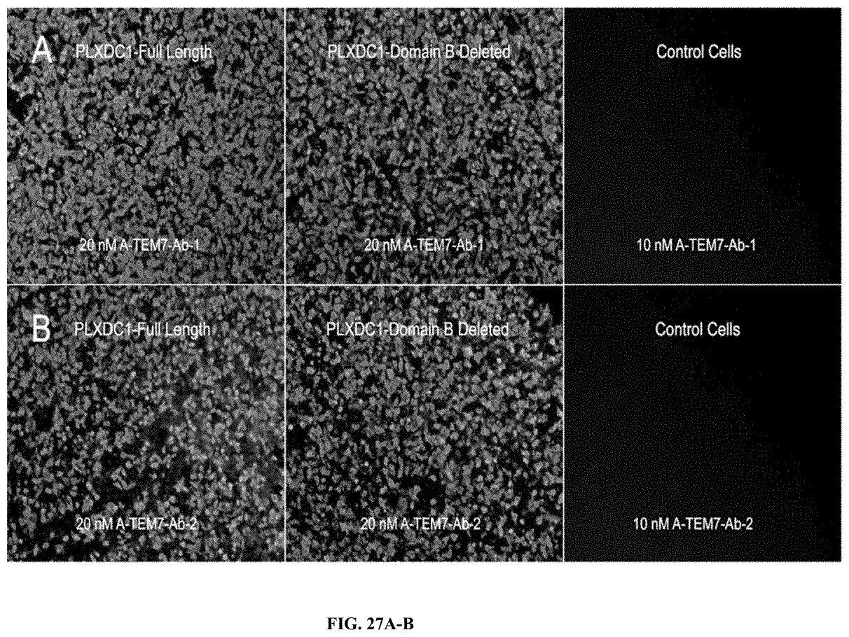

Example 1 already has demonstrated that antibodies AA02, AA03, and AA94 interacted with PLXDC1 even when domain B was deleted (FIG. 11). It is further demonstrated here that antibodies A-TEM7-Ab-1 and A-TEM7-Ab-2 also do not bind to PLXDC1 through domain B (FIG. 27A-B). This is in sharp contrast to PEDF, which depends on domain B to bind to PLXDC1. The fact that both small molecules and antibodies of the present disclosure do not depend on domain B to bind to PLXDC1 differentiate these novel ligands from PEDF and is consistent with the distinct activity of these new ligands (killing of tumor endothelial cells) that PEDF is not capable of doing.

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.
Beaty, R. M., Edwards, J. B., Boon, K., Siu, I. M., Conway, J. E., and Riggins, G. J. (2007). PLXDC1 (TEM7) is identified in a genome-wide expression screen of glioblastoma endothelium. J Neurooncol 81, 241-248.
Carmeliet, P. (2005). Angiogenesis in life, disease and medicine. Nature 438, 932-936.
Lee, H. K., Seo, I. A., Park, H. K., and Park, H. T. (2006). Identification of the basement membrane protein nidogen as a candidate ligand for tumor endothelial marker 7 in vitro and in vivo. FEBS Lett 580, 2253-2257.
Lu, C., Bonome, T., Li, Y., Kamat, A. A., Han, L. Y., Schmandt, R., Coleman, R. L., Gershenson,
D. M., Jaffe, R. B., Birrer, M. J., et al. (2007). Gene alterations identified by expression profiling in tumor-associated endothelial cells from invasive ovarian carcinoma. Cancer Res 67, 1757-1768.
Schwarze, S. R., Fu, V. X., Desotelle, J. A., Kenowski, M. L., and Jarrard, D. F. (2005). The identification of senescence-specific genes during the induction of senescence in prostate cancer cells. Neoplasia 7, 816-823.
St Croix, B., Rago, C., Velculescu, V., Traverso, G., Romans, K. E., Montgomery, E., Lal, A.,
Riggins, G. J., Lengauer, C., Vogelstein, B., et al. (2000). Genes expressed in human tumor endothelium. Science 289, 1197-1202.
van Beijnum, J. R., Petersen, K., and Griffioen, A. W. (2009). Tumor endothelium is characterized by a matrix remodeling signature. Front Biosci (Schol Ed) 1, 216-225.
Yamaji, Y., Yoshida, S., Ishikawa, K., Sengoku, A., Sato, K., Yoshida, A., Kuwahara, R., Ohuchida, K., Old, E., Enaida, H., et al. (2008). TEM7 (PLXDC1) in neovascular endothelial cells of fibrovascular membranes from patients with proliferative diabetic retinopathy. Invest Ophthalmol Vis Sci 49, 3151-3157.

1. A method for treating a disorder in a patient, wherein the disorder is characterized with pathogenic blood vessels and the method comprises activating a plexin domain-containing (PLXDC) protein expressed in the pathogenic blood vessels in the patient.

2. The method of claim 1, wherein the PLXDC protein comprises PLXDC1 or PLXDC2.

3. The method of claim 1, wherein activating the PLXDC protein comprises administration of an agent that binds to the PLXDC protein.

4. The method of claim 3, wherein the agent is a small molecule or a polypeptide.

5. The method of claim 4, wherein the agent is an anti-PLXDC antibody, or an antigen binding fragment thereof.

6. The method of claim 5, wherein the antibody or antigen binding fragment thereof is not capable of mediating antibody-dependent cell-mediated cytotoxicity (ADCC).

7. The method of claim 5, wherein the antibody or antigen binding fragment thereof binds to the PLXDC protein with a higher affinity in the presence of a small molecule compound that binds and activates the PLXDC protein, as compared to when the small molecule compound is not present.

8. The method of claim 5, wherein the antibody or antigen binding fragment thereof binds to Domain A, C, D, or E of PLXDC1.

9-12. (canceled)

13. The method of claim 5, wherein the antibody or antigen binding fragment thereof does not bind to Domain B of PLXDC1.

14. The method of claim 5, wherein the antibody or antigen binding fragment thereof is an antibody selected from Table 6 or an antigen binding fragment thereof, is an antibody or antigen binding fragment thereof that includes the complementarity-determining regions (CDR) VH CDR1, VH CDR2, VH CDR3, VL CDR1, VL CDR2 and VL CDR3 of the antibodies selected from Table 6, or is an antibody or antigen binding fragment thereof that competes with an antibody selected from Table 6 in binding to PLXDC1.

15. The method of claim 3, wherein the agent binds to an amino acid residue of PLXDC that is not exposed in the basal state.

16. The method of claim 3, wherein the agent is a small molecule compound.

17. The method of claim 16, wherein the compound is a compound of Formula I.

18. The method of claim 3, wherein the agent is not pigment epithelium-derived factor (PEDF) or a mimetic thereof.

19. The method of claim 1, wherein the disorder is selected from the group consisting of diabetic retinopathy, age-related macular degeneration (AMD), retinopathy of prematurity, cancer and combinations thereof.

20. The method of claim 19, wherein the disorder is a tumor.

21. The method of claim 20, wherein the tumor comprises a solid tumor.

22. The method of claim 20, wherein the tumor has a diameter of greater than 2 CM.

23. An antibody or antigen binding fragment thereof having specificity to the human plexin domain-containing 1 (PLXDC1) protein, wherein the antibody or antigen binding fragment thereof comprises a heavy chain variable region (VH) and a light chain variable region (VL), the VH comprises a VH complementarity-determining region (CDR) CDR1, a VH CDR2, a VH CDR3, the VL comprises a VL CDR1, a VL CDR2, and a VL CDR3, wherein the VH CDR1, VH CDR2, VH CDR3, VL CDR1, VL CDR2, and VL CDR3 comprises the VH CDR1, VH CDR2, VH CDR3, VL CDR1, VL CDR2, and VL CDR3, respectively, of an antibody selected from Table 6.

24-29. (canceled)

30. A method for identifying an activator of a PLXDC protein, comprising contacting a candidate molecule with the PLXDC protein in the presence of a reference PLXDC activator, and detecting the binding affinity between the candidate molecule and the PLXDC protein, thereby identifying the candidate molecule as a PLXDC activator when the detected binding affinity is greater than a reference binding affinity between the candidate molecule and the PLXDC protein in the absence of the reference PLXDC activator.

31-32. (canceled)


SEQUENCE LISTING (594 sequences)

SEQ ID NO: 1 - protein, 500 aa, Artificial Sequence
  Synthetic
MRGELWLLVLVLREAARALSPQPGAGHDEGPGSGWAAKGTVRGWNRRARESPGHVSEPDRTQLSQDLGGGTLAMDTLPDNRTRVVEDNHSYYVSRLYGPSEPHSRELWVDVAEANRSQVKIHTILSNTHRQASRVVLSFDFPFYGHPLRQITIATGGFIFMGDVIHRMLTATQYVAPLMANFNPGYSDNSTVVYFDNGTVFVVQWDHVYLQGWEDKGSFTFQAALHHDGRIVFAYKEIPMSVPEISSSQHPVKTGLSDAFMILNPSPDVPESRRRSIFEYHRIELDPSKVTSMSAVEFTPLPTCLQHRSCDACMSSDLTFNCSWCHVLQRCSSGFDRYRQEWMDYGCAQEAEGRMCEDFQDEDHDSASPDTSFSPYDGDLTTTSSSLFIDSLTTEDDTKLNPYAGGDGLQNNLSPKTKGTPVHLGTIVGIVLAVLLVAAIILAGIYINGHPTSNAALFFIERRPHHWPAMKFRSHPDHSTYAEVEPSGHEKEGFMEAEQC

SEQ ID NO: 2 - protein, 529 aa, Artificial Sequence
  Synthetic
MARFPKADLAAAGVMLLCHFFTDQFQFADGKPGDQILDWQYGVTQAFPHTEEEVEVDSHAYSHRWKRNLDFLKAVDTNRASVGQDSPEPRSFTDLLLDDGQDNNTQIEEDTDHNYYISRIYGPSDSASRDLWVNIDQMEKDKVKIHGILSNTHRQAARVNLSFDFPFYGHFLREITVATGGFIYTGEVVHRMLTATQYIAPLMANFDPSVSRNSTVRYFDNGTALVVQWDHVHLQDNYNLGSFTFQATLLMDGRIIFGYKEIPVLVTQISSTNHPVKVGLSDAFVVVHRIQQIPNVRRRTIYEYHRVELQMSKITNISAVEMTPLPTCLQFNRCGPCVSSQIGFNCSWCSKLQRCSSGFDRHRQDWVDSGCPEESKEKMCENTEPVETSSRTTTTVGATTTQFRVLTTTRRAVTSQFPTSLPTEDDTKIALHLKDNGASTDDSAAEKKGGTLHAGLIIGILILVLIVATAILVTVYMYHHPTSAASIFFIERRPSRWPAMKFRRGSGHPAYAEVEPVGEKEGFIVSEQC

SEQ ID NO: 3 - protein, 480 aa, Artificial Sequence
  Synthetic
MARFPKADLAAAGVMLLCHFFTDQFQFADGKPGDQILDWQYGVTQAFPHTEEEVEVDSHAYSHRWKRNLDFLKAVDTNRASVGQDSPEPRSFTDLLLDDGQDNNTQIERVNLSFDFPFYGHFLREITVATGGFIYTGEVVHRMLTATQYIAPLMANFDPSVSRNSTVRYFDNGTALVVQWDHVHLQDNYNLGSFTFQATLLMDGRIIFGYKEIPVLVTQISSTNHPVKVGLSDAFVVVHRIQQIPNVRRRTIYEYHRVELQMSKITNISAVEMTPLPTCLQFNRCGPCVSSQIGFNCSWCSKLQRCSSGFDRHRQDWVDSGCPEESKEKMCENTEPVETSSRTTTTVGATTTQFRVLTTTRRAVTSQFPTSLPTEDDTKIALHLKDNGASTDDSAAEKKGGTLHAGLIIGILILVLIVATAILVTVYMYHHPTSAASIFFIERRPSRWPAMKFRRGSGHPAYAEVEPVGEKEGFIVSEQC

SEQ ID NO: 4 - protein, 118 aa, Artificial Sequence
  Synthetic
EVQLLESGGGLVQPGGSLRLSCAASGFTFTSYAMSWVRQAPGKGLEWVSAISAGGGYTYYADSVKGRFTISRDNSKNTLYLQMNSLRAEDTAVYYCARYDYNSYFDSWGQGTLVTVSS

SEQ ID NO: 5 - protein, 109 aa, Artificial Sequence
  Synthetic
QSTQPPSVSGAPGQRVTISCTGSSSNIGAGYDVHWYQQLPGTAPKLLIYGNNNRPSGVPDRFSGSKSGTSASLAITGLQAEDEADYYCQSYDRSLSGWVFGGGTKLTVL

SEQ ID NO: 6 - protein, 121 aa, Artificial Sequence
  Synthetic
QVQLVESGGGVVQPGRSLRLSCAASGYAFSSYGMHWVRQAPGKGLEWVAVISHSGSNKYYANSVKGRFTISRDNSKNTLYLQMNSLRAEDTAVYYCARGLAGWEVYPFDVWGQGTLVTVSS

SEQ ID NO: 7 - protein, 109 aa, Artificial Sequence
  Synthetic
QSTQPPSVSGAPGQRVTISCTGSSSNIGAGFDVHWYQQLPGTAPKLLIYDNTNRPSGVPDRFSGSKSGTSASLAITGLQAEDEADYYCQSYDYSLRVWVFGGGTKLTVL

SEQ ID NO: 8 - protein, 124 aa, Artificial Sequence
  Synthetic
EVQLLESGGGLVQPGGSLRLSCAASGFTFSNYAMSWVRQAPGKGLEWVSTISGGGTYYADSVKGRFTISRDNSKNTLYLQMNSLRAEDTAVYYCARSRYWYTKLISYTYGMDVWGQGTLVTVSS

SEQ ID NO: 9 - protein, 111 aa, Artificial Sequence
  Synthetic
QSVLTQPPSVSGAPGQRVTISCTGSSSNIGATYDVHWYQQLPGTAPKLLIYVNNNRPSGVPDRFSGSKSGTSASLAITGLQAEDEADYYCQSYDSSLRAWVFGGGTKLTVL

SEQ ID NO: 10 - protein, 118 aa, Artificial Sequence
  Synthetic
QVQLVESGGGVVQPGRSLRLSCAASGFTFSNYGMHWVRQAPGKGLEWVAVISYHGRNEYYADSVKGRFTISRDNSKNTLYLQMNSLRAEDTAVYYCARGDDQDAFDPWGQGTLVTVSS

SEQ ID NO: 11 - protein, 107 aa, Artificial Sequence
  Synthetic
DIQMTQSPSSLSASVGDRVTITCRASQSISNYLNWYQQKPGKAPKLLIYAASSLQSGVPSRFSGSGSGTDFTLTISSLQPEDFATYYCQQSFSAPYTFGQGTKVEIK

SEQ ID NO: 12 - protein, 127 aa, Artificial Sequence
  Synthetic
QVQLVESGGGVVQPGRSLRLSCAASGFIFSDYDMHWVRQAPGKGLEWVAVISHSGSNKYYADSVKGRFTISRDNSKNTLYLQMNSLRAEDTAVYYCARDRSVYLYYGYHYYEGFDVWGQGTLVTVSS

SEQ ID NO: 13 - protein, 107 aa, Artificial Sequence
  Synthetic
EIVMTQSPATLSVSPGERATLSCRASQSVYNNLAWYQQKPGQAPRLLIYGASTRATGIPARFSGSGSGTEFTLTISSLQSEDFAVYYCQQYNDLPLTFGQGTKVEIK

SEQ ID NO: 14 - protein, 116 aa, Artificial Sequence
  Synthetic
EVQLLESGGGLVQPGGSLRLSCAASGFTFSDYAMSWVRQAPGKGLEWVSSIYTSGGRTYYADSVKGRFTISRDNSKNTLYLQMNSLRAEDTAVYYCARGYRYFDHWGQGTLVTVSS

SEQ ID NO: 15 - protein, 107 aa, Artificial Sequence
  Synthetic
EIVMTQSPATLSVSPGERATLSCRASQSVRNNLAWYQQKPGQAPRLLIYGASTRATGIPARFSGSGSGTEFTLTISSLQSEDFAVYYCQQYYTWPRTFGQGTKVEIK

SEQ ID NO: 16 - protein, 120 aa, Artificial Sequence
  Synthetic
EVQLLESGGGLVQPGGSLRLSCAASGFTFPNYAMSWVRQAPGKGLEWVSTIYGRGERTYYADSVKGRFTISRDNSKNTLYLQMNSLRAEDTAVYYCARVSVVWSYAFDYWGQGTLVTVSS

SEQ ID NO: 17 - protein, 107 aa, Artificial Sequence
  Synthetic
DIQMTQSPSSLSASVGDRVTITCRASQDISTYLNWYQQKPGKAPKLLIYAASSLQSGVPSRFSGSGSGTDFTLTISSLQPEDFATYYCQQSYSYPLTFGQGTKVEIK

SEQ ID NO: 18 - protein, 119 aa, Artificial Sequence
  Synthetic
QLQLQESGPGLVKPSETLSLTCTVSGGSISSSNYYWGWIRQPPGKGLEWIGSVYYTGRTYYNPSLKSRVTISVDTSKNQFSLRLSSVTAADTAVYYCARVFPYGAVDVWGQGTLVTVSS

SEQ ID NO: 19 - protein, 109 aa, Artificial Sequence
  Synthetic
EIVLTQSPGTLSLSPGERATLSCRASQSVSGSYLAWYQQKPGQAPRLLIYGASSRATGIPDRFSGSGSGTDFTLTISRLEPEDFAVYYCQQYGGSLPYTFGQGTKVEIK

SEQ ID NO: 20 - protein, 118 aa, Artificial Sequence
  Synthetic
QVQLVESGGGVVQPGRSLRLSCAASGFTFSDYGMHWVRQAPGKGLEWVAVISYEGSNEYYADSVKGRFTISRDNSKNTLYLQMNSLRAEDTAVYYCARGSEGSGFDVWGQGTLVTVSS

SEQ ID NO: 21 - protein, 107 aa, Artificial Sequence
  Synthetic
DIQMTQSPSSLSASVGDRVTITCRASQRISNYLNWYQQKPGKAPKLLIYAASSLQSGVPSRFSGSGSGTDFTLTISSLQPEDFATYYCQQSYSPPYTFGQGTKVEIK

SEQ ID NO: 22 - protein, 120 aa, Artificial Sequence
  Synthetic
EVQLLESGGGLVQPGGSLRLSCAASGFTFSTYAMSWVRQAPGKGLEWVSSIYGSGGKTYYADSVKGRFTISRDNSKNTLYLQMNSLRAEDTAVYYCARTDYSLYYQFDYWGQGTLVTVSS

SEQ ID NO: 23 - protein, 107 aa, Artificial Sequence
  Synthetic
DIQMTQSPSSLSASVGDRVTITCRASQSISRYLNWYQQKPGKAPKLLIYAASSLQSGVPSRFSGSGSGTDFTLTISSLQPEDLATYYCQQSYSYPVTFGQGTKVEIK

SEQ ID NO: 24 - protein, 118 aa, Artificial Sequence
  Synthetic
QVQLVESGGGVVQPGRSLRLSCAASGFTFTNYGMHWVRQAPGKGLEWVAVISFDGSNKYYANSVKGRFTISRDNSKNTLYLQMNSLRAEDTAVYYCARGGDEDGFDVWGQGTLVTVSS

SEQ ID NO: 25 - protein, 107 aa, Artificial Sequence
  Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYRPPYTFGQGTKVEIK

SEQ ID NO: 26 - protein, 129 aa, Artificial Sequence
  Synthetic
EVQLLESGGGLVQPGGSLRLSCAASGFTFSNYAMSWVRQAPGKGLEWVSSITGSGEYTNYADSVKGRFTISRDNSKNTLYLQMNSLRAEDTAVYYCAREVADYWDGGVYYYYDGGFDVWGQGTLVTVSS

SEQ ID NO: 27 - protein, 109 aa, Artificial Sequence
  Synthetic
EIVLTQSPGTLSLSPGERATLSCRASQSVSSNYLAWYQQKPGQAPRLLIYGASSRATGIPDRFSGSGSGTDFTLTISRLEPEDFAVYYCQQYGRSAPITFGQGTKVEIK

SEQ ID NO: 28 - protein, 120 aa, Artificial Sequence
  Synthetic
EVQLLESGGGLVQPGGSLRLSCAASGFTFSSYAMSWVRQAPGKGLEWVSTISGTGGRTYYADSVKGRFTISRDNSKNTLYLQMNSLRAEDTAVYYCARVDHYSGYAFDLWGQGTLVTVSS

SEQ ID NO: 29 - protein, 107 aa, Artificial Sequence
  Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSYPLTFGQGTKVEIK

SEQ ID NO: 30 - protein, 122 aa, Artificial Sequence
  Synthetic
EVQLLESGGGLVQPGGSLRLSCAASGFTFSSYAMSWVRQAPGKGLEWVSTIYGGGSYTYYADSVKGRFTISRDNSKNTLYLQMNSLRAEDTAVYYCARAEVDDYWYLYMDVWGQGTLVTVSS

SEQ ID NO: 31 - protein, 107 aa, Artificial Sequence
  Synthetic
DIQMTQSPSSLSASVGDRVTITCRASQDIGNYLNWYQQKPGKAPKLLIYAASSLQSGVPSRFSGSGSGTDFTLTISSLQPEDFATYYCQQSYTFPLTFGQGTKVEIK

SEQ ID NO: 32 - protein, 120 aa, Artificial Sequence
  Synthetic
EVQLLESGGGLVQPGGSLRLSCAASGFTFSTYAMSWVRQAPGKGLEWVSTIYGSGGRTYYADSVKGRFTISRDNSKNTLYLQMNSLRAEDTAVYYCARTDHYSYYVMDYWGQGTLVTVSS

SEQ ID NO: 33 - protein, 107 aa, Artificial Sequence
  Synthetic
DIQMTQSPSSLSASVGDRVTITCRASQSISKYLNWYQQKPGKAPKLLIYSASSLQSGVPSRFSGSGSGTDFTLTISSLQPEDCATYYCQQSYSFPLTFGQGTKVEIK

SEQ ID NO: 34 - protein, 121 aa, Artificial Sequence
  Synthetic
EVQLLESGGGLVQPGGSLRLSCAASGFTFSDYAMSWVRQAPGKGLEWVSAIYGSGSYTYYADSVKGRFTISRDNSKNTLYLQMNSLRAEDTAVYYCARTAYSYPYVYFDFWGQGTLVTVSS

SEQ ID NO: 35 - protein, 107 aa, Artificial Sequence
  Synthetic
DIQMTQSPSSLSASVGDRVTITCRASQTITSYLNWYQQKPGKAPKLLIYAASSLQSGVPSRFSGSGSGTDFTLTISSLQPEDFATYYCQQSYTYPLTFGQGTKVEIK

SEQ ID NO: 36 - protein, 126 aa, Artificial Sequence
  Synthetic
EVQLLESGGGLVQPGGSLRLSCAASGFTFGNYAMSWVRQAPGKGLEWVSVISGGGTNTYYADSVKGRFTISRDNSKNTLYLQMNSLRAEDTAVYYCARSDSYYGVYDGYYYGMDVWGQGTLVTVSS

SEQ ID NO: 37 - protein, 107 aa, Artificial Sequence
  Synthetic
DIQMTQSPSSLSASVGDRVTITCRASQTISRYLNWHQQKPGKAPKLLIYAASSLQSGVPSRFSGSGSGTDFTLTISSLQPEDFATYYCQQTYSTPWTFGQGTKVEIK

SEQ ID NO: 38 - protein, 119 aa, Artificial Sequence
  Synthetic
EVQLLESGGGLVQPGGSLRLSCAASGFTFSSYAMSWVRQAPGKGLEWVSTIYASGATTYYADSVKGRFTISRDNSKNTLYLQMNSLRAEDTAVYYCARLKLYHAAFDIWGQGTLVTVSS

SEQ ID NO: 39 - protein, 107 aa, Artificial Sequence
  Synthetic
DIQMTQSPSSLSASVGDRVTITCRASQDISSYLNWYQQKPGKAPKLLIYTASSLQSGVPSRFSGSGSGTDFTLTISSLQPEDFATYYCQQSYSYPLTFGQGTKVEIK

SEQ ID NO: 40 - protein, 120 aa, Artificial Sequence
  Synthetic
EVQLLESGGGLVQPGGSLRLSCAASGFTFRDYAMSWVRQAPGKGLEWVSTIYGSGSRTYYADSVKGRFTISRDNSKNTLYLQMNSLRAEDTAVYYCARVDHVHGYAFDYWGQGTLVTVSS

SEQ ID NO: 41 - protein, 107 aa, Artificial Sequence
  Synthetic
DIQMTQSPSSLSASVGDRVTITCRASQSISKYLNWYQQKPGKAPKLLIYAASSLQSGVPSRFSGSGSGTDFTLTISSLQPEDFATYYCQQSYSFPPTFGQGTKVEIK

SEQ ID NO: 42 - protein, 128 aa, Artificial Sequence
  Synthetic
EVQLLESGGGLVQPGGSLRLSCAASGFTFSNYAMSWVRQAPGKGLEWVSTIYESGGYTYYADSVKGRFTISRDNSKNTLYLQMNSLRAEDTAVYYCARVESYGYGEYITYNYYGFDVWGQGTLVTVSS

SEQ ID NO: 43 - protein, 109 aa, Artificial Sequence
  Synthetic
EIVLTQSPGTLSLSPGERATLSCRASQSVATGYLAWYQQKPGQAPRLLIYGASSRATGIPDRFSGSGSGTDFTLTISRLEPEDFAVYYCQQYESSPPFTFGQGTKVEIK

SEQ ID NO: 44 - protein, 127 aa, Artificial Sequence
  Synthetic
QVQLVQSGAEVKKPGSSVKVSCKASGGTFTSYGIHWVRQAPGQGLEWMGRIVPILGTTNYAQKFQGRVTITADESTSTAYMELSSLRSEDTAVYYCARDPAVTLDTYVYADHGFDVWGQGTLVTVSS

SEQ ID NO: 45 - protein, 107 aa, Artificial Sequence
  Synthetic
EIVMTQSPATLSVSPGERATLSCRASQSVYNNLAWYQQKPGQAPRLLIYGASTRATGIPARFSGSGSGTEFTLTISSLQSEDFAVYYCQQYNDWPLTFGQGTKVEIK

SEQ ID NO: 46 - protein, 126 aa, Artificial Sequence
  Synthetic
EVQLLESGGGLVQPGGSLRLSCAASGFTFKNYAMSWVRQAPGKGLEWVSGISEGGANTYYADSVKGRFTISRDNSKNTLYLQMNSLRAEDTAVYYCAREPPKYGAYSYYGYGFDPWGQGTLVTVSS

SEQ ID NO: 47 - protein, 108 aa, Artificial Sequence
  Synthetic
EIVLTQSPGTLSLSPGERATLSCRASQSVGSNYLAWYQQKPGQAPRLLIYGASSRATGIPDRFSGSGSGTDFTLTISRLEPEDFAVYYCQQYGSSPYTFGQGTKVEIK

SEQ ID NO: 48 - protein, 118 aa, Artificial Sequence
  Synthetic
EVQLLESGGGLVQPGGSLRLSCAASGFTFKGYAMSWVRQAPGKGLEWVSSISVSGAGTYYADSVKGRFTISRDNSKNTLYLQMNSLRAEDTAVYYCARYVRGVSFDVWGQGTLVTVSS

SEQ ID NO: 49 - protein, 109 aa, Artificial Sequence
  Synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQYETSPPVTFGQGTKVEIK

SEQ ID NO: 50 - protein, 119 aa, Artificial Sequence
  Synthetic
EVQLLESGGGLVQPGGSLRLSCAASGFTFSNYAMSWVRQAPGKGLEWVSAIGGSGGGTYYADSVKGRFTISRDNSKNTLYLQMNSLRAEDTAVYYCARYGWEYQAFDYWGQGTLVTVSS

SEQ ID NO: 51 - protein, 107 aa, Artificial Sequence
  Synthetic
DIQMTQSPSSLSASVGDRVTITCRASQSISRYLNWYQQKPGKAPKLLIYAASSLQSGVPSRFSGSGSGTDFTLTISSLQPEDFATYYCQQSYSTPWTFGQGTKVEIK

SEQ ID NO: 52 - protein, 119 aa, Artificial Sequence
  Synthetic
EVQLLESGGGLVQPGGSLRLSCAASGFTFSNYAMSWVRQAPGKGLEWVSGITAGGGNTYYADSVKGRFTISRDNSKNTLYLQMNSLRAEDTAVYYCARIAGLFDGFDVWGQGTLVTVSS

SEQ ID NO: 53 - protein, 108 aa, Artificial Sequence
  Synthetic
EIVLTQSPGTLSLSPGERATLSCRASQSVRSSYLAWYQQKPGQAPRLLIYGASSRATGIPDRFSGSGSGTDFTLTISRLEPEDFAVYYCQQYASLPLTFGQGTKVEIK

SEQ ID NO: 54 - protein, 119 aa, Artificial Sequence
  Synthetic
EVQLLESGGGLVQPGGSLRLSCAASGFTFSNYAMSWVRQAPGKGLEWVSGISASGGGTYYADSVKGRFTISRDNSKNTLYLQMNSLRAEDTAVYYCARSSRYGDTFDVWGQGTLVTVSS

SEQ ID NO: 55 - protein, 107 aa, Artificial Sequence
  Synthetic
EIVMTQSPATLSVSPGERATLSCRASLSVGSNLAWYQQKPGQAPRLLIYGASTRATGIPARFSGSGSGTEFTLTISSLQSEDFAVYYCQQYNNLPLTFGQGTKVEIK

SEQ ID NO: 56 - protein, 124 aa, Artificial Sequence
  Synthetic
EVQLLESGGGLVQPGGSLRLSCAASGFTFTDYAMSWVRQAPGKGLEWVSGISVSGGYTYYADSVKGRFTISRDNSKNTLYLQMNSLRAEDTAVYYCARGLYAHHTYYYGGFDYWGQGTLVTVSS

SEQ ID NO: 57 - protein, 107 aa, Artificial Sequence
  Synthetic
EIVMTQSPATLSVSPGERATLSCRASQSVGSNLAWYQQKPGQAPRLLIYGASTRATGIPARFSGSGSGTEFTLTISSLQSEDFAVYYCQQYHNWPPTFGQGTKVEIK

SEQ ID NO: 58 - protein, 127 aa, Artificial Sequence
  Synthetic
EVQLLESGGGLVQPGGSLRLSCAASGFTFRSYAMSWVRQAPGKGLEWVSTIYGTGEDTYYADSVKGRFTISRDNSKNTLYLQMNSLRAEDTAVYYCARDYFWSPRGSIYDYDYFDVWGQGTLVTVSS

SEQ ID NO: 59 - protein, 107 aa, Artificial Sequence
  Synthetic
DIQMTQSPSSLSASVGDRVTITCRASQSISRYLNWYQQKPGKAPKLLIYSASSLQSGVPSRFSGSGSGTDFTLTISSLQPEDFATYYCQQYFSAPYTFGQGTKVEIK

SEQ ID NO: 60 - protein, 118 aa, Artificial Sequence; identical to SEQ ID NO: 48
  Synthetic

SEQ ID NO: 61 - protein, 109 aa, Artificial Sequence
  Synthetic
EIVLTQSPGTLSLSPGERATLSCRASQSVSSNYLAWYQQKPGQAPRLLIYGASSRATGIPDRFSGSGSGTDFTLTISRLEPEDFAVYYCQQYGSSPAVTFGQGTKVEIK

SEQ ID NO: 62 - protein, 127 aa, Artificial Sequence
  Synthetic
EVQLLESGGGLVQPGGSLRLSCAASGFTFSDYAMSWVRQAPGKGLEWVSAISSSGGTTYYADSVKGRFTISRDNSKNTLYLQMNSLRAEDTAVYYCARDAGWSYYGLWIYYYYMDVWGQGTLVTVSS

SEQ ID NO: 63 - protein, 108 aa, Artificial Sequence
  Synthetic
EIVLTQSPGTLSLSPGERATLSCRASQSVRSNYLAWYQQKPGQAPRLLIYGASSRATGIPDRFSGSGSGTDFTLTISRLEPEDFAVYYCQQYGASPQTFGQGTKVEIK

SEQ ID NO: 64 - protein, 124 aa, Artificial Sequence
  Synthetic
EVQLLESGGGLVQPGGSLRLSCAASGFTFKDYAMSWVRQAPGKGLEWVSGISGSGGYTYYADSVKGRFTISRDNSKNTLYLQMNSLRAEDTAVYYCARKLGGFDENYTYGMDVWGQGTLVTVSS

SEQ ID NO: 65 - protein, 109 aa, Artificial Sequence
  Synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPEYTFGQGTKVEIK

SEQ ID NO: 66 - protein, 121 aa, Artificial Sequence
  Synthetic
EVQLLESGGGLVQPGGSLRLSCAASGFTFSTYAMSWVRQAPGKGLEWVSGISAGGGARTYYADSVKGRFTISRDNSKNTLYLQMNSLRAEDTAVYYCARVHHFGYYSLDVWGQGTLVTVSS

SEQ ID NO: 67 - protein, 107 aa, Artificial Sequence
  Synthetic
DIQMTQSPSSLSASVGDRVTITCRASQTISNYLNWYQQKPGKAPKLLIYAASSLQSGVPSRFSGSGSGTDFTLTISSLQPEDFATYYCQQSYTYPLTFGQGTKVEIK

SEQ ID NO: 68 - protein, 118 aa, Artificial Sequence
  Synthetic
EVQLLESGGGLVQPGGSLRLSCAASGFTFSSYAMSWVRQAPGKGLEWVSVIYGSGGRTYYADSVKGRFTISRDNSKNTLYLQMNSLRAEDTAVYYCARHDPRYQLDVWGQGTLVTVSS

SEQ ID NO: 69 - protein, 107 aa, Artificial Sequence
  Synthetic
DIQMTQSPSSLSASVGDRVTITCRASQSIGSYLNWYQQKPGKAPKLLIYTASSLQSGVPSRFSGSGSGTDFTLTISSLQPEDFATYYCQQSYTYPLTFGQGTKVEIK

SEQ ID NO: 70 - protein, 118 aa, Artificial Sequence
  Synthetic
QVQLVESGGGVVQPGRSLRLSCAASGFTFSDYGMHWVRQAPGKGLEWVAVISYDGSRKYYANSVKGRFTISRDNSKNTLYLQMNSLRAEDTAVYYCARGSYSDGFDVWGQGTLVTVSS

SEQ ID NO: 71 - protein, 107 aa, Artificial Sequence
  Synthetic
DIQMTQSPSSLSASVGDRVTITCRASQSISRYLNWYQQKTGKAPKLLIYGASSLQSGVPSRFSGSGSGTDFTLTISSLQPEDFATYYCQQSYSTPYTFGQGTKVEIK

SEQ ID NO: 72 - protein, 125 aa, Artificial Sequence
  Synthetic
QVQLVQSGAEVKKPGSSVKVSCKASGGTFSTEYISWVRQAPGQGLEWMGRIIPVLGITNYAQKFQGRVTITADESTSTAYMELSSLRSEDTAVYYCARHLGPPYLVTYSHGFDVWGQGTLVTVSS

SEQ ID NO: 73 - protein, 107 aa, Artificial Sequence
  Synthetic
EIVMTQSPATLSVSPGERATLSCRASQSLGTNLAWYQQKPGQAPRLLIYGASTRATGIPARFSGSGSGTEFTLTISSLQSEDFAVYYCQQYYEAPPTFGQGTKVEIK

SEQ ID NO: 74 - protein, 119 aa, Artificial Sequence
  Synthetic
EVQLLESGGGLVQPGGSLRLSCAASGFTFSNYAMSWVRQAPGKGLEWVSAIYGSGGKTYYADSVKGRFTISRDNSKNTLYLQMNSLRAEDTAVYYCARLKYGFYQFDYWGQGTLVTVSS

SEQ ID NO: 75 - protein, 107 aa, Artificial Sequence
  Synthetic
DIQMTQSPSSLSASVGDRVTITCRASQSIRNYLNWYEQKPGKAPKLLIYAASSLQSGVPSRFSGSGSGTDFTLTISSLQPEDFATYYCQQSYSYPLTFGQGTKVEIK

SEQ ID NO: 76 - protein, 120 aa, Artificial Sequence
  Synthetic
EVQLLESGGGLVQPGGSLKLSCAASGFTFSNYAMSWVRQAPGKGLEWVSVIYAGGARTYYADSVKGRFTISRDNSKNTLYLQMNSLRAEDTAVYYCARWGGDGFYAMDYWGQGTLVTVSS

SEQ ID NO: 77 - protein, 107 aa, Artificial Sequence
  Synthetic
DIQMTQSPSSLSASVGDRVTITCRASQTISTYLNWYQQKPGKAPKLLIYAASILQSGVPSRFSGSGSGTDFTLTISSLQPEDFATYYCQQSYTVPLTFGQGTKVEIK

SEQ ID NO: 78 - protein, 124 aa, Artificial Sequence
  Synthetic
EVQLLESGGGLVQPGGSLRLSCAASGFTFSNYAMSWVRQAPGKGLEWVSAISGSGGTTYYADSVKGRFTISRDNSKNTLYLQMNSLRAEDTAVYYCARIKKYGSAGYTVGFDSWGQGTLVTVSS

SEQ ID NO: 79 - protein, 107 aa, Artificial Sequence
  Synthetic
EIVMTQSPATLSVSPGERATLSCRASQSVGSYLAWYQQKPGQAPRLLIYGASTRATGIPARFSGSGSGTEFTLTISSLQSEDFAVYYCQQYYNAPITFGQGTKVEIK

SEQ ID NO: 80 - protein, 124 aa, Artificial Sequence
  Synthetic
QLQLQESGPGLVKPSETLSLTCTVSGGSISSSSYYWGWIRQPPGKGLEWIGNIYYTGTTYYNPSLKSRVTISVDTSKNQFSLKLSSVTAADTAVYYCARIKVPWYYSSYYFDYWGQGTLVTVSS

SEQ ID NO: 81 - protein, 111 aa, Artificial Sequence
  Synthetic
QSVLTQPPSVSGAPGQRVTISCTGSSSNIGAGYDVHWYQQLPGTAPKLLIYGNKNRPSGVPDRFSGSKSGTSASLAITGLQAEDEADYYCQSYDTSLSAWVFGGGTKLTVL

SEQ ID NO: 82 - protein, 117 aa, Artificial Sequence
  Synthetic
EVQLLESGGGLVQPGGSLRLSCAASGFTFKNYAMSWVRQAPGKGLEWVSAISASGGYTYYADSVKGRFTISRDNSKNTLYLQMNSLRAEDTAVYYCARGRWGAFDYWGQGTLVTVSS

SEQ ID NO: 83 - protein, 111 aa, Artificial Sequence
  Synthetic
QSVLTQPPSVSGAPGQRVTISCTGSSSNIGSGYDVHWYQQLPGTAPKLLIYGNRNRPSGVPDRFSGSKSGTSASLAITGLQAEDEADYYCQSYDSSLKTWVFGGGTKLTVL

SEQ ID NO: 84 - protein, 117 aa, Artificial Sequence
  Synthetic
EVQLLESGGGLVQPGGSLKLSCAASGFTFSDYAMSWVRQAPGKGLEWVSGISRGGARTYYADSVKGRFTISRDNSKNTLYLQMNSLRAEDTAVYYCARYLFGHYMDWGQGTLVTVSS

SEQ ID NO: 85 - protein, 111 aa, Artificial Sequence
  Synthetic
QSVLTQPPSVSGAPGQRVTISCTGSSSNIGAGYDVHWYQQLPGTAPKLLIYGNTNRPSGVPDRFSGSKSGTSASLAITGLQAEDEADYYCQSYDGRLGVSVFGGGTKLTVL

SEQ ID NO: 86 - protein, 118 aa, Artificial Sequence
  Synthetic
QVQLVESGGGVVQPGRSLRLSCAASGFTFSRYAMHWVRQAPGKGLEWVAVISHSGSTKYYANSVKGRFTISRDNSKNTLYLQMNSLRAEDTAVYYCARSRGWRGFDSWGQGTLVTVSS

SEQ ID NO: 87 - protein, 110 aa, Artificial Sequence
  Synthetic
QSVLTQPPSVSGAPGQRVTISCTGSSSNIGAYVVHWYQQLPGTAPKLLIYGNTNRPSGVPDRFSGSKSGTSASLAITGLQAEDEADYYCQSYDRTLSSYVFGGGTKLTVL

SEQ ID NO: 88 - protein, 127 aa, Artificial Sequence
  Synthetic
QVQLVESGGGVVQPGRSLRLSCAASGFAFSGYGMHWVRQAPGKGLEWVAVISHHGSYKYYANSVKGRFTISRDNSKNTLYLQMNSLRAEDTAVYYCARGGADVGYFYSTFYYYMDVWGQGTLVTVSS

SEQ ID NO: 89 - protein, 111 aa, Artificial Sequence
  Synthetic
QSVLTQPPSVSGAPGQRVTISCTGSSSNIGAAFDVHWYQQLPGTAPKLLIYDNYNRPSGVPDRFSGSKSGTSASLAITGLQAEDEADYYCQSYDSSLSVWVFGGGTKLTVL

SEQ ID NO: 90 - protein, 120 aa, Artificial Sequence
  Synthetic
EVQLLESGGGLVQPGGSLRLSCAASGFTFSNYAMSWVRQAPGKGLEWVSAISRGGGYTYYADSVKGRFTISRDNSKNTLYLQMNSLRAEDTAVYYCARWGGDGFYAMDYWGQGTLVTVSS

SEQ ID NO: 91 - protein, 111 aa, Artificial Sequence
  Synthetic
QSVLTQPPSVSGAPGQRVTISCTGSSSNIGAGYDVHWYQQLPGTAPKLLIYANNNRPSGVPDRFSGSKSGTSASLAITGLQAEDEADYYCQSYDYSLSGWVFGGGTKLTVL

SEQ ID NO: 92 - protein, 122 aa, Artificial Sequence
  Synthetic
QLQLQESGPGLVKPSETLSLTCTVSGGSISSRGYYWAWIKQPPGKGLEWIGSIYYSGSTYYNPSLKSRVTISVDTSKNQFSLKLSSVTAADTAVYYCARLVSYGAYYVFDYWGQGTLVTVSS

SEQ ID NO: 93 - protein, 111 aa, Artificial Sequence
  Synthetic
QSVLTQPPSVSGAPGQRVTISCTGSSSNIGAGYDVHWYQQLPGTAPKLLIYGNRNRPSGVPDRFSGSKSGTSASLAITGLQAEDEADYYCQSYDTRLSAWVFGGGTKLTVL

SEQ ID NO: 94 - protein, 115 aa, Artificial Sequence
  Synthetic
EVQLLESGGGLVRPGGSLRLSCAASGFTFSSYAMSWVRQAPGKGLEWVSAISGSGGSTYYADSVKGRFTISRDNSKNTLYLQMNSLRAEDTAVYYCARDKRLTYWGQGTMVTVSS

SEQ ID NO: 95 - protein, 110 aa, Artificial Sequence
  Synthetic
QSVVTQPPSVSAAPGQKVTISCSGSSSNIGNNYVSWYQQLPGTAPKLLIYDNNKRPSGIPDRFSGSKSGTSATLGITGLQTGDEADYYCGTWDSSLSARVFGGGTKVTVL

SEQ ID NO: 96 - protein, 118 aa, Artificial Sequence
  Synthetic
QVQLVQSGAEVKKPGSSVKVSCKASGGTFSSYAINWVRQAPGQGLEWMGRIIPLLETADYAQKFQGRVTITADESTSTAYMELSSLRSEDTAVYYCAREWPGEYFDVWGQGTLVTVSS

SEQ ID NO: 97 - protein, 108 aa, Artificial Sequence
  Synthetic
EIVLTQSPGTLSLSPGERATLSCRASQSVASSYLAWYQQKPGQAPRLLIYGASSRATGIPDRFSGSGSGTDFTLTISRLEPEDFAVYYCQQYGSSQLTFGQGTKVEIK

SEQ ID NO: 98 - protein, 118 aa, Artificial Sequence
  Synthetic
QVQLVESGGGVVQPGRSLRLSCAASGFTFRDYGMHWVRQAPGKGLEWVAVISYHGRNEYYANSVKGRFTISRDNSKNTLYLQMNSLRAEDTAVYYCARGAYGDDFDVWGQGTLVTVSS

SEQ ID NO: 99 - protein, 107 aa, Artificial Sequence
  Synthetic
DIQMTQSPSSLSASVGDRVTITCRASQSISSYLNWYQQKTGKAPKLLIYAASSLQSGVPSRFSGSGSGTDFTLTISSLQPEDFATYYCQQSYSAPYTFGQGTKVEIK

SEQ ID NO: 100 - protein, 127 aa, Artificial Sequence
  Synthetic
EVQLLESGGGLVQPGGSLRLSCAASGFTFSNYAMSWVRQAPGKGLEWVSTISGGGRTTNYADSVKGRFTISRDNSKNTLYLQMNSLRAEDTAVYYCARSTPIPSYYPYIYSYYFDVWGQGTLVTVSS

SEQ ID NO: 101 - protein, 107 aa, Artificial Sequence
  Synthetic
EIVMTQSPATLSVSPGERATLSCRASQSVSNNLAWYQQKPGQAPRLLIYGASTRATGIPARFSGSGSGTEFTLTISSLQSEDFAVYYCQQYGNYPPTFGQGTKVEIK

SEQ ID NO: 102 - protein, 121 aa, Artificial Sequence
  Synthetic
EVQLLESGGGLVQPGGSLRLSCAASGFTFSDYAMSWVRQAPGKGLEWVSSISSSGANTYYADSGKGRFTISRDNSKNILYLQMNSLRAEDTAVYYCARSVVTWVTYAFDYWGQGTLVTVSS

SEQ ID NO: 103 - protein, 111 aa, Artificial Sequence
  Synthetic
QSVLTQPPSVSGAPGQRVTISCTGSSSNIGAGYDVHWYQQLPGTAPKLLIYDNTNRPSGVPDRFSGSKSGTSASLAITGLQAEDEADYYCQSYDSGLSGWVFGGGTKLTVL

SEQ ID NO: 104 - protein, 121 aa, Artificial Sequence
  Synthetic
EVQLLESGGGLVQPGGSLRLSCAASGFTFSNYAMSWVRQAPGKGLEWVSAISATGGATYYADSVKGRFTISRDNSKNTLYLQMNSLRAEDTAVYYCARSVRERYTYYMDYWGQGTLVTVSS

SEQ ID NO: 105 - protein, 111 aa, Artificial Sequence
  Synthetic
QSVLTQPPSVSGAPGQRVTISCTGSSSNIGAGYDVHWYQQLPGTAPKLLIYDNTNRPSGVPDRFSGSKSGTSASLAITGLQAEDEADYYCQSYDVSLGVWVFGGGTKLTVL

SEQ ID NO: 106 - protein, 118 aa, Artificial Sequence
  Synthetic
QVQLVQSGAEVKKPGSSVKVSCKASGGTFSNYYISWVRQAPGQGLEWMGGIIPVFGVAHYAQKFQGRVTITADESTSTAYMELSSLRSEDTAVYYCARGYPGEYFDYWGQGTLVTVSS

SEQ ID NO: 107 - protein, 109 aa, Artificial Sequence
  Synthetic
EIVLTQSPGTLSLSPGERATLSCRASQSVSSSYLAWYQQKPGQAPRLLIYAASSRATGIPDRFSGSGSGTDFTLTISRLEPEDFAVYYCQHYGGSPGYTFGQGTKVEIK

SEQ ID NO: 108 - protein, 120 aa, Artificial Sequence
  Synthetic
EVQLLESGGGLVQPGGSLRLSCAASGFTFGSYAMSWVRQAPGKGLEWVSSIYAGGGRTYYADSVKGRFTISRDNSKNTLYLQMNSLRAEDTAVYYCARWGGDGFYAMDYWGQGTLVTVSS

SEQ ID NO: 109 - protein, 107 aa, Artificial Sequence
  Synthetic
DIQMTQSPSSLSASVGDRVTITCRASQTISNYLNWYQQKPGKAPKLLIYGASSLQSGVPSRFSGSGSGTDFTLTISSLQPEDFATYYCQQSFTFPLTFGQGTKVEIK

SEQ ID NO: 110 - protein, 121 aa, Artificial Sequence
  Synthetic
EVQLLESGGGLVQPGGSLRLSCAASGFTFSDYAMSWVRQAPGKGLEWVSTIYSGGVRTYYADSVKGRFTISRDNSKNTLYLQMNSLRAEDTAVYYCARGLVSFRGYAFDYWGQGTLVTVSS

SEQ ID NO: 111 - protein, 107 aa, Artificial Sequence
  Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYTIPPTFGQGTKVEIK

SEQ ID NO: 112 - protein, 121 aa, Artificial Sequence
  Synthetic
EVQLLESGGGLVQPGGSLRLSCAASGFTFRNYAMSWVRQAPGKGLEWVSSISGGGINTYYADSVKGRFTISRDNSKNTLYLQMNSLRAEDTAVYYCARVRSYSGPGYFDYWGQGTLVTVSS

SEQ ID NO: 113 - protein, 107 aa, Artificial Sequence
  Synthetic
EIVMTQSPATLSVSPGDRATLSCRASRSVSSNLAWYQQKPGQAPRLLIYGASTRATGIPARFSGSGSGTEFTLTISSLQSEDFAVYYCQQYYNWPLTFGQGTKVEIK

SEQ ID NO: 114 - protein, 118 aa, Artificial Sequence
  Synthetic
EVQLLESGGGLVQPGGSLRLSCAASGFTFSNYAMSWVRQAPGKGLEWVSVIYGSGARTYYADSVKGRFTISRDNSKNTLYLQMNSLRAEDTAVYYCARISPHYDFDVWGQGTLVTVSS

SEQ ID NO: 115 - protein, 107 aa, Artificial Sequence
  Synthetic
DIQMTQSPSSLSASVGDRVTITCRASQTISKYLNWYQQKPGKAPKLLIYAASSLQSGVPSRFSGSGSGTDFTLTISSLQPEDFATYYCQQSYSYPITFGQGTKVEIK

SEQ ID NO: 116 - protein, 120 aa, Artificial Sequence
  Synthetic
EVQLLESGGGLVQPGGSLRLSCAASGFTFSNYAMSWVRQAPGKGLEWVSAISGGGSRTYYADSVKGRFTISRDNSKNTLYLQMNSLRAEDTAVYYCAREARVLGYSLDYWGQGTLVTVSS

SEQ ID NO: 117 - protein, 107 aa, Artificial Sequence
  Synthetic
DIQMTQSPSSLSASVGDRVTITCRASQRIGKYLNWYQQKPGKAPKLLIYAASSLQSGVPSRFSGSGSGTDFTLTISSLQPEDFATYYCQQSYSYPLTFGQGTKVEIK

SEQ ID NO: 118 - protein, 119 aa, Artificial Sequence
  Synthetic
EVQLLESGGGLVQPGGSLRLSCAASGFTFSNYAMSWVRQAPGKGLEWVSAIYGSGGRTYYADSVKGRFTISRDNSKNTLYLQMNSLRAEDTAVYYCARLRYGFYQFDYWGQGTLVTVSS

SEQ ID NO: 119 - protein, 107 aa, Artificial Sequence; identical to SEQ ID NO: 75
  Synthetic

SEQ ID NO: 120 - protein, 120 aa, Artificial Sequence
  Synthetic
EVQLLESGGGLVQPGGSLRLSCAASGFTFTDYAMSWVRQAPGKGLEWVSTIYSSGHRTYYADSVKGRFTISRDNSKNTLYLQMNSLRAEDTAVYYCARLYPLHSYAFDHWGQGTLVTVSS

SEQ ID NO: 121 - protein, 107 aa, Artificial Sequence
  Synthetic
DIQMTQSHSSLSASVGDRVTITCRASQSISRYLNWYQQKPGKAPKLLIYAASSLQSGVPSRFSGSGSGTDFTLTISSLQPEDFATYYCQQSYSVPLTFGQGTKVEIK

SEQ ID NO: 122 - protein, 125 aa, Artificial Sequence
  Synthetic
EVQLLESGGGLVQPGGSLRLSCAASGFTFKSYAMSWVRQAPGKGLEWVSLISSSGENTYYADSVKGRFTISRDNSKNTLYLQMNSLRAEDTAVYYCARVRFGYGSWRYRKYMDVWGQGTLVTVSS

SEQ ID NO: 123 - protein, 107 aa, Artificial Sequence
  Synthetic
DIQMTQSPSSLSASVGDRVTITCRASQSIATYLNWYQQKPGKAPKLLIYGASSLQSGVPSRFSGSGSGTDFTLTISSLQPEDFATYYCQQSYSTPLTFGQGTKVEIK

SEQ ID NO: 124 - protein, 120 aa, Artificial Sequence
  Synthetic
EVQLLESGGGLVQPGGSLRLSCAASGFTFSSYAMSWVRQAPGKGLEWVSTISASGATYYADSVKGRFTISRDNSKNTLYLQMNSLRAEDTAVYYCARGPYSYYYYSFDYWGQGTLVTVSS

SEQ ID NO: 125 - protein, 107 aa, Artificial Sequence
  Synthetic
DIQMTQSPSSLSASVGDRVTITCRASQTISTYLNWYQQKPGKAPKLLIYAASSLQSGVPSRFSGSGSGTDFTLTISSLQPEDFATYYCQQSYRYPLTFGQGTKVEIK

SEQ ID NO: 126 - protein, 118 aa, Artificial Sequence
  Synthetic
EVQLLESGGGLVQPGGSLRLSCAASGFTFSSYAMSWVRQAPGKGLEWVSTIYVGGHRTYYADSVKGRFTISRDNSKNTLYLQMNSLRAEDTAVYYCARVGWSWGFDPWGQGTLVTVSS

SEQ ID NO: 127 - protein, 107 aa, Artificial Sequence
  Synthetic
DIQMTQSPSSLSASVGDRVTITCRASQTISKYLNWYQQKPGKAPKLLIYSASSLQSGVPSRFSGSGSGTDFTLTISSLQPEDFATYYCQQSYTYPLTFGQGTKVEIK

SEQ ID NO: 128 - protein, 124 aa, Artificial Sequence
  Synthetic
EVQLVESGGGLVKPGGSLRLSCAASGFTFTNAWMSWVRQAPGKGLEWVGRIKSITEGGTTDYAAPVKGRFTISRDDSKNTLYLQMNSLKTEDTAVYYCARVWHTDDYYEGFDVWGQGTLVTVSS

SEQ ID NO: 129 - protein, 108 aa, Artificial Sequence
  Synthetic
EIVLTQSPGTLSLSPGERATLSCRASQSVSGSYLAWYQQKPGQAPRLLIYDASSRATGIPDRFSGSGSGTDFTLTISRLEPEDFAVYYCQQYGTTPQTFGQGTKVEIK

SEQ ID NO: 130 - protein, 120 aa, Artificial Sequence
  Synthetic
EVQLLESGGGLVQPGGSLRLSCAASGFTFRSYAMSWVRQAPGKGLEWVSTIYGSGARTYYADSVKGRFTISRDNSKNTLYLQMNSLRAEDTAVYYCARWGGDGFYAMDYWGQGTLVTVSS

SEQ ID NO: 131 - protein, 107 aa, Artificial Sequence
  Synthetic
DIQMTQSPSSLSASVGDRVTITCRASQSIYTYLNWYQQKPGKAPKLLIYAASSLQSGVPSRFSGSGSGTDFTLTISSLQPEDFATYYCQQSYSFPLTFGQGTKVEIK

SEQ ID NO: 132 - protein, 118 aa, Artificial Sequence
  Synthetic
EVQLLESGGGLVQPGGSLRLSCAASGFTFSSYAMSWVRQAPGKGLEWVSGISTSGGTTYYADSVKGRFTISRDNSKNTLYLQMNSLRAEDTAVYYCARVVGRAVFDIWGQGTLVTVSS

SEQ ID NO: 133 - protein, 108 aa, Artificial Sequence
  Synthetic
EIVLTQSPGTLSLSPGERATLSCRASQSVSSTYLAWYQQKPGQAPRLLIYAASSRATGIPDRFSGSGSGTDFTLTISRLEPEDFAVYYCQQYAALPITFGQGTKVEIK

SEQ ID NO: 134 - protein, 120 aa, Artificial Sequence
  Synthetic
QVQLVQSGAEVKKPGSSVKVSCKASGGTFSDEAIHWVRQAPGQGLEWMGRIIPVLGIASYAQKFQGRVTITADESTSTAYMELSSLRSEDTAVYYCARGLSRKAFYFDYWGQGTLVTVSS

SEQ ID NO: 135 - protein, 107 aa, Artificial Sequence
  Synthetic
DIQMTQSPSSLSASVGDRVTITCRASQTIGNYLNWYQQKPGKAPKLLIYVASSLQSGVPSRFSGSGSGTDFTLTISSLQPEDFATYYCQQSYRYPYTFGQGTKVEIK

SEQ ID NO: 136 - protein, 126 aa, Artificial Sequence
  Synthetic
EVQLLESGGGLVQPGGSLRLSCAASGFTFSNYAMSWVRQAPGKGLEWVSTIYRGAGTYYADSVKGRFTISRDNSKNTLYLQMNSLRAEDTAVYYCARDASWYGPFSYYYYYGFDVWGQGTLVTVSS

SEQ ID NO: 137 - protein, 107 aa, Artificial Sequence
  Synthetic
EIVMTQSPATLSVSPGERATLSCRASQSVYTNLAWYQQKPGQAPRLLIYDASTRATGIPARFSGSGSGTEFTLTISSLQSEDFAVYYCQQYNDWPLTFGQGTKVEIK

SEQ ID NO: 138 - protein, 124 aa, Artificial Sequence
  Synthetic
EVQLLESGGGLVQPGGSLRLSCAASGFTFSDYAMSWVRQAPGKGLEWVSIITESGVNTYYADSVKGRFTISRDNSKNTLYLQMNSLRAEDTAVYYCARHPSVGSASRSYYFDVWGQGTLVTVSS

SEQ ID NO: 139 - protein, 108 aa, Artificial Sequence
  Synthetic
EIVLTQSPGTLSLSPGERATLSCRASQSVGSSYLAWYQQKPGQAPRLLIYAASSRATGIPDRFSGSGSGTDFTLTISRLEPEDFAVYYCQQYYSAPLTFGQGTKVEIK

SEQ ID NO: 140 - protein, 127 aa, Artificial Sequence
  Synthetic
EVQLLESGGGLVQPGGSLRLSCAASGFTFTDYAMSWVRQAPGKGLEWVSGISGSGGVGTYYADSVKGRFTISRDNSKNTLYLQMNSLRAEDTAVYYCARGEYGSSIAYSYYYGFDVWGQGTLVTVSS

SEQ ID NO: 141 - protein, 107 aa, Artificial Sequence
  Synthetic
EIVMTQSPATLSVSPGERATLSCRASQSVYNNLAWYQQKPGQAPRLLIYDASTRATGIPARFSGSGSGTEFTLTISSLQSEDFAVYYCQQYNDWPYTFGQGTKVEIK

SEQ ID NO: 142 - protein, 120 aa, Artificial Sequence; identical to SEQ ID NO: 116
  Synthetic

SEQ ID NO: 143 - protein, 107 aa, Artificial Sequence; identical to SEQ ID NO: 117
  Synthetic

SEQ ID NO: 144 - protein, 119 aa, Artificial Sequence
  Synthetic
EVQLLESGGGLVQPGGSLRLSCAASGFTFSGYAMSWVRQAPGKGLEWVSAIRGGGGTYYADSVKGRFTISRDNSKNTLYLQMNSLRAEDTAVYYCARVGPLRLYGFDYWGQGTLVTVSS

SEQ ID NO: 145 - protein, 107 aa, Artificial Sequence
  Synthetic
DIQMTQSPSSLSASVGDRVTITCRASQSISNYLNWYQQKPGKAPKLLIYAASSLQSGVPSRFSGSGSGTDFTLTISSLQPEDFATYYCQQSYSYPLTFGQGTKVEIK

SEQ ID NO: 146 - protein, 126 aa, Artificial Sequence
  Synthetic
EVQLLESGGGLVQPGGSLRLSCAASGFTFSSYAMSWVRQAPGKGLEWVSVISGSGGNTYYADSVKGRFTISRDNSKNTLYLQMNSLRAEDTAVYYCAREVRHDSYYYYYYSGMDVWGQGTLVTVSS

SEQ ID NO: 147 - protein, 107 aa, Artificial Sequence
  Synthetic
EIVMTQSPATLSVSPGERATLSCRASQSVYNNLAWYQQKPGQAPRLLIYDASTRATGIPARFSGSGSGTEFTLTISSLQSEDFAVYYCQQYSDYPFTFGQGTKVEIK

SEQ ID NO: 148 - protein, 125 aa, Artificial Sequence
  Synthetic
QVQLVQSGAEVKKPGSSVKVSCKASGGTFSNYAINWVRQAPGQGLEWMGRIIPTLGIANYAQKFQGRVTITADESTSTAYMELSSLRSEDTAVYYCARAAYFAGPAARYGYFDIWGQGTLVTVSS

SEQ ID NO: 149 - protein, 108 aa, Artificial Sequence
  Synthetic
EIVLTQSPGTLSLSPGERATLSCRASQSVRSNYLAWYQQKPGQAPRLLIYDASSRATGIPDRFSGSGSGTDFTLTISRLEPEDFAVYYCQQYAYAPWTFGQGTKVEIK

SEQ ID NO: 150 - protein, 118 aa, Artificial Sequence
  Synthetic
EVQLLESGGGLVQPGGSLRLSCAASGFTFRGYAMSWVRQAPGKGLEWVSSISVSGAGTYYADSVKGRFTISRDNSKNTLYLQMNSLRAEDTAVYYCARYVRGVSFDVWGQGTLVTVSS

SEQ ID NO: 151 - protein, 109 aa, Artificial Sequence; identical to SEQ ID NO: 61
  Synthetic

SEQ ID NO: 152 - protein, 119 aa, Artificial Sequence; identical to SEQ ID NO: 118
  Synthetic

SEQ ID NO: 153 - protein, 107 aa, Artificial Sequence; identical to SEQ ID NO: 75
  Synthetic

SEQ ID NO: 154 - protein, 121 aa, Artificial Sequence; identical to SEQ ID NO: 110
  Synthetic

SEQ ID NO: 155 - protein, 107 aa, Artificial Sequence; identical to SEQ ID NO: 111
  Synthetic

SEQ ID NO: 156 - protein, 118 aa, Artificial Sequence; identical to SEQ ID NO: 70
  Synthetic

SEQ ID NO: 157 - protein, 107 aa, Artificial Sequence
  Synthetic
DIQMTQSPSSLSASVGDRMTITCRASQSISNYLNWYQQKPGKAPKLLIYAASSLQSGVPSRFSGSGSGTDFTLTISSLQPEDFATYYCQQSYAPPYTFGQGTKVEIK

SEQ ID NO: 158 - protein, 119 aa, Artificial Sequence
  Synthetic
EVQLLESGGGLVQPGGSLRLSCAASGFTFSSYAMSWVRQAPGKGLEWVSGISESGGGTYYADSVKGRFTISRDNSKNTLYLQMNSLRAEDTAVYYCARLVPWQLAFDVWGQGTLVTVSS

SEQ ID NO: 159 - protein, 107 aa, Artificial Sequence
  Synthetic
EIVMTQSPATLSVSPGERATLSCRASQSVYNNLAWYQQKPGQAPRLLIYDASTRATGIPARFSGSGSGTEFTLTISSLQSEDFAVYYCQQYYYWPITFGQGTKVEIK

SEQ ID NO: 160 - protein, 126 aa, Artificial Sequence; identical to SEQ ID NO: 146
  Synthetic

SEQ ID NO: 161 - protein, 107 aa, Artificial Sequence; identical to SEQ ID NO: 147
  Synthetic

SEQ ID NO: 162 - protein, 126 aa, Artificial Sequence
  Synthetic
QVQLVQSGAEVKKPGSSVKVSCKASGGTFSSEAISWVRQAPGQGLEWMGRIIPISGRPNYAQKFQGRVTITADESTSTAYMELSSLRSEDTAVYYCAREASYYVAGYYRYYGFDVWGQGTLVTVSS

SEQ ID NO: 163 - protein, 107 aa, Artificial Sequence
  Synthetic
EIVMTQSPATLSVSPGERATLSCRASRSLSNNLAWYQQKPGQAPRLLIYDASTRATGIPARFSGSGSGTEFTLTISSLQSEDFAVYYCQQYNAWPYTFGQGTKVEIK

SEQ ID NO: 164 - protein, 126 aa, Artificial Sequence; identical to SEQ ID NO: 146
  Synthetic

SEQ ID NO: 165 - protein, 107 aa, Artificial Sequence; identical to SEQ ID NO: 147
  Synthetic

SEQ ID NO: 166 - protein, 126 aa, Artificial Sequence
  Synthetic
QVQLVESGGGLVKPGGSLRLSCAASGFTFSNAWMSWVRQAPGKGLEWVGRIKSKIEGGTTDYAPPVKGRFTISRDDSKNTLYLQMNSLKTEDTAVYYCARDPTWYVSGHYYGFDVWGQGTLVTVSS

SEQ ID NO: 167 - protein, 107 aa, Artificial Sequence
  Synthetic
DIQMTQSPSSLSASVGDRVTITCRASQSIANYLNWYQQKPGKAPKLLIYAASSLQSGVPSRFSGSGSGTDFTLTISSLQPEDFATYYCQQYFSTPPTFGQGTKVEIK

SEQ ID NO: 168 - protein, 121 aa, Artificial Sequence
  Synthetic
EVQLLESGGGLVQPGGSLRLSCAASGFTFGNYAMSWVRQAPGKGLEWVSTISAGGSRTYYADSVKGRFTISRDNSKNTLYLQMNSLRAEDTAVYYCARGWYPILYYAFDYWGQGTLVTVSS

SEQ ID NO: 169 - protein, 107 aa, Artificial Sequence
  Synthetic
DIQMTQSPSSLSASVGDRVTITCRASQSIGNYLNWYQQKPGKAPKLLIYSASSLQSGVPSRFSGSGSGTDFTLTISSLQPEDFATYYCQQTYTIPLTFGQGTKVEIK

SEQ ID NO: 170 - protein, 122 aa, Artificial Sequence
  Synthetic
EVQLLESGGGLVQPGGSLRLSCAASGFTFSSYAMSWVRQAPGKGLEWVSTIYRSGGRTYYADSVKGRFTISRDNSKNTLYLQMNSLRAEDTAVYYCARGAYPLGSYYGFDPWGQGTLVTVSS

SEQ ID NO: 171 - protein, 107 aa, Artificial Sequence
  Synthetic
DIQMTQSPSSLSASVGDRVTITCRASQSIATYLNWYQQKPGKAPKLLIYAASSLQSGVPSRFSGSGSGTDFTLTISSLQPEDFATYYCQQSYTYPVTFGQGTKVEIK

SEQ ID NO: 172 - protein, 121 aa, Artificial Sequence
  Synthetic
EVQLLESGGGLVQPGGSLRLSCAASGFTFSNYAMSWVRQAPGKGLEWVSTIYGSGVRTYYADGVKGRFTISRDNSKNTLYLQMNSLRAEDTAVYYCARGWIPHSFYAFDYWGQGTLVTVSS

SEQ ID NO: 173 - protein, 107 aa, Artificial Sequence
  Synthetic
DIQMTQSPSSLSASVGDRVTITCRASQSISSYLNWYQQKPGKAPKLLIYGASSLQSGVPSRFSGSGSGTDFTLTISSLQPEDFATYYCQQSFTAPVTFGQGTKVEIK

SEQ ID NO: 174 - protein, 121 aa, Artificial Sequence
  Synthetic
EVQLLESGGGLVQPGGSLRLSCAASGFTFTNYAMSWVRQAPGKGLEWVSSISGSGISTYYADSVKGRFTISRDNSKNTLYLQMNSLRAEDTAVYYCARGPGPGYWYAFDVWGQGTLVTVSS

SEQ ID NO: 175 - protein, 107 aa, Artificial Sequence
  Synthetic
DIQMTQSPSSLSASVGDRVTITCRASQTISRYLNWYQQKPGKAPKLLIYAASSLQSGVPSRFSGSGSGTDFTLTISSLQPEDFATYYCQQSYTYPITFGQGTKVEIK

SEQ ID NO: 176 - protein, 120 aa, Artificial Sequence
  Synthetic
QVQLVQSGAEVKKPGSSVKVSCKASGGTFSDEAIHWVRQAPGQGLEWMGRIIPVLGIASYAQKFQGRVTITADESTSTAYMELSSLRSEDTAVYYCARGLSRRAFYFDYWGQGTLVTVSS

SEQ ID NO: 177 - protein, 107 aa, Artificial Sequence; identical to SEQ ID NO: 135
  Synthetic

SEQ ID NO: 178 - protein, 121 aa, Artificial Sequence; identical to SEQ ID NO: 174
  Synthetic

SEQ ID NO: 179 - protein, 107 aa, Artificial Sequence
  Synthetic
DIQMTQSPSSLSASVGDRVTITCRASQTISRYLNWYQQKPGKAPKLLIYSASSLQSGVPSRFSGSGSGTDFTLTISSLQPEDFATYYCQQSYTYPITFGQGTKVEIK

SEQ ID NO: 180 - protein, 121 aa, Artificial Sequence
  Synthetic
EVQLLESGGGLVQPGGSLRLSCAASGFTFSDYAMSWVRQAPGKGLEWVSTIYSGGVRTYYADSVKGRFTISRDNSKNTLYLQMNSLRAEDTAVYYCARGLVSFKGYAFDYWGQGTLVTVSS

SEQ ID NO: 181 - protein, 107 aa, Artificial Sequence; identical to SEQ ID NO: 111
  Synthetic

SEQ ID NO: 182 - protein, 118 aa, Artificial Sequence
  Synthetic
EVQLLESGGGLVQPGGSLRLSCAASGFTFSSYAMSWVRQAPGKGLEWVSAIYGAGGKTYYADSVKGRFTISRDNSKNTLYLQMNSLRAEDTAVYYCARISHGWGFDVWGQGTLVTVSS

SEQ ID NO: 183 - protein, 107 aa, Artificial Sequence
  Synthetic
DIQMTQSPSSLSASVGDRVTITCRASQDISNYLNWYQQKPGKAPKLLIYAASSLQSGVPSRFSGSGSGTDFTLTISSLQPEDFATYYCQQSYTYPLTFGQGTKVEIK

SEQ ID NO: 184 - protein, 120 aa, Artificial Sequence
  Synthetic
EVQLLESGGGLVQPGGSLRLSCAASGFTFSNYAMSWVRQAPGKGLEWVSVIYGSGTRTYYADSVKGRFTISRDNSKNTLYLQMNSLRAEDTAVYYCARVASSYHYAFDYWGQGTLVTVSS

SEQ ID NO: 185 - protein, 107 aa, Artificial Sequence
  Synthetic
DIQMTQSPSSLSASVGDRVTITCRASQSISTYLNWYQQKPGKAPKLLIYAASSLQSGVSSRFSGSGSGTDFTLTISSLQPEDFATYYCQQSYTFPLTFGQGTKVEIK

SEQ ID NO: 186 - protein, 120 aa, Artificial Sequence
  Synthetic
EVQLLESGGGLVQPGGSLRLSCAASGFTFSNYAMSWVRQAPGKGLEWVSVIYGSGGRTYYADSVKGRFTISRDNSKNTLYLQMNSLRAEDTAVYYCARVEDTSYYGMDYWGQGTLVTVSS

SEQ ID NO: 187 - protein, 107 aa, Artificial Sequence
  Synthetic
DIQMTQSPSSLSASVGDRVTITCRASQTIGIYLNWYQQKPGKAPTLLIYAASSLQSGVPSRFSGSGSGTDFTLTISSLQPEDFATYYCQQSYTYPLTFGQGTKVEIK

SEQ ID NO: 188 - protein, 119 aa, Artificial Sequence
  Synthetic
EVQLLESGGGLVQPGGSLRLSCAASGFTFSNYAMSWVRQAPGKGLEWVSAIYGSGGRTYYADSVKGRFTISRDNSKNTLYLQMNSLRAEDTAVYYCARLEAGFYSLDIWGQGTLVTVSS

SEQ ID NO: 189 - protein, 107 aa, Artificial Sequence
  Synthetic
DIQMTQSPSSLSASVGDRVTITCRASQTIGTYLNWYQQKPGKAPKLLIYAASSLQSGVPSRFSGSGSGTDFTLTISSLQPEDFATYYCQQSYSYPLTFGQGTKVEIK

SEQ ID NO: 190 - protein, 119 aa, Artificial Sequence
  Synthetic
EVQLLESGGGLVQPGGSLKLSCAASGFTFGSYAMSWVRQAPGKGLEWVSVISSGGSYTYYADSVKGRFTISRDNSKNTLYLQMNSLRAEDTAVYYCARVSGVVGALDIWGQGTLVTVSS

SEQ ID NO: 191 - protein, 107 aa, Artificial Sequence
  Synthetic
EIVMTQSPATLSVSPGERATLSCRASQSVSNNLAWYQQKPGQAPRLLIYGASTRATGIPARFSGSGSGTEFTLTISSLQSEDFAVYYCQQYNNWPYTFGQGTKVEIK

SEQ ID NO: 192 - protein, 120 aa, Artificial Sequence
  Synthetic
EVQLLESGGGLVQPGGSLRLSCAASGFTFSNYAMSWVRQAPGKGLEWVSTIYSSGSRTYYADSVKGRFTISRDNSKNTLYLQMNSLRAEDTAVYYCARWGGDGFYAMDYWGQGTLVTVSS

SEQ ID NO: 193 - protein, 107 aa, Artificial Sequence
  Synthetic
DIQMTQSPSSLSASVGDRVTITCRASQSISTYLNWYQQKPGKAPKLLIYAASSLQSGVPSRFSGSGSGTDFTLTISSLQPEDFATYYCQQSYTVPLTFGQGTKVEIK

SEQ ID NO: 194 - protein, 121 aa, Artificial Sequence; identical to SEQ ID NO: 180
  Synthetic

SEQ ID NO: 195 - protein, 107 aa, Artificial Sequence; identical to SEQ ID NO: 111
  Synthetic

SEQ ID NO: 196 - protein, 118 aa, Artificial Sequence
  Synthetic
EVQLLESGGGLVQPGGSLRLSCAASGFTFSSYAMSWVRQAPGKGLEWVSAIYGSGGDTYYADSVKGRFTISRDNSKNTLYLQMNSLRAEDTAVYYCARTEYDGYFDVWGQGTLVTVSS

SEQ ID NO: 197 - protein, 107 aa, Artificial Sequence
  Synthetic
DIQMTQSPSSLSASVGDRVTITCRASQDIYNYLNWYQQKPGKAPKLLIYGASSLQSGVPSRFSGSGSGTDFTLTISSLQPEDFATYYCQQSDGFPPTFGQGTKVEIK

SEQ ID NO: 198 - protein, 119 aa, Artificial Sequence
  Synthetic
EVQLLESGGGLVQPGGSLRLSCAASGFTFTDYAMSWVRQAPGKGLEWVSVIYGSGGRTYYADSVKGRFTISRDNSKNTLYLQMNSLRAEDTAVYYCARVEDSHYSFDVWGQGTLVTVSS

SEQ ID NO: 199 - protein, 107 aa, Artificial Sequence
  Synthetic
DIQMTQSPSSLSASVGDRVTITCRASQSISNYLNWYQQKPGKAPKLLIYAASSLQSGVPSRFSGSGSGTDFTLTISSLQPEDFATYYCQQSYTVPWTFGQGTKVEIK

SEQ ID NO: 200 - protein, 126 aa, Artificial Sequence
  Synthetic
QVQLVESGGGVVQPGRSLRLSCAASGFPFSDYAMHWLRQAPGKGLEWVAVISYDGNIEYYANSVKGRFTISRDNSKNTLYLQMNSLRAEDTAVYYCARDRIGSSTYYGYYNGFDVWGQGTLVTVSS

SEQ ID NO: 201 - protein, 107 aa, Artificial Sequence
  Synthetic
EIVMTQSPATLSVSPGERATLSCRASQSVSSNLAWYQQKPGQAPRLLIYGASTRATGIPARFSGSGSGTEFTLTISSLQSEDFAVYYCQQYNDYPLTFGQGTKVEIK

SEQ ID NO: 202 - protein, 117 aa, Artificial Sequence
  Synthetic
EVQLLESGGGLVQPGGSLRLSCAASGFTFSNYAMSWVRQAPGKGLEWVSAIKGSGGGTYYADSVKGRFTISRDNSKNTLYLQMNSLRAEDTAVYYCARGGYLWLDPWGQGTLVTVSS

SEQ ID NO: 203 - protein, 107 aa, Artificial Sequence
  Synthetic
EIVMTQSPATLSVSPGERATLSCRASQSISSNLAWYQQKPGQAPRLLIYDASTRATGIPARFSGSGSGTEFTLTISSLQSEDFAVYYCQQYYNWPLTFGQGTKVEIK

SEQ ID NO: 204 - protein, 125 aa, Artificial Sequence
  Synthetic
EVQLLESGGGLVQPGGSLRLSCAASGFTFSNYAMSWVRQAPGKGLEWVSAISGSGTYTYYADSVKGRFTISRDNSKNTLYLQMNSLRAEDTAVYYCARVRISGYGPGYFGGFDVWGQGTLVTVSS

SEQ ID NO: 205 - protein, 107 aa, Artificial Sequence
  Synthetic
EIVMTQSPATLSVSPGERATLSCRASQSVYSNLAWYQQKPGQAPRLLIYGASTRATGIPARFSGSGSGTEFTLTISSLQSEDFAVYYCQQYSNWPLTFGQGTKVEIK

SEQ ID NO: 206 - protein, 125 aa, Artificial Sequence
  Synthetic
EVQLLESGGGLVQPGGSLRLSCAASGFTFSRYAMSWVRQAPGKGLEWVSIISGTGGNTYYADSVKGRFTISRDNSKNTLYLQMNSLRAEDTAVYYCARIDTFAYYYSLYRAFDLWGQGTLVTVSS

SEQ ID NO: 207 - protein, 107 aa, Artificial Sequence
  Synthetic
EIVMTQSPATLSVSPGERATLSCRASQSVGGNLAWYQQKPGQAPRLLIYDASTRATGIPARFSGSGSGTEFTLTISSLQSEDFAVYYCQQYNDWPLTFGQGTKVEIK

SEQ ID NO: 208 - protein, 124 aa, Artificial Sequence
  Synthetic
EVQLLESGGGLVQPGGSLRLSCAASGFTFSDYAMSWVRQAPGKGLEWVSSISEGGGSTYYADSVKGRFTISRDNSKNTLYLQMNSLRAEDTAVYYCARTKRYFAGGSYYWMDVWGQGTLVTVSS

SEQ ID NO: 209 - protein, 107 aa, Artificial Sequence
  Synthetic
DIQMTQSPSSLSASVGDRVTITCRASQRISSYLNWYQQKPGKAPKLLIYAASSLQSGVPSRFSGSGSGTDFALTISSLQPEDFATYYCQQSYSYPITFGQGTKVEIK

SEQ ID NO: 210 - protein, 120 aa, Artificial Sequence
  Synthetic
EVQLLESGGGLVQPGGSLRLSCAASGFTFSSYAMSWVRQAPGKGLEWVSVISGGSGHTTYYADSVKGRFTISRDNSKNTLYLQMNSLRAEDTAVYYCARHGGGRFAMDVWGQGTLVTVSS

SEQ ID NO: 211 - protein, 108 aa, Artificial Sequence
  Synthetic
EIVLTQSPGTLSLSPGERATLSCRASQSVASPYLAWYQQKPGQAPRLLIYGASSRATGIPDRFSGSGSGTDFTLTISRLEPEDFAVYYCQQYYESPITFGQGTKVEIK

SEQ ID NO: 212 - protein, 127 aa, Artificial Sequence
  Synthetic
EVQLLESGGGLVQPGGSLRLSCAASGFTFSTYAMSWVRQAPGKGLEWVSSISGSGGKTYYADSVKGRFTISRDNSKNTLYLQMNSLRAEDTAVYYCARDIYGITYDGYTYYDGMDVWGQGTLVTVSS

SEQ ID NO: 213 - protein, 107 aa, Artificial Sequence
  Synthetic
DIQMTQSPSSLSASVGDRVTITCRASQSIYNYLNWHQQKPGKAPKLLIYAASSLQSGVPSRFSGSGSGTDFTLTISSLQPEDFATYYCQQSDSPPWTFGQGTKVEIK

SEQ ID NO: 214 - protein, 118 aa, Artificial Sequence; identical to SEQ ID NO: 106
  Synthetic

SEQ ID NO: 215 - protein, 109 aa, Artificial Sequence; identical to SEQ ID NO: 107
  Synthetic

SEQ ID NO: 216 - protein, 123 aa, Artificial Sequence
  Synthetic
EVQLLESGGGLVQPGGSLRLSCAASGFTFSNYAMSWVRQAPGKGLEWVSTISGSGANTYYADSVKGRFTISRDNSKNTLYLQMNSLRAEDTAVYYCARHHDIAPSWIYYFDYWGQGTLVTVSS

SEQ ID NO: 217 - protein, 108 aa, Artificial Sequence
  Synthetic
EIVLTQSPGTLSLSPGERATLSCRARQSVPSNYLAWYQQKPGQAPRLLIYGASSRATGIPDRFSGSGSGTDFTLTISRLEPEDFAVYYCQQYYSSPLTFGQGTKVEIK

SEQ ID NO: 218 - protein, 121 aa, Artificial Sequence
  Synthetic
EVQLLESGGGLVQPGGSLRLSCAASGFTFPDYAMSWVRQAPGKGLEWVSTIYAGGGRTYYADSVKGRFTISRDNSKNTLYLQMNSLRAEDTAVYYCARGLTRHYGYSFDHWGQGTLVTVSS

SEQ ID NO: 219 - protein, 107 aa, Artificial Sequence
  Synthetic
DIQMTQSPSSLSASVGDRVTITCRASQTISSYLNWYQQKPGKAPKLLIYSASSLQSGVPSRFSGSGSGTDFTLTISSLQPEDFATYYCQQSYTFPLTFGQGTKVEIK

SEQ ID NO: 220 - protein, 120 aa, Artificial Sequence
  Synthetic
EVQLLESGGGLVQPGGSLRLSCAASGFTFSNYAMSWVRQAPGKGLEWVSSIYGSGHRTYYADSVKGRFTISRDNSKNTLYLQMNSLRAEDTAVYYCARVASSAEYAFDHWGQGTLVTVSS

SEQ ID NO: 221 - protein, 107 aa, Artificial Sequence
  Synthetic
DIQMTQSPSSLSASVGDRVTITCRASQSISNYLNWYQQKSGKAPKLLIYAASSLQSGVPSRFSGSGSGTDFTLTISSLQPEDFATYYCQQSYTVPLTFGQGTKVEIK

SEQ ID NO: 222 - protein, 126 aa, Artificial Sequence; identical to SEQ ID NO: 46
  Synthetic

SEQ ID NO: 223 - protein, 109 aa, Artificial Sequence
  Synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQYSSSPTYTFGQGTKVEIK

SEQ ID NO: 224 - protein, 5 aa, Artificial Sequence
  Synthetic
SYAMS

SEQ ID NO: 225 - protein, 5 aa, Artificial Sequence
  Synthetic
SYGMH

SEQ ID NO: 226 - protein, 5 aa, Artificial Sequence
  Synthetic
NYAMS

SEQ ID NO: 227 - protein, 5 aa, Artificial Sequence
  Synthetic
NYGMH

SEQ ID NO: 228 - protein, 5 aa, Artificial Sequence
  Synthetic
DYDMH

SEQ ID NO: 229 - protein, 5 aa, Artificial Sequence
  Synthetic
DYAMS

SEQ ID NO: 230 - protein, 7 aa, Artificial Sequence
  Synthetic
SSNYYWG

SEQ ID NO: 231 - protein, 5 aa, Artificial Sequence
  Synthetic
DYGMH

SEQ ID NO: 232 - protein, 5 aa, Artificial Sequence
  Synthetic
TYAMS

SEQ ID NO: 233 - protein, 5 aa, Artificial Sequence
  Synthetic
SYGIH

SEQ ID NO: 234 - protein, 5 aa, Artificial Sequence
  Synthetic
GYAMS

SEQ ID NO: 235 - protein, 5 aa, Artificial Sequence
  Synthetic
TEYIS

SEQ ID NO: 236 - protein, 7 aa, Artificial Sequence
  Synthetic
SSSYYWG

SEQ ID NO: 237 - protein, 5 aa, Artificial Sequence
  Synthetic
RYAMH

SEQ ID NO: 238 - protein, 5 aa, Artificial Sequence
  Synthetic
GYGMH

SEQ ID NO: 239 - protein, 7 aa, Artificial Sequence
  Synthetic
SRGYYWA

SEQ ID NO: 240 - protein, 5 aa, Artificial Sequence
  Synthetic
SYAIN

SEQ ID NO: 241 - protein, 5 aa, Artificial Sequence
  Synthetic
NYYIS

SEQ ID NO: 242 - protein, 5 aa, Artificial Sequence
  Synthetic
NAWMS

SEQ ID NO: 243 - protein, 5 aa, Artificial Sequence
  Synthetic
DEAIH

SEQ ID NO: 244 - protein, 5 aa, Artificial Sequence
  Synthetic
NYAIN

SEQ ID NO: 245 - protein, 5 aa, Artificial Sequence
  Synthetic
SEAIS

SEQ ID NO: 246 - protein, 5 aa, Artificial Sequence
  Synthetic
DYAMH

SEQ ID NO: 247 - protein, 5 aa, Artificial Sequence
  Synthetic
RYAMS

SEQ ID NO: 248 - protein, 17 aa, Artificial Sequence
  Synthetic
AISAGGGYTYYADSVKG

SEQ ID NO: 249 - protein, 17 aa, Artificial Sequence
  Synthetic
VISHSGSNKYYANSVKG

SEQ ID NO: 250 - protein, 15 aa, Artificial Sequence
  Synthetic
TISGGGTYYADSVKG

SEQ ID NO: 251 - protein, 17 aa, Artificial Sequence
  Synthetic
VISYHGRNEYYADSVKG

SEQ ID NO: 252 - protein, 17 aa, Artificial Sequence
  Synthetic
VISHSGSNKYYADSVKG

SEQ ID NO: 253 - protein, 17 aa, Artificial Sequence
  Synthetic
SIYTSGGRTYYADSVKG

SEQ ID NO: 254 - protein, 17 aa, Artificial Sequence
  Synthetic
TIYGRGERTYYADSVKG

SEQ ID NO: 255 - protein, 16 aa, Artificial Sequence
  Synthetic
SVYYTGRTYYNPSLKS

SEQ ID NO: 256 - protein, 17 aa, Artificial Sequence
  Synthetic
VISYEGSNEYYADSVKG

SEQ ID NO: 257 - protein, 17 aa, Artificial Sequence
  Synthetic
SIYGSGGKTYYADSVKG

SEQ ID NO: 258 - protein, 17 aa, Artificial Sequence
  Synthetic
VISFDGSNKYYANSVKG

SEQ ID NO: 259 - protein, 17 aa, Artificial Sequence
  Synthetic
SITGSGEYTNYADSVKG

SEQ ID NO: 260 - protein, 17 aa, Artificial Sequence
  Synthetic
TISGTGGRTYYADSVKG

SEQ ID NO: 261 - protein, 17 aa, Artificial Sequence
  Synthetic
TIYGGGSYTYYADSVKG

SEQ ID NO: 262 - protein, 17 aa, Artificial Sequence
  Synthetic
TIYGSGGRTYYADSVKG

SEQ ID NO: 263 - protein, 17 aa, Artificial Sequence
  Synthetic
AIYGSGSYTYYADSVKG

SEQ ID NO: 264 - protein, 17 aa, Artificial Sequence
  Synthetic
VISGGGTNTYYADSVKG

SEQ ID NO: 265 - protein, 17 aa, Artificial Sequence
  Synthetic
TIYASGATTYYADSVKG

SEQ ID NO: 266 - protein, 17 aa, Artificial Sequence
  Synthetic
TIYGSGSRTYYADSVKG

SEQ ID NO: 267 - protein, 17 aa, Artificial Sequence
  Synthetic
TIYESGGYTYYADSVKG

SEQ ID NO: 268 - protein, 17 aa, Artificial Sequence
  Synthetic
RIVPILGTTNYAQKFQG

SEQ ID NO: 269 - protein, 17 aa, Artificial Sequence
  Synthetic
GISEGGANTYYADSVKG

SEQ ID NO: 270 - protein, 17 aa, Artificial Sequence
  Synthetic
SISVSGAGTYYADSVKG

SEQ ID NO: 271 - protein, 17 aa, Artificial Sequence
  Synthetic
AIGGSGGGTYYADSVKG

SEQ ID NO: 272 - protein, 17 aa, Artificial Sequence
  Synthetic
GITAGGGNTYYADSVKG

SEQ ID NO: 273 - protein, 17 aa, Artificial Sequence
  Synthetic
GISASGGGTYYADSVKG

SEQ ID NO: 274 - protein, 17 aa, Artificial Sequence
  Synthetic
GISVSGGYTYYADSVKG

SEQ ID NO: 275 - protein, 17 aa, Artificial Sequence
  Synthetic
TIYGTGEDTYYADSVKG

SEQ ID NO: 276 - protein, 17 aa, Artificial Sequence
  Synthetic
AISSSGGTTYYADSVKG

SEQ ID NO: 277 - protein, 17 aa, Artificial Sequence
  Synthetic
GISGSGGYTYYADSVKG

SEQ ID NO: 278 - protein, 18 aa, Artificial Sequence
  Synthetic
GISAGGGARTYYADSVKG

SEQ ID NO: 279 - protein, 17 aa, Artificial Sequence
  Synthetic
VIYGSGGRTYYADSVKG

SEQ ID NO: 280 - protein, 17 aa, Artificial Sequence
  Synthetic
VISYDGSRKYYANSVKG

SEQ ID NO: 281 - protein, 17 aa, Artificial Sequence
  Synthetic
RIIPVLGITNYAQKFQG

SEQ ID NO: 282 - protein, 17 aa, Artificial Sequence
  Synthetic
AIYGSGGKTYYADSVKG

SEQ ID NO: 283 - protein, 17 aa, Artificial Sequence
  Synthetic
VIYAGGARTYYADSVKG

SEQ ID NO: 284 - protein, 17 aa, Artificial Sequence
  Synthetic
AISGSGGTTYYADSVKG

SEQ ID NO: 285 - protein, 16 aa, Artificial Sequence
  Synthetic
NIYYTGTTYYNPSLKS

SEQ ID NO: 286 - protein, 17 aa, Artificial Sequence
  Synthetic
AISASGGYTYYADSVKG

SEQ ID NO: 287 - protein, 17 aa, Artificial Sequence
  Synthetic
GISRGGARTYYADSVKG

SEQ ID NO: 288 - protein, 17 aa, Artificial Sequence
  Synthetic
VISHSGSTKYYANSVKG

SEQ ID NO: 289 - protein, 17 aa, Artificial Sequence
  Synthetic
VISHHGSYKYYANSVKG

SEQ ID NO: 290 - protein, 17 aa, Artificial Sequence
  Synthetic
AISRGGGYTYYADSVKG

SEQ ID NO: 291 - protein, 16 aa, Artificial Sequence
  Synthetic
SIYYSGSTYYNPSLKS

SEQ ID NO: 292 - protein, 17 aa, Artificial Sequence
  Synthetic
AISGSGGSTYYADSVKG

SEQ ID NO: 293 - protein, 17 aa, Artificial Sequence
  Synthetic
RIIPLLETADYAQKFQG

SEQ ID NO: 294 - protein, 17 aa, Artificial Sequence
  Synthetic
VISYHGRNEYYANSVKG

SEQ ID NO: 295 - protein, 17 aa, Artificial Sequence
  Synthetic
TISGGGRTTNYADSVKG

SEQ ID NO: 296 - protein, 17 aa, Artificial Sequence
  Synthetic
SISSSGANTYYADSGKG

SEQ ID NO: 297 - protein, 17 aa, Artificial Sequence
  Synthetic
AISATGGATYYADSVKG

SEQ ID NO: 298 - protein, 17 aa, Artificial Sequence
  Synthetic
GIIPVFGVAHYAQKFQG

SEQ ID NO: 299 - protein, 17 aa, Artificial Sequence
  Synthetic
SIYAGGGRTYYADSVKG

SEQ ID NO: 300 - protein, 17 aa, Artificial Sequence
  Synthetic
TIYSGGVRTYYADSVKG

SEQ ID NO: 301 - protein, 17 aa, Artificial Sequence
  Synthetic
SISGGGINTYYADSVKG

SEQ ID NO: 302 - protein, 17 aa, Artificial Sequence
  Synthetic
VIYGSGARTYYADSVKG

SEQ ID NO: 303 - protein, 17 aa, Artificial Sequence
  Synthetic
AISGGGSRTYYADSVKG

SEQ ID NO: 304 - protein, 17 aa, Artificial Sequence
  Synthetic
AIYGSGGRTYYADSVKG

SEQ ID NO: 305 - protein, 17 aa, Artificial Sequence
  Synthetic
TIYSSGHRTYYADSVKG

SEQ ID NO: 306 - protein, 17 aa, Artificial Sequence
  Synthetic
LISSSGENTYYADSVKG

SEQ ID NO: 307 - protein, 16 aa, Artificial Sequence
  Synthetic
TISASGATYYADSVKG

SEQ ID NO: 308 - protein, 17 aa, Artificial Sequence
  Synthetic
TIYVGGHRTYYADSVKG

SEQ ID NO: 309 - protein, 19 aa, Artificial Sequence
  Synthetic
RIKSITEGGTTDYAAPVKG

SEQ ID NO: 310 - protein, 17 aa, Artificial Sequence
  Synthetic
TIYGSGARTYYADSVKG

SEQ ID NO: 311 - protein, 17 aa, Artificial Sequence
  Synthetic
GISTSGGTTYYADSVKG

SEQ ID NO: 312 - protein, 17 aa, Artificial Sequence
  Synthetic
RIIPVLGIASYAQKFQG

SEQ ID NO: 313 - protein, 16 aa, Artificial Sequence
  Synthetic
TIYRGAGTYYADSVKG

SEQ ID NO: 314 - protein, 17 aa, Artificial Sequence
  Synthetic
IITESGVNTYYADSVKG

SEQ ID NO: 315 - protein, 18 aa, Artificial Sequence
  Synthetic
GISGSGGVGTYYADSVKG

SEQ ID NO: 316 - protein, 16 aa, Artificial Sequence
  Synthetic
AIRGGGGTYYADSVKG

SEQ ID NO: 317 - protein, 17 aa, Artificial Sequence
  Synthetic
VISGSGGNTYYADSVKG

SEQ ID NO: 318 - protein, 17 aa, Artificial Sequence
  Synthetic
RIIPTLGIANYAQKFQG

SEQ ID NO: 319 - protein, 17 aa, Artificial Sequence
  Synthetic
GISESGGGTYYADSVKG

SEQ ID NO: 320 - protein, 17 aa, Artificial Sequence
  Synthetic
RIIPISGRPNYAQKFQG

SEQ ID NO: 321 - protein, 19 aa, Artificial Sequence
  Synthetic
RIKSKIEGGTTDYAPPVKG

SEQ ID NO: 322 - protein, 17 aa, Artificial Sequence
  Synthetic
TISAGGSRTYYADSVKG

SEQ ID NO: 323 - protein, 17 aa, Artificial Sequence
  Synthetic
TIYRSGGRTYYADSVKG

SEQ ID NO: 324 - protein, 17 aa, Artificial Sequence
  Synthetic
TIYGSGVRTYYADGVKG

SEQ ID NO: 325 - protein, 17 aa, Artificial Sequence
  Synthetic
SISGSGISTYYADSVKG

SEQ ID NO: 326 - protein, 17 aa, Artificial Sequence
  Synthetic
AIYGAGGKTYYADSVKG

SEQ ID NO: 327 - protein, 17 aa, Artificial Sequence
  Synthetic
VIYGSGTRTYYADSVKG

SEQ ID NO: 328 - protein, 17 aa, Artificial Sequence
  Synthetic
VISSGGSYTYYADSVKG

SEQ ID NO: 329 - protein, 17 aa, Artificial Sequence
  Synthetic
TIYSSGSRTYYADSVKG

SEQ ID NO: 330 - protein, 17 aa, Artificial Sequence
  Synthetic
AIYGSGGDTYYADSVKG

SEQ ID NO: 331 - protein, 17 aa, Artificial Sequence
  Synthetic
VISYDGNIEYYANSVKG

SEQ ID NO: 332 - protein, 17 aa, Artificial Sequence
  Synthetic
AIKGSGGGTYYADSVKG

SEQ ID NO: 333 - protein, 17 aa, Artificial Sequence
  Synthetic
AISGSGTYTYYADSVKG

SEQ ID NO: 334 - protein, 17 aa, Artificial Sequence
  Synthetic
IISGTGGNTYYADSVKG

SEQ ID NO: 335 - protein, 17 aa, Artificial Sequence
  Synthetic
SISEGGGSTYYADSVKG

SEQ ID NO: 336 - protein, 18 aa, Artificial Sequence
  Synthetic
VISGGSGHTTYYADSVKG

SEQ ID NO: 337 - protein, 17 aa, Artificial Sequence
  Synthetic
SISGSGGKTYYADSVKG

SEQ ID NO: 338 - protein, 17 aa, Artificial Sequence
  Synthetic
TISGSGANTYYADSVKG

SEQ ID NO: 339 - protein, 17 aa, Artificial Sequence
  Synthetic
TIYAGGGRTYYADSVKG

SEQ ID NO: 340 - protein, 17 aa, Artificial Sequence
  Synthetic
SIYGSGHRTYYADSVKG

SEQ ID NO: 341 - protein, 9 aa, Artificial Sequence
  Synthetic
YDYNSYFDS

SEQ ID NO: 342 - protein, 12 aa, Artificial Sequence
  Synthetic
GLAGWEVYPFDV

SEQ ID NO: 343 - protein, 17 aa, Artificial Sequence
  Synthetic
SRYWYTKLISYTYGMDV

SEQ ID NO: 344 - protein, 9 aa, Artificial Sequence
  Synthetic
GDDQDAFDP

SEQ ID NO: 345 - protein, 18 aa, Artificial Sequence
  Synthetic
DRSVYLYYGYHYYEGFDV

SEQ ID NO: 346 - protein, 7 aa, Artificial Sequence
  Synthetic
GYRYFDH

SEQ ID NO: 347 - protein, 11 aa, Artificial Sequence
  Synthetic
VSVVWSYAFDY

SEQ ID NO: 348 - protein, 9 aa, Artificial Sequence
  Synthetic
VFPYGAVDV

SEQ ID NO: 349 - protein, 9 aa, Artificial Sequence
  Synthetic
GSEGSGFDV

SEQ ID NO: 350 - protein, 11 aa, Artificial Sequence
  Synthetic
TDYSLYYQFDY

SEQ ID NO: 351 - protein, 9 aa, Artificial Sequence
  Synthetic
GGDEDGFDV

SEQ ID NO: 352 - protein, 20 aa, Artificial Sequence
  Synthetic
EVADYWDGGVYYYYDGGFDV

SEQ ID NO: 353 - protein, 11 aa, Artificial Sequence
  Synthetic
VDHYSGYAFDL

SEQ ID NO: 354 - protein, 13 aa, Artificial Sequence
  Synthetic
AEVDDYWYLYMDV

SEQ ID NO: 355 - protein, 11 aa, Artificial Sequence
  Synthetic
TDHYSYYVMDY

SEQ ID NO: 356 - protein, 12 aa, Artificial Sequence
  Synthetic
TAYSYPYVYFDF

SEQ ID NO: 357 - protein, 17 aa, Artificial Sequence
  Synthetic
SDSYYGVYDGYYYGMDV

SEQ ID NO: 358 - protein, 10 aa, Artificial Sequence
  Synthetic
LKLYHAAFDI

SEQ ID NO: 359 - protein, 11 aa, Artificial Sequence
  Synthetic
VDHVHGYAFDY

SEQ ID NO: 360 - protein, 19 aa, Artificial Sequence
  Synthetic
VESYGYGEYITYNYYGFDV

SEQ ID NO: 361 - protein, 18 aa, Artificial Sequence
  Synthetic
DPAVTLDTYVYADHGFDV

SEQ ID NO: 362 - protein, 17 aa, Artificial Sequence
  Synthetic
EPPKYGAYSYYGYGFDP

SEQ ID NO: 363 - protein, 9 aa, Artificial Sequence
  Synthetic
YVRGVSFDV

SEQ ID NO: 364 - protein, 10 aa, Artificial Sequence
  Synthetic
YGWEYQAFDY

SEQ ID NO: 365 - protein, 10 aa, Artificial Sequence
  Synthetic
IAGLFDGFDV

SEQ ID NO: 366 - protein, 10 aa, Artificial Sequence
  Synthetic
SSRYGDTFDV

SEQ ID NO: 367 - protein, 15 aa, Artificial Sequence
  Synthetic
GLYAHHTYYYGGFDY

SEQ ID NO: 368 - protein, 18 aa, Artificial Sequence
  Synthetic
DYFWSPRGSIYDYDYFDV

SEQ ID NO: 369 - protein, 18 aa, Artificial Sequence
  Synthetic
DAGWSYYGLWIYYYYMDV

SEQ ID NO: 370 - protein, 15 aa, Artificial Sequence
  Synthetic
KLGGFDENYTYGMDV

SEQ ID NO: 371 - protein, 11 aa, Artificial Sequence
  Synthetic
VHHFGYYSLDV

SEQ ID NO: 372 - protein, 9 aa, Artificial Sequence
  Synthetic
HDPRYQLDV

SEQ ID NO: 373 - protein, 9 aa, Artificial Sequence
  Synthetic
GSYSDGFDV

SEQ ID NO: 374 - protein, 16 aa, Artificial Sequence
  Synthetic
HLGPPYLVTYSHGFDV

SEQ ID NO: 375 - protein, 10 aa, Artificial Sequence
  Synthetic
LKYGFYQFDY

SEQ ID NO: 376 - protein, 11 aa, Artificial Sequence
  Synthetic
WGGDGFYAMDY

SEQ ID NO: 377 - protein, 15 aa, Artificial Sequence
  Synthetic
IKKYGSAGYTVGFDS

SEQ ID NO: 378 - protein, 14 aa, Artificial Sequence
  Synthetic
IKVPWYYSSYYFDY

SEQ ID NO: 379 - protein, 8 aa, Artificial Sequence
  Synthetic
GRWGAFDY

SEQ ID NO: 380 - protein, 8 aa, Artificial Sequence
  Synthetic
YLFGHYMD

SEQ ID NO: 381 - protein, 9 aa, Artificial Sequence
  Synthetic
SRGWRGFDS

SEQ ID NO: 382 - protein, 18 aa, Artificial Sequence
  Synthetic
GGADVGYFYSTFYYYMDV

SEQ ID NO: 383 - protein, 12 aa, Artificial Sequence
  Synthetic
LVSYGAYYVFDY

SEQ ID NO: 384 - protein, 6 aa, Artificial Sequence
  Synthetic
DKRLTY

SEQ ID NO: 385 - protein, 9 aa, Artificial Sequence
  Synthetic
EWPGEYFDV

SEQ ID NO: 386 - protein, 9 aa, Artificial Sequence
  Synthetic
GAYGDDFDV

SEQ ID NO: 387 - protein, 18 aa, Artificial Sequence
  Synthetic
STPIPSYYPYIYSYYFDV

SEQ ID NO: 388 - protein, 12 aa, Artificial Sequence
  Synthetic
SVVTWVTYAFDY

SEQ ID NO: 389 - protein, 12 aa, Artificial Sequence
  Synthetic
SVRERYTYYMDY

SEQ ID NO: 390 - protein, 9 aa, Artificial Sequence
  Synthetic
GYPGEYFDY

SEQ ID NO: 391 - protein, 12 aa, Artificial Sequence
  Synthetic
GLVSFRGYAFDY

SEQ ID NO: 392 - protein, 12 aa, Artificial Sequence
  Synthetic
VRSYSGPGYFDY

SEQ ID NO: 393 - protein, 9 aa, Artificial Sequence
  Synthetic
ISPHYDFDV

SEQ ID NO: 394 - protein, 11 aa, Artificial Sequence
  Synthetic
EARVLGYSLDY

SEQ ID NO: 395 - protein, 10 aa, Artificial Sequence
  Synthetic
LRYGFYQFDY

SEQ ID NO: 396 - protein, 11 aa, Artificial Sequence
  Synthetic
LYPLHSYAFDH

SEQ ID NO: 397 - protein, 16 aa, Artificial Sequence
  Synthetic
VRFGYGSWRYRKYMDV

SEQ ID NO: 398 - protein, 12 aa, Artificial Sequence
  Synthetic
GPYSYYYYSFDY

SEQ ID NO: 399 - protein, 9 aa, Artificial Sequence
  Synthetic
VGWSWGFDP

SEQ ID NO: 400 - protein, 13 aa, Artificial Sequence
  Synthetic
VWHTDDYYEGFDV

SEQ ID NO: 401 - protein, 9 aa, Artificial Sequence
  Synthetic
VVGRAVFDI

SEQ ID NO: 402 - protein, 11 aa, Artificial Sequence
  Synthetic
GLSRKAFYFDY

SEQ ID NO: 403 - protein, 18 aa, Artificial Sequence
  Synthetic
DASWYGPFSYYYYYGFDV

SEQ ID NO: 404 - protein, 15 aa, Artificial Sequence
  Synthetic
HPSVGSASRSYYFDV

SEQ ID NO: 405 - protein, 17 aa, Artificial Sequence
  Synthetic
GEYGSSIAYSYYYGFDV

SEQ ID NO: 406 - protein, 11 aa, Artificial Sequence
  Synthetic
VGPLRLYGFDY

SEQ ID NO: 407 - protein, 17 aa, Artificial Sequence
  Synthetic
EVRHDSYYYYYYSGMDV

SEQ ID NO: 408 - protein, 16 aa, Artificial Sequence
  Synthetic
AAYFAGPAARYGYFDI

SEQ ID NO: 409 - protein, 10 aa, Artificial Sequence
  Synthetic
LVPWQLAFDV

SEQ ID NO: 410 - protein, 17 aa, Artificial Sequence
  Synthetic
EASYYVAGYYRYYGFDV

SEQ ID NO: 411 - protein, 15 aa, Artificial Sequence
  Synthetic
DPTWYVSGHYYGFDV

SEQ ID NO: 412 - protein, 12 aa, Artificial Sequence
  Synthetic
GWYPILYYAFDY

SEQ ID NO: 413 - protein, 13 aa, Artificial Sequence
  Synthetic
GAYPLGSYYGFDP

SEQ ID NO: 414 - protein, 12 aa, Artificial Sequence
  Synthetic
GWIPHSFYAFDY

SEQ ID NO: 415 - protein, 12 aa, Artificial Sequence
  Synthetic
GPGPGYWYAFDV

SEQ ID NO: 416 - protein, 11 aa, Artificial Sequence
  Synthetic
GLSRRAFYFDY

SEQ ID NO: 417 - protein, 12 aa, Artificial Sequence
  Synthetic
GLVSFKGYAFDY

SEQ ID NO: 418 - protein, 9 aa, Artificial Sequence
  Synthetic
ISHGWGFDV

SEQ ID NO: 419 - protein, 11 aa, Artificial Sequence
  Synthetic
VASSYHYAFDY

SEQ ID NO: 420 - protein, 11 aa, Artificial Sequence
  Synthetic
VEDTSYYGMDY

SEQ ID NO: 421 - protein, 10 aa, Artificial Sequence
  Synthetic
LEAGFYSLDI

SEQ ID NO: 422 - protein, 10 aa, Artificial Sequence
  Synthetic
VSGVVGALDI

SEQ ID NO: 423 - protein, 9 aa, Artificial Sequence
  Synthetic
TEYDGYFDV

SEQ ID NO: 424 - protein, 10 aa, Artificial Sequence
  Synthetic
VEDSHYSFDV

SEQ ID NO: 425 - protein, 17 aa, Artificial Sequence
  Synthetic
DRIGSSTYYGYYNGFDV

SEQ ID NO: 426 - protein, 8 aa, Artificial Sequence
  Synthetic
GGYLWLDP

SEQ ID NO: 427 - protein, 16 aa, Artificial Sequence
  Synthetic
VRISGYGPGYFGGFDV

SEQ ID NO: 428 - protein, 16 aa, Artificial Sequence
  Synthetic
IDTFAYYYSLYRAFDL

SEQ ID NO: 429 - protein, 15 aa, Artificial Sequence
  Synthetic
TKRYFAGGSYYWMDV

SEQ ID NO: 430 - protein, 10 aa, Artificial Sequence
  Synthetic
HGGGRFAMDV

SEQ ID NO: 431 - protein, 18 aa, Artificial Sequence
  Synthetic
DIYGITYDGYTYYDGMDV

SEQ ID NO: 432 - protein, 14 aa, Artificial Sequence
  Synthetic
HHDIAPSWIYYFDY

SEQ ID NO: 433 - protein, 12 aa, Artificial Sequence
  Synthetic
GLTRHYGYSFDH

SEQ ID NO: 434 - protein, 11 aa, Artificial Sequence
  Synthetic
VASSAEYAFDH

SEQ ID NO: 435 - protein, 14 aa, Artificial Sequence
  Synthetic
TGSSSNIGAGYDVH

SEQ ID NO: 436 - protein, 14 aa, Artificial Sequence
  Synthetic
TGSSSNIGAGFDVH

SEQ ID NO: 437 - protein, 14 aa, Artificial Sequence
  Synthetic
TGSSSNIGATYDVH

SEQ ID NO: 438 - protein, 11 aa, Artificial Sequence
  Synthetic
RASQSISNYLN

SEQ ID NO: 439 - protein, 11 aa, Artificial Sequence
  Synthetic
RASQSVYNNLA

SEQ ID NO: 440 - protein, 11 aa, Artificial Sequence
  Synthetic
RASQSVRNNLA

SEQ ID NO: 441 - protein, 11 aa, Artificial Sequence
  Synthetic
RASQDISTYLN

SEQ ID NO: 442 - protein, 12 aa, Artificial Sequence
  Synthetic
RASQSVSGSYLA

SEQ ID NO: 443 - protein, 11 aa, Artificial Sequence
  Synthetic
RASQRISNYLN

SEQ ID NO: 444 - protein, 11 aa, Artificial Sequence
  Synthetic
RASQSISRYLN

SEQ ID NO: 445 - protein, 11 aa, Artificial Sequence
  Synthetic
RASQSISSYLN

SEQ ID NO: 446 - protein, 12 aa, Artificial Sequence
  Synthetic
RASQSVSSNYLA

SEQ ID NO: 447 - protein, 11 aa, Artificial Sequence
  Synthetic
RASQDIGNYLN

SEQ ID NO: 448 - protein, 11 aa, Artificial Sequence
  Synthetic
RASQSISKYLN

SEQ ID NO: 449 - protein, 11 aa, Artificial Sequence
  Synthetic
RASQTITSYLN

SEQ ID NO: 450 - protein, 11 aa, Artificial Sequence
  Synthetic
RASQTISRYLN

SEQ ID NO: 451 - protein, 11 aa, Artificial Sequence
  Synthetic
RASQDISSYLN

SEQ ID NO: 452 - protein, 12 aa, Artificial Sequence
  Synthetic
RASQSVATGYLA

SEQ ID NO: 453 - protein, 12 aa, Artificial Sequence
  Synthetic
RASQSVGSNYLA

SEQ ID NO: 454 - protein, 12 aa, Artificial Sequence
  Synthetic
RASQSVSSSYLA

SEQ ID NO: 455 - protein, 12 aa, Artificial Sequence
  Synthetic
RASQSVRSSYLA

SEQ ID NO: 456 - protein, 11 aa, Artificial Sequence
  Synthetic
RASLSVGSNLA

SEQ ID NO: 457 - protein, 11 aa, Artificial Sequence
  Synthetic
RASQSVGSNLA

SEQ ID NO: 458 - protein, 12 aa, Artificial Sequence
  Synthetic
RASQSVRSNYLA

SEQ ID NO: 459 - protein, 11 aa, Artificial Sequence
  Synthetic
RASQTISNYLN

SEQ ID NO: 460 - protein, 11 aa, Artificial Sequence
  Synthetic
RASQSIGSYLN

SEQ ID NO: 461 - protein, 11 aa, Artificial Sequence
  Synthetic
RASQSLGTNLA

SEQ ID NO: 462 - protein, 11 aa, Artificial Sequence
  Synthetic
RASQSIRNYLN

SEQ ID NO: 463 - protein, 11 aa, Artificial Sequence
  Synthetic
RASQTISTYLN

SEQ ID NO: 464 - protein, 11 aa, Artificial Sequence
  Synthetic
RASQSVGSYLA

SEQ ID NO: 465 - protein, 14 aa, Artificial Sequence
  Synthetic
TGSSSNIGSGYDVH

SEQ ID NO: 466 - protein, 13 aa, Artificial Sequence
  Synthetic
TGSSSNIGAYVVH

SEQ ID NO: 467 - protein, 14 aa, Artificial Sequence
  Synthetic
TGSSSNIGAAFDVH

SEQ ID NO: 468 - protein, 13 aa, Artificial Sequence
  Synthetic
SGSSSNIGNNYVS

SEQ ID NO: 469 - protein, 12 aa, Artificial Sequence
  Synthetic
RASQSVASSYLA

SEQ ID NO: 470 - protein, 11 aa, Artificial Sequence
  Synthetic
RASQSVSNNLA

SEQ ID NO: 471 - protein, 11 aa, Artificial Sequence
  Synthetic
RASRSVSSNLA

SEQ ID NO: 472 - protein, 11 aa, Artificial Sequence
  Synthetic
RASQTISKYLN

SEQ ID NO: 473 - protein, 11 aa, Artificial Sequence
  Synthetic
RASQRIGKYLN

SEQ ID NO: 474 - protein, 11 aa, Artificial Sequence
  Synthetic
RASQSIATYLN

SEQ ID NO: 475 - protein, 11 aa, Artificial Sequence
  Synthetic
RASQSIYTYLN

SEQ ID NO: 476 - protein, 12 aa, Artificial Sequence
  Synthetic
RASQSVSSTYLA

SEQ ID NO: 477 - protein, 11 aa, Artificial Sequence
  Synthetic
RASQTIGNYLN

SEQ ID NO: 478 - protein, 11 aa, Artificial Sequence
  Synthetic
RASQSVYTNLA

SEQ ID NO: 479 - protein, 12 aa, Artificial Sequence
  Synthetic
RASQSVGSSYLA

SEQ ID NO: 480 - protein, 11 aa, Artificial Sequence
  Synthetic
RASRSLSNNLA

SEQ ID NO: 481 - protein, 11 aa, Artificial Sequence
  Synthetic
RASQSIANYLN

SEQ ID NO: 482 - protein, 11 aa, Artificial Sequence
  Synthetic
RASQSIGNYLN

SEQ ID NO: 483 - protein, 11 aa, Artificial Sequence
  Synthetic
RASQDISNYLN

SEQ ID NO: 484 - protein, 11 aa, Artificial Sequence
  Synthetic
RASQSISTYLN

SEQ ID NO: 485 - protein, 11 aa, Artificial Sequence
  Synthetic
RASQTIGIYLN

SEQ ID NO: 486 - protein, 11 aa, Artificial Sequence
  Synthetic
RASQTIGTYLN

SEQ ID NO: 487 - protein, 11 aa, Artificial Sequence
  Synthetic
RASQDIYNYLN

SEQ ID NO: 488 - protein, 11 aa, Artificial Sequence
  Synthetic
RASQSVSSNLA

SEQ ID NO: 489 - protein, 11 aa, Artificial Sequence
  Synthetic
RASQSISSNLA

SEQ ID NO: 490 - protein, 11 aa, Artificial Sequence
  Synthetic
RASQSVYSNLA

SEQ ID NO: 491 - protein, 11 aa, Artificial Sequence
  Synthetic
RASQSVGGNLA

SEQ ID NO: 492 - protein, 11 aa, Artificial Sequence
  Synthetic
RASQRISSYLN

SEQ ID NO: 493 - protein, 12 aa, Artificial Sequence
  Synthetic
RASQSVASPYLA

SEQ ID NO: 494 - protein, 11 aa, Artificial Sequence
  Synthetic
RASQSIYNYLN

SEQ ID NO: 495 - protein, 12 aa, Artificial Sequence
  Synthetic
RARQSVPSNYLA

SEQ ID NO: 496 - protein, 11 aa, Artificial Sequence
  Synthetic
RASQTISSYLN

SEQ ID NO: 497 - protein, 7 aa, Artificial Sequence
  Synthetic
GNNNRPS

SEQ ID NO: 498 - protein, 7 aa, Artificial Sequence
  Synthetic
DNTNRPS

SEQ ID NO: 499 - protein, 7 aa, Artificial Sequence
  Synthetic
VNNNRPS

SEQ ID NO: 500 - protein, 7 aa, Artificial Sequence
  Synthetic
AASSLQS

SEQ ID NO: 501 - protein, 7 aa, Artificial Sequence
  Synthetic
GASTRAT

SEQ ID NO: 502 - protein, 7 aa, Artificial Sequence
  Synthetic
GASSRAT

SEQ ID NO: 503 - protein, 7 aa, Artificial Sequence
  Synthetic
SASSLQS

SEQ ID NO: 504 - protein, 7 aa, Artificial Sequence
  Synthetic
TASSLQS

SEQ ID NO: 505 - protein, 7 aa, Artificial Sequence
  Synthetic
GASSLQS

SEQ ID NO: 506 - protein, 7 aa, Artificial Sequence
  Synthetic
AASILQS

SEQ ID NO: 507 - protein, 7 aa, Artificial Sequence
  Synthetic
GNKNRPS

SEQ ID NO: 508 - protein, 7 aa, Artificial Sequence
  Synthetic
GNRNRPS

SEQ ID NO: 509 - protein, 7 aa, Artificial Sequence
  Synthetic
GNTNRPS

SEQ ID NO: 510 - protein, 7 aa, Artificial Sequence
  Synthetic
DNYNRPS

SEQ ID NO: 511 - protein, 7 aa, Artificial Sequence
  Synthetic
ANNNRPS

SEQ ID NO: 512 - protein, 7 aa, Artificial Sequence
  Synthetic
DNNKRPS

SEQ ID NO: 513 - protein, 7 aa, Artificial Sequence
  Synthetic
AASSRAT

SEQ ID NO: 514 - protein, 7 aa, Artificial Sequence
  Synthetic
DASSRAT

SEQ ID NO: 515 - protein, 7 aa, Artificial Sequence
  Synthetic
VASSLQS

SEQ ID NO: 516 - protein, 7 aa, Artificial Sequence
  Synthetic
DASTRAT

SEQ ID NO: 517 - protein, 11 aa, Artificial Sequence
  Synthetic
QSYDRSLSGWV

SEQ ID NO: 518 - protein, 11 aa, Artificial Sequence
  Synthetic
QSYDYSLRVWV

SEQ ID NO: 519 - protein, 11 aa, Artificial Sequence
  Synthetic
QSYDSSLRAWV

SEQ ID NO: 520 - protein, 9 aa, Artificial Sequence
  Synthetic
QQSFSAPYT

SEQ ID NO: 521 - protein, 9 aa, Artificial Sequence
  Synthetic
QQYNDLPLT

SEQ ID NO: 522 - protein, 9 aa, Artificial Sequence
  Synthetic
QQYYTWPRT

SEQ ID NO: 523 - protein, 9 aa, Artificial Sequence
  Synthetic
QQSYSYPLT

SEQ ID NO: 524 - protein, 10 aa, Artificial Sequence
  Synthetic
QQYGGSLPYT

SEQ ID NO: 525 - protein, 9 aa, Artificial Sequence
  Synthetic
QQSYSPPYT

SEQ ID NO: 526 - protein, 9 aa, Artificial Sequence
  Synthetic
QQSYSYPVT

SEQ ID NO: 527 - protein, 9 aa, Artificial Sequence
  Synthetic
QQSYRPPYT

SEQ ID NO: 528 - protein, 10 aa, Artificial Sequence
  Synthetic
QQYGRSAPIT

SEQ ID NO: 529 - protein, 9 aa, Artificial Sequence
  Synthetic
QQSYTFPLT

SEQ ID NO: 530 - protein, 9 aa, Artificial Sequence
  Synthetic
QQSYSFPLT

SEQ ID NO: 531 - protein, 9 aa, Artificial Sequence
  Synthetic
QQSYTYPLT

SEQ ID NO: 532 - protein, 9 aa, Artificial Sequence
  Synthetic
QQTYSTPWT

SEQ ID NO: 533 - protein, 9 aa, Artificial Sequence
  Synthetic
QQSYSFPPT

SEQ ID NO: 534 - protein, 10 aa, Artificial Sequence
  Synthetic
QQYESSPPFT

SEQ ID NO: 535 - protein, 9 aa, Artificial Sequence
  Synthetic
QQYNDWPLT

SEQ ID NO: 536 - protein, 9 aa, Artificial Sequence
  Synthetic
QQYGSSPYT

SEQ ID NO: 537 - protein, 10 aa, Artificial Sequence
  Synthetic
QQYETSPPVT

SEQ ID NO: 538 - protein, 9 aa, Artificial Sequence
  Synthetic
QQSYSTPWT

SEQ ID NO: 539 - protein, 9 aa, Artificial Sequence
  Synthetic
QQYASLPLT

SEQ ID NO: 540 - protein, 9 aa, Artificial Sequence
  Synthetic
QQYNNLPLT

SEQ ID NO: 541 - protein, 9 aa, Artificial Sequence
  Synthetic
QQYHNWPPT

SEQ ID NO: 542 - protein, 9 aa, Artificial Sequence
  Synthetic
QQYFSAPYT

SEQ ID NO: 543 - protein, 10 aa, Artificial Sequence
  Synthetic
QQYGSSPAVT

SEQ ID NO: 544 - protein, 9 aa, Artificial Sequence
  Synthetic
QQYGASPQT

SEQ ID NO: 545 - protein, 10 aa, Artificial Sequence
  Synthetic
QQYGSSPEYT

SEQ ID NO: 546 - protein, 9 aa, Artificial Sequence
  Synthetic
QQSYSTPYT

SEQ ID NO: 547 - protein, 9 aa, Artificial Sequence
  Synthetic
QQYYEAPPT

SEQ ID NO: 548 - protein, 9 aa, Artificial Sequence
  Synthetic
QQSYTVPLT

SEQ ID NO: 549 - protein, 9 aa, Artificial Sequence
  Synthetic
QQYYNAPIT

SEQ ID NO: 550 - protein, 11 aa, Artificial Sequence
  Synthetic
QSYDTSLSAWV

SEQ ID NO: 551 - protein, 11 aa, Artificial Sequence
  Synthetic
QSYDSSLKTWV

SEQ ID NO: 552 - protein, 11 aa, Artificial Sequence
  Synthetic
QSYDGRLGVSV

SEQ ID NO: 553 - protein, 11 aa, Artificial Sequence
  Synthetic
QSYDRTLSSYV

SEQ ID NO: 554 - protein, 11 aa, Artificial Sequence
  Synthetic
QSYDSSLSVWV

SEQ ID NO: 555 - protein, 11 aa, Artificial Sequence
  Synthetic
QSYDYSLSGWV

SEQ ID NO: 556 - protein, 11 aa, Artificial Sequence
  Synthetic
QSYDTRLSAWV

SEQ ID NO: 557 - protein, 11 aa, Artificial Sequence
  Synthetic
GTWDSSLSARV

SEQ ID NO: 558 - protein, 9 aa, Artificial Sequence
  Synthetic
QQYGSSQLT

SEQ ID NO: 559 - protein, 9 aa, Artificial Sequence
  Synthetic
QQSYSAPYT

SEQ ID NO: 560 - protein, 9 aa, Artificial Sequence
  Synthetic
QQYGNYPPT

SEQ ID NO: 561 - protein, 11 aa, Artificial Sequence
  Synthetic
QSYDSGLSGWV

SEQ ID NO: 562 - protein, 11 aa, Artificial Sequence
  Synthetic
QSYDVSLGVWV

SEQ ID NO: 563 - protein, 10 aa, Artificial Sequence
  Synthetic
QHYGGSPGYT

SEQ ID NO: 564 - protein, 9 aa, Artificial Sequence
  Synthetic
QQSFTFPLT

SEQ ID NO: 565 - protein, 9 aa, Artificial Sequence
  Synthetic
QQSYTIPPT

SEQ ID NO: 566 - protein, 9 aa, Artificial Sequence
  Synthetic
QQYYNWPLT

SEQ ID NO: 567 - protein, 9 aa, Artificial Sequence
  Synthetic
QQSYSYPIT

SEQ ID NO: 568 - protein, 9 aa, Artificial Sequence
  Synthetic
QQSYSVPLT

SEQ ID NO: 569 - protein, 9 aa, Artificial Sequence
  Synthetic
QQSYSTPLT

SEQ ID NO: 570 - protein, 9 aa, Artificial Sequence
  Synthetic
QQSYRYPLT

SEQ ID NO: 571 - protein, 9 aa, Artificial Sequence
  Synthetic
QQYGTTPQT

SEQ ID NO: 572 - protein, 9 aa, Artificial Sequence
  Synthetic
QQYAALPIT

SEQ ID NO: 573 - protein, 9 aa, Artificial Sequence
  Synthetic
QQSYRYPYT

SEQ ID NO: 574 - protein, 9 aa, Artificial Sequence
  Synthetic
QQYYSAPLT

SEQ ID NO: 575 - protein, 9 aa, Artificial Sequence
  Synthetic
QQYNDWPYT

SEQ ID NO: 576 - protein, 9 aa, Artificial Sequence
  Synthetic
QQYSDYPFT

SEQ ID NO: 577 - protein, 9 aa, Artificial Sequence
  Synthetic
QQYAYAPWT

SEQ ID NO: 578 - protein, 9 aa, Artificial Sequence
  Synthetic
QQSYAPPYT

SEQ ID NO: 579 - protein, 9 aa, Artificial Sequence
  Synthetic
QQYYYWPIT

SEQ ID NO: 580 - protein, 9 aa, Artificial Sequence
  Synthetic
QQYNAWPYT

SEQ ID NO: 581 - protein, 9 aa, Artificial Sequence
  Synthetic
QQYFSTPPT

SEQ ID NO: 582 - protein, 9 aa, Artificial Sequence
  Synthetic
QQTYTIPLT

SEQ ID NO: 583 - protein, 9 aa, Artificial Sequence
  Synthetic
QQSYTYPVT

SEQ ID NO: 584 - protein, 9 aa, Artificial Sequence
  Synthetic
QQSFTAPVT

SEQ ID NO: 585 - protein, 9 aa, Artificial Sequence
  Synthetic
QQSYTYPIT

SEQ ID NO: 586 - protein, 9 aa, Artificial Sequence
  Synthetic
QQYNNWPYT

SEQ ID NO: 587 - protein, 9 aa, Artificial Sequence
  Synthetic
QQSDGFPPT

SEQ ID NO: 588 - protein, 9 aa, Artificial Sequence
  Synthetic
QQSYTVPWT

SEQ ID NO: 589 - protein, 9 aa, Artificial Sequence
  Synthetic
QQYNDYPLT

SEQ ID NO: 590 - protein, 9 aa, Artificial Sequence
  Synthetic
QQYSNWPLT

SEQ ID NO: 591 - protein, 9 aa, Artificial Sequence
  Synthetic
QQYYESPIT

SEQ ID NO: 592 - protein, 9 aa, Artificial Sequence
  Synthetic
QQSDSPPWT

SEQ ID NO: 593 - protein, 9 aa, Artificial Sequence
  Synthetic
QQYYSSPLT

SEQ ID NO: 594 - protein, 10 aa, Artificial Sequence
  Synthetic
QQYSSSPTYT